SEQUENCE LISTING (418 sequences)

SEQ ID NO: 1 - RNA, 675 nt, synthetic construct
  source 1..675: mol_type = other RNA
ttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 2 - RNA, 612 nt, synthetic construct
  source 1..612: mol_type = other RNA
tttcccctgttcgtaactaagtgtgtgcccaatctcctcactcctgctggcttcaccgaccggcagtgtccaaaatgctaggtgaatcccctccctttcctctgggcttctgcccagcttcctccccccagcctgacgtgacacaggctgtgcaaagaccccgcgaaagctgccaaaagtggcaattgtgggtcccccctttgtaaaggcgtcgagtctttctccctcaaggctagacccgtcagtgaattctgtcgggcaactagtgacgccactgcacgcctctgacctcggccgcggagtgctgccccccaagtcgtgcccctgaccacaagttgtgctgtctggcaaacattgtctgtgagaatgttccgctgtggctgccaagcctggcaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggttcgccagggatctgagcgtaggctaattgtctaagggtattttcatttcccattctttctttcttgttcata

SEQ ID NO: 3 - RNA, 678 nt, synthetic construct
  source 1..678: mol_type = other RNA
tgaaccgttacgcaccactcagttggtgtttggtggcaccaatgatggaacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaagg

SEQ ID NO: 4 - RNA, 601 nt, synthetic construct
  source 1..601: mol_type = other RNA
tttaagtgttgtgcccaatctcttgactcctgctggaaccaccgaccagtagtgtccaaaatgccaggtggaaaatcctcccttcccctctgggcttcatgcccggcatcctccccccagcctgacgtgccacaggctgtgcaaagaccccgcgaaagctgccaaaagtggcaattgtgggtcccccctttgtcaaggcgtcgagtctttctcccttaaggctagtcctgtcagtgaactctgtcgggcaactagtgacgccactgcatgcctccgacctcggccgcggagtgctgccccccaagtcatgcccctgaccacaagttgtgctgtctggcaaacattgtctgtgagaatgttccgctgtggctgccaagcctggtaacaggctgccccagtgtgcgtaattctcatccagacttcggtctggcaacttgctgttaagacatggcgtaaggggcgtgtgccaacgccctggaacgagtgtccactctaataccccgaggaatgctacgcaggtacccctggctcgccagggatctgagcgtaggctaattgtctaagggtattttcatttcccaccctcttcttcttgttcata

SEQ ID NO: 5 - RNA, 675 nt, synthetic construct
  source 1..675: mol_type = other RNA
tttgctcagcgtaacttctccgggttacgtggagaccaaaaggctacggagactcgggctacggccctggagcacctaggtgctcctaaagacgttagaagttgtacaaactcgcccaatagggccccccaaccaggggggtagcgggcaagcacttctgtttccccggtatgatctcataggctgtacccacggctgaaagagagattatcgttacccgcctcactacttcgagaagcccagtaatggttcatgaagttgatctcgttgacccggtgtttcccccacaccagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttgttccttttatcaagttttatggtgacaactcctggtagacgttttattgcgtttattgagagatttccaacaattgaacagactagaaccacttgttttatcaaaccctcacagaataagataaca

SEQ ID NO: 6 - RNA, 545 nt, synthetic construct
  source 1..545: mol_type = other RNA
cccccttttccctaaccccaaccgccgtatctggtggcggcaagacacacgggtctttccctctaaagcacaaatgtgtgtgtgtcccaggtcctcctgcgctcggtgcgggagtgctcccacccaactgttgtaagcctgtccaacgtgtcgtcctggcaagactatgacgtcgcatgttccgctgcggatgccgaccgggtaaccggttccccagtgtgtgtagtgcgatcttccaggttcccctggttggcgttgtccagaaactgcttcaggtaagtggggtgtgcccaatccctgcaaaggttgattctctcaccaccttaggaatgctccggaggtaccccagcaacagctgggatctgaccggaggctaattgtctacgggtggtgtttcctttttctttttccacaacttcattgctgacaactcactgactacccatttgctctcttgtgcctttctgctctggttcaagctccttgattgtttttgattgctttctattgctttcttccactcaacccttgctcagttcaaagtc

SEQ ID NO: 7 - RNA, 605 nt, synthetic construct
  source 1..605: mol_type = other RNA
ttcaagtgtgtgtgctcgtaatcttgactcctgccggaatgccgcccggttcagtgaacaaacagctaggcaagtccctcccttcccctgtggtcggttctcaccggccaccatccctcccccagcctgacgtgttacaggctgtgcaaagcccccgcgaaagctgctcacgtggcaattgtgggtcccccctttgtcaagacaccgagtctttctcccttaaggctagcccggtcccacgaacgtggaactggcaactagtggtgtcactacacgcctccgacctcggacgcggagtgctgttccccaagctgtaaccctgacccaagactgtgctgcctggcaagcaccgtctgggaagatgttccgctgtggctgccaaacctggtaacaggtgccccagtgtgtgtagtcttcctccagtctccggactggcagtcttgtgtaaagatgcagtgtaaggttcaagtgccaaatccctggaaggagtgaccctctactgccctaggaatgctgtgcaggtacccccaacttcggttggggatctgagcacaggctaattgtctacgggtagtttcatttcccatcctctcttttttggcatc

SEQ ID NO: 8 - RNA, 665 nt, synthetic construct
  source 1..665: mol_type = other RNA
gcccgtccccctcaccctcttttccggtggccacgcccgggccaccgatacttcccttcactccctcgggactgttggggaggaacacaacagggctcccctgtatttcctcttcccattccccctttcctaaccccaaccgccgtatctggtggcggtaagacacacgggtctttccctctaaagcacaattgtgtgtgtgtcccaggtcctcctgcgttcggtgcgggagtgctcccacccaactgttgtaagcctgtccaacgtgtcgtcctggcaagactatgacgtcgcatgttccgctgtggatgccgaccgggtaaccggttccccagtgtgtgtagtgcgatcttccaggttctcctggttggcgttgtccagaaactgcttcgggtaagtggggtgtgcccaatccctacaagggttgattctttcaccaccttaggaatgctccggaggtaccccagcaacagctgggatctgaccggaggctaattgtctacgggtggtgtttccattttctttttcacacaacttcattgctgacaactcactgactaatcacttgctctcttgtgcctttctgctctggttcaagttccttgattgtttgtttgattgcttttcactgctttcttcccacaatccttgctcagttcaaagtc

SEQ ID NO: 9 - RNA, 546 nt, synthetic construct
  source 1..546: mol_type = other RNA
ttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 10 - RNA, 619 nt, synthetic construct
  source 1..619: mol_type = other RNA
gtaacttcaagtgtgtgtgctcgtaatcttgactcctgccggaatgccgcccggttcagtgaacaaacagctaggcaagtccctcccttcccctgtggtcggttctcaccggccaccatccctcccccagcctgacgtgttacaggctgtgcaaagcccccgcgaaagctgctcacgtggcaattgtgggtcccccctttgtcaagacaccgagtctttctcccttaaggctagcccggtcccacgaacgtggaactggcaactagtggtgtcactacacgcctccgacctcggacgcggagtgctgttccccaagctgtaaccctgacccaagactgtgctgcctggcaagcaccgtctgggaagatgttccgctgtggctgccaaacctggtaacaggtgccccagtgtgtgtagtcttcctccagtctccggactggcagtcttgtgtaaagatgcagtgtaaggttcaagtgccaaatccctggaaggagtgaccctctactgccctaggaatgctgtgcaggtacccccaacttcggttggggatctgagcacaggctaattgtctacgggtagtttcatttcccatcctctcttttttggcatcatggcgaac

SEQ ID NO: 11 - RNA, 781 nt, synthetic construct
  source 1..781: mol_type = other RNA
ccccctcaccctcttttccggcggcgcatgttcgcgtcgtcgtaagtctggattcccaaggcccactcggttcaacttcggtttccggacaaatacaaagaacctcagtcctcttggtactttctcgcctgagtctacaaagcgagaaacctgcccctctaacgccagacgagcggcataaaactcgaacttctggcacgttccaccaccccttctcctatcccaacccccattgcgctctcaaggtcgcgcttttccgagactagctcggattcaaaaagttcctggcaccctttaccccttcaggcccttaaggtaggaactgaccttgtgctgtgatctcggtgcggaagtgctactgcgtagtgattgtaagatccttttgtggttctgccctggcaaggctacagagtgctgtgatccgctgcggatgccatcctggtaacaggacccccagtgtgcgcaacagtatgttcacggtcttccgtgttcaccacattcggaacactgctttcgtgaaacagtgtgtgtccaatccctgtgatcagtatcaaccacaccacctaggaatgctaggaaggtaccccggttcgccgggatctgatcctaggctaattgtctacggtggtgctcctttttattttccacttcaactcactggttacaactgcttgatttctgtgtttgctgcttttctctgctctcactgccattctcaagtgttcacactgtccaagctcctttggttgttcgcttccacttgccactgtcaactcttgtc

SEQ ID NO: 12 - RNA, 741 nt, synthetic construct
  source 1..741: mol_type = other RNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 13 - RNA, 21 nt, synthetic construct
  source 1..21: mol_type = other RNA
tgaggagatcgtcgaggttgg

SEQ ID NO: 14 - RNA, 21 nt, synthetic construct
  source 1..21: mol_type = other RNA
tcatcctcgtctccctcccag

SEQ ID NO: 15 - RNA, 22 nt, synthetic construct
  source 1..22: mol_type = other RNA
gtagataaaatattggtacctg

SEQ ID NO: 16 - RNA, 21 nt, synthetic construct
  source 1..21: mol_type = other RNA
cggggcagctcagtacaggat

SEQ ID NO: 17 - RNA, 21 nt, synthetic construct
  source 1..21: mol_type = other RNA
tagataaaatattggtacctg

SEQ ID NO: 18 - RNA, 22 nt, synthetic construct
  source 1..22: mol_type = other RNA
agagttaactcaaaatggacta

SEQ ID NO: 19 - RNA, 21 nt, synthetic construct
  source 1..21: mol_type = other RNA
gcccggagagctgggagccag

SEQ ID NO: 20 - RNA, 22 nt, synthetic construct
  source 1..22: mol_type = other RNA
tgtaaacatcctcgactggaag

SEQ ID NO: 21 - RNA, 21 nt, synthetic construct
  source 1..21: mol_type = other RNA
aaggtttggatagatgcaata

SEQ ID NO: 22 - RNA, 21 nt, synthetic construct
  source 1..21: mol_type = other RNA
ctagactgaagctccttgagg

SEQ ID NO: 23 - RNA, 21 nt, synthetic construct
  source 1..21: mol_type = other RNA
cataaagtagaaagcactact

SEQ ID NO: 24 - RNA, 22 nt, synthetic construct
  source 1..22: mol_type = other RNA
acaggtgaggttcttgggagcc

SEQ ID NO: 25 - RNA, 21 nt, synthetic construct
  source 1..21: mol_type = other RNA
aggcaagatgctggcatagct

SEQ ID NO: 26 - RNA, 22 nt, synthetic construct
  source 1..22: mol_type = other RNA
tggagtgtgacaatggtgtttg

SEQ ID NO: 27 - RNA, 21 nt, synthetic construct
  source 1..21: mol_type = other RNA
ccaacctcgacgatctcctca

SEQ ID NO: 28 - RNA, 21 nt, synthetic construct
  source 1..21: mol_type = other RNA
ctgggagggagacgaggatga

SEQ ID NO: 29 - RNA, 22 nt, synthetic construct
  source 1..22: mol_type = other RNA
caggtaccaatattttatctac

SEQ ID NO: 30 - RNA, 21 nt, synthetic construct
  source 1..21: mol_type = other RNA
atcctgtactgagctgccccg

SEQ ID NO: 31 - RNA, 21 nt, synthetic construct
  source 1..21: mol_type = other RNA
caggtaccaatattttatcta

SEQ ID NO: 32 - RNA, 22 nt, synthetic construct
  source 1..22: mol_type = other RNA
tagtccattttgagttaactct

SEQ ID NO: 33 - RNA, 21 nt, synthetic construct
  source 1..21: mol_type = other RNA
ctggctcccagctctccgggc

SEQ ID NO: 34 - RNA, 22 nt, synthetic construct
  source 1..22: mol_type = other RNA
cttccagtcgaggatgtttaca

SEQ ID NO: 35 - RNA, 21 nt, synthetic construct
  source 1..21: mol_type = other RNA
tattgcatctatccaaacctt

SEQ ID NO: 36 - RNA, 21 nt, synthetic construct
  source 1..21: mol_type = other RNA
cctcaaggagcttcagtctag

SEQ ID NO: 37 - RNA, 21 nt, synthetic construct
  source 1..21: mol_type = other RNA
agtagtgctttctactttatg

SEQ ID NO: 38 - RNA, 22 nt, synthetic construct
  source 1..22: mol_type = other RNA
ggctcccaagaacctcacctgt

SEQ ID NO: 39 - RNA, 21 nt, synthetic construct
  source 1..21: mol_type = other RNA
agctatgccagcatcttgcct

SEQ ID NO: 40 - RNA, 22 nt, synthetic construct
  source 1..22: mol_type = other RNA
caaacaccattgtcacactcca

SEQ ID NO: 41 - RNA, 40 nt, synthetic construct
  source 1..40: mol_type = other RNA
acaacaacaaacaacaaaacaacaaacaacacaaaaccaa

SEQ ID NO: 42 - RNA, 37 nt, synthetic construct
  source 1..37: mol_type = other RNA
aaaaacaaaaaacaaaaaccacacaaatggtcgccga

SEQ ID NO: 43 - RNA, 108 nt, synthetic construct
  source 1..108: mol_type = other RNA
aacttttagttattttcggcgaccatttgtgtggtgagctctaagtgttgatcagcgggcgcagctgcatatgacctctgcgtgtgcctggtaattgactaagatatc

SEQ ID NO: 44 - RNA, 58 nt, synthetic construct
  source 1..58: mol_type = other RNA
taatagaaaaacaaaaaacaaaaaccacacaaatggtcgccgaaactactgaaagcat

SEQ ID NO: 45 - RNA, 61 nt, synthetic construct
  source 1..61: mol_type = other RNA
tgataatagaaaaacaaaaaacaaaaaccacacaaatggtcgccgaaactactgaaagcat

SEQ ID NO: 46 - RNA, 88 nt, synthetic construct
  source 1..88: mol_type = other RNA
taatagaaaaacacaaaccaaacaccattgtcacactccaaaaaaaacaaacaaccacacaaatggtcgccgaaactactgaaagcat

SEQ ID NO: 47 - RNA, 183 nt, synthetic construct
  source 1..183: mol_type = other RNA
taatagaaaaacacaaaccaaacaccattgtcacactccaactcatgatcagtacagaacaaacaccattgtcacactccaagctgacatacatatcataactctactatagacaaacaccattgtcacactccaaaaaaaacaaacaaccacacaaatggtcgccgaaactactgaaagcat

SEQ ID NO: 48 - protein, 7 aa, synthetic construct
GVSLPDY

SEQ ID NO: 49 - protein, 5 aa, synthetic construct
WGSET

SEQ ID NO: 50 - protein, 12 aa, synthetic construct
HYYYGGSYAMDY

SEQ ID NO: 51 - protein, 11 aa, synthetic construct
RASQDISKYLN

SEQ ID NO: 52 - protein, 7 aa, synthetic construct
HTSRLHS

SEQ ID NO: 53 - protein, 9 aa, synthetic construct
QQGNTLPYT

SEQ ID NO: 54 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 55 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 56 - protein, 15 aa, synthetic construct
GGGGSGGGGSGGGGS

SEQ ID NO: 57 - protein, 18 aa, synthetic construct
GSTSGSGKPGSGEGSTKG

SEQ ID NO: 58 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 485 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 503 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 8 aa, synthetic construct
GRTFGTYT

SEQ ID NO: 62 - protein, 8 aa, synthetic construct
ISWSAGRT

SEQ ID NO: 63 - protein, 17 aa, synthetic construct
DPWTSDSDYDRLTMYDY

SEQ ID NO: 64 - protein, 8 aa, synthetic construct
GFTFSMYS

SEQ ID NO: 65 - protein, 8 aa, synthetic construct
ISTGARDT

SEQ ID NO: 66 - protein, 12 aa, synthetic construct
GDLRYGPDGYDY

SEQ ID NO: 67 - protein, 8 aa, synthetic construct
GSDFLVDA

SEQ ID NO: 68 - protein, 7 aa, synthetic construct
MDIGGVT

SEQ ID NO: 69 - skipped

SEQ ID NO: 70 - protein, 8 aa, synthetic construct
GSDASTNN

SEQ ID NO: 71 - protein, 7 aa, synthetic construct
IRTGGST

SEQ ID NO: 72 - protein, 11 aa, synthetic construct
WTGSPGALSDY

SEQ ID NO: 73 - protein, 8 aa, synthetic construct
GRTFINYA

SEQ ID NO: 74 - protein, 8 aa, synthetic construct
ISRSGGRT

SEQ ID NO: 75 - protein, 15 aa, synthetic construct
ATVWEFTDGADQYDY

SEQ ID NO: 76 - protein, 8 aa, synthetic construct
GGTFSTYG

SEQ ID NO: 77 - protein, 8 aa, synthetic construct
ITGSGVGT

SEQ ID NO: 78 - protein, 16 aa, synthetic construct
GHRPGWAVIRADAYEY

SEQ ID NO: 79 - protein, 8 aa, synthetic construct
GFSFSRVG

SEQ ID NO: 80 - protein, 8 aa, synthetic construct
ISSDGSRT

SEQ ID NO: 81 - protein, 4 aa, synthetic construct
PGNS

SEQ ID NO: 82 - protein, 8 aa, synthetic construct
GFPFSSSF

SEQ ID NO: 83 - protein, 7 aa, synthetic construct
IYSDGST

SEQ ID NO: 84 - protein, 5 aa, synthetic construct
VTGSI

SEQ ID NO: 85 - protein, 6 aa, synthetic construct
GRTTYN

SEQ ID NO: 86 - protein, 8 aa, synthetic construct
IDLYGRAT

SEQ ID NO: 87 - protein, 18 aa, synthetic construct
DTSLPLGVLTESQRLYGA

SEQ ID NO: 88 - protein, 126 aa, synthetic construct
QVQLVESGGGLVQAGGSLRLSCAASGRTFGTYTMGWFRQAPGREREFVAAISWSAGRTYYADSMKGRFTISRDNAKNTVYLQMNSLKPEDTAVYYCAADPWTSDSDYDRLTMYDYWGQGTQVTVSS

SEQ ID NO: 89 - protein, 121 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSS

SEQ ID NO: 90 - protein, 111 aa, synthetic construct
QLQLVESGGGLVQPGGSLRLSCAASGSDFLVDATTWFRQAPGNQREFVAIMDIGGVTEYADSVKGRFTISRDHTKNTVYLQMNSLKVEDTAVYYCNTRGLWGQGTLVTVSS

SEQ ID NO: 91 - protein, 119 aa, synthetic construct
QVQLVESGGGLVQPGGSLRLSCAASGSDASTNNMAWYRQAPGKEREFVALIRTGGSTHVADSMKGRFTISRENAKNTVYLQMNSLKPEDTAVYYCVIWTGSPGALSDYWGQGTQVTVSS

SEQ ID NO: 92 - protein, 124 aa, synthetic construct
QVQLVESGGGLVQPGGSLRLSCAASGRTFINYAAGWFRQAPGKEREFVARISRSGGRTDYADSVKGRFTISRDNAKSTVYLQMNSLRPEDTAVYYCAEATVWEFTDGADQYDYWGQGTQVTVSS

SEQ ID NO: 93 - protein, 125 aa, synthetic construct
QVQLVESGGGLVQPGGSLRLSCVASGGTFSTYGMGWFRQAPGKEREFVAVITGSGVGTQYADSVKDRFTISRENAKNTVYLQMNTLKLEDTAVYYCVSGHRPGWAVIRADAYEYWGQGTQVTVSS

SEQ ID NO: 94 - protein, 113 aa, synthetic construct
QLQLVESGGGLVQPGESLRLSCAASGFSFSRVGMNWYRQAPGKERELVASISSDGSRTNYAHFVKGRFTISRDNVKNMVYLQMNSLKLEDTAVYYCNVPGNSWGQGTQVTVSS

SEQ ID NO: 95 - protein, 113 aa, synthetic construct
EVQLVESGGGLVQPGGSLRLSCAASGFPFSSSFMSWVRQAPGKGLEWVSTIYSDGSTYYADSVKGRFTISRDNAKKTAYLQMNSLKAEDTAVYYCATVTGSIRGQGTQVTVSS

SEQ ID NO: 96 - protein, 125 aa, synthetic construct
QVQLVESGGGLVQAGASLRLSCAASGRTTYNMGWFRHAPGKDREFVAAIDLYGRATRYANSVKGRFTISRDNAKNTVYLQMNSLKPEDTAVYYCAADTSLPLGVLTESQRLYGAWGQGTQVTVSS

SEQ ID NO: 97 - protein, 124 aa, synthetic construct
EVQLVESGGGLVQAGGSLRLSCAASGFTFDDYAIGWFRQAPGKGREGVLCIRIFDRHTYSADSVKGRFTISSDNAQNTVYLHMNSLKPEDTAVYYCAAGSFWGCTRPEGDMDYWGKGTQVTVSS

SEQ ID NO: 98 - protein, 125 aa, synthetic construct
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 99 - protein, 125 aa, synthetic construct
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 100 - protein, 127 aa, synthetic construct
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 101 - protein, 262 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSS

SEQ ID NO: 102 - protein, 274 aa, synthetic construct
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 103 - protein, 270 aa, synthetic construct
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAGGGGSGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 104 - protein, 268 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 105 - protein, 268 aa, synthetic construct
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSS

SEQ ID NO: 106 - protein, 266 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 107 - protein, 266 aa, synthetic construct
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSS

SEQ ID NO: 108 - protein, 272 aa, synthetic construct
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 109 - protein, 272 aa, synthetic construct
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 110 - protein, 403 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSS

SEQ ID NO: 111 - protein, 413 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 112 - protein, 413 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 113 - protein, 413 aa, synthetic construct
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSA

SEQ ID NO: 114 - protein, 413 aa, synthetic construct
MDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSEVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSS

SEQ ID NO: 115 - protein, 413 aa, synthetic construct
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSIT

SEQ ID NO: 116 - protein, 413 aa, synthetic construct
EVQLVESGGGLVQPGGSLRLSCAASGFTFDDYAIGWFRQAPGKGREGVACIRIFDRHTYYADSVKGRFTISSDNSKNTVYLQMNSLRAEDTATYYCAAGSFWGCTRPEGDMDYFGQGTLVQVQSAGGGGSGGGGSGGGGSGGGGSMDVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGGSGGGGSGGGGSGGGGSQVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSS

SEQ ID NO: 117 - protein, 4 aa, synthetic construct
LPET

SEQ ID NO: 118 - protein, 6 aa, synthetic construct
GQQQLG

SEQ ID NO: 119 - protein, 4 aa, synthetic construct
GGGC

SEQ ID NO: 120 - protein, 8 aa, synthetic construct
GGGSCAAA

SEQ ID NO: 121 - protein, 10 aa, synthetic construct
GGGSGQQQLG

SEQ ID NO: 122 - protein, 129 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGSCAAA

SEQ ID NO: 123 - protein, 134 aa, synthetic construct
QVQLVESGGGLVQPGGSLRLSCAASGFTFSMYSMSWVRQAPGKRLEWVSSISTGARDTAYADSVKGRFTISRDNADNTLYLHMNNLKPEDTAVYYCANGDLRYGPDGYDYRGQGTQVTVSSGGGGSGGGGSCAS

SEQ ID NO: 124 - protein, 121 aa, synthetic construct
QVQLVESGGGLVQPGGSLRLSCAASGFTFSMYSMSWVRQAPGKRLEWVSSISTGARDTAYADSVKGRFTISRDNADNTLYLHMNCLKPEDTAVYYCANGDLRYGPDGYDYRGQGTQVTVSS

SEQ ID NO: 125 - RNA, 1458 nt, synthetic construct
  source 1..1458: mol_type = other RNA
atggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaaga

SEQ ID NO: 126 - RNA, 1458 nt, synthetic construct
  source 1..1458: mol_type = other RNA
atggcactacccgttacagcattactattacctctcgccctcctcctccacgccgccagaccagatatccagatgacacagaccacctctagcctcagcgccagcctcggcgatagagtgacaatctcttgcagagcctcccaggacatcagcaagtacctcaattggtaccagcagaagcccgacggcaccgtgaagctcctgatctaccacaccagcaggctgcacagcggcgtgccatctagattctctggatctggatctggcaccgattacagcctgaccatcagcaatctggagcaggaggacatcgccacctacttctgccagcaggggaataccctgccctacacctttggcggcggcacaaagctggagattacaggtggtggtggttctgggggaggagggtctggtggtggtggtagtgaagtgaaactgcaggaatctggacctggcctggtggccccttctcagtcgctgagcgtgacatgcaccgtgtctggcgtgagcctgccggattacggcgtgagctggattcggcagcctcctcggaagggcctggaatggctgggcgtgatctggggctccgagaccacctactacaacagcgccctgaagtccaggctgaccatcatcaaggacaacagcaagtcccaggtgttcctgaagatgaacagcctgcagaccgatgacacggccatctactactgcgccaagcactactactacggaggctcctacgccatggattactggggccagggaacctccgtgacagtgtcttctacaacaacaccagctcctaggcctcctacacctgctcctacaattgcttctcagcctctgtctctgagaccagaagcttgtagaccagctgctggaggagctgtgcatacaagaggcctggatttcgcctgtgacatttacatctgggctccactggccggcacctgtggcgtgctgctgctgagcctggtgatcaccctgtactgcaagaggggcagaaagaagctgctgtacatcttcaagcagcctttcatgcgccccgtgcagacaacacaggaggaagatggctgcagctgtagatttcctgaagaagaggagggcggctgcgagctgagagtgaaattttctagaagcgccgacgcccctgcctaccagcagggccagaatcagttatataacgaactgaatctgggccggcgggaggagtacgacgtgctggataagagaagaggcagagatcctgaaatgggaggaaaacctagaagaaagaatccgcaggagggcttatataatgaactgcagaaggacaagatggcggaggcctacagcgagatcggcatgaagggcgagagaagaagaggcaagggccacgatggcctgtaccagggcctgagcacggccaccaaggatacctacgatgccctgcacatgcaggccctgcctcctaga

SEQ ID NO: 127 - RNA, 1458 nt, synthetic construct
  source 1..1458: mol_type = other RNA
atggcattaccagttacagccctccttcttcctctcgccctcttgctccacgccgccaggccagatatccagatgacacagaccaccagcagcctcagcgccagcctcggcgatagagtgacaatctcttgcagagcctcccaggacatcagcaagtacctcaattggtaccagcagaagcccgacggcaccgtgaagttgctgatctaccacaccagcagactgcacagcggagtgccttctagattctctggaagcggctccggcacagactactcgctgaccatcagcaacctggagcaggaggacatcgccacctacttctgccagcagggcaataccctgccctacaccttcggcggcggcacaaaactggagattacaggtggtggtggtagtggtggtgggggaagtggtggtggtggttctgaagtgaaactgcaggaatcagggcctggcctggtggccccttctcagtcgctgagcgtgacctgtaccgtgtcaggcgtgagcctgccagattacggagtgagctggattagacagccccctcggaagggcctggaatggctgggcgtgatctggggcagcgagaccacctactacaacagcgccctgaagtccaggctgaccatcatcaaggacaacagcaagtcccaggtgttcctgaagatgaacagcctgcagaccgacgacaccgccatctactactgcgccaagcactactactacggcggcagctacgccatggattactggggccagggcacctctgtgacggtgtcttctacaacaacacctgctcctaggcctcctacaccagctcctacaattgcttctcagccgctgagtctgcggccagaagcttgtaggcctgctgcaggaggagctgtgcatacaagaggactggactttgcctgtgatatctacatctgggcccctctggccggcacctgcggcgtgctgctgctgagcctggtgatcaccctgtactgcaagcgcggccggaagaagctgctgtacatcttcaagcagcccttcatgaggcccgtgcagacaacacaggaggaggatggctgctcctgtagatttcctgaagaggaggagggcggctgcgagctgagagtgaaattttctagaagcgccgatgcccctgcctaccagcagggccagaaccagttatataacgaactgaacctgggcagaagagaggagtacgacgtgctggataagagaagaggcagagatccagaaatgggaggaaagcctagaagaaagaatccgcaggagggcctatataatgaactgcagaaggacaagatggccgaggcctacagcgagatcggcatgaagggcgagagaagaagaggcaagggccacgatggactgtatcaggggctgagcaccgccaccaaggatacctacgatgccctgcacatgcaggcgctgcctcctaga

SEQ ID NO: 128 - RNA, 1458 nt, synthetic construct
  source 1..1458: mol_type = other RNA
atggcattaccagttacagccctcctcctccctctcgccctccttctccacgccgccagaccagatatccagatgacccagaccacctctagcctcagcgccagcctcggcgatagagtgacaatctcttgcagagcctcccaggacatcagcaagtacctcaattggtaccagcagaagcccgacggcaccgtgaagctcctgatctaccacaccagcaggctgcactctggcgtgccatctagattttctggatctggcagcggcaccgactacagcctgaccatcagcaacctggagcaggaggacatcgccacctacttctgccagcaggggaataccctgccctacacctttggcggcggcacaaagctggagattacaggtggtggtggttctggtggtggtggtagtggtggtgggggatctgaagtgaaactgcaggaatctggacctggcctggtggccccttctcagtctctgagcgtgacatgcaccgtgtccggcgtgagcctgccggattacggcgtgagctggattcggcagcctcctagaaagggcctggaatggctgggcgtgatctggggcagcgagaccacctactacaacagcgcgctgaagtccaggctgaccatcatcaaggacaacagcaagtcccaggtgttcctgaagatgaacagcctgcagaccgatgacaccgccatctactactgtgcgaagcactactactacggcggctcctacgccatggattactggggccagggcaccagcgtgacggtgtcttctacaacaacacctgctcctagacctcctacaccagctcctacaattgcttctcagcctctgtcgctgagacctgaagcatgtagacctgctgctggaggagctgtgcatacaagaggactggattttgcctgcgacatctacatctgggccccgctggccggcacctgcggcgtgctgctgctgagcctggtgatcaccctgtactgcaagaggggcaggaagaagctgctgtacatcttcaagcagcccttcatgaggcctgtgcagaccacccaggaggaggatggctgcagctgcagatttcctgaagaagaggagggaggctgcgagctgagagtgaaattttccaggagcgccgacgctcctgcctaccagcagggccagaatcagttatataatgaactgaacctgggccggcgggaggagtacgacgtgctggataagagaagaggcagagatcctgaaatgggaggaaagcctagaagaaagaatccgcaggagggcctatataacgaactgcagaaggacaagatggccgaggcctattccgagatcggaatgaagggcgagagaagaagaggcaagggccacgatggcctgtaccagggcctgagcacggccaccaaggatacctacgacgccctgcacatgcaggccctgcctcctaga

SEQ ID NO: 129 - RNA, 1458 nt, synthetic construct
  source 1..1458: mol_type = other RNA
atggccctacccgttacagctctactgctgccgctggcactcttgctgcacgcagccagacctgacattcagatgacgcagactacgtcgtccctgtcagcgtcccttggggatcgggttactataagttgccgcgccagccaggatataagtaaatacctcaattggtatcagcaaaagccggatggtaccgtgaagctcctcatctaccatacgtcccggctccactctggtgtgccgagcaggttctccgggagtgggagtgggacagactacagcctcacgatcagcaatctagagcaggaggacatcgctacttacttttgccagcaggggaatacccttccctacactttcggtggcggcacgaagttggagataactggaggcggcgggtctggcggcggtgggtcaggcggtgggggctcggaggtgaagctgcaagaaagcggtccagggctggtggccccttcacagtcccttagtgtgacctgcactgtcagcggagtctcactgcccgattacggggtgagctggatcaggcaaccgccgaggaaggggctggaatggttaggtgtcatctgggggtcagagactacttactataacagcgctttgaagtctagactgactatcattaaggataactccaaatcccaagtcttcctcaagatgaacagtttacagacagatgacacagccatctactactgtgccaagcactactactacggaggctcatatgcgatggactattggggccaaggtacgtcggttacagtaagctcgacgaccactcccgcacctcggcctcccacccctgctcctaccatcgccagtcaacccctgtccctgcggcctgaggcctgtcgtcccgcggccgggggtgcggtccatacgcgcggacttgactttgcgtgtgatatctacatttgggcccccttagctggcacatgtggtgttctcctgctctccctggtcatcaccttgtactgcaagcgtggtcgcaagaagttgctatatatatttaagcagcccttcatgcggccagtccagactactcaggaagaagatggctgtagttgccggttccccgaggaagaggaaggcggctgcgagctgcgggttaagttctctcgcagtgccgatgcccctgcgtatcagcaggggcagaatcaactgtataacgagttgaatcttggccgtcgggaagagtacgatgttctcgataaacggcggggtcgagatcccgagatgggagggaagccccggagaaagaacccacaagaggggctttataacgagctgcagaaagacaagatggccgaggcctactcggagatcgggatgaagggagaacggcgccggggtaagggccacgacggcctttaccaagggttaagtaccgcgacaaaagatacttatgacgcactgcacatgcaggcgttgccgccaagg

SEQ ID NO: 130 - RNA, 1458 nt, synthetic construct
  source 1..1458: mol_type = other RNA
atggccttgccggtcactgcattgctgctgcctctcgcactgctgcttcatgccgcaaggcctgacattcagatgactcagacaaccagctcgctcagcgcatcgctgggggatcgcgtgacgatcagttgccgtgcatcccaggatatttccaagtacctcaactggtaccagcagaagccagatggtacagtaaaattgctgatataccatacatccagattacactcaggcgtaccctcgcgctttagtggctccggaagtggcaccgattactctttgaccatcagtaatctcgagcaagaagacattgcaacgtacttctgccaacaggggaacactctcccatacacctttgggggaggtactaagctcgagattacgggtggcggggggtcgggcggtggcggctctggaggcggagggagcgaggttaaactgcaggagtcaggccctgggcttgtagctcccagtcagtcactgtctgtgacttgtactgtgtcaggtgtttccttgccagactatggggtatcctggatccgccagccaccccgtaaaggtctggagtggctcggggtcatatggggctccgaaactacttattacaactcggcattgaagagcaggctgacaattattaaagataatagtaagtcgcaggtgttccttaaaatgaactccctgcaaactgatgatacggccatatactattgtgccaaacactattattacggcggcagctatgctatggactattggggtcagggcacatctgttaccgttagttcgacgacaacgcccgcaccgcgccctccaaccccagctccgacgattgcatcgcagcccctctcactgcgccccgaggcctgtcggccagccgcaggtggcgctgtgcatacacgggggctggacttcgcatgcgatatttatatctgggctccactggccgggacgtgcggagttcttctcctgtcactggtgatcaccctgtattgtaagcgcgggcggaagaagctgctatatatttttaagcagccctttatgaggcctgtgcagacgacacaggaagaagacgggtgcagttgccgattccctgaggaagaggaggggggctgcgagctgcgcgtgaaattttcgcgctccgctgacgcacctgcgtaccagcaaggccagaatcaactctacaatgagttgaatttggggagacgcgaagagtacgacgtcctcgacaagcgacgagggagggatcctgaaatgggaggaaagcctcgcagaaagaaccctcaggaggggttgtacaatgagttacagaaggacaagatggcggaagcctactccgagatagggatgaagggcgagcggaggcgggggaagggccacgacgggctttatcaaggtctgtccacagccaccaaggatacctatgacgctctgcatatgcaggccctgcccccacgc

SEQ ID NO: 131 - RNA, 1458 nt, synthetic construct
  source 1..1458: mol_type = other RNA
atggcccttccagtgacagcattgttgcttcctttggcactgctgttacatgccgcccgccccgacattcagatgacacagacaacttcctcactgagtgccagtctcggagacagagtgaccatcagctgtcgggcctcccaagatatcagtaaataccttaactggtaccagcagaagcccgatgggacagttaaacttctcatctatcataccagcaggctgcacagcggcgttcctagcagatttagtggtagcgggagcggcactgattacagcctgacaattagcaatctcgagcaggaggatatcgctacctacttttgccagcagggtaacaccctgccctacactttcggggggggaactaaactggagataacagggggtggcgggagcgggggaggtggcagtggggggggcgggagtgaagtgaaactccaggagagcgggccaggtctcgtggccccctctcaatctttgtcagtgacttgcacagtctctggcgtttccctgcccgactatggcgtctcatggattcgacagccccctcgaaaggggttagagtggctcggagtgatatggggctccgaaaccacctattacaattccgctttaaagagcagattgacaatcatcaaggataattccaagtctcaggtgtttttgaaaatgaactcccttcagactgatgacactgcaatctactactgtgccaaacattactattacggggggagttacgccatggactactgggggcagggcacaagtgtgactgtctcctccaccaccactcctgctccccgacctcctacacctgctccaactatcgcctctcagccactgagcctgcgacccgaggcttgcaggcctgcagctggaggcgctgtccataccagaggtctggacttcgcctgcgatatctacatctgggccccactagccggtacctgcggcgtcctcttattgagcctcgtgatcactctttactgcaagagaggccgcaagaagctgttgtacatattcaaacagccttttatgcggcctgtgcagactacacaggaggaagacgggtgctcatgtagattccctgaggaagaggagggtggatgcgagcttcgggtgaagttcagccgaagcgctgacgcccccgcataccaacagggccagaaccaactctacaatgagttgaatttgggccgcagggaagaatacgacgtgctggataaacgaaggggccgagaccccgaaatggggggcaagcctcggcggaagaatccccaggaaggcttgtacaatgagttgcagaaagataaaatggccgaggcctactccgagattggtatgaagggcgaaagaaggcggggaaaagggcacgacgggttgtatcaggggctgagtaccgcaacaaaggacacctatgatgccctacatatgcaggctctacctcccaga

SEQ ID NO: 132 - RNA, 1458 nt, synthetic construct
  source 1..1458: mol_type = other RNA
atggctcttcccgtgacagccttgctgctcccattggctctcctactgcacgcagcacgacccgatattcagatgacccagactacctcttccctcagcgcctccctcggggaccgagtgactatctcctgtcgggcatcccaggatattagtaagtacctcaattggtaccagcaaaagcccgacgggacagtcaaactattaatataccacacctctagactacatagtggtgttccttctcggttctccgggagcggctctgggactgactacagtctcaccataagcaacctggagcaggaggacatagccacatacttctgtcagcagggcaacaccctgccctataccttcggagggggcaccaaactagagattactggcggagggggttcaggaggaggcgggagcgggggcggtggaagtgaggtgaagttacaggagtccgggcctgggctcgtcgccccttcccagagcctgtcagttacctgtactgtgagcggagtctcattacccgactacggagtcagctggatccgccagccccccaggaagggactggagtggctgggcgtgatctggggttctgagaccacatattataattctgctctaaaatctaggctgacaatcatcaaagacaacagcaagagccaggtgtttctgaagatgaactcattacaaacagatgacactgctatctactactgcgcaaagcactactattatggtgggagctatgccatggattattgggggcaggggaccagcgtcactgtctccagtacaacaacccccgctccacgacctccaaccccagcacccactattgcttcacagcccctgagtttacggcccgaggcatgtcgccctgccgctggaggggctgttcataccagaggactggacttcgcctgtgatatctacatttgggccccactggccggaacatgtggcgttctcctgctctccctcgtcataacactctattgcaagagaggcaggaagaaacttctctatatctttaaacagccttttatgaggcctgtgcagactacacaggaagaggatggatgttcctgccgattcccagaggaggaggagggcgggtgtgagctacgagtgaagttctcacgctctgcagatgcccccgcttaccagcaggggcagaatcagctctacaacgagctgaatctcggacgccgagaagaatacgacgtgctggacaaacgcaggggtagagaccccgagatggggggaaaacctaggcgcaaaaacccccaggagggcctgtataacgagctgcaaaaggataagatggccgaagcttacagtgagatcggcatgaagggggagcgccgccgcggtaaagggcacgacggtctgtaccagggacttagcacagctaccaaggacacctacgatgctctccacatgcaggcattaccacccagg

SEQ ID NO: 133 - RNA, 1458 nt, synthetic construct
  source 1..1458: mol_type = other RNA
atggccttgcccgtcactgccctattacttcctctcgccctgttgctccacgccgctcgccccgacatccaaatgacacagaccacctcttccctatccgcatccttgggagatagggttaccatctcctgtagggcatcccaggatataagcaaatatttgaattggtatcagcaaaaacctgatggtacagtgaagctactcatttatcacacatcccggttgcattccggcgtgccctcccgcttcagcggaagcggttccgggactgactattcacttactataagtaatctagagcaagaggacattgcaacttatttctgccagcaaggaaacaccctaccctacacatttggaggaggaacaaaattggaaatcaccgggggcgggggttcaggtggtggtggaagcggtggtggtggatcagaggttaagttgcaagagagcggccccggccttgtggctcccagtcagagtctatcagtgacctgtaccgtgtctggcgttagtctcccagactatggagttagctggatcagacaaccaccacgcaagggactggagtggcttggagttatatggggatctgagaccacatattacaattctgcccttaagtcccgactaacaatcattaaagacaactctaagagccaggttttcctcaaaatgaactctttgcagaccgatgacaccgctatttactattgtgccaagcactactattacggtgggtcatacgctatggattactgggggcagggcacatcagtcactgtgagctctacaaccacacccgctcccaggcctccaacccccgcccccactatcgcctctcaacctctcagcttaaggccagaggcttgtcgacctgctgccggcggagctgttcatacccgcggtttggacttcgcttgcgacatatacatttgggcaccactcgccggcacctgcggtgtgctcctcctttctctcgtgattaccctgtattgcaagcgaggcagaaaaaaattattatatatttttaaacagcccttcatgcggcctgtgcagactacacaagaagaagatgggtgcagctgcaggttccctgaggaggaggaaggcggctgtgaacttcgggtcaaattttcccgcagtgccgatgctcctgcataccaacaaggtcaaaaccagctgtacaatgagttaaatcttggacggagagaagagtatgatgtcttggacaagcggcgaggtcgggatcctgagatgggcggaaagccacggcgcaagaacccacaagaaggtctctacaacgagcttcagaaagataaaatggctgaagcttatagcgaaattgggatgaaaggagaaagacgcaggggaaagggacacgacggactttatcaaggtctatctaccgctaccaaagatacttatgacgccctccacatgcaggcacttcctccacgc

SEQ ID NO: 134 - RNA, 1458 nt, synthetic construct
  source 1..1458: mol_type = other RNA
atggccttgccagtaaccgcacttctgctgccactagcactgctgctgcatgcagcccgaccagatatacaaatgacccagacaacctcgagtttgagtgcctcccttggagatcgcgtaactatcagctgtcgggctagtcaggacatttcaaagtacctcaactggtatcagcagaaacctgatggaactgtgaagctgctgatttaccatacatcacgcttacactccggagtgccctcacgtttctccggatccggttccggaactgattatagtcttaccattagcaaccttgagcaggaagacatagccacctatttctgccaacaaggaaatactttgccttatacctttgggggcggcactaagctggaaatcacaggtggcgggggaagcgggggcgggggaagtggcggtggggggtctgaagtgaagctccaagaatctggaccaggtttagttgcaccgagccaaagcctatccgtgacctgcactgtttccggggtgtctcttccagattacggagtgtcctggatacgccagccgccgcgaaaagggctcgagtggttgggcgtcatatggggcagcgaaacgacctattataacagtgccctgaaaagccggctaacgatcattaaagataactctaagtcacaagtattcctcaagatgaactcccttcagacggatgatacggccatatactactgtgctaagcattattattatggcgggtcatacgcgatggactactggggccaggggacatccgtcaccgtatcctctacgacgacaccggcaccaagacctcctactcctgcacccaccatagccagccagcctttgtcactgagacccgaggcttgtagaccagctgccggtggcgcagtgcacacccgaggcttggactttgcctgtgacatttacatctgggcaccacttgcagggacttgcggggtactcttattgagcctcgtaatcacactgtactgcaagagaggacgcaaaaaattgctatatatttttaaacagccttttatgcgccctgttcagactacgcaggaggaagatggatgttcttgtaggtttcccgaagaagaggagggcgggtgtgagctaagagtaaaattcagtagatctgcggatgcaccggcctaccagcagggtcagaaccagttatacaacgagctaaacttaggacgcagagaagagtacgatgtattagataaacgccgtggtcgcgatcctgaaatgggtggtaagcctcgtcggaagaatcctcaggagggactgtacaacgaactacagaaagataagatggctgaggcttactccgagattggaatgaagggagagcgcagaaggggaaagggacatgacggcctgtaccaaggactgagcactgcaactaaggatacctacgacgctctgcacatgcaggccctgcccccaaga

SEQ ID NO: 135 - RNA, 1458 nt, synthetic construct
  source 1..1458: mol_type = other RNA
atggctctaccagtcacagcactactcctccctcttgccctccttcttcacgccgccaggccagatatccagatgacacagaccacctcctccctttccgcctctctcggggaccgagtcactatttcttgtcgggcttctcaggatatcagtaagtaccttaattggtaccagcagaagcccgacgggacagtgaagctcctgatctaccacaccagtaggctccactctggtgtcccctccagatttagtgggtcagggagtgggaccgactactccctgaccatctccaatctggagcaggaggatatcgccacctacttctgtcagcaggggaataccctgccatatacatttggagggggcaccaagctggagattactggtggtggtggatcaggaggaggtggaagtggaggaggtggttctgaagtgaaacttcaggaatctggacctggcctggtggctcccagccagtccctgagtgtgacttgcaccgtgtctggcgtgtctctgccagactacggtgtgagctggatccgccagccacccaggaaaggcctggagtggctgggcgtgatctggggcagtgagaccacctactataatagtgctttgaagtccaggctgaccatcatcaaggacaactccaagagtcaggtgttcttgaagatgaacagcctgcagactgatgataccgcaatctactattgcgcaaagcactattattatggggggtcttacgccatggattactgggggcaggggactagtgttactgtgagtagcactactacccctgcccctagacctccaacaccagctcctaccattgcaagtcagcctctgtctctgagaccagaggcttgtagacctgcagctggtggagctgtgcatactcggggactggattttgcatgtgacatttacatctgggctcctctggccggcacctgcggcgtcctcctcctgagtctggtgatcactctgtactgcaagcgcggaaggaagaagctgctgtacatcttcaagcagcccttcatgcgcccagtgcagaccactcaggaggaggacggctgcagctgccggtttccagaggaggaagaaggagggtgtgagctgagggtgaagttttcacgctcagctgacgcccctgcttatcagcagggccagaatcagttatataatgaactgaatctgggcaggagggaggagtacgatgtgctggataaacggagaggcagggatcccgaaatgggaggtaagcctcggcggaagaatcctcaggagggtctatataacgaactccagaaggataagatggccgaggcttactccgagatcgggatgaaaggggagaggcgccggggtaaaggccacgatggcctttaccagggcctctccaccgcaacaaaggatacctatgatgccctgcatatgcaggccctgcctccccgg

SEQ ID NO: 136 - RNA, 1458 nt, synthetic construct
  source 1..1458: mol_type = other RNA
atggcactacccgttaccgctctcttgctccctttggccctcctcctccacgccgccagacccgatatccagatgactcagactacatctagtttgagtgcatctctcggagatcgggtcaccatttcttgtcgggcttctcaggatatcagtaagtaccttaattggtaccagcagaagcccgacgggacagtgaagctcctgatctaccacaccagtaggctccactctggtgtcccctccagatttagtgggtcagggagtgggaccgactactccctgaccatctccaatctggagcaggaggatatcgccacctacttctgccagcaggggaataccctgccatatacatttggagggggcaccaagctggagattactggtggtggtggatcaggaggtggtggtagtggaggaggagggtcagaggtgaaactgcaggagagcggtcctggtctcgttgccccaagtcagtccctgtccgtgacatgtaccgtgtctggcgtgtctctgccagactacggagtgtcatggatcagacagccacccaggaaaggcctggagtggctgggagtgatttggggcagcgagaccacatattataactccgccctgaagtccaggctgaccatcatcaaggacaactccaagagtcaggtgttcttgaagatgaatagcctgcagacagatgacacggccatctactactgtgcgaagcactactactatggcggcagctacgccatggattactgggggcaggggacatctgtgactgtgagttcaacaaccactccagctccacggcctcctactccagctccaaccattgcaagtcagcctctgagtctgagaccagaggcttgtagacctgcagctggtggagctgttcatactcgcgggctggatttcgcctgcgatatctacatctgggctcctctggccggcacctgcggcgtcctcctcctgagcctggtgatcactctgtactgcaagcgcggaaggaagaagctgctgtacatcttcaagcagcccttcatgcgcccagtgcagaccactcaggaggaggacggctgcagctgccggtttccagaggaggaggaaggaggctgtgagctgagagtgaagttttctcgcagcgcagatgcccctgcctaccagcagggccagaatcagttatataatgaactgaatctgggccgcagggaggagtatgatgtgctggataagcggaggggcagggatcccgaaatggggggtaagcctcggcggaagaatcctcaggagggtctatataacgaactccagaaggataagatggccgaggcttactccgagatcgggatgaaaggggagaggcgccggggtaaaggccacgatggcctttaccagggcctctccaccgcaacaaaggatacctatgatgccctgcatatgcaggccctgcccccccgg

SEQ ID NO: 137 - RNA, 1458 nt, synthetic construct
  source 1..1458: mol_type = other RNA
atggccctaccagtgacagccctcctcctccctttggccttgttgctccacgcagcaaggccagatattcagatgacacagacaaccagcagcctctccgcctctcttggagacagggtgaccatttcttgtcgggcttctcaggatatcagtaagtaccttaattggtaccagcagaagcccgacgggacagtgaagttgctgatctaccacactagcaggctgcatagcggtgtcccttccagattctcgggatctggaagcggcaccgactacagcctgactatcagcaacctggagcaggaggacatcgccacatatttctgccagcaggggaataccctgccctatacatttggcgggggcacaaagctggagattacaggtggcggaggctctggaggaggaggatcaggcgggggcggatctgaagtgaaacttcaggaatccggccccggcctggtcgctccttcccagagcctgtccgtcacctgtacagtctccggcgtgtccctgccagattatggggtgagttggattcgccagccccctagaaaggggctggaatggttaggcgtgatttgggggtcagagaccacctactacaactctgccctcaagtcacgcctgaccattatcaaggacaatagtaagagccaggtgtttctgaagatgaatagtcttcagaccgatgacaccgctatttactattgtgccaagcactactactatggcggcagctacgccatggattactgggggcaggggactagtgttactgtgagtagcactactacccctgcccctagacctccaactccagctccaaccattgcaagtcagcctctgagtctgagaccagaggcttgtagacctgcagctggtggagctgttcatacaaggggcctggattttgcctgtgacatttacatctgggcccctctggcaggaacatgtggcgtgctcctcctgtctctggtgatcaccctgtactgtaagagaggcaggaagaagctgctgtacatcttcaagcagccattcatgagacctgtgcagacaacacaggaagaggatggctgctcctgcaggttccctgaggaggaggaggggggctgtgaactgcgcgtcaaattctcccggtctgccgatgcccctgcttatcagcaggggcagaatcagttatataatgaactgaatctcggcagacgggaggagtatgacgtgctggataagcggaggggcagggatcccgaaatggggggtaagcctcggcggaagaatcctcaggagggtctatataacgaactccagaaggataagatggccgaggcttactccgagatcgggatgaaaggggagaggcgccggggtaaaggccacgatggcctttaccagggcctctccaccgcaacaaaggatacctatgatgccctgcatatgcaggccctgcccccccgg

SEQ ID NO: 138 - RNA, 1458 nt, synthetic construct
  source 1..1458: mol_type = other RNA
atggcattaccagttaccgccctcctcctccccctcgccctcctcctccacgccgcccggcccgacatccagatgacccagaccaccagcagcctcagcgccagcctcggcgaccgggtgaccatctcttgccgggccagccaggacatcagcaagtacctcaattggtaccagcagaagccggacggcaccgtgaagctcctgatctaccacaccagccggctgcactccggggtgcccagccggttcagcggcagcggcagcggcaccgactactccctgaccatcagcaacctggagcaggaggacatcgccacctacttctgccagcaggggaacaccctgccctacaccttcggcggcggcaccaagctggagatcaccggcggcggcggcagcggcggcggcggcagcggcgggggcggcagcgaggtgaagctgcaggagagcggccccggcctggtggcccccagccagagcctgtccgtgacctgcaccgtgtccggcgtgagcctgcccgactacggcgtgtcctggatccgccagccccccaggaagggcctggagtggctgggcgtgatctggggcagcgagaccacctactacaacagcgcgctgaagtccaggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgtccagcaccaccacccccgccccccggccccccacccccgcccccaccatcgccagccagcccctgagcctgcggccggaggcctgcaggcccgccgccggcggcgccgtgcacacccggggcctggacttcgcctgcgacatctacatctgggcccccctggccggcacctgcggcgtgctgctgctgagcctggtgatcaccctgtactgcaagcggggccggaagaagctgctgtacatcttcaagcagcccttcatgcgccccgtgcagaccacccaggaggaggacggctgcagctgccgcttccccgaggaggaggagggcggctgcgagctgcgggtgaagttctcccgcagcgccgacgcccctgcgtaccagcagggccagaaccagctgtacaacgagctgaacctgggccggcgcgaggagtacgacgtgctggacaagcgccggggccgggaccccgagatgggcggcaagccccggcgcaagaacccccaggagggcctgtacaacgagctgcagaaggacaagatggccgaggcctacagcgagatcggcatgaagggcgagcggcggcggggcaagggccacgacggcctgtaccagggcctgtccaccgccaccaaggacacctacgacgccctgcacatgcaggccctgcccccccgg

SEQ ID NO: 139 - RNA, 1458 nt, synthetic construct
  source 1..1458: mol_type = other RNA
atggccctgcccgtgaccgccctgctgctccccctggccctgctgctgcacgcagcaaggccagacatccagatgacccagactacgagcagcctgagcgcctccctgggcgacagggtgacaatcagctgccgggccagtcaggacatcagcaagtacctgaactggtaccagcagaagcctgacggcaccgtgaagctgctgatttatcacacctccaggctgcacagcggcgtgccctccagattctccggcagcggctccggcaccgactacagcctgaccatctccaacctggagcaggaggatatcgccacctacttctgccagcaggggaacaccctgccctatacctttgggggcggaaccaagctggagatcaccggcggtggcggatccggcggcggcgggagcggcggcggaggatctgaggtcaagctgcaggagtctggccctgggctggtggccccaagccagagcctgtccgtgacatgtaccgtgtccggcgtgtctctgcctgactacggcgtgtcatggatcaggcagcctcctcgcaaggggctggagtggctgggcgtgatctggggcagcgagaccacctactacaacagtgccctgaagtcccggctgaccatcatcaaggacaacagcaagagccaggtgttcttgaagatgaacagcctccagaccgacgacaccgccatttactactgtgccaagcactattactatggcgggtcctacgccatggattactggggccagggcaccagtgtgaccgtgtccagcaccacgactcctgccccacgcccacccactccagcccctaccatcgccagccagccactgagcctgcggcctgaggcctgcaggccagcggctggcggggcggtgcacacccgcgggctggacttcgcctgcgacatctacatctgggcccccctcgccggcacctgcggcgtcctcctcctgagcctggtgatcactctgtactgcaagaggggccgcaagaagctgctgtatatcttcaagcagcctttcatgcggcccgtgcagaccactcaggaggaggacggctgcagctgccggtttcctgaggaggaggaggggggctgtgagctgcgggtgaagttcagccgctccgccgacgcccccgcctaccagcagggccagaatcagctgtacaacgagctgaatctgggccgcagggaggagtacgacgtgctcgacaagcggcgggggcgggaccccgagatgggcggcaagcccaggcggaaaaacccccaggaggggctgtacaatgagctccagaaggacaaaatggccgaggcttacagcgagatcggcatgaagggggagaggcggaggggcaaggggcatgacggcctgtaccaggggctgtccacagccaccaaggacacctacgacgccctgcacatgcaggccctgcctcctagg

SEQ ID NO: 140 - RNA, 1458 nt, synthetic construct
  source 1..1458: mol_type = other RNA
atggccctgcccgtgaccgccctgctgcttcccctggccctgctgctgcacgccgcaaggccagacatccagatgacccagactaccagttctctgtccgcctctctgggcgacagagtgacgatcagttgccgggccagtcaggacatcagcaagtacctgaactggtaccagcagaagcctgacggcaccgtgaagctgctgatctaccacaccagccggctgcacagcggagtgcccagccggttcagcggctccgggtccggcactgattacagcctgaccatctcaaacctggagcaggaggacatcgccacttacttctgccagcagggcaataccctgccctacaccttcggaggcggcaccaagctggagatcaccggcggcggcggctccggcggcggtggctccggcgggggcggctccgaggtgaagctgcaggagagtggcccaggcctggttgcccccagccagagcctgagcgtgacatgcaccgtgtcaggcgtgagtcttcctgactacggggtgagctggatcaggcagccccctaggaagggcctggagtggctgggggtgatctggggctcagagaccacatactacaactctgccctcaagagcaggctgaccatcatcaaggacaactccaagtcccaggtgttcttgaagatgaacagcctgcagaccgacgataccgccatttactactgtgccaagcactactactatggcgggtcctacgccatggactattgggggcaggggactagtgtgactgtgagctccaccaccaccccagcccccaggccccctactcctgcccctaccatcgccagccagcccctgagcctgcggcctgaggcctgcaggccagccgccgggggcgccgtgcacaccaggggcctggactttgcctgtgacatttacatctgggcccctctggccggcacctgcggcgtgctgctgctgagcctggtgatcaccctgtactgcaagaggggcaggaagaagctgctgtatatcttcaagcagcccttcatgcggccagtgcagaccactcaggaggaggacggctgcagctgccgcttccctgaggaggaggagggcggctgcgagctgcgggtgaagttctcccgcagcgccgatgcccctgcctaccagcagggccagaatcagctgtacaatgagctgaatctgggccgtcgggaggagtacgacgtgctggataagcgcaggggcagagatcctgagatgggcggcaagccccgcaggaagaatcctcaggagggtctgtacaatgagctgcagaaggacaagatggctgaggcttattcagagatcggcatgaagggcgagaggcggcggggcaagggccacgatggcctctatcaggggctgagcacggccaccaaggatacatacgacgccctgcacatgcaggcgctgcctccccgg

SEQ ID NO: 141 - RNA, 1458 nt, synthetic construct
  source 1..1458: mol_type = other RNA
atggccctccccgtcaccgccctgctcctccccctggcactcctgctgcacgccgccaggcccgacatccagatgacccagaccaccagcagcctcagtgcctccctgggagatagggtgactatctcctgcagggccagtcaggacatcagcaagtacctgaattggtaccagcagaagcctgacggcactgtgaagctgctgatctaccacacctcccgcctgcacagcggtgtgcccagccggttctccggctctgggtccgggactgactacagcctgaccatctccaacctggagcaggaggacatcgccacctacttctgccagcaggggaacaccctgccatacacctttggtggcgggaccaagctggagatcaccggcggaggcgggagcggcgggggcggctccggcggaggcgggagcgaggtcaagctgcaggagtctggccccggcctggtggcccccagccagagcctgagcgtgacctgcaccgtctccggcgtgagcctgcccgactatggcgtgagctggatcaggcagccacccaggaagggcctggagtggctgggcgtgatctgggggtctgagaccacctactataacagcgccctcaagtcacgcctgaccatcatcaaggacaacagcaagtcccaggtgttcctgaagatgaacagtctgcagacagacgacaccgccatctactactgtgccaagcactactactatggcgggtcttacgccatggactactgggggcaggggacgagtgtgactgtgtccagcacaaccacacccgcccctaggcccccaacacccgcccccactatcgcaagccagcccctgagtctgaggcccgaggcctgccggcccgccgccggcggcgccgtgcacaccaggggcctggacttcgcttgcgatatctacatctgggctcctctggccggaacctgcggcgtgctgctgttgtccttggtgatcactctgtactgcaagaggggccgcaagaagctgctgtacatctttaagcagcctttcatgcggcccgtgcagaccacccaggaggaggacggctgcagctgcaggttcccagaggaggaggagggcgggtgtgagctgagggtgaagttctccaggtctgccgacgcccccgcctaccagcagggccagaatcagctctacaatgagctgaatctgggccgccgggaggagtacgatgtgctcgacaagcggcgggggcgcgacccagagatgggcggcaagcctaggagaaagaaccctcaggaggggctctacaatgagctgcagaaggacaagatggctgaggcgtattcagagatcgggatgaaaggggagaggaggaggggcaaggggcacgacggcctgtatcaggggctgagcaccgccaccaaggacacctatgacgcactgcacatgcaggccctgcccccgcgg

SEQ ID NO: 142 - RNA, 1458 nt, synthetic construct
  source 1..1458: mol_type = other RNA
atggccctgcccgtgaccgccctgcttctgcccctggccctgctgctccacgccgccaggccagacatccagatgacccagacaacctccagcctgagcgccagcctgggcgacagagtgaccatcagttgccgggccagccaggacatcagcaagtacctgaattggtaccagcagaagcccgacggcaccgtgaagctgctgatctaccacaccagccgcctgcacagcggcgtgcccagcaggttcagcggcagcggctccggcaccgactacagcctgaccatcagcaacctggagcaggaggacatcgccacctacttctgccagcagggcaataccctgccctacacctttggcggcggcaccaagctggagatcaccggcggcggcggaagcggcggcggcggctccggcgggggcggcagcgaggtgaagctgcaggagagcggcccaggcctggtggcccccagccagagcctgagcgtgacctgcaccgtgagcggcgtgtccctgcctgactacggggtgagctggatcaggcagccccccagaaagggcctggagtggctgggcgtgatctggggcagcgagaccacctactacaattccgccctgaagtcccggctgaccatcatcaaggacaattccaagagccaggtgttcctgaagatgaactccctgcagaccgacgacaccgccatctactactgcgccaagcactactactacggcggctcctacgccatggactactggggccagggcacctccgtgaccgtgagctctaccaccacccccgcccctcggcccccaacacctgcccccaccatcgctagccagcccctgtctctgagacccgaggcttgcagaccagccgccggcggcgccgtgcacaccagaggcctggacttcgcctgcgacatctacatctgggcccccctggccggcacctgcggcgtgctgctgctgagcctggtgatcaccctgtactgcaagaggggaaggaagaagctgctgtacatctttaagcagcctttcatgagacccgtccagaccacacaggaggaggacggctgtagctgcaggttcccagaggaggaggagggcggctgcgagctgcgggtgaagttcagcaggtccgctgacgcccccgcctaccagcagggccagaaccagctgtacaatgagctgaacctgggccggagagaggagtacgacgtgctggacaagaggagaggcagggaccccgagatgggcggcaagcctagaaggaagaacccccaggagggactgtacaacgagctgcagaaggacaagatggccgaggcctactctgagatcggcatgaagggcgagaggagaagaggcaagggccacgatggcctgtaccagggcctgagcaccgccaccaaggacacctacgacgccctgcacatgcaggccctgcccccccgg

SEQ ID NO: 143 - RNA, 1458 nt, synthetic construct
  source 1..1458: mol_type = other RNA
atggcattaccagttaccgccctcctcctccccctcgccctcctcctccacgccgcccggccagatatccagatgacccagaccacctcctccctcagcgcctccctcggcgaccgggtgaccatctcttgccgggccagccaggacatcagcaagtacctcaattggtaccagcagaagccggacggcaccgtgaagctcctgatctaccacacctcccgcctgcacagcggggtgcccagcaggttcagcggcagcggcagcggcaccgactactccctgaccatcagcaacctggagcaggaggacatcgccacctacttctgccagcaggggaacaccctgccctacaccttcggcggcggcaccaagctggagatcaccgggggcgggggcagcgggggcgggggcagcggcgggggcggcagcgaggtgaagctgcaggagagcggcccaggcctggtggcccccagccagagcctgtccgtgacctgcaccgtgagcggcgtgtccctgcccgactatggcgtgagctggatccgccagccacccaggaagggcctggagtggctgggcgtgatctggggcagcgagaccacctactacaacagcgccctgaagtccaggctgaccatcatcaaggacaacagcaagagccaggtgttcctgaagatgaacagcctgcagaccgacgacaccgccatctactactgcgccaagcactactactacggcggcagctacgccatggactactggggccagggcacctccgtgaccgtgagcagcaccaccacccccgccccccgcccccccacccccgcccccaccatcgccagccagccgctgagcctgcggccggaggcctgcaggcccgccgccggaggcgccgtgcacacccggggcctggacttcgcctgcgacatctacatctgggcccccctggccggcacctgcggcgtgctgctgctgagcctggtgatcaccctgtactgcaagcggggcaggaagaagctgctgtacatcttcaagcagcccttcatgcggcccgtgcagaccacccaggaggaggatggctgcagctgcaggttccctgaggaggaggagggcggctgcgagctgcgggtgaagttctcccgcagcgccgacgcccccgcctaccagcagggccagaatcagctgtacaacgagctgaatctgggccggcgggaggagtacgacgtgctggacaagcgccggggccgggaccccgagatgggcggcaagccccggcgcaagaacccccaggagggcctgtacaacgagctgcagaaggataagatggccgaggcctacagcgagatcggcatgaagggcgagcggcggagggggaagggccacgacggcctgtatcagggcctgtccaccgccaccaaggacacctacgacgccctgcacatgcaggccctgcccccccgg

SEQ ID NO: 144 - RNA, 1458 nt, synthetic construct
  source 1..1458: mol_type = other RNA
atggcattacccgtgacggccctcctactccccttagctctcttgctccacgccgcccgcccggacatccagatgacccagacgacctcgagcttgagtgcgtcactcggggaccgagtgacgatctcttgccgagcgtcgcaggacatctcgaagtacttgaattggtaccagcagaagcccgacgggaccgttaagctattaatctaccacacgagtagattgcatagcggcgttcccagtcggttctctggtagcggttcaggtactgactactcgctgaccatcagcaacctggagcaggaggacatcgccacctacttctgccagcagggtaacacgcttccctacaccttcggtggggggaccaagctggagatcactggtggcggaggtagcggtggcggtggttcgggcgggggcgggagcgaggtgaagctgcaggagagtgggcctggcctcgtcgcgccgtcacagagcctctctgtgacgtgcaccgtgagcggggtcagcctgccggactacggagtgtcctggatccggcagccgccccgcaaaggcctcgagtggctcggggtcatttgggggagcgagacaacgtactacaatagtgcgttgaagtcgcgacttactattatcaaggataatagtaagtcgcaggtctttctgaagatgaactcacttcagactgacgacaccgcaatatactattgcgccaagcactactactatggaggcagttacgcgatggactactgggggcaggggacatccgtgactgtctccagcaccaccacgccggccccgcggccccccacccccgcgccgacgatcgcgtctcagcccctcagcctgaggcctgaggcgtgtcgtccagcggcgggtggggccgtgcacacacggggactagactttgcatgcgacatctacatctgggccccgctggcggggacctgtggagtgttgctgctgagtctagtcatcaccttgtactgcaagcgcggccgtaagaagctcctgtacatcttcaagcagcccttcatgaggcctgttcaaacgacgcaggaggaggacgggtgcagctgccgcttccctgaggaggaggagggcggctgcgagctgagagtgaaattctcacgctcggcggacgccccagcttatcagcagggccagaaccagctgtacaatgagctgaacttaggccgcagggaggagtacgacgtgctcgacaagcgccggggtcgtgacccggagatgggtggcaagccccggcgcaagaatccgcaggaggggctctacaacgagctccagaaggacaagatggcggaggcctactccgagattggcatgaagggggagcgccgtcgtggtaagggccacgacggcctgtaccagggcctgtcgacggccactaaggacacttacgacgccctgcacatgcaggcgttgccgccccgg

SEQ ID NO: 145 - RNA, 1458 nt, synthetic construct
  source 1..1458: mol_type = other RNA
atggcattaccagttacagccctgttgctgcccctggccctgctgctgcacgccgcccggccagatatccagatgacgcagaccaccagctccctgtcagcctccctgggggacagggtgaccatcagctgccgggccagccaggacatcagcaagtacctgaactggtaccagcagaagcctgatggcaccgtgaagctgctgatctaccacacctcccgcctgcacagcggggtgcccagcaggttcagcggcagcgggtcaggcaccgactacagcctgaccatctccaacctggagcaggaggacatcgccacctacttctgccagcaggggaacaccctgccctacaccttcggcgggggcaccaagctggagatcactggcggcggggggagcgggggcggggggtctgggggcgggggtagtgaggtgaagctgcaggagagcggaccaggcctggtggcccccagccagagcctgtccgtgacctgcaccgtgtctggcgtgagcctgccagactacggcgtgtcctggatccgccagccacccaggaagggcctggagtggctgggcgtgatctggggcagcgagaccacctactacaacagcgcgctgaagtccaggctgaccatcatcaaggacaacagcaagagccaggtgttcctgaagatgaacagcctgcagaccgacgacaccgccatctactactgtgcgaagcactactactatggcgggtcctacgccatggattactggggccaggggacctcagtgaccgtgagcagcaccactacccccgcccccagaccccccacccccgctcccaccatcgccagccagcctctgagcctgcggccagaggcctgcaggcctgccgccgggggggccgtgcacacccgcgggctggacttcgcctgcgacatctacatctgggcccccctggccggcacctgcggggtgctgctgctgagcctggtgatcaccctgtactgcaagcggggccggaagaagctgctgtacatcttcaagcagcccttcatgcgccccgtgcagaccacccaggaggaggacggctgcagctgccgcttccctgaggaggaggagggcggctgcgagctgcgggtgaagttctcccgcagcgccgacgcccctgcgtaccagcagggccagaatcagctgtacaacgagctgaatctgggcaggcgggaggagtacgacgtgctggacaagcgccggggccgggaccccgagatggggggcaagccccggcgcaagaacccccaggaggggctgtacaacgagctgcagaaggataagatggccgaggcctattccgagatcggaatgaagggcgagcggcggcggggcaaggggcacgacgggctgtaccagggcctgagcaccgccaccaaggacacctacgatgccctgcacatgcaggccctgcccccccgg

SEQ ID NO: 146 - RNA, 1458 nt, synthetic construct
  source 1..1458: mol_type = other RNA
atggctctaccagtgacagccctcctcctcccactcgccctcctcttgcacgccgcgcgcccagatatacagatgacccagacgacgtccagcttgagtgcgtcactcggggaccgagtgacgatctcttgccgggcgtcgcaggacatctccaagtacctcaattggtaccagcagaagccagacgggacggtgaagctcctgatctatcacacgtcccgtctgcattctggtgtcccgagccggttctccggctcgggaagtgggactgactacagtctcacgatatccaacctggagcaggaggatatcgcaacgtatttctgccagcagggaaatacgttgccgtacaccttcgggggagggacaaagctggagatcactggtgggggcgggagtggggggggggggagcgggggagggggcagcgaggtcaagctccaggagtctgggccgggcctcgttgccccctcccagtcactctcagtcacttgcaccgtttccggggtgtcgctgccggactacggggtgtcgtggatccggcagccaccccggaaagggctggagtggctgggagtgatctgggggtccgagactacgtactacaatagtgcgttgaagtctcggctcaccataataaaggacaatagtaagtcgcaggtctttctgaagatgaacagtcttcagacggacgacactgcgatttactattgtgccaagcactattattatggtgggtcgtacgccatggattactggggtcaggggacctcagtgaccgtgagcagtacgaccacccccgctccccgcccccccactcccgcccccaccatagcatcccagcctttgtccctccgcccggaggcgtgccgccccgccgcgggcggggcggtgcacacgcgagggctggacttcgcgtgtgacatctatatctgggcgccgctggcggggacgtgcggagttcttctgctctcgcttgtaatcacgctgtattgcaagcgagggcggaagaagctcctgtacatcttcaagcagccgttcatgcggccggtccagacgacccaagaggaggacgggtgctcctgccggttcccggaggaggaggaggggggctgtgagctgcgggtcaagttcagccgttcagctgatgcccctgcgtaccagcaggggcagaatcagctgtacaacgagctgaacttgggccgtcgagaggagtacgacgtcctcgacaagcgtcgaggacgtgatcccgagatggggggtaagccccgccgtaagaatcctcaggaggggctctacaacgagctccagaaggataagatggcggaggcttactccgaaatcgggatgaagggagagcgccgtcgtggcaagggccacgacgggctctaccagggcctctcgacggccaccaaggacacctacgacgccctgcacatgcaggcgttgccgccgcgg

SEQ ID NO: 147 - RNA, 1458 nt, synthetic construct
  source 1..1458: mol_type = other RNA
atggccctacccgtgaccgctctgctgctacccctggctctgttactgcacgccgcccgccctgacattcagatgactcagactaccagcagcctgagcgcctcgttgggagaccgcgttaccatctcctgtcgcgctagccaggacatatctaaatatctcaattggtaccaacagaagcctgacggcacagttaaacttctcatttatcatacctcacgcctccattccggagtgcccagtaggttcagtggaagcggcagtggaaccgactatagcctcactattagtaacttggaacaagaggacatagcaacatatttttgccaacaggggaataccttgccatatacattcggtgggggaactaaactcgagatcaccggcggaggtggtagcgggggaggaggatcagggggaggaggtagtgaggtgaagttgcaggagtcaggtccgggactggtcgcgccatcccagtcactttcagtgacatgtacagtgagtggtgtttcactgcccgactacggagtctcctggatccgccagcctccccgaaagggattggagtggcttggggtaatatgggggagcgaaacgacctactataactcagccctcaaaagtaggctgacaattataaaggataacagtaaaagtcaagttttccttaaaatgaacagcctgcagacagatgatacggccatctactactgtgcgaaacattactactacgggggctcctatgccatggattattggggacagggaacctcagtcactgtgtcatctacaaccactccggctccacggcctcctacacccgcacccactattgcctcacagcctctcagcctgagacctgaagcctgcagacccgctgcgggcggcgccgtacacacccgaggtctggattttgcctgtgacatttatatctgggccccgttggctggaacatgcggtgtattattgctgtccctggtgataactttgtactgtaagcggggaaggaagaagctactgtacatcttcaagcagccctttatgcgccctgtgcaaacaactcaagaggaggatggttgttcgtgcaggttcccagaggaggaggaaggcgggtgtgagttgcgtgttaagttttcgagatcagctgacgcccctgcttaccagcaagggcagaaccagctctacaacgagctgaatcttggacgtcgagaagagtacgatgttttggataaaaggcgtgggcgcgatccagagatgggcggtaaaccaaggcgtaagaatcctcaggagggtctctataacgagttgcagaaggataagatggctgaggcttacagcgaaatcggaatgaaaggagagagacggcgaggtaaaggtcacgatggtctctatcagggattgagcacagcgaccaaggacacgtacgacgcccttcacatgcaagcgctgcctccgagg

SEQ ID NO: 148 - RNA, 1458 nt, synthetic construct
  source 1..1458: mol_type = other RNA
atggctctgccagtaaccgcgcttctactgccattagccttgctgctccacgcagcccgacccgacattcagatgactcagactactagctctctgagtgcatcgctaggtgatcgtgttacgatttcctgccgggcctcacaggatatttctaaatacctgaactggtaccaacagaagcccgatgggacggtgaagttattgatctaccacaccagtcggttgcactccggcgtcccatccaggttttccgggtcaggcagcggtactgactatagccttaccatcagcaacctggaacaggaagacatagccacctacttctgccagcaaggcaacacactgccctacacctttggtggcggaaccaagctggagattactgggggcgggggttctggtggtggtggttcgggcgggggagggagtgaggtcaagcttcaggaatcgggacctggtctagtggctccatctcagtccctgtccgtgacttgcactgtgtccggcgtgtcgttgccggactacggtgtctcgtggatcaggcaacccccccgcaagggcctcgagtggctaggggtgatttggggctccgaaactacctactacaactccgcgctcaagtctaggctgacgatcatcaaggacaactctaaatcccaagtgttcttaaagatgaacagtctgcaaacagacgataccgcgatatattattgcgctaagcattattattatggtggctcctacgccatggattactggggtcaaggcacttccgttacagtcagttcaacaacaaccccagctccacgtcctccaacaccagccccgaccatcgcctctcagccactgtccctcaggcctgaggcctgccggcctgcggccggaggggcagtgcatacgcgggggttggactttgcttgcgacatttatatctgggcgcccctggccggtacatgtggagtgctccttttgtccctggtgatcaccttgtactgcaagagagggagaaagaagctactctacatctttaagcagccgttcatgcgaccggtccagaccacccaggaagaggacggctgcagctgccggtttcctgaagaggaagaaggcggctgcgaattacgcgtgaagttttcgcgcagcgccgatgctcccgcttatcagcaaggccaaaaccagctctacaatgagttgaacctggggcgacgtgaggagtatgatgttcttgataagcggcggggccgagaccctgagatgggcgggaaaccgcgacgtaagaatcctcaggaggggctgtacaacgagctccagaaagataagatggcggaagcgtatagcgagatcggtatgaagggggagcgccggcgcgggaagggtcacgatggactgtaccagggcctcagtaccgcgacaaaggatacctatgacgcgttgcatatgcaggccctgcccccaagg

SEQ ID NO: 149 - RNA, 1458 nt, synthetic construct
  source 1..1458: mol_type = other RNA
atggccctgcctgtgaccgccctgctgctgccactggccctgctgctgcacgccgcccggccagatatccagatgacacagacaacctccagcctgagcgccagcctgggggacagagtgaccatcagctgccgggcctcccaagacatcagcaaatacctgaactggtaccagcagaaacccgatggcaccgtgaagctgctgatctaccacacttccaggctgcacagtggggtgccctcccggttctccggcagcggcagcggcaccgactacagcctgaccatcagcaacctggagcaggaggacatcgccacctacttttgccagcagggcaacaccctgccctacaccttcggcggcggcaccaagctggaaatcaccgggggcgggggatctgggggcggcggctccggcggcggaggatccgaggtgaagctgcaggagtccggcccaggcctggtggcccccagccaaagtctgtccgtgacctgtaccgtgtccggcgtgtccctgcccgactacggcgtgagctggattcgccagcccccccggaagggcctggagtggctgggcgtgatctggggcagcgagaccacctattacaacagcgccctgaagtccaggctgaccatcattaaagataatagcaagagccaagtgttcctcaagatgaatagcctgcagaccgacgataccgccatctactactgtgccaaacactactactacggcgggagctacgccatggactactggggccaaggcacctccgtgaccgtgagcagcaccaccaccccagcccccaggccccctacccccgctcctacaattgccagccagccactgtccctgcgccctgaggcctgccggcccgccgccggcggcgccgtgcatacccggggcctggacttcgcctgtgacatctacatctgggcccccctggctgggacctgcggcgtgctgctgctgagcctggtgatcacactgtactgcaaaaggggccggaaaaaactgctgtacatcttcaagcagcccttcatgagacccgtgcagaccacccaggaggaggacggatgctcctgccgcttcccagaggaggaggagggcgggtgcgagctgagagtgaagtttagccgcagcgccgatgctcccgcctaccagcagggccagaatcagctgtataacgaactcaacctgggcagaagagaagagtacgatgtgctggataaaagacggggcagggaccccgagatgggcggcaagccaagaagaaagaatccccaggagggcctgtacaacgagctgcagaaggataagatggccgaagcctacagcgagattggcatgaagggagagcggaggcgcggcaagggccacgacggcctctaccagggcctgtccaccgccaccaaggatacctacgacgctctgcacatgcaagctctgcccccccgc

SEQ ID NO: 150 - RNA, 1458 nt, synthetic construct
  source 1..1458: mol_type = other RNA
atggccctgcccgtcaccgccctgctgctgcccctggccctgctgctgcacgccgcccggcccgatatccaaatgacacagaccaccagctccctgagcgccagcctgggcgacagagtgaccatctcctgcagggctagccaggacatcagcaagtatctgaactggtaccagcagaagcccgacggcaccgtgaagctcctgatctatcacacctccaggctgcacagcggcgtgcccagcagattctccggcagcggcagcggcaccgactactccctgaccattagcaacctggagcaagaggacatcgccacctacttctgccagcagggcaacacactgccctatacctttggcggcggcaccaagctggagatcaccggcggcggcggcagcggcggcggcggcagcggcggcggcggctccgaggtgaaactgcaggaaagcggccccggcctggtggccccctcccagagcctgagcgtgacctgcaccgtgagcggcgtgagcctgcctgactacggcgtgtcctggattaggcaaccccccaggaagggactggagtggctcggcgtgatttggggaagcgagaccacctactacaactccgccctgaagtccagactcaccatcattaaagataacagcaaatcccaagtgttcctgaagatgaactccctgcaaaccgacgacaccgccatctactactgcgccaagcactactactacggaggcagctacgccatggactattggggccagggcaccagcgtgaccgtgagcagcactacaacccccgcccccagacctcctacccctgcccccaccatcgcctcacagcctctgagcctgagacccgaggcctgcaggcccgccgccggcggcgccgtgcacacccggggactggacttcgcctgcgacatttacatctgggcccccctggcaggcacctgcggcgtgctcctgctgagcctggtgattaccctgtactgcaagagaggaagaaagaaactgctgtacatcttcaagcagcccttcatgaggcccgtgcagaccacccaggaagaggacggctgctcctgtaggttccccgaggaggaggagggcggctgcgagctcagagtgaagtttagcaggagcgccgacgcccccgcttaccagcaaggccagaaccagctgtacaatgaactgaacctgggcagacgggaggaatacgacgtgctggacaaaaggagaggaagagaccccgagatgggcggaaagccccggcgcaagaacccacaggagggactgtacaacgagctgcaaaaagacaagatggccgaagcttacagcgagatcggaatgaagggcgagaggagacgcggcaagggacacgatggcctctaccagggcctgagcaccgccaccaaagacacctatgatgccctccacatgcaagccctgccccctagg

SEQ ID NO: 151 - RNA, 1458 nt, synthetic construct
  source 1..1458: mol_type = other RNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 152 - RNA, 1458 nt, synthetic construct
  source 1..1458: mol_type = other RNA
atggcacttcccgttaccgccctcctccttccactcgccctcctcctccacgccgcccgcccagatatacagatgacacagaccacctccagcttgagtgcctccctcggggacagggtgaccatctcttgtcgggcttctcaggacatcagcaagtacctcaattggtaccagcagaagcccgacgggaccgtgaagctcctgatctaccacaccagcaggctgcacagcggcgtgccctccaggtttagcgggtcagggagtgggaccgactactccctgaccatctcaaacctggagcaggaggacatcgccacctacttttgccagcagggcaacaccctgccctacaccttcgggggaggcactaagctggagatcactggcggtggtgggagcgggggtggggggtctgggggcgggggtagcgaggtgaagctgcaggagtccggacccggcctggtcgcccccagccagtccctgtccgtgacatgcaccgtgtctggcgtgagcctgccagactacggcgtgtcatggatcaggcagcccccccggaaggggctggagtggctgggggtgatctgggggtccgagactacctactacaacagcgcgctgaagtccaggctgaccatcatcaaggacaactccaagagtcaggtgttcttgaagatgaacagcctgcagaccgatgacaccgccatatactactgtgcgaagcactactactatggcgggtcatacgccatggattactggggccaggggacctcagtgaccgtgagcagcaccaccacccccgcccccagaccccccacccccgctcccaccatcgccagccagcccctgagcctgcgccccgaggcctgccggcccgcggccggcggcgccgtgcacactcgggggctggacttcgcctgtgacatctatatctgggcccctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggttcccagaggaggaggagggcgggtgcgagctgcgggtgaagttctcccgcagcgccgatgcccctgcgtaccagcaggggcagaatcagctctacaacgagctgaacctgggccgccgcgaggagtacgacgtgctcgacaagcggcggggccgggatcccgagatggggggtaagcctcggcggaagaatcctcaggaggggctctacaacgagctccagaaggataagatggccgaggcttactccgagatcgggatgaagggggagcggcggcggggcaaggggcacgacggcctgtatcagggcctgagtaccgccaccaaggatacttacgacgccctgcacatgcaggccctgcccccccgg

SEQ ID NO: 153 - RNA, 1458 nt, synthetic construct
  source 1..1458: mol_type = other RNA
atggccctaccagtcaccgccctcctcctccccttggccctcctcctccacgccgcccggccagatattcagatgacacagaccacctccagccttagtgcctccctcggagatagggtgaccatttcttgtcgggcttctcaggacatcagcaagtacctcaattggtaccagcagaagcccgacgggacagtgaagttgctgatctaccacactagcaggctgcatagtggcgtgccctcccgcttctctggaagcgggagcggcaccgactacagcctgactatcagcaacctggagcaggaggacatcgccacctacttctgccagcaggggaataccctgccctataccttcgggggaggcactaagctggagattactggcggaggcggcagtggaggaggagggtcagggggaggaggatctgaagtgaaacttcaggagagtgggcccggccttgttgctccttctcagagcctgtccgtgacatgtaccgtgtctggcgtgtctctgccagactacggcgtgtcatggatcaggcagcctcctaggaaggggctggagtggctgggcgtgatctggggtagtgagactacttactacaatagtgccctgaagtcccggctgactattatcaaggataatagcaagtctcaggtgttcctgaagatgaacagcctgcagacagatgacacggccatctactactgtgcgaagcactactactatggcggcagctacgccatggattactggggccagggcactagtgtgacagtgagtagtaccactaccccagctccacgcccacctactccagcccctacaattgcctcccagcctctgtctctgcgccctgaagcttgtcgccctgctgctggtggagctgtgcacaccagagggctggacttcgcctgcgacatctatatctgggctcctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggtttccagaggaggaagagggtggatgtgagctgcgggtgaagttcagccgctctgctgatgcaccagcctaccagcagggccagaatcagctctacaacgagctgaatctgggccgcagggaggagtacgacgtgctcgacaagcgcaggggcagagatccagagatgggaggcaaacctaggaggaagaatccccaggaggggctctacaacgagctgcagaaggacaagatggccgaggcctactctgagatcggaatgaagggcgagaggaggcggggaaaaggccacgatggcctgtatcaggggctgtccaccgccaccaaggacacctatgatgccctgcacatgcaggccctgcctcctcgg

SEQ ID NO: 154 - RNA, 1458 nt, synthetic construct
  source 1..1458: mol_type = other RNA
atggctctcccagtgaccgccctcctccttccccttgccctcctcttgcacgccgctagacccgacatccagatgactcagactacctctagtttgagtgcatccttgggtgatcgggtcaccatctcttgtcgggcttctcaggatatcagtaagtaccttaattggtatcagcagaagcccgacgggaccgtgaagctcttgatctaccatacatcaaggcttcacagcggcgtgccctccaggtttagcgggtcagggagtgggaccgactactccctgaccatctcaaacctggagcaggaggacatcgctacttacttctgccagcagggtaacaccctgccctacacctttgggggagggacaaagctggagatcactggtgggggcgggagtggggggggggggagcgggggtgggggcagcgaggtcaagctccaggagtctgggcctggcctcgttgccccatcccagagtctgtcagtgacatgcactgtgagtggcgtgtcactgccagactatggggtgtcctggatccgccagccacccaggaagggcctggagtggctgggcgtgatctgggggagtgagaccacctactataatagtgctttgaagtctagacttactattatcaaggataatagtaagtctcaggtctttctgaagatgaacagtcttcagacagacgacaccgcaatatactattgcgcaaagcactattattatggggggtcttacgccatggactactggggtcagggaacgtccgtgaccgtcagtagtaccaccacccccgctccccgcccccccactcccgcccccaccattgcatcccagcctttgtccctccgcccagaggcctgtaggccagccgccgggggtgcggtgcacacccgggggctggacttcgcatgtgacatctacatatgggctcctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggttcccagaggaggaggagggggggtgtgagctgagggtgaagttttcacgctcagctgacgcccccgcctaccagcagggccagaatcagctctacaacgagctgaatctgggccggcgggaggagtacgatgtcctcgacaagaggagggggagggatcccgagatggggggtaagcctcggcggaagaatcctcaggaggggctctacaacgagctccagaaggataagatggccgaggcttactccgagatcgggatgaagggggagcggcggagggggaagggccacgatggactgtatcaggggttgtccactgccacaaaggacacctatgatgcactccatatgcaggccctgccccctcgg

SEQ ID NO: 155 - RNA, 1458 nt, synthetic construct
  source 1..1458: mol_type = other RNA
atggctctcccagttacagccctcttgttgcctttggccttgctcttgcacgccgcaaggcccgatatccagatgacccagaccaccagttccctaagcgccagcttgggggaccgggtgaccatctcttgtcgggccagtcaggatatcagcaagtacctcaattggtaccagcagaagccggacggcaccgtgaagttgctgatctatcacaccagtaggctgcactccggcgtgccctcccggttctccgggagcggcagcgggacagactacagtctgaccatcagcaacctggagcaggaggacattgccacctacttctgccagcaggggaacaccctgccctatacatttggcgggggcaccaagctggagattacaggcgggggaggctctggcggcgggggcagcggggggggcggctctgaggtgaagcttcaggagtccggccccggcctcgttgcccccagccagagcctctccgtgacctgtaccgtctccggcgtgtccctgccagattatggggtgtcctggatccgccagccaccccggaaggggctggagtggctgggcgtgatctggggctcagagacaacctactacaactctgccctgaagagtagacttactattatcaaggataatagtaagtctcaggtgtttctgaagatgaatagcctgcagacagatgacacggccatctactactgcgctaagcactactactatggcggcagctatgccatggactactggggccaggggactagcgtgacagtgtcctccaccaccacccctgctcccaggccccccacgcctgcccccaccatcgcgagccagccactgtccctccggccagaggcctgccggcctgctgccggaggggcagtgcacacaagagggctggatttcgcctgtgacatttacatctgggctcctctggccggcacctgcggcgtcctcctcctgagcctggtgatcaccctctactgcaagaggggcaggaagaagctgctgtacatcttcaagcagcctttcatgaggcccgtgcagaccacccaggaggaggacggctgcagctgccggttcccagaggaggaggagggcggctgcgagctgcgggtgaagttctcccgcagcgcagacgcccccgcctaccagcagggccagaatcagctgtacaacgagctgaatctgggccggcgggaggagtacgacgtgctggacaagcgccggggccgggaccccgagatggggggcaagccccggcgcaagaatcctcaggaggggctgtacaacgagctccagaaggataagatggcggaggcctacagcgagatcgggatgaagggcgagcgcaggcggggcaaggggcatgacgggctgtaccagggcctgtcaactgccaccaaggacacctacgacgccctgcacatgcaggccctgcctccccgg

SEQ ID NO: 156 - RNA, 1458 nt, synthetic construct
  source 1..1458: mol_type = other RNA
atggccttaccagttacggcgctattgctcccgttggcgctattgctccacgccgcgcgcccggacatacagatgacgcagacgacgtcttcgttatcggcgagtttgggcgaccgggttacgatatcgtgcagggcttcgcaggacataagtaagtaccttaattggtaccagcagaagcccgacggtacggtaaaactactcatctatcatacgtcgcgtttgcatagtggagtaccctcccgtttttcggggagtgggtcgggtaccgactattcgctaacgatatcgaacttggagcaagaagatatcgcgacgtacttttgccagcaagggaatacgttaccgtacacgtttggtggaggtacgaaactagagatcacgggtggtggtggttcgggtggtggtggttcgggtgggggcggctcggaagtgaaacttcaagagtcgggtcccggtctagtggcgccgagccaatcgctatcggtaacgtgtacggtttcgggtgtatcgctaccggattacggtgtatcgtggatacgccaaccgccgcgtaagggcctagaatggctaggggttatatggggtagtgagactacctattataactcggcgttgaaatcgcgtctaacgataataaaagataattcaaaatcgcaggtttttctaaaaatgaattcgctacaaacggatgatacggcgatatattattgtgcgaagcactattattatggtggttcctatgcgatggattattggggtcaaggtacgtcggtaaccgtgtcgtcgacgacgacgccggcgccgcgcccgccgacgccggcgccgacgatagcgtcgcaaccgctatcgttgcggccggaggcgtgccgcccggcggcgggcggggcggtgcatacgcgcggcctcgattttgcgtgtgacatttacatatgggcgccgctagcgggcacgtgtggtgtcctactactatcgttggtgatcaccctatattgtaagcgtggcaggaagaagcttctttatatatttaaacagccgtttatgcggccggtacaaacgacgcaagaagaagatggttgttcgtgccgcttcccggaagaagaagaaggtggctgtgagctacgcgtaaagttttcgcgttcggcggatgctccggcgtatcaacaaggtcaaaatcagttgtataacgaactgaatctagggcgtcgtgaagaatacgacgtcctagataaacgtcgtggtcgggacccggaaatgggtggtaagccgcgtcgtaaaaacccgcaagaggggttatataatgaactccaaaaggataaaatggcggaggcgtattcggagatcgggatgaaaggcgagcgtcgccgtggtaagggccacgacggcctttatcaaggtctctcgacggcgacgaaggatacgtatgatgcgttgcatatgcaagcgttgccgccgcgt

SEQ ID NO: 157 - RNA, 1458 nt, synthetic construct
  source 1..1458: mol_type = other RNA
atggcactaccggtaacggcgctactactaccgctagcgttactattgcatgcagcacgtccggatatacaaatgacgcaaacaacttcgagtttatcggcatctttaggagaccgtgtaaccatatcgtgtcgagcgtctcaagacatatctaagtatttaaattggtatcaacaaaaacctgacggcacggtaaaattactaatataccatacgtcgcgactccattcgggtgtaccatcgcgtttttcgggatcgggttcgggtactgattactcgcttactatatcgaatttagaacaagaagatatcgcgacttatttttgccaacaagggaacacgctaccttatacctttggtggagggaccaaattggaaataacgggggggggcgggagtggaggaggaggttcagggggggggggttcagaagttaaattacaagaatcaggaccaggattagtagcgccgtcgcaatcgttatcagttacgtgtacggtctcgggtgtaagtctaccggattacggagtatcgtggattaggcaacccccgcgcaaggggcttgaatggcttggcgttatctggggctcggaaactacgtattataactcggctctaaaatcacgtttgacgataataaaagacaattcgaaatcgcaagtattcttaaaaatgaacagcctacaaacggacgacacggcgatatactattgtgcgaaacattattattatggtggtagttatgcgatggattactggggccaaggaacaagcgttaccgtatcgtccactacgacgcctgcgccgcgaccgcccacgccggcgcctaccatagcttcacaaccattaagcctccgtccagaggcgtgccgtcctgcggcgggtggtgctgtacatacgaggggtctagactttgcgtgtgatatttatatatgggctccattagcggggacatgcggggttctactactatcgctagtaataactctatactgcaaacgtggacgtaaaaagttactatatatatttaaacagccgttcatgagaccggtacaaactacgcaagaggaagatgggtgctcctgtagatttccggaggaagaagaaggtggttgcgaattaagagttaaattcagtcgaagtgcggacgcgccagcgtatcaacaagggcaaaatcaactatacaatgaactaaatcttggtcgaagagaagaatatgacgtattagacaaacggcgtggacgagaccctgagatgggtggaaaaccgcgaagaaaaaatccacaagaaggtttatacaatgaattacaaaaagataaaatggcggaggcatatagtgaaataggtatgaaaggagaacgaagacgtggtaaagggcatgacggattatatcaaggattgtcgacggcaacgaaagatacttacgacgcgttacatatgcaggccctaccgccgcga

SEQ ID NO: 158 - RNA, 1458 nt, synthetic construct
  source 1..1458: mol_type = other RNA
atggccttaccagtaacagccttattacttccacttgccctccttctacatgccgctagaccagatatccaaatgacacaaacgacctcaagcctttccgcgtctctcggtgacagggtaactattagttgtcgggcctcccaagatatttcaaaatacttaaattggtatcaacagaagccagatggtaccgtgaagctcctcatctaccacactagccgtcttcactctggagtaccaagcaggttttccggctccggaagtggtactgactacagtttgactataagcaacctagagcaagaggatattgccacatacttctgtcaacaaggaaacactctgccatatacattcggtggtggtacaaaactggaaataactggagggggaggttcaggcggcggcggatctggggggggtggcagtgaggtcaaattgcaggagtctggacccggtctggttgctccatcccaatcactttcagtgacctgcactgtatccggagtttctctgccagattatggagtgagttggatccgccagcctccacgcaagggccttgaatggctcggggttatatggggtagtgaaactacatactataatagcgctctgaaatctcgactcacaatcattaaggataatagcaaaagccaggtgtttttgaagatgaatagcctgcaaaccgatgataccgccatatattactgtgccaaacattactattacggaggctcctacgctatggattattggggacaggggacaagcgtgaccgttagctctactactacccccgcccctcgccctccaactccagctcccaccattgccagtcaaccactatccttaagacccgaggcatgcagacccgccgcagggggcgccgtacatacccgaggtttggattttgcctgcgacatctatatctgggccccccttgctgggacatgtggagtgttgctactctcgttggttatcactctgtattgcaagagaggaagaaagaaactattatatatttttaagcagccattcatgagaccggtgcagactacccaagaagaagacggatgttcatgtaggttccctgaagaagaggaaggaggctgcgaactgcgagtgaaattttccagatctgccgatgcaccagcttatcaacaaggacaaaaccagctatataatgaacttaatttagggagacgagaggagtacgacgtcctggacaaacgccgcggaagagaccctgagatgggaggtaagcccagaagaaaaaacccacaagaaggactgtacaatgaactccaaaaagacaaaatggctgaagcgtattccgaaattggcatgaaaggagaacggagaagaggtaagggtcacgacggcttatatcagggactctctacagcaaccaaagacacatatgatgcactacacatgcaggcactaccacccagg

SEQ ID NO: 159 - RNA, 1458 nt, synthetic construct
  source 1..1458: mol_type = other RNA
atggctctaccagtcacagcactactattgccgttggctctattgcttcacgcggcgcgaccggatatacagatgacccaaacgacgtcgagtctctcggcgtcgttgggtgatcgtgtaacgatatcgtgtcgtgcgtcgcaagatatctcgaaatatctaaattggtatcaacaaaaaccggacgggacggtgaaattattaatatatcacacgtcccgtctacattctggtgttccgagtcgatttagtgggagtggttcgggtacggactactccctaactatatcgaacttggaacaagaggatatagcgacgtacttctgccaacaaggtaatacgctaccgtatacctttggcggaggtacgaagctagaaataactggtgggggtgggagtggtggtggtggttcgggtggtggtggttctgaagtgaaacttcaagaatcgggaccggggctggtagccccgtcccaatcgttatcggtaacgtgcaccgtatcgggtgtatcgttaccggattatggtgtatcgtggatacgccaaccgccccgtaaaggcctagaatggctaggtgttatatggggttcggaaacgacgtactacaatagtgcgttgaaatcgcgacttactattatcaaggataatagtaagtcgcaagtatttttaaagatgaattcgttacagacggacgataccgcgatatactattgcgcgaagcactattattatggtggttcgtatgcgatggattattggggtcaaggtacgtctgtaacggtttcatctactactactccggcaccacgaccaccaactcctgctcctactatagcgtcgcaaccgttatcgttgcgacctgaagcatgtagaccagcggcaggtggtgctgttcatacgcgaggtctagacttcgcgtgtgatatatatatatgggccccgcttgcgggtacgtgtggtgtactattactatcgttagtaataacgctatactgcaagcggggccgaaaaaaattattatatatatttaaacaaccgtttatgcggccggtacaaacgacgcaagaggaagatgggtgttcttgtcgttttcctgaagaggaagaaggtggatgcgaactacgtgtaaaattttcgcgtagtgcggatgcgccggcctatcaacaaggccaaaatcaactctataacgagttgaatctaggacgtcgtgaagagtacgacgtcctagataaacgacgtggtcgtgacccggaaatgggtggcaaaccgcgtcgtaaaaacccgcaagagggtttatataatgagctacaaaaagataaaatggctgaagcgtactcggaaattggtatgaaaggtgaacgtcgtcgtggtaaaggccatgacggtttatatcaaggtctgagtacggcgacgaaggacacctatgatgctctacatatgcaagcgttaccgccgcgt

SEQ ID NO: 160 - RNA, 1458 nt, synthetic construct
  source 1..1458: mol_type = other RNA
atggctctacccgtcacggcgttattacttccgctcgcgttgctattacacgcagcgcgaccggatatacaaatgacgcaaacgacgtcgtcgttatcggcgtcgctaggagatcgtgtgacgatctcttgccgagcgtcgcaagatatctcgaaatatctaaattggtatcaacaaaaaccggacggtacggttaaattgttgatatatcatacgtcgaggctacattctggtgttccgagtcgatttagtgggagtggttcgggtacggactactccctaactatatcgaacttggaacaagaggatatagccacgtatttttgccaacagggcaataccctcccctacacgtttgggggagggacgaaactagaaattacaggtggtggtggttctgggggtggtggatcaggtggtggtggttctgaagtgaaacttcaagaatcaggaccgggtctagtcgcccccagtcaatcgctatcggtaacctgtacggtttctggtgtcagcttaccggattatggtgtaagctggatacgtcaaccgccccgtaaaggcctagaatggctaggtgttatatggggttcggagacgacgtactacaatagtgcgttaaaatcgcgacttactattatcaaagataatagtaagtcgcaggtttttttaaagatgaactctttacaaacggatgatacggcgatatactattgcgcgaagcactattattatggtggttcgtatgcgatggattactgggggcagggtacgtcggtaactgtgagttcgactacgactcctgctcctagaccgccgactccggcgccaactatagcatcgcagcctctatcgcttcgtccggaagcgtgtagaccagctgcaggtggtgctgtacatacgcgaggtctagacttcgcgtgtgatatatatatatgggcgcccctagcgggtacgtgtggggtgctattactatcgttagtaataacgctatattgcaaacgtggtaggaaaaagctactatatatatttaaacaaccttttatgcgtccggtacaaacgacgcaagaagaggatggttgtagttgtcgatttccggaagaagaagagggaggttgcgaactacgagtaaaatttagtcgatcggcggatgcgccggcgtatcaacaaggtcaaaatcaattatataatgaattaaatctaggacgtcgtgaagaatatgacgtcctagataaacgacgtggtcgtgacccagaaatgggtggtaaaccgcggcgtaaaaacccgcaagaaggtttatataatgagcttcaaaaagataaaatggcggaagcttattcggaaataggtatgaaaggtgaacgccgtcgtggtaaagggcatgacggtctttaccaaggtttgtcgacggcgacgaaagatacgtatgatgcgttacacatgcaagcgttaccgccgcgt

SEQ ID NO: 161 - RNA, 1458 nt, synthetic construct
  source 1..1458: mol_type = other RNA
atggcattacccgttaccgccctcctcttgcccctcgccctcttgctccacgccgcccggccagatatccagatgacccagaccacctcctccctcagcgcctccctcggcgaccgggtgaccatctcttgccgggcctcacaggacatcagcaagtacctcaattggtaccagcagaagcccgacggcaccgtgaagcttctgatctaccacacctcccggctgcacagcggggtgcccagcaggttcagcggcagcgggtcaggcaccgactacagcctgaccatctccaacctggagcaggaggacatcgccacctacttctgccagcaggggaacaccctgccctacaccttcggcgggggcaccaagctggagatcactggcggcggggggagcgggggcggggggtctgggggcgggggtagtgaggtgaagctgcaggagagcggaccaggcctggtggcccccagccagagcctgtccgtgacctgcaccgtgtctggcgtgagcctgccagactacggcgtgtcctggatccgccagccacccaggaagggcctggagtggctgggcgtgatctggggcagcgagaccacctactacaacagcgcgctgaagtccaggctgaccatcatcaaggacaacagcaagagccaggtgttcctgaagatgaacagcctgcagaccgacgacaccgccatctactactgtgcgaagcactactactatggcgggtcctacgccatggattactggggccaggggacctcagtgaccgtgagcagcaccactacccccgcccccagaccccccacccccgctcccaccatcgccagccagcctctgagcctgcggccagaggcctgcaggcctgccgccgggggggccgtgcacacccgcgggctggacttcgcctgcgacatctacatctgggcccccctggccggcacctgcggggtgctgctgctgagcctggtgatcaccctgtactgcaagcgcgggaggaagaagctgctgtacatcttcaagcagcccttcatgaggcccgtgcagaccacccaggaggaggatggctgcagctgcaggttccctgaggaggaggagggcggctgcgagctgcgggtgaagttctcccgcagcgcagacgcccccgcctaccagcagggccagaatcagctgtacaacgagctgaatctgggccggcgggaggagtacgacgtgctggacaagcgccggggccgggaccccgagatggggggcaagccccggcgcaagaatcctcaggagggcctgtacaacgagctgcagaaggataagatggccgaggcctacagcgagatcgggatgaagggcgagaggaggcgcggcaaggggcacgacggcctgtatcagggcctgtccaccgccaccaaggacacctacgacgccctgcacatgcaggccctgcccccccgg

SEQ ID NO: 162 - RNA, 1458 nt, synthetic construct
  source 1..1458: mol_type = other RNA
atggctctaccagttacagccctcttgctccctttggccttgttgctccacgcagcaaggccagacatccagatgactcagactacctctagtttgagtgcatccttgggagaccgggtgactatcagttgccgggcctcccaggacatcagcaagtacctcaattggtaccagcagaagccggacggcaccgtgaagttgctgatctaccacaccagcaggctgcacagcggcgtgccctcccggttctccgggagcgggtcagggaccgactactccctgaccatcagcaacctggagcaggaggacatcgctacttacttctgccagcaggggaacaccctgccctacacctttggcggagggacaaagctggagatcactggtgggggcgggagtggggggggggggagcgggggtggggggtctgaggtgaagctgcaggagtccggccccgggctggtcgccccctcacagagcctgtcggtgacatgcactgtgagcggggtgagcctgcccgactacggggtgagctggatccgccagcccccccggaagggcctggagtggctcggagtgatctggggctccgagaccacttactacaacagcgccctgaagtccaggttgactattatcaaggataatagcaagtctcaggtgttcctgaagatgaacagcctgcagaccgatgacaccgccatatactactgtgcgaagcactactactatggcgggtcatacgccatggactactggggccagggcacctcagtgaccgtcagctccaccaccacccccgctccccgcccccccactcccgcccccaccatcgcatcccagcccctgtccctccggccagaggcctgccggcccgccgcgggcggcgccgtgcacacccgcgggctggacttcgcctgcgacatctacatctgggctcctctggccggcacctgcggcgtcctcctcctgagcctggtgatcactctgtactgcaagcgcggaaggaagaagctgctgtacatcttcaagcagcccttcatgcgcccagtgcagaccacccaggaggaggacggctgcagctgccggttcccagaggaggaggagggcgggtgcgagctgcgggtgaagttctcccgcagcgccgacgcacccgcctaccagcagggccagaatcagctctacaacgagctgaatctgggccggcgggaggagtacgatgtgctggacaagcggaggggcagggatcccgagatggggggtaagcctcggcggaagaatcctcaggaggggctctacaacgagctccagaaggataagatggccgaggcttactccgagatcgggatgaagggggagaggcgccggggtaagggccacgatggcctttaccagggcctctccaccgcaacaaaggacacctatgatgccctgcacatgcaggccctgcccccccgg

SEQ ID NO: 163 - RNA, 1458 nt, synthetic construct
  source 1..1458: mol_type = other RNA
atggctttacccgttacagccctgttgctgccccttgccctgctgctgcacgccgcccggccagatatccagatgacgcagaccaccagctcattgtcagcctccctgggggatcgggtgaccatcagctgccgggccagtcaggacatcagcaagtacctgaactggtaccagcagaagcctgatggcaccgtgaagctgctgatctaccacaccagcaggcttcactccggcgtgccttcccgcttctctgggtccggctccggcacagactacagtctgaccatctccaacctggagcaggaggacatcgctacttacttctgccagcaggggaacaccctgccctacaccttcggcgggggcaccaagctggagatcactggcggtggtgggagcgggggtggggggtctgggggcgggggtagcgaggtgaagctgcaggagtccggacccggcctggtcgcccccagccagtccctgtccgtgacatgcaccgtgtctggcgtgagcctgccagactacggcgtgtcatggatcaggcagcccccccggaaggggctggagtggctgggggtgatctgggggtccgagactacctactacaacagcgcgctgaagtccaggctgaccatcatcaaggacaactccaagagtcaggtgttcttgaagatgaacagcctgcagaccgatgacaccgccatatactactgtgcgaagcactactactatggcgggtcatacgccatggattactggggccaggggacctcagtgaccgtgagcagcaccaccacccccgcccccagaccccccacccccgctcccaccatcgccagccagcccctgagcctgcgccccgaggcctgccggcccgcggccggcggcgccgtgcacactcgggggctggacttcgcctgcgatatctatatctgggcccctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggttcccagaggaggaggaggggggctgcgagctgcgggtgaagttctcccgcagcgcagacgcccccgcctaccagcagggccagaatcagctctacaacgagctgaatctgggccggcgggaggagtacgatgtcctggacaagcgccggggccgggacccagagatgggcgggaagccacgccggaagaacccccaggaggggctgtacaatgagctgcagaaggataagatggccgaggcctattccgagatcggaatgaagggcgagcggcggcggggcaaggggcacgacgggctgtaccaggggctgagcaccgcaacaaaggacacctatgatgccctgcacatgcaggctctgcccccccgg

SEQ ID NO: 164 - RNA, 1332 nt, synthetic construct
  source 1..1332: mol_type = other RNA
atggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcagagtgaagttcagcagaagcgctgacgcccctgcttaccagcagggacagaaccagctgtttaacgagctgaacctggggagaagagaagagttcgacgtgctggacaagcggagaggcagagatcctgagatgggcggcaagcccagaagaaagaaccctcaagagggcctgtttaatgagctgcagaaagacaagatggccgaggccttcagcgagatcggaatgaagggcgagcgcagaagaggcaagggacacgatggactgttccagggcctgagcacagccaccaaggacacattcgacgccctgcacatgcaggccctgcctccaaga

SEQ ID NO: 165 - RNA, 1509 nt, synthetic construct
  source 1..1509: mol_type = other RNA
atgctgctgctggtgacaagcctgctgctgtgcgagctgccccaccccgcctttctgctgatccccgacatccagatgacccagaccaccagcagcctgagcgccagcctgggcgacagagtgaccatcagctgccgggccagccaggacatcagcaagtacctgaactggtatcagcagaaacccgacggcaccgtgaagctgctgatctaccacacctcccggctgcacagcggcgtgcccagcagattttctggcagcggcagcggcaccgactacagcctgaccatctccaacctggaacaagaagatatcgctacctatttttgtcaacaaggcaacaccctgccctacaccttcggcggaggcaccaaactggaaatcaccggcagcaccagcggctccggcaagcctggatctggcgagggcagcaccaagggcgaagtgaagctgcaagaaagcggccctggcctggtggcccctagccagagcctgtccgtgacctgtaccgtgtccggcgtgtccctgcccgactacggcgtgtcctggatccggcagccccccagaaagggcctggaatggctgggcgtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgtccagcttcgtgcccgtgtttctgcccgccaagcctaccaccacccctgcccctagacctcccaccccagccccaacaatcgccagccagcctctgtccctgcggcccgaagcctgtagaccagctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 166 - RNA, 1467 nt, synthetic construct
  source 1..1467: mol_type = other RNA
atggcattaccagttacagccctcctcctccctctcgctctcctcctccacgccgccaggccagatatccagatgacacagaccaccagcagcctcagcgccagcctcggcgatagagtgacaatctcttgcagagcctcccaggacatcagcaagtacctcaattggtaccagcagaagcccgacggcaccgtgaagctcctgatctaccacaccagcagactgcacagcggcgtgccttctagattctctggaagcggcagcggcaccgactacagcctgaccatcagcaacctggagcaggaggacatcgccacctacttctgccagcagggcaataccctgccctacaccttcggcggcggcacaaaactggaaattacaggtggtggtggttctgggggaggagggtctggtggtggtggtagtgaagtgaaactgcaggaatctggaccaggactggtggccccatctcagtctctgagcgtgacatgtacagtgtctggcgtgtctctgcctgattacggagtgtcttggattagacagcctcctcggaagggcctggagtggctgggagtgatctggggcagcgagaccacctactacaacagcgccctgaagtccaggctgaccatcatcaaggacaacagcaagtcccaggtgttcctgaagatgaacagcctgcagaccgacgacaccgccatctactactgcgccaagcactactactacggcggcagctacgccatggattactggggccagggcaccagcgtgacggtgtcttctacaacaacacctgctcctaggcctcctacaccagctcctacaattgcttctcagccgctgtctctgcggccagaagcttgtaggcctgctgcaggaggagcggtgcatacaagaggactggactttgcctgtgatatctacatctgggcccctctggccggcacctgcggcgtgctgctgctgagcctggtgatcaccctgtactgcaaccacagaaacagaagcaagagaagcaggctgctgcacagcgactacatgaatatgacacctaggagacctggccctacaagaaaacattatcagccttatgcccctcctagagattttgccgcctatagaagccgggtgaagttttctagatctgctgatgccccagcctatcagcagggccagaaccagttatataatgaactgaacctgggccggcgggaggagtacgatgtgctggataagagaagaggcagagatcctgaaatgggaggaaaacctagaagaaagaatccccaggagggcctatataacgaactgcagaaggacaagatggccgaggcctacagcgagatcggcatgaagggcgagagaagaagaggcaagggccacgatggactgtatcaggggctgagcaccgccaccaaggatacctacgatgccctgcacatgcaggccctgcctcctaga

SEQ ID NO: 167 - RNA, 1467 nt, synthetic construct
  source 1..1467: mol_type = other RNA
atgctattattagtcaccagcctcctcctttgcgagctcccccaccccgccttcctcctcatccccgacatccagatgacccagaccaccagcagcctcagcgccagcctcggcgatagagtgacaatctcttgcagagcctcccaggacatcagcaagtacctcaattggtaccagcagaagcccgacggcaccgtgaagctgctgatctaccacaccagcagactgcacagcggcgtgccttctagattctctggaagcggcagcggcaccgactacagcctgaccatcagcaacctggagcaggaggacatcgccacctacttctgccagcagggcaataccttgccctacaccttcggcggcggcacaaagctggaaattacaggtggtggtggtagtggtggtggtggttctggtggtggtggatctgaagtgaaactgcaggaatctggacctggcctggtggccccttctcagtcgctgagcgtgacatgtacagtgagcggcgtgagcctgcctgattatggagtgagctggatcagacagcctccaagaaagggcctggagtggctgggcgtgatctggggctccgagaccacctactacaacagcgccctgaagtccaggctgaccatcatcaaggacaacagcaagagccaggtgttcctgaagatgaacagcctgcagaccgatgacaccgccatctactactgcgccaagcactactactacggcggcagctacgccatggattactggggccagggcacatctgtgacagtgtcttctgctgctgctccagctcctagacctcctacacctgctcctacaattgcttctcagccgctgagcctgagacctgaagcttgtagacctgctgctggaggagctgtgcatacaagaggactggattttgataccgccctggccgccgtgatctgttctgccctggccacagtgctgctggccctgctgatcctgtgcgtgatctactgcaagagacagcccagaagaaagaagctgctgtacatcttcaagcagcccttcatgagacctgtgcagacaacacaggaggaggatggctgttcctgtagatttccagaggaagaggagggcggctgcgagctgagagtgaaattttctagatctgccgatgcccctgcctaccagcagggccagaaccagctatataatgaactgaacctgggcagaagagaggagtacgacgtgctggataagagaagaggcagagatcctgaaatgggaggaaaacctagaagaaagaatccgcaggagggcctatataacgaactgcagaaggacaagatggccgaggcctacagcgagatcggcatgaagggcgagagaagaagaggcaagggccacgatggactgtatcaggggctgagcacagccaccaaggatacctacgatgccctgcacatgcaggccctgcctcctaga

SEQ ID NO: 168 - RNA, 1455 nt, synthetic construct
  source 1..1455: mol_type = other RNA
atggctctacccgttacagcattactactacctcttgccctcctcctccacgccgccagaccagatatccagatgacccagaccacctctagcctcagcgccagcctcggcgatagagtgacaatctcttgcagagcctcccaggacatcagcaagtacctcaattggtaccagcagaagcccgacggcaccgtgaagctcctgatctaccacaccagcaggctgcactctggcgtgccatctagattttctggaagcggcagcggcacagattacagcctgaccatcagcaatctggagcaggaggacatcgccacctacttctgccagcagggcaataccctgccctacaccttcggcggcggcacaaaactggaaattacaggtggtggtggttcagggggaggagggtctggtggtggaggttctgaagtgaaactgcaggaatctggacctggcctggtggccccttctcagtcgctgagcgtgacatgtaccgtgagcggcgtgagcctgcctgattacggggtgagctggatcagacagccccctagaaagggcctggaatggctgggcgtgatctggggcagcgagaccacctactacaacagcgccctgaagtccaggctgaccatcatcaaggacaacagcaagtcccaggtgttcctgaagatgaacagcctgcagaccgacgacaccgccatctactactgcgccaagcactactactacggcggcagctacgccatggattactggggccagggcacctctgtgacagtgagcagcgccgctgccattgaggtgatgtaccctcccccctacctggacaacgagaagtccaacggcaccatcatccacgtgaagggcaagcacctgtgccctagccccctgtttccaggcccatctaagcctttttgggtgctggtggtggtgggcggcgtgctggcctgctacagcctgctggtgaccgtggccttcatcatcttctgggtgagaagcaagaggtcccggctgctgcacagcgactacatgaatatgacacccaggagacctggccctacaagaaaacattatcagccttatgcccctcctagagattttgccgcctatagaagccgggtgaagttttctagaagcgctgatgccccagcctaccagcagggccagaaccagctgtacaacgagctgaacctgggcagaagagaggagtacgacgtgctggataagagaagaggcagagatccggaaatgggaggaaagcctagaagaaagaatcctcaggagggcctgtacaatgagctgcagaaggacaagatggccgaggcctactctgagatcggcatgaagggcgagagaagaagaggcaagggccacgatggcctgtaccagggcctgagcacggccaccaaggatacctacgatgccctgcacatgcaggccctgcctcctaga

SEQ ID NO: 169 - protein, 486 aa, synthetic construct; identical to SEQ ID NO: 58

SEQ ID NO: 170 - protein, 444 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRVKFSRSADAPAYQQGQNQLFNELNLGRREEFDVLDKRRGRDPEMGGKPRRKNPQEGLFNELQKDKMAEAFSEIGMKGERRRGKGHDGLFQGLSTATKDTFDALHMQALPPR

SEQ ID NO: 171 - protein, 503 aa, synthetic construct; identical to SEQ ID NO: 60

SEQ ID NO: 172 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAPAPRPPTPAPTIASQPLSLRPEACRPAAGGAVHTRGLDFDTALAAVICSALATVLLALLILCVIYCKRQPRRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 174 - protein, 485 aa, synthetic construct; identical to SEQ ID NO: 59

SEQ ID NO: 175 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 176 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcactacccgttacagcattactattacctctcgccctcctcctccacgccgccagaccagatatccagatgacacagaccacctctagcctcagcgccagcctcggcgatagagtgacaatctcttgcagagcctcccaggacatcagcaagtacctcaattggtaccagcagaagcccgacggcaccgtgaagctcctgatctaccacaccagcaggctgcacagcggcgtgccatctagattctctggatctggatctggcaccgattacagcctgaccatcagcaatctggagcaggaggacatcgccacctacttctgccagcaggggaataccctgccctacacctttggcggcggcacaaagctggagattacaggtggtggtggttctgggggaggagggtctggtggtggtggtagtgaagtgaaactgcaggaatctggacctggcctggtggccccttctcagtcgctgagcgtgacatgcaccgtgtctggcgtgagcctgccggattacggcgtgagctggattcggcagcctcctcggaagggcctggaatggctgggcgtgatctggggctccgagaccacctactacaacagcgccctgaagtccaggctgaccatcatcaaggacaacagcaagtcccaggtgttcctgaagatgaacagcctgcagaccgatgacacggccatctactactgcgccaagcactactactacggaggctcctacgccatggattactggggccagggaacctccgtgacagtgtcttctacaacaacaccagctcctaggcctcctacacctgctcctacaattgcttctcagcctctgtctctgagaccagaagcttgtagaccagctgctggaggagctgtgcatacaagaggcctggatttcgcctgtgacatttacatctgggctccactggccggcacctgtggcgtgctgctgctgagcctggtgatcaccctgtactgcaagaggggcagaaagaagctgctgtacatcttcaagcagcctttcatgcgccccgtgcagacaacacaggaggaagatggctgcagctgtagatttcctgaagaagaggagggcggctgcgagctgagagtgaaattttctagaagcgccgacgcccctgcctaccagcagggccagaatcagttatataacgaactgaatctgggccggcgggaggagtacgacgtgctggataagagaagaggcagagatcctgaaatgggaggaaaacctagaagaaagaatccgcaggagggcttatataatgaactgcagaaggacaagatggcggaggcctacagcgagatcggcatgaagggcgagagaagaagaggcaagggccacgatggcctgtaccagggcctgagcacggccaccaaggatacctacgatgccctgcacatgcaggccctgcctcctagataatagaaaaacaaaaaacaaaaaccacacaaatggtcgccgaaactactgaaagcat

SEQ ID NO: 177 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattaccagttacagccctccttcttcctctcgccctcttgctccacgccgccaggccagatatccagatgacacagaccaccagcagcctcagcgccagcctcggcgatagagtgacaatctcttgcagagcctcccaggacatcagcaagtacctcaattggtaccagcagaagcccgacggcaccgtgaagttgctgatctaccacaccagcagactgcacagcggagtgccttctagattctctggaagcggctccggcacagactactcgctgaccatcagcaacctggagcaggaggacatcgccacctacttctgccagcagggcaataccctgccctacaccttcggcggcggcacaaaactggagattacaggtggtggtggtagtggtggtgggggaagtggtggtggtggttctgaagtgaaactgcaggaatcagggcctggcctggtggccccttctcagtcgctgagcgtgacctgtaccgtgtcaggcgtgagcctgccagattacggagtgagctggattagacagccccctcggaagggcctggaatggctgggcgtgatctggggcagcgagaccacctactacaacagcgccctgaagtccaggctgaccatcatcaaggacaacagcaagtcccaggtgttcctgaagatgaacagcctgcagaccgacgacaccgccatctactactgcgccaagcactactactacggcggcagctacgccatggattactggggccagggcacctctgtgacggtgtcttctacaacaacacctgctcctaggcctcctacaccagctcctacaattgcttctcagccgctgagtctgcggccagaagcttgtaggcctgctgcaggaggagctgtgcatacaagaggactggactttgcctgtgatatctacatctgggcccctctggccggcacctgcggcgtgctgctgctgagcctggtgatcaccctgtactgcaagcgcggccggaagaagctgctgtacatcttcaagcagcccttcatgaggcccgtgcagacaacacaggaggaggatggctgctcctgtagatttcctgaagaggaggagggcggctgcgagctgagagtgaaattttctagaagcgccgatgcccctgcctaccagcagggccagaaccagttatataacgaactgaacctgggcagaagagaggagtacgacgtgctggataagagaagaggcagagatccagaaatgggaggaaagcctagaagaaagaatccgcaggagggcctatataatgaactgcagaaggacaagatggccgaggcctacagcgagatcggcatgaagggcgagagaagaagaggcaagggccacgatggactgtatcaggggctgagcaccgccaccaaggatacctacgatgccctgcacatgcaggcgctgcctcctagataatagaaaaacaaaaaacaaaaaccacacaaatggtcgccgaaactactgaaagcat

SEQ ID NO: 178 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattaccagttacagccctcctcctccctctcgccctccttctccacgccgccagaccagatatccagatgacccagaccacctctagcctcagcgccagcctcggcgatagagtgacaatctcttgcagagcctcccaggacatcagcaagtacctcaattggtaccagcagaagcccgacggcaccgtgaagctcctgatctaccacaccagcaggctgcactctggcgtgccatctagattttctggatctggcagcggcaccgactacagcctgaccatcagcaacctggagcaggaggacatcgccacctacttctgccagcaggggaataccctgccctacacctttggcggcggcacaaagctggagattacaggtggtggtggttctggtggtggtggtagtggtggtgggggatctgaagtgaaactgcaggaatctggacctggcctggtggccccttctcagtctctgagcgtgacatgcaccgtgtccggcgtgagcctgccggattacggcgtgagctggattcggcagcctcctagaaagggcctggaatggctgggcgtgatctggggcagcgagaccacctactacaacagcgcgctgaagtccaggctgaccatcatcaaggacaacagcaagtcccaggtgttcctgaagatgaacagcctgcagaccgatgacaccgccatctactactgtgcgaagcactactactacggcggctcctacgccatggattactggggccagggcaccagcgtgacggtgtcttctacaacaacacctgctcctagacctcctacaccagctcctacaattgcttctcagcctctgtcgctgagacctgaagcatgtagacctgctgctggaggagctgtgcatacaagaggactggattttgcctgcgacatctacatctgggccccgctggccggcacctgcggcgtgctgctgctgagcctggtgatcaccctgtactgcaagaggggcaggaagaagctgctgtacatcttcaagcagcccttcatgaggcctgtgcagaccacccaggaggaggatggctgcagctgcagatttcctgaagaagaggagggaggctgcgagctgagagtgaaattttccaggagcgccgacgctcctgcctaccagcagggccagaatcagttatataatgaactgaacctgggccggcgggaggagtacgacgtgctggataagagaagaggcagagatcctgaaatgggaggaaagcctagaagaaagaatccgcaggagggcctatataacgaactgcagaaggacaagatggccgaggcctattccgagatcggaatgaagggcgagagaagaagaggcaagggccacgatggcctgtaccagggcctgagcacggccaccaaggatacctacgacgccctgcacatgcaggccctgcctcctagataatagaaaaacaaaaaacaaaaaccacacaaatggtcgccgaaactactgaaagcat

SEQ ID NO: 179 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctacccgttacagctctactgctgccgctggcactcttgctgcacgcagccagacctgacattcagatgacgcagactacgtcgtccctgtcagcgtcccttggggatcgggttactataagttgccgcgccagccaggatataagtaaatacctcaattggtatcagcaaaagccggatggtaccgtgaagctcctcatctaccatacgtcccggctccactctggtgtgccgagcaggttctccgggagtgggagtgggacagactacagcctcacgatcagcaatctagagcaggaggacatcgctacttacttttgccagcaggggaatacccttccctacactttcggtggcggcacgaagttggagataactggaggcggcgggtctggcggcggtgggtcaggcggtgggggctcggaggtgaagctgcaagaaagcggtccagggctggtggccccttcacagtcccttagtgtgacctgcactgtcagcggagtctcactgcccgattacggggtgagctggatcaggcaaccgccgaggaaggggctggaatggttaggtgtcatctgggggtcagagactacttactataacagcgctttgaagtctagactgactatcattaaggataactccaaatcccaagtcttcctcaagatgaacagtttacagacagatgacacagccatctactactgtgccaagcactactactacggaggctcatatgcgatggactattggggccaaggtacgtcggttacagtaagctcgacgaccactcccgcacctcggcctcccacccctgctcctaccatcgccagtcaacccctgtccctgcggcctgaggcctgtcgtcccgcggccgggggtgcggtccatacgcgcggacttgactttgcgtgtgatatctacatttgggcccccttagctggcacatgtggtgttctcctgctctccctggtcatcaccttgtactgcaagcgtggtcgcaagaagttgctatatatatttaagcagcccttcatgcggccagtccagactactcaggaagaagatggctgtagttgccggttccccgaggaagaggaaggcggctgcgagctgcgggttaagttctctcgcagtgccgatgcccctgcgtatcagcaggggcagaatcaactgtataacgagttgaatcttggccgtcgggaagagtacgatgttctcgataaacggcggggtcgagatcccgagatgggagggaagccccggagaaagaacccacaagaggggctttataacgagctgcagaaagacaagatggccgaggcctactcggagatcgggatgaagggagaacggcgccggggtaagggccacgacggcctttaccaagggttaagtaccgcgacaaaagatacttatgacgcactgcacatgcaggcgttgccgccaaggtaatagaaaaacaaaaaacaaaaaccacacaaatggtcgccgaaactactgaaagcat

SEQ ID NO: 180 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccttgccggtcactgcattgctgctgcctctcgcactgctgcttcatgccgcaaggcctgacattcagatgactcagacaaccagctcgctcagcgcatcgctgggggatcgcgtgacgatcagttgccgtgcatcccaggatatttccaagtacctcaactggtaccagcagaagccagatggtacagtaaaattgctgatataccatacatccagattacactcaggcgtaccctcgcgctttagtggctccggaagtggcaccgattactctttgaccatcagtaatctcgagcaagaagacattgcaacgtacttctgccaacaggggaacactctcccatacacctttgggggaggtactaagctcgagattacgggtggcggggggtcgggcggtggcggctctggaggcggagggagcgaggttaaactgcaggagtcaggccctgggcttgtagctcccagtcagtcactgtctgtgacttgtactgtgtcaggtgtttccttgccagactatggggtatcctggatccgccagccaccccgtaaaggtctggagtggctcggggtcatatggggctccgaaactacttattacaactcggcattgaagagcaggctgacaattattaaagataatagtaagtcgcaggtgttccttaaaatgaactccctgcaaactgatgatacggccatatactattgtgccaaacactattattacggcggcagctatgctatggactattggggtcagggcacatctgttaccgttagttcgacgacaacgcccgcaccgcgccctccaaccccagctccgacgattgcatcgcagcccctctcactgcgccccgaggcctgtcggccagccgcaggtggcgctgtgcatacacgggggctggacttcgcatgcgatatttatatctgggctccactggccgggacgtgcggagttcttctcctgtcactggtgatcaccctgtattgtaagcgcgggcggaagaagctgctatatatttttaagcagccctttatgaggcctgtgcagacgacacaggaagaagacgggtgcagttgccgattccctgaggaagaggaggggggctgcgagctgcgcgtgaaattttcgcgctccgctgacgcacctgcgtaccagcaaggccagaatcaactctacaatgagttgaatttggggagacgcgaagagtacgacgtcctcgacaagcgacgagggagggatcctgaaatgggaggaaagcctcgcagaaagaaccctcaggaggggttgtacaatgagttacagaaggacaagatggcggaagcctactccgagatagggatgaagggcgagcggaggcgggggaagggccacgacgggctttatcaaggtctgtccacagccaccaaggatacctatgacgctctgcatatgcaggccctgcccccacgctaatagaaaaacaaaaaacaaaaaccacacaaatggtcgccgaaactactgaaagcat

SEQ ID NO: 181 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcccttccagtgacagcattgttgcttcctttggcactgctgttacatgccgcccgccccgacattcagatgacacagacaacttcctcactgagtgccagtctcggagacagagtgaccatcagctgtcgggcctcccaagatatcagtaaataccttaactggtaccagcagaagcccgatgggacagttaaacttctcatctatcataccagcaggctgcacagcggcgttcctagcagatttagtggtagcgggagcggcactgattacagcctgacaattagcaatctcgagcaggaggatatcgctacctacttttgccagcagggtaacaccctgccctacactttcggggggggaactaaactggagataacagggggtggcgggagcgggggaggtggcagtggggggggcgggagtgaagtgaaactccaggagagcgggccaggtctcgtggccccctctcaatctttgtcagtgacttgcacagtctctggcgtttccctgcccgactatggcgtctcatggattcgacagccccctcgaaaggggttagagtggctcggagtgatatggggctccgaaaccacctattacaattccgctttaaagagcagattgacaatcatcaaggataattccaagtctcaggtgtttttgaaaatgaactcccttcagactgatgacactgcaatctactactgtgccaaacattactattacggggggagttacgccatggactactgggggcagggcacaagtgtgactgtctcctccaccaccactcctgctccccgacctcctacacctgctccaactatcgcctctcagccactgagcctgcgacccgaggcttgcaggcctgcagctggaggcgctgtccataccagaggtctggacttcgcctgcgatatctacatctgggccccactagccggtacctgcggcgtcctcttattgagcctcgtgatcactctttactgcaagagaggccgcaagaagctgttgtacatattcaaacagccttttatgcggcctgtgcagactacacaggaggaagacgggtgctcatgtagattccctgaggaagaggagggtggatgcgagcttcgggtgaagttcagccgaagcgctgacgcccccgcataccaacagggccagaaccaactctacaatgagttgaatttgggccgcagggaagaatacgacgtgctggataaacgaaggggccgagaccccgaaatggggggcaagcctcggcggaagaatccccaggaaggcttgtacaatgagttgcagaaagataaaatggccgaggcctactccgagattggtatgaagggcgaaagaaggcggggaaaagggcacgacgggttgtatcaggggctgagtaccgcaacaaaggacacctatgatgccctacatatgcaggctctacctcccagataatagaaaaacaaaaaacaaaaaccacacaaatggtcgccgaaactactgaaagcat

SEQ ID NO: 182 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctcttcccgtgacagccttgctgctcccattggctctcctactgcacgcagcacgacccgatattcagatgacccagactacctcttccctcagcgcctccctcggggaccgagtgactatctcctgtcgggcatcccaggatattagtaagtacctcaattggtaccagcaaaagcccgacgggacagtcaaactattaatataccacacctctagactacatagtggtgttccttctcggttctccgggagcggctctgggactgactacagtctcaccataagcaacctggagcaggaggacatagccacatacttctgtcagcagggcaacaccctgccctataccttcggagggggcaccaaactagagattactggcggagggggttcaggaggaggcgggagcgggggcggtggaagtgaggtgaagttacaggagtccgggcctgggctcgtcgccccttcccagagcctgtcagttacctgtactgtgagcggagtctcattacccgactacggagtcagctggatccgccagccccccaggaagggactggagtggctgggcgtgatctggggttctgagaccacatattataattctgctctaaaatctaggctgacaatcatcaaagacaacagcaagagccaggtgtttctgaagatgaactcattacaaacagatgacactgctatctactactgcgcaaagcactactattatggtgggagctatgccatggattattgggggcaggggaccagcgtcactgtctccagtacaacaacccccgctccacgacctccaaccccagcacccactattgcttcacagcccctgagtttacggcccgaggcatgtcgccctgccgctggaggggctgttcataccagaggactggacttcgcctgtgatatctacatttgggccccactggccggaacatgtggcgttctcctgctctccctcgtcataacactctattgcaagagaggcaggaagaaacttctctatatctttaaacagccttttatgaggcctgtgcagactacacaggaagaggatggatgttcctgccgattcccagaggaggaggagggcgggtgtgagctacgagtgaagttctcacgctctgcagatgcccccgcttaccagcaggggcagaatcagctctacaacgagctgaatctcggacgccgagaagaatacgacgtgctggacaaacgcaggggtagagaccccgagatggggggaaaacctaggcgcaaaaacccccaggagggcctgtataacgagctgcaaaaggataagatggccgaagcttacagtgagatcggcatgaagggggagcgccgccgcggtaaagggcacgacggtctgtaccagggacttagcacagctaccaaggacacctacgatgctctccacatgcaggcattaccacccaggtaatagaaaaacaaaaaacaaaaaccacacaaatggtcgccgaaactactgaaagcat

SEQ ID NO: 183 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccttgcccgtcactgccctattacttcctctcgccctgttgctccacgccgctcgccccgacatccaaatgacacagaccacctcttccctatccgcatccttgggagatagggttaccatctcctgtagggcatcccaggatataagcaaatatttgaattggtatcagcaaaaacctgatggtacagtgaagctactcatttatcacacatcccggttgcattccggcgtgccctcccgcttcagcggaagcggttccgggactgactattcacttactataagtaatctagagcaagaggacattgcaacttatttctgccagcaaggaaacaccctaccctacacatttggaggaggaacaaaattggaaatcaccgggggcgggggttcaggtggtggtggaagcggtggtggtggatcagaggttaagttgcaagagagcggccccggccttgtggctcccagtcagagtctatcagtgacctgtaccgtgtctggcgttagtctcccagactatggagttagctggatcagacaaccaccacgcaagggactggagtggcttggagttatatggggatctgagaccacatattacaattctgcccttaagtcccgactaacaatcattaaagacaactctaagagccaggttttcctcaaaatgaactctttgcagaccgatgacaccgctatttactattgtgccaagcactactattacggtgggtcatacgctatggattactgggggcagggcacatcagtcactgtgagctctacaaccacacccgctcccaggcctccaacccccgcccccactatcgcctctcaacctctcagcttaaggccagaggcttgtcgacctgctgccggcggagctgttcatacccgcggtttggacttcgcttgcgacatatacatttgggcaccactcgccggcacctgcggtgtgctcctcctttctctcgtgattaccctgtattgcaagcgaggcagaaaaaaattattatatatttttaaacagcccttcatgcggcctgtgcagactacacaagaagaagatgggtgcagctgcaggttccctgaggaggaggaaggcggctgtgaacttcgggtcaaattttcccgcagtgccgatgctcctgcataccaacaaggtcaaaaccagctgtacaatgagttaaatcttggacggagagaagagtatgatgtcttggacaagcggcgaggtcgggatcctgagatgggcggaaagccacggcgcaagaacccacaagaaggtctctacaacgagcttcagaaagataaaatggctgaagcttatagcgaaattgggatgaaaggagaaagacgcaggggaaagggacacgacggactttatcaaggtctatctaccgctaccaaagatacttatgacgccctccacatgcaggcacttcctccacgctaatagaaaaacaaaaaacaaaaaccacacaaatggtcgccgaaactactgaaagcat

SEQ ID NO: 184 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccttgccagtaaccgcacttctgctgccactagcactgctgctgcatgcagcccgaccagatatacaaatgacccagacaacctcgagtttgagtgcctcccttggagatcgcgtaactatcagctgtcgggctagtcaggacatttcaaagtacctcaactggtatcagcagaaacctgatggaactgtgaagctgctgatttaccatacatcacgcttacactccggagtgccctcacgtttctccggatccggttccggaactgattatagtcttaccattagcaaccttgagcaggaagacatagccacctatttctgccaacaaggaaatactttgccttatacctttgggggcggcactaagctggaaatcacaggtggcgggggaagcgggggcgggggaagtggcggtggggggtctgaagtgaagctccaagaatctggaccaggtttagttgcaccgagccaaagcctatccgtgacctgcactgtttccggggtgtctcttccagattacggagtgtcctggatacgccagccgccgcgaaaagggctcgagtggttgggcgtcatatggggcagcgaaacgacctattataacagtgccctgaaaagccggctaacgatcattaaagataactctaagtcacaagtattcctcaagatgaactcccttcagacggatgatacggccatatactactgtgctaagcattattattatggcgggtcatacgcgatggactactggggccaggggacatccgtcaccgtatcctctacgacgacaccggcaccaagacctcctactcctgcacccaccatagccagccagcctttgtcactgagacccgaggcttgtagaccagctgccggtggcgcagtgcacacccgaggcttggactttgcctgtgacatttacatctgggcaccacttgcagggacttgcggggtactcttattgagcctcgtaatcacactgtactgcaagagaggacgcaaaaaattgctatatatttttaaacagccttttatgcgccctgttcagactacgcaggaggaagatggatgttcttgtaggtttcccgaagaagaggagggcgggtgtgagctaagagtaaaattcagtagatctgcggatgcaccggcctaccagcagggtcagaaccagttatacaacgagctaaacttaggacgcagagaagagtacgatgtattagataaacgccgtggtcgcgatcctgaaatgggtggtaagcctcgtcggaagaatcctcaggagggactgtacaacgaactacagaaagataagatggctgaggcttactccgagattggaatgaagggagagcgcagaaggggaaagggacatgacggcctgtaccaaggactgagcactgcaactaaggatacctacgacgctctgcacatgcaggccctgcccccaagataatagaaaaacaaaaaacaaaaaccacacaaatggtcgccgaaactactgaaagcat

SEQ ID NO: 185 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctaccagtcacagcactactcctccctcttgccctccttcttcacgccgccaggccagatatccagatgacacagaccacctcctccctttccgcctctctcggggaccgagtcactatttcttgtcgggcttctcaggatatcagtaagtaccttaattggtaccagcagaagcccgacgggacagtgaagctcctgatctaccacaccagtaggctccactctggtgtcccctccagatttagtgggtcagggagtgggaccgactactccctgaccatctccaatctggagcaggaggatatcgccacctacttctgtcagcaggggaataccctgccatatacatttggagggggcaccaagctggagattactggtggtggtggatcaggaggaggtggaagtggaggaggtggttctgaagtgaaacttcaggaatctggacctggcctggtggctcccagccagtccctgagtgtgacttgcaccgtgtctggcgtgtctctgccagactacggtgtgagctggatccgccagccacccaggaaaggcctggagtggctgggcgtgatctggggcagtgagaccacctactataatagtgctttgaagtccaggctgaccatcatcaaggacaactccaagagtcaggtgttcttgaagatgaacagcctgcagactgatgataccgcaatctactattgcgcaaagcactattattatggggggtcttacgccatggattactgggggcaggggactagtgttactgtgagtagcactactacccctgcccctagacctccaacaccagctcctaccattgcaagtcagcctctgtctctgagaccagaggcttgtagacctgcagctggtggagctgtgcatactcggggactggattttgcatgtgacatttacatctgggctcctctggccggcacctgcggcgtcctcctcctgagtctggtgatcactctgtactgcaagcgcggaaggaagaagctgctgtacatcttcaagcagcccttcatgcgcccagtgcagaccactcaggaggaggacggctgcagctgccggtttccagaggaggaagaaggagggtgtgagctgagggtgaagttttcacgctcagctgacgcccctgcttatcagcagggccagaatcagttatataatgaactgaatctgggcaggagggaggagtacgatgtgctggataaacggagaggcagggatcccgaaatgggaggtaagcctcggcggaagaatcctcaggagggtctatataacgaactccagaaggataagatggccgaggcttactccgagatcgggatgaaaggggagaggcgccggggtaaaggccacgatggcctttaccagggcctctccaccgcaacaaaggatacctatgatgccctgcatatgcaggccctgcctccccggtaatagaaaaacaaaaaacaaaaaccacacaaatggtcgccgaaactactgaaagcat

SEQ ID NO: 186 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcactacccgttaccgctctcttgctccctttggccctcctcctccacgccgccagacccgatatccagatgactcagactacatctagtttgagtgcatctctcggagatcgggtcaccatttcttgtcgggcttctcaggatatcagtaagtaccttaattggtaccagcagaagcccgacgggacagtgaagctcctgatctaccacaccagtaggctccactctggtgtcccctccagatttagtgggtcagggagtgggaccgactactccctgaccatctccaatctggagcaggaggatatcgccacctacttctgccagcaggggaataccctgccatatacatttggagggggcaccaagctggagattactggtggtggtggatcaggaggtggtggtagtggaggaggagggtcagaggtgaaactgcaggagagcggtcctggtctcgttgccccaagtcagtccctgtccgtgacatgtaccgtgtctggcgtgtctctgccagactacggagtgtcatggatcagacagccacccaggaaaggcctggagtggctgggagtgatttggggcagcgagaccacatattataactccgccctgaagtccaggctgaccatcatcaaggacaactccaagagtcaggtgttcttgaagatgaatagcctgcagacagatgacacggccatctactactgtgcgaagcactactactatggcggcagctacgccatggattactgggggcaggggacatctgtgactgtgagttcaacaaccactccagctccacggcctcctactccagctccaaccattgcaagtcagcctctgagtctgagaccagaggcttgtagacctgcagctggtggagctgttcatactcgcgggctggatttcgcctgcgatatctacatctgggctcctctggccggcacctgcggcgtcctcctcctgagcctggtgatcactctgtactgcaagcgcggaaggaagaagctgctgtacatcttcaagcagcccttcatgcgcccagtgcagaccactcaggaggaggacggctgcagctgccggtttccagaggaggaggaaggaggctgtgagctgagagtgaagttttctcgcagcgcagatgcccctgcctaccagcagggccagaatcagttatataatgaactgaatctgggccgcagggaggagtatgatgtgctggataagcggaggggcagggatcccgaaatggggggtaagcctcggcggaagaatcctcaggagggtctatataacgaactccagaaggataagatggccgaggcttactccgagatcgggatgaaaggggagaggcgccggggtaaaggccacgatggcctttaccagggcctctccaccgcaacaaaggatacctatgatgccctgcatatgcaggccctgcccccccggtaatagaaaaacaaaaaacaaaaaccacacaaatggtcgccgaaactactgaaagcat

SEQ ID NO: 187 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctaccagtgacagccctcctcctccctttggccttgttgctccacgcagcaaggccagatattcagatgacacagacaaccagcagcctctccgcctctcttggagacagggtgaccatttcttgtcgggcttctcaggatatcagtaagtaccttaattggtaccagcagaagcccgacgggacagtgaagttgctgatctaccacactagcaggctgcatagcggtgtcccttccagattctcgggatctggaagcggcaccgactacagcctgactatcagcaacctggagcaggaggacatcgccacatatttctgccagcaggggaataccctgccctatacatttggcgggggcacaaagctggagattacaggtggcggaggctctggaggaggaggatcaggcgggggcggatctgaagtgaaacttcaggaatccggccccggcctggtcgctccttcccagagcctgtccgtcacctgtacagtctccggcgtgtccctgccagattatggggtgagttggattcgccagccccctagaaaggggctggaatggttaggcgtgatttgggggtcagagaccacctactacaactctgccctcaagtcacgcctgaccattatcaaggacaatagtaagagccaggtgtttctgaagatgaatagtcttcagaccgatgacaccgctatttactattgtgccaagcactactactatggcggcagctacgccatggattactgggggcaggggactagtgttactgtgagtagcactactacccctgcccctagacctccaactccagctccaaccattgcaagtcagcctctgagtctgagaccagaggcttgtagacctgcagctggtggagctgttcatacaaggggcctggattttgcctgtgacatttacatctgggcccctctggcaggaacatgtggcgtgctcctcctgtctctggtgatcaccctgtactgtaagagaggcaggaagaagctgctgtacatcttcaagcagccattcatgagacctgtgcagacaacacaggaagaggatggctgctcctgcaggttccctgaggaggaggaggggggctgtgaactgcgcgtcaaattctcccggtctgccgatgcccctgcttatcagcaggggcagaatcagttatataatgaactgaatctcggcagacgggaggagtatgacgtgctggataagcggaggggcagggatcccgaaatggggggtaagcctcggcggaagaatcctcaggagggtctatataacgaactccagaaggataagatggccgaggcttactccgagatcgggatgaaaggggagaggcgccggggtaaaggccacgatggcctttaccagggcctctccaccgcaacaaaggatacctatgatgccctgcatatgcaggccctgcccccccggtaatagaaaaacaaaaaacaaaaaccacacaaatggtcgccgaaactactgaaagcat

SEQ ID NO: 188 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattaccagttaccgccctcctcctccccctcgccctcctcctccacgccgcccggcccgacatccagatgacccagaccaccagcagcctcagcgccagcctcggcgaccgggtgaccatctcttgccgggccagccaggacatcagcaagtacctcaattggtaccagcagaagccggacggcaccgtgaagctcctgatctaccacaccagccggctgcactccggggtgcccagccggttcagcggcagcggcagcggcaccgactactccctgaccatcagcaacctggagcaggaggacatcgccacctacttctgccagcaggggaacaccctgccctacaccttcggcggcggcaccaagctggagatcaccggcggcggcggcagcggcggcggcggcagcggcgggggcggcagcgaggtgaagctgcaggagagcggccccggcctggtggcccccagccagagcctgtccgtgacctgcaccgtgtccggcgtgagcctgcccgactacggcgtgtcctggatccgccagccccccaggaagggcctggagtggctgggcgtgatctggggcagcgagaccacctactacaacagcgcgctgaagtccaggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgtccagcaccaccacccccgccccccggccccccacccccgcccccaccatcgccagccagcccctgagcctgcggccggaggcctgcaggcccgccgccggcggcgccgtgcacacccggggcctggacttcgcctgcgacatctacatctgggcccccctggccggcacctgcggcgtgctgctgctgagcctggtgatcaccctgtactgcaagcggggccggaagaagctgctgtacatcttcaagcagcccttcatgcgccccgtgcagaccacccaggaggaggacggctgcagctgccgcttccccgaggaggaggagggcggctgcgagctgcgggtgaagttctcccgcagcgccgacgcccctgcgtaccagcagggccagaaccagctgtacaacgagctgaacctgggccggcgcgaggagtacgacgtgctggacaagcgccggggccgggaccccgagatgggcggcaagccccggcgcaagaacccccaggagggcctgtacaacgagctgcagaaggacaagatggccgaggcctacagcgagatcggcatgaagggcgagcggcggcggggcaagggccacgacggcctgtaccagggcctgtccaccgccaccaaggacacctacgacgccctgcacatgcaggccctgcccccccggtaatagaaaaacaaaaaacaaaaaccacacaaatggtcgccgaaactactgaaagcat

SEQ ID NO: 189 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctgcccgtgaccgccctgctgctccccctggccctgctgctgcacgcagcaaggccagacatccagatgacccagactacgagcagcctgagcgcctccctgggcgacagggtgacaatcagctgccgggccagtcaggacatcagcaagtacctgaactggtaccagcagaagcctgacggcaccgtgaagctgctgatttatcacacctccaggctgcacagcggcgtgccctccagattctccggcagcggctccggcaccgactacagcctgaccatctccaacctggagcaggaggatatcgccacctacttctgccagcaggggaacaccctgccctatacctttgggggcggaaccaagctggagatcaccggcggtggcggatccggcggcggcgggagcggcggcggaggatctgaggtcaagctgcaggagtctggccctgggctggtggccccaagccagagcctgtccgtgacatgtaccgtgtccggcgtgtctctgcctgactacggcgtgtcatggatcaggcagcctcctcgcaaggggctggagtggctgggcgtgatctggggcagcgagaccacctactacaacagtgccctgaagtcccggctgaccatcatcaaggacaacagcaagagccaggtgttcttgaagatgaacagcctccagaccgacgacaccgccatttactactgtgccaagcactattactatggcgggtcctacgccatggattactggggccagggcaccagtgtgaccgtgtccagcaccacgactcctgccccacgcccacccactccagcccctaccatcgccagccagccactgagcctgcggcctgaggcctgcaggccagcggctggcggggcggtgcacacccgcgggctggacttcgcctgcgacatctacatctgggcccccctcgccggcacctgcggcgtcctcctcctgagcctggtgatcactctgtactgcaagaggggccgcaagaagctgctgtatatcttcaagcagcctttcatgcggcccgtgcagaccactcaggaggaggacggctgcagctgccggtttcctgaggaggaggaggggggctgtgagctgcgggtgaagttcagccgctccgccgacgcccccgcctaccagcagggccagaatcagctgtacaacgagctgaatctgggccgcagggaggagtacgacgtgctcgacaagcggcgggggcgggaccccgagatgggcggcaagcccaggcggaaaaacccccaggaggggctgtacaatgagctccagaaggacaaaatggccgaggcttacagcgagatcggcatgaagggggagaggcggaggggcaaggggcatgacggcctgtaccaggggctgtccacagccaccaaggacacctacgacgccctgcacatgcaggccctgcctcctaggtaatagaaaaacaaaaaacaaaaaccacacaaatggtcgccgaaactactgaaagcat

SEQ ID NO: 190 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctgcccgtgaccgccctgctgcttcccctggccctgctgctgcacgccgcaaggccagacatccagatgacccagactaccagttctctgtccgcctctctgggcgacagagtgacgatcagttgccgggccagtcaggacatcagcaagtacctgaactggtaccagcagaagcctgacggcaccgtgaagctgctgatctaccacaccagccggctgcacagcggagtgcccagccggttcagcggctccgggtccggcactgattacagcctgaccatctcaaacctggagcaggaggacatcgccacttacttctgccagcagggcaataccctgccctacaccttcggaggcggcaccaagctggagatcaccggcggcggcggctccggcggcggtggctccggcgggggcggctccgaggtgaagctgcaggagagtggcccaggcctggttgcccccagccagagcctgagcgtgacatgcaccgtgtcaggcgtgagtcttcctgactacggggtgagctggatcaggcagccccctaggaagggcctggagtggctgggggtgatctggggctcagagaccacatactacaactctgccctcaagagcaggctgaccatcatcaaggacaactccaagtcccaggtgttcttgaagatgaacagcctgcagaccgacgataccgccatttactactgtgccaagcactactactatggcgggtcctacgccatggactattgggggcaggggactagtgtgactgtgagctccaccaccaccccagcccccaggccccctactcctgcccctaccatcgccagccagcccctgagcctgcggcctgaggcctgcaggccagccgccgggggcgccgtgcacaccaggggcctggactttgcctgtgacatttacatctgggcccctctggccggcacctgcggcgtgctgctgctgagcctggtgatcaccctgtactgcaagaggggcaggaagaagctgctgtatatcttcaagcagcccttcatgcggccagtgcagaccactcaggaggaggacggctgcagctgccgcttccctgaggaggaggagggcggctgcgagctgcgggtgaagttctcccgcagcgccgatgcccctgcctaccagcagggccagaatcagctgtacaatgagctgaatctgggccgtcgggaggagtacgacgtgctggataagcgcaggggcagagatcctgagatgggcggcaagccccgcaggaagaatcctcaggagggtctgtacaatgagctgcagaaggacaagatggctgaggcttattcagagatcggcatgaagggcgagaggcggcggggcaagggccacgatggcctctatcaggggctgagcacggccaccaaggatacatacgacgccctgcacatgcaggcgctgcctccccggtaatagaaaaacaaaaaacaaaaaccacacaaatggtcgccgaaactactgaaagcat

SEQ ID NO: 191 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctccccgtcaccgccctgctcctccccctggcactcctgctgcacgccgccaggcccgacatccagatgacccagaccaccagcagcctcagtgcctccctgggagatagggtgactatctcctgcagggccagtcaggacatcagcaagtacctgaattggtaccagcagaagcctgacggcactgtgaagctgctgatctaccacacctcccgcctgcacagcggtgtgcccagccggttctccggctctgggtccgggactgactacagcctgaccatctccaacctggagcaggaggacatcgccacctacttctgccagcaggggaacaccctgccatacacctttggtggcgggaccaagctggagatcaccggcggaggcgggagcggcgggggcggctccggcggaggcgggagcgaggtcaagctgcaggagtctggccccggcctggtggcccccagccagagcctgagcgtgacctgcaccgtctccggcgtgagcctgcccgactatggcgtgagctggatcaggcagccacccaggaagggcctggagtggctgggcgtgatctgggggtctgagaccacctactataacagcgccctcaagtcacgcctgaccatcatcaaggacaacagcaagtcccaggtgttcctgaagatgaacagtctgcagacagacgacaccgccatctactactgtgccaagcactactactatggcgggtcttacgccatggactactgggggcaggggacgagtgtgactgtgtccagcacaaccacacccgcccctaggcccccaacacccgcccccactatcgcaagccagcccctgagtctgaggcccgaggcctgccggcccgccgccggcggcgccgtgcacaccaggggcctggacttcgcttgcgatatctacatctgggctcctctggccggaacctgcggcgtgctgctgttgtccttggtgatcactctgtactgcaagaggggccgcaagaagctgctgtacatctttaagcagcctttcatgcggcccgtgcagaccacccaggaggaggacggctgcagctgcaggttcccagaggaggaggagggcgggtgtgagctgagggtgaagttctccaggtctgccgacgcccccgcctaccagcagggccagaatcagctctacaatgagctgaatctgggccgccgggaggagtacgatgtgctcgacaagcggcgggggcgcgacccagagatgggcggcaagcctaggagaaagaaccctcaggaggggctctacaatgagctgcagaaggacaagatggctgaggcgtattcagagatcgggatgaaaggggagaggaggaggggcaaggggcacgacggcctgtatcaggggctgagcaccgccaccaaggacacctatgacgcactgcacatgcaggccctgcccccgcggtaatagaaaaacaaaaaacaaaaaccacacaaatggtcgccgaaactactgaaagcat

SEQ ID NO: 192 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctgcccgtgaccgccctgcttctgcccctggccctgctgctccacgccgccaggccagacatccagatgacccagacaacctccagcctgagcgccagcctgggcgacagagtgaccatcagttgccgggccagccaggacatcagcaagtacctgaattggtaccagcagaagcccgacggcaccgtgaagctgctgatctaccacaccagccgcctgcacagcggcgtgcccagcaggttcagcggcagcggctccggcaccgactacagcctgaccatcagcaacctggagcaggaggacatcgccacctacttctgccagcagggcaataccctgccctacacctttggcggcggcaccaagctggagatcaccggcggcggcggaagcggcggcggcggctccggcgggggcggcagcgaggtgaagctgcaggagagcggcccaggcctggtggcccccagccagagcctgagcgtgacctgcaccgtgagcggcgtgtccctgcctgactacggggtgagctggatcaggcagccccccagaaagggcctggagtggctgggcgtgatctggggcagcgagaccacctactacaattccgccctgaagtcccggctgaccatcatcaaggacaattccaagagccaggtgttcctgaagatgaactccctgcagaccgacgacaccgccatctactactgcgccaagcactactactacggcggctcctacgccatggactactggggccagggcacctccgtgaccgtgagctctaccaccacccccgcccctcggcccccaacacctgcccccaccatcgctagccagcccctgtctctgagacccgaggcttgcagaccagccgccggcggcgccgtgcacaccagaggcctggacttcgcctgcgacatctacatctgggcccccctggccggcacctgcggcgtgctgctgctgagcctggtgatcaccctgtactgcaagaggggaaggaagaagctgctgtacatctttaagcagcctttcatgagacccgtccagaccacacaggaggaggacggctgtagctgcaggttcccagaggaggaggagggcggctgcgagctgcgggtgaagttcagcaggtccgctgacgcccccgcctaccagcagggccagaaccagctgtacaatgagctgaacctgggccggagagaggagtacgacgtgctggacaagaggagaggcagggaccccgagatgggcggcaagcctagaaggaagaacccccaggagggactgtacaacgagctgcagaaggacaagatggccgaggcctactctgagatcggcatgaagggcgagaggagaagaggcaagggccacgatggcctgtaccagggcctgagcaccgccaccaaggacacctacgacgccctgcacatgcaggccctgcccccccggtaatagaaaaacaaaaaacaaaaaccacacaaatggtcgccgaaactactgaaagcat

SEQ ID NO: 193 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattaccagttaccgccctcctcctccccctcgccctcctcctccacgccgcccggccagatatccagatgacccagaccacctcctccctcagcgcctccctcggcgaccgggtgaccatctcttgccgggccagccaggacatcagcaagtacctcaattggtaccagcagaagccggacggcaccgtgaagctcctgatctaccacacctcccgcctgcacagcggggtgcccagcaggttcagcggcagcggcagcggcaccgactactccctgaccatcagcaacctggagcaggaggacatcgccacctacttctgccagcaggggaacaccctgccctacaccttcggcggcggcaccaagctggagatcaccgggggcgggggcagcgggggcgggggcagcggcgggggcggcagcgaggtgaagctgcaggagagcggcccaggcctggtggcccccagccagagcctgtccgtgacctgcaccgtgagcggcgtgtccctgcccgactatggcgtgagctggatccgccagccacccaggaagggcctggagtggctgggcgtgatctggggcagcgagaccacctactacaacagcgccctgaagtccaggctgaccatcatcaaggacaacagcaagagccaggtgttcctgaagatgaacagcctgcagaccgacgacaccgccatctactactgcgccaagcactactactacggcggcagctacgccatggactactggggccagggcacctccgtgaccgtgagcagcaccaccacccccgccccccgcccccccacccccgcccccaccatcgccagccagccgctgagcctgcggccggaggcctgcaggcccgccgccggaggcgccgtgcacacccggggcctggacttcgcctgcgacatctacatctgggcccccctggccggcacctgcggcgtgctgctgctgagcctggtgatcaccctgtactgcaagcggggcaggaagaagctgctgtacatcttcaagcagcccttcatgcggcccgtgcagaccacccaggaggaggatggctgcagctgcaggttccctgaggaggaggagggcggctgcgagctgcgggtgaagttctcccgcagcgccgacgcccccgcctaccagcagggccagaatcagctgtacaacgagctgaatctgggccggcgggaggagtacgacgtgctggacaagcgccggggccgggaccccgagatgggcggcaagccccggcgcaagaacccccaggagggcctgtacaacgagctgcagaaggataagatggccgaggcctacagcgagatcggcatgaagggcgagcggcggagggggaagggccacgacggcctgtatcagggcctgtccaccgccaccaaggacacctacgacgccctgcacatgcaggccctgcccccccggtaatagaaaaacaaaaaacaaaaaccacacaaatggtcgccgaaactactgaaagcat

SEQ ID NO: 194 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattacccgtgacggccctcctactccccttagctctcttgctccacgccgcccgcccggacatccagatgacccagacgacctcgagcttgagtgcgtcactcggggaccgagtgacgatctcttgccgagcgtcgcaggacatctcgaagtacttgaattggtaccagcagaagcccgacgggaccgttaagctattaatctaccacacgagtagattgcatagcggcgttcccagtcggttctctggtagcggttcaggtactgactactcgctgaccatcagcaacctggagcaggaggacatcgccacctacttctgccagcagggtaacacgcttccctacaccttcggtggggggaccaagctggagatcactggtggcggaggtagcggtggcggtggttcgggcgggggcgggagcgaggtgaagctgcaggagagtgggcctggcctcgtcgcgccgtcacagagcctctctgtgacgtgcaccgtgagcggggtcagcctgccggactacggagtgtcctggatccggcagccgccccgcaaaggcctcgagtggctcggggtcatttgggggagcgagacaacgtactacaatagtgcgttgaagtcgcgacttactattatcaaggataatagtaagtcgcaggtctttctgaagatgaactcacttcagactgacgacaccgcaatatactattgcgccaagcactactactatggaggcagttacgcgatggactactgggggcaggggacatccgtgactgtctccagcaccaccacgccggccccgcggccccccacccccgcgccgacgatcgcgtctcagcccctcagcctgaggcctgaggcgtgtcgtccagcggcgggtggggccgtgcacacacggggactagactttgcatgcgacatctacatctgggccccgctggcggggacctgtggagtgttgctgctgagtctagtcatcaccttgtactgcaagcgcggccgtaagaagctcctgtacatcttcaagcagcccttcatgaggcctgttcaaacgacgcaggaggaggacgggtgcagctgccgcttccctgaggaggaggagggcggctgcgagctgagagtgaaattctcacgctcggcggacgccccagcttatcagcagggccagaaccagctgtacaatgagctgaacttaggccgcagggaggagtacgacgtgctcgacaagcgccggggtcgtgacccggagatgggtggcaagccccggcgcaagaatccgcaggaggggctctacaacgagctccagaaggacaagatggcggaggcctactccgagattggcatgaagggggagcgccgtcgtggtaagggccacgacggcctgtaccagggcctgtcgacggccactaaggacacttacgacgccctgcacatgcaggcgttgccgccccggtaatagaaaaacaaaaaacaaaaaccacacaaatggtcgccgaaactactgaaagcat

SEQ ID NO: 195 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattaccagttacagccctgttgctgcccctggccctgctgctgcacgccgcccggccagatatccagatgacgcagaccaccagctccctgtcagcctccctgggggacagggtgaccatcagctgccgggccagccaggacatcagcaagtacctgaactggtaccagcagaagcctgatggcaccgtgaagctgctgatctaccacacctcccgcctgcacagcggggtgcccagcaggttcagcggcagcgggtcaggcaccgactacagcctgaccatctccaacctggagcaggaggacatcgccacctacttctgccagcaggggaacaccctgccctacaccttcggcgggggcaccaagctggagatcactggcggcggggggagcgggggcggggggtctgggggcgggggtagtgaggtgaagctgcaggagagcggaccaggcctggtggcccccagccagagcctgtccgtgacctgcaccgtgtctggcgtgagcctgccagactacggcgtgtcctggatccgccagccacccaggaagggcctggagtggctgggcgtgatctggggcagcgagaccacctactacaacagcgcgctgaagtccaggctgaccatcatcaaggacaacagcaagagccaggtgttcctgaagatgaacagcctgcagaccgacgacaccgccatctactactgtgcgaagcactactactatggcgggtcctacgccatggattactggggccaggggacctcagtgaccgtgagcagcaccactacccccgcccccagaccccccacccccgctcccaccatcgccagccagcctctgagcctgcggccagaggcctgcaggcctgccgccgggggggccgtgcacacccgcgggctggacttcgcctgcgacatctacatctgggcccccctggccggcacctgcggggtgctgctgctgagcctggtgatcaccctgtactgcaagcggggccggaagaagctgctgtacatcttcaagcagcccttcatgcgccccgtgcagaccacccaggaggaggacggctgcagctgccgcttccctgaggaggaggagggcggctgcgagctgcgggtgaagttctcccgcagcgccgacgcccctgcgtaccagcagggccagaatcagctgtacaacgagctgaatctgggcaggcgggaggagtacgacgtgctggacaagcgccggggccgggaccccgagatggggggcaagccccggcgcaagaacccccaggaggggctgtacaacgagctgcagaaggataagatggccgaggcctattccgagatcggaatgaagggcgagcggcggcggggcaaggggcacgacgggctgtaccagggcctgagcaccgccaccaaggacacctacgatgccctgcacatgcaggccctgcccccccggtaatagaaaaacaaaaaacaaaaaccacacaaatggtcgccgaaactactgaaagcat

SEQ ID NO: 196 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctaccagtgacagccctcctcctcccactcgccctcctcttgcacgccgcgcgcccagatatacagatgacccagacgacgtccagcttgagtgcgtcactcggggaccgagtgacgatctcttgccgggcgtcgcaggacatctccaagtacctcaattggtaccagcagaagccagacgggacggtgaagctcctgatctatcacacgtcccgtctgcattctggtgtcccgagccggttctccggctcgggaagtgggactgactacagtctcacgatatccaacctggagcaggaggatatcgcaacgtatttctgccagcagggaaatacgttgccgtacaccttcgggggagggacaaagctggagatcactggtgggggcgggagtggggggggggggagcgggggagggggcagcgaggtcaagctccaggagtctgggccgggcctcgttgccccctcccagtcactctcagtcacttgcaccgtttccggggtgtcgctgccggactacggggtgtcgtggatccggcagccaccccggaaagggctggagtggctgggagtgatctgggggtccgagactacgtactacaatagtgcgttgaagtctcggctcaccataataaaggacaatagtaagtcgcaggtctttctgaagatgaacagtcttcagacggacgacactgcgatttactattgtgccaagcactattattatggtgggtcgtacgccatggattactggggtcaggggacctcagtgaccgtgagcagtacgaccacccccgctccccgcccccccactcccgcccccaccatagcatcccagcctttgtccctccgcccggaggcgtgccgccccgccgcgggcggggcggtgcacacgcgagggctggacttcgcgtgtgacatctatatctgggcgccgctggcggggacgtgcggagttcttctgctctcgcttgtaatcacgctgtattgcaagcgagggcggaagaagctcctgtacatcttcaagcagccgttcatgcggccggtccagacgacccaagaggaggacgggtgctcctgccggttcccggaggaggaggaggggggctgtgagctgcgggtcaagttcagccgttcagctgatgcccctgcgtaccagcaggggcagaatcagctgtacaacgagctgaacttgggccgtcgagaggagtacgacgtcctcgacaagcgtcgaggacgtgatcccgagatggggggtaagccccgccgtaagaatcctcaggaggggctctacaacgagctccagaaggataagatggcggaggcttactccgaaatcgggatgaagggagagcgccgtcgtggcaagggccacgacgggctctaccagggcctctcgacggccaccaaggacacctacgacgccctgcacatgcaggcgttgccgccgcggtaatagaaaaacaaaaaacaaaaaccacacaaatggtcgccgaaactactgaaagcat

SEQ ID NO: 197 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctacccgtgaccgctctgctgctacccctggctctgttactgcacgccgcccgccctgacattcagatgactcagactaccagcagcctgagcgcctcgttgggagaccgcgttaccatctcctgtcgcgctagccaggacatatctaaatatctcaattggtaccaacagaagcctgacggcacagttaaacttctcatttatcatacctcacgcctccattccggagtgcccagtaggttcagtggaagcggcagtggaaccgactatagcctcactattagtaacttggaacaagaggacatagcaacatatttttgccaacaggggaataccttgccatatacattcggtgggggaactaaactcgagatcaccggcggaggtggtagcgggggaggaggatcagggggaggaggtagtgaggtgaagttgcaggagtcaggtccgggactggtcgcgccatcccagtcactttcagtgacatgtacagtgagtggtgtttcactgcccgactacggagtctcctggatccgccagcctccccgaaagggattggagtggcttggggtaatatgggggagcgaaacgacctactataactcagccctcaaaagtaggctgacaattataaaggataacagtaaaagtcaagttttccttaaaatgaacagcctgcagacagatgatacggccatctactactgtgcgaaacattactactacgggggctcctatgccatggattattggggacagggaacctcagtcactgtgtcatctacaaccactccggctccacggcctcctacacccgcacccactattgcctcacagcctctcagcctgagacctgaagcctgcagacccgctgcgggcggcgccgtacacacccgaggtctggattttgcctgtgacatttatatctgggccccgttggctggaacatgcggtgtattattgctgtccctggtgataactttgtactgtaagcggggaaggaagaagctactgtacatcttcaagcagccctttatgcgccctgtgcaaacaactcaagaggaggatggttgttcgtgcaggttcccagaggaggaggaaggcgggtgtgagttgcgtgttaagttttcgagatcagctgacgcccctgcttaccagcaagggcagaaccagctctacaacgagctgaatcttggacgtcgagaagagtacgatgttttggataaaaggcgtgggcgcgatccagagatgggcggtaaaccaaggcgtaagaatcctcaggagggtctctataacgagttgcagaaggataagatggctgaggcttacagcgaaatcggaatgaaaggagagagacggcgaggtaaaggtcacgatggtctctatcagggattgagcacagcgaccaaggacacgtacgacgcccttcacatgcaagcgctgcctccgaggtaatagaaaaacaaaaaacaaaaaccacacaaatggtcgccgaaactactgaaagcat

SEQ ID NO: 198 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgccagtaaccgcgcttctactgccattagccttgctgctccacgcagcccgacccgacattcagatgactcagactactagctctctgagtgcatcgctaggtgatcgtgttacgatttcctgccgggcctcacaggatatttctaaatacctgaactggtaccaacagaagcccgatgggacggtgaagttattgatctaccacaccagtcggttgcactccggcgtcccatccaggttttccgggtcaggcagcggtactgactatagccttaccatcagcaacctggaacaggaagacatagccacctacttctgccagcaaggcaacacactgccctacacctttggtggcggaaccaagctggagattactgggggcgggggttctggtggtggtggttcgggcgggggagggagtgaggtcaagcttcaggaatcgggacctggtctagtggctccatctcagtccctgtccgtgacttgcactgtgtccggcgtgtcgttgccggactacggtgtctcgtggatcaggcaacccccccgcaagggcctcgagtggctaggggtgatttggggctccgaaactacctactacaactccgcgctcaagtctaggctgacgatcatcaaggacaactctaaatcccaagtgttcttaaagatgaacagtctgcaaacagacgataccgcgatatattattgcgctaagcattattattatggtggctcctacgccatggattactggggtcaaggcacttccgttacagtcagttcaacaacaaccccagctccacgtcctccaacaccagccccgaccatcgcctctcagccactgtccctcaggcctgaggcctgccggcctgcggccggaggggcagtgcatacgcgggggttggactttgcttgcgacatttatatctgggcgcccctggccggtacatgtggagtgctccttttgtccctggtgatcaccttgtactgcaagagagggagaaagaagctactctacatctttaagcagccgttcatgcgaccggtccagaccacccaggaagaggacggctgcagctgccggtttcctgaagaggaagaaggcggctgcgaattacgcgtgaagttttcgcgcagcgccgatgctcccgcttatcagcaaggccaaaaccagctctacaatgagttgaacctggggcgacgtgaggagtatgatgttcttgataagcggcggggccgagaccctgagatgggcgggaaaccgcgacgtaagaatcctcaggaggggctgtacaacgagctccagaaagataagatggcggaagcgtatagcgagatcggtatgaagggggagcgccggcgcgggaagggtcacgatggactgtaccagggcctcagtaccgcgacaaaggatacctatgacgcgttgcatatgcaggccctgcccccaaggtaatagaaaaacaaaaaacaaaaaccacacaaatggtcgccgaaactactgaaagcat

SEQ ID NO: 199 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctgcctgtgaccgccctgctgctgccactggccctgctgctgcacgccgcccggccagatatccagatgacacagacaacctccagcctgagcgccagcctgggggacagagtgaccatcagctgccgggcctcccaagacatcagcaaatacctgaactggtaccagcagaaacccgatggcaccgtgaagctgctgatctaccacacttccaggctgcacagtggggtgccctcccggttctccggcagcggcagcggcaccgactacagcctgaccatcagcaacctggagcaggaggacatcgccacctacttttgccagcagggcaacaccctgccctacaccttcggcggcggcaccaagctggaaatcaccgggggcgggggatctgggggcggcggctccggcggcggaggatccgaggtgaagctgcaggagtccggcccaggcctggtggcccccagccaaagtctgtccgtgacctgtaccgtgtccggcgtgtccctgcccgactacggcgtgagctggattcgccagcccccccggaagggcctggagtggctgggcgtgatctggggcagcgagaccacctattacaacagcgccctgaagtccaggctgaccatcattaaagataatagcaagagccaagtgttcctcaagatgaatagcctgcagaccgacgataccgccatctactactgtgccaaacactactactacggcgggagctacgccatggactactggggccaaggcacctccgtgaccgtgagcagcaccaccaccccagcccccaggccccctacccccgctcctacaattgccagccagccactgtccctgcgccctgaggcctgccggcccgccgccggcggcgccgtgcatacccggggcctggacttcgcctgtgacatctacatctgggcccccctggctgggacctgcggcgtgctgctgctgagcctggtgatcacactgtactgcaaaaggggccggaaaaaactgctgtacatcttcaagcagcccttcatgagacccgtgcagaccacccaggaggaggacggatgctcctgccgcttcccagaggaggaggagggcgggtgcgagctgagagtgaagtttagccgcagcgccgatgctcccgcctaccagcagggccagaatcagctgtataacgaactcaacctgggcagaagagaagagtacgatgtgctggataaaagacggggcagggaccccgagatgggcggcaagccaagaagaaagaatccccaggagggcctgtacaacgagctgcagaaggataagatggccgaagcctacagcgagattggcatgaagggagagcggaggcgcggcaagggccacgacggcctctaccagggcctgtccaccgccaccaaggatacctacgacgctctgcacatgcaagctctgcccccccgctaatagaaaaacaaaaaacaaaaaccacacaaatggtcgccgaaactactgaaagcat

SEQ ID NO: 200 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctgcccgtcaccgccctgctgctgcccctggccctgctgctgcacgccgcccggcccgatatccaaatgacacagaccaccagctccctgagcgccagcctgggcgacagagtgaccatctcctgcagggctagccaggacatcagcaagtatctgaactggtaccagcagaagcccgacggcaccgtgaagctcctgatctatcacacctccaggctgcacagcggcgtgcccagcagattctccggcagcggcagcggcaccgactactccctgaccattagcaacctggagcaagaggacatcgccacctacttctgccagcagggcaacacactgccctatacctttggcggcggcaccaagctggagatcaccggcggcggcggcagcggcggcggcggcagcggcggcggcggctccgaggtgaaactgcaggaaagcggccccggcctggtggccccctcccagagcctgagcgtgacctgcaccgtgagcggcgtgagcctgcctgactacggcgtgtcctggattaggcaaccccccaggaagggactggagtggctcggcgtgatttggggaagcgagaccacctactacaactccgccctgaagtccagactcaccatcattaaagataacagcaaatcccaagtgttcctgaagatgaactccctgcaaaccgacgacaccgccatctactactgcgccaagcactactactacggaggcagctacgccatggactattggggccagggcaccagcgtgaccgtgagcagcactacaacccccgcccccagacctcctacccctgcccccaccatcgcctcacagcctctgagcctgagacccgaggcctgcaggcccgccgccggcggcgccgtgcacacccggggactggacttcgcctgcgacatttacatctgggcccccctggcaggcacctgcggcgtgctcctgctgagcctggtgattaccctgtactgcaagagaggaagaaagaaactgctgtacatcttcaagcagcccttcatgaggcccgtgcagaccacccaggaagaggacggctgctcctgtaggttccccgaggaggaggagggcggctgcgagctcagagtgaagtttagcaggagcgccgacgcccccgcttaccagcaaggccagaaccagctgtacaatgaactgaacctgggcagacgggaggaatacgacgtgctggacaaaaggagaggaagagaccccgagatgggcggaaagccccggcgcaagaacccacaggagggactgtacaacgagctgcaaaaagacaagatggccgaagcttacagcgagatcggaatgaagggcgagaggagacgcggcaagggacacgatggcctctaccagggcctgagcaccgccaccaaagacacctatgatgccctccacatgcaagccctgccccctaggtaatagaaaaacaaaaaacaaaaaccacacaaatggtcgccgaaactactgaaagcat

SEQ ID NO: 201 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatagaaaaacaaaaaacaaaaaccacacaaatggtcgccgaaactactgaaagcat

SEQ ID NO: 202 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcacttcccgttaccgccctcctccttccactcgccctcctcctccacgccgcccgcccagatatacagatgacacagaccacctccagcttgagtgcctccctcggggacagggtgaccatctcttgtcgggcttctcaggacatcagcaagtacctcaattggtaccagcagaagcccgacgggaccgtgaagctcctgatctaccacaccagcaggctgcacagcggcgtgccctccaggtttagcgggtcagggagtgggaccgactactccctgaccatctcaaacctggagcaggaggacatcgccacctacttttgccagcagggcaacaccctgccctacaccttcgggggaggcactaagctggagatcactggcggtggtgggagcgggggtggggggtctgggggcgggggtagcgaggtgaagctgcaggagtccggacccggcctggtcgcccccagccagtccctgtccgtgacatgcaccgtgtctggcgtgagcctgccagactacggcgtgtcatggatcaggcagcccccccggaaggggctggagtggctgggggtgatctgggggtccgagactacctactacaacagcgcgctgaagtccaggctgaccatcatcaaggacaactccaagagtcaggtgttcttgaagatgaacagcctgcagaccgatgacaccgccatatactactgtgcgaagcactactactatggcgggtcatacgccatggattactggggccaggggacctcagtgaccgtgagcagcaccaccacccccgcccccagaccccccacccccgctcccaccatcgccagccagcccctgagcctgcgccccgaggcctgccggcccgcggccggcggcgccgtgcacactcgggggctggacttcgcctgtgacatctatatctgggcccctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggttcccagaggaggaggagggcgggtgcgagctgcgggtgaagttctcccgcagcgccgatgcccctgcgtaccagcaggggcagaatcagctctacaacgagctgaacctgggccgccgcgaggagtacgacgtgctcgacaagcggcggggccgggatcccgagatggggggtaagcctcggcggaagaatcctcaggaggggctctacaacgagctccagaaggataagatggccgaggcttactccgagatcgggatgaagggggagcggcggcggggcaaggggcacgacggcctgtatcagggcctgagtaccgccaccaaggatacttacgacgccctgcacatgcaggccctgcccccccggtaatagaaaaacaaaaaacaaaaaccacacaaatggtcgccgaaactactgaaagcat

SEQ ID NO: 203 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccctaccagtcaccgccctcctcctccccttggccctcctcctccacgccgcccggccagatattcagatgacacagaccacctccagccttagtgcctccctcggagatagggtgaccatttcttgtcgggcttctcaggacatcagcaagtacctcaattggtaccagcagaagcccgacgggacagtgaagttgctgatctaccacactagcaggctgcatagtggcgtgccctcccgcttctctggaagcgggagcggcaccgactacagcctgactatcagcaacctggagcaggaggacatcgccacctacttctgccagcaggggaataccctgccctataccttcgggggaggcactaagctggagattactggcggaggcggcagtggaggaggagggtcagggggaggaggatctgaagtgaaacttcaggagagtgggcccggccttgttgctccttctcagagcctgtccgtgacatgtaccgtgtctggcgtgtctctgccagactacggcgtgtcatggatcaggcagcctcctaggaaggggctggagtggctgggcgtgatctggggtagtgagactacttactacaatagtgccctgaagtcccggctgactattatcaaggataatagcaagtctcaggtgttcctgaagatgaacagcctgcagacagatgacacggccatctactactgtgcgaagcactactactatggcggcagctacgccatggattactggggccagggcactagtgtgacagtgagtagtaccactaccccagctccacgcccacctactccagcccctacaattgcctcccagcctctgtctctgcgccctgaagcttgtcgccctgctgctggtggagctgtgcacaccagagggctggacttcgcctgcgacatctatatctgggctcctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggtttccagaggaggaagagggtggatgtgagctgcgggtgaagttcagccgctctgctgatgcaccagcctaccagcagggccagaatcagctctacaacgagctgaatctgggccgcagggaggagtacgacgtgctcgacaagcgcaggggcagagatccagagatgggaggcaaacctaggaggaagaatccccaggaggggctctacaacgagctgcagaaggacaagatggccgaggcctactctgagatcggaatgaagggcgagaggaggcggggaaaaggccacgatggcctgtatcaggggctgtccaccgccaccaaggacacctatgatgccctgcacatgcaggccctgcctcctcggtaatagaaaaacaaaaaacaaaaaccacacaaatggtcgccgaaactactgaaagcat

SEQ ID NO: 204 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctcccagtgaccgccctcctccttccccttgccctcctcttgcacgccgctagacccgacatccagatgactcagactacctctagtttgagtgcatccttgggtgatcgggtcaccatctcttgtcgggcttctcaggatatcagtaagtaccttaattggtatcagcagaagcccgacgggaccgtgaagctcttgatctaccatacatcaaggcttcacagcggcgtgccctccaggtttagcgggtcagggagtgggaccgactactccctgaccatctcaaacctggagcaggaggacatcgctacttacttctgccagcagggtaacaccctgccctacacctttgggggagggacaaagctggagatcactggtgggggcgggagtggggggggggggagcgggggtgggggcagcgaggtcaagctccaggagtctgggcctggcctcgttgccccatcccagagtctgtcagtgacatgcactgtgagtggcgtgtcactgccagactatggggtgtcctggatccgccagccacccaggaagggcctggagtggctgggcgtgatctgggggagtgagaccacctactataatagtgctttgaagtctagacttactattatcaaggataatagtaagtctcaggtctttctgaagatgaacagtcttcagacagacgacaccgcaatatactattgcgcaaagcactattattatggggggtcttacgccatggactactggggtcagggaacgtccgtgaccgtcagtagtaccaccacccccgctccccgcccccccactcccgcccccaccattgcatcccagcctttgtccctccgcccagaggcctgtaggccagccgccgggggtgcggtgcacacccgggggctggacttcgcatgtgacatctacatatgggctcctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggttcccagaggaggaggagggggggtgtgagctgagggtgaagttttcacgctcagctgacgcccccgcctaccagcagggccagaatcagctctacaacgagctgaatctgggccggcgggaggagtacgatgtcctcgacaagaggagggggagggatcccgagatggggggtaagcctcggcggaagaatcctcaggaggggctctacaacgagctccagaaggataagatggccgaggcttactccgagatcgggatgaagggggagcggcggagggggaagggccacgatggactgtatcaggggttgtccactgccacaaaggacacctatgatgcactccatatgcaggccctgccccctcggtaatagaaaaacaaaaaacaaaaaccacacaaatggtcgccgaaactactgaaagcat

SEQ ID NO: 205 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctcccagttacagccctcttgttgcctttggccttgctcttgcacgccgcaaggcccgatatccagatgacccagaccaccagttccctaagcgccagcttgggggaccgggtgaccatctcttgtcgggccagtcaggatatcagcaagtacctcaattggtaccagcagaagccggacggcaccgtgaagttgctgatctatcacaccagtaggctgcactccggcgtgccctcccggttctccgggagcggcagcgggacagactacagtctgaccatcagcaacctggagcaggaggacattgccacctacttctgccagcaggggaacaccctgccctatacatttggcgggggcaccaagctggagattacaggcgggggaggctctggcggcgggggcagcggggggggcggctctgaggtgaagcttcaggagtccggccccggcctcgttgcccccagccagagcctctccgtgacctgtaccgtctccggcgtgtccctgccagattatggggtgtcctggatccgccagccaccccggaaggggctggagtggctgggcgtgatctggggctcagagacaacctactacaactctgccctgaagagtagacttactattatcaaggataatagtaagtctcaggtgtttctgaagatgaatagcctgcagacagatgacacggccatctactactgcgctaagcactactactatggcggcagctatgccatggactactggggccaggggactagcgtgacagtgtcctccaccaccacccctgctcccaggccccccacgcctgcccccaccatcgcgagccagccactgtccctccggccagaggcctgccggcctgctgccggaggggcagtgcacacaagagggctggatttcgcctgtgacatttacatctgggctcctctggccggcacctgcggcgtcctcctcctgagcctggtgatcaccctctactgcaagaggggcaggaagaagctgctgtacatcttcaagcagcctttcatgaggcccgtgcagaccacccaggaggaggacggctgcagctgccggttcccagaggaggaggagggcggctgcgagctgcgggtgaagttctcccgcagcgcagacgcccccgcctaccagcagggccagaatcagctgtacaacgagctgaatctgggccggcgggaggagtacgacgtgctggacaagcgccggggccgggaccccgagatggggggcaagccccggcgcaagaatcctcaggaggggctgtacaacgagctccagaaggataagatggcggaggcctacagcgagatcgggatgaagggcgagcgcaggcggggcaaggggcatgacgggctgtaccagggcctgtcaactgccaccaaggacacctacgacgccctgcacatgcaggccctgcctccccggtaatagaaaaacaaaaaacaaaaaccacacaaatggtcgccgaaactactgaaagcat

SEQ ID NO: 206 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccttaccagttacggcgctattgctcccgttggcgctattgctccacgccgcgcgcccggacatacagatgacgcagacgacgtcttcgttatcggcgagtttgggcgaccgggttacgatatcgtgcagggcttcgcaggacataagtaagtaccttaattggtaccagcagaagcccgacggtacggtaaaactactcatctatcatacgtcgcgtttgcatagtggagtaccctcccgtttttcggggagtgggtcgggtaccgactattcgctaacgatatcgaacttggagcaagaagatatcgcgacgtacttttgccagcaagggaatacgttaccgtacacgtttggtggaggtacgaaactagagatcacgggtggtggtggttcgggtggtggtggttcgggtgggggcggctcggaagtgaaacttcaagagtcgggtcccggtctagtggcgccgagccaatcgctatcggtaacgtgtacggtttcgggtgtatcgctaccggattacggtgtatcgtggatacgccaaccgccgcgtaagggcctagaatggctaggggttatatggggtagtgagactacctattataactcggcgttgaaatcgcgtctaacgataataaaagataattcaaaatcgcaggtttttctaaaaatgaattcgctacaaacggatgatacggcgatatattattgtgcgaagcactattattatggtggttcctatgcgatggattattggggtcaaggtacgtcggtaaccgtgtcgtcgacgacgacgccggcgccgcgcccgccgacgccggcgccgacgatagcgtcgcaaccgctatcgttgcggccggaggcgtgccgcccggcggcgggcggggcggtgcatacgcgcggcctcgattttgcgtgtgacatttacatatgggcgccgctagcgggcacgtgtggtgtcctactactatcgttggtgatcaccctatattgtaagcgtggcaggaagaagcttctttatatatttaaacagccgtttatgcggccggtacaaacgacgcaagaagaagatggttgttcgtgccgcttcccggaagaagaagaaggtggctgtgagctacgcgtaaagttttcgcgttcggcggatgctccggcgtatcaacaaggtcaaaatcagttgtataacgaactgaatctagggcgtcgtgaagaatacgacgtcctagataaacgtcgtggtcgggacccggaaatgggtggtaagccgcgtcgtaaaaacccgcaagaggggttatataatgaactccaaaaggataaaatggcggaggcgtattcggagatcgggatgaaaggcgagcgtcgccgtggtaagggccacgacggcctttatcaaggtctctcgacggcgacgaaggatacgtatgatgcgttgcatatgcaagcgttgccgccgcgttaatagaaaaacaaaaaacaaaaaccacacaaatggtcgccgaaactactgaaagcat

SEQ ID NO: 207 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcactaccggtaacggcgctactactaccgctagcgttactattgcatgcagcacgtccggatatacaaatgacgcaaacaacttcgagtttatcggcatctttaggagaccgtgtaaccatatcgtgtcgagcgtctcaagacatatctaagtatttaaattggtatcaacaaaaacctgacggcacggtaaaattactaatataccatacgtcgcgactccattcgggtgtaccatcgcgtttttcgggatcgggttcgggtactgattactcgcttactatatcgaatttagaacaagaagatatcgcgacttatttttgccaacaagggaacacgctaccttatacctttggtggagggaccaaattggaaataacgggggggggcgggagtggaggaggaggttcagggggggggggttcagaagttaaattacaagaatcaggaccaggattagtagcgccgtcgcaatcgttatcagttacgtgtacggtctcgggtgtaagtctaccggattacggagtatcgtggattaggcaacccccgcgcaaggggcttgaatggcttggcgttatctggggctcggaaactacgtattataactcggctctaaaatcacgtttgacgataataaaagacaattcgaaatcgcaagtattcttaaaaatgaacagcctacaaacggacgacacggcgatatactattgtgcgaaacattattattatggtggtagttatgcgatggattactggggccaaggaacaagcgttaccgtatcgtccactacgacgcctgcgccgcgaccgcccacgccggcgcctaccatagcttcacaaccattaagcctccgtccagaggcgtgccgtcctgcggcgggtggtgctgtacatacgaggggtctagactttgcgtgtgatatttatatatgggctccattagcggggacatgcggggttctactactatcgctagtaataactctatactgcaaacgtggacgtaaaaagttactatatatatttaaacagccgttcatgagaccggtacaaactacgcaagaggaagatgggtgctcctgtagatttccggaggaagaagaaggtggttgcgaattaagagttaaattcagtcgaagtgcggacgcgccagcgtatcaacaagggcaaaatcaactatacaatgaactaaatcttggtcgaagagaagaatatgacgtattagacaaacggcgtggacgagaccctgagatgggtggaaaaccgcgaagaaaaaatccacaagaaggtttatacaatgaattacaaaaagataaaatggcggaggcatatagtgaaataggtatgaaaggagaacgaagacgtggtaaagggcatgacggattatatcaaggattgtcgacggcaacgaaagatacttacgacgcgttacatatgcaggccctaccgccgcgataatagaaaaacaaaaaacaaaaaccacacaaatggtcgccgaaactactgaaagcat

SEQ ID NO: 208 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggccttaccagtaacagccttattacttccacttgccctccttctacatgccgctagaccagatatccaaatgacacaaacgacctcaagcctttccgcgtctctcggtgacagggtaactattagttgtcgggcctcccaagatatttcaaaatacttaaattggtatcaacagaagccagatggtaccgtgaagctcctcatctaccacactagccgtcttcactctggagtaccaagcaggttttccggctccggaagtggtactgactacagtttgactataagcaacctagagcaagaggatattgccacatacttctgtcaacaaggaaacactctgccatatacattcggtggtggtacaaaactggaaataactggagggggaggttcaggcggcggcggatctggggggggtggcagtgaggtcaaattgcaggagtctggacccggtctggttgctccatcccaatcactttcagtgacctgcactgtatccggagtttctctgccagattatggagtgagttggatccgccagcctccacgcaagggccttgaatggctcggggttatatggggtagtgaaactacatactataatagcgctctgaaatctcgactcacaatcattaaggataatagcaaaagccaggtgtttttgaagatgaatagcctgcaaaccgatgataccgccatatattactgtgccaaacattactattacggaggctcctacgctatggattattggggacaggggacaagcgtgaccgttagctctactactacccccgcccctcgccctccaactccagctcccaccattgccagtcaaccactatccttaagacccgaggcatgcagacccgccgcagggggcgccgtacatacccgaggtttggattttgcctgcgacatctatatctgggccccccttgctgggacatgtggagtgttgctactctcgttggttatcactctgtattgcaagagaggaagaaagaaactattatatatttttaagcagccattcatgagaccggtgcagactacccaagaagaagacggatgttcatgtaggttccctgaagaagaggaaggaggctgcgaactgcgagtgaaattttccagatctgccgatgcaccagcttatcaacaaggacaaaaccagctatataatgaacttaatttagggagacgagaggagtacgacgtcctggacaaacgccgcggaagagaccctgagatgggaggtaagcccagaagaaaaaacccacaagaaggactgtacaatgaactccaaaaagacaaaatggctgaagcgtattccgaaattggcatgaaaggagaacggagaagaggtaagggtcacgacggcttatatcagggactctctacagcaaccaaagacacatatgatgcactacacatgcaggcactaccacccaggtaatagaaaaacaaaaaacaaaaaccacacaaatggtcgccgaaactactgaaagcat

SEQ ID NO: 209 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctaccagtcacagcactactattgccgttggctctattgcttcacgcggcgcgaccggatatacagatgacccaaacgacgtcgagtctctcggcgtcgttgggtgatcgtgtaacgatatcgtgtcgtgcgtcgcaagatatctcgaaatatctaaattggtatcaacaaaaaccggacgggacggtgaaattattaatatatcacacgtcccgtctacattctggtgttccgagtcgatttagtgggagtggttcgggtacggactactccctaactatatcgaacttggaacaagaggatatagcgacgtacttctgccaacaaggtaatacgctaccgtatacctttggcggaggtacgaagctagaaataactggtgggggtgggagtggtggtggtggttcgggtggtggtggttctgaagtgaaacttcaagaatcgggaccggggctggtagccccgtcccaatcgttatcggtaacgtgcaccgtatcgggtgtatcgttaccggattatggtgtatcgtggatacgccaaccgccccgtaaaggcctagaatggctaggtgttatatggggttcggaaacgacgtactacaatagtgcgttgaaatcgcgacttactattatcaaggataatagtaagtcgcaagtatttttaaagatgaattcgttacagacggacgataccgcgatatactattgcgcgaagcactattattatggtggttcgtatgcgatggattattggggtcaaggtacgtctgtaacggtttcatctactactactccggcaccacgaccaccaactcctgctcctactatagcgtcgcaaccgttatcgttgcgacctgaagcatgtagaccagcggcaggtggtgctgttcatacgcgaggtctagacttcgcgtgtgatatatatatatgggccccgcttgcgggtacgtgtggtgtactattactatcgttagtaataacgctatactgcaagcggggccgaaaaaaattattatatatatttaaacaaccgtttatgcggccggtacaaacgacgcaagaggaagatgggtgttcttgtcgttttcctgaagaggaagaaggtggatgcgaactacgtgtaaaattttcgcgtagtgcggatgcgccggcctatcaacaaggccaaaatcaactctataacgagttgaatctaggacgtcgtgaagagtacgacgtcctagataaacgacgtggtcgtgacccggaaatgggtggcaaaccgcgtcgtaaaaacccgcaagagggtttatataatgagctacaaaaagataaaatggctgaagcgtactcggaaattggtatgaaaggtgaacgtcgtcgtggtaaaggccatgacggtttatatcaaggtctgagtacggcgacgaaggacacctatgatgctctacatatgcaagcgttaccgccgcgttaatagaaaaacaaaaaacaaaaaccacacaaatggtcgccgaaactactgaaagcat

SEQ ID NO: 210 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctacccgtcacggcgttattacttccgctcgcgttgctattacacgcagcgcgaccggatatacaaatgacgcaaacgacgtcgtcgttatcggcgtcgctaggagatcgtgtgacgatctcttgccgagcgtcgcaagatatctcgaaatatctaaattggtatcaacaaaaaccggacggtacggttaaattgttgatatatcatacgtcgaggctacattctggtgttccgagtcgatttagtgggagtggttcgggtacggactactccctaactatatcgaacttggaacaagaggatatagccacgtatttttgccaacagggcaataccctcccctacacgtttgggggagggacgaaactagaaattacaggtggtggtggttctgggggtggtggatcaggtggtggtggttctgaagtgaaacttcaagaatcaggaccgggtctagtcgcccccagtcaatcgctatcggtaacctgtacggtttctggtgtcagcttaccggattatggtgtaagctggatacgtcaaccgccccgtaaaggcctagaatggctaggtgttatatggggttcggagacgacgtactacaatagtgcgttaaaatcgcgacttactattatcaaagataatagtaagtcgcaggtttttttaaagatgaactctttacaaacggatgatacggcgatatactattgcgcgaagcactattattatggtggttcgtatgcgatggattactgggggcagggtacgtcggtaactgtgagttcgactacgactcctgctcctagaccgccgactccggcgccaactatagcatcgcagcctctatcgcttcgtccggaagcgtgtagaccagctgcaggtggtgctgtacatacgcgaggtctagacttcgcgtgtgatatatatatatgggcgcccctagcgggtacgtgtggggtgctattactatcgttagtaataacgctatattgcaaacgtggtaggaaaaagctactatatatatttaaacaaccttttatgcgtccggtacaaacgacgcaagaagaggatggttgtagttgtcgatttccggaagaagaagagggaggttgcgaactacgagtaaaatttagtcgatcggcggatgcgccggcgtatcaacaaggtcaaaatcaattatataatgaattaaatctaggacgtcgtgaagaatatgacgtcctagataaacgacgtggtcgtgacccagaaatgggtggtaaaccgcggcgtaaaaacccgcaagaaggtttatataatgagcttcaaaaagataaaatggcggaagcttattcggaaataggtatgaaaggtgaacgccgtcgtggtaaagggcatgacggtctttaccaaggtttgtcgacggcgacgaaagatacgtatgatgcgttacacatgcaagcgttaccgccgcgttaatagaaaaacaaaaaacaaaaaccacacaaatggtcgccgaaactactgaaagcat

SEQ ID NO: 211 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattacccgttaccgccctcctcttgcccctcgccctcttgctccacgccgcccggccagatatccagatgacccagaccacctcctccctcagcgcctccctcggcgaccgggtgaccatctcttgccgggcctcacaggacatcagcaagtacctcaattggtaccagcagaagcccgacggcaccgtgaagcttctgatctaccacacctcccggctgcacagcggggtgcccagcaggttcagcggcagcgggtcaggcaccgactacagcctgaccatctccaacctggagcaggaggacatcgccacctacttctgccagcaggggaacaccctgccctacaccttcggcgggggcaccaagctggagatcactggcggcggggggagcgggggcggggggtctgggggcgggggtagtgaggtgaagctgcaggagagcggaccaggcctggtggcccccagccagagcctgtccgtgacctgcaccgtgtctggcgtgagcctgccagactacggcgtgtcctggatccgccagccacccaggaagggcctggagtggctgggcgtgatctggggcagcgagaccacctactacaacagcgcgctgaagtccaggctgaccatcatcaaggacaacagcaagagccaggtgttcctgaagatgaacagcctgcagaccgacgacaccgccatctactactgtgcgaagcactactactatggcgggtcctacgccatggattactggggccaggggacctcagtgaccgtgagcagcaccactacccccgcccccagaccccccacccccgctcccaccatcgccagccagcctctgagcctgcggccagaggcctgcaggcctgccgccgggggggccgtgcacacccgcgggctggacttcgcctgcgacatctacatctgggcccccctggccggcacctgcggggtgctgctgctgagcctggtgatcaccctgtactgcaagcgcgggaggaagaagctgctgtacatcttcaagcagcccttcatgaggcccgtgcagaccacccaggaggaggatggctgcagctgcaggttccctgaggaggaggagggcggctgcgagctgcgggtgaagttctcccgcagcgcagacgcccccgcctaccagcagggccagaatcagctgtacaacgagctgaatctgggccggcgggaggagtacgacgtgctggacaagcgccggggccgggaccccgagatggggggcaagccccggcgcaagaatcctcaggagggcctgtacaacgagctgcagaaggataagatggccgaggcctacagcgagatcgggatgaagggcgagaggaggcgcggcaaggggcacgacggcctgtatcagggcctgtccaccgccaccaaggacacctacgacgccctgcacatgcaggccctgcccccccggtaatagaaaaacaaaaaacaaaaaccacacaaatggtcgccgaaactactgaaagcat

SEQ ID NO: 212 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctaccagttacagccctcttgctccctttggccttgttgctccacgcagcaaggccagacatccagatgactcagactacctctagtttgagtgcatccttgggagaccgggtgactatcagttgccgggcctcccaggacatcagcaagtacctcaattggtaccagcagaagccggacggcaccgtgaagttgctgatctaccacaccagcaggctgcacagcggcgtgccctcccggttctccgggagcgggtcagggaccgactactccctgaccatcagcaacctggagcaggaggacatcgctacttacttctgccagcaggggaacaccctgccctacacctttggcggagggacaaagctggagatcactggtgggggcgggagtggggggggggggagcgggggtggggggtctgaggtgaagctgcaggagtccggccccgggctggtcgccccctcacagagcctgtcggtgacatgcactgtgagcggggtgagcctgcccgactacggggtgagctggatccgccagcccccccggaagggcctggagtggctcggagtgatctggggctccgagaccacttactacaacagcgccctgaagtccaggttgactattatcaaggataatagcaagtctcaggtgttcctgaagatgaacagcctgcagaccgatgacaccgccatatactactgtgcgaagcactactactatggcgggtcatacgccatggactactggggccagggcacctcagtgaccgtcagctccaccaccacccccgctccccgcccccccactcccgcccccaccatcgcatcccagcccctgtccctccggccagaggcctgccggcccgccgcgggcggcgccgtgcacacccgcgggctggacttcgcctgcgacatctacatctgggctcctctggccggcacctgcggcgtcctcctcctgagcctggtgatcactctgtactgcaagcgcggaaggaagaagctgctgtacatcttcaagcagcccttcatgcgcccagtgcagaccacccaggaggaggacggctgcagctgccggttcccagaggaggaggagggcgggtgcgagctgcgggtgaagttctcccgcagcgccgacgcacccgcctaccagcagggccagaatcagctctacaacgagctgaatctgggccggcgggaggagtacgatgtgctggacaagcggaggggcagggatcccgagatggggggtaagcctcggcggaagaatcctcaggaggggctctacaacgagctccagaaggataagatggccgaggcttactccgagatcgggatgaagggggagaggcgccggggtaagggccacgatggcctttaccagggcctctccaccgcaacaaaggacacctatgatgccctgcacatgcaggccctgcccccccggtaatagaaaaacaaaaaacaaaaaccacacaaatggtcgccgaaactactgaaagcat

SEQ ID NO: 213 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctttacccgttacagccctgttgctgccccttgccctgctgctgcacgccgcccggccagatatccagatgacgcagaccaccagctcattgtcagcctccctgggggatcgggtgaccatcagctgccgggccagtcaggacatcagcaagtacctgaactggtaccagcagaagcctgatggcaccgtgaagctgctgatctaccacaccagcaggcttcactccggcgtgccttcccgcttctctgggtccggctccggcacagactacagtctgaccatctccaacctggagcaggaggacatcgctacttacttctgccagcaggggaacaccctgccctacaccttcggcgggggcaccaagctggagatcactggcggtggtgggagcgggggtggggggtctgggggcgggggtagcgaggtgaagctgcaggagtccggacccggcctggtcgcccccagccagtccctgtccgtgacatgcaccgtgtctggcgtgagcctgccagactacggcgtgtcatggatcaggcagcccccccggaaggggctggagtggctgggggtgatctgggggtccgagactacctactacaacagcgcgctgaagtccaggctgaccatcatcaaggacaactccaagagtcaggtgttcttgaagatgaacagcctgcagaccgatgacaccgccatatactactgtgcgaagcactactactatggcgggtcatacgccatggattactggggccaggggacctcagtgaccgtgagcagcaccaccacccccgcccccagaccccccacccccgctcccaccatcgccagccagcccctgagcctgcgccccgaggcctgccggcccgcggccggcggcgccgtgcacactcgggggctggacttcgcctgcgatatctatatctgggcccctctggccggcacctgcggcgtcctcctcctgagtctggtgatcaccctgtactgcaagcgcggaaggaagaagctgctgtacatcttcaagcagcccttcatgcgcccagtgcagaccactcaggaggaggacggctgcagctgccggttcccagaggaggaggaggggggctgcgagctgcgggtgaagttctcccgcagcgcagacgcccccgcctaccagcagggccagaatcagctctacaacgagctgaatctgggccggcgggaggagtacgatgtcctggacaagcgccggggccgggacccagagatgggcgggaagccacgccggaagaacccccaggaggggctgtacaatgagctgcagaaggataagatggccgaggcctattccgagatcggaatgaagggcgagcggcggcggggcaaggggcacgacgggctgtaccaggggctgagcaccgcaacaaaggacacctatgatgccctgcacatgcaggctctgcccccccggtaatagaaaaacaaaaaacaaaaaccacacaaatggtcgccgaaactactgaaagcat

SEQ ID NO: 214 - RNA, 2299 nt, synthetic construct
  source 1..2299: mol_type = other RNA
aacttttagttattttcggcgaccatttgtgtggtgagctctaagtgttgatcagcgggcgcagctgcatatgacctctgcgtgtgcctggtaattgactaagatatctttgctcagcgtaacttctccgggttacgtggagaccaaaaggctacggagactcgggctacggccctggagcacctaggtgctcctaaagacgttagaagttgtacaaactcgcccaatagggccccccaaccaggggggtagcgggcaagcacttctgtttccccggtatgatctcataggctgtacccacggctgaaagagagattatcgttacccgcctcactacttcgagaagcccagtaatggttcatgaagttgatctcgttgacccggtgtttcccccacaccagaaacctgtgatgggggtggtcatcccggtcatggcgacatgacggacctccccgcgccggcacagggcctcttcggaggacgagtgacatggattcaaccgtgaagagcctattgagctagtgttgattcctccgcccccgtgaatgcggctaatcccaactccggagcaggcgggcccaaaccagggtctggcctgtcgtaacgcgaaagtctggagcggaaccgactactttcgggaaggcgtgtttccttttgttccttttatcaagttttatggtgacaactcctggtagacgttttattgcgtttattgagagatttccaacaattgaacagactagaaccacttgttttatcaaaccctcacagaataagataacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 215 - RNA, 2302 nt, synthetic construct
  source 1..2302: mol_type = other RNA
aacttttagttattttcggcgaccatttgtgtggtgagctctaagtgttgatcagcgggcgcagctgcatatgacctctgcgtgtgcctggtaattgactaagatatctgaaccgttacgcaccactcagttggtgtttggtggcaccaatgatggaacaaaaggctacaccacttgggctacggcccgcgccaccttgtggcgcaaagacattagaagaatagcataccgcccactagggccctgcagccagcagggtaacgggcaagcacttctgtctccccggtagaacggtataggctgtacccacggccgaaaactgaactatcgttacccgactccgtacttcgcaaagcttagtaggaaactggaaagttcgagttattgacccggagtgttccccccactccagaaacgcgtgatgagggttgccaccccgaccatggcgacatggtgggcatccctgcgctggcacgcggcctctaagaggataactcgctcctactggtaaccgaagagccccgtgagctacggtttattcctccgcctccctgaatgcggctaatcctaacccatgagcagttgccatagatccatatggtggactgtcgtaacgcgtaagttgtgggcggaaccgactactttgggatggcgtgtttccttgttttctccatttgttgttgtatggtgacaagttatagatctcgatctatagcgtttcttgagagatttccaaacatttattcaagtcgtacaattcttgtgtttaagcagtacagtgtaaggatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 216 - RNA, 2173 nt, synthetic construct
  source 1..2173: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcagagtgaagttcagcagaagcgctgacgcccctgcttaccagcagggacagaaccagctgtttaacgagctgaacctggggagaagagaagagttcgacgtgctggacaagcggagaggcagagatcctgagatgggcggcaagcccagaagaaagaaccctcaagagggcctgtttaatgagctgcagaaagacaagatggccgaggccttcagcgagatcggaatgaagggcgagcgcagaagaggcaagggacacgatggactgttccagggcctgagcacagccaccaaggacacattcgacgccctgcacatgcaggccctgcctccaagataatagaaaaacaaaaaacaaaaaccacacaaatggtcgccgaaactactgaaagcat

SEQ ID NO: 217 - RNA, 2365 nt, synthetic construct
  source 1..2365: mol_type = other RNA
aacttttagttattttcggcgaccatttgtgtggtgagctctaagtgttgatcagcgggcgcagctgcatatgacctctgcgtgtgcctggtaattgactaagatatcttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 218 - RNA, 2169 nt, synthetic construct
  source 1..2169: mol_type = other RNA
aacttttagttattttcggcgaccatttgtgtggtgagctctaagtgttgatcagcgggcgcagctgcatatgacctctgcgtgtgcctggtaattgactaagatatccccccttttccctaaccccaaccgccgtatctggtggcggcaagacacacgggtctttccctctaaagcacaaatgtgtgtgtgtcccaggtcctcctgcgctcggtgcgggagtgctcccacccaactgttgtaagcctgtccaacgtgtcgtcctggcaagactatgacgtcgcatgttccgctgcggatgccgaccgggtaaccggttccccagtgtgtgtagtgcgatcttccaggttcccctggttggcgttgtccagaaactgcttcaggtaagtggggtgtgcccaatccctgcaaaggttgattctctcaccaccttaggaatgctccggaggtaccccagcaacagctgggatctgaccggaggctaattgtctacgggtggtgtttcctttttctttttccacaacttcattgctgacaactcactgactacccatttgctctcttgtgcctttctgctctggttcaagctccttgattgtttttgattgctttctattgctttcttccactcaacccttgctcagttcaaagtcatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 219 - RNA, 2225 nt, synthetic construct
  source 1..2225: mol_type = other RNA
aacttttagttattttcggcgaccatttgtgtggtgagctctaagtgttgatcagcgggcgcagctgcatatgacctctgcgtgtgcctggtaattgactaagatatctttaagtgttgtgcccaatctcttgactcctgctggaaccaccgaccagtagtgtccaaaatgccaggtggaaaatcctcccttcccctctgggcttcatgcccggcatcctccccccagcctgacgtgccacaggctgtgcaaagaccccgcgaaagctgccaaaagtggcaattgtgggtcccccctttgtcaaggcgtcgagtctttctcccttaaggctagtcctgtcagtgaactctgtcgggcaactagtgacgccactgcatgcctccgacctcggccgcggagtgctgccccccaagtcatgcccctgaccacaagttgtgctgtctggcaaacattgtctgtgagaatgttccgctgtggctgccaagcctggtaacaggctgccccagtgtgcgtaattctcatccagacttcggtctggcaacttgctgttaagacatggcgtaaggggcgtgtgccaacgccctggaacgagtgtccactctaataccccgaggaatgctacgcaggtacccctggctcgccagggatctgagcgtaggctaattgtctaagggtattttcatttcccaccctcttcttcttgttcataatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 220 - RNA, 2236 nt, synthetic construct
  source 1..2236: mol_type = other RNA
aacttttagttattttcggcgaccatttgtgtggtgagctctaagtgttgatcagcgggcgcagctgcatatgacctctgcgtgtgcctggtaattgactaagatatctttcccctgttcgtaactaagtgtgtgcccaatctcctcactcctgctggcttcaccgaccggcagtgtccaaaatgctaggtgaatcccctccctttcctctgggcttctgcccagcttcctccccccagcctgacgtgacacaggctgtgcaaagaccccgcgaaagctgccaaaagtggcaattgtgggtcccccctttgtaaaggcgtcgagtctttctccctcaaggctagacccgtcagtgaattctgtcgggcaactagtgacgccactgcacgcctctgacctcggccgcggagtgctgccccccaagtcgtgcccctgaccacaagttgtgctgtctggcaaacattgtctgtgagaatgttccgctgtggctgccaagcctggcaacaggctgccccagtgtgcgtagttctcatccagacttcggtctggcaacttgctgttaagacacggcgtaaggggcgtgtgccaacgccctggaacgagtgtccactctaataccccgaggaatgctacgcaggtacccctggttcgccagggatctgagcgtaggctaattgtctaagggtattttcatttcccattctttctttcttgttcataatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 221 - RNA, 2322 nt, synthetic construct
  source 1..2322: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagacaacaacaaacaacaaaacaacaaacaacacaaaaccaaaccacacaaatggtcgccgaaactactgaaagcat

SEQ ID NO: 222 - RNA, 2229 nt, synthetic construct
  source 1..2229: mol_type = other RNA
aacttttagttattttcggcgaccatttgtgtggtgagctctaagtgttgatcagcgggcgcagctgcatatgacctctgcgtgtgcctggtaattgactaagatatcttcaagtgtgtgtgctcgtaatcttgactcctgccggaatgccgcccggttcagtgaacaaacagctaggcaagtccctcccttcccctgtggtcggttctcaccggccaccatccctcccccagcctgacgtgttacaggctgtgcaaagcccccgcgaaagctgctcacgtggcaattgtgggtcccccctttgtcaagacaccgagtctttctcccttaaggctagcccggtcccacgaacgtggaactggcaactagtggtgtcactacacgcctccgacctcggacgcggagtgctgttccccaagctgtaaccctgacccaagactgtgctgcctggcaagcaccgtctgggaagatgttccgctgtggctgccaaacctggtaacaggtgccccagtgtgtgtagtcttcctccagtctccggactggcagtcttgtgtaaagatgcagtgtaaggttcaagtgccaaatccctggaaggagtgaccctctactgccctaggaatgctgtgcaggtacccccaacttcggttggggatctgagcacaggctaattgtctacgggtagtttcatttcccatcctctcttttttggcatcatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 223 - RNA, 2365 nt, synthetic construct
  source 1..2365: mol_type = other RNA
aacttttagttattttcggcgaccatttgtgtggtgagctctaagtgttgatcagcgggcgcagctgcatatgacctctgcgtgtgcctggtaattgactaagatatctgtaggtagaaggtataaacgaccagccacacacttcctacccaaagcgccaataacaggagcccggattggtctggcacgcaaaggagtagacatcccccaatgttttccgaaccaagcggcagcagcgtcggttaggatcctcataattctgatcgaaggacagtacacgcgctactcagggtcttctatgatgattacgatttacttacgacggtgtgatgtaacgccctcggcgacttacttggacctgttcccgaattgagaatgtgtaggccagcgaccaatcgccaccacgaggcctagactgatcaacttcaactgtgagcgactcgcatctgaggagttgacctagctaacacttcccatccactctaatttggctaacaaggctgccacgtgatgatccttcttctagggtgtctacctgagtaagggtacccttctcggaataacgataagcgaaggaggacatgaacctaggctggatgtgattagctccgtaagctgccaacctggagcgcgtcagattcggccaacggcagattgggctgatcgggagtgaggaatccctgcttacgctcactgggtgattccacgtaaaatgtccagttattatctgtgtcctcttaacacattcttctgtttaagcttttcgcccgttctataaaccttgtctgactaagtccttagtccgctgccattaccgattgggttgcgtaggcagcttctattgaaacgatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 224 - RNA, 2289 nt, synthetic construct
  source 1..2289: mol_type = other RNA
aacttttagttattttcggcgaccatttgtgtggtgagctctaagtgttgatcagcgggcgcagctgcatatgacctctgcgtgtgcctggtaattgactaagatatcgcccgtccccctcaccctcttttccggtggccacgcccgggccaccgatacttcccttcactccctcgggactgttggggaggaacacaacagggctcccctgtatttcctcttcccattccccctttcctaaccccaaccgccgtatctggtggcggtaagacacacgggtctttccctctaaagcacaattgtgtgtgtgtcccaggtcctcctgcgttcggtgcgggagtgctcccacccaactgttgtaagcctgtccaacgtgtcgtcctggcaagactatgacgtcgcatgttccgctgtggatgccgaccgggtaaccggttccccagtgtgtgtagtgcgatcttccaggttctcctggttggcgttgtccagaaactgcttcgggtaagtggggtgtgcccaatccctacaagggttgattctttcaccaccttaggaatgctccggaggtaccccagcaacagctgggatctgaccggaggctaattgtctacgggtggtgtttccattttctttttcacacaacttcattgctgacaactcactgactaatcacttgctctcttgtgcctttctgctctggttcaagttccttgattgtttgtttgattgcttttcactgctttcttcccacaatccttgctcagttcaaagtcatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 225 - RNA, 2170 nt, synthetic construct
  source 1..2170: mol_type = other RNA
aacttttagttattttcggcgaccatttgtgtggtgagctctaagtgttgatcagcgggcgcagctgcatatgacctctgcgtgtgcctggtaattgactaagatatcttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtcatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 226 - RNA, 2243 nt, synthetic construct
  source 1..2243: mol_type = other RNA
aacttttagttattttcggcgaccatttgtgtggtgagctctaagtgttgatcagcgggcgcagctgcatatgacctctgcgtgtgcctggtaattgactaagatatcgtaacttcaagtgtgtgtgctcgtaatcttgactcctgccggaatgccgcccggttcagtgaacaaacagctaggcaagtccctcccttcccctgtggtcggttctcaccggccaccatccctcccccagcctgacgtgttacaggctgtgcaaagcccccgcgaaagctgctcacgtggcaattgtgggtcccccctttgtcaagacaccgagtctttctcccttaaggctagcccggtcccacgaacgtggaactggcaactagtggtgtcactacacgcctccgacctcggacgcggagtgctgttccccaagctgtaaccctgacccaagactgtgctgcctggcaagcaccgtctgggaagatgttccgctgtggctgccaaacctggtaacaggtgccccagtgtgtgtagtcttcctccagtctccggactggcagtcttgtgtaaagatgcagtgtaaggttcaagtgccaaatccctggaaggagtgaccctctactgccctaggaatgctgtgcaggtacccccaacttcggttggggatctgagcacaggctaattgtctacgggtagtttcatttcccatcctctcttttttggcatcatggcgaacatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 227 - RNA, 2405 nt, synthetic construct
  source 1..2405: mol_type = other RNA
aacttttagttattttcggcgaccatttgtgtggtgagctctaagtgttgatcagcgggcgcagctgcatatgacctctgcgtgtgcctggtaattgactaagatatcccccctcaccctcttttccggcggcgcatgttcgcgtcgtcgtaagtctggattcccaaggcccactcggttcaacttcggtttccggacaaatacaaagaacctcagtcctcttggtactttctcgcctgagtctacaaagcgagaaacctgcccctctaacgccagacgagcggcataaaactcgaacttctggcacgttccaccaccccttctcctatcccaacccccattgcgctctcaaggtcgcgcttttccgagactagctcggattcaaaaagttcctggcaccctttaccccttcaggcccttaaggtaggaactgaccttgtgctgtgatctcggtgcggaagtgctactgcgtagtgattgtaagatccttttgtggttctgccctggcaaggctacagagtgctgtgatccgctgcggatgccatcctggtaacaggacccccagtgtgcgcaacagtatgttcacggtcttccgtgttcaccacattcggaacactgctttcgtgaaacagtgtgtgtccaatccctgtgatcagtatcaaccacaccacctaggaatgctaggaaggtaccccggttcgccgggatctgatcctaggctaattgtctacggtggtgctcctttttattttccacttcaactcactggttacaactgcttgatttctgtgtttgctgcttttctctgctctcactgccattctcaagtgttcacactgtccaagctcctttggttgttcgcttccacttgccactgtcaactcttgtcatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacaaaaaacaaaaaccacacaaatggtcgccgaaactactgaaagcat

SEQ ID NO: 228 - RNA, 2329 nt, synthetic construct
  source 1..2329: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacacaaaccaaacaccattgtcacactccaaaaaaaacaaacaaccacacaaatggtcgccgaaactactgaaagcat

SEQ ID NO: 229 - RNA, 2424 nt, synthetic construct
  source 1..2424: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaaaaacacaaaccaaacaccattgtcacactccaactcatgatcagtacagaacaaacaccattgtcacactccaagctgacatacatatcataactctactatagacaaacaccattgtcacactccaaaaaaaacaaacaaccacacaaatggtcgccgaaactactgaaagcat

SEQ ID NO: 230 - RNA, 2353 nt, synthetic construct
  source 1..2353: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatgctgctgctggtgacaagcctgctgctgtgcgagctgccccaccccgcctttctgctgatccccgacatccagatgacccagaccaccagcagcctgagcgccagcctgggcgacagagtgaccatcagctgccgggccagccaggacatcagcaagtacctgaactggtatcagcagaaacccgacggcaccgtgaagctgctgatctaccacacctcccggctgcacagcggcgtgcccagcagattttctggcagcggcagcggcaccgactacagcctgaccatctccaacctggaacaagaagatatcgctacctatttttgtcaacaaggcaacaccctgccctacaccttcggcggaggcaccaaactggaaatcaccggcagcaccagcggctccggcaagcctggatctggcgagggcagcaccaagggcgaagtgaagctgcaagaaagcggccctggcctggtggcccctagccagagcctgtccgtgacctgtaccgtgtccggcgtgtccctgcccgactacggcgtgtcctggatccggcagccccccagaaagggcctggaatggctgggcgtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgtccagcttcgtgcccgtgtttctgcccgccaagcctaccaccacccctgcccctagacctcccaccccagccccaacaatcgccagccagcctctgtccctgcggcccgaagcctgtagaccagctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgataatagaaaaacaaaaaacaaaaaccacacaaatggtcgccgaaactactgaaagcat

SEQ ID NO: 231 - RNA, 2308 nt, synthetic construct
  source 1..2308: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggcattaccagttacagccctcctcctccctctcgctctcctcctccacgccgccaggccagatatccagatgacacagaccaccagcagcctcagcgccagcctcggcgatagagtgacaatctcttgcagagcctcccaggacatcagcaagtacctcaattggtaccagcagaagcccgacggcaccgtgaagctcctgatctaccacaccagcagactgcacagcggcgtgccttctagattctctggaagcggcagcggcaccgactacagcctgaccatcagcaacctggagcaggaggacatcgccacctacttctgccagcagggcaataccctgccctacaccttcggcggcggcacaaaactggaaattacaggtggtggtggttctgggggaggagggtctggtggtggtggtagtgaagtgaaactgcaggaatctggaccaggactggtggccccatctcagtctctgagcgtgacatgtacagtgtctggcgtgtctctgcctgattacggagtgtcttggattagacagcctcctcggaagggcctggagtggctgggagtgatctggggcagcgagaccacctactacaacagcgccctgaagtccaggctgaccatcatcaaggacaacagcaagtcccaggtgttcctgaagatgaacagcctgcagaccgacgacaccgccatctactactgcgccaagcactactactacggcggcagctacgccatggattactggggccagggcaccagcgtgacggtgtcttctacaacaacacctgctcctaggcctcctacaccagctcctacaattgcttctcagccgctgtctctgcggccagaagcttgtaggcctgctgcaggaggagcggtgcatacaagaggactggactttgcctgtgatatctacatctgggcccctctggccggcacctgcggcgtgctgctgctgagcctggtgatcaccctgtactgcaaccacagaaacagaagcaagagaagcaggctgctgcacagcgactacatgaatatgacacctaggagacctggccctacaagaaaacattatcagccttatgcccctcctagagattttgccgcctatagaagccgggtgaagttttctagatctgctgatgccccagcctatcagcagggccagaaccagttatataatgaactgaacctgggccggcgggaggagtacgatgtgctggataagagaagaggcagagatcctgaaatgggaggaaaacctagaagaaagaatccccaggagggcctatataacgaactgcagaaggacaagatggccgaggcctacagcgagatcggcatgaagggcgagagaagaagaggcaagggccacgatggactgtatcaggggctgagcaccgccaccaaggatacctacgatgccctgcacatgcaggccctgcctcctagataatagaaaaacaaaaaacaaaaaccacacaaatggtcgccgaaactactgaaagcat

SEQ ID NO: 232 - RNA, 2308 nt, synthetic construct
  source 1..2308: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatgctattattagtcaccagcctcctcctttgcgagctcccccaccccgccttcctcctcatccccgacatccagatgacccagaccaccagcagcctcagcgccagcctcggcgatagagtgacaatctcttgcagagcctcccaggacatcagcaagtacctcaattggtaccagcagaagcccgacggcaccgtgaagctgctgatctaccacaccagcagactgcacagcggcgtgccttctagattctctggaagcggcagcggcaccgactacagcctgaccatcagcaacctggagcaggaggacatcgccacctacttctgccagcagggcaataccttgccctacaccttcggcggcggcacaaagctggaaattacaggtggtggtggtagtggtggtggtggttctggtggtggtggatctgaagtgaaactgcaggaatctggacctggcctggtggccccttctcagtcgctgagcgtgacatgtacagtgagcggcgtgagcctgcctgattatggagtgagctggatcagacagcctccaagaaagggcctggagtggctgggcgtgatctggggctccgagaccacctactacaacagcgccctgaagtccaggctgaccatcatcaaggacaacagcaagagccaggtgttcctgaagatgaacagcctgcagaccgatgacaccgccatctactactgcgccaagcactactactacggcggcagctacgccatggattactggggccagggcacatctgtgacagtgtcttctgctgctgctccagctcctagacctcctacacctgctcctacaattgcttctcagccgctgagcctgagacctgaagcttgtagacctgctgctggaggagctgtgcatacaagaggactggattttgataccgccctggccgccgtgatctgttctgccctggccacagtgctgctggccctgctgatcctgtgcgtgatctactgcaagagacagcccagaagaaagaagctgctgtacatcttcaagcagcccttcatgagacctgtgcagacaacacaggaggaggatggctgttcctgtagatttccagaggaagaggagggcggctgcgagctgagagtgaaattttctagatctgccgatgcccctgcctaccagcagggccagaaccagctatataatgaactgaacctgggcagaagagaggagtacgacgtgctggataagagaagaggcagagatcctgaaatgggaggaaaacctagaagaaagaatccgcaggagggcctatataacgaactgcagaaggacaagatggccgaggcctacagcgagatcggcatgaagggcgagagaagaagaggcaagggccacgatggactgtatcaggggctgagcacagccaccaaggatacctacgatgccctgcacatgcaggccctgcctcctagataatagaaaaacaaaaaacaaaaaccacacaaatggtcgccgaaactactgaaagcat

SEQ ID NO: 233 - RNA, 2296 nt, synthetic construct
  source 1..2296: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctacccgttacagcattactactacctcttgccctcctcctccacgccgccagaccagatatccagatgacccagaccacctctagcctcagcgccagcctcggcgatagagtgacaatctcttgcagagcctcccaggacatcagcaagtacctcaattggtaccagcagaagcccgacggcaccgtgaagctcctgatctaccacaccagcaggctgcactctggcgtgccatctagattttctggaagcggcagcggcacagattacagcctgaccatcagcaatctggagcaggaggacatcgccacctacttctgccagcagggcaataccctgccctacaccttcggcggcggcacaaaactggaaattacaggtggtggtggttcagggggaggagggtctggtggtggaggttctgaagtgaaactgcaggaatctggacctggcctggtggccccttctcagtcgctgagcgtgacatgtaccgtgagcggcgtgagcctgcctgattacggggtgagctggatcagacagccccctagaaagggcctggaatggctgggcgtgatctggggcagcgagaccacctactacaacagcgccctgaagtccaggctgaccatcatcaaggacaacagcaagtcccaggtgttcctgaagatgaacagcctgcagaccgacgacaccgccatctactactgcgccaagcactactactacggcggcagctacgccatggattactggggccagggcacctctgtgacagtgagcagcgccgctgccattgaggtgatgtaccctcccccctacctggacaacgagaagtccaacggcaccatcatccacgtgaagggcaagcacctgtgccctagccccctgtttccaggcccatctaagcctttttgggtgctggtggtggtgggcggcgtgctggcctgctacagcctgctggtgaccgtggccttcatcatcttctgggtgagaagcaagaggtcccggctgctgcacagcgactacatgaatatgacacccaggagacctggccctacaagaaaacattatcagccttatgcccctcctagagattttgccgcctatagaagccgggtgaagttttctagaagcgctgatgccccagcctaccagcagggccagaaccagctgtacaacgagctgaacctgggcagaagagaggagtacgacgtgctggataagagaagaggcagagatccggaaatgggaggaaagcctagaagaaagaatcctcaggagggcctgtacaatgagctgcagaaggacaagatggccgaggcctactctgagatcggcatgaagggcgagagaagaagaggcaagggccacgatggcctgtaccagggcctgagcacggccaccaaggatacctacgatgccctgcacatgcaggccctgcctcctagataatagaaaaacaaaaaacaaaaaccacacaaatggtcgccgaaactactgaaagcat

SEQ ID NO: 234 - protein, 5 aa, synthetic construct
TYTMG

SEQ ID NO: 235 - protein, 17 aa, synthetic construct
AISWSAGRTYYADSMKG

SEQ ID NO: 236 - protein, 17 aa, synthetic construct
DPWTSDSDYDRLTMYDY

SEQ ID NO: 237 - protein, 5 aa, synthetic construct
MYSMS

SEQ ID NO: 238 - protein, 17 aa, synthetic construct
SISTGARDTAYADSVKG

SEQ ID NO: 239 - protein, 12 aa, synthetic construct
GDLRYGPDGYDY

SEQ ID NO: 240 - protein, 5 aa, synthetic construct
VDATT

SEQ ID NO: 241 - protein, 16 aa, synthetic construct
IMDIGGVTEYADSVKG

SEQ ID NO: 242 - skipped

SEQ ID NO: 243 - protein, 5 aa, synthetic construct
TNNMA

SEQ ID NO: 244 - protein, 16 aa, synthetic construct
LIRTGGSTHVADSMKG

SEQ ID NO: 245 - protein, 11 aa, synthetic construct
WTGSPGALSDY

SEQ ID NO: 246 - protein, 5 aa, synthetic construct
NYAAG

SEQ ID NO: 247 - protein, 17 aa, synthetic construct
RISRSGGRTDYADSVKG

SEQ ID NO: 248 - protein, 15 aa, synthetic construct
ATVWEFTDGADQYDY

SEQ ID NO: 249 - protein, 5 aa, synthetic construct
TYGMG

SEQ ID NO: 250 - protein, 17 aa, synthetic construct
VITGSGVGTQYADSVKD

SEQ ID NO: 251 - protein, 16 aa, synthetic construct
GHRPGWAVIRADAYEY

SEQ ID NO: 252 - protein, 5 aa, synthetic construct
RVGMN

SEQ ID NO: 253 - protein, 17 aa, synthetic construct
SISSDGSRTNYAHFVKG

SEQ ID NO: 254 - protein, 4 aa, synthetic construct
PGNS

SEQ ID NO: 255 - protein, 5 aa, synthetic construct
SSFMS

SEQ ID NO: 256 - protein, 16 aa, synthetic construct
TIYSDGSTYYADSVKG

SEQ ID NO: 257 - protein, 5 aa, synthetic construct
VTGSI

SEQ ID NO: 258 - protein, 5 aa, synthetic construct
TYNMG

SEQ ID NO: 259 - protein, 17 aa, synthetic construct
AIDLYGRATRYANSVKG

SEQ ID NO: 260 - protein, 18 aa, synthetic construct
DTSLPLGVLTESQRLYGA

SEQ ID NO: 261 - protein, 5 aa, synthetic construct
DYGVS

SEQ ID NO: 262 - protein, 16 aa, synthetic construct
VIWGSETTYYNSALKS

SEQ ID NO: 263 - RNA, 741 nt, synthetic construct
  source 1..741: mol_type = other RNA
tgtaggtagaaggtataaacgaccagccacacacttcctacccaaagcgccaataacaggagcccggattggtctggcacgcaaaggagtagacatcccccaatgttttccgaaccaagcggcagcagcgtcggttaggatcctcataattctgatcgaaggacagtacacgcgctactcagggtcttctatgatgattacgatttacttacgacggtgtgatgtaacgccctcggcgacttacttggacctgttcccgaattgagaatgtgtaggccagcgaccaatcgccaccacgaggcctagactgatcaacttcaactgtgagcgactcgcatctgaggagttgacctagctaacacttcccatccactctaatttggctaacaaggctgccacgtgatgatccttcttctagggtgtctacctgagtaagggtacccttctcggaataacgataagcgaaggaggacatgaacctaggctggatgtgattagctccgtaagctgccaacctggagcgcgtcagattcggccaacggcagattgggctgatcgggagtgaggaatccctgcttacgctcactgggtgattccacgtaaaatgtccagttattatctgtgtcctcttaacacattcttctgtttaagcttttcgcccgttctataaaccttgtctgactaagtccttagtccgctgccattaccgattgggttgcgtaggcagcttctattgaaacg

SEQ ID NO: 264 - RNA, 2332 nt, synthetic construct
  source 1..2332: mol_type = other RNA
aacttttagttattttcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatccagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaacaaacacaaaccaaacaccattgtcacactccaaacaacaaacaaacaaccacacaaatggtcgccgaaactactgaaagcat

SEQ ID NO: 265 - RNA, 108 nt, synthetic construct; identical to SEQ ID NO: 43
  source 1..108: mol_type = other RNA

SEQ ID NO: 266 - RNA, 1458 nt, synthetic construct
  source 1..1458: mol_type = other RNA
atggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatccagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaaga

SEQ ID NO: 267 - RNA, 50 nt, synthetic construct
  source 1..50: mol_type = other RNA
aacaaacacaaaccaaacaccattgtcacactccaaacaacaaacaaaca

SEQ ID NO: 268 - RNA, 91 nt, synthetic construct
  source 1..91: mol_type = other RNA
taatagaacaaacacaaaccaaacaccattgtcacactccaaacaacaaacaaacaaccacacaaatggtcgccgaaactactgaaagcat

SEQ ID NO: 269 - RNA, 2329 nt, synthetic construct; identical to SEQ ID NO: 228
  source 1..2329: mol_type = other RNA

SEQ ID NO: 270 - RNA, 47 nt, synthetic construct
  source 1..47: mol_type = other RNA
aaaaacacaaaccaaacaccattgtcacactccaaaaaaaacaaaca

SEQ ID NO: 271 - RNA, 91 nt, synthetic construct; identical to SEQ ID NO: 268
  source 1..91: mol_type = other RNA

SEQ ID NO: 272 - RNA, 88 nt, synthetic construct; identical to SEQ ID NO: 46
  source 1..88: mol_type = other RNA

SEQ ID NO: 273 - RNA, 282 nt, synthetic construct
  source 1..282: mol_type = other RNA
aactactgaaagcataaataattgtgcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctattcgtaacataattgaacttttagttattt

SEQ ID NO: 274 - RNA, 1774 nt, synthetic construct
  source 1..1774: mol_type = other RNA
aggaaataagagagaaaagaagagtaagaagaaatataagagccaccatgctgctgctggtgacaagcctgctgctgtgcgagctgccccaccccgcctttctgctgatccccgacatccagatgacccagaccaccagcagcctgagcgccagcctgggcgacagagtgaccatcagctgccgggccagccaggacatcagcaagtacctgaactggtatcagcagaaacccgacggcaccgtgaagctgctgatctaccacacctcccggctgcacagcggcgtgcccagcagattttctggcagcggcagcggcaccgactacagcctgaccatctccaacctggaacaagaagatatcgctacctatttttgtcaacaaggcaacaccctgccctacaccttcggcggaggcaccaaactggaaatcaccggcagcaccagcggctccggcaagcctggatctggcgagggcagcaccaagggcgaagtgaagctgcaagaaagcggccctggcctggtggcccctagccagagcctgtccgtgacctgtaccgtgtccggcgtgtccctgcccgactacggcgtgtcctggatccggcagccccccagaaagggcctggaatggctgggcgtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgtccagcttcgtgcccgtgtttctgcccgccaagcctaccaccacccctgcccctagacctcccaccccagccccaacaatcgccagccagcctctgtccctgcggcccgaagcctgtagaccagctgccggcggagccgtgcacaccagaggcctggacttcgcctgcgacatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccagatgataataggctggagcctcggtggccatgcttcttgccccttgggcctccccccagcccctcctccccttcctgcacccgtacccccgtggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 275 - RNA, 1723 nt, synthetic construct
  source 1..1723: mol_type = other RNA
aggaaataagagagaaaagaagagtaagaagaaatataagagccaccatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatctagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagatgataataggctggagcctcggtggccatgcttcttgccccttgggcctccccccagcccctcctccccttcctgcacccgtacccccgtggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 276 - RNA, 164 nt, synthetic construct
  source 1..164: mol_type = other RNA
aataagagaaagtgcaacgactattccgataggaagtagggtcaagtgactcgaaatggggattacccttctagggtagtgatatagtctgaacatatatggaaacatatagaaggataggagtaacgaacctattcgtaacataattgaacttttagttattt

SEQ ID NO: 277 - RNA, 118 nt, synthetic construct
  source 1..118: mol_type = other RNA
aactactgaaagcataaataattgtgcctttatacagtaatgtatatcgaaaaatcctctaattcagggaacacctaaacaaactaagatgtaggcaatcctgagctaagctcttagt

SEQ ID NO: 278 - RNA, 169 nt, synthetic construct
  source 1..169: mol_type = other RNA
tattccgataggaagtagggtcaagtgactcgaaatggggattacccttctagggtagtgatatagtctgaacatatatggaaacatatagaaggataggagtaacgaacctattcgtaacataattgaacttttagttatttgagctctcggcgaccatttgtgtggt

SEQ ID NO: 279 - RNA, 139 nt, synthetic construct
  source 1..139: mol_type = other RNA
aactactgaaagcataaataattgtgcctttatacagtaatgtatatcgaaaaatcctctaattcagggaacacctaaacaaactaagatgtaggcaatcctgagctaagctcttagtaataagagaaagtgcaacgac

SEQ ID NO: 280 - RNA, 135 nt, synthetic construct
  source 1..135: mol_type = other RNA
taggaagtagggtcaagtgactcgaaatggggattacccttctagggtagtgatatagtctgaacatatatggaaacatatagaaggataggagtaacgaacctattcgtaacataattgaacttttagttattt

SEQ ID NO: 281 - RNA, 147 nt, synthetic construct
  source 1..147: mol_type = other RNA
aactactgaaagcataaataattgtgcctttatacagtaatgtatatcgaaaaatcctctaattcagggaacacctaaacaaactaagatgtaggcaatcctgagctaagctcttagtaataagagaaagtgcaacgactattccga

SEQ ID NO: 282 - protein, 21 aa, synthetic construct
MALPVTALLLPLALLLHAARP

SEQ ID NO: 283 - protein, 22 aa, synthetic construct
MLLLVTSLLLCELPHPAFLLIP

SEQ ID NO: 284 - protein, 45 aa, synthetic construct
TTTPAPRPPTPAPTIASQPLSLRPEACRPAAGGAVHTRGLDFACD

SEQ ID NO: 285 - protein, 39 aa, synthetic construct
PAPRPPTPAPTIASQPLSLRPEACRPAAGGAVHTRGLDF

SEQ ID NO: 286 - protein, 40 aa, synthetic construct
IEVMYPPPYLDNEKSNGTIIHVKGKHLCPSPLFPGPSKPF

SEQ ID NO: 287 - protein, 21 aa, synthetic construct
IYIWAPLAGTCGVLLLSLVIT

SEQ ID NO: 288 - protein, 26 aa, synthetic construct
WVLVVVGGVLACYSLLVTVAFIIFWV

SEQ ID NO: 289 - protein, 30 aa, synthetic construct
DTALAAVICSALATVLLALLILCVIYCKRQ

SEQ ID NO: 290 - protein, 10 aa, synthetic construct
FVPVFLPAKP

SEQ ID NO: 291 - protein, 41 aa, synthetic construct
RSKRSRLLHSDYMNMTPRRPGPTRKHYQPYAPPRDFAAYRS

SEQ ID NO: 292 - protein, 42 aa, synthetic construct
KRGRKKLLYIFKQPFMRPVQTTQEEDGCSCRFPEEEEGGCEL

SEQ ID NO: 293 - protein, 38 aa, synthetic construct
KKLLYIFKQPFMRPVQTTQEEDGCSCRFPEEEEGGCEL

SEQ ID NO: 294 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - DNA, 25 bp, synthetic construct
  source 1..25: mol_type = other DNA
aaaaaaaaaaaaaaaaaaaaaaaaa

SEQ ID NO: 296 - DNA, 675 bp, synthetic construct
  source 1..675: mol_type = other DNA
ttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattaca

SEQ ID NO: 297 - DNA, 1458 bp, synthetic construct
  source 1..1458: mol_type = other DNA
atggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatccagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaaga

SEQ ID NO: 298 - DNA, 22 bp, synthetic construct
  source 1..22: mol_type = other DNA
caaacaccattgtcacactcca

SEQ ID NO: 299 - RNA, 51 nt, synthetic construct
  source 1..51: mol_type = other RNA
atggataactgaatctctttcaactaataagagacattatcacgaaacaat

SEQ ID NO: 300 - RNA, 27 nt, synthetic construct
  source 1..27: mol_type = other RNA
tatttctactttatttctacactgtta

SEQ ID NO: 301 - RNA, 30 nt, synthetic construct
  source 1..30: mol_type = other RNA
tttgccttgactgttagctgttacctgtta

SEQ ID NO: 302 - RNA, 49 nt, synthetic construct
  source 1..49: mol_type = other RNA
ctatttcactaaaattagatgaggtgcttgcactattcactgcacagag

SEQ ID NO: 303 - RNA, 40 nt, synthetic construct
  source 1..40: mol_type = other RNA
ctccctctctccctctcttccttcctctctctctttctct

SEQ ID NO: 304 - RNA, 20 nt, synthetic construct
  source 1..20: mol_type = other RNA
ctccctctctccctctcttc

SEQ ID NO: 305 - RNA, 20 nt, synthetic construct
  source 1..20: mol_type = other RNA
ttctcctcctttctttcctc

SEQ ID NO: 306 - RNA, 40 nt, synthetic construct
  source 1..40: mol_type = other RNA
ccttaccctttccccttcctccaatccctttccccttcca

SEQ ID NO: 307 - RNA, 20 nt, synthetic construct
  source 1..20: mol_type = other RNA
ccttaccctttccccttcct

SEQ ID NO: 308 - RNA, 20 nt, synthetic construct
  source 1..20: mol_type = other RNA
ccaatccctttccccttcca

SEQ ID NO: 309 - RNA, 63 nt, synthetic construct
  source 1..63: mol_type = other RNA
caaaaataaacaaataaattttaaaaataaataaataataaaaggaagtgttagacaatgtaa

SEQ ID NO: 310 - RNA, 52 nt, synthetic construct
  source 1..52: mol_type = other RNA
gagaatgtaagaactacaaacaaaatttctattaaattaaattttgtgtctc

SEQ ID NO: 311 - RNA, 19 nt, synthetic construct
  source 1..19: mol_type = other RNA
actcatgatcagtacagaa

SEQ ID NO: 312 - RNA, 32 nt, synthetic construct
  source 1..32: mol_type = other RNA
agctgacatacatatcataactctactataga

SEQ ID NO: 313 - RNA, 50 nt, synthetic construct
  source 1..50: mol_type = other RNA
gtaagcctcatttaattacactgttgtaatcgttattatacttttaagta

SEQ ID NO: 314 - RNA, 50 nt, synthetic construct
  source 1..50: mol_type = other RNA
ggtgcggcccgaagcgagtatcacctcacgatccaaacgacctctaagac

SEQ ID NO: 315 - RNA, 20 nt, synthetic construct
  source 1..20: mol_type = other RNA
accaaacaacaacacaaaaa

SEQ ID NO: 316 - RNA, 25 nt, synthetic construct
  source 1..25: mol_type = other RNA
caacaaaaaacaaacaaaaaaaaca

SEQ ID NO: 317 - RNA, 17 nt, synthetic construct
  source 1..17: mol_type = other RNA
aaaaacaaaaaacaaaa

SEQ ID NO: 318 - RNA, 663 nt, synthetic construct
  source 1..663: mol_type = other RNA
ggcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 319 - RNA, 544 nt, synthetic construct
  source 1..544: mol_type = other RNA
cccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 320 - RNA, 662 nt, synthetic construct
  source 1..662: mol_type = other RNA
gcctcggccccctcaccctcttttccggtggccacgcccgggccaccgatacttcccttcactccttcgggactgttggggaggaacacaacagggctcccctgttttcccattccttcccccttttcccaaccccaaccgccgtatctggtggcggcaagacacacgggtctttccctctaaagcacaattgtgtgtgtgtcccaggtcctcctgcgtacggtgcgggagtgctcccacccaactgttgtaagcctgtccaacgcgtcgtcctggcaagactatgacgtcgcatgttccgctgcggatgccgaccgggtaaccggttccccagtgtgtgtagtgcgatcttccaggtcctcctggttggcgttgtccagaaactgcttcaggtaagtggggtgtgcccaatccctacaaaggttgattctttcaccaccttaggaatgctccggaggtaccccagcaacagctgggatctgaccggaggctaattgtctacgggtggtgtttcctttttcttttcacacaactctactgctgacaactcactgactatccacttgctctcttgtgcctttctgctctggttcaagttccttgattgtttttgactgcttttcactgcttttcttctcacaatccttgctcagttcaaagtc

SEQ ID NO: 321 - RNA, 745 nt, synthetic construct
  source 1..745: mol_type = other RNA
ttaaaacagctctggggttgttcccaccccagaggcccacgtggcggctagtactctggtatcacggtacctttgtacgcctgttttatatcccttcccccgtaacttagaagctttgtaccaacaagttcaatagaaggggtacaaaccagtaccaccacgaacaagcacttctgtttccccggtgcgatgacatagactgtacccacggtcaaaagtcaccaatccgttacccgctttggtacttcgagacgcctagtattgccttggaattctcgatgcgttgcgctcaacactcgaccctggagtgtagcttaggctgatgagtctgggcattccccaccggtgacggtggcccaggctgcgttggcggcctacccatagctattgctatgggatgctagagttgaacaaggtgtgaagagccaattgagctactaaagagtcctccggcccctgaatgcggctaatcccaaccacggatcaagtgcgtgcaacccagcacgtagcttgtcgtaatgcgcaagtctgtggcggaaccgactactttgggtgtccgtgtttccttttatttttatcatggctgcttatggtgacaatcttggattgttatcatatagcgattggattggccatccggtgagaataaaagatattgtttatctgtttgtggcttttactccattaaacaaaaacacactttccttaattaaagtggtactattgatcagatcagcataccaca

SEQ ID NO: 322 - RNA, 600 nt, synthetic construct
  source 1..600: mol_type = other RNA
gtgtgtgtgctcgtaatcttgactcctgccggaatgccgcccggttcagtgaacaaacagctaggcaagtccctcccttcccctgtggtcggttctcaccggccaccatccctcccccagcctgacgtgttacaggctgtgcaaagcccccgcgaaagctgctcacgtggcaattgtgggtcccccctttgtcaagacaccgagtctttctcccttaaggctagcccggtcccacgaacgtggaactggcaactagtggtgtcactacacgcctccgacctcggacgcggagtgctgttccccaagctgtaaccctgacccaagactgtgctgcctggcaagcaccgtctgggaagatgttccgctgtggctgccaaacctggtaacaggtgccccagtgtgtgtagtcttcctccagtctccggactggcagtcttgtgtaaagatgcagtgtaaggttcaagtgccaaatccctggaaggagtgaccctctactgccctaggaatgctgtgcaggtacccccaacttcggttggggatctgagcacaggctaattgtctacgggtagtttcatttcccatcctctcttttttggcatc

SEQ ID NO: 323 - RNA, 552 nt, synthetic construct
  source 1..552: mol_type = other RNA
ttttgcggctctgccgccgttcgggttttacctgttttcacagagcaaaacaggacctctagtttcgtgcttaaacgagatcatgctcgaactagaactataacgctggtcactggacccgtgccgcgccttgcggatctttgcgggaatggtggctagtgggctgtggaagtgactctaaccacacgcccctcaagtgtgggaaaacacgaactggtgtagcgacgacgataggccttgggacaccctctccagtgatggagacccaaggggccaaaagccacgccttgtgccctgtcgttcacaaccccagtgcagttcgtgccagtacctgcttttgggaagtgtgctttggacagctgaaaacagtcctagtgggagactaaggatgcccaggaggtacccggaggtaacaagtgacactctggatctgacttggggagagcgggtctgctttacagacgccactctttaaaaaacttctatgtctcgtcaggcaccggaggccgggccttttcctttaaaacaatacactttatgaagacaacaata

SEQ ID NO: 324 - RNA, 629 nt, synthetic construct
  source 1..629: mol_type = other RNA
cacccatacacccccacccccttttctgtaactcaagtatgtgtgctcgtaatcttgactcccacggaatggatcgatccgctggagaacaaactgctagatccacatcctccctccccttgggaggacctcggtcctcccacatcctccctccagcctgacgtatcacaggctgtgtgaagcccccgcgaaagctgctcacgtggcaattgtgggtccccccttcatcaagacaccaggtctttcctccttaaggctagccccgatgtgtgaattcacattgggcaactagtggtgtcactgtgcgctcccaatctcggccgcggagtgctgttccccaagccaaacccctggcccttcactatgtgcctggcaagcatatctgagaaggtgttccgctgtggctgccagcctggtaacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccaccttcgggtgggatctgagcctgggctaattgtctacgggtagtttcatttccaattcttttatgctggagtc

SEQ ID NO: 325 - RNA, 770 nt, synthetic construct
  source 1..770: mol_type = other RNA
cacccacttttccggcggcgcacgttcgcgtcgtcgtaagtctggactcccaaggccaactcggttcaacttcggtttccggacaaatacaagaacctcagtcctcttggtactttctcgcctgagtcaacaaagcgagaaacctgcccctctaacgccagacgagcggcatgaaacttgaacttctggcatgttccactttccctctccctcaccccaccccaccgcgctctcaaggtcgcgctcttccgggactagcccggttttaaaagttcctggcaccctttgcccctctaggcccttaaggtaggaactgaccttgtgctgtgatctcggtgcggaagtgctactgcgtagtgattgtaagatccctttgtggttctgccctggcaaggctacagagtactgtgatccgctgcggacgccatcctggtaacaggacccccagtgtgcgcaacagtatgtccacggtcttccgtgttcaccacattcggaacactgctctcgtgaaacagtgtgtgtccaatccctgtgaccagtattaaccacaccacctaggaatgctaggaaggtaccccggttcgccgggatctgatcctaggctaattgtctacagtggtgctccttttattttcccctcacttcattgactacaactgcttgatttccgtgtttgctgctcttcttagctctcactgccattctcaagtgttcacactgtccaagctcctttggttgtttgcctccacttgccactgtcaactcttgtc

SEQ ID NO: 326 - RNA, 711 nt, synthetic construct
  source 1..711: mol_type = other RNA
ttaaaacagctctggggttgctcccaccccagaggcccacgtggcggctagtaatctggtatcaggtacctttgtacgcctgttttatatcccttcccccgtaactttagaagcttatcaaaagttcaatagcaggggtacaagccagtacctctacgaacaagcacttctgtttccccggtgaaatcatatagactgtacccacggtcaaaagtgattgatccgttatccgcttgagtacttcgagaagcctagtatcgccttggaatcttcgacgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaatcgctcacgacccagtgagtaggttgtcgtaatgcgtaagtctgtggcggaaccgactactttgggtgtccgtgtttccctttatattcatactggctgcttatggtgacaatttacgaattgttaccatatagctattggattggccacccagtgctgtgcaatatatttgagtgcttctttcataggtgttaccaacatcacatttaaaccacaatagtcagtgcaa

SEQ ID NO: 327 - RNA, 813 nt, synthetic construct
  source 1..813: mol_type = other RNA
cctagcccctttctgtaacggtctgtgtgagacctaaaaccacacataatatcgtgcctgatggttaacgcttgttagccgtctgtaaactaagaacctaccaacacactatgcacctcttggcgaagccgcttggaataagttgagaggaattatgcatgctagttgtgtttgtttacaactaattgttctaatccaagtgaagctcttcgcttggggcggcacgacacttgccgtaattcttctaccgtccctccacaccttgtggatgaagggccggatgtgtggcctctggctaacccctctctctggggtgatgctactggatgttttactcctagaccaaatcacatgaactcctcttgatccacttcggtggggctatgagcctgcggattaatagctggcgacagctaccccaggggccaaaagccacggtgttagcagcaccctcatagtctgatgcccaagggctgatgttgggagctagtagtgtgtgtctggcctatgtttaggacttctggccaagcgcagaggagtggggctgaaggatgcccagaaggtacccgtaggtaaccttaagagactatggatctgatctggggccccctcacgtggctttaccacgtgttgggggttaaaaaacgtctaggccccaccagcccacgggagtgggctttcccttaaaaagcccaacaatatttatggtgacaattcactgtttcttctttgcaatttttgtattcttggactccttattttttgtttgcttgattttagtggacattcagattcaaatacaaaatggcagga

SEQ ID NO: 328 - RNA, 730 nt, synthetic construct
  source 1..730: mol_type = other RNA
attcccaaggcccactcggttcaacttcggtttccggacaaatacaaagaacctcagtcctcttggtactttctcgcctgagtctacaaagcgagaaacctgcccctctaacgccagacgagcggcataaaactcgaacttctggcacgttccaccaccccttctcctatcccaacccccattgcgctctcaaggtcgcgcttttccgagactagctcggattcaaaaagttcctggcaccctttaccccttcaggcccttaaggtaggaactgaccttgtgctgtgatctcggtgcggaagtgctactgcgtagtgattgtaagatccttttgtggttctgccctggcaaggctacagagtgctgtgatccgctgcggatgccatcctggtaacaggacccccagtgtgcgcaacagtatgttcacggtcttccgtgttcaccacattcggaacactgctttcgtgaaacagtgtgtgtccaatccctgtgatcagtatcaaccacaccacctaggaatgctaggaaggtaccccggttcgccgggatctgatcctaggctaattgtctacggtggtgctcctttttattttccacttcaactcactggttacaactgcttgatttctgtgtttgctgcttttctctgctctcactgccattctcaagtgttcacactgtccaagctcctttggttgttcgcttccacttgccactgtcaactcttgtc

SEQ ID NO: 329 - RNA, 830 nt, synthetic construct
  source 1..830: mol_type = other RNA
cccccctcccccccttcccttccctttgcaacgcaacaattgtaagtgccctcacctgtcaattgggaccaccactttcagtgaccccatgcgaagtgctgagagaaaggaagctttcttacccttcatttgtgaacccactggtctaagccgcttggaatacgatgagtggaaaagttcattcttaatggagtgaaacatgcttaaatttccagctcgtgctggtctttccagtacggggcggccctgtctggccgtaattcttcagagtgtcacgccacacttgtggatctcacgtgccacatgacagcgctacagctggaactgggtgcttggtgcccatggagtaacagcgaaaagtgttagatcaagccttgcttgggctatgagcctgcggaacaacaactggtaacagttgcctcaggggccgaaagccacggtgttaacagcaccctcatagtttgatccacctcagggtggtgatgtttagcagttagtagttgccaatctgtgttcactgaaatctcggcataccgtgtagtgtacaggggtgaaggatgcccagaaggtacccgtaggtaaccttaagagactatggatctgatctggggccttgtccggagtgctttacacacggctcaaggttaaaaaacgtctagccccacagagcccgagggattcgggttttccctttaaaaacccgactagagcttatggtgacaattattgctgttcagacgaacagtgtaattgttgtctattcacagcagttctatcagagcttttcccacaacggatcttcttggcaagcaaatacagcaggagtcaat

SEQ ID NO: 330 - RNA, 741 nt, synthetic construct
  source 1..741: mol_type = other RNA
ttaaaacagcctgtgggttgcacccacccacagggcccactgggcgccagcactctggtatcgcggtacctttgtgcgcctgttttatgacccctccccaaccgaaacttagaagttacacataccgatcaatagtgggcatagcacgccagctgtgtcttgatcaagcacttctgtttccccggaccgagtatcaatagactgctcacgcggttgaaggagaaagcgttcgttatccggctaactacttcgagaaacctagtaacaccatgaaagttgcggagtgtttcgctcagcacttccccagtgtagatcaggtcgatgagtcaccgcgttccccacgggcgaccgtggcggtggctgcgctggcggcctgcctatgggatgacccataggacgcttcaacacagacatggtgtgaagagtctattgagctagttagtagtcctccggcccctgaatgcggctaatcctaactgcggagcacgtgccctcaagccagagggtggcgtgtcgtaacgggcaactctgcagcggaaccgactactttgggagtccgtgtttccttttattcttacactggctgcttatggtgacaattgagagattgttaccatatagctattggattggccatccggtgagcaacagagcgattgtatactcttttgtgggttttgtgccattaaatcttactgtgattaccacactgaaactcatcttgcagtttaatacagcaaa

SEQ ID NO: 331 - RNA, 747 nt, synthetic construct
  source 1..747: mol_type = other RNA
ttaaaacagctctggggttgttcccaccccagaggcccacgtggcggctagtacactggtattacggtacccttgtacgcctgttttatactcccttccccgtaacttagaagctatgtaatccaagttcaatagaagggggtacaaaccagtaccaccacgaacgagcacttctgtttccccggtgagattgtataagctgttcccacggccgaaaacgatcgatccgttacccgctcttgcacttcgagaagcctagtatcatcttgggatcttcgatgcgttgcgctcagcactctaccccagagtgtagcttaggtcgatgagtctggacattcctcaccggcgacggtggtccaggctgcgttggcggcctacctgtggtccaaagccacaggacgctagttgtaaacaaggtgtgaagagcctattgagctgcaagagagtcctccggcccctgaatgcggctaatcccaaccacggagcaggtggttgcaacccagtaactggcctgtcgtaacgcgtaagtctgtggcggaaccgactactttgggtgtccgtgtttccttttatttctataatggctgcttatggtgacaatcatagattgttatcataaagcgaattggattggccatccggtgagagttaagacaattatccacttgtttgttggatttacccctctgactaattacacactcaactttattaggattgtcattctagtgagatattaccatcaca

SEQ ID NO: 332 - RNA, 747 nt, synthetic construct
  source 1..747: mol_type = other RNA
ttaaaacagctctggggttgttcccaccccagaggcccacgtggcggctagtactccggtatcacggtacccttgtacgcctgttttacactcccttcccccgtaacttagaagaaacaaacaaagttcaatagaagggggtacaaaccagtaccaccacgaacaagcacttctgtttccccggtgacgttgtatagactgtacccacggtcgaaaacgattgatccgttatccgcttttgtacttcgagaagcctagtatcatcttggaatcttcgatgcgttgcgctcagcactcaatcccagagtgtagcttaggtcgatgagtctggacgttcctcaccggcgacggtggtccaggctgcgttggcggcctacctgtggcccaaagccacaggacgctagttgtgaacaaggtgtgaagagcctattgagctacaagagagtcctccggcccctgaatgcggctaatcctaaccacggagcaagggtgtgtgagccaacacataccttgtcgtaatgcgtaagttcgtggcggaaccgactactttgggtgtccgtgtttccttttattcttgtattggctgcttatggtgacaatcatagattgttatcataaagcgacttggattggccatccagtggaagtgaaataccttgtctacctgtctgttggttttactccatttgactaattcacttacaagcttattgctataatttttattgactaaacaacaatactaca

SEQ ID NO: 333 - RNA, 775 nt, synthetic construct
  source 1..775: mol_type = other RNA
caccctcttttccggcggcgcatgttcgcgtcgtcgtaagtctggattcccaaggcccactcggttcaacttcggtttccggacaaatacaaagaacctcagtcctcttggtactttctcgcctgagtctacaaagcgagaaacctgcccctctaacgccagacgagcggcataaaactcgaacttctggcacgttccaccaccccttctcctatcccaacccccattgcgctctcaaggtcgcgcttttccgagactagctcggattcaaaaagttcctggcaccctttaccccttcaggcccttaaggtaggaactgaccttgtgctgtgatctcggtgcggaagtgctactgcgtagtgattgtaagatccttttgtggttctgccctggcaaggctacagagtgctgtgatccgctgcggatgccatcctggtaacaggacccccagtgtgcgcaacagtatgttcacggtcttccgtgttcaccacattcggaacactgctttcgtgaaacagtgtgtgtccaatccctgtgatcagtatcaaccacaccacctaggaatgctaggaaggtaccccggttcgccgggatctgatcctaggctaattgtctacggtggtgctcctttttattttccacttcaactcactggttacaactgcttgatttctgtgtttgctgcttttctctgctctcactgccattctcaagtgttcacactgtccaagctcctttggttgttcgcttccacttgccactgtcaactcttgtc

SEQ ID NO: 334 - RNA, 740 nt, synthetic construct
  source 1..740: mol_type = other RNA
aacagcctgtgggttgtacccacccacagggcccactgggcgctagcacactggtatcacggtacctttgtgcgcctgttttataccccctaccccaactttaaacttagaagcaaagcaaacccgatcaatagcagatgtagcaagccagttgcatcttgatcaagtacttctgtctccccggactgagtatcaatagactgctcacgcggttgaaggagaaaacgttcgttacccgaccaactacttcgagaaacctagtaacaccatgaaagtagcagggtgtttcgatcagcacaaccccagtgtagatcaggctgatgagtcaccgcatcccccacgggcgaccgtggcggtggctgcgctggcggcctgcccatggggcaacccatgggacgctctaatatggacatggtgtgaagagtctattgagctagttggtagtcctccggcccctgaatgcggctaatcctaactgcggagcacataccccaaagccaaggggcagtgtgtcgtaacgggcaactctgcagcggaaccgactactttgggtgtccgtgtttccttttattctcaaactggctgcttatggtgacaatctcagagttgttaccatatagctattggattggccatccggtgagcaacagagcaattgtctaccaatttgttggatttattccactcaatctagcggttttcagaacattgaattatattctaaccctcaacaagacgaa

SEQ ID NO: 335 - RNA, 662 nt, synthetic construct
  source 1..662: mol_type = other RNA
tggaattaacatcatttccgacgaaagtgctatcatgcctccccgattgtgtgatgttttctgccctgctgggcggagcattctcgggttgagaaaccttgaatcttttcctttggaaccttggttcccccggtctaagccgcttggaatatgacagggttattttccttatcttacttctactttcacgggttctatccgtgaaaagggtacgtgttgccccttccttcttcggagaattcacacggcggtctttccgtctctcgacaggtgtgaatgcaacatgccggaaacggtgaagaaaacagttttctgcggaagtcttgagtgcgcatcgaaacagctgtagcgacctcacagtagcagcggactcccctcttggtgacaagagcctctgcggccaaaagccccgtggataagatccactgctgtgagcggtgcaaccccagcaccctgattcgatgaacattctttacggaaccagaagatggttttcttaagccctccggtagagaagccaagaatgtcctgaaggtaccccgcgtgcgggatctgatcaggagaccaattgactgtgctttacactgtcactttggtttaaaaactgtcacagcttctccaaaccaagtggtcttggttttccaattttactaactggcaat

SEQ ID NO: 336 - RNA, 665 nt, synthetic construct
  source 1..665: mol_type = other RNA
ttacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgaattccggtagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggc

SEQ ID NO: 337 - RNA, 739 nt, synthetic construct
  source 1..739: mol_type = other RNA
aacagcctgtgggttgtacccacccacagggcccactgggcgccagcactctggtattacggtacccttgtgcgcctgttttatatcccttccccaaggtaaacgttagaagcaatgcactcccgatcaatagaaggtgcgacgcgccagtcgtatctcgatcaagcacttctgtttccccggaccgagtatcaatagactgctagcgcggttgaaggagaaaacgttcgttacccgactaactacttcgagaagcccagtagcgccatgaaagttgcagagtgtttcgctcagcacttcccccgtgtagatcaggccgatgagtcaccgcaatccccacgggcgaccgtggcggtggctgcgttggcggcctgcctatggggaaacccataggacgctcaaatacagacatggtgcgaagagtctattgagctagttagtactcctccggcccctgaatgcggctaatcctaactgcggagcaagtactcacaatccagtgggcagcttgtcgtaacgggtaactctgcagcggaaccgactactttgggtgtccgtgtttccttttattcctacattggctgcttatggtgacaatcaaagagttgttaccatatagctattggattggccatccggtgactaataaagcaattatatttctttttgttgggtttgtaccccttaatacaaccagcttcaccacattgtgttacatcattagactgaacaaggcaaa

SEQ ID NO: 338 - RNA, 677 nt, synthetic construct
  source 1..677: mol_type = other RNA
tctttttgacgtggttggaattaacatcatttccgacgaaagtgctatcatgcctccccgattgtgtgatgttttctgccctgctgggcggagcattctcgggttgagaaaccttgaatcttttcctttggaaccttggttcccccggtctaagccgcttggaatatgacagggttattttccttatcttacttctactttcacgggttctatccgtgaaaagggtacgtgttgccccttccttcttcggagaattcacacggcggtctttccgtctctcgacaggtgtgaatgcaacatgccggaaacggtgaagaaaacagttttctgcggaagtcttgagtgcgcatcgaaacagctgtagcgacctcacagtagcagcggactcccctcttggtgacaagagcctctgcggccaaaagccccgtggataagatccactgctgtgagcggtgcaaccccagcaccctgattcgatgaacattctttacggaaccagaagatggttttcttaagccctccggtagagaagccaagaatgtcctgaaggtaccccgcgtgcgggatctgatcaggagaccaattgactgtgctttacactgtcactttggtttaaaaactgtcacagcttctccaaaccaagtggtcttggttttccaattttactaactggcaat

SEQ ID NO: 339 - RNA, 823 nt, synthetic construct
  source 1..823: mol_type = other RNA
tttgaaaagggggtgggggggcttcggccccctcaccctcttttccggcggcgcatgttcgcgtcgtcgtaagtctggattcccaaggcccactcggttcaacttcggtttccggacaaatacaaagaacctcagtcctcttggtactttctcgcctgagtctacaaagcgagaaacctgcccctctaacgccagacgagcggcataaaactcgaacttctggcacgttccaccaccccttctcctatcccaacccccattgcgctctcaaggtcgcgcttttccgagactagctcggattcaaaaagttcctggcaccctttaccccttcaggcccttaaggtaggaactgaccttgtgctgtgatctcggtgcggaagtgctactgcgtagtgattgtaagatccttttgtggttctgccctggcaaggctacagagtgctgtgatccgctgcggatgccatcctggtaacaggacccccagtgtgcgcaacagtatgttcacggtcttccgtgttcaccacattcggaacactgctttcgtgaaacagtgtgtgtccaatccctgtgatcagtatcaaccacaccacctaggaatgctaggaaggtaccccggttcgccgggatctgatcctaggctaattgtctacggtggtgctcctttttattttccacttcaactcactggttacaactgcttgatttctgtgtttgctgcttttctctgctctcactgccattctcaagtgttcacactgtccaagctcctttggttgttcgcttccacttgccactgtcaactcttgtcatggacccctcaaac

SEQ ID NO: 340 - RNA, 744 nt, synthetic construct
  source 1..744: mol_type = other RNA
ttaaaacagcctgtgggttgtacccacccacagggcccactgggcgctagcacactggtatcacggtacctttgtgcgcctgttttataccccctaccccaactttaaacttagaagcaaagcaaacccgatcaatagcagatgtagcaagccagttgcatcttgatcaagtacttctgtctccccggactgagtatcaatagactgctcacgcggttgaaggagaaaacgttcgttacccgaccaactacttcgagaaacctagtaacaccatgaaagtagcagggtgtttcgatcagcacaaccccagtgtagatcaggctgatgagtcaccgcatcccccacgggcgaccgtggcggtggctgcgctggcggcctgcccatggggcaacccatgggacgctctaatatggacatggtgtgaagagtctattgagctagttggtagtcctccggcccctgaatgcggctaatcctaactgcggagcacataccccaaagccaaggggcagtgtgtcgtaacgggcaactctgcagcggaaccgactactttgggtgtccgtgtttccttttattctcaaactggctgcttatggtgacaatctcagagttgttaccatatagctattggattggccatccggtgagcaacagagcaattgtctaccaatttgttggatttattccactcaatctagcggttttcagaacattgaattatattctaaccctcaacaagacgaa

SEQ ID NO: 341 - protein, 489 aa, synthetic construct; identical to SEQ ID NO: 173

SEQ ID NO: 342 - protein, 485 aa, synthetic construct; identical to SEQ ID NO: 59

SEQ ID NO: 343 - protein, 9 aa, synthetic construct
GGGGSCAAA

SEQ ID NO: 344 - protein, 10 aa, synthetic construct
GGGGSGGGGS

SEQ ID NO: 345 - protein, 13 aa, synthetic construct
GGGGSGGGGSCAS

SEQ ID NO: 346 - protein, 701 aa, synthetic construct
GCCAAGGAACCTCAGTCACCGTCTCCTCAACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACAAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAA

SEQ ID NO: 347 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 348 - RNA, 93 nt, synthetic construct
  source 1..93: mol_type = other RNA
tcggcgaccatttgtgtggtgagctctaagtgttgatcagcgggcgcagctgcatatgacctctgcgtgtgcctggtaattgactaagatatc

SEQ ID NO: 349 - RNA, 76 nt, synthetic construct
  source 1..76: mol_type = other RNA
taatagaacaaacacaaaccaaacaccattgtcacactccaaacaacaaacaaacaaccacacaaatggtcgccga

SEQ ID NO: 350 - RNA, 73 nt, synthetic construct
  source 1..73: mol_type = other RNA
taatagaaaaacacaaaccaaacaccattgtcacactccaaaaaaaacaaacaaccacacaaatggtcgccga

SEQ ID NO: 351 - RNA, 2332 nt, synthetic construct; identical to SEQ ID NO: 264
  source 1..2332: mol_type = other RNA

SEQ ID NO: 352 - RNA, 108 nt, synthetic construct; identical to SEQ ID NO: 43
  source 1..108: mol_type = other RNA

SEQ ID NO: 353 - RNA, 93 nt, synthetic construct; identical to SEQ ID NO: 348
  source 1..93: mol_type = other RNA

SEQ ID NO: 354 - RNA, 675 nt, synthetic construct; identical to SEQ ID NO: 1
  source 1..675: mol_type = other RNA

SEQ ID NO: 355 - RNA, 1458 nt, synthetic construct; identical to SEQ ID NO: 266
  source 1..1458: mol_type = other RNA

SEQ ID NO: 356 - RNA, 22 nt, synthetic construct
  source 1..22: mol_type = other RNA
caaacaccattgtcacactcca

SEQ ID NO: 357 - RNA, 50 nt, synthetic construct; identical to SEQ ID NO: 267
  source 1..50: mol_type = other RNA

SEQ ID NO: 358 - RNA, 91 nt, synthetic construct; identical to SEQ ID NO: 268
  source 1..91: mol_type = other RNA

SEQ ID NO: 359 - RNA, 76 nt, synthetic construct; identical to SEQ ID NO: 349
  source 1..76: mol_type = other RNA

SEQ ID NO: 360 - RNA, 2329 nt, synthetic construct; identical to SEQ ID NO: 228
  source 1..2329: mol_type = other RNA

SEQ ID NO: 361 - RNA, 47 nt, synthetic construct; identical to SEQ ID NO: 270
  source 1..47: mol_type = other RNA

SEQ ID NO: 362 - RNA, 88 nt, synthetic construct; identical to SEQ ID NO: 46
  source 1..88: mol_type = other RNA

SEQ ID NO: 363 - RNA, 73 nt, synthetic construct; identical to SEQ ID NO: 350
  source 1..73: mol_type = other RNA

SEQ ID NO: 364 - RNA, 2332 nt, synthetic construct
  source 1..2332: mol_type = other RNA
tcggcgaccatttgtgtggtgagctctaagtgttgatcagcgggcgcagctgcatatgacctctgcgtgtgcctggtaattgactaagatatcttactcagcgtaactactccgggttacgtgatgaagaagaggctacggagattctcgggctacggccctggagccactccggctcctaaagatttagaagtttgagcacacccgcccactagggccccccatccaggggggcaacgggcaagcacttctgtttccccggtatgatctgataggctgtaaccacggctgaaacagagattatcgttatccgcttcactacttcgagaagcctagtaatgatgggtgaaattgaatccgttgatccggtgtctcccccacaccagaaactcatgatgagggttgccatcccggctacggcgacgtagcgggcatccctgcgctggcatgaggcctcttaggaggacggatgatatggatcttgtcgtgaagagcctattgagctagtgtcgactcctccgcccccgtgaatgcggctaatcctaaccccggagcaggtgggtccaatccagggcctggcctgtcgtaatgcgtaagtctgggacggaaccgactactttcgggaaggcgtgtttccatttgttcattatttgtgtgtttatggtgacaactctgggtaaacgttctattgcgtttattgagagattcccaacaattgaacaaacgagaactacctgttttattaaatttacacagagaagaattacaatggctctgcctgtgacagctctgctgctgcctctggctctgcttctgcatgccgccagacctgacatccagatgacccagacaaccagcagcctgtctgccagcctgggcgacagagtgaccatcagctgtagagccagccaggacatcagcaagtacctgaactggtatcagcagaaacccgacggcaccgtgaagctgctgatctaccacaccagcagactgcacagcggcgtgccatccagattttctggcagcggctctggcaccgactacagcctgacaatcagcaacctggaacaagaggatatcgctacctacttctgccagcaaggcaacaccctgccttacacattcggcggaggcaccaagctggaaatcacaggtggcggaggatctggcggaggtggaagcggaggcggtggatctgaagtgaaactgcaagagtctggccctggcctggtggctccatctcagtctctgagcgtgacctgtaccgtcagcggagtgtctctgcctgattacggcgtgtcctggatcagacagcctcctagaaaaggcctggaatggctgggcgtgatctggggcagcgagacaacctactacaacagcgccctgaagtccaggctgaccatcatcaaggacaactccaagagccaggtgttcctgaagatgaacagcctgcagaccgacgacaccgccatctactactgcgccaagcactactactacggcggcagctacgccatggattactggggccagggaaccagcgtgaccgtgtctagcacaacaacccctgctcctagacctcctacaccagctcctacaatcgccagccagcctctgtctctgaggccagaggcttgtagacctgctgctggcggagctgtgcacacaagaggcctggatttcgcctgcgacatctacatctgggcccctctggctggaacatgtggcgtgctgctgctgagcctggtcatcaccctgtattgcaagagaggccggaagaagctgctctacatcttcaagcagcccttcatgaggcccgtgcagaccacacaagaagaggacggctgctcctgcagattccccgaggaagaggaaggcggctgcgagctgagagtgaagttcagcagaagcgctgacgcccctgcttaccagcagggacagaaccagctgtacaacgagctgaacctggggagaagagaagagtacgacgtgctggacaagcggagaggcagagatcctgagatgggcggcaagcccagaagaaagaaccctcaagagggcctgtataatgagctgcagaaagacaagatggccgaggcctacagcgagatcggaatgaagggcgagcgcagaagaggcaagggacacgatggactgtaccagggcctgagcacagccaccaaggacacatatgacgccctgcacatgcaggccctgcctccaagataatagaacaaacacaaaccaaacaccattgtcacactccaaacaacaaacaaacaaccacacaaatggtcgccgaaactactgaaagcataacttttagttattt

SEQ ID NO: 365 - DNA, 38 bp, synthetic construct
  source 1..38: mol_type = other DNA
ggccagtgaattgtaatacgactcactatagggagaat

SEQ ID NO: 366 - RNA, 25 nt, synthetic construct
  source 1..25: mol_type = other RNA
aaaaaaaaaaaaaaaaaaaaaaaaa

SEQ ID NO: 367 - DNA, 16 bp, synthetic construct
  source 1..16: mol_type = other DNA
tcaccgaccgaccggt

SEQ ID NO: 368 - DNA, 149 bp, synthetic construct
  source 1..149: mol_type = other DNA
aataagagaaagtgcaacgactattccgataggaagtagggtcaagtgactcgaaatggggattacccttctagggtagtgatatagtctgaacatatatggaaacatatagaaggataggagtaacgaacctattcgtaacataattg

SEQ ID NO: 369 - DNA, 15 bp, synthetic construct
  source 1..15: mol_type = other DNA
aacttttagttattt

SEQ ID NO: 370 - DNA, 20 bp, synthetic construct
  source 1..20: mol_type = other DNA
tcggcgaccatttgtgtggt

SEQ ID NO: 371 - DNA, 50 bp, synthetic construct
  source 1..50: mol_type = other DNA
taagtgttgatcagcgggcgcagctgcatatgacctctgcgtgtgcctgg

SEQ ID NO: 372 - DNA, 11 bp, synthetic construct
  source 1..11: mol_type = other DNA
taattgactaa

SEQ ID NO: 373 - DNA, 13 bp, synthetic construct
  source 1..13: mol_type = other DNA
aacaaacacaaac

SEQ ID NO: 374 - DNA, 15 bp, synthetic construct
  source 1..15: mol_type = other DNA
aacaacaaacaaaca

SEQ ID NO: 375 - DNA, 20 bp, synthetic construct
  source 1..20: mol_type = other DNA
accacacaaatggtcgccga

SEQ ID NO: 376 - DNA, 15 bp, synthetic construct
  source 1..15: mol_type = other DNA
aactactgaaagcat

SEQ ID NO: 377 - DNA, 119 bp, synthetic construct
  source 1..119: mol_type = other DNA
aaataattgtgcctttatacagtaatgtatatcgaaaaatcctctaattcagggaacacctaaacaaactaagatgtaggcaatcctgagctaagctcttagtacgcgttcagatagat

SEQ ID NO: 378 - DNA, 10 bp, synthetic construct
  source 1..10: mol_type = other DNA
atctagaacg

SEQ ID NO: 379 - DNA, 48 bp, synthetic construct
  source 1..48: mol_type = other DNA
ctagcataaccccttggggcctctaaacgggtcttgaggggttttttg

SEQ ID NO: 380 - RNA, 16 nt, synthetic construct
  source 1..16: mol_type = other RNA
tcaccgaccgaccggt

SEQ ID NO: 381 - RNA, 149 nt, synthetic construct
  source 1..149: mol_type = other RNA
aataagagaaagtgcaacgactattccgataggaagtagggtcaagtgactcgaaatggggattacccttctagggtagtgatatagtctgaacatatatggaaacatatagaaggataggagtaacgaacctattcgtaacataattg

SEQ ID NO: 382 - RNA, 15 nt, synthetic construct
  source 1..15: mol_type = other RNA
aacttttagttattt

SEQ ID NO: 383 - RNA, 20 nt, synthetic construct
  source 1..20: mol_type = other RNA
tcggcgaccatttgtgtggt

SEQ ID NO: 384 - RNA, 50 nt, synthetic construct
  source 1..50: mol_type = other RNA
taagtgttgatcagcgggcgcagctgcatatgacctctgcgtgtgcctgg

SEQ ID NO: 385 - RNA, 11 nt, synthetic construct
  source 1..11: mol_type = other RNA
taattgactaa

SEQ ID NO: 386 - RNA, 13 nt, synthetic construct
  source 1..13: mol_type = other RNA
aacaaacacaaac

SEQ ID NO: 387 - RNA, 15 nt, synthetic construct
  source 1..15: mol_type = other RNA
aacaacaaacaaaca

SEQ ID NO: 388 - RNA, 20 nt, synthetic construct
  source 1..20: mol_type = other RNA
accacacaaatggtcgccga

SEQ ID NO: 389 - RNA, 15 nt, synthetic construct
  source 1..15: mol_type = other RNA
aactactgaaagcat

SEQ ID NO: 390 - RNA, 119 nt, synthetic construct
  source 1..119: mol_type = other RNA
aaataattgtgcctttatacagtaatgtatatcgaaaaatcctctaattcagggaacacctaaacaaactaagatgtaggcaatcctgagctaagctcttagtacgcgttcagatagat

SEQ ID NO: 391 - RNA, 12 nt, synthetic construct
  source 1..12: mol_type = other RNA
aaaaacacaaac

SEQ ID NO: 392 - RNA, 13 nt, synthetic construct
  source 1..13: mol_type = other RNA
aaaaaaacaaaca

SEQ ID NO: 393 - RNA, 15 nt, synthetic construct
  source 1..15: mol_type = other RNA
aaaaaaaaaaaaaaa

SEQ ID NO: 394 - RNA, 15 nt, synthetic construct
  source 1..15: mol_type = other RNA
ccccccccccccccc

SEQ ID NO: 395 - RNA, 16 nt, synthetic construct
  source 1..16: mol_type = other RNA
cacacacacacacaca

SEQ ID NO: 396 - RNA, 57 nt, synthetic construct
  source 1..57: mol_type = other RNA
taaaccggactgttaccaacacccacacccctgtgatgaaacaaaacccataaatgc

SEQ ID NO: 397 - RNA, 39 nt, synthetic construct
  source 1..39: mol_type = other RNA
aagttctacaatgcatctgggactttgattgtacattgt

SEQ ID NO: 398 - RNA, 31 nt, synthetic construct
  source 1..31: mol_type = other RNA
taaaccggactgtaaaaaaacccataaatgc

SEQ ID NO: 399 - RNA, 109 nt, synthetic construct
  source 1..109: mol_type = other RNA
ggcggcgtcagggtcgcagcgtctacagctgctcgggggcggtttcttggcggaggcttggccggctcctctctcccggctccgcggcggctgcgaaggcggcggctcc

SEQ ID NO: 400 - RNA, 138 nt, synthetic construct
  source 1..138: mol_type = other RNA
cggcgatcccggcggagggggccgttcgccagctctgaggcagaaagtgccacgactccacacgcgcgccggcgatcccggcggagggggccgttcgccagctctgaggcagaaagtgccacgactccacacgcgcgc

SEQ ID NO: 401 - RNA, 41 nt, synthetic construct
  source 1..41: mol_type = other RNA
acgcagccagcgagcggccggagcggacggcagacggggcg

SEQ ID NO: 402 - RNA, 58 nt, synthetic construct
  source 1..58: mol_type = other RNA
tgccctctcgctttccctctcgcgtctctggctgcaggtgaaaggaaagcaagccagg

SEQ ID NO: 403 - RNA, 27 nt, synthetic construct
  source 1..27: mol_type = other RNA
agaccgtgcaccatgagcacgaatcca

SEQ ID NO: 404 - RNA, 22 nt, synthetic construct
  source 1..22: mol_type = other RNA
cacctatatagttatataataa

SEQ ID NO: 405 - RNA, 30 nt, synthetic construct
  source 1..30: mol_type = other RNA
caagtggacagcaattcacactgtctctgg

SEQ ID NO: 406 - RNA, 40 nt, synthetic construct
  source 1..40: mol_type = other RNA
caagtggacagcaattcacactgtctctgtcacctgcagg

SEQ ID NO: 407 - RNA, 43 nt, synthetic construct
  source 1..43: mol_type = other RNA
tgtataataaaatgccgaatgtctctcgatctgatcaaaccag

SEQ ID NO: 408 - RNA, 42 nt, synthetic construct
  source 1..42: mol_type = other RNA
gagtgccagaagctgcctcttcggaggcagtttctggtactc

SEQ ID NO: 409 - RNA, 34 nt, synthetic construct
  source 1..34: mol_type = other RNA
ggcagaagctgcctcttcggaggcagtttctgcc

SEQ ID NO: 410 - RNA, 40 nt, synthetic construct
  source 1..40: mol_type = other RNA
tgaaaaattttaaaaagcgatgagataaaaagttaatttg

SEQ ID NO: 411 - RNA, 75 nt, synthetic construct
  source 1..75: mol_type = other RNA
ctcctgtttcattatttgcgtgaggcattcttgtatctcttgcaaagttttcttttttctttcactttcgcactg

SEQ ID NO: 412 - RNA, 75 nt, synthetic construct
  source 1..75: mol_type = other RNA
agactctgtgtctgtgtgtgcattagtatttcgtcttattcctttccttttcttgctgtaccattgtatttattc

SEQ ID NO: 413 - RNA, 75 nt, synthetic construct
  source 1..75: mol_type = other RNA
aaggccagctcagccatatactcttaccacatactcaaattctgctgctctgctctgcttttgcgttgctgagtt

SEQ ID NO: 414 - RNA, 75 nt, synthetic construct
  source 1..75: mol_type = other RNA
tcttgtttgtcttctaaacactactataaagcgtctgcgtttttctcttcgtgtgtatttctcttttttttcgca

SEQ ID NO: 415 - RNA, 22 nt, synthetic construct
  source 1..22: mol_type = other RNA
gcataaaaagcataaaaagcat

SEQ ID NO: 416 - RNA, 79 nt, synthetic construct
  source 1..79: mol_type = other RNA
aagactatgttaaaatacgcgaagaagtggctcatttcaagccattaagtatacccaacttaactaataatcaaaataa

SEQ ID NO: 417 - DNA, 17 bp, synthetic construct
  source 1..17: mol_type = other DNA
taatacgactcactata

SEQ ID NO: 418 - DNA, 13 bp, synthetic construct
  source 1..13: mol_type = other DNA
ggccagtgaattg

(19) United States
(12) Patent Application Publication
(10) Pub. No.: US 2026/0250349 A1
(43) Pub. Date: Aug. 27, 2026

(54) CIRCULAR RNA COMPOSITIONS AND METHODS OF USE THEREOF FOR TARGETED DELIVERY

(71) Applicant: Orbital Therapeutics, Inc., Cambridge, MA (US)

(72) Inventors: Gilles Besin, Chestnut Hill, MA (US); Robert Jeffrey Blakemore, Stoneham, MA (US); Benjamin Mahler Geilich, Arlington, MA (US); Nahal Habibi, Cambridge, MA (US); Kristen Ott, Medfield, MA (US); Emrah Ilker Ozay, Watertown, MA (US); Trent Parker Stevens, Wakefield, MA (US)

(21) Appl. No.: 19/549,096

(22) Filed: Feb. 25, 2026

Related U.S. Application Data
(60) Provisional application No. 63/763,317, filed on Feb. 26, 2025.
(60) Provisional application No. 63/846,699, filed on Jul. 18, 2025.
(60) Provisional application No. 63/929,029, filed on Dec. 2, 2025.

(51) Int. Cl. C07K 14/725

(52) U.S. Cl. CPC C07K 14/7051; C07K 2317/24; C07K 2317/55; C07K 2317/56; C07K 2317/76; C07K 2317/92

(57) ABSTRACT

The present disclosure provides compositions, methods of making, and use of a circular ribonucleic acid (circRNA) therapeutic for targeted delivery to immune cells, including T cells, for effector functions such as B cell depletion, providing, among other things, methods of treating B cell-mediated diseases such as autoimmune disease and cancer, meeting the medical need for efficacious and safe therapies that overcome the disadvantages of ex vivo CAR T therapy. Provided by the present disclosure is a circRNA therapeutic comprising: (i) a circRNA comprising a ribosome recruiting element, and a coding sequence encoding a CAR comprising, for example, an anti-CD19 antibody or antigen-binding fragment thereof, and (ii) a targeting moiety (e.g., anti-CD5) that binds to an immune cell (e.g., a T cell), wherein the circRNA is in a delivery vehicle, and a targeting moiety that binds to a T cell is conjugated to the delivery vehicle (e.g., a lipid nanoparticle).

### CROSS REFERENCE TO RELATED APPLICATIONS

The present application claims priority to, and the benefit of U.S. Provisional Application No. 63/763,317, filed on Feb. 26, 2025, U.S. Provisional Application No. 63/846,699, filed on Jul. 18, 2025, and U.S. Provisional Application No. 63/929,029, filed on Dec. 2, 2025, the contents of which are incorporated herein by reference in their entireties.

### INCORPORATION BY REFERENCE OF SEQUENCE LISTING

The instant application contains a Sequence Listing which has been submitted electronically in XML format and is hereby incorporated by reference in its entirety. The sequence listing file, created on Feb. 20, 2026, is named ORB-019WO1.xml and is 630,784 bytes in size.

### BACKGROUND

In various diseases such as autoimmune disease and cancer, B cells contribute to disease pathogenesis via local production of inflammatory cytokines or by acting as antigen presenting cells (Sanz, 2010, Sarvaria et al., 2017). Autoantibodies produced by B cells are pathogenic in certain disease types (Ramirez-Valle, 2024). Most patients with autoimmune diseases fail to eliminate autoreactive B cells and autoantibodies due to impaired B cell tolerance (Pillai, 2011). Some clinical studies have shown that depletion of B cells has been associated with positive patient outcomes (Arnold, 2024; English, 2024; Blache, 2023).

Ex vivo chimeric antigen receptor (CAR) T cell therapy has several disadvantages, such as manufacturing processes that are complex, laborious, and expensive. Ex vivo CAR therapy requires patients' or donors' T cells to be isolated from peripheral blood via apheresis, activated, and subsequently transduced by a retrovirus (e.g., lentivirus or gammaretrovirus) comprising a chimeric antigen receptor (CAR) construct to create an engineered CAR T cell. CAR T cells are further expanded before infusing the engineered T cells into a patient's bloodstream to attack target cells. Further, ex vivo CAR therapy requires lymphodepletion before administration, and is also associated with serious side effects, including cytokine release syndrome (CRS), which can cause multi-organ failure and even death, and immune effector cell-associated neurotoxicity syndrome (ICANS), which is a neuropsychiatric syndrome that can cause confusion and seizures (Bui, 2024; Mullard, 2024).

There is an unmet need for efficacious and safe therapies to treat diseases such as autoimmune disease and cancer that overcome the disadvantages of ex vivo CAR therapy.

### SUMMARY OF THE INVENTION

The present disclosure provides, among other things, an improved in vivo CAR therapy for treating B cell-mediated diseases based on circular ribonucleic acids (circRNA). The present disclosure provides, among other things, targeted in vivo delivery of therapeutic genes to select cell types (e.g., immune cells, or, more specifically, T cells) for treating B cell-mediated diseases (e.g., autoimmune disease or cancer). The present disclosure provides, in part, a circular ribonucleic acid for targeted delivery comprising: (i) a circRNA comprising a coding sequence encoding a chimeric antigen receptor (CAR) comprising an anti-CD19 antibody or antigen-binding fragment thereof, (ii) a targeting moiety, and (iii) a delivery vehicle, wherein the targeting moiety is conjugated to the delivery vehicle (e.g., lipid nanoparticle, viral particle, among others).

CircRNA therapeutics provide multiple advantages compared to linear RNA. For example, circRNA therapeutics provides enhanced stability. Without wishing to be bound by any mechanism, since circRNA is a closed molecule with no free ends, it confers resistance to exonucleases, which leads to RNA durability. In addition, the absence of open 5′ and 3′ ends leads to potential reduction of an innate immune response by the patient. Moreover, circular RNA allows for efficient translation compared to linear RNA; IRES-mediated translation (cap-independent) leads to high synthesis of the protein encoded by the circular RNA.

The present disclosure provides, in part, a circular ribonucleic acid for targeted delivery comprising: (i) a circRNA comprising a ribosome recruiting element (e.g., an Internal Ribosome Entry Site (IRES), IRES transacting factor (ITAF) recruiting element, polypyrimidine tract binding protein (PTB), branched cap or qRNA, etc.), and a coding sequence encoding a chimeric antigen receptor (CAR) comprising an anti-CD19 antibody or antigen-binding fragment thereof, (ii) a targeting moiety, wherein the targeting moiety binds to a T cell, wherein the circRNA is in a delivery vehicle, and a targeting moiety that binds to a T cell is conjugated to the delivery vehicle (e.g., a nanoparticle, including for example, lipid nanoparticle, viral particle, among others).

Exemplary targeting moieties of the present disclosure for targeting immune cells, more specifically, T cells, include, without limitation, anti-CD2, anti-CD3, anti-CD5, anti-CD7, or anti-CD8 antibody or antigen-binding fragment thereof (e.g., more specifically, anti-CD5 antibody or antigen-binding fragment thereof). The present disclosure thus provides for specific targeting of a circRNA that comprises a chimeric antigen receptor comprising an anti-CD19 antibody or antigen-binding domain to T cells, thus localizing the anti-CD19 CAR to a specific cell type required for its action and minimizing off-target effects, thereby increasing safety and efficacy. In some embodiments, safety is further enhanced by the inclusion of microRNA binding sites in the untranslated regions (UTR) of the circRNA to minimize toxicity due to off-target effects.

In some embodiments, a single microRNA binding site (e.g., miR-122) is included in the 3′ UTR. In some embodiments, the microRNA binding site is flanked by an upstream spacer and a downstream spacer. Without wishing to be bound by any particular theory, the upstream and the downstream spacer facilitate circularization of the precursor RNA into circular RNA. In some embodiments, the upstream spacer and the downstream spacer are designed to have poly A sequences that are interrupted by insertion of a cytosine. In some embodiments, the upstream spacer and the downstream spacer are designed to have a hydropathy index that favors limiting interaction of the encapsulated circRNA with the lipid nanoparticle, facilitating release into a target cell.

In some embodiments, the circRNAs and circRNA therapeutics of the present disclosure were precisely engineered for in vivo CAR therapy. In the present disclosure, the circRNAs and circRNA therapeutics stably and durably express CAR to high levels, in part, due to the increased stability of circRNA relative to a linear RNA; they are codon-optimized for high expression of a CAR construct, for example, anti-CD19 CAR construct; include variant 5′ and/or 3′ untranslated regions; and ribosome recruiting elements, for example, cell type-optimized Internal Ribosome Entry Site (IRES) elements that drive unexpectedly high expression of the CAR construct, including anti-CD19 CAR coding sequence, in select cell types, for example, T cells.

Expression of anti-CD19 CAR by T cells causes activation of T cells and specific binding to target B cells to carry out B cell depletion. Such a B cell compartment immune reset depletes the existing population of B cells, allowing a new generation of naïve B cell development from the bone marrow, thereby resetting the B cell immune population by, for example, depleting autoreactive B cells in autoimmune diseases and malignant B cells in B cell-mediated cancers, thereby treating the B cell-mediated disease.

In some embodiments of the present disclosure, a targeting moiety is conjugated to a delivery vehicle (e.g., lipid nanoparticles or viral particles) by various exemplary conjugations, including as non-limiting examples, chemical conjugations (thiol-maleimide, azide-dibenzocyclooctyne (DBCO), transcyclooctene (TCO)-tetrazine), and enzymatic conjugations (such as transglutaminase-catalyzed glutamine-amine conjugation, and sortase-catalyzed conjugation (such as a sortase-catalyzed LPXT/(G)n-(G)n conjugation, wherein, for e.g., n=1-5 and X is any amino acid residue), among other conjugation strategies. Exemplary conjugation strategies are described in FIG. 13A and exemplary conjugation handles are provided in Table 12.

LNP delivery, among other things, increases stability, bioavailability, and distribution of circRNA to target tissues. The LNP formulations disclosed herein ensure that the circRNA encapsulated by the LNP are protected from degradation during delivery, and allow for efficient and targeted delivery to target cells, e.g., T cells. Provided herein are efficient methods of making stable lipid nanoparticles with sufficient LNP properties, such as size, polydispersity and efficacy in encapsulating circRNA (as measured by percent encapsulation) for therapeutic applications. The conjugation handles and conjugation methods disclosed herein for conjugating the lipid nanoparticle to a targeting moiety minimizes toxicity from the delivery and accumulation of circRNA at unintended, off-target cells, and facilitates specific targeting.

In some embodiments, lipid nanoparticles of the present disclosure contain multiple polyethylene glycol (PEG)-lipids. In some embodiments, lipid nanoparticles of the present disclosure contain two or more PEG-lipids. In some embodiments, lipid nanoparticles of the present disclosure contain two PEG-lipids, wherein one PEG is used for conjugation and the other PEG is structural. Although PEG was used for conjugation, increasing the total PEG used for conjugation did not increase targeting. Counterintuitively, a lower total PEG content (e.g., 0.1% PEG) was associated with higher CAR expression.

Further, in some embodiments, the conjugating PEG comprises a long chain lipid anchor component between about C14 and C22 (e.g., C18 PEG). As described in more detail in the Examples, in some embodiments, a higher mole percent of total C18 PEG (conjugating PEG and structural PEG) was found to decrease LNP distribution to the liver (off-target organ) and increase LNP distribution to the spleen (target organ).

In further embodiments, ionizable lipids and the ratios of different lipid components were optimized to achieve specific targeting to T cells. In some embodiments, increasing phospholipid mole percentage (mol %), for example, increasing distearoylphosphatidylcholine (DSPC) mole percentage (mol %), increased potency and decreased off-target expression in liver.

In summary, the present disclosure provides, in part, a highly stable, durable and targeted in vivo CAR therapy encoded by circRNA that is achieved through an unexpected and surprising synergy of a combination of precisely engineered features in the circRNA, delivery vehicle (e.g., lipid nanoparticle), and targeting moiety conjugated to the delivery vehicle. In some embodiments, the targeted in vivo CAR therapy in the present disclosure provides for in vivo expression of the CAR therapy in specific cells, and use of circRNA improves the duration and magnitude of CAR expression on T cells. As described herein, the targeted in vivo CAR therapy reaches the specific cell types and tissues, e.g., T cells, and allows for enhanced in vivo CAR T generation and improves potency for B cell depletion. Among other things, the targeted in vivo CAR T therapy of the present disclosure requires no apheresis or lymphodepletion and is associated with greater ease of manufacturing and lower costs. The patient's own body serves as a manufacturer of CAR-T cells, and this in vivo CAR therapy may be less prone to cytokine release syndrome and toxicity effects.

In some aspects, provided herein is a circular ribonucleic acid (circRNA) therapeutic for targeted delivery comprising: (i) a circRNA comprising a ribosome recruiting element, and a coding sequence encoding a chimeric antigen receptor (CAR) comprising an anti-CD19 antibody or antigen-binding fragment thereof, and (ii) a targeting moiety, wherein the circRNA is encapsulated in a delivery vehicle, wherein the targeting moiety is conjugated to the delivery vehicle, and wherein the targeting moiety comprises an anti-CD5 antibody heavy chain variable region comprising CDR1, CDR2, and CDR3 sequences from SEQ ID NO: 89.

In some embodiments, the CDR1 sequence comprises SEQ ID NO: 64, the CDR2 sequence comprises SEQ ID NO: 65, and the CDR3 sequence comprises SEQ ID NO: 66.

In some aspects, provided herein is a circular ribonucleic acid (circRNA) for targeted delivery comprising: (i) a circRNA comprising: a ribosome recruiting element, and a coding sequence encoding a chimeric antigen receptor (CAR) comprising an anti-CD19 antibody or antigen-binding fragment thereof, and (ii) a targeting moiety, wherein the targeting moiety binds to a T cell, wherein the circRNA is in a delivery vehicle, wherein the targeting moiety that binds to a T cell is conjugated to the delivery vehicle, and wherein the targeting moiety is an anti-CD5 antibody or antigen-binding fragment thereof comprising a heavy chain variable region comprising complementarity determining region (CDR) sequences GFTFSMYS (SEQ ID NO: 64), ISTGARDT (SEQ ID NO: 65) and GDLRYGPDGYDY (SEQ ID NO: 66).

In some embodiments, the delivery vehicle is a lipid nanoparticle (LNP).

In some embodiments, the anti-CD5 antibody or antigen-binding fragment comprises a sequence having at least 95% identity to SEQ ID NO: 89.

In some embodiments, the anti-CD5 antibody or antigen-binding fragment comprises SEQ ID NO: 89.

In some embodiments, the anti-CD5 antibody or antigen-binding fragment is a VHH.

In some embodiments, the anti-CD5 antibody or antigen-binding fragment is conjugated to a conjugating PEG-lipid in the delivery vehicle.

In some embodiments, the anti-CD5 antibody or antigen-binding fragment is covalently attached to a conjugation handle.

In some embodiments, the conjugation handle is covalently attached to the C-terminus of the heavy chain of the anti-CD5 antibody or antigen-binding fragment.

In some embodiments, the conjugation handle is 5-10 amino acids in length.

In some embodiments, the conjugation handle comprises SEQ ID NO: 120.

In some embodiments, the conjugation handle consists of SEQ ID NO: 120.

In some embodiments, the anti-CD5 antibody or antigen-binding fragment is covalently attached to the conjugation handle, and the sequence of the anti-CD5 antibody or antigen-binding fragment with attached conjugation handle comprises SEQ ID NO: 122.

In some embodiments, the anti-CD5 antibody or antigen-binding fragment is conjugated to the conjugating PEG-lipid in the delivery vehicle via a thiol-maleimide conjugation.

In some embodiments, the anti-CD5 antibody or antigen-binding fragment is conjugated to the conjugating PEG-lipid in the delivery vehicle via a thiol-maleimide conjugation between the conjugating PEG-lipid and the conjugation handle.

In some embodiments, the delivery vehicle is an LNP comprising two PEG-lipids, one of which is the conjugating PEG-lipid.

In some embodiments, the conjugating PEG-lipid comprises a long chain fatty acid.

In some embodiments, the conjugating PEG-lipid is DSPE-PEG 2000-maleimide.

In some embodiments, the LNP further comprises one or more ionizable lipids, one or more phospholipids, and one or more sterol lipids.

In some embodiments, the LNP comprises the one or more ionizable lipids at 30 to 49 mol % (e.g., at 30 to 45 mol %), the one or more phospholipids at 5 to 40 mol % (e.g., at 15 to 35 mol %), the one or more sterol lipids at 15 to 50 mol % (e.g., at 30 to 50 mol %), the PEG lipid (e.g., the DMG-PEG 2000) at 0 to 10 mol % (e.g., at 0 to 5 mol %), and the conjugating PEG-lipid (e.g., the DSPE-PEG 2000-maleimide) at 0.01-1 mol %.

In some embodiments, the LNP comprises the one or more ionizable lipids at 30 to 49 mol %, the one or more phospholipids at 5 to 40 mol %, the one or more sterol lipids at 15 to 50 mol %, the DMG-PEG 2000 lipid at 0 to 10 mol %, and the DSPE-PEG 2000-maleimide lipid at 0.01-1 mol %.

In some embodiments, the LNP comprises the one or more ionizable lipids at 30 to 45 mol %, the one or more phospholipids at 15 to 35 mol %, the one or more sterol lipids at 15 to 50 mol %, the DMG-PEG 2000 lipid at 0 to 5 mol %, and the DSPE-PEG 2000-maleimide lipid at 0.01-1 mol %.

In some embodiments, the LNP comprises the one or more ionizable lipids, the one or more phospholipids, the one or more sterol lipids, the DMG-PEG 2000 lipid, and the DSPE-PEG 2000-maleimide lipid at a ratio of 39:20:39:1.9:0.1 mol %.

In some embodiments, the LNP comprises the one or more ionizable lipids, the one or more phospholipids, the one or more sterol lipids, the DMG-PEG 2000 lipid, and the DSPE-PEG 2000-maleimide lipid combined at a molar ratio of 39:20:39:1.9:0.1.

In some embodiments, the one or more ionizable lipids is

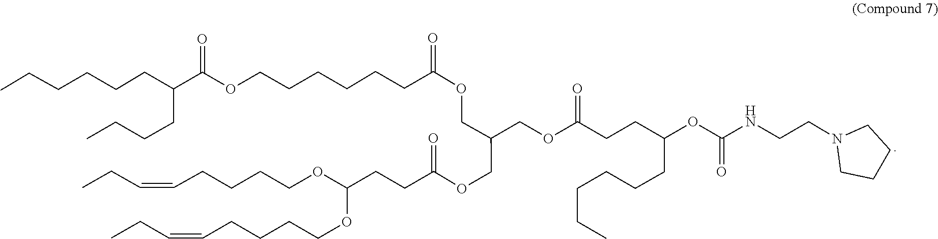 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN1CCCC1)OCCCC/C=C\CC]

In some embodiments, the one or more phospholipids is 1,2-distearoyl-snglycero-3-phosphocholine (DSPC).

In some embodiments, the sterol lipid is cholesterol.

In some embodiments, the ribosome recruiting element is an Internal Ribosome Entry Site (IRES), wherein the IRES comprises a sequence selected from Table 1.

In some embodiments, the IRES comprises a sequence selected from SEQ ID NO: 1-11 of Table 1.

In some embodiments, the IRES comprises SEQ ID NO: 1.

In some embodiments, the coding sequence further encodes a CD8 signal peptide, and the CAR comprises a CD8 hinge, a CD8 transmembrane domain, a 4-1BB costimulatory domain, and a CD3 zeta signaling domain.

In some embodiments, the anti-CD19 antibody or antigen-binding fragment thereof comprises a VL sequence of SEQ ID NO: 54 and a VH sequence of SEQ ID NO: 55.

In some embodiments, the coding sequence encodes an amino acid sequence of SEQ ID NO: 58.

In some aspects, provided herein is a circular ribonucleic acid (circRNA) therapeutic for targeted delivery comprising: a circular RNA comprising a ribosome recruiting element and a coding sequence encoding a chimeric antigen receptor (CAR) comprising an anti-CD19 antibody or antigen-binding fragment thereof, wherein the circRNA is encapsulated in an LNP, wherein a targeting moiety is conjugated to the LNP, wherein the targeting moiety is an anti-CD5 VHH, the anti-CD5 VHH comprising CDR1, CDR2, and CDR3 sequences from SEQ ID NO: 89, wherein the LNP comprises one or more ionizable lipids at 30 to 49 mol % (e.g, at 30 to 45 mol %), one or more phospholipids at 5 to 40 mol % (e.g., at 15 to 35 mol %), one or more sterol lipids at 15 to 50 mol % (e.g., at 30 to 50 mol %), DMG-PEG 2000 at 0 to 10 mol % (e.g., at 0 to 5 mol %), and DSPE-PEG 2000-maleimide at 0.01 to 1 mol %, and wherein the VHH is conjugated to the LNP.

In some aspects, provided herein is a circular ribonucleic acid (circRNA) therapeutic for targeted delivery comprising: a circular RNA comprising a ribosome recruiting element and a coding sequence encoding a chimeric antigen receptor (CAR) comprising an anti-CD19 antibody or antigen-binding fragment thereof, wherein the circRNA is encapsulated in an LNP, wherein a targeting moiety is conjugated to the LNP, wherein the targeting moiety is an anti-CD5 VHH, the anti-CD5 VHH comprising CDR1, CDR2, and CDR3 sequences from SEQ ID NO: 89, wherein the LNP comprises one or more ionizable lipids at 30 to 49 mol %, one or more phospholipids at 5 to 40 mol %, one or more sterol lipids at 15 to 50 mol %, a DMG-PEG 2000 lipid at 0 to 10 mol %, and a DSPE-PEG 2000-maleimide lipid at 0.01 to 1 mol %, and wherein the VHH is conjugated to the LNP.

In some aspects, provided herein is a circular ribonucleic acid (circRNA) therapeutic for targeted delivery comprising: a circular RNA comprising a ribosome recruiting element and a coding sequence encoding a chimeric antigen receptor (CAR) comprising an anti-CD19 antibody or antigen-binding fragment thereof, wherein the circRNA is encapsulated in an LNP, wherein a targeting moiety is conjugated to the LNP, wherein the targeting moiety is an anti-CD5 VHH, the anti-CD5 VHH comprising CDR1, CDR2, and CDR3 sequences from SEQ ID NO: 89, wherein the LNP comprises one or more ionizable lipids at 30 to 45 mol %, one or more phospholipids at 15 to 35 mol %, one or more sterol lipids at 30 to 50 mol %, a DMG-PEG 2000 lipid at 0 to 5 mol %, and a DSPE-PEG 2000-maleimide lipid at 0.01 to 1 mol %, and wherein the VHH is conjugated to the LNP.

In some embodiments, the VHH has a length of 110 to 130 (e.g., 115 to 125) amino acids. In some embodiments, the VHH has a length of 110 to 130 amino acids. In some embodiments, the VHH has a length of 115 to 125 amino acids. In some embodiments, the VHH has a length of about 121 amino acids.

In some embodiments, the VHH is conjugated to the LNP via a thiol-maleimide conjugation, and the thiol-maleimide conjugation is between the DSPE-PEG 2000-maleimide and a conjugation handle that is covalently attached to the VHH.

In some embodiments, the N-terminus of the conjugation handle is covalently attached to the C-terminus of the VHH.

In some embodiments, the VHH with the attached conjugation handle has a length of 120 to 140 amino acids (e.g., 125 to 135 amino acids). In some embodiments, the VHH with the attached conjugation handle has a length of 120 to 140 amino acids. In some embodiments, the VHH with the attached conjugation handle has a length of 125 to 135 amino acids.

In some embodiments, the VHH with the attached conjugation handle has a length of about 129 amino acids.

In some embodiments, the VHH comprises SEQ ID NO: 89.

In some embodiments, the conjugation handle comprises SEQ ID NO: 120.

In some embodiments, the sequence of the VHH with the attached conjugation handle comprises SEQ ID NO: 122.

In some embodiments, the sequence of the VHH with the attached conjugation handle is SEQ ID NO: 122.

In some embodiments, the LNP comprises the one or more ionizable lipids, the one or more phospholipids, the one or more sterol lipids, the DMG-PEG 2000 lipid, and the DSPE-PEG 2000-maleimide lipid at a ratio of 39:20:39:1.9:0.1 mol %.

In some embodiments, the one or more ionizable lipids is Compound 7.

In some embodiments, the one or more phospholipids is 1,2-distearoyl-snglycero-3-phosphocholine (DSPC).

In some embodiments, the one or more sterol lipids is cholesterol.

In some embodiments, provided herein is a circRNA therapeutic of the present disclosure comprising a circRNA sequence having at least 85% identity to SEQ ID NO: 264.

In some embodiments, provided herein is a circRNA therapeutic of the present disclosure comprising a circRNA sequence according to SEQ ID NO: 264.

In some aspects, provided herein is a circRNA therapeutic comprising (i) a circular RNA (circRNA) comprising a coding sequence encoding a CD19 CAR, wherein the circRNA is encapsulated in an LNP, (ii) the LNP, wherein the LNP comprises Compound 7 at 39 mol %, DSPC at 20 mol %, Cholesterol at 39 mol %, DMG-PEG 2000 lipid at 1.9 mol %, and DSPE-PEG 2000-maleimide lipid at 0.1 mol %, and (iii) a VHH with a conjugation handle comprising the sequence of SEQ ID NO:122, wherein the VHH with the conjugation handle is attached to the LNP via thiol-maleimide conjugation of the cysteine in the conjugation handle with the DSPE-PEG 2000-maleimide lipid.

In some embodiments, the circRNA comprises a sequence according to SEQ ID NO: 264.

In some aspects, provided herein is a circRNA therapeutic, comprising: (i) a circRNA comprising in order: (a) a 5′ exon fragment and a 5′ UTR, (b) an IRES having at least 85% identity to SEQ ID NO: 1, (c) a coding sequence encoding a chimeric antigen receptor (CAR) that targets CD19, (d) a 3′ UTR and a 3′ exon fragment, and (ii) an anti-CD5 T cell targeting moiety comprising the complementarity determining region (CDR) sequences CDR1, CDR2, and CDR3, wherein the CDR1, the CDR2, and the CDR3 amino acid sequences are GFTFSMYS (SEQ ID NO: 64), ISTGARDT (SEQ ID NO: 65), and GDLRYGPDGYDY (SEQ ID NO: 66), respectively, wherein the circRNA is encapsulated in a lipid nanoparticle, and wherein an anti-CD5 T cell targeting moiety is conjugated to the lipid nanoparticle.

In some embodiments, the coding sequence encoding a CAR comprises SEQ ID NO: 125.

In some embodiments, the CAR comprises an anti-CD19 scFv, a CD8a hinge, a CD8a transmembrane domain, a 4-1BB costimulatory domain, and a CD3 zeta signaling domain.

In some embodiments, the CAR comprises an amino acid sequence of SEQ ID NO: 58.

In some embodiments, the anti-CD19 scFv comprises a VL sequence of

(SEQ ID NO: 54)
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEIT,

linked by a linker having the amino acid sequence of GGGGSGGGGSGGGGS (SEQ ID NO: 56), and a VH sequence of

(SEQ ID NO: 55)
EVKLQESGPGLVAPSQSLSVTCTVSGVSLPDYGVSWIRQPPRKGLEWLGVIWGSETTYY
NSALKSRLTIIKDNSKSQVFLKMNSLQTDDTAIYYCAKHYYYGGSYAMDYWGQGTSVT
VSS.

In some aspects, provided herein isa circular ribonucleic acid (circRNA) comprising in order: (a) a 5′ exon fragment and 5′ untranslated region (UTR), (b) an Internal Ribosome Entry Site (IRES) comprising a sequence at least 85% identical to any one of SEQ ID NO: 1-11, (c) a coding sequence encoding a chimeric antigen receptor (CAR) comprising an anti-CD19 antibody or antigen-binding fragment thereof, (d) a 3′ UTR and a 3′ exon fragment, and (e) a single microRNA binding site within the 5′ UTR or the 3′ UTR.

In some embodiments, the 3′ UTR comprises the single microRNA binding site.

In some embodiments, the microRNA binding site comprises SEQ ID NO: 40.

In some embodiments, the single microRNA binding site is flanked by an upstream spacer and a downstream spacer, and wherein the sequence of each of the upstream spacer and the downstream spacer has a hydropathy index between −0.6 to −0.3.

In some embodiments, the sequence of the upstream spacer has a hydropathy index of −0.6 to −0.4.

In some embodiments, the sequence of each of the upstream spacer and downstream spacer has a hydropathy index of −0.6 to −0.5.

In some embodiments, each of the upstream spacer and downstream spacer comprise consecutive adenines but do not comprise more than five (5) consecutive adenines (As).

In some embodiments, each of the upstream spacer and downstream spacer comprise consecutive adenines but do not comprise more than four (4) consecutive adenines (As).

In some embodiments, each of the upstream spacer and downstream spacer comprise consecutive adenines but do not comprise more than three (3) consecutive adenines (As).

In some aspects, provided herein is a circular ribonucleic acid (circRNA) comprising, in order: (a) a 5′ exon fragment and a 5′ untranslated region (UTR), (b) an Internal Ribosome Entry Site (IRES), (c) a coding sequence encoding a chimeric antigen receptor (CAR) comprising an anti-CD19 antibody or antigen-binding fragment thereof, and (d) a 3′ UTR comprising an upstream spacer, a microRNA binding site, and a downstream spacer, and a 3′ exon fragment, wherein each of the upstream spacer and downstream spacer comprise multiple instances of consecutive adenines (As) but do not comprise more than five (5) consecutive adenines (As).

In some embodiments, each of the upstream spacer and the downstream spacer do not comprise more than four (4) consecutive adenines (As).

In some embodiments, each of the upstream spacer and the downstream spacer do not comprise more than three (3) consecutive adenines (As).

In some embodiments, the sequence of each of the upstream spacer and the downstream spacer has a hydropathy index between −0.6 to −0.3.

In some embodiments, the sequence of the upstream spacer has a hydropathy index of −0.6 to −0.4.

In some embodiments, each of the upstream spacer and downstream spacer comprise one or more instances of a cytosine (C) interrupting consecutive As.

In some embodiments, each of the upstream spacer and downstream spacer comprise no uracil.

In some embodiments, the upstream spacer sequence and the downstream spacer sequence consist only of As and Cs.

In some embodiments, each of the upstream spacer and downstream spacer have a length of not more than 18 nucleotides.

In some embodiments, each of the upstream spacer and downstream spacer have a length of 12-18 nucleotides.

In some embodiments, each of the upstream spacer and downstream spacer have a length of 12-15 nucleotides.

In some embodiments, the upstream spacer and the downstream spacer do not differ in length by more than 4 nucleotides.

In some embodiments, the upstream spacer and the downstream spacer do not differ in length by more than 3 nucleotides.

In some embodiments, the upstream spacer and the downstream spacer do not differ in length by more than 2 nucleotides.

In some embodiments, the microRNA binding site is a microRNA-122 (miR-122) binding site.

In some embodiments, the 3′ UTR comprises

(SEQ ID NO: 349)
TAATAGaacaaacacaaacCAAACACCATTGTCACACTCCAaacaacaaacaaacaACCACACAAA
TGGTCGCCGA.

In some embodiments, the 5′ UTR comprises

(SEQ ID NO: 348)
TCGGCGACCATTTGTGTGGTGAGCTCTAAGTGTTGATCAGCGGGCGCAG
CTGCATATGACCTCTGCGTGTGCCTGGTAATTGACTAAGATATC.

In some embodiments, the 5′ UTR comprises SEQ ID NO: 348 and the 3′ UTR comprises SEQ ID NO: 349.

In some embodiments, the 5′ exon fragment comprises SEQ ID NO: 382 and a 3′ exon fragment comprises SEQ ID NO: 389.

In some embodiments, the IRES comprises a sequence having at least 90% identity to any one of SEQ ID NO: 1-6.

In some embodiments, the IRES comprises the sequence of SEQ ID NO: 1.

In some embodiments of the circRNA, wherein: the coding sequence further encodes a CD8 signal peptide, and the CAR further comprises a CD8 hinge, a CD8 transmembrane domain, a 4-1BB costimulatory domain, and a CD3 zeta signaling domain.

In some embodiments, the anti-CD19 antibody or antigen-binding fragment thereof comprises a VL sequence of SEQ ID NO: 54, and a VH sequence of SEQ ID NO: 55.

In some embodiments, the VL and the VH are joined by a linker.

In some embodiments, the linker comprises SEQ ID NO: 56.

In some embodiments, the coding sequence encodes an amino acid sequence having at least 90% identity to SEQ ID NO: 58.

In some embodiments, the coding sequence encodes an amino acid sequence having at least 95% identity to SEQ ID NO: 58.

In some embodiments, the coding sequence encodes an amino acid sequence of SEQ ID NO: 58.

In some aspects, provided herein is a circRNA comprising a sequence having at least 85% identity to SEQ ID NO: 264.

In some embodiments, provided herein is a circRNA comprising a sequence having at least 90% identity to SEQ ID NO: 264.

In some embodiments, provided herein is a circRNA comprising a sequence having at least 95% identity to SEQ ID NO: 264.

In some embodiments, provided herein is a circRNA comprising a sequence having at least 98% identity to SEQ ID NO: 264.

In some embodiments, provided herein is a circRNA comprising a sequence having at least 99% identity to SEQ ID NO: 264.

In some embodiments, the circRNA comprises SEQ ID NO: 264.

In some embodiments, provided herein is a circRNA therapeutic, comprising circRNA of the present disclosure.

In some embodiments, the circRNA is as provided by the present disclosure.

In some embodiments, the circRNA therapeutic comprises the circRNA of the present disclosure encapsulated in an LNP.

In some embodiments, the LNP is conjugated to a targeting moiety, wherein the targeting moiety binds to T cells.

In some embodiments, the targeting moiety comprises an anti-CD5 antibody or antigen-binding fragment thereof.

In some aspects, provided herein is a precursor RNA comprising, from 5′ to 3′ end: (i) a 5′ intron fragment and a 5′ exon fragment, (ii) a 5′ untranslated region (UTR), (iii) an IRES, (iv) a coding sequence encoding a chimeric antigen receptor (CAR) comprising an anti-CD19 antibody or antigen-binding fragment thereof, (v) a 3′ untranslated region (UTR), and (vi) a 3′ exon fragment and a 3′ intron fragment, wherein the 5′ and 3′ intron fragments are derived from a Twort-ORF142 Group I intron, and wherein the 3′ UTR comprises a single microRNA binding site.

In some embodiments, the single microRNA binding site is flanked by an upstream spacer and a downstream spacer.

In some embodiments, each of the upstream spacer and the downstream spacer comprise no more than five (5) consecutive adenines (As).

In some embodiments, each of the upstream spacer and the downstream spacer do not comprise more than four (4) consecutive adenines (As).

In some embodiments, each of the upstream spacer and the downstream spacer do not comprise more than three (3) consecutive adenines (As).

In some embodiments, the sequence of each of the upstream spacer and the downstream spacer has a hydropathy index between −0.6 to −0.3.

In some embodiments, the sequence of the upstream spacer has a hydropathy index of −0.6 to −0.3.

In some embodiments, each of the upstream spacer and downstream spacer comprise one or more instances of a cytosine (C) interrupting consecutive As.

In some embodiments, each of the upstream spacer and downstream spacer comprise no uracil.

In some embodiments, the upstream spacer sequence and the downstream spacer sequence consist only of As and Cs.

In some embodiments, each of the upstream spacer and downstream spacer have a length of not more than 18 nucleotides.

In some embodiments, each of the upstream spacer and downstream spacer have a length of 12-18 nucleotides.

In some embodiments, each of the upstream spacer and downstream spacer have a length of 12-15 nucleotides.

In some embodiments, the upstream spacer and the downstream spacer do not differ in length by more than 4 nucleotides.

In some embodiments, the upstream spacer and the downstream spacer do not differ in length by more than 3 nucleotides.

In some embodiments, the upstream spacer and the downstream spacer do not differ in length by more than 2 nucleotides.

In some embodiments, the microRNA binding site is a microRNA-122 (miR-122) binding site.

In some aspects, provided herein is a precursor RNA comprising, from 5′ to 3′ end: (i) a 5′ intron fragment and a 5′ exon fragment, (ii) a 5′ untranslated region (UTR), (iii) an IRES, (iv) a coding sequence encoding a chimeric antigen receptor (CAR) comprising an anti-CD19 antibody or antigen-binding fragment thereof, (v) a 3′ untranslated region (UTR), and (vi) a 3′ exon fragment and 3′ intron fragment, wherein the 5′ and the 3′ intron fragments are derived from a Twort-ORF142 Group I intron, wherein the 3′ UTR comprises a microRNA binding site flanked by an upstream spacer and a downstream spacer, and wherein each of the upstream spacer and the downstream spacer comprise no more than five (5) consecutive adenines (As).

In some embodiments, each of the upstream spacer and the downstream spacer comprise no more than four (4) consecutive adenines (As).

In some embodiments, each of the upstream spacer and the downstream spacer comprise no more than three (3) consecutive adenines (As).

In some embodiments, each of the upstream spacer and the downstream spacer comprise one or more instances of a cytosine (C) interrupting consecutive As.

In some embodiments, each of the upstream spacer and the downstream spacer comprise no uracil.

In some embodiments, each of the upstream spacer and the downstream spacer comprise 12-18 nucleotides.

In some embodiments, each of the upstream spacer and downstream spacer have a length of 12-15 nucleotides.

In some embodiments, the upstream spacer and the downstream spacer do not differ in length by more than 4 nucleotides.

In some embodiments, the upstream spacer and the downstream spacer do not differ in length by more than 3 nucleotides.

In some embodiments, the upstream spacer and the downstream spacer do not differ in length by more than 2 nucleotides.

In some embodiments, the microRNA binding site is a microRNA-122 (miR-122) binding site.

In some embodiments, the 5′ intron fragment comprises SEQ ID NO: 381.

In some embodiments, the 5′ exon fragment comprises SEQ ID NO: 382.

In some embodiments, the 5′ UTR comprises SEQ ID NO: 348.

In some embodiments, the IRES comprises a sequence having at least 90% identity to any one of SEQ ID NO: 1-6.

In some embodiments, the IRES comprises the sequence of SEQ ID NO: 1.

In some embodiments, the coding sequence encoding the CAR comprising the anti-CD19 antibody or antigen-binding fragment thereof comprises the sequence of SEQ ID NO: 355.

In some embodiments, the 3′ UTR comprises SEQ ID NO: 349 or 350.

In some embodiments, the 3′ exon fragment comprises SEQ ID NO: 389.

In some embodiments, the 3′ intron fragment comprises SEQ ID NO: 390.

In some embodiments, the 5′ UTR comprises SEQ ID NO: 348 and the 3′ UTR comprises SEQ ID NO: 349.

In some embodiments, the 5′ intron fragment and the 5′ exon fragment together comprises SEQ ID NO: 276 and the 3′ exon fragment and the 3′ intron fragment together comprises SEQ ID NO: 277.

In some aspects, provided herein is a precursor RNA comprising, from 5′ to 3′ end: (i) a 5′ intron fragment and a 5′ exon fragment together comprising SEQ ID NO: 276, (ii) a 5′ untranslated region (UTR), (ii) an IRES, (iii) a coding sequence encoding a chimeric antigen receptor (CAR) comprising an anti-CD19 antibody or antigen-binding fragment thereof, (iv) a 3′ untranslated region (UTR), and (v) a 3′ exon fragment and a 3′ intron fragment together comprising SEQ ID NO: 277.

In some embodiments, provided herein is a circular RNA circularized from the precursor RNA of the present disclosure.

In some embodiments, provided herein is a circular RNA (circRNA) therapeutic comprising the circular RNA of the present disclosure.

In some embodiments, provided herein is a method of depleting B cells comprising administering to a subject in need thereof the circRNA therapeutic of the present disclosure.

In some embodiments, provided herein is a method of treating a B cell-mediated disease comprising administering to a subject in need thereof the circRNA therapeutic of the present disclosure.

In some embodiments, the B cell-mediated disease is an autoimmune disease. In some embodiments, the autoimmune disease is one or more of lupus (e.g., systemic lupus erythematosus), an inflammatory myopathy, systemic sclerosis, rheumatoid arthritis, Sjogrens disease, multiple sclerosis (e.g., PPMS, RMS), myasthenia gravis, autoimmune hemolytic anemia, immune thrombocytopenia, and inflammatory bowel disease (e.g., Crohn's disease, ulcerative colitis).

In some embodiments, the B cell-mediated disease is cancer.

### BRIEF DESCRIPTION OF THE DRAWINGS

The drawings are for illustration purposes only and are not meant to be limiting.

FIG. 1A is a schematic showing a template DNA (e.g., plasmid), which is transcribed to synthesize an exemplary linear precursor RNA (from which the circular RNA of the present disclosure is derived). In some embodiments, as shown in FIG. 1B, autocatalytic self-splicing of two halves of a Permuted Group I Intron-Exon or PIE construct (e.g., a 5′ intron fragment and a 3′ intron fragment) leads to the formation of the single-stranded circular RNA (circRNA) drug substance of the present disclosure that encodes an anti-CD19 chimeric antigen receptor (CAR) for treatment of B-cell-driven diseases (e.g., autoimmune disease and cancer). The circRNA sequence comprises, for example, a 5′ exon fragment and 5′ untranslated region (UTR), a ribosome recruiting element (e.g., an internal ribosome entry site (IRES)), a coding sequence (CDS), and a 3′ UTR and 3′ exon fragment, with the 5′ and/or 3′ UTRs optionally containing one or more spacers, and the 5′ exon fragment and 3′ exon fragment are joined at a splice junction. As shown in more detail in FIG. 1C, after autocatalytic self-splicing of two halves of a PIE construct, the 5′ exon fragment and 3′ exon fragment are ligated, and the intron is excised from the circular RNA. The splice junction shows the boundary after self-splicing and circularization, leaving the 5′ exon fragment and 3′ exon fragment adjacent to each other at the splice junction. The nucleobase composition of the drug substance comprises adenine (represented by A's), cytosine (represented by C's), guanine (represented by G's), and uracil (represented by T's or U's; in the context of RNA, it is understood that T's and U's both represent uracil). In some embodiments, the circRNA is 2000 to 2700 nucleotides in length. In some embodiments, the circRNA is 2000 to 2500 nucleotides in length. In some embodiments, the circRNA is 2200 to 2500 nucleotides in length. In some embodiments, the circRNA is 2300 to 2400 nucleotides in length. In some embodiments, the circRNA is 2300 to 2350 nucleotides in length. In some embodiments, the circRNA is about 2000 nucleotides long. In some embodiments, the circRNA is about 2100 nucleotides long. In some embodiments, the circRNA is about 2200 nucleotides long. In some embodiments, the circRNA is about 2300 nucleotides long. In some embodiments, the circRNA is about 2330 nucleotides long. In some embodiments, the circRNA is 2332 nucleotides long. In some embodiments, the circRNA is about 2400 nucleotides long. In some embodiments, the circRNA is about 2500 nucleotides long.

FIG. 1D shows a schematic of a circRNA drug product of the present disclosure comprising a circRNA encoding for, for example, an anti-CD19 CAR, encapsulated by a lipid nanoparticle (LNP) containing an exemplary ionizable lipid (e.g., Compound 7) conjugated to a targeting moiety, for example, an anti-CD5 VHH (nanobody) for robust targeted delivery to T cells, including CD4⁺ and CD8⁺ T cells.

FIG. 1E is a schematic of the circRNA that shows an exemplary mechanism of action of the circRNA drug product without wishing to be bound by any particular theory. Although not bound by any mechanism, the targeting moiety, e.g., anti-CD5 nanobody, allows specific targeting of the circRNA encapsulated in the LNP to the targeting receptor, e.g., T cells that express CD5. Thus, the circRNA encoding the anti-CD19 CAR is delivered to the T cell, which uses the T cell's own machinery to translate the anti-CD19 CAR and express the CAR on the surface of the T cell. The CAR T cell then binds to its target, e.g., B cells expressing CD19, via the antigen-binding domain of the CAR. Antigen binding to the CAR triggers intracellular signaling domains, leading to T cell activation that induces secretion of cytokines and cytotoxic molecules. These molecules destroy and eliminate target B cells, resulting in B cell depletion, which is the intended on-target effect for the therapeutic treatment of B cell-mediated diseases.

FIG. 2A is a plot of area under the curve showing reporter gene expression from circRNA constructs comprising multiple exemplary IRESs. The performance of various IRESs was evaluated in vitro in comparison to a reference IRES (IRES090).

FIG. 2B is a plot of area under the curve (average mean fluorescence per cell) for circRNA constructs comprising various exemplary IRESs.

FIG. 2C is a graph of percentage of CAR-positive activated and resting T cells (of total live CD3-positive cells), two days post-transfection of circRNA constructs comprising various exemplary IRESs.

FIG. 2D is a graph of total CAR molecules per CAR T cell in resting and activated T cells showing CAR expression per cell, two days post-transfection of circRNA constructs comprising various exemplary IRESs.

FIG. 2E is a graph of gene expression (gMFI) of circRNA comprising exemplary IRESs on day 1 post-transfection of the circRNA comprising exemplary IRESs relative to a reference IRES (IRES090). The different data points represent different T cell donors.

FIG. 2F is a graph of in vivo anti-CD19 CAR expression in CD3⁺ T cells from spleens of hPBMC (human peripheral blood mononuclear cells)-engrafted NSG DKO mice as a percentage of total CD3⁺ T cells at 48 hours after administration of circRNA (with various exemplary IRESs) formulated in LNP.

FIG. 2G is a graph of in vivo anti-CD19 CAR expression in CD3⁺ T cells from bone marrow of hPBMC-engrafted NSG DKO mice as a percentage of total CD3⁺ T cells at 48 hours after administration of circRNA (with various exemplary IRESs) formulated in LNP.

FIG. 2H is a graph of in vivo anti-CD19 CAR expression in CD3⁺ T cells from peripheral blood of hPBMC-engrafted NSG DKO mice as a percentage of total CD3+ T cells at 48 hours after administration of circRNA (with various exemplary IRESs) formulated in LNP.

FIG. 2I is a graph of proportion of B cells to mean B cell count of vehicle control in spleens of hPBMC-engrafted NSG DKO mice at 48 hours after administration of circRNA (with various exemplary IRESs) formulated in LNP.

FIG. 2J is a graph of proportion of B cells to mean B cell count of vehicle control in bone marrow of hPBMC-engrafted NSG DKO mice at 48 hours after administration of circRNA (with various exemplary IRESs) formulated in LNP.

FIG. 2K is a graph of proportion of B cells to mean B cell count of vehicle control in peripheral blood of hPBMC-engrafted NSG DKO mice at 48 hours after administration of circRNA (with various exemplary IRESs) formulated in LNP.

FIG. 3A is a schematic of circRNA showing, amongst the other elements described in FIG. 1A, spacers within the 5′ UTR and the 3′ UTR.

FIG. 3B is a graph of area under the curve of eGFP expression in HEK293T cells upon insertion of exemplary 3′ spacers in the circRNA construct.

FIG. 3C is a graph of CAR expression in human T cells from a circRNA comprising CAA40 spacer in the 3′ UTR. Three replicates are shown. A “no RNA” control and a scrambled IRES (negative control) were also tested.

FIG. 4A is a graph of percent CAR expression in T cells from three donors transfected with a circRNA encapsulated in a LNP comprising Compound 7, with an anti-CD5 VHH319 targeting moiety, wherein the circRNA had no miR-122 binding site, one miR-122 binding site, or three miR-122 binding sites in the 3′ UTR.

FIG. 4B is a graph of CAR expression (measured as geometric Mean Fluorescence Intensity, gMFI) in T cells from three donors transfected with a circRNA encapsulated in a LNP comprising Compound 7, with an anti-CD5 VHH319 targeting moiety, wherein the circRNA had no miR-122 binding site, one miR-122 binding site, or three miR-122 binding sites in the 3′ UTR.

FIG. 4C is a graph of percent CAR expression in Huh7 immortalized hepatocytes (off-target liver cells) transfected with a circRNA encapsulated in a LNP comprising Compound 7, with an anti-CD5 VHH319 targeting moiety, wherein the circRNA had no miR-122 binding site, one miR-122 binding site, or three miR-122 binding sites in the 3′ UTR.

FIG. 4D is a graph of CAR expression (gMFI) in Huh7 immortalized hepatocytes (off-target liver cells) transfected with a circRNA encapsulated in a LNP comprising Compound 7, with an anti-CD5 VHH319 targeting moiety, wherein the circRNA had no miR-122 binding site, one miR-122 binding site or three miR-122 binding sites in the 3′ UTR.

FIG. 4E is a graph of luciferase expression in spleens of CD34⁺ HSC-engrafted humanized NSG mice at 24 hours after administration of various circular RNA constructs comprising one or three miR-122 binding sites, relative to circRNA constructs comprising no miR-122 binding site in the 3′ UTR.

FIG. 4F is a graph of luciferase expression in livers of CD34⁺ HSC-engrafted humanized NSG mice at 24 hours after administration of various circular RNA constructs comprising one or three miR-122 binding sites, relative to circRNA constructs comprising no miR-122 binding site in the 3′ UTR.

FIG. 4G shows a schematic of the miR-122 binding site in the UTR of the circRNA constructs with re-designed flanking sequences (miR-122_1 spacer and miR-122_2 spacer).

FIG. 4H is a graph of percent circularization of in vitro transcribed RNA. As shown in FIG. 4H, inclusion in the 3′ UTR of either miR-122_1 spacer (within SEQ ID NO: 228) or miR-122_2 spacer (within SEQ ID NO: 264) showed comparable circularization, with an improvement seen with the inclusion of miR-122_2 spacer as compared to the miR-122_1 spacer. Overall, these results showed that interrupting poly A tracts (for example, by replacing A with cytosine) resulted in some improvement in circularization of RNA.

FIG. 4I is a graph of hepatic knockdown by circRNA comprising UTR with a miR-122_2 spacer relative to miR-122_1 spacer. In Huh7 hepatocytes as measured by flow cytometry, 24 hours after transfection, percent CAR expression was comparably decreased by inclusion of either miR-122_1 spacer or miR-122_2 spacer relative to no miR-122 binding site in the 3′ UTR of the circRNA. “Full control” refers to a no treatment control (with no buffer or reagent addition, no RNA, and no electroporation). “EP Only” refers to an empty electroporation control where cells were electroporated in the absence of RNA).

FIG. 4J is a graph of hepatic knockdown by circRNA comprising UTR with a miR-122_2 spacer relative to miR-122_1 spacer. In Huh7 hepatocytes as measured by flow cytometry, 24 hours after transfection, CAR gMFI levels were comparably decreased by inclusion of either miR-122_1 spacer or miR-122_2 spacer relative to no miR-122 binding site. “Full control” refers to a no treatment control (with no buffer or reagent addition, no RNA and no electroporation). “EP Only” refers to an empty electroporation control where cells were electroporated in the absence of RNA.

FIG. 4K is a graph of anti-CD19 CAR positive cells as a percentage of CD3⁺ T cells from spleens of CD34⁺ HSC-engrafted humanized NSG mice at 24 hours after administration of circRNA constructs formulated in LNP. This data compares an exemplary circRNA construct without a miRNA binding site (SEQ ID NO: 175) and a circRNA construct with one miRNA-122 binding site in the 3′ UTR of the circRNA (SEQ ID NO: 264).

FIG. 4L is a graph of anti-CD19 CAR expression in blood as a percentage of total CD3+ T cells following administration of circRNA including no miRNA binding site or miR-122_2 spacer in the 3′ UTR; the circRNA is conjugated to an anti-CD5 targeting moiety (VHH319) and encapsulated in two exemplary LNPs, (LNP-11 and LNP-15, as described in Table 16).

FIG. 4M is a graph of proportion of B cells relative to vehicle control in blood following administration of circRNA including no miRNA binding site (SEQ ID NO: 175) or one miR-122_2 spacer in the 3′ UTR (SEQ ID NO: 264). The circRNAs were encapsulated in LNPs conjugated to an anti-CD5 targeting moiety (VHH319) and were formulated in two exemplary formulations LNP-11 and LNP-15 (as described in Table 16).

FIG. 4N is a graph of anti-CD19 CAR expression in spleen as a percentage of total CD3+ T cells following administration of circRNA including no miRNA binding site (SEQ ID NO: 175) or one miR-122_2 spacer in the 3′ UTR (SEQ ID NO: 264). The circRNAs were encapsulated in LNPs conjugated to an anti-CD5 targeting moiety (VHH319) and were formulated in two exemplary formulations LNP-11 and LNP-15 (as described in Table 16).

FIG. 4O is a graph of proportion of B cells relative to vehicle control in the spleen following administration of circRNA including no miRNA binding site (SEQ ID NO: 175) or one miR-122_2 spacer in the 3′ UTR (SEQ ID NO: 264). The circRNAs were encapsulated in LNPs conjugated to an anti-CD5 targeting moiety (VHH319) and were formulated in two exemplary formulations LNP-11 and LNP-15 (as described in Table 16).

FIG. 5 is a graph of CAR expression measured by flow cytometry showing that various exemplary circRNA comprising codon optimized coding sequences demonstrated CAR expression in activated T cells in vitro.

FIG. 6A is a schematic of exemplary anti-CD19 CAR architectures comprising an FMC63 scFv, and various exemplary transmembrane domains, costimulatory domains, and signal domains; and exemplary signaling peptides.

FIG. 6B is a graph of anti-CD19 CAR expression in CD3⁺ T cells from spleens of CD34⁺ HSC-engrafted NSG humanized mice at 24 hours after administration of circRNA (encoding various exemplary CAR architectures) encapsulated in targeted LNP.

FIG. 6C is a graph of anti-CD19 CAR expression in circulating CD3⁺ T cells of CD34⁺ HSC-engrafted NSG humanized mice at 24 hours after administration of circRNA (encoding various exemplary CAR architectures) encapsulated in targeted LNP.

FIG. 6D is a graph of the number of anti-CD19 CAR positive T cells per 5×10⁵ splenocytes from spleens of CD34⁺ HSC-engrafted NSG humanized mice at 24 hours after administration of circRNA (encoding with exemplary CAR architectures) encapsulated in targeted LNP.

FIG. 6E is a graph of proportion of B cells to mean B cell count of vehicle control present in spleens of CD34⁺ HSC-engrafted NSG humanized mice at 24 hours after administration of circRNA (encoding exemplary CAR architectures) encapsulated in targeted LNP.

FIG. 6F is a graph of proportion of B cells to mean B cell count of vehicle control in peripheral blood of CD34⁺ HSC-engrafted NSG humanized mice at 24 hours after administration of circRNA (encoding exemplary CAR architectures) encapsulated in targeted LNP.

FIG. 7A is a graph of total mGL+ cells relative to percentage of CD3+ T cells 24 hours after administration of an exemplary circRNA encapsulated in an LNP (“RNA-LNP”) at 0.5 mg/kg, wherein the LNP comprises 0.1% C18 PEG (LNP-19), 0.5% C18 PEG (LNP-20) or 1% C18 PEG (LNP-21).

FIG. 7B is a graph showing mGL expression in liver (off-target) relative to spleen 24 hours after administration of an exemplary RNA-LNP at 0.5 mg/kg, wherein the LNP comprises 0.1% C18 PEG (LNP-19), 0.5% C18 PEG (LNP-20) or 1% C18 PEG (LNP-21), as described in Table 16.

FIG. 7C is a graph showing total luciferase flux in the liver of mice administered circRNA encoding a reporter protein (luciferase) encapsulated in non-targeted LNP-1, LNP-18 and LNP-22, as described in Table 16.

FIG. 7D is a graph showing total luciferase flux in the spleen of mice administered circRNA encoding a reporter protein (luciferase) encapsulated in non-targeted LNP-1, LNP-18 and LNP-22, as described in Table 16.

FIG. 7E is a graph showing circRNA levels in the liver of the mice, normalized to the mean value for circRNA encapsulated in LNP-1, as described in Table 16.

FIG. 7F is a graph showing circRNA levels in the spleen of the mice, normalized to the mean value for circRNA encapsulated in LNP-1, as described in Table 16.

FIG. 8A is a graph showing mGreenLantern (mGL) expression in CD45.2+ leukocytes in various tissues, including bone marrow, lungs, lymph nodes, peripheral blood, and spleen.

FIG. 8B shows cellular distribution as percentage of mGL+ cells in tissues after administration of RNA-LNP, wherein the LNP comprises Compound 2.

FIG. 8C shows cellular distribution as percentage of mGL+ cells in tissues after administration of RNA-LNP comprising mGreenLantern (mGL) and firefly luciferase (ffluc) reporter RNAs, wherein the LNP comprised Compound 7.

FIG. 8D is a graph that shows anti-CD19 CAR expression in spleen as a percentage of CD3+ T cells. RNA-LNPs (SEQ ID NO: 175), wherein the LNPs comprised both Compound 2 and Compound 7 showed targeting to spleen with the inclusion of an anti-CD5 targeting moiety (VHH319) relative to the corresponding base LNP alone (base LNP refers to LNP with no targeting moiety).

FIG. 8E is a graph that shows corresponding B cell depletion in spleen, after administration of exemplary RNA-LNPs (wherein LNPs comprised both Compound 2 and Compound 7 comprising SEQ ID NO: 175), was comparable.

FIG. 8F is a graph that shows anti-CD19 CAR positive cells relative to percentage of total CD3+ T cells in multiple studies carried out with RNA encapsulated in a base LNP, which refers to a non-targeted LNP, compared to a RNA-targeted LNP comprising SEQ ID NO: 175 and Compound 2. Also included was a PBS-treated vehicle control.

FIG. 8G is a graph that shows anti-CD19 CAR MFI relative to total CD3+ T cells in multiple studies carried out with RNA-LNP comprising SEQ ID NO: 175 and Compound 2.

FIG. 8H is a graph that shows B cell depletion in multiple studies carried out with RNA-LNP comprising SEQ ID NO: 175 and Compound 2.

FIG. 8I-FIG. 8K are flow cytometry data showing the increased targeting efficiency of circRNA formulated in a CD5-targeted LNP targeting to CD4+ and CD8+ T cells in the spleen of mice. The mice were administered via intravenous administration 0.5 mg/kg of circRNA (encoding mGL) formulated in an anti-CD5 targeted LNP or a vehicle control, and the level of mGL expression was measured.

FIG. 8L-FIG. 8N are flow cytometry data showing the increased targeting efficiency of circRNA formulated in a CD5-targeted LNP targeting to CD4+ and CD8+ T cells in the lymph node of mice. The mice were administered via intravenous administration 0.5 mg/kg of circRNA (encoding mGL) or a vehicle control, and the level of mGL expression was measured.

FIG. 9A is a graph of mGreenLantern gMFI expression in Jurkat T cells using circRNA (encoding an mGreenLantern reporter) encapsulated by LNP with various exemplary LNP formulations described in Table 16 and shows that increasing the DSPC from the standard amount of about 10% DSPC to about 15-25% DSPC increased mGL expression and increasing to greater than 25% DSPC further increased the mGL expression in Jurkat T cells.

FIG. 9B is a graph of % mGL positive (among live cells) in Jurkat T cells by circRNA encoding mGreenLantern reporter encapsulated by LNP, with various exemplary LNP formulations described in Table 16. A reference 10% DSPC showed about 75% mGL positive cells. At 15-25% DSPC, most formulations tested showed increased reporter expression compared to the reference 10% DSPC. At greater than 25% DSPC, all LNP formulations tested showed greater than 90% mGL positive cells.

FIG. 9C is a graph that shows anti-CD19 CAR positive cells relative to percentage of mCD45+ cells.

FIG. 9D is a graph that shows B cell depletion in multiple studies carried out using various exemplary LNP formulations, including formulations comprising Compound 7.

FIG. 10A is a schematic that shows exemplary targeting moiety formats, e.g., single-chain variable fragment (scFv), VHH and full-length antibodies.

FIG. 10B is a graph of percent B cells remaining after 24 hours upon administration of RNA-LNPs comprising various exemplary targeting moieties (anti-CD2, anti-CD5, anti-CD7 and anti-CD8) relative to vehicle control. Anti-CD5 VHH performed as well as full-length anti-CD2, anti-CD7 and anti-CD8 antibodies.

FIG. 10C is a graph showing proportion of B cells remaining 24 hours after administration of RNA-LNP comprising two exemplary targeting moieties, anti-CD5 and anti-CD8 VHHs, with a “G3S-Cys-AAA” (SEQ ID NO: 120) conjugation handle or “G4S-Cys-AAA” (SEQ ID NO: 343) conjugation handle.

FIG. 10D is a graph showing anti-CD19 CAR MFI relative to CD4+ T cells by administration of RNA-LNP comprising RNA of SEQ ID NO: 175, exemplary anti-CD5 VHH319 comprising a “G3S-Cys-AAA” (SEQ ID NO: 120) conjugation handle and a reference conjugation handle.

FIG. 10E is a graph showing anti-CD19 CAR MFI relative to CD8+ T cells by administration of RNA-LNP comprising exemplary anti-CD5 VHH319 comprising a “G3S-Cys-AAA” (SEQ ID NO: 120) conjugation handle and a reference conjugation handle.

FIG. 10F is a graph showing CD69 MFI as a marker of T cell activation relative to CD4+ T cells by administration of RNA-LNP comprising exemplary anti-CD5 VHH319 comprising a “G3S-Cys-AAA” (SEQ ID NO: 120) conjugation handle and a reference conjugation handle.

FIG. 10G is a graph showing CD69 MFI as a marker of T cell activation relative to CD8+ T cells by administration of RNA-LNP comprising exemplary anti-CD5 VHH319 comprising a “G3S-Cys-AAA” (SEQ ID NO: 120) conjugation handle and a reference conjugation handle.

FIG. 11A is a graph showing the percentage of anti-CD19 CAR+ cells relative to total human CD3+ T cells 24 hours after RNA-LNP administration for LNPs with various exemplary anti-CD5 VHH nanobodies as targeting moieties: VHH319, VHH322, VHH325, VHH329, VHH330 and VHH331 described in Table 10.

FIG. 11B is a graph showing the proportion of B cells remaining after 24 hours of RNA-LNP administration relative to vehicle control for LNPs with various exemplary anti-CD5 VHH nanobodies as targeting moieties: VHH319, VHH322, VHH325, VHH329, VHH330 and VHH331 described in Table 10.

FIG. 12A is a schematic of various conjugation handles used to conjugate the targeting moiety to the delivery vehicle, e.g., LNPs (sequences shown in Table 12).

FIG. 12B is a graph showing percentage of anti-CD19 CAR+ cells relative to total CD3+ T cells in blood.

FIG. 12C is a graph showing percentage of anti-CD19 CAR CD3+ T cells (MFI) in blood.

FIG. 12D is a graph showing percentage of anti-CD19 CAR+ cells relative to total CD3+ T cells in spleen.

FIG. 12E is a graph showing percentage of anti-CD19 CAR CD3+ T cells (MFI) in spleen.

FIG. 12F is a graph showing number of anti-CD19 CAR+ T cells per 5×10⁵ splenocytes.

FIG. 12G is a graph showing proportion of B cells remaining in spleen relative to vehicle control when administered an anti-CD19 RNA-LNP (SEQ ID NO: 175), with exemplary modified anti-CD5 VHH319 targeting moieties—an anti-CD5 VHH319 comprising a reference conjugation handle, for example, at the C-terminal, and an anti-CD5 VHH319 comprising a “G3S-CYS-AAA” (SEQ ID NO: 120) conjugation handle were compared.

FIG. 12H is a graph showing proportion of B cells remaining in spleen relative to vehicle control when administered an anti-CD19 RNA-LNP with exemplary modified anti-CD5 VHH319 targeting moieties—an anti-CD5 VHH319 comprising S7C, S70C and S82bC, and a control anti-CD5 VHH319 comprising an exemplary C-terminal reference conjugation handle.

FIG. 12I is a graph showing proportion of B cells remaining in blood relative to vehicle control when administered an LNP-anti-CD19 RNA comprising exemplary modified anti-CD5 VHH319 targeting moieties—VHH319 comprising S7C, S70C and S82bC and a control VHH319 comprising a reference conjugation handle.

FIG. 12J is a graph showing anti-CD19 CAR expression in spleen as a percentage of CD3+ T cells when administered anti-CD19 circRNA conjugated to an LNP using various exemplary conjugation chemistries, namely, 0.1% PEG maleimide C-terminal conjugation, with and without a histidine tag, 0.1% PEG-maleimide C-terminal “G3S-Cys-AAA” (SEQ ID NO: 120) or G4SG4S-Cys-AS (SEQ ID NO: 345) conjugation handle, 0.1% PEG maleimide S82bC conjugation with and without histidine tag and a 0.5% PEG maleimide S82bC conjugation without histidine tag relative to a vehicle control and base LNP control.

FIG. 12K is a graph showing proportion of B cells remaining in spleen relative to vehicle control when administered anti-CD19 circRNA conjugated to an LNP comprising exemplary conjugation chemistries, namely, 0.1% PEG maleimide C-terminal conjugation, with and without a histidine tag, 0.1% PEG-maleimide C-terminal “G3S-Cys-AAA” (SEQ ID NO: 120) conjugation handle, 0.1% PEG maleimide S82bC conjugation with and without histidine tag and a 0.5% PEG maleimide S82bC conjugation without histidine tag relative to a vehicle control and base LNP control.

FIG. 13A shows a schematic of exemplary conjugation chemistries in conjugating a targeting moiety to the delivery vehicle, e.g., LNP.

FIG. 13B shows flow cytometry data showing eGFP positive cells in conjugates using thiol-maleimide and azide-DBCO conjugations relative to an unconjugated (base) LNP control.

FIG. 13C shows flow cytometry data showing mGreenLantern positive cells in conjugates using sortase-catalyzed LPXT/G-glycine conjugation, wherein X is any amino acid residue.

FIG. 14A is a graph of mGL positive cells as a percentage of total CD45.2+ leukocytes.

FIG. 14B is a graph of cellular biodistribution of gene expression in cells administered linear RNA in an LNP formulation comprising Compound 2.

FIG. 14C is a graph of cellular biodistribution of gene expression in cells administered circular RNA in an LNP formulation comprising Compound 2.

FIG. 14D is a graph of anti-CD19 CAR positive T cells derived from flow cytometry data shown in FIG. 14E on day 1, day 4, and day 7 comparing the number of CAR positive cells from linear RNA (SEQ ID NO: 275) and circRNA (SEQ ID NO: 230).

FIG. 14E is flow cytometry data from day 1, day 4, and day 7 comparing anti-CD19 CAR positive cells from linear RNA (SEQ ID NO: 275) and circRNA (SEQ ID NO: 230).

FIG. 14F is a graph of anti-CD19 CAR expression in spleen on day 1, day 2 and day 3 comparing expression from linear mRNA and circRNA.

FIG. 14G is a plot showing area under the curve (AUC) for mGreenLantern (mGL) reporter protein expression by circRNAs comprising an exemplary IRES and circRNA comprising a reference IRES sequence (“circular RNA control”), compared to a linear RNA encoding mGL reporter protein in activated primary human T cells (from one donor).

FIG. 14H is a graph that showed a time course of anti-CD19 CAR+ T cells measured over 7 days in mice administered 1 mg/kg of circRNA encoding anti-CD19 CAR formulated in a targeted LNP (comprising Compound 7), and demonstrated that anti-CD19 CAR+ T cell populations were sustained in vivo.

FIG. 15A is a graph of the percentage of anti-CD19 CAR positive cells relative to total CD3 positive T cells in the blood of humanized mice, 24 hours after administration of each of three doses of RNA-LNP relative to vehicle control.

FIG. 15B is a graph of the percentage of anti-CD19 CAR positive cells relative to total CD3 positive T cells in the spleen of humanized mice, 24 hours after administration of each of three doses of RNA-LNP relative to vehicle control.

FIG. 15C is a graph of the percentage of B cells (relative to vehicle control) in blood after administration of three doses each of 0.5 mg/kg of RNA-LNP in mice, as measured 24 hours after administration of each dose, on day 1, 8 and 15.

FIG. 15D is a graph of the percentage of B cells (relative to vehicle control) in spleen after administration of three doses each of 0.5 mg/kg of RNA-LNP in mice, as measured 24 hours after administration of each dose, on day 1, 8 and 15.

FIG. 15E is a graph of the percentage of CD69-positive CD4+ T cells in blood 24 hours after administration of each of three doses of RNA-LNP.

FIG. 15F is a graph of the percentage of CD69-positive CD8+ T cells in blood 24 hours after administration of each of three doses of RNA-LNP.

FIG. 15G is a graph of the percentage of CD69-positive CD4+ T cells in spleen 24 hours after administration of each of three doses of RNA-LNP.

FIG. 15H is a graph of the percentage of CD69-positive CD8+ T cells in spleen 24 hours after administration of each of three doses of RNA-LNP. The results in FIG. 15E-FIG. 15H showed T cell activation in blood and spleen after RNA-LNP administration.

FIG. 16A is a graph of the percentage of anti-CD19 CAR positive cells as a percentage of total CD3 positive T cells in blood. The left panel shows anti-CD19 CAR positive cells, 24 hours after administration of 0.1 mg/kg, 0.5 mg/kg and 1.0 mg/kg doses of circRNA encoding anti-CD19 CAR relative to vehicle control. The right panel shows anti-CD19 CAR positive cells, 24 hours after administration of 0.5 mg/kg dose of circRNA encoding anti-CD19 CAR relative to vehicle control on days 1, 2 and 7 after administration.

FIG. 16B is a graph of the percentage of anti-CD19 CAR positive cells as a percentage of total CD3 positive T cells in spleen. The left panel shows anti-CD19 CAR positive cells, 24 hours after administration of 0.1 mg/kg, 0.5 mg/kg and 1.0 mg/kg doses of circRNA (encoding anti-CD19 CAR) relative to vehicle control. The right panel shows anti-CD19 CAR positive cells, 24 hours after administration of 0.5 mg/kg dose of RNA-LNP relative to vehicle control on days 1, 2 and 7 after administration.

FIG. 16C is a graph of the percentage of remaining B cells (relative to vehicle control) in blood. The left panel shows percent remaining B cells, 24 hours after administration of 0.1 mg/kg, 0.5 mg/kg and 1.0 mg/kg doses of RNA-LNP relative to vehicle control. The right panel shows percent remaining B cells, 24 hours after administration of 0.5 mg/kg dose of RNA-LNP relative to vehicle control on days 1, 2 and 7 after administration.

FIG. 16D is a graph of the percentage of remaining B cells (relative to vehicle control) in spleen. The left panel shows percent remaining B cells, 24 hours after administration of 0.1 mg/kg, 0.5 mg/kg and 1.0 mg/kg doses of RNA-LNP relative to vehicle control. The right panel shows percent remaining B cells, 24 hours after administration of 0.5 mg/kg dose of RNA-LNP relative to vehicle control on days 1, 2 and 7 after administration.

FIG. 17A is a graph of the percentage of anti-CD19 CAR positive cells as a percentage of total CD3 positive T cells in blood. The left panel shows anti-CD19 CAR positive cells, 24 hours after administration of 0.1 mg/kg, 0.5 mg/kg and 1.0 mg/kg doses of RNA-LNP relative to vehicle control. The right panel shows anti-CD19 CAR positive cells, 24 hours after administration of 0.5 mg/kg dose of RNA-LNP relative to vehicle control on days 1, 2 and 7 after administration.

FIG. 17B is a graph of the percentage of anti-CD19 CAR positive cells as a percentage of total CD3 positive T cells in spleen. The left panel shows anti-CD19 CAR positive cells, 24 hours after administration of 0.1 mg/kg, 0.5 mg/kg and 1.0 mg/kg doses of RNA-LNP relative to vehicle control. The right panel shows anti-CD19 CAR positive cells, 24 hours after administration of 0.5 mg/kg dose of circRNA (encoding anti-CD19 CAR) relative to vehicle control on days 1, 2 and 7 after administration.

FIG. 17C is a graph of the percentage of remaining B cells (relative to vehicle control) in blood. The left panel shows percent remaining B cells, 24 hours after administration of 0.1 mg/kg, 0.5 mg/kg and 1.0 mg/kg doses of circRNA (encoding anti-CD19 CAR) relative to vehicle control. The right panel shows percent remaining B cells, 24 hours after administration of 0.5 mg/kg dose of circRNA (encoding anti-CD19 CAR) relative to vehicle control on days 1, 2 and 7 after administration.

FIG. 17D is a graph of the percentage of B cells (relative to vehicle control) in spleen. The left panel shows percent remaining B cells, 24 hours after administration of 0.1 mg/kg, 0.5 mg/kg and 1.0 mg/kg doses of RNA-LNP relative to vehicle control. The right panel shows percent remaining B cells, 24 hours after administration of 0.5 mg/kg dose of RNA-LNP relative to vehicle control on days 1, 2 and 7 after administration.

FIG. 17E is a bar graph shown as an alternate representation of the right panel of FIG. 17B. FIG. 17E shows anti-CD19 CAR positive cells (as a percentage of CD3+ T cells), 24 hours after administration of 0.5 mg/kg dose of circRNA (encoding anti-CD19 CAR) formulated in targeted LNP relative to vehicle control on days 1, 2 and 7 after administration.

FIG. 17F is a bar graph shown as an alternate representation of FIG. 17D. FIG. 17F shows percent remaining B cells (relative to vehicle) after B cell count was normalized to total live splenocytes, 24 hours after administration of 0.5 mg/kg dose of circRNA (encoding anti-CD19 CAR) formulated in targeted LNP relative to vehicle control on days 1, 2 and 7 after administration.

FIG. 18 depicts immunohistochemistry staining in the left panel that shows targeted circRNA delivery to the white pulp in the spleen, where T cells are located. The right panel shows a schematic of the anatomical sites within the spleen, such as white pulp, marginal zone, red pulp, and B cell follicle. An expanded view in the right panel shows T cells localized within the white pulp of the spleen.

FIG. 19A is a graph of the percentage of B cells remaining (compared to vehicle), after administration of three doses at 1 mg/kg circRNA (encoding anti-CD19 CAR) in CD34⁺ NSG mice.

FIG. 19B is an image that showed immunohistochemistry staining of B cells using a CD20 marker in the spleen of CD34⁺ NSG mice administered a vehicle control depicting high density of B cells.

FIG. 19C is an image that showed immunohistochemistry staining of B cells using a CD20 marker in the spleen of CD34⁺ NSG mice of CD34⁺ NSG mice after administration of circRNA (encoding anti-CD19 CAR), which depicted B cell depletion.

FIG. 20A is a graph of the percentage of anti-CD20 CAR positive cells as a percentage of CD3+ T cells in blood from cynomolgus monkeys. The panel shows a time course of CAR expression after each dose of 1 mg/kg, 2 mg/kg, or 3 mg/kg circRNA relative to a vehicle control.

FIG. 20B is a graph of the number of anti-CD20 CAR-positive CD3+ T cells present in each microliter (μL) of blood. The panel shows the number of CAR+ T cells generated and present in blood at each time depicted in the graph. Each cynomolgus monkey was dosed on day 0 and re-dosed on day 7. The number of CAR+CD3+ T cells was measured by flow cytometry at baseline (e.g., 7 days prior to the dose administration), 6 hours, 24 hours after each dose, as well as 48 hours after the first dose.

FIG. 20C is a graph of the percentage of anti-CD20 CAR-positive CD3+ T cells present in the deep tissues (iliac lymph node and spleen). Each cynomolgus monkey was dosed on day 0 and day 7 at the 2 mg/kg and 3 mg/kg doses. The percentage of CAR+CD3+ T cells was measured by flow cytometry at 24 hours after the second dose. The graphs represent the average of two cynomolgus monkeys per dose.

FIG. 20D is a graph of the percentage of remaining B cells in blood over time relative to a vehicle control. Each cynomolgus monkey in Cohort 1 was dosed on day 0 and day 7 at the 1 mg/kg, 2 mg/kg, or 3 mg/kg doses. The graphs represent the average of two cynomolgus monkeys per dose. The absolute count of B cells present in circulation at each time point was measured by flow cytometry and used to calculate the remaining B cells relative to the number of B cells relative to the vehicle control.

FIG. 20E is a graph of the percentage of remaining B cells in deep tissue (iliac lymph node and spleen) relative to a vehicle control. Each cynomolgus monkey was dosed on day 0 and day 7 at 1 mg/kg, 2 mg/kg, or 3 mg/kg doses. For the 1 mg/kg group, the panel shows the percentage of B cells present in the tissues at 8 days after the second dose. For the 2 mg/kg and 3 mg/kg groups, the panel shows the percentage of B cells present in the tissues at 24 hours after the second dose. Each data point indicates a different animal.

FIG. 20F is a graph of the percentage of anti-CD20 CAR-positive cells as a percentage of CD3+ T cells in blood from cynomolgus monkeys in Cohort 2. The panel shows a time course of CAR expression from both single-dose and double-dose groups. For the single-dose group, the monkeys were administered 0.75 mg/kg or 1 mg/kg doses, and CAR expression was measured at 6 hours, 24 hours, and 48 hours after the dose. For the double-dose groups, the monkeys were administered 0.25 mg/kg, 0.5 mg/kg, 0.75 mg/kg, or 1 mg/kg doses twice (on day 0 and day 7). CAR expression was measured at 6 hours, 24 hours after each dose, and after 48 hours as well as after five days for the single-dose groups.

FIG. 20G is a graph of the number of anti-CD20 CAR-positive CD3+ T cells present in each microliter (uL) of blood. The panel shows the number of CAR+ T cells generated and present in blood at each time point depicted in the graph. The number of CAR+CD3+ T cells was measured by flow cytometry at baseline prior to the dose, at 6 hours, and 24 hours after each dose as well as 48 hours after the first dose.

FIG. 20H is a graph of the percentage of remaining B cells in the blood relative to a vehicle control. The panel shows a time course of B cell depletion for both single-dose and double-dose groups. For the single dose, the monkeys were administered circRNA (encoding anti-CD20 CAR) at 0.75 mg/kg or 1 mg/kg, and CAR expression was measured at 6 hours, 24 hours, and 48 hours after the dose. For the double-dose groups, the monkeys were administered twice at 0.25 mg/kg, 0.5 mg/kg, 0.75 mg/kg, or 1 mg/kg on day 0 and day 7. The number of B cells present in the blood was measured at 6 hours, 24 hours, and 48 hours after the first dose as well as five days later (120 hours) for the single-dose groups. In addition, the number of B cells was measured at 6 hours and 24 hours after the second dose.

FIG. 20I is a graph that shows the time course for the 0.25 mg/kg group in FIG. 20H.

FIG. 20J is a graph that shows the time course for the 0.5 mg/kg group in FIG. 20H.

FIG. 20K is a graph that shows the time course for the 0.75 mg/kg group in FIG. 20H.

FIG. 20L is a graph of the percentage of remaining B cells in the spleen relative to a vehicle control. For the single-dose groups, the monkeys were administered at 0.75 mg/kg or 1 mg/kg doses, and B cell depletion in the spleen was measured at day 5 after the dose. For the double-dose groups, the monkeys were administered twice each at 0.25 mg/kg, 0.5 mg/kg, 0.75 mg/kg, or 1 mg/kg on day 0 and day 7. On day 8, 24 hours after the second dose, the animals were sacrificed to quantify the number of B cells present in the tissue. B cell numbers were used to calculate the percentage of remaining B cell population relative to a vehicle control.

FIG. 20M is a graph of the percentage of remaining B cells in the iliac lymph node relative to a vehicle control. For the single-dose groups, the monkeys were administered 0.75 mg/kg or 1 mg/kg circRNA, and B cell depletion in the lymph node was measured at day 5 after the dose. For the double-dose groups, the monkeys were administered circRNA twice at 0.25 mg/kg, 0.5 mg/kg, 0.75 mg/kg, or 1 mg/kg each on day 0 and day 7. On day 8, 24 hours after the second dose, the monkeys were sacrificed to quantify the number of B cells present in the tissue. B cell numbers were used to calculate the % remaining B cell population relative to a vehicle control.

FIG. 20N is a graph of exemplary data from Cohorts 1 and 2 in the non-human primate study showing a liver enzyme (alanine aminotransferase, ALT) time course in blood following repeat administration of circRNA at day 0 and day 7 at doses of 0 mg/kg (vehicle control), 0.25 mg/kg, 0.5 mg/kg, 0.75 mg/kg, or 1.0 mg/kg each. This graph showed safety and tolerability of the circRNA in monkeys.

FIG. 20O is a graph of the percentage of remaining CD20+ B cells in the lymph node relative to a vehicle control in monkeys that were administered 0.25 mg/kg, 0.5 mg/kg, or 0.75 mg/kg circRNA twice on day 0 and day 7, and subsequently B cell depletion in the lymph node was measured 24 hours after the second dose (that is, on day 8). Viable lymphocytes isolated from monkeys were gated using a CD20 marker for quantifying a CD20+ B cell subpopulation by flow cytometry, and CD20+ B cell numbers were used to calculate the percentage of remaining CD20+ B cells relative to a vehicle control.

FIG. 20P is a graph showing the percentage of remaining CD20+ B cells in the spleen relative to a vehicle control (bottom panel). As part of the study, the monkeys in Cohort 1 were administered circRNA encoding anti-CD20 CAR at a single dose of 0.75 mg/kg or 1 mg/kg, and B cell depletion in the spleen was measured at day 5 after the dose (as illustrated in the top panel). CD20+ gated B cell numbers were used to calculate the percentage of remaining CD20+ B cells relative to a vehicle control.

FIG. 20Q is a graph showing the percentage of remaining CD20+ B cells in the spleen relative to a vehicle control. The monkeys (in Cohorts 1 and 2) were administered circRNA encoding anti-CD20 CAR twice each at 0.25 mg/kg, 0.5 mg/kg, 0.75 mg/kg, 1 mg/kg, 2 mg/kg or 3 mg/kg on day 0 and day 7. On day 8, 24 hours after the second dose, the animals were sacrificed to quantify the number of B cells present in the tissue. CD20+ gated B cell numbers were used to calculate the percentage of remaining CD20+ B cell population relative to a vehicle control. The graph combines the NHP data from Cohort 2 (for the 0.25 mg/kg, 0.5 mg/kg, 0.75 mg/kg, and 1 mg/kg doses) and Cohort 1 (for the 2 mg/kg and 3 mg/kg doses).

FIG. 20R depicts in situ hybridization (ISH) images that were generated 24 hours after the second dose of circRNA was administered to the monkeys. The ISH images showed that after two doses of circRNA, at 0.5 mg/kg or higher, formulated in targeted LNP, full depletion of B cells was seen in the spleen. The CD20 marker stained B cells, and DAPI stained cell nuclei.

FIG. 20S is a graph showing the percentage of remaining CD20+ B cells in the lymph node relative to a vehicle control (bottom panel). As part of the study, the monkeys were administered circRNA encoding anti-CD20 CAR as a single dose, and B cell depletion in the lymph node was measured at day 5 after the dose (as illustrated in the top panel). Representative data from the 0.75 mg/kg or 1 mg/kg doses are shown. CD20+ gated B cell numbers were used to calculate the percentage of remaining CD20+ B cells relative to a vehicle control.

FIG. 20T is a graph showing the percentage of remaining CD20+ B cells in the lymph node relative to a vehicle control. The monkeys (Cohorts 1 and 2) were administered circRNA encoding anti-CD20 CAR twice, on day 0 and day 7, each dose at 0.25 mg/kg, 0.5 mg/kg, 0.75 mg/kg, 1 mg/kg, 2 mg/kg or 3 mg/kg. On day 8, 24 hours after the second dose, the animals were sacrificed to quantify the number of B cells present in the tissue. CD20+ gated B cell numbers were used to calculate the percentage of remaining CD20+ B cells relative to a vehicle control. The graph combines the data from Cohort 2 (for the 0.25 mg/kg, 0.5 mg/kg, 0.75 mg/kg, and 1 mg/kg doses) and Cohort 1 (for the 2 mg/kg and 3 mg/kg doses).

FIG. 20U depicts ISH images in the lymph node that showed that after two doses of the circRNA encoding anti-CD20 CAR formulated in targeted LNP was administered to the cynomolgus monkeys, robust B cell depletion was observed at day 8, 24 hours after the second dose. The CD20 marker stained B cells, and DAPI stained cell nuclei.

FIG. 20V shows magnified versions of the ISH image for the 0.75 mg/kg dose from FIG. 20U. As shown in FIG. 20V (top right panel) although remaining B cells were detected, those B cells (CD20 stained) co-localized with the presence of circRNA (anti-CD20 CAR circRNA stained). Although not bound by any mechanism of action, the detection of the circRNA suggested that T cells transfected with the circRNA were in the process of depleting B cells, that is, that there was an ongoing B cell depletion phenomenon.

FIG. 21A depicts ISH images of the spleen from day 1, day 5, and day 28 after a single administration of 1 mg/kg of the circRNA encoding anti-CD20 CAR formulated in targeted LNP to cynomolgus monkeys. The ISH images show T cells (stained with CD3 marker) and B cells (stained with CD20 marker) at day 1, day 5, and day 28 after administration of the circRNA, as well as after administration with a vehicle control at day 28.

FIG. 21B depicts ISH images of the lymph node from day 1, day 5, and day 28 after a single administration of 1 mg/kg of the circRNA encoding anti-CD20 CAR formulated in targeted LNP to cynomolgus monkeys. The ISH images show T cells (stained with CD3 marker) and B cells (stained with CD20 marker) at day 1, day 5, and day 28 after administration of the circRNA, as well as after administration with a vehicle control at day 28.

### DEFINITIONS

In order for the present disclosure to be more readily understood, certain terms are first defined below. Additional definitions for the following terms and other terms are set forth throughout the specification.

About or approximately: As used herein, the term “about” or “approximately,” as applied to one or more values of interest, refers to a value that is similar to a stated reference value. In certain embodiments, the term “approximately” or “about” is understood as within a range of normal tolerance in the art, for example, within two standard deviations of the mean, and refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term refers to a range of values that fall within 10% of the stated reference value. In some embodiments, the term refers to a range of values that fall within 5% of the stated refer. The term “between” includes the values of the specified boundaries and all intervening values and fractions.

Adaptive immune response: The adaptive immune response is typically understood to be antigen-specific. Antigen specificity allows for the generation of responses that are tailored to specific antigens, pathogens or pathogen-infected cells. The ability to mount these tailored responses is maintained in the body by “memory cells”. Should a pathogen infect the body more than once, these specific memory cells are used to quickly eliminate it. In this context, the first step of an adaptive immune response is the activation of naïve antigen-specific T cells or different immune cells able to induce an antigen-specific immune response by antigen-presenting cells. This occurs in the lymphoid tissues and organs through which naïve T cells are constantly passing. Cell types that can serve as antigen-presenting cells are inter alia dendritic cells, macrophages, and B cells. Each of these cells has a distinct function in eliciting immune responses. Dendritic cells take up antigens by phagocytosis and macropinocytosis and are stimulated by contact with e.g., a foreign antigen to migrate to the local lymphoid tissue, where they differentiate into mature dendritic cells. Macrophages ingest particulate antigens such as bacteria and are induced by infectious agents or other appropriate stimuli to express major histocompatibility complex (MHC) molecules. The unique ability of B cells to bind and internalize soluble protein antigens via their receptors may also be important to induce T cells. Presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which do not recognize and bind antigen directly, but instead recognize short peptide fragments e.g., of pathogen-derived protein antigens, which are bound to MHC molecules on the surfaces of other cells.

Adenylation: As used herein, “adenylation” or “AMPylation” is a process in which an adenosine monophosphate (AMP) molecule is covalently attached to the amino acid side chain of a protein. This covalent addition of AMP to a hydroxyl side chain of the protein is a posttranslational modification.

Adjuvant: the term “adjuvant” refers to agents which confer immunity by themselves. An adjuvant assists the immune system un-specifically to enhance the antigen-specific immune response by e.g., promoting presentation of an antigen to the immune system or induction of an unspecific innate immune response. Furthermore, an adjuvant may preferably, e.g., modulate the antigen-specific immune response by e.g., shifting the dominating Th2-based antigen specific response to a more Th1-based antigen specific response or vice versa. Accordingly, an adjuvant may favorably modulate cytokine expression/secretion, antigen presentation, type of immune response etc. An adjuvant or an adjuvant component in the broadest sense is typically a (e.g., pharmacological or immunological) agent or composition that may modify, e.g., enhance, the efficacy of other agents, such as a drug or vaccine. Conventionally the term refers in the context of the invention to a compound or composition that serves as a carrier or auxiliary substance for immunogens and/or other pharmaceutically active compounds. It is to be interpreted in a broad sense and refers to a broad spectrum of substances that are able to increase the immunogenicity of antigens incorporated into or co-administered with an adjuvant in question. In the context of the present invention an adjuvant will preferably enhance the specific immunogenic effect of the active agents of the present invention. Typically, “adjuvant” or “adjuvant component” has the same meaning and can be used interchangeably.

Antibody: As used herein, the term “antibody” is referred to in the broadest sense and specifically covers various embodiments including, but not limited to monoclonal antibodies, polyclonal antibodies, multi-specific antibodies (e.g., bispecific antibodies formed from at least two intact antibodies), and includes antibody fragments (e.g., diabodies) so long as they exhibit a desired biological activity (e.g., “functional”). Antibodies are primarily amino acid based molecules but may also comprise one or more modifications (including, but not limited to the addition of sugar moieties, fluorescent moieties, chemical tags, etc.). Non-limiting examples of antibodies or fragments thereof include VH and VL domains, scFvs, Fab, Fab′, F(ab′)2, Fv fragment, diabodies, nanobodies, linear antibodies, single chain antibody molecules, multi-specific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

Antibody fragment or antigen-binding fragment: As used herein, “antibody fragment” or “antigen-binding fragment” refers to any portion of an intact antibody. In some embodiments, antibody fragments comprise antigen-binding regions from intact antibodies. Examples of antibodies or antigen binding fragments may include, but are not limited to Fab, Fab′, F(ab′)₂, Fv fragments; diabodies; single domain antibodies (sdAb) (e.g., variable domain of heavy chain only (VHH) antibodies (also referred to as nanobodies) or Variable New Antigen Receptor (VNAR) antibodies); linear antibodies; single-chain antibody molecules (e.g., scFv); and multi-specific antibodies formed from antibody fragments. In some embodiments, the antibody or antigen-binding fragment is a VHH or an scFv. In some embodiments, the antibody or antigen-binding fragment is a VHH. In some embodiments, the antibody or antigen-binding fragment is an scFv.

Antibody variant: As used herein, “antibody variant” refers to a biomolecule resembling an antibody in structure and/or function comprising some differences in their amino acid sequence, composition or structure as compared to a native antibody.

Antigen: The term “antigen” refers to a substance which may be recognized by the immune system, preferably by the adaptive immune system, and is capable of triggering an antigen-specific immune response, e.g., by formation of antibodies and/or antigen-specific T cells as part of an adaptive immune response. Typically, an antigen may be or may comprise a peptide or protein which may be presented by the MHC to T-cells. In the sense of the present invention an antigen may be the product of translation of a provided nucleic acid molecule, preferably an RNA as defined herein. In this context, fragments, variants and derivatives of peptides and proteins comprising at least one epitope are understood as antigen.

Associated: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also be through ionic or hydrogen bonding or a hybridization-based connectivity sufficiently stable such that the “associated” entities remain physically associated.

Biologic: As used herein, a “biologic” is a polypeptide-based molecule encoded by the linear and/or circular RNA provided herein which may be used to treat, cure, mitigate, prevent, or diagnose a disease or disorder.

Cellular immunity or cellular immune response: the phrase “cellular immunity”, which is used interchangeably with “cellular immune response”, relates typically to the activation of macrophages, natural killer cells (NK), antigen-specific cytotoxic T lymphocytes, and the release of various cytokines in response to an antigen. In a more general way, cellular immunity is not related to antibodies but to the activation of cells of the immune system. A cellular immune response is characterized e.g. by activating antigen-specific cytotoxic T lymphocytes that are able to induce apoptosis in body cells displaying epitopes of an antigen on their surface, such as virus-infected cells, cells with intracellular bacteria, and cancer cells displaying tumor antigens; activating macrophages and natural killer cells, enabling them to destroy pathogens; and stimulating cells to secrete a variety of cytokines that influence the function of other cells involved in adaptive immune responses and innate immune responses.

Circular RNA: As used herein, the term “circular RNA” or “circRNA” refers to an RNA that forms a circular structure through covalent or non-covalent bonds. The terms “circRNA” or “circular polyribonucleotide” or “circular RNA” are used interchangeably. In some embodiments, circRNAs are covalently closed, single stranded RNA molecules. A circular RNA can be produced by various methods, including back-splicing of a linear precursor RNA, by chemical ligation and/or enzymatic ligation. Circular RNAs (circRNAs) can be endogenous or synthetic. Synthetically created and exogenously delivered circRNAs can be synthesized in vitro using self-splicing permuted introns (e.g., self-splicing Group I or Group II intron) from in vitro transcribed constructs. Unlike linear RNAs, circular RNAs are more resistant to the degradation by exonuclease and have a longer half-life than their corresponding linear counterparts. A circular RNA can be a circular RNA that encodes a polypeptide or synthetic protein (e.g., a chimeric antigen receptor). In some embodiments, circular RNAs disclosed herein have significantly improved drug-like properties compared to linear mRNA therapeutics, including enhanced longevity as protein production vectors.

Conjugation: As used herein, the term “conjugation” refers to a process that creates a linkage between two moieties. In some embodiments, the conjugation is a chemical reaction. In some embodiments, the conjugation is an enzymatic, or enzyme-catalyzed, reaction.

Conjugation Handle: As used herein, the term “conjugation handle” refers to a short peptide containing one or more amino acids (e.g., 1-20 amino acids). In some embodiments, the conjugation handle includes at least one reactive group (e.g., azide, tetrazine, or thiol) that allows conjugation between two moieties via, e.g., chemical, enzymatic, or enzyme-catalyzed reaction. The reactive group in the conjugation handle may be a functional group of an amino acid on the “conjugation handle” or attached anywhere on the “conjugation handle” (e.g., at the N-terminus, C-terminus, or internal to the conjugation handle) directly or indirectly. In some embodiments, the reactive group described herein is suitable for a “click chemistry” reaction. In some embodiments, the conjugation handle comprises one or more glycine, alanine, cysteine, serine, and/or glutamine. A “conjugation handle” may contain, for example, 5-10 amino acids, such as 5, 6, 7, 8, 9, or 10 amino acids. A “conjugation handle” may be attached to (e.g., covalently attached to) a moiety (such as a targeting moiety) that is conjugated to, or is to be conjugated to, another moiety, such as a delivery vehicle. In such cases, a conjugation handle may include a naturally-occurring or modified amino acid containing a reactive group. In embodiments, a conjugation handle may be attached to a moiety (such as the targeting moiety), directly (e.g., by covalent attachment) or indirectly. As used herein, in some embodiments, “conjugation handle” also refers to a short peptide that is part of a conjugation product formed by joining two moieties. For instance, the conjugation handle may be joined to a targeting moiety and become part of a longer sequence, e.g., the conjugation handle may be attached to the sequence of a targeting moiety that comprises or consists of an antibody or antigen-binding fragment (e.g., a VHH). In some embodiments, the conjugation handle is attached to the C terminus of the antibody or antigen-binding fragment.

Corresponding to: As used herein, the term “corresponding to” refers to a nucleic acid sequence or an amino acid sequence at particular positions of an intron, or the corresponding positions in another intron. A sequence corresponding to the sequence at particular positions of an intron may comprise a corresponding substitution or a variant, e.g., the substituted nucleotides or amino acids do not naturally occur at the corresponding positions. The substituted nucleotides or amino acids may be the corresponding residues in another intron (e.g., Group I or II intron).

Delivery: As used herein, “delivery” refers to the act or manner of delivering a compound, substance, entity, moiety, cargo or payload.

DNA Nuclease: As used herein, “DNA nuclease” is an enzyme that catalyzes the cleavage of phosphodiester bonds. DNA nucleases play a role in DNA replication and various DNA repair processes, base excision repair, nucleotide excision repair, mismatch repair, and double strand break repair. Depending on whether a 5′ or 3′ end is required for substrate recognition and whether cleavage products are single or oligonucleotides, DNA nucleases are classified as exonucleases and endonucleases. For example, self-cleaving ribozymes cleave RNAs endonucleolytically, while exonucleases cleave one nucleotide at a time from one end, either from 5′ to 3′ or from 3′ to 5′. Some nucleases have both exonuclease and endonuclease activities, for example, Flap endonuclease 1 (FEN1) has 5′ to 3′ exonuclease activity in addition to endonuclease activity, and Mre11 has both endonuclease and 3′ to 5′ exonuclease activities.

DNase I: As used herein, “DNase I” or “deoxyribonuclease I” refers to enzymes that cleave single or double-stranded DNA and require divalent metal ions to hydrolyze DNA yielding 3′-hydroxyl and 5′-phosphorylated products.

Downstream: As used herein, the term “downstream” refers to sequence that is 3′ to a particular sequence.

Encapsulate: As used herein, the term “encapsulate” means to enclose, surround, or encase. As it relates to the formulation of the compositions of the disclosure, encapsulation may be substantial, complete or partial. The term “substantially encapsulated” means that at least greater than 50%, 60%, 70%, 80%, 85%, 90%, 95%, 96%, 97%, 98%, 99%, 99.9%, 99.9% or greater than 99.999% of the pharmaceutical composition of the disclosure may be enclosed, surrounded or encased within the delivery agent. “Partially encapsulated” means that less than 10%, 20%, 30%, 40%, 50%, or less of the pharmaceutical composition or compound of the disclosure may be enclosed, surrounded or encased within the delivery agent. In some embodiments, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of the pharmaceutical composition of the present disclosure are encapsulated in the delivery vehicle (e.g., a lipid nanoparticle (LNP)).

Encode: As used herein, the term “encode” or “encoding” refers broadly to any process whereby the information in a polymeric macromolecule is used to direct the production of a second molecule that is different from the first. The second molecule may have a chemical structure that is different from the chemical nature of the first molecule. For example, DNA encodes RNA and RNA encodes a polypeptide or protein.

Enhance: As used herein, the terms “enhance” and “enhancement” refers to an increase of at least about 5%, 10%, 20%, 25%, 50%, 75%, 100%, 150%, 200%, 300%, 400%, 500% or more of a reference; the reference may be a biological function of a nucleic acid or protein and a gene expression level, etc.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing) (RNA expression); (3) translation of an RNA into a polypeptide or protein; and (4) post-translational modification of a polypeptide or protein (Protein expression).

Feature: As used herein, a “feature” refers to a characteristic, a property, or a distinctive element. Features may refer to features of polynucleotides (e.g., circular polynucleotide of the present disclosure) such as sequence-based (nucleotides, modified nucleotides) or structural features (loops, folds, stem-loops, stems, hairpins, bulges, half-loops, etc.). Features may also refer to features of the polypeptides encoded by the present circular polynucleotide, such as surface manifestations, local conformational shape, folds, loops, half-loops, domains, half-domains, sites, termini or any combination thereof.

Formulation: As used herein, a “formulation” includes one or more compound, substance, entity, moiety (e.g., targeting moiety), cargo, or payload (such as RNA) and a delivery vehicle (e.g., lipid nanoparticle or virus like particles).

Fragment: A “fragment,” as used herein, refers to a portion. For example, an intron fragment may comprise a portion of the full intron sequence. Fragments of proteins may comprise polypeptides obtained by digesting full-length protein isolated from cultured cells.

Gap: As used therein, the term “gap” refers to a small region with the DNA splint comprising a stretch of nucleotides that are not complementary to any sequence of linear RNAs that are to be circularized. The gap region can be in the middle nucleotides of the splint, creating a situation where the splint ‘gap’ is single stranded, and the terminal 3′ and 5′ ends of the RNA molecule are also single stranded (depending on size of the gap).

Homology: As used herein, the term “homology” refers to the overall relatedness between polymeric molecules, e.g., between polynucleotide molecules (e.g., DNA molecules and/or RNA molecules). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two polynucleotide sequences. In accordance with the disclosure, two polynucleotide sequences are considered to be homologous if the polypeptides they encode are at least about 50%, 60%, 70%, 80%, 90%, 95%, or even 99% identical for at least one stretch of at least about 20 amino acids. In some embodiments, homologous polynucleotide sequences are characterized by the ability to encode a stretch of at least 4-5 uniquely specified amino acids. For example, in some embodiments, for polynucleotide sequences less than 60 nucleotides in length, homology is determined by the ability to encode a stretch of at least 4-5 uniquely specified amino acids.

Hydropathy Index: As used herein, the term “hydropathy index” is a quantitative measure that refers to the hydrophilicity and hydrophobicity of a nucleobase sequence (e.g., an RNA sequence) by combining (i) experimentally measured nucleobase partition coefficients (log P) measuring distribution of the nucleobase between an immiscible organic solvent (e.g., n-Octanol) and water and (ii) normalization to length of the sequence, enabling comparison across sequences of different lengths. Typically, a more negative log P indicates a more hydrophilic nucleobase, while a less negative or positive log P indicates a more hydrophobic nucleobase. Experimental nucleobase log P values have been experimentally determined for canonical nucleobases and are as follows: cytosine: −1.7 (most hydrophilic), uracil: −1.1 (moderate hydrophilicity), guanine: −0.9 (moderate hydrophilicity) and adenine (least hydrophilic). (Zamora W J et al., J. Phys. Chem. B, 2017, 13, 10 908-10922). Poly A sequences, which have a higher hydropathy index, increasingly associate with ionizable-lipid-rich regions at the LNP membrane surface remaining in contact with the membrane rather than remaining freely dispersed within the LNP. This heightened tendency to interact is undesirable, as it reflects stronger RNA-lipid association that can impede efficient delivery. In contrast, a sequence with a lower hydropathy index would be less likely to interact with ionizable lipids and thus support improved cellular uptake and cytoplasmic release. (Gilbert et al. 2024, Nanoscale, 2024, 16, 777-794). Protocol for Assigning a Residue's Character on the Hydropathy Scale (PARCH) analysis has confirmed that base composition is the primary determinant of hydropathy as the phosphate-ribose backbone is uniformly highly hydrophilic and does not vary between sequences. Hydropathy of RNA sequences in the current disclosure is calculated by computing an additive hydropathy sum Hraw=Σᵢ(nᵢ*log Pᵢ) where nᵢ is the number of nucleobases of each type, e.g., n_{A}, n_{C}, n_{G}, n_{U} and log P values are as determined by Zamora et al. Finally, the Hraw values are normalized to length of the RNA sequence so that hydropathy indices of sequences of different lengths may be directly compared. i.e.,

$$Hydropathy index = \frac{{H}_{raw}}{L} .$$

Further, normalizing to length provides a per nucleotide average hydrophilicity. As used herein, the hydropathy index refers to intrinsic hydropathy of the primary nucleic acid sequence without regard to folding, secondary structure or tertiary interactions.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, considering the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Immune response: The term “immune response” includes both a specific reaction of the adaptive immune system to a particular antigen (so called “specific” or “adaptive” immune response), and an unspecific reaction of the innate immune system (so called “unspecific” or “innate” immune response). In some aspects, the invention relates to eliciting specific reactions (adaptive immune responses) of the adaptive immune system. Furthermore, the invention also relates to eliciting an innate immune response, since the specific (adaptive) immune response can be supported by an additional unspecific reaction (innate immune response). Therefore, in some aspects, the invention also relates to simultaneous stimulation of the innate and the adaptive immune system to evoke an efficient immune response.

Innate immune response: The term “innate immune response”, also known as non-specific immune response, comprises the cells and mechanisms that defend the host from infection by other organisms in a non-specific manner. The cells of the innate system recognize and respond to pathogens in a generic way, but, unlike the adaptive immune system, the innate immune response does not confer long-lasting or protective immunity to the host. The innate immune system may be activated, for example, by ligands of pathogen-associated molecular patterns (PAMP) receptors, Toll-like receptors (TLRs), or other auxiliary substances such as lipopolysaccharides, TNF-alpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR10, TLR11, TLR12 or TLR13, a ligand of a NOD-like receptor, a ligand of a RIG-1 like receptor, an immunostimulatory nucleic acid, an immunostimulatory RNA (isRNA), an antibacterial agent, or an anti-viral agent. Typically, a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system through, a process known as antigen presentation; and/or acting as a physical and chemical barrier to infectious agents.

Internal Homology Element: As used herein, the terms “internal homology region,” “internal homology element,” and “inner homology element (IHE)” are used interchangeably. The internal homology region refers to complementary sequences at the 5′ end and the 3′ end of precursor RNA (or DNA template from which the precursor RNA is transcribed), referred to as the 5′ internal homology element and the 3′ internal homology element, respectively. The 5′ internal homology element forms intramolecular Watson-Crick base pairs with the 3′ internal homology element, such that the homology elements have at least 75% sequence complementarity. These base-pairing interactions stabilize 5′ end to 3′ end association and promote circularization of the RNA. The internal homology region ranges from 5-50 nucleotides in length, more typically, between 5-20 nucleotides in length.

Intron: As used herein, the term “intron” means any sequence within a gene that is removed by RNA splicing during maturation of RNA transcripts from pre-mRNA transcripts. The term “intron” also refers to any sequence within a precursor linear RNA that is spliced out during circularization to form circular RNA. Introns in general are non-coding sequences of an RNA transcript. “Intron” can also refer to intron fragments.

Ionizable Lipid: As used herein, “ionizable lipid” refers to any of several lipid species that carry a net positive charge at a selected pH. For example, and without limitation, an ionizable lipid may carry a net positive charge at an acidic pH (e.g., pH of about 4.0), but remain neutrally charged at a physiological pH. Ionizable lipids typically comprise an ionizable head group such as amine linked to a hydrophobic tail region comprising aliphatic chain of varying degree of saturation.

Linker: As used herein, “linker” refers to a short amino acid or polypeptide sequence that operably connects, joins or links two protein sequences, including without limitation, antibodies or domains, e.g., VH and VL. A linker can be fused to an N-terminus or a C-terminus of a polypeptide. A linker is of any length, for example, 3-100 amino acids long.

Lipid Nanoparticle: As used herein “lipid nanoparticle” or “LNP” refers to a delivery vehicle comprising one or more lipids (e.g., cationic lipids, non-cationic lipids, Modified-PEG-lipids). An “ionizable lipid” refers to any of several lipid species that carry a net positive charge at a selected pH. A “non-cationic lipid” refers to any neutral, zwitterionic or anionic lipid.

Liposome: As used herein, “liposome” generally refers to a vesicle composed of lipids (e.g., amphiphilic lipids) arranged in one or more spherical bilayers or bilayers.

microRNA or miRNA or miR: As used herein, “microRNA” or “miRNA” or “miR” refers to small non-coding ribonucleic acids that regulates gene expression. A “miRNA” or “microRNA” is a single-stranded RNA of short length (for example, 18-25 nucleotides) that functions not to encode proteins, but to regulate biological processes such as cell proliferation or differentiation, immune response, and expression of other genes at the post-transcriptional level. The dysregulation of microRNA may be involved in various diseases, including autoimmune diseases and cancer.

Micro RNA (miRNA) binding sequence or miRNA binding site: As used herein, the term “microRNA binding sequence,” “miRNA binding sequence”, “microRNA binding site,” or “miRNA binding site” refers to a binding sequence (e.g., a short nucleic acid fragment) that binds to a miRNA. In some embodiments, the circular polynucleotide, such as circular RNA, comprise one or more miRNA binding sites. The number of miRNA binding sites in the circular polynucleotide is variable and relates to the length of the circular polynucleotide. As non-limiting examples, the circular polynucleotide comprises 1, 2, 3, 4, 5, 6, 7, 8, 9, 10 or more miRNA binding sites. The multiple miRNA binding sites may have the same nucleic acid sequences and bind to the same miRNA; or alternatively, the miRNA binding sites have different nucleic acid sequences and bind to different miRNAs, such as 2, 3, 4, 5, or more different miRNAs.

Modified: As used herein “modified” or, as appropriate, “modification” refers to a changed state or structure of a molecule. Molecules may be modified in many ways including chemically, structurally, and functionally. With respect to nucleic acid molecules (e.g., DNA and RNA), the modifications are modified version of A, G, C, U or T nucleotides. With respect to polypeptides, the term “modification” refers to a modification to the canonical set of 20 amino acids.

mRNA: As used herein, the term “messenger RNA” (mRNA) means a polynucleotide which encodes a polypeptide of interest or synthetic polypeptide, and which is capable of being translated to produce the encoded polypeptide of interest or synthetic polypeptide, such as a CAR construct, in vitro, in vivo, in situ or ex vivo.

Nucleic acid molecule: As used herein, the term “nucleic acid molecule” refers to a polymeric form of nucleotides, either deoxyribonucleotides (DNAs) or ribonucleotides (RNAs), or analogs thereof. The terms include single stranded or double-stranded molecules comprised of nucleic acid bases. As such, the term includes, and may be used interchangeably with “plasmids”, “constructs”, or “vectors.” In the present disclosure, the terms “polynucleotide” and “nucleic acid” are used interchangeably. As is commonly understood, DNA is double-stranded. Any DNA sequences provided in the present disclosure may be sense or anti-sense sequences, and it is understood that the sequences of both DNA strands and the transcribed RNA product from either DNA strand are fully encompassed by the present disclosure. It is commonly understood that DNA includes nucleobases adenine, cytosine, thymine, and guanine, and RNA includes nucleobases adenine, cytosine, uracil, and guanine. Thymine in DNA is replaced by uracil in RNA; both thymine and uracil function as the complementary base to adenine. In the context of both a DNA and an RNA sequence, it is understood that A represents adenine, C represents cytosine, and G represents guanine. In the context of a DNA sequence, it is understood that T represents thymine. In the context of an RNA sequence, it is understood that U and T both represent uracil; throughout the present disclosure any reference to T in an RNA sequence means uracil.

Pharmaceutical composition: As used herein the term “pharmaceutical composition” refers to compositions comprising at least one active ingredient or drug substance and optionally one or more pharmaceutically acceptable excipients. The pharmaceutical composition can comprise a circRNA as disclosed herein or a circRNA therapeutic as disclosed herein. In some embodiments, the pharmaceutical composition comprises a circRNA as disclosed herein as the active ingredient or drug substance. In some embodiments, the pharmaceutical composition comprises a targeted LNP as disclosed herein as an exemplary excipient.

Polydispersity Index: As used herein, the term “polydispersity index” or “PDI” refers to a range of value between 0 to 1 that defines the homogeneity or heterogeneity of lipid nanoparticle population. PDI value close to 0 indicates homogeneity of lipid nanoparticle population, whereas PDI value close to 1 indicates heterogeneity of the population. In some embodiments, the polydispersity index (PDI) is about 0 to 0.1 (e.g., 0.00, 0.01, 0.02, 0.03, 0.04, 0.05, 0.06, 0.07, 0.08, 0.09 or 0.1).

Polypeptides: As used herein, the term “polypeptides” refer to any polypeptide, both synthetic and naturally occurring, encoded in the linear and/or circular RNA of the present invention. As used herein, “polypeptide” means a polymer of amino acid residues (natural or unnatural) linked together, most often by peptide bonds. In some embodiments, the term, as used herein, refers to proteins (i.e., proteins of interest), polypeptides, and peptides of any size, structure, or function. In some embodiments, the polypeptide encoded is smaller than about 50 amino acids and the polypeptide is then termed a peptide. For example, a peptide is at least about 2, 3, 4, or at least 5 amino acid residues long. In some embodiments, polypeptides include gene products, naturally occurring polypeptides, synthetic polypeptides, homologs, orthologs, paralogs, fragments and other equivalents, variants, and analogs of the foregoing. In some embodiments, a polypeptide is a single molecule. In some embodiments, a polypeptide includes a multi-molecular complex such as a dimer, trimer or tetramer. In some embodiments, a polypeptide comprises single chain or multichain polypeptides such as antibodies or insulin and is associated or linked. In some embodiments, disulfide linkages are found in multichain polypeptides. In some embodiments, the term polypeptide applies to amino acid polymers in which one or more amino acid residues are an artificial chemical analogue of a corresponding naturally occurring amino acid. In some embodiments, the polypeptide (e.g., synthetic polypeptide) is a chimeric antigen receptor.

Polypeptide variant: As used herein, “polypeptide variant” refers to molecules which differ in their amino acid sequence from a native, wild-type or reference sequence. In some embodiments, the amino acid sequence variants possess substitutions, deletions, and/or insertions at certain positions within the amino acid sequence, as compared to a native or reference sequence. Typically, variants possess at least about 50% identity (homology) to a native or reference sequence. In some embodiments, they possess at least about 80%, at least about 90% identity to a native, wild-type or reference sequence.

Ribosome recruiting element: As used herein, a “ribosome recruiting element” includes naturally occurring or engineered RNA sequences that directly or indirectly recruit the ribosome to initiate translation of RNA into protein. A ribosome recruiting element is an RNA sequence that recruits ribosomes to initiate translation of, for example, circRNA, in a cap-independent manner. In some embodiments, a ribosome recruiting element is an internal ribosome entry site (IRES). For circRNA, an IRES plays a critical role in translation initiation of the circRNA in the absence of a 5′ cap by directly recruiting ribosomes to an internal position of circRNA in a cap-independent manner to initiate translation. In other embodiments, a ribosome recruiting element is a branched cap structure (e.g., qRNA) added to circular RNA that initiates translation by the ribosome. In some embodiments, a ribosome recruiting element is an IRES transacting factor (ITAF) recruiting element. In some embodiments, IRES and ITAFs recruit the ribosome. In some embodiments, the ITAF is specific to an IRES or group of related IRES elements. In some embodiments, the ITAF is general (for example, polypyrimidine tract-binding protein, PTB).

Signal peptide: As used herein, a “signal sequence” or “signal peptide” is a polynucleotide or polypeptide, respectively, which is from about 9 to 200 nucleotides (3-60 amino acids) in length which is incorporated at the 5′ terminus of the coding region or the N-terminus polypeptide encoded, respectively. In some embodiments, addition of these sequences results in trafficking of the encoded polypeptide to the endoplasmic reticulum through one or more secretory pathways. Some signal peptides are cleaved from the protein by signal peptidase after the proteins are transported.

Spacer: As used herein, the term “spacer” refers to any contiguous nucleotide sequence (e.g., of one or more nucleotides) that provides distance or flexibility between two adjacent polynucleotide regions. The spacer can be a 5′ or 3′ spacer. In some embodiments, the nucleic acid for making a circular RNA (e.g., precursor RNA) of the present invention comprises a 5′ spacer that is located between the upstream intron fragment and a sequence of interest (i.e., a coding sequence to be included in an RNA to be circularized). In some embodiments, the 5′ spacer is between the upstream intron fragment and the IRES. In some embodiments, the nucleic acid for making a circular RNA of the present invention comprises a 3′ spacer that is located between a sequence of interest and the downstream intron fragment. The 5′ and 3′ spacer sequences may be 10 nucleotides to 100 nucleotides in length, or 20 nucleotides to 50 nucleotides in length. In some embodiments, the 5′ spacer is at least 10 nucleotides in length. In some embodiments, the 5′ spacer sequence is at least 15 nucleotides in length. In some embodiments, the 5′ spacer is at least 20 nucleotides in length. In some embodiments, the 5′ spacer sequence is at least 30 nucleotides in length. In some embodiments, a 3′ spacer is located between a sequence to be circularized and the downstream intron sequence. In the precursor RNA and the resultant circular RNA following circularization, within the 5′ UTR or the 3′ UTR, more typically, the 3′ UTR, a microRNA binding site is flanked by an upstream spacer and a downstream spacer. The upstream spacer refers to a contiguous non-coding sequence 5′ of the microRNA binding site. The downstream spacer refers to a contiguous non-coding sequence 3′ of the microRNA binding site. Typically, the upstream spacer and/or downstream spacer comprises between 12-18 nucleotides and are of comparable length. In the present disclosure, the upstream spacer and/or downstream spacer comprise no more than five contiguous adenine sequences (for example, two, three, four or five contiguous adenine sequences are interrupted by a cytosine).

Structural Lipid: As used herein, “structural lipid” refers to sterols and lipids containing sterol moieties.

Subject: As used herein, the terms “subject” and “patient” may be used interchangeably herein. As such, unless otherwise indicated, a “subject” refers to a human that is being treated for a disease, or prevention of a disease, such as a patient.

Transcription: As used herein, the term “transcription” refers to the formation or synthesis of an RNA molecule by, e.g., an RNA polymerase using a DNA molecule as a template.

Translation: As used herein, the term “translation” refers to the formation of a polypeptide molecule by a ribosome based upon an RNA template.

Treat and Prevent: As used herein the terms “treat” or “prevent” as well as words stemming therefrom do not necessarily imply 100% or complete treatment or prevention. Rather, there are varying degrees of treatment or prevention of which one of ordinary skill in the art recognizes as having a benefit or therapeutic effect. Also, “prevention” can encompass delaying the onset of the disease, symptom or condition thereof.

Therapeutically effective amount: As used herein, the term “therapeutically effective amount” means an amount effective, at dosages, frequency of administration, and for duration of time necessary to achieve the desired results such that one or more symptoms or biomarkers is improved after treatment.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Untranslated Region: As used herein, the term “untranslated region” or “UTR” refers to regions 5′ or 3′ of the coding sequence which are not translated. As use herein, “UTR” can include, for example, spacers.

Upstream: As used herein, the term “upstream” refers to sequence that is 5′ to a particular sequence.

Vector: As used herein, a “vector” is any molecule or moiety which transports, transduces or otherwise acts as a carrier of a heterologous molecule. Vectors of the present disclosure may be produced recombinantly and may be based on and/or may comprise viral parent or reference sequences. Such parent or reference viral sequences may serve as an original, second, third, or subsequent sequence for engineering vectors. In non-limiting examples, such parent or reference viral sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence.

Vehicle: The term “vehicle” refers to an agent, e.g., a carrier, that may typically be used within a pharmaceutical composition for facilitating administering of the components of the pharmaceutical composition to an individual.

Virus-like Particle: Virus-like particles (VLPs) are molecules that closely resemble viruses but are non-infectious because they contain no viral genetic material. As used herein, the term “virus-like particle” is used interchangeably with “viral like particles.” VLPs can be naturally occurring or synthesized through the individual expression of viral structural proteins, which can then self-assemble into the virus-like structure. As used herein, VLPs are used interchangeably with engineered viral particles.

### DETAILED DESCRIPTION

The present disclosure, provides, among other things, an improved in vivo CAR therapy for treating B cell mediated diseases based on circular ribonucleic acids (circRNA). In some aspects, the present disclosure provides targeted in vivo delivery of genes encoding CARs to select cell types (e.g., immune cells, or, more specifically, T cells) for treating B cell-mediated diseases (e.g., autoimmune disease or cancer). The in vivo CAR therapy disclosed herein provides multiple benefits over current ex vivo CAR therapies, for which manufacturing is complex, laborious, and expensive.

Ex vivo CAR therapies to treat cancer or autoimmune diseases requires patients' or donors' T cells to be isolated from peripheral blood via apheresis, activated, and subsequently transduced by a lentivirus comprising a chimeric antigen receptor (CAR) construct to create an engineered CAR T cell. CAR T cells are further expanded before infusing engineered T cells into a patient's bloodstream to attack target cells. Further, ex vivo therapy requires lymphodepletion prior to administration, and is also associated with serious side-effects including cytokine release syndrome (CRS), which can cause multi-organ failure and even death, and immune effector cell-associated neurotoxicity syndrome (ICANS), which is a neuropsychiatric syndrome that can cause confusion and seizures (Bui, 2024; Mullard, 2024).

The in vivo CAR therapy of the present invention uses the body's own immune system to manufacture the CAR T cells in vivo and bypasses the complex manufacturing steps required for ex vivo therapy. In addition, the in vivo CAR therapy does not require a step of lymphodepletion prior to administration of the therapy to the patient.

The present disclosure, provides, in part, a highly stable, durable, and targeted in vivo CAR therapy encoded by circRNA that is achieved through an unexpected and surprising synergy of a combination of precisely engineered features in the circRNA, delivery vehicle (e.g., lipid nanoparticle), and targeting moiety conjugated to the delivery vehicle. In some embodiments, the targeted in vivo CAR therapy in the present disclosure allows expression of the CAR therapy, and use of circRNA improves the duration and magnitude of CAR expression on T cells, which results in B cell depletion. As described herein, the targeted in vivo CAR therapy reaches the specific cell types and tissues, e.g., T cells, and allows for enhanced in vivo CAR T generation and improves potency for B cell depletion.

The circRNA therapeutics described in this disclosure provide multiple advantages compared to linear RNA. For example, circRNA therapeutics provides enhanced stability. Without wishing to be bound by any mechanism, since circRNA is a closed molecule with no free ends, it confers resistance to exonucleases, which leads to RNA durability. In addition, the absence of open 5′ and 3′ ends leads to potential reduction of an innate immune response by the patient. Moreover, circular RNA allows for efficient translation compared to linear RNA; IRES-mediated translation (cap-independent) leads to high synthesis of the protein encoded by the circular RNA.

The circRNAs and circRNA therapeutics of the present disclosure were precisely engineered to be efficacious and safe to treat diseases such as autoimmune diseases and cancer. In some embodiments, the circRNAs and circRNA therapeutics of the present disclosure: stably and durably express CAR to high levels, in part, due to increased stability of circRNA relative to a linear RNA; have specific Internal Ribosome Entry Site (IRES) elements that drive unexpectedly high expression of the CAR construct, including anti-CD19 CAR construct, in T cells; include variant 5′ and/or 3′ untranslated regions (UTR) with miRNA binding sites in the spacers to decrease off-target liver expression; and are codon-optimized for high expression of the CAR construct, including anti-CD19 CAR construct.

In some aspects, provided herein is a circular ribonucleic acid (circRNA) therapeutic for targeted delivery comprising: (i) a circRNA comprising a ribosome recruiting element, and a coding sequence encoding a chimeric antigen receptor (CAR) comprising an anti-CD19 antibody or antigen-binding fragment thereof, and (ii) a targeting moiety, wherein the circRNA is encapsulated in a delivery vehicle, wherein the targeting moiety is conjugated to the delivery vehicle, and wherein the targeting moiety comprises an anti-CD5 antibody heavy chain variable region comprising CDR1, CDR2, and CDR3 sequences from SEQ ID NO: 89.

In some aspects, provided herein is a circular ribonucleic acid (circRNA) for targeted delivery comprising: (i) a circRNA comprising: a ribosome recruiting element, and a coding sequence encoding a chimeric antigen receptor (CAR) comprising an anti-CD19 antibody or antigen-binding fragment thereof, and (ii) a targeting moiety, wherein the targeting moiety binds to a T cell, wherein the circRNA is in a delivery vehicle, wherein the targeting moiety that binds to a T cell is conjugated to the delivery vehicle, and wherein the targeting moiety is an anti-CD5 antibody or antigen-binding fragment thereof comprising a heavy chain variable region comprising complementarity determining region (CDR) sequences GFTFSMYS (SEQ ID NO: 64), ISTGARDT (SEQ ID NO: 65) and GDLRYGPDGYDY (SEQ ID NO: 66).

Provided herein, among other things, is a circular ribonucleic acid (circRNA) for targeted delivery comprising: (i) a circRNA comprising a coding sequence encoding a chimeric antigen receptor (CAR) comprising an anti-CD19 antibody or antigen-binding fragment thereof, (ii) a targeting moiety, and (iii) a delivery vehicle, wherein the targeting moiety is conjugated to the delivery vehicle (e.g., lipid nanoparticle, engineered viral particle, among others). In some embodiments, the targeting moiety comprises an anti-CD5 antibody or antigen-binding fragment thereof.

The present disclosure provides, among other things, a circRNA, comprising: a coding sequence encoding a chimeric antigen receptor (CAR) comprising an anti-CD19 antibody or antigen-binding fragment thereof, wherein the circRNA comprises an Internal Ribosome Entry Site (IRES) having at least 85% identity to a sequence comprising any one of SEQ ID NO: 1-11, and one or more untranslated region (UTR).

Among other things, provided herein is a circRNA comprising a codon-optimized coding sequence having at least 85% identity to SEQ ID NOS: 125-168 or 266.

Provided herein, in part, is a circRNA comprising a sequence having at least 85% identity to any one of SEQ ID NOs: 175-233, 264 or 269. In some embodiments, the sequence is selected from SEQ ID NOs: 175-233, 264, or 269.

Provided herein, among other things, is a circRNA therapeutic for targeted delivery, comprising: (i) a circRNA comprising a coding sequence encoding a chimeric antigen receptor (CAR) comprising an anti-CD19 antibody or antigen-binding fragment thereof, and (ii) an anti-CD5 T cell targeting moiety; wherein the circRNA is encapsulated in a lipid nanoparticle; and wherein the T cell targeting moiety is conjugated to the lipid nanoparticle.

In some aspects, provided herein is a circular ribonucleic acid (circRNA) therapeutic for targeted delivery comprising: a circular RNA comprising a ribosome recruiting element and a coding sequence encoding a chimeric antigen receptor (CAR) comprising an anti-CD19 antibody or antigen-binding fragment thereof, wherein the circRNA is encapsulated in an LNP, wherein a targeting moiety is conjugated to the LNP, wherein the targeting moiety is an anti-CD5 VHH, the anti-CD5 VHH comprising CDR1, CDR2, and CDR3 sequences from SEQ ID NO: 89, wherein the LNP comprises one or more ionizable lipids at 30 to 49 mol % (e.g, at 30 to 45 mol %), one or more phospholipids at 5 to 40 mol % (e.g., at 15 to 35 mol %), one or more sterol lipids at 15 to 50 mol % (e.g., at 30 to 50 mol %), DMG-PEG 2000 at 0 to 10 mol % (e.g., at 0 to 5 mol %), and DSPE-PEG 2000-maleimide at 0.01 to 1 mol %, and wherein the VHH is conjugated to the LNP.

The present disclosure, provides among other things, a circRNA therapeutic for targeted delivery, comprising: (i) a circRNA comprising (a) a 5′ untranslated region (UTR), (b) an Internal Ribosome Entry Site (IRES) having at least 85% identity to a nucleic acid sequence comprising SEQ ID NO: 1, (c) a coding sequence encoding: a CD8a signal peptide, and a Chimeric Antigen Receptor (CAR) comprising: an anti-CD19 scFv, a CD8a hinge, a CD8a transmembrane domain, a 4-1BB costimulatory domain, a CD3 zeta signaling domain, and (d) a 3′ UTR, and (ii) an anti-CD5 T cell targeting moiety comprising the complementarity determining region (CDR) sequences of GFTFSMYS (SEQ ID NO:64), ISTGARDT (SEQ ID NO: 65) and GDLRYGPDGYDY (SEQ ID NO: 66), wherein the circRNA is encapsulated in a lipid nanoparticle (RNA-LNP), and wherein the anti-CD5 T cell targeting moiety is conjugated to the lipid nanoparticle.

In some aspects, provided herein is a circular ribonucleic acid (circRNA) therapeutic for targeted delivery comprising: a circular RNA comprising a ribosome recruiting element and a coding sequence encoding a chimeric antigen receptor (CAR) comprising an anti-CD19 antibody or antigen-binding fragment thereof, wherein the circRNA is encapsulated in an LNP, wherein a targeting moiety is conjugated to the LNP, wherein the targeting moiety is an anti-CD5 VHH, the anti-CD5 VHH comprising CDR1, CDR2, and CDR3 sequences from SEQ ID NO: 89, wherein the LNP comprises one or more ionizable lipids at 30 to 49 mol %, one or more phospholipids at 5 to 40 mol %, one or more sterol lipids at 15 to 50 mol %, a DMG-PEG 2000 lipid at 0 to 10 mol %, and a DSPE-PEG 2000-maleimide lipid at 0.01 to 1 mol %, and wherein the VHH is conjugated to the LNP.

In some aspects, provided herein is a circular ribonucleic acid (circRNA) therapeutic for targeted delivery comprising: a circular RNA comprising a ribosome recruiting element and a coding sequence encoding a chimeric antigen receptor (CAR) comprising an anti-CD19 antibody or antigen-binding fragment thereof, wherein the circRNA is encapsulated in an LNP, wherein a targeting moiety is conjugated to the LNP, wherein the targeting moiety is an anti-CD5 VHH, the anti-CD5 VHH comprising CDR1, CDR2, and CDR3 sequences from SEQ ID NO: 89, wherein the LNP comprises one or more ionizable lipids at 30 to 45 mol %, one or more phospholipids at 15 to 35 mol %, one or more sterol lipids at 30 to 50 mol %, a DMG-PEG 2000 lipid at 0 to 5 mol %, and a DSPE-PEG 2000-maleimide lipid at 0.01 to 1 mol %, and wherein the VHH is conjugated to the LNP.

In some aspects, provided herein is a circRNA therapeutic, comprising: (i) a circRNA comprising in order: (a) a 5′ exon fragment and a 5′ UTR, (b) an IRES having at least 85% identity to SEQ ID NO: 1, (c) a coding sequence encoding a chimeric antigen receptor (CAR), (d) a 3′ UTR and a 3′ exon fragment, and (ii) an anti-CD5 T cell targeting moiety comprising the complementarity determining region (CDR) sequences CDR1, CDR2, and CDR3, wherein the CDR1, the CDR2, and the CDR3 amino acid sequences are GFTFSMYS (SEQ ID NO: 64), ISTGARDT (SEQ ID NO: 65), and GDLRYGPDGYDY (SEQ ID NO: 66), respectively, wherein the circRNA is encapsulated in a lipid nanoparticle, and wherein an anti-CD5 T cell targeting moiety is conjugated to the lipid nanoparticle.

In some aspects, provided herein is a circRNA therapeutic, comprising: (i) a circRNA comprising in order: (a) a 5′ exon fragment and a 5′ UTR, (b) an IRES having at least 85% identity to SEQ ID NO: 1, (c) a coding sequence encoding a chimeric antigen receptor (CAR) that targets CD19, (d) a 3′ UTR and a 3′ exon fragment, and (ii) an anti-CD5 T cell targeting moiety comprising the complementarity determining region (CDR) sequences CDR1, CDR2, and CDR3, wherein the CDR1, the CDR2, and the CDR3 amino acid sequences are GFTFSMYS (SEQ ID NO: 64), ISTGARDT (SEQ ID NO: 65), and GDLRYGPDGYDY (SEQ ID NO: 66), respectively, wherein the circRNA is encapsulated in a lipid nanoparticle, and wherein an anti-CD5 T cell targeting moiety is conjugated to the lipid nanoparticle.

Provided herein is a circRNA therapeutic for targeted delivery, comprising: (i) a circRNA comprising (a) a 5′ untranslated region (UTR), (b) an Internal Ribosome Entry Site (IRES) having 100% identity to a nucleic acid sequence comprising SEQ ID NO: 1, (c) a coding sequence encoding: a Chimeric Antigen Receptor (CAR) comprising: FMC63 (an anti-CD19 scFv) having a VL sequence of DIQMTQTTSSLSASLGDRVTISCRASQDISKYLNWYQQKPDGTVKLLIYHTSRLHSGVPSRFSG SGSGTDYSLTISNLEQEDIATYFCQQGNTLPYTFGGGTKLEIT (SEQ ID NO: 54), linked by a linker having the amino acid sequence of GGGGSGGGGSGGGGS (SEQ ID NO: 56), and a VH sequence of EVKLQESGPGLVAPSQSLSVTCTVSGVSLPDYGVSWIRQPPRKGLEWLGVIWGS ETTYYNSALKSRLTIIKDNSKSQVFLKMNSLQTDDTAIYYCAKHYYYGGSYAMDYWGQGTSVTV SS (SEQ ID NO: 55), a CD8a hinge, a CD8a transmembrane domain, a 4-1BB costimulatory domain, a CD3 zeta signaling domain, and a CD8 signal peptide; and (d) a 3′ untranslated region (UTR) of SEQ ID NO: 46 (also known as SEQ ID NO: 272) comprising a miR-122 binding site, and (ii) an anti-CD5 T cell targeting moiety comprising the sequence of SEQ ID NO: 89, wherein the circRNA is encapsulated in a lipid nanoparticle, wherein the anti-CD5 T-cell targeting moiety is conjugated to the lipid nanoparticle via a thiol-maleimide conjugation. In some embodiments, the LNP comprises an ionizable lipid, a phospholipid, a sterol lipid, and one or more PEG-lipids.

Also provided by the present disclosure is a precursor RNA comprising, from 5′ to 3′ end: (i) a 3′ Group I or Group II intron splicing fragment, (ii) a 5′ untranslated region (UTR), (ii) an IRES, (iii) a coding sequence encoding a chimeric antigen receptor (CAR) comprising an anti-CD19 antibody or antigen-binding fragment thereof, (iv) a 3′ untranslated region (UTR), and (v) a 5′ Group I or Group II intron splicing fragment, wherein the 3′ and 5′ Group I intron splicing fragments are derived from a Group I or Group II intron.

Provided herein, among other things, is a precursor RNA comprising, from 5′ to 3′ end: (i) a 3′ Group I intron splicing fragment, (ii) a 5′ untranslated region (UTR), (iii) an IRES, (iv) a coding sequence encoding a chimeric antigen receptor (CAR) comprising an anti-CD19 antibody or antigen-binding fragment thereof, (v) a 3′ untranslated region (UTR), and (vi) a 5′ Group I intron splicing fragment, wherein the 3′ and 5′ Group I intron splicing fragments are derived from a Twort-ORF142 Group I intron. As used herein, the terms “intron splicing fragment” and “intron fragment” are used interchangeably. The Twort-ORF142 intron sequence includes nucleotides 1055-1336 of the open reading frame Twort-ORF142 (unknown gene) in the genome of Staphylococcus phage Twort (GenBank: AF132670.1) (Landthaler and Shub, PNAS, 1999, 96 (12), 7005-7010):

(SEQ ID NO: 273)
AACTACTGAAAGCATAAATAATTGTGCCTTTATACAGTAATGTATATCG
AAAAATCCTCTAATTCAGGGAACACCTAAACAAACTAAGATGTAGGCAA
TCCTGAGCTAAGCTCTTAGTAATAAGAGAAAGTGCAACGACTATTCCGA
TAGGAAGTAGGGTCAAGTGACTCGAAATGGGGATTACCCTTCTAGGGTA
GTGATATAGTCTGAACATATATGGAAACATATAGAAGGATAGGAGTAAC
GAACCTATTCGTAACATAATTGAACTTTTAGTTATTT.

In some embodiments, the nucleic acid molecule for making a circular RNA comprises the upstream intron sequence comprising a sequence corresponding to positions 119 to 282 of SEQ ID NO: 273, and the downstream intron sequence comprising a sequence corresponding to positions 1 to 118 of SEQ ID NO: 273. In one non-limiting example, the upstream intron sequence corresponds to positions 119 to 282 of SEQ ID NO: 273, and the downstream intron sequence corresponds to positions 1 to 118 of SEQ ID NO: 273. In some embodiments, the upstream intron fragment comprises a sequence corresponding to positions 147 to 282 of SEQ ID NO: 273, and the downstream intron fragment comprises a sequence corresponding to positions 1 to 146 of SEQ ID NO: 273. In one non-limiting example, the upstream intron sequence corresponds to positions 147 to 282 of SEQ ID NO: 273, and the downstream intron sequence corresponds to positions 1 to 146 of SEQ ID NO: 273. Incorporated herein by reference in entirety is the disclosure in co-pending patent application U.S. 63/660,303 by Applicants (entitled “Compositions and Methods for RNA Circularization”).

### Circular RNA for Targeted Delivery

The present disclosure provides, in part, a circular ribonucleic acid (circRNA) for targeted delivery comprising: (i) a circRNA comprising a coding sequence encoding a chimeric antigen receptor (CAR) comprising an anti-CD19 antibody or antigen-binding fragment thereof, (ii) a targeting moiety, and (iii) a delivery vehicle, wherein the targeting moiety is conjugated to the delivery vehicle (e.g., lipid nanoparticle, viral particle, among others).

In some aspects, provided herein is a circular ribonucleic acid (circRNA) therapeutic for targeted delivery comprising: (i) a circRNA comprising a ribosome recruiting element, and a coding sequence encoding a chimeric antigen receptor (CAR) comprising an anti-CD19 antibody or antigen-binding fragment thereof, and (ii) a targeting moiety, wherein the circRNA is encapsulated in a delivery vehicle, wherein the targeting moiety is conjugated to the delivery vehicle, and wherein the targeting moiety comprises an anti-CD5 antibody heavy chain variable region comprising CDR1, CDR2, and CDR3 sequences from SEQ ID NO: 89.

In some embodiments, the CDR1 sequence comprises SEQ ID NO: 64, the CDR2 sequence comprises SEQ ID NO: 65, and the CDR3 sequence comprises SEQ ID NO: 66.

### Circular RNA

In some embodiments, the present invention provides a nucleic acid molecule for making a circRNA molecule that encodes an anti-CD19 CAR polypeptide. In some embodiments, the nucleic acid molecule is a DNA construct (e.g., a vector) that is transcribed into linear RNA, and the linear RNA circularizes into a circular RNA. In some embodiments, the nucleic acid molecule is a linear precursor RNA polynucleotide that circularizes into a circular RNA. The nucleic acid molecule described herein comprises different elements essential for the circular RNA synthesis and function. In some embodiments, the present invention provides a circular RNA encoding an anti-CD19 CAR polypeptide that is made from a nucleic acid molecule or by a method described herein.

In some embodiments, the nucleic acid molecule for making a circular RNA comprises self-splicing intron sequences and a coding sequence. A rational design of a synthetic circular RNA polynucleotide cassette includes at least two self-splicing intron sequences flanking the sequence to be circularized. The upstream and downstream intron sequences are self-spliced to generate a circular RNA comprising the coding sequence. In some embodiments, a 5′ untranslated region and/or 3′ untranslated region (UTR)) sequences are included between the intron sequences and the coding sequence (i.e., the sequence to be circularized). In some embodiments, one or more internal ribosome entry site (IRES) is added and operably linked to the coding sequence.

In some embodiments, the circRNA encodes an amino acid sequence having at least 75% identity to any one of SEQ ID NOs: 169-174. In some embodiments, the circRNA encodes an amino acid sequence having at least 80% identity to any one of SEQ ID NOs: 169-174. In some embodiments, the circRNA encodes an amino acid sequence having at least 85% identity to any one of SEQ ID NOs: 169-174. In some embodiments, the circRNA encodes an amino acid sequence having at least 95% identity to any one of SEQ ID NOs: 169-174. In some embodiments, the circRNA encodes an amino acid sequence having at least 99% identity to any one of SEQ ID NOs: 169-174. In some embodiments, the circRNA encodes an amino acid sequence of any one of SEQ ID NOs: 169-174.

In some embodiments, provided herein is a circRNA comprising a sequence having at least 80% identity to any one of SEQ ID NOs: 175-233, 264 or 269. In some embodiments, provided herein is a circRNA comprising a sequence having at least 85% identity to any one of SEQ ID NOs: 175-233, 264 or 269. In some embodiments, provided herein is a circRNA comprising a sequence having at least 90% identity to any one of SEQ ID NOs: 175-233, 264 or 269. In some embodiments, provided herein is a circRNA comprising a sequence having at least 95% identity to any one of SEQ ID NOs: 175-233, 264 or 269. In some embodiments, the circRNA comprises a sequence having 100% identity to any one of SEQ ID NOs: 175-233, 264 or 269.

In some embodiments, the untranslated region (UTR) comprises a 5′ UTR, a 3′ UTR or both a 5′ UTR and a 3′ UTR. In some embodiments, the UTR comprises a 5′ UTR. In some embodiments, the UTR comprises a 3′ UTR.

In some embodiments, the UTR comprises a CAA40 comprising SEQ ID NO: 42 or a pAC18 comprises SEQ ID NO: 43 (also known as SEQ ID NO: 265). In some embodiments, the UTR comprises any one of SEQ ID NO: 43-45, 46 or 47. In some embodiments, the UTR comprises SEQ ID NO: 46 (also known as SEQ ID NO: 272) or SEQ ID NO: 47. In some embodiments, the UTR comprises one or more microRNA binding sites. In some embodiments, the one or more microRNA binding sites is selected from any one of SEQ ID NOs: 27-40.

In some embodiments, the circRNA further comprises one or more microRNA binding sites in the 5′ UTR, the 3′ UTR, or both. In some embodiments, the microRNA binding site is a miR-122 site. In some embodiments, the miR-122 binding site is in the 3′ UTR. In some embodiments, the miR-122 binding site is in the 5′ UTR and 3′ UTR.

In some aspects, provided herein is a circular ribonucleic acid (circRNA) comprising: (a) a coding sequence encoding a chimeric antigen receptor (CAR) comprising an anti-CD19 antibody or antigen-binding fragment thereof, (b) an Internal Ribosome Entry Site (IRES) comprising a sequence at least 85% identical to any one of SEQ ID NO: 1-11, (c) 3′ and 5′ untranslated region(s) (UTR), (d) 3′ and 5′ exon fragments, and (e) a single microRNA binding site.

In some aspects, provided herein isa circular ribonucleic acid (circRNA) comprising in order: (a) a 5′ exon fragment and 5′ untranslated region (UTR), (b) an Internal Ribosome Entry Site (IRES) comprising a sequence at least 85% identical to any one of SEQ ID NO: 1-11, (c) a coding sequence encoding a chimeric antigen receptor (CAR) comprising an anti-CD19 antibody or antigen-binding fragment thereof, (d) a 3′ UTR and a 3′ exon fragment, and (e) a single microRNA binding site within the 5′ UTR or the 3′ UTR.

In some aspects, provided herein isa circular ribonucleic acid (circRNA) comprising in order: (a) a 5′ exon fragment and 5′ untranslated region (UTR), (b) an Internal Ribosome Entry Site (IRES) comprising a sequence at least 85% identical to any one of SEQ ID NO: 1-11, (c) a coding sequence encoding a chimeric antigen receptor (CAR) comprising an anti-CD19 antibody or antigen-binding fragment thereof, (d) a 3′ UTR and a 3′ exon fragment, and (e) a single microRNA binding site within the 5′ UTR or the 3′ UTR.

### Ribosome Recruiting Element

In some embodiments, the circRNA comprises a ribosome recruiting element. As used here, a “ribosome recruiting element” includes naturally occurring or engineered RNA sequences that directly or indirectly recruit the ribosome to initiate translation of RNA into protein. In some embodiments, a ribosome recruiting element is an internal ribosome entry site (IRES). In other embodiments, a ribosome recruiting element is a branched cap structure (e.g., qRNA) added to circular RNA that initiates translation by the ribosome. In some embodiments, a ribosome recruiting element is an IRES transacting factor (ITAF) recruiting element. In some embodiments, IRES and ITAFs recruit the ribosome. In some embodiments, the ITAF is specific to an IRES or group of related IRES elements. In some embodiments, the ITAF is general (for example, polypyrimidine tract-binding protein, PTB).

### Internal Ribosome Entry Site

In some embodiments, the ribosome recruiting element is an Internal Ribosome Entry Site (IRES). In some embodiments, the circRNA comprises an Internal Ribosome Entry site (IRES). Because circRNA is generated by spliceosome-mediated head-to-tail joining of pre-mRNAs, it does not contain the 5′ cap that is commonly known to be required for cap-dependent translation. Thus, circRNA translation utilizes alternate mechanisms to initiate cap-independent translation, such as the use of an internal ribosome entry site (IRES) sequence that is recognized by ribosomes. Introduction of an IRES on synthetically generated circRNAs is sufficient to initiate translation of encoded circRNA proteins, thereby suggesting that endogenous circRNAs harboring IRES sequence may have translation potential as they are exported to the cytoplasm. As used herein, the term “internal ribosome entry site” or “IRES” refers to an RNA sequence or structural element ranging in size from 10 nucleotides to 1,000 nucleotides or more which is capable of initiating translation of a polypeptide in the absence of a normal RNA cap structure. Inclusion of an IRES element in circRNA engages a eukaryotic ribosome for translation, i.e., the IRES element initiates cap-independent translation and protein synthesis.

Notably, IRES function is cell-type dependent, i.e., the efficiency of IRES-mediated translation can vary significantly depending on the specific cell type due to the presence of different cellular transactivating factors, under stress conditions or developmental stage of the cell. In the present disclosure, IRES elements for optimal T cell function in activated and resting T cells and with breadth of function in different donors are carefully selected through experimentation.

In some embodiments, the IRES is a synthetic IRES. In some embodiments, the IRES is a viral IRES. In some embodiments, the IRES is a naturally-occurring viral IRES. In some embodiments, the IRES is a recombinant variant of a naturally-occurring viral IRES. In some embodiments, the IRES is a fragment of the full-length sequence of a naturally-occurring viral IRES. In some embodiments, the IRES includes but is not limited to an IRES derived from Apodemus agrarius picornavirus, Canine kobuvirus, Picornavirales/Parabovirus, Caprine kobuvirus, Mouse kobuvirus, Bejaponia (bovine) kobuvirus, Human Enterovirus C99, Bovine rhinitis A virus, Kobuvirus aichi, Human coxsackievirus A21, Falcon picornavirus, Aichivirus B, Rattus tanezumi hunnivirus, Human coxsackievirus A5, Human poliovirus 2, Enterovirus B69, Saffold virus 3, Salivirus FHB, or Human Echovirus E3, or variants or fragments derived from these viruses.

In some embodiments, the ribosome recruiting element is an Internal Ribosome Entry Site (IRES), wherein the IRES comprises a sequence selected from Table 1.

In some embodiments, the IRES comprises a sequence selected from Table 1. In some embodiments, the IRES comprises any one of the sequences in Table 1. In some embodiments, the IRES comprises any one of the sequences selected from SEQ ID NO: 1-11 of Table 1. In some embodiments, the IRES comprises a sequence selected from SEQ ID NO: 1-11 of Table 1. In some embodiments, the IRES comprises a sequence having at least 90% identity to any one of SEQ ID NO: 1-11. In some embodiments, the IRES comprises a sequence having at least 95% identity to any one of SEQ ID NO: 1-11. In some embodiments, the IRES comprises a sequence of any one of SEQ ID NO: 1-11.

In some embodiments, the IRES comprises a sequence having at least 90% identity to any one of SEQ ID NO: 1-6. In some embodiments, the IRES comprises a sequence having at least 95% identity to any one of SEQ ID NO: 1-6. In some embodiments, the IRES comprises a sequence of any one of SEQ ID NO: 1-6. In some embodiments, IRES comprises a sequence having 95% identity to SEQ ID NO: 1. In some embodiments, the IRES comprises the sequence of SEQ ID NO: 1. In some embodiments, the IRES comprises SEQ ID NO: 1.

In some embodiments, the IRES comprises a sequence having at least 90% identity to SEQ ID NO: 354. In some embodiments, the IRES comprises a sequence having at least 95% identity to SEQ ID NO: 354. In some embodiments, the IRES comprises a sequence having 100% identity to SEQ ID NO: 354. In some embodiments, the IRES comprises a sequence of SEQ ID NO: 354.

In some embodiments, the IRES is associated with higher CAR expression in T cells in comparison to a reference Coxsackievirus B3 IRES (SEQ ID NO: 12). In some embodiments, the IRES comprising any one of SEQ ID NO: 1-11 is associated with higher CAR expression in T cells in comparison to a reference Coxsackievirus B3 IRES (SEQ ID NO: 12). In some embodiments, the IRES comprising SEQ ID NO: 1 is associated with higher CAR expression in T cells in comparison to a reference Coxsackievirus B3 IRES (SEQ ID NO: 12).

TABLE 1
Exemplary Internal Ribosome Entry Site (IRES) RNA Sequences

IRES263
TTACTCAGCGTAACTACTCCGGGTTACGTGATGAAGAAGAGGCTACGGAGATTCTCGGGCTACGGCCCTGGAGCCA
CTCCGGCTCCTAAAGATTTAGAAGTTTGAGCACACCCGCCCACTAGGGCCCCCCATCCAGGGGGGCAACGGGCAAG
CACTTCTGTTTCCCCGGTATGATCTGATAGGCTGTAACCACGGCTGAAACAGAGATTATCGTTATCCGCTTCACTA
CTTCGAGAAGCCTAGTAATGATGGGTGAAATTGAATCCGTTGATCCGGTGTCTCCCCCACACCAGAAACTCATGAT
GAGGGTTGCCATCCCGGCTACGGCGACGTAGCGGGCATCCCTGCGCTGGCATGAGGCCTCTTAGGAGGACGGATGA
TATGGATCTTGTCGTGAAGAGCCTATTGAGCTAGTGTCGACTCCTCCGCCCCCGTGAATGCGGCTAATCCTAACCC
CGGAGCAGGTGGGTCCAATCCAGGGCCTGGCCTGTCGTAATGCGTAAGTCTGGGACGGAACCGACTACTTTCGGGA
AGGCGTGTTTCCATTTGTTCATTATTTGTGTGTTTATGGTGACAACTCTGGGTAAACGTTCTATTGCGTTTATTGA
GAGATTCCCAACAATTGAACAAACGAGAACTACCTGTTTTATTAAATTTACACAGAGAAGAATTACA (SEQ ID
NO: 1)
IRES318
TTTCCCCTGTTCGTAACTAAGTGTGTGCCCAATCTCCTCACTCCTGCTGGCTTCACCGACCGGCAGTGTCCAAAAT
GCTAGGTGAATCCCCTCCCTTTCCTCTGGGCTTCTGCCCAGCTTCCTCCCCCCAGCCTGACGTGACACAGGCTGTG
CAAAGACCCCGCGAAAGCTGCCAAAAGTGGCAATTGTGGGTCCCCCCTTTGTAAAGGCGTCGAGTCTTTCTCCCTC
AAGGCTAGACCCGTCAGTGAATTCTGTCGGGCAACTAGTGACGCCACTGCACGCCTCTGACCTCGGCCGCGGAGTG
CTGCCCCCCAAGTCGTGCCCCTGACCACAAGTTGTGCTGTCTGGCAAACATTGTCTGTGAGAATGTTCCGCTGTGG
CTGCCAAGCCTGGCAACAGGCTGCCCCAGTGTGCGTAGTTCTCATCCAGACTTCGGTCTGGCAACTTGCTGTTAAG
ACACGGCGTAAGGGGCGTGTGCCAACGCCCTGGAACGAGTGTCCACTCTAATACCCCGAGGAATGCTACGCAGGTA
CCCCTGGTTCGCCAGGGATCTGAGCGTAGGCTAATTGTCTAAGGGTATTTTCATTTCCCATTCTTTCTTTCTTGTT
CATA (SEQ ID NO: 2)
IRES303
TGAACCGTTACGCACCACTCAGTTGGTGTTTGGTGGCACCAATGATGGAACAAAAGGCTACACCACTTGGGCTACG
GCCCGCGCCACCTTGTGGCGCAAAGACATTAGAAGAATAGCATACCGCCCACTAGGGCCCTGCAGCCAGCAGGGTA
ACGGGCAAGCACTTCTGTCTCCCCGGTAGAACGGTATAGGCTGTACCCACGGCCGAAAACTGAACTATCGTTACCC
GACTCCGTACTTCGCAAAGCTTAGTAGGAAACTGGAAAGTTCGAGTTATTGACCCGGAGTGTTCCCCCCACTCCAG
AAACGCGTGATGAGGGTTGCCACCCCGACCATGGCGACATGGTGGGCATCCCTGCGCTGGCACGCGGCCTCTAAGA
GGATAACTCGCTCCTACTGGTAACCGAAGAGCCCCGTGAGCTACGGTTTATTCCTCCGCCTCCCTGAATGCGGCTA
ATCCTAACCCATGAGCAGTTGCCATAGATCCATATGGTGGACTGTCGTAACGCGTAAGTIGTGGGCGGAACCGACT
ACTTTGGGATGGCGTGTTTCCTTGTTTTCTCCATTTGTTGTTGTATGGTGACAAGITATAGATCTCGATCTATAGC
GTTTCTTGAGAGATTTCCAAACATTTATTCAAGTCGTACAATTCTTGTGTTTAAGCAGTACAGTGTAAGG (SEQ
ID NO: 3)
IRES124
TTTAAGTGTTGTGCCCAATCTCTTGACTCCTGCTGGAACCACCGACCAGTAGTGTCCAAAATGCCAGGTGGAAAAT
CCTCCCTTCCCCTCTGGGCTTCATGCCCGGCATCCTCCCCCCAGCCTGACGTGCCACAGGCTGTGCAAAGACCCCG
CGAAAGCTGCCAAAAGTGGCAATTGTGGGTCCCCCCTTTGTCAAGGCGTCGAGTCTTTCTCCCTTAAGGCTAGTCC
TGTCAGTGAACTCTGTCGGGCAACTAGTGACGCCACTGCATGCCTCCGACCTCGGCCGCGGAGTGCTGCCCCCCAA
GTCATGCCCCTGACCACAAGTIGTGCTGTCTGGCAAACATTGTCTGTGAGAATGTTCCGCTGTGGCTGCCAAGCCT
GGTAACAGGCTGCCCCAGTGTGCGTAATTCTCATCCAGACTTCGGTCTGGCAACTTGCTGTTAAGACATGGCGTAA
GGGGCGTGTGCCAACGCCCTGGAACGAGTGTCCACTCTAATACCCCGAGGAATGCTACGCAGGTACCCCTGGCTCG
CCAGGGATCTGAGCGTAGGCTAATTGTCTAAGGGTATTTTCATTTCCCACCCTCTTCTTCTTGTTCATA (SEQ
ID NO: 4)
IRES302
TTTGCTCAGCGTAACTTCTCCGGGTTACGTGGAGACCAAAAGGCTACGGAGACTCGGGCTACGGCCCTGGAGCACC
TAGGTGCTCCTAAAGACGTTAGAAGTTGTACAAACTCGCCCAATAGGGCCCCCCAACCAGGGGGGTAGCGGGCAAG
CACTTCTGTTTCCCCGGTATGATCTCATAGGCTGTACCCACGGCTGAAAGAGAGATTATCGTTACCCGCCTCACTA
CTTCGAGAAGCCCAGTAATGGTTCATGAAGTTGATCTCGTTGACCCGGTGTTTCCCCCACACCAGAAACCTGTGAT
GGGGGTGGTCATCCCGGTCATGGCGACATGACGGACCTCCCCGCGCCGGCACAGGGCCTCTTCGGAGGACGAGTGA
CATGGATTCAACCGTGAAGAGCCTATTGAGCTAGTGTTGATTCCTCCGCCCCCGTGAATGCGGCTAATCCCAACTC
CGGAGCAGGCGGGCCCAAACCAGGGTCTGGCCTGTCGTAACGCGAAAGTCTGGAGCGGAACCGACTACTTTCGGGA
AGGCGTGTTTCCTTTTGTTCCTTTTATCAAGTTTTATGGTGACAACTCCTGGTAGACGTTTTATTGCGTTTATTGA
GAGATTTCCAACAATTGAACAGACTAGAACCACTTGTTTTATCAAACCCTCACAGAATAAGATAACA (SEQ ID
NO: 5)
IRES290
CCCCCTTTTCCCTAACCCCAACCGCCGTATCTGGTGGCGGCAAGACACACGGGTCTTTCCCTCTAAAGCACAAATG
TGTGTGTGTCCCAGGTCCTCCTGCGCTCGGTGCGGGAGTGCTCCCACCCAACTGTTGTAAGCCTGTCCAACGTGTC
GTCCTGGCAAGACTATGACGTCGCATGTTCCGCTGCGGATGCCGACCGGGTAACCGGTTCCCCAGTGTGTGTAGTG
CGATCTTCCAGGTTCCCCTGGTTGGCGTTGTCCAGAAACTGCTTCAGGTAAGTGGGGTGTGCCCAATCCCTGCAAA
GGTTGATTCTCTCACCACCTTAGGAATGCTCCGGAGGTACCCCAGCAACAGCTGGGATCTGACCGGAGGCTAATTG
TCTACGGGTGGTGTTTCCTTTTTCTTTTTCCACAACTTCATTGCTGACAACTCACTGACTACCCATTTGCTCTCTT
GTGCCTTTCTGCTCTGGTTCAAGCTCCTTGATTGTTTTTGATTGCTTTCTATTGCTTTCTTCCACTCAACCCTTGC
TCAGTTCAAAGTC (SEQ ID NO: 6)
IRES361
TTCAAGTGTGTGTGCTCGTAATCTTGACTCCTGCCGGAATGCCGCCCGGTTCAGTGAACAAACAGCTAGGCAAGTC
CCTCCCTTCCCCTGTGGTCGGTTCTCACCGGCCACCATCCCTCCCCCAGCCTGACGTGTTACAGGCTGTGCAAAGC
CCCCGCGAAAGCTGCTCACGTGGCAATTGTGGGTCCCCCCTTTGTCAAGACACCGAGTCTTTCTCCCTTAAGGCTA
GCCCGGTCCCACGAACGTGGAACTGGCAACTAGTGGTGTCACTACACGCCTCCGACCTCGGACGCGGAGTGCTGTT
CCCCAAGCTGTAACCCTGACCCAAGACTGTGCTGCCTGGCAAGCACCGTCTGGGAAGATGTTCCGCTGTGGCTGCC
AAACCTGGTAACAGGTGCCCCAGTGTGTGTAGTCTTCCTCCAGTCTCCGGACTGGCAGTCTTGTGTAAAGATGCAG
TGTAAGGTTCAAGTGCCAAATCCCTGGAAGGAGTGACCCTCTACTGCCCTAGGAATGCTGTGCAGGTACCCCCAAC
TTCGGTTGGGGATCTGAGCACAGGCTAATTGTCTACGGGTAGTTTCATTTCCCATCCTCTCTTTTTTGGCATC
(SEQ ID NO: 7)
IRES289
GCCCGTCCCCCTCACCCTCTTTTCCGGTGGCCACGCCCGGGCCACCGATACTTCCCTTCACTCCCTCGGGACTGTT
GGGGAGGAACACAACAGGGCTCCCCTGTATTTCCTCTTCCCATTCCCCCTTTCCTAACCCCAACCGCCGTATCTGG
TGGCGGTAAGACACACGGGTCTTTCCCTCTAAAGCACAATTGTGTGTGTGTCCCAGGTCCTCCTGCGTTCGGTGCG
GGAGTGCTCCCACCCAACTGTTGTAAGCCTGTCCAACGTGTCGTCCTGGCAAGACTATGACGTCGCATGTTCCGCT
GTGGATGCCGACCGGGTAACCGGTTCCCCAGTGTGTGTAGTGCGATCTTCCAGGTTCTCCTGGTTGGCGTTGTCCA
GAAACTGCTTCGGGTAAGTGGGGTGTGCCCAATCCCTACAAGGGTTGATTCTTTCACCACCTTAGGAATGCTCCGG
AGGTACCCCAGCAACAGCTGGGATCTGACCGGAGGCTAATTGTCTACGGGTGGTGTTTCCATTTTCTTTTTCACAC
AACTTCATTGCTGACAACTCACTGACTAATCACTTGCTCTCTTGTGCCTTTCTGCTCTGGTTCAAGTTCCTTGATT
GTTTGTTTGATTGCTTTTCACTGCTTTCTTCCCACAATCCTTGCTCAGTTCAAAGTC (SEQ ID NO: 8)
IRES644
TTCCCCCTTTTCCCAACCCCAACCGCCGTATCTGGTGGCGGCAAGACACACGGGTCTTTCCCTCTAAAGCACAATT
GTGTGTGTGTCCCAGGTCCTCCTGCGTACGGTGCGGGAGTGCTCCCACCCAACTGTTGTAAGCCTGTCCAACGCGT
CGTCCTGGCAAGACTATGACGTCGCATGTTCCGCTGCGGATGCCGACCGGGTAACCGGTTCCCCAGTGTGTGTAGT
GCGATCTTCCAGGTCCTCCTGGTTGGCGTTGTCCAGAAACTGCTTCAGGTAAGTGGGGTGTGCCCAATCCCTACAA
AGGTTGATTCTTTCACCACCTTAGGAATGCTCCGGAGGTACCCCAGCAACAGCTGGGATCTGACCGGAGGCTAATT
GTCTACGGGTGGTGTTTCCTTTTTCTTTTCACACAACTCTACTGCTGACAACTCACTGACTATCCACTTGCTCTCT
TGTGCCTTTCTGCTCTGGTTCAAGTTCCTTGATTGTTTTTGACTGCTTTTCACTGCTTTTCTTCTCACAATCCTTG
CTCAGTTCAAAGTC (SEQ ID NO: 9)
IRES596
GTAACTTCAAGTGTGTGTGCTCGTAATCTTGACTCCTGCCGGAATGCCGCCCGGTTCAGTGAACAAACAGCTAGGC
AAGTCCCTCCCTTCCCCTGTGGTCGGTTCTCACCGGCCACCATCCCTCCCCCAGCCTGACGTGTTACAGGCTGTGC
AAAGCCCCCGCGAAAGCTGCTCACGTGGCAATTGTGGGTCCCCCCTTTGTCAAGACACCGAGTCTTTCTCCCTTAA
GGCTAGCCCGGTCCCACGAACGTGGAACTGGCAACTAGTGGTGTCACTACACGCCTCCGACCTCGGACGCGGAGTG
CTGTTCCCCAAGCTGTAACCCTGACCCAAGACTGTGCTGCCTGGCAAGCACCGTCTGGGAAGATGTTCCGCTGTGG
CTGCCAAACCTGGTAACAGGTGCCCCAGTGTGTGTAGTCTTCCTCCAGTCTCCGGACTGGCAGTCTTGTGTAAAGA
TGCAGTGTAAGGTTCAAGTGCCAAATCCCTGGAAGGAGTGACCCTCTACTGCCCTAGGAATGCTGTGCAGGTACCC
CCAACTTCGGTTGGGGATCTGAGCACAGGCTAATTGTCTACGGGTAGTTTCATTTCCCATCCTCTCTTTTTTGGCA
TCATGGCGAAC (SEQ ID NO: 10)
IRES665
CCCCCTCACCCTCTTTTCCGGCGGCGCATGTTCGCGTCGTCGTAAGTCTGGATTCCCAAGGCCCACTCGGTTCAAC
TTCGGTTTCCGGACAAATACAAAGAACCTCAGTCCTCTTGGTACTTTCTCGCCTGAGTCTACAAAGCGAGAAACCT
GCCCCTCTAACGCCAGACGAGCGGCATAAAACTCGAACTTCTGGCACGTTCCACCACCCCTTCTCCTATCCCAACC
CCCATTGCGCTCTCAAGGTCGCGCTTTTCCGAGACTAGCTCGGATTCAAAAAGTTCCTGGCACCCTTTACCCCTTC
AGGCCCTTAAGGTAGGAACTGACCTTGTGCTGTGATCTCGGTGCGGAAGTGCTACTGCGTAGTGATTGTAAGATCC
TTTTGTGGTTCTGCCCTGGCAAGGCTACAGAGTGCTGTGATCCGCTGCGGATGCCATCCTGGTAACAGGACCCCCA
GTGTGCGCAACAGTATGTTCACGGTCTTCCGTGTTCACCACATTCGGAACACTGCTTTCGTGAAACAGTGTGTGTC
CAATCCCTGTGATCAGTATCAACCACACCACCTAGGAATGCTAGGAAGGTACCCCGGTTCGCCGGGATCTGATCCT
AGGCTAATTGTCTACGGTGGTGCTCCTTTTTATTTTCCACTTCAACTCACTGGTTACAACTGCTTGATTTCTGTGT
TTGCTGCTTTTCTCTGCTCTCACTGCCATTCTCAAGTGTTCACACTGTCCAAGCTCCTTTGGTTGTTCGCTTCCAC
TTGCCACTGTCAACTCTTGTC (SEQ ID NO: 11)
Coxsackievirus B3 (CVB3) Reference IRES
TTAAAACAGCCTGTGGGTTGATCCCACCCACAGGCCCATTGGGCGCTAGCACTCTGGTATCACGGTACCTTTGTGC
GCCTGTTTTATACCCCCTCCCCCAACTGTAACTTAGAAGTAACACACACCGATCAACAGTCAGCGTGGCACACCAG
CCACGTTTTGATCAAGCACTTCTGTTACCCCGGACTGAGTATCAATAGACTGCTCACGCGGTTGAAGGAGAAAGCG
TTCGTTATCCGGCCAACTACTTCGAAAAACCTAGTAACACCGTGGAAGTTGCAGAGTGTTTCGCTCAGCACTACCC
CAGTGTAGATCAGGTCGATGAGTCACCGCATTCCCCACGGGCGACCGTGGCGGTGGCTGCGTTGGCGGCCTGCCCA
TGGGGAAACCCATGGGACGCTCTAATACAGACATGGTGCGAAGAGTCTATTGAGCTAGTTGGTAGTCCTCCGGCCC
CTGAATGCGGCTAATCCTAACTGCGGAGCACACACCCTCAAGCCAGAGGGCAGTGTGTCGTAACGGGCAACTCTGC
AGCGGAACCGACTACTTTGGGTGTCCGTGTTTCATTTTATTCCTATACTGGCTGCTTATGGTGACAATTGAGAGAT
CGTTACCATATAGCTATTGGATTGGCCATCCGGTGACTAATAGAGCTATTATATATCCCTTTGTTGGGTTTATACC
ACTTAGCTTGAAAGAGGTTAAAACATTACAATTCATTGTTAAGTTGAATACAGCAAA (SEQ ID NO: 12)
IRES004 Exemplary Control scrambled IRES
TGTAGGTAGAAGGTATAAACGACCAGCCACACACTTCCTACCCAAAGCGCCAATAACAGGAGCCCGGATTGGTCTG
GCACGCAAAGGAGTAGACATCCCCCAATGTTTTCCGAACCAAGCGGCAGCAGCGTCGGTTAGGATCCTCATAATTC
TGATCGAAGGACAGTACACGCGCTACTCAGGGTCTTCTATGATGATTACGATTTACTTACGACGGTGTGATGTAAC
GCCCTCGGCGACTTACTTGGACCTGTTCCCGAATTGAGAATGTGTAGGCCAGCGACCAATCGCCACCACGAGGCCT
AGACTGATCAACTTCAACTGTGAGCGACTCGCATCTGAGGAGTTGACCTAGCTAACACTTCCCATCCACTCTAATT
TGGCTAACAAGGCTGCCACGTGATGATCCTTCTTCTAGGGTGTCTACCTGAGTAAGGGTACCCTTCTCGGAATAAC
GATAAGCGAAGGAGGACATGAACCTAGGCTGGATGTGATTAGCTCCGTAAGCTGCCAACCTGGAGCGCGTCAGATT
CGGCCAACGGCAGATTGGGCTGATCGGGAGTGAGGAATCCCTGCTTACGCTCACTGGGTGATTCCACGTAAAATGT
CCAGTTATTATCTGTGTCCTCTTAACACATTCTTCTGTTTAAGCTTTTCGCCCGTTCTATAAACCTTGTCTGACTA
AGTCCTTAGTCCGCTGCCATTACCGATTGGGTTGCGTAGGCAGCTTCTATTGAAACG (SEQ ID NO: 263)
IRES642
GGCCTCGGCCCCCTCACCCTCTTTTCCGGTGGCCACGCCCGGGCCACCGATACTTCCCTTCACTCCTTCGGGACTG
TTGGGGAGGAACACAACAGGGCTCCCCTGTTTTCCCATTCCTTCCCCCTTTTCCCAACCCCAACCGCCGTATCTGG
TGGCGGCAAGACACACGGGTCTTTCCCTCTAAAGCACAATTGTGTGTGTGTCCCAGGTCCTCCTGCGTACGGTGCG
GGAGTGCTCCCACCCAACTGTTGTAAGCCTGTCCAACGCGTCGTCCTGGCAAGACTATGACGTCGCATGTTCCGCT
GCGGATGCCGACCGGGTAACCGGTTCCCCAGTGTGTGTAGTGCGATCTTCCAGGTCCTCCTGGTTGGCGTTGTCCA
GAAACTGCTTCAGGTAAGTGGGGTGTGCCCAATCCCTACAAAGGTTGATTCTTTCACCACCTTAGGAATGCTCCGG
AGGTACCCCAGCAACAGCTGGGATCTGACCGGAGGCTAATTGTCTACGGGTGGTGTTTCCTTTTTCTTTTCACACA
ACTCTACTGCTGACAACTCACTGACTATCCACTTGCTCTCTTGTGCCTTTCTGCTCTGGTTCAAGTTCCTTGATTG
TTTTTGACTGCTTTTCACTGCTTTTCTTCTCACAATCCTTGCTCAGTTCAAAGTC (SEQ ID NO: 318)
IRES653
CCCCCTTTTCCCAACCCCAACCGCCGTATCTGGTGGCGGCAAGACACACGGGTCTTTCCCTCTAAAGCACAATTGT
GTGTGTGTCCCAGGTCCTCCTGCGTACGGTGCGGGAGTGCTCCCACCCAACTGTTGTAAGCCTGTCCAACGCGTCG
TCCTGGCAAGACTATGACGTCGCATGTTCCGCTGCGGATGCCGACCGGGTAACCGGTTCCCCAGTGTGTGTAGTGC
GATCTTCCAGGTCCTCCTGGTTGGCGTTGTCCAGAAACTGCTTCAGGTAAGTGGGGTGTGCCCAATCCCTACAAAG
GTTGATTCTTTCACCACCTTAGGAATGCTCCGGAGGTACCCCAGCAACAGCTGGGATCTGACCGGAGGCTAATTGT
CTACGGGTGGTGTTTCCTTTTTCTTTTCACACAACTCTACTGCTGACAACTCACTGACTATCCACTTGCTCTCTTG
TGCCTTTCTGCTCTGGTTCAAGTTCCTTGATTGTTTTTGACTGCTTTTCACTGCTTTTCTTCTCACAATCCTTGCT
CAGTTCAAAGTC (SEQ ID NO: 319)
IRES647
GCCTCGGCCCCCTCACCCTCTTTTCCGGTGGCCACGCCCGGGCCACCGATACTTCCCTTCACTCCTTCGGGACTGT
TGGGGAGGAACACAACAGGGCTCCCCTGTTTTCCCATTCCTTCCCCCTTTTCCCAACCCCAACCGCCGTATCTGGT
GGCGGCAAGACACACGGGTCTTTCCCTCTAAAGCACAATTGTGTGTGTGTCCCAGGTCCTCCTGCGTACGGTGCGG
GAGTGCTCCCACCCAACTGTTGTAAGCCTGTCCAACGCGTCGTCCTGGCAAGACTATGACGTCGCATGTTCCGCTG
CGGATGCCGACCGGGTAACCGGTTCCCCAGTGTGTGTAGTGCGATCTTCCAGGTCCTCCTGGTTGGCGTTGTCCAG
AAACTGCTTCAGGTAAGTGGGGTGTGCCCAATCCCTACAAAGGTTGATTCTTTCACCACCTTAGGAATGCTCCGGA
GGTACCCCAGCAACAGCTGGGATCTGACCGGAGGCTAATTGTCTACGGGTGGTGTTTCCTTTTTCTTTTCACACAA
CTCTACTGCTGACAACTCACTGACTATCCACTTGCTCTCTTGTGCCTTTCTGCTCTGGTTCAAGTTCCTTGATTGT
TTTTGACTGCTTTTCACTGCTTTTCTTCTCACAATCCTTGCTCAGTTCAAAGTC (SEQ ID NO: 320)
IRES147
TTAAAACAGCTCTGGGGTTGTTCCCACCCCAGAGGCCCACGTGGCGGCTAGTACTCTGGTATCACGGTACCTTTGT
ACGCCTGTTTTATATCCCTTCCCCCGTAACTTAGAAGCTTTGTACCAACAAGTTCAATAGAAGGGGTACAAACCAG
TACCACCACGAACAAGCACTTCTGTTTCCCCGGTGCGATGACATAGACTGTACCCACGGTCAAAAGTCACCAATCC
GTTACCCGCTTTGGTACTTCGAGACGCCTAGTATTGCCTTGGAATTCTCGATGCGTTGCGCTCAACACTCGACCCT
GGAGTGTAGCTTAGGCTGATGAGTCTGGGCATTCCCCACCGGTGACGGTGGCCCAGGCTGCGTTGGCGGCCTACCC
ATAGCTATTGCTATGGGATGCTAGAGTTGAACAAGGTGTGAAGAGCCAATTGAGCTACTAAAGAGTCCTCCGGCCC
CTGAATGCGGCTAATCCCAACCACGGATCAAGTGCGTGCAACCCAGCACGTAGCTTGTCGTAATGCGCAAGTCTGT
GGCGGAACCGACTACTTTGGGTGTCCGTGTTTCCTTTTATTTTTATCATGGCTGCTTATGGTGACAATCTTGGATT
GTTATCATATAGCGATTGGATTGGCCATCCGGTGAGAATAAAAGATATTGTTTATCTGTTTGTGGCTTTTACTCCA
TTAAACAAAAACACACTTTCCTTAATTAAAGTGGTACTATTGATCAGATCAGCATACCACA (SEQ ID NO:
321)
IRES411
GTGTGTGTGCTCGTAATCTTGACTCCTGCCGGAATGCCGCCCGGTTCAGTGAACAAACAGCTAGGCAAGTCCCTCC
CTTCCCCTGTGGTCGGTTCTCACCGGCCACCATCCCTCCCCCAGCCTGACGTGTTACAGGCTGTGCAAAGCCCCCG
CGAAAGCTGCTCACGTGGCAATTGTGGGTCCCCCCTTTGTCAAGACACCGAGTCTTTCTCCCTTAAGGCTAGCCCG
GTCCCACGAACGTGGAACTGGCAACTAGTGGTGTCACTACACGCCTCCGACCTCGGACGCGGAGTGCTGTTCCCCA
AGCTGTAACCCTGACCCAAGACTGTGCTGCCTGGCAAGCACCGTCTGGGAAGATGTTCCGCTGTGGCTGCCAAACC
TGGTAACAGGTGCCCCAGTGTGTGTAGTCTTCCTCCAGTCTCCGGACTGGCAGTCTTGTGTAAAGATGCAGTGTAA
GGTTCAAGTGCCAAATCCCTGGAAGGAGTGACCCTCTACTGCCCTAGGAATGCTGTGCAGGTACCCCCAACTTCGG
TTGGGGATCTGAGCACAGGCTAATTGTCTACGGGTAGTTTCATTTCCCATCCTCTCTTTTTTGGCATC (SEQ ID
NO: 322)
IRES160
TTTTGCGGCTCTGCCGCCGTTCGGGTTTTACCTGTTTTCACAGAGCAAAACAGGACCTCTAGTTTCGTGCTTAAAC
GAGATCATGCTCGAACTAGAACTATAACGCTGGTCACTGGACCCGTGCCGCGCCTTGCGGATCTTTGCGGGAATGG
TGGCTAGTGGGCTGTGGAAGTGACTCTAACCACACGCCCCTCAAGTGTGGGAAAACACGAACTGGTGTAGCGACGA
CGATAGGCCTTGGGACACCCTCTCCAGTGATGGAGACCCAAGGGGCCAAAAGCCACGCCTTGTGCCCTGTCGTTCA
CAACCCCAGTGCAGTTCGTGCCAGTACCTGCTTTTGGGAAGTGTGCTTTGGACAGCTGAAAACAGTCCTAGTGGGA
GACTAAGGATGCCCAGGAGGTACCCGGAGGTAACAAGTGACACTCTGGATCTGACTTGGGGAGAGCGGGTCTGCTT
TACAGACGCCACTCTTTAAAAAACTTCTATGTCTCGTCAGGCACCGGAGGCCGGGCCTTTTCCTTTAAAACAATAC
ACTTTATGAAGACAACAATA (SEQ ID NO: 323)
IRES217
CACCCATACACCCCCACCCCCTTTTCTGTAACTCAAGTATGTGTGCTCGTAATCTTGACTCCCACGGAATGGATCG
ATCCGCTGGAGAACAAACTGCTAGATCCACATCCTCCCTCCCCTTGGGAGGACCTCGGTCCTCCCACATCCTCCCT
CCAGCCTGACGTATCACAGGCTGTGTGAAGCCCCCGCGAAAGCTGCTCACGTGGCAATTGTGGGTCCCCCCTTCAT
CAAGACACCAGGTCTTTCCTCCTTAAGGCTAGCCCCGATGTGTGAATTCACATTGGGCAACTAGTGGTGTCACTGT
GCGCTCCCAATCTCGGCCGCGGAGTGCTGTTCCCCAAGCCAAACCCCTGGCCCTTCACTATGTGCCTGGCAAGCAT
ATCTGAGAAGGTGTTCCGCTGTGGCTGCCAGCCTGGTAACAGGTGCCCCAGTGTGCGTAACCTTCTTCCGTCTCCG
GACGGTAGTGATTGGTTAAGATTTGGTGTAAGGTTCATGTGCCAACGCCCTGTGCGGGATGAAACCTCTACTGCCC
TAGGAATGCCAGGCAGGTACCCCACCTTCGGGTGGGATCTGAGCCTGGGCTAATTGTCTACGGGTAGTTTCATTTC
CAATTCTTTTATGCTGGAGTC (SEQ ID NO: 324)
IRES288
CACCCACTTTTCCGGCGGCGCACGTTCGCGTCGTCGTAAGTCTGGACTCCCAAGGCCAACTCGGTTCAACTTCGGT
TTCCGGACAAATACAAGAACCTCAGTCCTCTTGGTACTTTCTCGCCTGAGTCAACAAAGCGAGAAACCTGCCCCTC
TAACGCCAGACGAGCGGCATGAAACTTGAACTTCTGGCATGTTCCACTTTCCCTCTCCCTCACCCCACCCCACCGC
GCTCTCAAGGTCGCGCTCTTCCGGGACTAGCCCGGTTTTAAAAGTTCCTGGCACCCTTTGCCCCTCTAGGCCCTTA
AGGTAGGAACTGACCTTGTGCTGTGATCTCGGTGCGGAAGTGCTACTGCGTAGTGATTGTAAGATCCCTTTGTGGT
TCTGCCCTGGCAAGGCTACAGAGTACTGTGATCCGCTGCGGACGCCATCCTGGTAACAGGACCCCCAGTGTGCGCA
ACAGTATGTCCACGGTCTTCCGTGTTCACCACATTCGGAACACTGCTCTCGTGAAACAGTGTGTGTCCAATCCCTG
TGACCAGTATTAACCACACCACCTAGGAATGCTAGGAAGGTACCCCGGTTCGCCGGGATCTGATCCTAGGCTAATT
GTCTACAGTGGTGCTCCTTTTATTTTCCCCTCACTTCATTGACTACAACTGCTTGATTTCCGTGTTTGCTGCTCTT
CTTAGCTCTCACTGCCATTCTCAAGTGTTCACACTGTCCAAGCTCCTTTGGTTGTTTGCCTCCACTTGCCACTGTC
AACTCTTGTC (SEQ ID NO: 325)
IRES655
TTAAAACAGCTCTGGGGTTGCTCCCACCCCAGAGGCCCACGTGGCGGCTAGTAATCTGGTATCAGGTACCTTTGTA
CGCCTGTTTTATATCCCTTCCCCCGTAACTTTAGAAGCTTATCAAAAGTTCAATAGCAGGGGTACAAGCCAGTACC
TCTACGAACAAGCACTTCTGTTTCCCCGGTGAAATCATATAGACTGTACCCACGGTCAAAAGTGATTGATCCGTTA
TCCGCTTGAGTACTTCGAGAAGCCTAGTATCGCCTTGGAATCTTCGACGCGTTGCGCTCAACACTCTGCCCCGAGT
GTAGCTTAGGCTGATGAGTCTGGGCACTCCCCACCGGCGACGGTGGCCCAGGCTGCGTTGGCGGCCTACCCATGGC
TGATGCCGTGGGACGCTAGTTGTGAACAAGGTGTGAAGAGCCTATTGAGCTACTCAAGAGTCCTCCGGCCCCTGAA
TGCGGCTAATCCTAACCACGGAGCAATCGCTCACGACCCAGTGAGTAGGTTGTCGTAATGCGTAAGTCTGTGGCGG
AACCGACTACTTTGGGTGTCCGTGTTTCCCTTTATATTCATACTGGCTGCTTATGGTGACAATTTACGAATTGTTA
CCATATAGCTATTGGATTGGCCACCCAGTGCTGTGCAATATATTTGAGTGCTTCTTTCATAGGTGTTACCAACATC
ACATTTAAACCACAATAGTCAGTGCAA (SEQ ID NO: 326)
IRES726
CCTAGCCCCTTTCTGTAACGGTCTGTGTGAGACCTAAAACCACACATAATATCGTGCCTGATGGTTAACGCTTGTT
AGCCGTCTGTAAACTAAGAACCTACCAACACACTATGCACCTCTTGGCGAAGCCGCTTGGAATAAGTTGAGAGGAA
TTATGCATGCTAGTTGTGTTTGTTTACAACTAATTGTTCTAATCCAAGTGAAGCTCTTCGCTTGGGGCGGCACGAC
ACTTGCCGTAATTCTTCTACCGTCCCTCCACACCTTGTGGATGAAGGGCCGGATGTGTGGCCTCTGGCTAACCCCT
CTCTCTGGGGTGATGCTACTGGATGTTTTACTCCTAGACCAAATCACATGAACTCCTCTTGATCCACTTCGGTGGG
GCTATGAGCCTGCGGATTAATAGCTGGCGACAGCTACCCCAGGGGCCAAAAGCCACGGTGTTAGCAGCACCCTCAT
AGTCTGATGCCCAAGGGCTGATGTTGGGAGCTAGTAGTGTGTGTCTGGCCTATGTTTAGGACTTCTGGCCAAGCGC
AGAGGAGTGGGGCTGAAGGATGCCCAGAAGGTACCCGTAGGTAACCTTAAGAGACTATGGATCTGATCTGGGGCCC
CCTCACGTGGCTTTACCACGTGTTGGGGGTTAAAAAACGTCTAGGCCCCACCAGCCCACGGGAGTGGGCTTTCCCT
TAAAAAGCCCAACAATATTTATGGTGACAATTCACTGTTTCTTCTTTGCAATTTTTGTATTCTTGGACTCCTTATT
TTTTGTTTGCTTGATTTTAGTGGACATTCAGATTCAAATACAAAATGGCAGGA (SEQ ID NO: 327)
IRES639
ATTCCCAAGGCCCACTCGGTTCAACTTCGGTTTCCGGACAAATACAAAGAACCTCAGTCCTCTTGGTACTTTCTCG
CCTGAGTCTACAAAGCGAGAAACCTGCCCCTCTAACGCCAGACGAGCGGCATAAAACTCGAACTTCTGGCACGTTC
CACCACCCCTTCTCCTATCCCAACCCCCATTGCGCTCTCAAGGTCGCGCTTTTCCGAGACTAGCTCGGATTCAAAA
AGTTCCTGGCACCCTTTACCCCTTCAGGCCCTTAAGGTAGGAACTGACCTTGTGCTGTGATCTCGGTGCGGAAGTG
CTACTGCGTAGTGATTGTAAGATCCTTTTGTGGTTCTGCCCTGGCAAGGCTACAGAGTGCTGTGATCCGCTGCGGA
TGCCATCCTGGTAACAGGACCCCCAGTGTGCGCAACAGTATGTTCACGGTCTTCCGTGTTCACCACATTCGGAACA
CTGCTTTCGTGAAACAGTGTGTGTCCAATCCCTGTGATCAGTATCAACCACACCACCTAGGAATGCTAGGAAGGTA
CCCCGGTTCGCCGGGATCTGATCCTAGGCTAATTGTCTACGGTGGTGCTCCTTTTTATTTTCCACTTCAACTCACT
GGTTACAACTGCTTGATTTCTGTGTTTGCTGCTTTTCTCTGCTCTCACTGCCATTCTCAAGTGTTCACACTGTCCA
AGCTCCTTTGGTTGTTCGCTTCCACTTGCCACTGTCAACTCTTGTC (SEQ ID NO: 328)
IRES307
CCCCCCTCCCCCCCTTCCCTTCCCTTTGCAACGCAACAATTGTAAGTGCCCTCACCTGTCAATTGGGACCACCACT
TTCAGTGACCCCATGCGAAGTGCTGAGAGAAAGGAAGCTTTCTTACCCTTCATTTGTGAACCCACTGGTCTAAGCC
GCTTGGAATACGATGAGTGGAAAAGTTCATTCTTAATGGAGTGAAACATGCTTAAATTTCCAGCTCGTGCTGGTCT
TTCCAGTACGGGGCGGCCCTGTCTGGCCGTAATTCTTCAGAGTGTCACGCCACACTTGTGGATCTCACGTGCCACA
TGACAGCGCTACAGCTGGAACTGGGTGCTTGGTGCCCATGGAGTAACAGCGAAAAGTGTTAGATCAAGCCTTGCTT
GGGCTATGAGCCTGCGGAACAACAACTGGTAACAGTTGCCTCAGGGGCCGAAAGCCACGGTGTTAACAGCACCCTC
ATAGTTTGATCCACCTCAGGGTGGTGATGTTTAGCAGTTAGTAGTTGCCAATCTGTGTTCACTGAAATCTCGGCAT
ACCGTGTAGTGTACAGGGGTGAAGGATGCCCAGAAGGTACCCGTAGGTAACCTTAAGAGACTATGGATCTGATCTG
GGGCCTTGTCCGGAGTGCTTTACACACGGCTCAAGGTTAAAAAACGTCTAGCCCCACAGAGCCCGAGGGATTCGGG
TTTTCCCTTTAAAAACCCGACTAGAGCTTATGGTGACAATTATTGCTGTTCAGACGAACAGTGTAATTGTTGTCTA
TTCACAGCAGTTCTATCAGAGCTTTTCCCACAACGGATCTTCTTGGCAAGCAAATACAGCAGGAGTCAAT (SEQ
ID NO: 329)
IRES389
TTAAAACAGCCTGTGGGTTGCACCCACCCACAGGGCCCACTGGGCGCCAGCACTCTGGTATCGCGGTACCTTTGTG
CGCCTGTTTTATGACCCCTCCCCAACCGAAACTTAGAAGTTACACATACCGATCAATAGTGGGCATAGCACGCCAG
CTGTGTCTTGATCAAGCACTTCTGTTTCCCCGGACCGAGTATCAATAGACTGCTCACGCGGTTGAAGGAGAAAGCG
TTCGTTATCCGGCTAACTACTTCGAGAAACCTAGTAACACCATGAAAGTTGCGGAGTGTTTCGCTCAGCACTTCCC
CAGTGTAGATCAGGTCGATGAGTCACCGCGTTCCCCACGGGCGACCGTGGCGGTGGCTGCGCTGGCGGCCTGCCTA
TGGGATGACCCATAGGACGCTTCAACACAGACATGGTGTGAAGAGTCTATTGAGCTAGTTAGTAGTCCTCCGGCCC
CTGAATGCGGCTAATCCTAACTGCGGAGCACGTGCCCTCAAGCCAGAGGGTGGCGTGTCGTAACGGGCAACTCTGC
AGCGGAACCGACTACTTTGGGAGTCCGTGTTTCCTTTTATTCTTACACTGGCTGCTTATGGTGACAATTGAGAGAT
TGTTACCATATAGCTATTGGATTGGCCATCCGGTGAGCAACAGAGCGATTGTATACTCTTTTGTGGGTTTTGTGCC
ATTAAATCTTACTGTGATTACCACACTGAAACTCATCTTGCAGTTTAATACAGCAAA (SEQ ID NO: 330)
IRES325
TTAAAACAGCTCTGGGGTTGTTCCCACCCCAGAGGCCCACGTGGCGGCTAGTACACTGGTATTACGGTACCCTTGT
ACGCCTGTTTTATACTCCCTTCCCCGTAACTTAGAAGCTATGTAATCCAAGTTCAATAGAAGGGGGTACAAACCAG
TACCACCACGAACGAGCACTTCTGTTTCCCCGGTGAGATTGTATAAGCTGTTCCCACGGCCGAAAACGATCGATCC
GTTACCCGCTCTTGCACTTCGAGAAGCCTAGTATCATCTTGGGATCTTCGATGCGTTGCGCTCAGCACTCTACCCC
AGAGTGTAGCTTAGGTCGATGAGTCTGGACATTCCTCACCGGCGACGGTGGTCCAGGCTGCGTTGGCGGCCTACCT
GTGGTCCAAAGCCACAGGACGCTAGTTGTAAACAAGGTGTGAAGAGCCTATTGAGCTGCAAGAGAGTCCTCCGGCC
CCTGAATGCGGCTAATCCCAACCACGGAGCAGGTGGTTGCAACCCAGTAACTGGCCTGTCGTAACGCGTAAGTCTG
TGGCGGAACCGACTACTTTGGGTGTCCGTGTTTCCTTTTATTTCTATAATGGCTGCTTATGGTGACAATCATAGAT
TGTTATCATAAAGCGAATTGGATTGGCCATCCGGTGAGAGTTAAGACAATTATCCACTTGTTTGTTGGATTTACCC
CTCTGACTAATTACACACTCAACTTTATTAGGATTGTCATTCTAGTGAGATATTACCATCACA (SEQ ID NO:
331)
IRES327
TTAAAACAGCTCTGGGGTTGTTCCCACCCCAGAGGCCCACGTGGCGGCTAGTACTCCGGTATCACGGTACCCTTGT
ACGCCTGTTTTACACTCCCTTCCCCCGTAACTTAGAAGAAACAAACAAAGTTCAATAGAAGGGGGTACAAACCAGT
ACCACCACGAACAAGCACTTCTGTTTCCCCGGTGACGTTGTATAGACTGTACCCACGGTCGAAAACGATTGATCCG
TTATCCGCTTTTGTACTTCGAGAAGCCTAGTATCATCTTGGAATCTTCGATGCGTTGCGCTCAGCACTCAATCCCA
GAGTGTAGCTTAGGTCGATGAGTCTGGACGTTCCTCACCGGCGACGGTGGTCCAGGCTGCGTTGGCGGCCTACCTG
TGGCCCAAAGCCACAGGACGCTAGTTGTGAACAAGGTGTGAAGAGCCTATTGAGCTACAAGAGAGTCCTCCGGCCC
CTGAATGCGGCTAATCCTAACCACGGAGCAAGGGTGTGTGAGCCAACACATACCTTGTCGTAATGCGTAAGTTCGT
GGCGGAACCGACTACTTTGGGTGTCCGTGTTTCCTTTTATTCTTGTATTGGCTGCTTATGGTGACAATCATAGATT
GTTATCATAAAGCGACTTGGATTGGCCATCCAGTGGAAGTGAAATACCTTGTCTACCTGTCTGTTGGTTTTACTCC
ATTTGACTAATTCACTTACAAGCTTATTGCTATAATTTTTATTGACTAAACAACAATACTACA (SEQ ID NO:
332)
IRES659
CACCCTCTTTTCCGGCGGCGCATGTTCGCGTCGTCGTAAGTCTGGATTCCCAAGGCCCACTCGGTTCAACTTCGGT
TTCCGGACAAATACAAAGAACCTCAGTCCTCTTGGTACTTTCTCGCCTGAGTCTACAAAGCGAGAAACCTGCCCCT
CTAACGCCAGACGAGCGGCATAAAACTCGAACTTCTGGCACGTTCCACCACCCCTTCTCCTATCCCAACCCCCATT
GCGCTCTCAAGGTCGCGCTTTTCCGAGACTAGCTCGGATTCAAAAAGTTCCTGGCACCCTTTACCCCTTCAGGCCC
TTAAGGTAGGAACTGACCTTGTGCTGTGATCTCGGTGCGGAAGTGCTACTGCGTAGTGATTGTAAGATCCTTTTGT
GGTTCTGCCCTGGCAAGGCTACAGAGTGCTGTGATCCGCTGCGGATGCCATCCTGGTAACAGGACCCCCAGTGTGC
GCAACAGTATGTTCACGGTCTTCCGTGTTCACCACATTCGGAACACTGCTTTCGTGAAACAGTGTGTGTCCAATCC
CTGTGATCAGTATCAACCACACCACCTAGGAATGCTAGGAAGGTACCCCGGTTCGCCGGGATCTGATCCTAGGCTA
ATTGTCTACGGTGGTGCTCCTTTTTATTTTCCACTTCAACTCACTGGTTACAACTGCTTGATTTCTGTGTTTGCTG
CTTTTCTCTGCTCTCACTGCCATTCTCAAGTGTTCACACTGTCCAAGCTCCTTTGGTTGTTCGCTTCCACTTGCCA
CTGTCAACTCTTGTC (SEQ ID NO: 333)
IRES693
AACAGCCTGTGGGTTGTACCCACCCACAGGGCCCACTGGGCGCTAGCACACTGGTATCACGGTACCTTTGTGCGCC
TGTTTTATACCCCCTACCCCAACTTTAAACTTAGAAGCAAAGCAAACCCGATCAATAGCAGATGTAGCAAGCCAGT
TGCATCTTGATCAAGTACTTCTGTCTCCCCGGACTGAGTATCAATAGACTGCTCACGCGGTTGAAGGAGAAAACGT
TCGTTACCCGACCAACTACTTCGAGAAACCTAGTAACACCATGAAAGTAGCAGGGTGTTTCGATCAGCACAACCCC
AGTGTAGATCAGGCTGATGAGTCACCGCATCCCCCACGGGCGACCGTGGCGGTGGCTGCGCTGGCGGCCTGCCCAT
GGGGCAACCCATGGGACGCTCTAATATGGACATGGTGTGAAGAGTCTATTGAGCTAGTTGGTAGTCCTCCGGCCCC
TGAATGCGGCTAATCCTAACTGCGGAGCACATACCCCAAAGCCAAGGGGCAGTGTGTCGTAACGGGCAACTCTGCA
GCGGAACCGACTACTTTGGGTGTCCGTGTTTCCTTTTATTCTCAAACTGGCTGCTTATGGTGACAATCTCAGAGTT
GTTACCATATAGCTATTGGATTGGCCATCCGGTGAGCAACAGAGCAATTGTCTACCAATTTGTTGGATTTATTCCA
CTCAATCTAGCGGTTTTCAGAACATTGAATTATATTCTAACCCTCAACAAGACGAA (SEQ ID NO: 334)
IRES772
TGGAATTAACATCATTTCCGACGAAAGTGCTATCATGCCTCCCCGATTGTGTGATGTTTTCTGCCCTGCTGGGCGG
AGCATTCTCGGGTTGAGAAACCTTGAATCTTTTCCTTTGGAACCTTGGTTCCCCCGGTCTAAGCCGCTTGGAATAT
GACAGGGTTATTTTCCTTATCTTACTTCTACTTTCACGGGTTCTATCCGTGAAAAGGGTACGTGTTGCCCCTTCCT
TCTTCGGAGAATTCACACGGCGGTCTTTCCGTCTCTCGACAGGTGTGAATGCAACATGCCGGAAACGGTGAAGAAA
ACAGTTTTCTGCGGAAGTCTTGAGTGCGCATCGAAACAGCTGTAGCGACCTCACAGTAGCAGCGGACTCCCCTCTT
GGTGACAAGAGCCTCTGCGGCCAAAAGCCCCGTGGATAAGATCCACTGCTGTGAGCGGTGCAACCCCAGCACCCTG
ATTCGATGAACATTCTTTACGGAACCAGAAGATGGTTTTCTTAAGCCCTCCGGTAGAGAAGCCAAGAATGTCCTGA
AGGTACCCCGCGTGCGGGATCTGATCAGGAGACCAATTGACTGTGCTTTACACTGTCACTTTGGTTTAAAAACTGT
CACAGCTTCTCCAAACCAAGTGGTCTTGGTTTTCCAATTTTACTAACTGGCAAT (SEQ ID NO: 335)
IRES537
TTACCCGTATTCCTTCCCTTCCCCCGCAACCACCACGCTTACTCGCGCACGTGTTGAGTGGCACGTGCGTTGTCCA
AACAGCTACACCCACACCCTTCGGGGGGGGTTTGTCCCGCCCTCGGGTTCCTCGCGGAACCCCCCCCTCCCTCTCT
CTCTTTCTATCCGCCCTCACTTCCCATAACTACAGTGCTTTGGTAGGTGAGCACCCTGACCCCCCGCGGAAGCTGC
TAACGTGGCAACTGTGGGGATCCAGGCAGGTTATCAAAGGCACCCGGTCTTTCCGCCTTCAGGAGTATCTCTGCCG
GTGAATTCCGGTAGGGCTCTGCTTGGTGCCAACCTCCCCCAAATGCGCGCTGCGGGAGTGCTCTTCCCCAACTCAT
CTTAGTAACCTCTCATGTGTGTGCTTGGTCAGCATATCTGAGGCGACGTTCCGCTGTCCCAGACCAGTCCAGCAAT
GGACGGGCCAGTGTGCGTAGTCGCTTTCCGGTTTTCCGGCGCATGTTTGGCGAAACGCTGAGGTAAGGTTGGTGTG
CCCAACGCCCGTAATTTGGTGATACCTCAAGACCACCCAGGAATGCCAGGGAGGTACCCCACTTCGGTGGGATCTG
ACCCTGGGCTAATTGTCTACGGTGGTTCTTCTTGCTTCCACTTCTCTTTTTTCTGGC (SEQ ID NO: 336)
IRES703
AACAGCCTGTGGGTTGTACCCACCCACAGGGCCCACTGGGCGCCAGCACTCTGGTATTACGGTACCCTTGTGCGCC
TGTTTTATATCCCTTCCCCAAGGTAAACGTTAGAAGCAATGCACTCCCGATCAATAGAAGGTGCGACGCGCCAGTC
GTATCTCGATCAAGCACTTCTGTTTCCCCGGACCGAGTATCAATAGACTGCTAGCGCGGTTGAAGGAGAAAACGTT
CGTTACCCGACTAACTACTTCGAGAAGCCCAGTAGCGCCATGAAAGTTGCAGAGTGTTTCGCTCAGCACTTCCCCC
GTGTAGATCAGGCCGATGAGTCACCGCAATCCCCACGGGCGACCGTGGCGGTGGCTGCGTTGGCGGCCTGCCTATG
GGGAAACCCATAGGACGCTCAAATACAGACATGGTGCGAAGAGTCTATTGAGCTAGTTAGTACTCCTCCGGCCCCT
GAATGCGGCTAATCCTAACTGCGGAGCAAGTACTCACAATCCAGTGGGCAGCTTGTCGTAACGGGTAACTCTGCAG
CGGAACCGACTACTTTGGGTGTCCGTGTTTCCTTTTATTCCTACATTGGCTGCTTATGGTGACAATCAAAGAGTTG
TTACCATATAGCTATTGGATTGGCCATCCGGTGACTAATAAAGCAATTATATTTCTTTTTGTTGGGTTTGTACCCC
TTAATACAACCAGCTTCACCACATTGTGTTACATCATTAGACTGAACAAGGCAAA (SEQ ID NO: 337)
IRES758
TCTTTTTGACGTGGTTGGAATTAACATCATTTCCGACGAAAGTGCTATCATGCCTCCCCGATTGTGTGATGTTTTC
TGCCCTGCTGGGCGGAGCATTCTCGGGTTGAGAAACCTTGAATCTTTTCCTTTGGAACCTTGGTTCCCCCGGTCTA
AGCCGCTTGGAATATGACAGGGTTATTTTCCTTATCTTACTTCTACTTTCACGGGTTCTATCCGTGAAAAGGGTAC
GTGTTGCCCCTTCCTTCTTCGGAGAATTCACACGGCGGTCTTTCCGTCTCTCGACAGGTGTGAATGCAACATGCCG
GAAACGGTGAAGAAAACAGTTTTCTGCGGAAGTCTTGAGTGCGCATCGAAACAGCTGTAGCGACCTCACAGTAGCA
GCGGACTCCCCTCTTGGTGACAAGAGCCTCTGCGGCCAAAAGCCCCGTGGATAAGATCCACTGCTGTGAGCGGTGC
AACCCCAGCACCCTGATTCGATGAACATTCTTTACGGAACCAGAAGATGGTTTTCTTAAGCCCTCCGGTAGAGAAG
CCAAGAATGTCCTGAAGGTACCCCGCGTGCGGGATCTGATCAGGAGACCAATTGACTGTGCTTTACACTGTCACTT
TGGTTTAAAAACTGTCACAGCTTCTCCAAACCAAGTGGTCTTGGTTTTCCAATTTTACTAACTGGCAAT (SEQ
ID NO: 338)
IRES753
TTTGAAAAGGGGGTGGGGGGGCTTCGGCCCCCTCACCCTCTTTTCCGGCGGCGCATGTTCGCGTCGTCGTAAGTCT
GGATTCCCAAGGCCCACTCGGTTCAACTTCGGTTTCCGGACAAATACAAAGAACCTCAGTCCTCTTGGTACTTTCT
CGCCTGAGTCTACAAAGCGAGAAACCTGCCCCTCTAACGCCAGACGAGCGGCATAAAACTCGAACTTCTGGCACGT
TCCACCACCCCTTCTCCTATCCCAACCCCCATTGCGCTCTCAAGGTCGCGCTTTTCCGAGACTAGCTCGGATTCAA
AAAGTTCCTGGCACCCTTTACCCCTTCAGGCCCTTAAGGTAGGAACTGACCTTGTGCTGTGATCTCGGTGCGGAAG
TGCTACTGCGTAGTGATTGTAAGATCCTTTTGTGGTTCTGCCCTGGCAAGGCTACAGAGTGCTGTGATCCGCTGCG
GATGCCATCCTGGTAACAGGACCCCCAGTGTGCGCAACAGTATGTTCACGGTCTTCCGTGTTCACCACATTCGGAA
CACTGCTTTCGTGAAACAGTGTGTGTCCAATCCCTGTGATCAGTATCAACCACACCACCTAGGAATGCTAGGAAGG
TACCCCGGTTCGCCGGGATCTGATCCTAGGCTAATTGTCTACGGTGGTGCTCCTTTTTATTTTCCACTTCAACTCA
CTGGTTACAACTGCTTGATTTCTGTGTTTGCTGCTTTTCTCTGCTCTCACTGCCATTCTCAAGTGTTCACACTGTC
CAAGCTCCTTTGGTTGTTCGCTTCCACTTGCCACTGTCAACTCTTGTCATGGACCCCTCAAAC (SEQ ID NO:
339)
IRES650
TTAAAACAGCCTGTGGGTTGTACCCACCCACAGGGCCCACTGGGCGCTAGCACACTGGTATCACGGTACCTTTGTG
CGCCTGTTTTATACCCCCTACCCCAACTTTAAACTTAGAAGCAAAGCAAACCCGATCAATAGCAGATGTAGCAAGC
CAGTTGCATCTTGATCAAGTACTTCTGTCTCCCCGGACTGAGTATCAATAGACTGCTCACGCGGTTGAAGGAGAAA
ACGTTCGTTACCCGACCAACTACTTCGAGAAACCTAGTAACACCATGAAAGTAGCAGGGTGTTTCGATCAGCACAA
CCCCAGTGTAGATCAGGCTGATGAGTCACCGCATCCCCCACGGGCGACCGTGGCGGTGGCTGCGCTGGCGGCCTGC
CCATGGGGCAACCCATGGGACGCTCTAATATGGACATGGTGTGAAGAGTCTATTGAGCTAGTTGGTAGTCCTCCGG
CCCCTGAATGCGGCTAATCCTAACTGCGGAGCACATACCCCAAAGCCAAGGGGCAGTGTGTCGTAACGGGCAACTC
TGCAGCGGAACCGACTACTTTGGGTGTCCGTGTTTCCTTTTATTCTCAAACTGGCTGCTTATGGTGACAATCTCAG
AGTTGTTACCATATAGCTATTGGATTGGCCATCCGGTGAGCAACAGAGCAATTGTCTACCAATTTGTTGGATTTAT
TCCACTCAATCTAGCGGTTTTCAGAACATTGAATTATATTCTAACCCTCAACAAGACGAA (SEQ ID NO:
340)
*Because the sequences are RNA sequences, it is understood that the T′s represent uracil in the sequences of this table.

### Untranslated Region

In some embodiments, the circRNA comprises one or more untranslated regions (UTR). In some embodiments, the circRNA comprises a 5′ UTR, a 3′ UTR or both a 5′ UTR and a 3′ UTR.

In some embodiments, the one or more UTR comprises a CAA40 comprising SEQ ID NO: 41 or a pAC18 comprising SEQ ID NO: 42.

In some embodiments, one or more UTR comprises any one of SEQ ID NO: 41-45, 46, 47 or 272. In some embodiments, the one or more UTR comprises SEQ ID NO: 47 or SEQ ID NO: 272.

As shown in Table 2A below, as used herein, SEQ ID NO: 43 and SEQ ID NO: 265 refer to the same sequence and are interchangeable throughout the specification. As used herein, SEQ ID NO: 46 and SEQ ID NO: 272 refer to the same 3′ UTR sequence and are interchangeable throughout the specification.

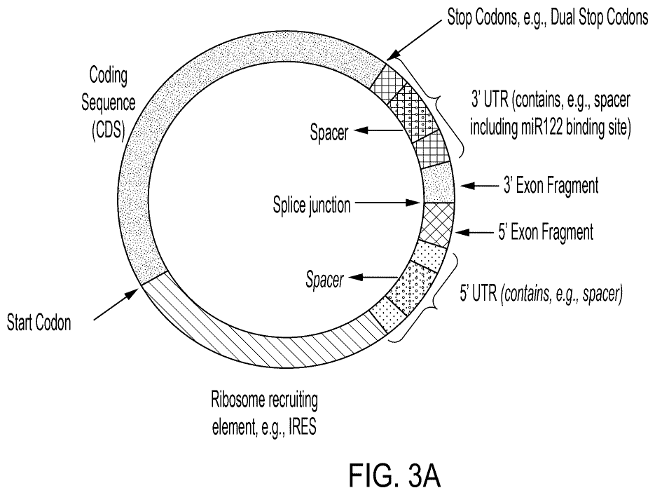

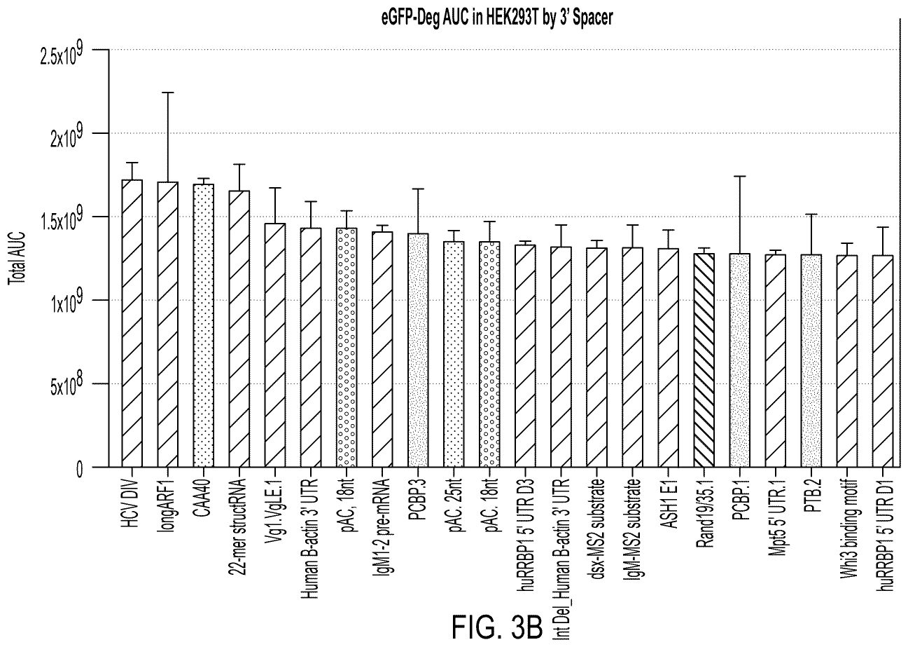

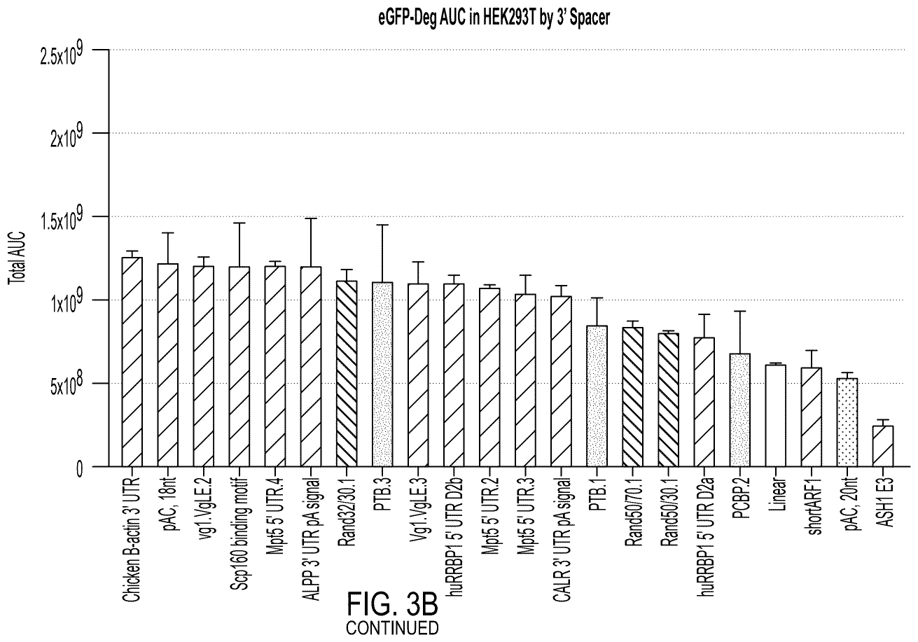

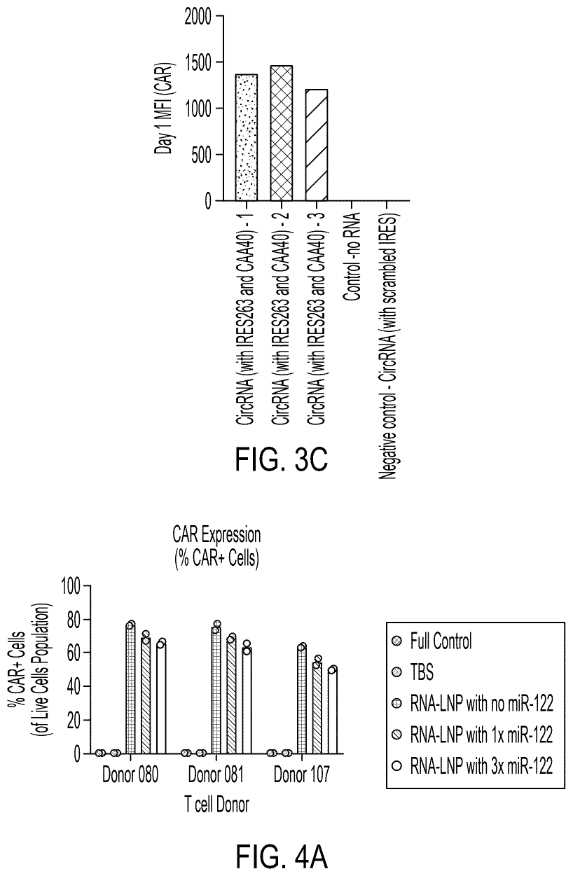

In some embodiments, the 3′ UTR comprises a 3′ exon fragment. In some embodiments, the 3′ UTR comprises a 3′ exon fragment as shown in FIG. 3A. In some embodiments, the 3′ UTR comprises a 3′ exon fragment from Twort. In some embodiments, the 3′ UTR comprises a 3′ exon fragment from a Group I intron. In some embodiments, the 3′ UTR comprises a 3′ exon fragment from a Group II intron.

In some embodiments, the 3′ UTR comprises SEQ ID NO: 268 or SEQ ID NO: 272. In some embodiments, the 3′ UTR comprises SEQ ID NO: 358 or SEQ ID NO: 362. It is understood that SEQ ID NO: 358 is identical to SEQ ID NO: 268 with Ts replaced by Us. It is understood that SEQ ID NO: 362 is identical to SEQ ID NO: 272 with Ts replaced by Us. It is understood that in the context of circRNA, SEQ ID NO: 268 is equivalent to SEQ ID NO: 358 and comprises uracils in place of thymines, and may be interchangeably used herein. Similarly, in the context of RNA, SEQ ID NO: 362 is equivalent to SEQ ID NO: 272 and comprises uracils in place of thymines, and may be interchangeably used herein.

In some embodiments, the 3′ UTR does not comprise a 3′ exon fragment. In some embodiments, the 3′ UTR does not comprise a 3′ exon fragment as shown in FIG. 3A. In some embodiments, the 3′ UTR does not comprise a 3′ exon fragment from Twort. In some embodiments, the 3′ UTR does not comprise a 3′ exon fragment from a Group I intron. In some embodiments, the 3′ UTR does not comprise a 3′ exon fragment from a Group II intron.

In some embodiments, the 3′ UTR comprises

(SEQ ID NO: 349)
TAATAGaacaaacacaaacCAAACACCATTGTCACACTCCAaacaacaaacaaacaACCACACAAA
TGGTCGCCGA.

In some embodiments, the 3′ UTR comprises

(SEQ ID NO: 350)
TAATAGaaaaacacaaacCAAACACCATTGTCACACTCCAaaaaaaacaaacaACCACACAAATG
GTCGCCGA.

In some embodiments, the 3′ UTR comprises SEQ ID NO: 359. In the context of RNA, it is understood that SEQ ID NO: 359 is equivalent to SEQ ID NO: 349 and comprises uracils in place of thymines, and may be interchangeably used herein.

In some embodiments, the 3′ UTR comprises SEQ ID NO: 363. In the context of RNA, it is understood that SEQ ID NO: 363 is equivalent to SEQ ID NO: 350 and comprises uracils in place of thymines, and may be interchangeably used herein.

In some embodiments, the 3′ UTR comprises a single microRNA binding site.

In some embodiments, the 5′ UTR comprises a 5′ exon fragment. In some embodiments, the 5′ UTR comprises a 5′ exon fragment as shown in FIG. 3A. In some embodiments, the 5′ UTR comprises a 5′ exon fragment from Twort. In some embodiments, the 5′ UTR comprises a 5′ exon fragment from a Group I intron. In some embodiments, the 5′ UTR comprises a 5′ exon fragment from a Group II intron.

In some embodiments, the 5′ UTR comprises SEQ ID NO: 265.

In some embodiments, the 5′ UTR comprises SEQ ID NO: 352. In the context of RNA, it is understood that SEQ ID NO: 352 is equivalent to SEQ ID NO: 265 and comprises uracils in place of thymines, and may be interchangeably used herein.

In some embodiments, the 5′ UTR does not comprise a 5′ exon fragment. In some embodiments, the 5′ UTR does not comprise a 5′ exon fragment as shown in FIG. 3A. In some embodiments, the 5′ UTR does not comprise a 5′ exon fragment from Twort. In some embodiments, the 5′ UTR does not comprise a 5′ exon fragment from a Group I intron. In some embodiments, the 5′ UTR does not comprise a 5′ exon fragment from a Group II intron.

In some embodiments, the 5′ UTR comprises

(SEQ ID NO: 348)
TCGGCGACCATTTGTGTGGTGAGCTCTAAGTGTTGATCAGCGGGCGCAG
CTGCATATGACCTCTGCGTGTGCCTGGTAATTGACTAAGATATC.

In some embodiments, the 5′ UTR comprises SEQ ID NO: 353. In the context of RNA, it is understood that SEQ ID NO: 353 is equivalent to SEQ ID NO: 348 and comprises uracils in place of thymines, and may be interchangeably used herein.

In some embodiments, the 5′ UTR comprises SEQ ID NO: 348 and the 3′ UTR comprises SEQ ID NO: 349. In some embodiments, the 5′ exon fragment comprises SEQ ID NO: 382 and a 3′ exon fragment comprises SEQ ID NO: 389.

In some embodiments, the 5′ UTR comprises SEQ ID NO: 353 and the 3′ UTR comprises SEQ ID NO: 359.

In some embodiments, the 5′ UTR comprises SEQ ID NO: 348 and the 3′ UTR comprises SEQ ID NO: 350. In some embodiments, the 5′ UTR comprises SEQ ID NO: 353 and the 3′ UTR comprises SEQ ID NO: 363.

TABLE 2A
Exemplary UTR Sequences*

CAA40
ACAACAACAAACAACAAAACAACAAACAACACAAAACCAA (SEQ ID NO: 41)
pAC18
AAAAACAAAAAACAAAAACCACACAAATGGTCGCCGA (SEQ ID NO: 42)
Exemplary 5′ UTR
5′ UTR_1
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATC (SEQ ID NO: 43)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATC (SEQ ID NO: 265)
5′ UTR_2
TCGGCGACCATTTGTGTGGTGAGCTCTAAGTGTTGATCAGCGGGCGCAGCTGCATATGACCTCTGCGTGTGCCTGG
TAATTGACTAAGATATC (SEQ ID NO: 348)
Exemplary 3′ UTR
3′ UTR_1
TAATAGAAAAACAAAAAACAAAAACCACACAAATGGTCGCCGAAACTACTGAAAGCAT (SEQ ID NO: 44)
3′ UTR_2
TGATAATAGAAAAACAAAAAACAAAAACCACACAAATGGTCGCCGAAACTACTGAAAGCAT (SEQ ID NO: 45)
3′ UTR_3
TAATAGAAAAACACAAACCAAACACCATTGTCACACTCCAAAAAAAACAAACAACCACACAAATGGTCGCCGAAAC
TACTGAAAGCAT (SEQ ID NO: 46)
TAATAGAAAAACACAAACCAAACACCATTGTCACACTCCAAAAAAAACAAACAACCACACAAATGGTCGCCGAAAC
TACTGAAAGCAT (SEQ ID NO: 272)
3′ UTR_4
TAATAGAAAAACACAAACCAAACACCATTGTCACACTCCAACTCATGATCAGTACAGAACAAACACCATTGTCACA
CTCCAAGCTGACATACATATCATAACTCTACTATAGACAAACACCATTGTCACACTCCAAAAAAAACAAACAACCA
CACAAATGGTCGCCGAAACTACTGAAAGCAT (SEQ ID NO: 47)
3′ UTR_5
TAATAGaacaaacacaaacCAAACACCATTGTCACACTCCAaacaacaaacaaacaACCACACAAATGGTCGCCGAa
actactgaaagcat (SEQ ID NO: 268)
3′ UTR_6
TAATAGAACAAACACAAACCAAACACCATTGTCACACTCCAAACAACAAACAAACAACCACACAAATGGTCGCCGA
AACTACTGAAAGCAT (SEQ ID NO: 271)
3′ UTR_7
TAATAGaacaaacacaaacCAAACACCATTGTCACACTCCAaacaacaaacaaacaACCACACAAATGGTCGCCGA
(SEQ ID NO: 349).
3′ UTR_8
TAATAGaaaaacacaaacCAAACACCATTGTCACACTCCAaaaaaaacaaacaACCACACAAATGGTCGCCGA
(SEQ ID NO: 350).
*Because the sequences are RNA sequences, it is understood that the T′s represent uracil in the sequences of this table.

### 5′ and 3′ Spacers

In some embodiments, the nucleic acid molecule for making a circular RNA may comprise a 5′ UTR comprising a 5′ spacer.

The 5′ spacer sequence comprises a random sequence that increases circularization efficiency. The 5′ spacer sequence may be of any length (e.g., 10 to 100 nucleotides, 10 to 90 nucleotides, 10 to 80 nucleotides, 10 to 70 nucleotides, 10 to 60 nucleotides, 10 to 50 nucleotides, 10 to 40 nucleotides, 10 to 30 nucleotides, 10 to 20 nucleotides, 20 to 100 nucleotides, 20 to 90 nucleotides, 20 to 80 nucleotides, 20 to 70 nucleotides, 20 to 60 nucleotides, 20 to 50 nucleotides, 20 to 40 nucleotides, 20 to 30 nucleotides, 30 to 100 nucleotides, 30 to 90 nucleotides, 30 to 80 nucleotides, 30 to 70 nucleotides, 30 to 60 nucleotides, 30 to 50 nucleotides, 30 to 40 nucleotides, 40 to 100 nucleotides, 40 to 90 nucleotides, 40 to 80 nucleotides, 40 to 70 nucleotides, 40 to 60 nucleotides, 40 to 50 nucleotides, 50 to 100 nucleotides, 50 to 90 nucleotides, 50 to 80 nucleotides, 50 to 70 nucleotides, 50 to 60 nucleotides, 60 to 100 nucleotides, 60 to 90 nucleotides, 60 to 80 nucleotides, 60 to 70 nucleotides, or 50 nucleotides). For example, in some embodiments, the length of the 5′ spacer is selected to optimize translation of the protein-coding nucleic acid sequence.

In some embodiments, the 5′ spacer sequence is between 20 and 50 nucleotides in length. In some embodiments, the 5′ spacer sequence is between 30 and 100 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 37, 38, 39, 40, 41, 42, 43, 44, 45, 46, 47, 48, 49, 50, 55, 60, 65, 70, 75, 80, 85, 90, 95, or 100 nucleotides in length. In some embodiments, the 5′ spacer sequence includes a 5′ inner homology element. As used herein, the terms “internal homology region” and “inner homology element (IHE)” are used interchangeably. In some embodiments, the internal homology element is about 5-50 nucleotides in length. In some embodiments, the internal homology element is about 5-30 nucleotides in length. In some embodiments, the internal homology region is about 10-25 nucleotides in length. In some embodiments, the internal homology element is 5, 6, 7, 8, 9, 10, 11, 12, 13, 14, 15, 16, 17, 18, 19, or 20 nucleotides in length. In some embodiments, the 5′ internal homology element is located at the 5′ end of the 5′ spacer sequence. The internal homology element forms base-pairing with an internal homology region, e.g., at the 3′ end (3′ inner homology element). In some embodiments, the nucleic acid described herein comprising 5′ and 3′ inner homology elements that are 75%, 80%, 85%, 90%, 95%, or 100% complementary to each other.

In some embodiments, the 5′ spacer sequence comprises a polyA sequence. The polyA sequence may comprise 15-30 As. In some embodiments, the polyA sequence comprises 20, 21, 22, 23, 24, 25, 26, 27, 28, 29 or 30 As. In other embodiments, the 5′ spacer sequence comprises a polyAC sequence.

In some embodiments, the nucleic acid molecule for making a circular RNA comprises a 3′ UTR comprising a 3′ spacer. In some embodiments, the 3′ spacer comprises a microRNA binding site. In some embodiments, the circRNA comprises UTRs comprising a spacer sequence, or variants thereof, selected from Table 2B.

In some aspects, provided herein is a circular ribonucleic acid (circRNA) comprising: (a) a coding sequence encoding a chimeric antigen receptor (CAR) comprising an anti-CD19 antibody or antigen-binding fragment thereof, (b) an Internal Ribosome Entry Site (IRES) comprising a sequence at least 85% identical to any one of SEQ ID NO: 1-11, (c) 3′ and 5′ untranslated region(s) (UTR), (d) 3′ and 5′ exon fragments, and (e) a single microRNA binding site.

In some embodiments, the 3′ UTR comprises the single microRNA binding site.

In some embodiments, the microRNA binding site comprises SEQ ID NO: 40.

In some embodiments, the single microRNA binding site is flanked by an upstream spacer and a downstream spacer, and wherein the sequence of each of the upstream spacer and the downstream spacer has a hydropathy index between −0.6 to −0.3.

In some embodiments, the sequence of each of the upstream spacer and the downstream spacer has a hydropathy index between −0.6 to −0.3. In some embodiments, the sequence of each of the upstream spacer and the downstream spacer has a hydropathy index between −0.6 to −0.4. In some embodiments, the sequence of each of the upstream spacer and the downstream spacer has a hydropathy index between −0.6 to −0.5.

In some embodiments, the sequence of the upstream spacer has a hydropathy index of −0.6 to −0.4. In some embodiments, the sequence of the upstream spacer has a hydropathy index of −0.6 to −0.5.

In some embodiments, the miR-122_2 upstream spacer has a hydropathy index of −0.59. In some embodiments, the miR-122_2 downstream spacer has a hydropathy index of −0.53. In some embodiments, the miR-122_1 upstream spacer has a hydropathy index of −0.5. In some embodiments, the miR-122_1 downstream spacer has a hydropathy index of −0.346. In contrast, a 15 nucleotide poly A sequence of consecutive adenines has a hydropathy index of −0.1, while a 15 nucleotide poly C sequence of consecutive cytosines has a hydropathy index of −1.7. A 16 nucleotide sequence of alternating cytosines and adenines has a hydropathy index of −0.9.

In some aspects, provided herein is a circular ribonucleic acid (circRNA) comprising: (a) a 5′ exon fragment and a 5′ untranslated region (UTR), (b) an Internal Ribosome Entry Site (IRES), (c) a coding sequence encoding a chimeric antigen receptor (CAR) comprising an anti-CD19 antibody or antigen-binding fragment thereof, and (d) a 3′ UTR comprising an upstream spacer, a microRNA binding site, and a downstream spacer, and a 3′ exon fragment, wherein each of the upstream spacer and downstream spacer comprise multiple instances of consecutive adenines (As) but do not comprise more than five (5) consecutive adenines (As).

In some aspects, provided herein is a circular ribonucleic acid (circRNA) comprising, in order: (a) a 5′ exon fragment and a 5′ untranslated region (UTR), (b) an Internal Ribosome Entry Site (IRES), (c) a coding sequence encoding a chimeric antigen receptor (CAR) comprising an anti-CD19 antibody or antigen-binding fragment thereof, and (d) a 3′ UTR comprising an upstream spacer, a microRNA binding site, and a downstream spacer, and a 3′ exon fragment, wherein each of the upstream spacer and downstream spacer comprise multiple instances of consecutive adenines (As) but do not comprise more than five (5) consecutive adenines (As).

In some embodiments, each of the upstream spacer and downstream spacer include consecutive adenines but do not comprise more than five (5) consecutive adenines (As). In some embodiments, each of the upstream spacer and the downstream spacer comprise 2, 3, 4 or 5 consecutive adenines.

In some embodiments, each of the upstream spacer and downstream spacer include consecutive adenines but do not comprise more than four (4) consecutive adenines (As). In some embodiments, each of the upstream spacer and the downstream spacer comprise 2, 3 or 4 consecutive adenines.

In some embodiments, each of the upstream spacer and downstream spacer include consecutive adenines but do not comprise more than three (3) consecutive adenines (As). In some embodiments, each of the upstream spacer and the downstream spacer comprise 2 or 3 consecutive adenines.

In some embodiments, the upstream spacer comprises 2, 3, 4 or 5 consecutive adenines. In some embodiments, the upstream spacer comprises two consecutive adenines. In some embodiments, the upstream spacer comprises three consecutive adenines. In some embodiments, the upstream spacer comprises four consecutive adenines. In some embodiments, the upstream spacer comprises five consecutive adenines.

In some embodiments, the downstream spacer comprises 2, 3, 4 or 5 consecutive adenines. In some embodiments, the downstream spacer comprises two consecutive adenines. In some embodiments, the downstream spacer comprises three consecutive adenines. In some embodiments, the downstream spacer comprises four consecutive adenines. In some embodiments, the downstream spacer comprises five consecutive adenines.

In some embodiments, each of the upstream spacer and downstream spacer comprise one or more instances of a cytosine (C) interrupting consecutive As.

In some embodiments, the upstream spacer and downstream spacer facilitate circularization. In some embodiments, the circularization is increased by 5%, 6%, 7%, 8%, 9%, 10% or more by interrupting a poly A sequence comprising greater than five consecutive adenines in the upstream and/or downstream spacer by inserting a cytosine.

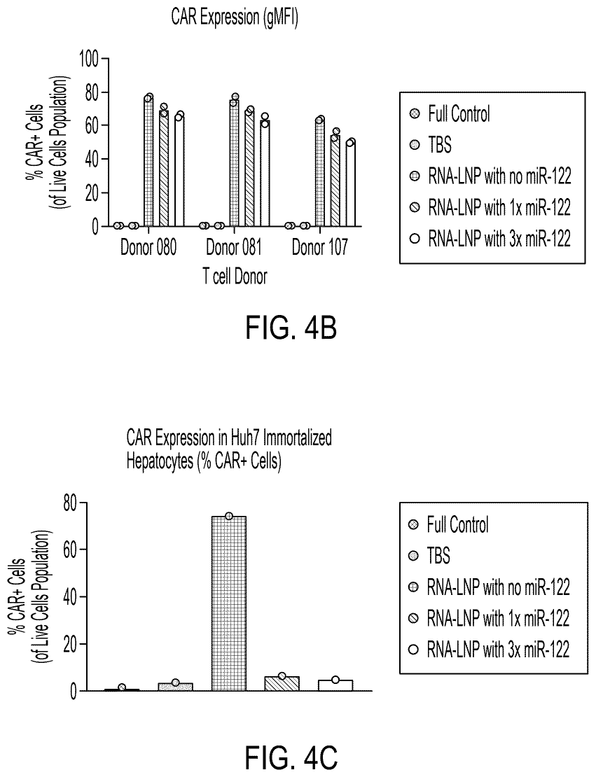

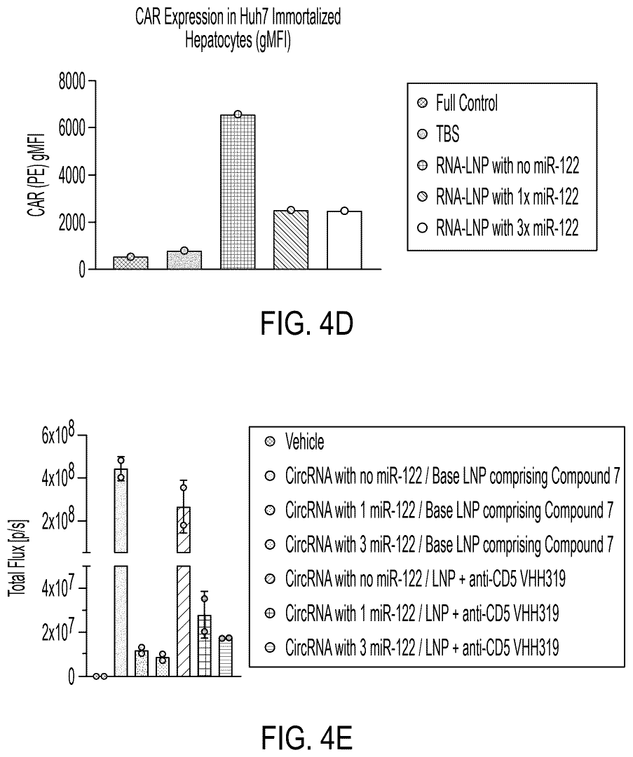

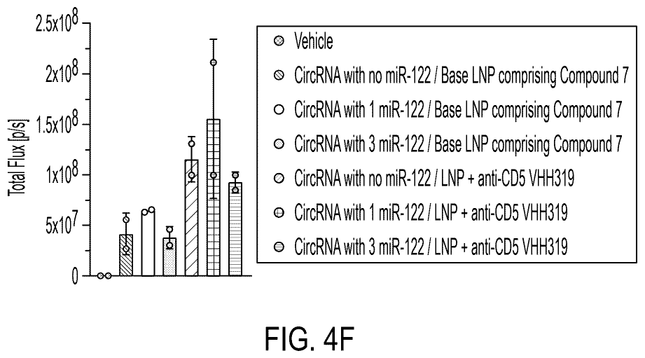

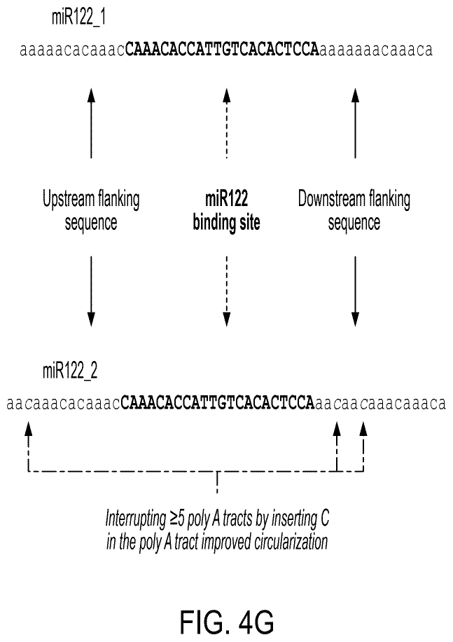

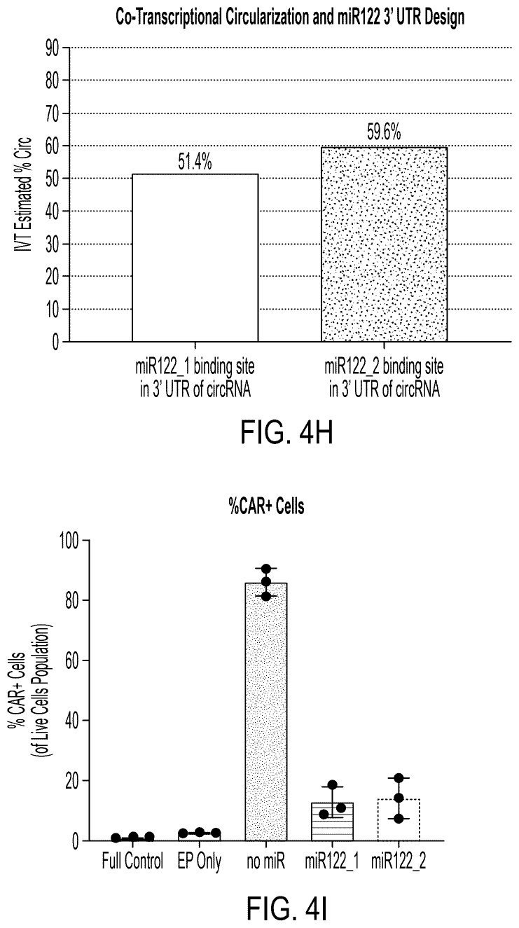

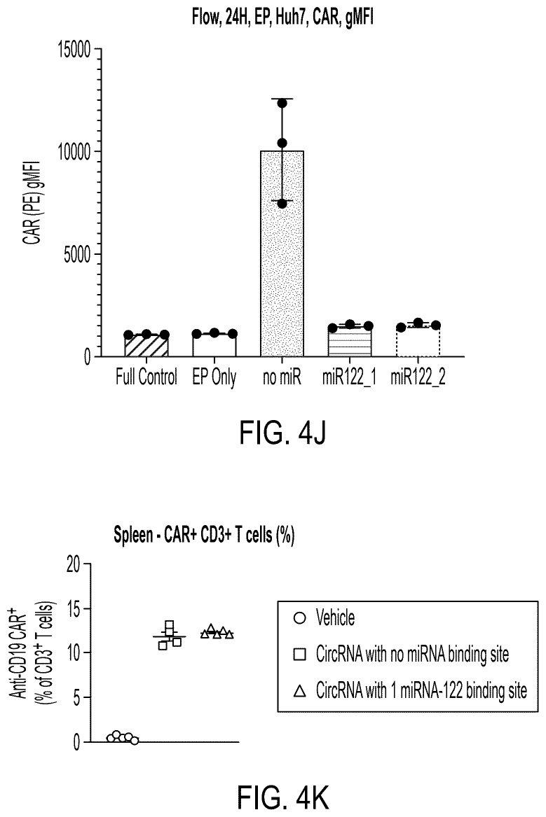

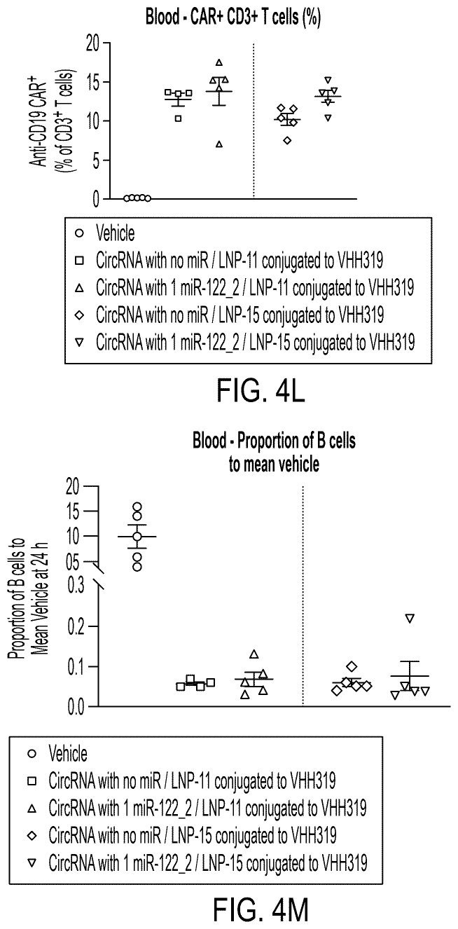

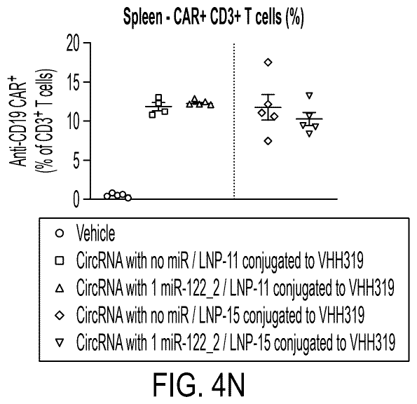

As a non-limiting example, the miR-122_2 redesigned spacer where polyA sequences were interrupted by inserting a cytosine increased circularization by about 8.2% relative to a miR-122_1 spacer (FIG. 4G and FIG. 4H).

In some aspects, provided herein is a circular ribonucleic acid (circRNA) comprising: a 5′ untranslated region (UTR), an Internal Ribosome Entry Site (IRES), a coding sequence encoding a chimeric antigen receptor (CAR) comprising an anti-CD19 antibody or antigen-binding fragment thereof, and a 3′ untranslated region (UTR) comprising a microRNA binding site flanked by an upstream spacer and a downstream spacer, wherein each of the upstream spacer and downstream spacer comprise multiple instances of consecutive adenines (As) but do not comprise more than five (5) consecutive adenines (As). In some embodiments, each of the upstream spacer and the downstream spacer comprise 2, 3, 4 or 5 consecutive adenines.

In some embodiments, each of the upstream spacer and downstream spacer comprise no uracil.

In some embodiments, the upstream spacer sequence and the downstream spacer sequence consist only of As and Cs.

In some embodiments, each of the upstream spacer and downstream spacer have a length of not more than 18 nucleotides. In some embodiments, each of the upstream spacer and downstream spacer have a length of 12 nucleotides. In some embodiments, each of the upstream spacer and downstream spacer have a length of 13 nucleotides. In some embodiments, each of the upstream spacer and downstream spacer have a length of 14 nucleotides. In some embodiments, each of the upstream spacer and downstream spacer have a length of 15 nucleotides. In some embodiments, each of the upstream spacer and downstream spacer have a length of 16 nucleotides. In some embodiments, each of the upstream spacer and downstream spacer have a length of 17 nucleotides. In some embodiments, each of the upstream spacer and downstream spacer have a length of 18 nucleotides.

In some embodiments, each of the upstream spacer and downstream spacer have a length of 12-18 nucleotides. In some embodiments, each of the upstream spacer and downstream spacer have a length of 12, 13, 14, 15, 16, 17 or 18 nucleotides. In some embodiments, each of the upstream spacer and downstream spacer have a length of 12 nucleotides. In some embodiments, each of the upstream spacer and downstream spacer have a length of 13 nucleotides. In some embodiments, each of the upstream spacer and downstream spacer have a length of 14 nucleotides. In some embodiments, each of the upstream spacer and downstream spacer have a length of 15 nucleotides. In some embodiments, each of the upstream spacer and downstream spacer have a length of 16 nucleotides. In some embodiments, each of the upstream spacer and downstream spacer have a length of 17 nucleotides. In some embodiments, each of the upstream spacer and downstream spacer have a length of 18 nucleotides.

In some embodiments, each of the upstream spacer and downstream spacer have a length of 12-15 nucleotides.

In some embodiments, the upstream spacer and the downstream spacer do not differ in length by more than 4 nucleotides.

In some embodiments, the upstream spacer and the downstream spacer do not differ in length by more than 3 nucleotides.

In some embodiments, the upstream spacer and the downstream spacer do not differ in length by more than 2 nucleotides.

In some embodiments, the upstream spacer and the downstream spacer do not differ in length by more than 1 nucleotide.

In some embodiments, the microRNA binding site is a microRNA-122 (miR-122) binding site.

TABLE 2B
Exemplary Spacer sequences*

Human β-actin 3′ UTR
taaACCGGACTGTTACCAACACCCACACCCCTGTGATGAAACAAAACCCATAAATGC (SEQ ID NO: 396)
Chicken β-actin 3′ UTR
AAGTTCTACAATGCATCTGGGACTTTGATTGTACATTGT (SEQ ID NO: 397)
Internal Deletion from Human β-actin 3′ UTR
taaACCGGACTGTaaaaaAACCCATAAATGC (SEQ ID NO: 398)
huRRBP1 5′ UTR (nucleotide 111-219), ″IRES″ D3
GGCGGCGTCAGGGTCGCAGCGTCTACAGCTGCTCGGGGGCGGTTTCTTGGCGGAGGCTTGGCCGGCTCCTCTCTCC
CGGCTCCGCGGCGGCTGCGAAGGCGGCGGCTCC (SEQ ID NO: 399)
huRRBP1 5′ UTR (nucleotide 1-69), ″IRES″ D1
CGGCGATCCCGGCGGAGGGGGCCGTTCGCCAGCTCTGAGGCAGAAAGTGCCACGACTCCACACGCGCGCCGGCGAT
CCCGGCGGAGGGGGCCGTTCGCCAGCTCTGAGGCAGAAAGTGCCACGACTCCACACGCGCGC (SEQ ID NO: 400)
huRRBP1 5′ UTR (nucleotide 70-110), ″IRES″ D2a
ACGCAGCCAGCGAGCGGCCGGAGCGGACGGCAGACGGGGCG (SEQ ID NO: 401)
huRRBP1 5′ UTR (nucleotide 220-277), ″IRES″ D2b
TGCCCTCTCGCTTTCCCTCTCGCGTCTCTGGCTGCAGGTGAAAGGAAAGCAAGCCAGG (SEQ ID NO: 402)
HCV DIV
AGACCGTGCACCATGAGCACGAATCCA (SEQ ID NO: 403)
22-mer structRNA
CACCTATATAGTTATATAATAA (SEQ ID NO: 404)
IgM1-2 pre-mRNA
caagTggacagcaaTTcacacTgTcTcTgg (SEQ ID NO: 405)
IgM-MS2 substrate
caagTggacagcaaTTcacacTgTcTcTgTcaccTgcagg (SEQ ID NO: 406)
dsx-MS2 substrate
TgTaTaaTaaaaTgccgaaTgTcTcTcgaTcTgaTcaaaccag (SEQ ID NO: 407)
longARF1
gagtgccagaagctgcctcTTcggaggcagtttctggtactc (SEQ ID NO: 408)
shortARF1
ggcagaagcTgccTcTTcggaggcagTTTcTgcc (SEQ ID NO: 409)
Scp160 binding motif
TGAAAAATTTTaaaaaGCGATGAGATaaaaaGTTAATTTG (SEQ ID NO: 410)
Mpt5 5′ UTR.1
CTCCTGTTTCATTATTTGCGTGAGGCATTCTTGTATCTCTTGCAAAGTTTTCTTTTTTCTTTCACTTTCGCACTG
(SEQ ID NO: 411)
Mpt5 5′ UTR.2
AGACTCTGTGTCTGTGTGTGCATTAGTATTTCGTCTTATTCCTTTCCTTTTCTTGCTGTACCATTGTATTTATTC
(SEQ ID NO: 412)
Mpt5 5′ UTR. 3
AAGGCCAGCTCAGCCATATACTCTTACCACATACTCAAATTCTGCTGCTCTGCTCTGCTTTTGCGTTGCTGAGTT
(SEQ ID NO: 413)
Mpt5 5′ UTR. 4
TCTTGTTTGTCTTCTAAACACTACTATAAAGCGTCTGCGTTTTTCTCTTCGTGTGTATTTCTCTTTTTTTTCGCA
(SEQ ID NO: 414)
Whi3 binding motif
GCATaaaaaGCATaaaaaGCAT (SEQ ID NO: 415)
ASH1 E1
AAGACTATGTTAAAATACGCGAAGAAGTGGCTCATTTCAAGCCATTAAGTATACCCAACTTAACTAATAATCAAAA
TAA (SEQ ID NO: 416)
ASH1 E3
ATGGATAACTGAATCTCTTTCAACTAATAAGAGACATTATCACGAAACAAT (SEQ ID NO: 299)
Vg1.VgLE.1
TATTTCTACTTTATTTCTACACTGTTA (SEQ ID NO: 300)
Vg1.VgLE.2
TTTGCCTTGACTGTTAGCTGTTACCTGTTA (SEQ ID NO: 301)
Vg1.VgLE.3
CTATTTCACTAAAATTAGATGAGGTGCTTGCACTATTCACTGCACAGAG (SEQ ID NO: 302)
PTB.1
CTCCCTCTCTCCCTCTCTTCCTTCCTCTCTCTCTTTCTCT (SEQ ID NO: 303)
PTB.2
CTCCCTCTCTCCCTCTCTTC (SEQ ID NO: 304)
PTB.3
TTCTCCTCCTTTCTTTCCTC (SEQ ID NO: 305)
PCBP.1
CCTTACCCTTTCCCCTTCCTCCAATCCCTTTCCCCTTCCA (SEQ ID NO: 306)
PCBP.2
CCTTACCCTTTCCCCTTCCT (SEQ ID NO: 307)
PCBP.3
CCAATCCCTTTCCCCTTCCA (SEQ ID NO: 308)
ALPP 3′ UTR pA signal
CAAAAATAAACAAATAAATTTTAAAAATAAATAAATAATAAAAGGAAGTGTTAGACAATGTAA (SEQ ID NO: 309)
CALR 3′ UTR pA signal
GAGAATGTAAGAACTACAAACAAAATTTCTATTAAATTAAATTTTGTGTCTC (SEQ ID NO: 310)
Rand19/35.1
ACTCATGATCAGTACAGAA (SEQ ID NO: 311)
Rand32/30.1
AGCTGACATACATATCATAACTCTACTATAGA (SEQ ID NO: 312)
Rand50/30.1
GTAAGCCTCATTTAATTACACTGTTGTAATCGTTATTATACTTTTAAGTA (SEQ ID NO: 313)
Rand50/70.1
GGTGCGGCCCGAAGCGAGTATCACCTCACGATCCAAACGACCTCTAAGAC (SEQ ID NO: 314)
pAC, 20 nt
ACCAAACAACAACACAAAAA (SEQ ID NO: 315)
pAC, 25 nt
CAACAAAAAACAAACAAAAAAAACA (SEQ ID NO: 316)
PAC, 40 nt
ACAACAACAAACAACAAAACAACAAACAACACAAAACCAA (SEQ ID NO: 41)
pAC18_1
AAAAACAAAAAACAAAA (SEQ ID NO: 317)
pAC18
AAAAACAAAAAACAAAAACCACACAAATGGTCGCCGA (SEQ ID NO: 42)
*Because the sequences are RNA sequences, it is understood that T′s represent uracil in the sequences of this table.

### MicroRNA Binding Sites

In some embodiments, the UTR of the circRNA constructs described herein comprises one or more microRNA (miRNA) binding sites. In some embodiments, the circRNA is engineered to minimize off-target expression, e.g., through the use of miRNA binding sites (engineered within spacers in the 5′ and/or 3′ UTR of the circRNA). For example, miR-122 is highly expressed in the liver, and is the most abundant miRNA in the adult liver. The use of miRNA binding sites, such as miR-122 binding sites, in the circRNA (encoding for, e.g., the CAR protein) decreases CAR expression in the liver (CAR expression in the liver is an undesirable off-target effect). The miRNA binding site in the circRNA binds to the miRNA in the liver, which leads to translation inhibition, thus reducing expression of the CAR in the liver.

In some embodiments, the number of miRNA binding sites in the circular polynucleotide is variable and relates to the length of the circular polynucleotide. As non-limiting examples, the circular polynucleotide comprises 1, 2, 3, 4, 5, 6, 7, 8, 9, 10 or more miRNA binding sites. In some other embodiments, the multiple miRNA binding sites has the same nucleic acid sequences and bind to the same miRNA. In other embodiments, the miRNA binding sites have different nucleic acid sequences and bind to different miRNAs, such as 2, 3, 4, 5, or more different miRNAs.

In some embodiments, the one or more microRNA binding sites is selected from any one of SEQ ID NOs: 27-40.

In some embodiments, the UTR comprises one microRNA binding site. In some embodiments, the 5′ UTR comprises one microRNA binding site. In preferred embodiments, the 3′ UTR comprises one microRNA binding site. In some embodiments, the circRNA comprises a single microRNA binding site. As used herein, a “single microRNA binding site” means only one microRNA binding site. In some embodiments, the one microRNA binding site is selected from any one of SEQ ID NOs: 27-40. In some embodiments, the one microRNA binding site is a miR-122 binding site. In some embodiments, the circRNA comprises a single miR-122 binding site.

In some embodiments, the single microRNA binding site is SEQ ID NO: 40.

In some embodiments, the single microRNA binding site is SEQ ID NO: 356. In the context of RNA, it is understood that SEQ ID NO: 356 is equivalent to SEQ ID NO: 40.

In some embodiments, the UTR comprises two microRNA binding sites. In some embodiments, the 5′ UTR comprises two microRNA binding sites. In some embodiments, the 3′ UTR comprises two microRNA binding sites. In some embodiments, the 5′ UTR comprises one microRNA binding site and the 3′ UTR comprises one microRNA binding site. In some embodiments, each of the two microRNA binding sites are selected from any one of SEQ ID NOs: 27-40.

In some embodiments, the UTR comprises three microRNA binding sites. In some embodiments, the 5′ UTR comprises three microRNA binding sites. In some embodiments, the 3′ UTR comprises three microRNA binding sites. In some embodiments, the 5′ UTR comprises two microRNA binding sites and the 3′ UTR comprises one microRNA binding site. In some embodiments, the 5′ UTR comprises one microRNA binding site and the 3′ UTR comprises two microRNA binding sites. In some embodiments, each of the three microRNA binding sites are selected from any one of SEQ ID NOs: 27-40.

In some embodiments, the circRNA further comprises one or more miR-122 binding sites in the 5′ UTR, the 3′ UTR, or both. In some embodiments, the microRNA binding site is a miR-122 site. In some embodiments, the miR-122 binding site is in the 3′ UTR. In some embodiments, the circRNAs described herein comprises one or more exemplary microRNA binding sites, or variants thereof, selected from Table 3.

TABLE 3
Exemplary microRNA and microRNA Binding Site Sequences*

| microRNA | | Reverse Complementary Sequence |
|---|---|---|
| (miRNA) | microRNA (miRNA) Sequence | (miRNA binding site sequence) |
| miR-3150b-3p | TGAGGAGATCGTCGAGGTTGG (SEQ ID | CCAACCTCGACGATCTCCTCA (SEQ ID |
| | NO: 13) | NO: 27) |
| miR-5196-3p | TCATCCTCGTCTCCCTCCCAG (SEQ ID | CTGGGAGGGAGACGAGGATGA (SEQ ID |
| | NO: 14) | NO: 28) |
| miR-577-5p | GTAGATAAAATATTGGTACCTG (SEQ | CAGGTACCAATATTTTATCTAC (SEQ |
| | ID NO: 15) | ID NO: 29) |
| miR-486-3p | CGGGGCAGCTCAGTACAGGAT (SEQ ID | ATCCTGTACTGAGCTGCCCCG (SEQ ID |
| | NO: 16) | NO: 30) |
| miR-577 | TAGATAAAATATTGGTACCTG (SEQ ID | CAGGTACCAATATTTTATCTA (SEQ ID |
| | NO: 17) | NO: 31) |
| miR-4424 | AGAGTTAACTCAAAATGGACTA (SEQ | TAGTCCATTTTGAGTTAACTCT (SEQ |
| | ID NO: 18) | ID NO: 32) |
| miR-10398-3p | GCCCGGAGAGCTGGGAGCCAG (SEQ ID | CTGGCTCCCAGCTCTCCGGGC (SEQ ID |
| | NO: 19) | NO: 33) |
| miR-30a-5p | TGTAAACATCCTCGACTGGAAG (SEQ | CTTCCAGTCGAGGATGTTTACA (SEQ |
| | ID NO: 20) | ID NO: 34) |
| miR-4464-5p | AAGGTTTGGATAGATGCAATA (SEQ ID | TATTGCATCTATCCAAACCTT (SEQ ID |
| | NO: 21) | NO: 35) |
| miR-151a-3p | CTAGACTGAAGCTCCTTGAGG (SEQ ID | CCTCAAGGAGCTTCAGTCTAG (SEQ ID |
| | NO: 22) | NO: 36) |
| miR-142-3p | CATAAAGTAGAAAGCACTACT (SEQ ID | AGTAGTGCTTTCTACTTTATG (SEQ ID |
| | NO: 23) | NO: 37) |
| miR-125a-3p | ACAGGTGAGGTTCTTGGGAGCC (SEQ | GGCTCCCAAGAACCTCACCTGT (SEQ |
| | ID NO: 24) | ID NO: 38) |
| miR-31-5p | AGGCAAGATGCTGGCATAGCT (SEQ ID | AGCTATGCCAGCATCTTGCCT (SEQ ID |
| | NO: 25) | NO: 39) |
| miR-122-5p | TGGAGTGTGACAATGGTGTTTG (SEQ | CAAACACCATTGTCACACTCCA (SEQ |
| | ID NO: 26) | ID NO: 40) |

*Because the sequences are RNA sequences, it is understood that T's represent uracil in the sequences of this table.

### Codon-Optimized Coding Sequences

The present invention provides, among other things, circRNA molecules comprising coding sequences encoding for anti-CD19 CAR. The coding sequence, also known as open reading frame (ORF) sequence, is defined herein as the region that encodes the protein of interest.

In some embodiments, the coding sequence includes a region to initiate translation. In some embodiments, the region includes any translation initiation sequence or signal including a start codon. As a non-limiting example, the coding sequence includes one or more start codon(s). In some embodiments, as related to a DNA sequence, the start codon is “ATG,” “ACG,” “AGG,” “ATA,” “ATT,” “CTG,” “GTG,” or “TTG.” In some embodiments, as related to an RNA sequence, the start codon is “AUG,” “AUA,” “AUU,” “CUG,” “GUG,” or “UUG.”

In some embodiments, the coding sequence further includes a region to stop translation. This region may include any translation termination sequence or signal including a stop codon. As a non-limiting example, the region includes one or more stop codon(s). In some embodiments, as related to a DNA sequence, the stop codon is “TGA,” “TAA,” “TGA,” or “TAG.” In some embodiments, as related to an RNA sequence, the stop codon is “UGA,” “UAA,” “UGA” or “UAG.”

In some embodiments, the circRNA molecule of the present disclosure comprises a coding sequence. In some embodiments, the circRNA molecule of the present disclosure comprises a codon-optimized coding sequence. In some embodiments, the CDS comprises a nucleic acid sequence or variants thereof selected from Table 4, which are exemplary codon-optimized codon sequences comprising anti-CD19 antigen-binding domain.

In some aspects, provided herein is a circRNA comprising a codon-optimized sequence having at least 75% identity to SEQ ID NOS: 125-168 or 266. In some aspects, provided herein is a circRNA comprising a codon-optimized sequence having at least 80% identity to SEQ ID NOS: 125-168 or 266. In some aspects, provided herein is a circRNA comprising a codon-optimized sequence having at least 85% identity to SEQ ID NOS: 125-168 or 266. In some aspects, provided herein is a circRNA comprising a codon-optimized sequence having at least 90% identity to SEQ ID NOS: 125-168 or 266. In some aspects, provided herein is a circRNA comprising a codon-optimized sequence having at least 95% identity to SEQ ID NOS: 125-168 or 266. In some embodiments, the circRNA comprises a codon-optimized sequence having 100% identity to SEQ ID NOS: 125-168 or 266.

TABLE 4
Exemplary Codon-Optimized Coding Sequence* Comprising Anti-CD19
Antigen-Binding Domain

ATGGCTCTGCCTGTGACAGCTCTGCTGCTGCCTCTGGCTCTGCTTCTGCATGCCGCCAGACCTGACATCCAGATGA
CCCAGACAACCAGCAGCCTGTCTGCCAGCCTGGGCGACAGAGTGACCATCAGCTGTAGAGCCAGCCAGGACATCAG
CAAGTACCTGAACTGGTATCAGCAGAAACCCGACGGCACCGTGAAGCTGCTGATCTACCACACCAGCAGACTGCAC
AGCGGCGTGCCATCTAGATTTTCTGGCAGCGGCTCTGGCACCGACTACAGCCTGACAATCAGCAACCTGGAACAAG
AGGATATCGCTACCTACTTCTGCCAGCAAGGCAACACCCTGCCTTACACATTCGGCGGAGGCACCAAGCTGGAAAT
CACAGGTGGCGGAGGATCTGGCGGAGGTGGAAGCGGAGGCGGTGGATCTGAAGTGAAACTGCAAGAGTCTGGCCCT
GGCCTGGTGGCTCCATCTCAGTCTCTGAGCGTGACCTGTACCGTCAGCGGAGTGTCTCTGCCTGATTACGGCGTGT
CCTGGATCAGACAGCCTCCTAGAAAAGGCCTGGAATGGCTGGGCGTGATCTGGGGCAGCGAGACAACCTACTACAA
CAGCGCCCTGAAGTCCAGGCTGACCATCATCAAGGACAACTCCAAGAGCCAGGTGTTCCTGAAGATGAACAGCCTG
CAGACCGACGACACCGCCATCTACTACTGCGCCAAGCACTACTACTACGGCGGCAGCTACGCCATGGATTACTGGG
GCCAGGGAACCAGCGTGACCGTGTCTAGCACAACAACCCCTGCTCCTAGACCTCCTACACCAGCTCCTACAATCGC
CAGCCAGCCTCTGTCTCTGAGGCCAGAGGCTTGTAGACCTGCTGCTGGCGGAGCTGTGCACACAAGAGGCCTGGAT
TTCGCCTGCGACATCTACATCTGGGCCCCTCTGGCTGGAACATGTGGCGTGCTGCTGCTGAGCCTGGTCATCACCC
TGTATTGCAAGAGAGGCCGGAAGAAGCTGCTCTACATCTTCAAGCAGCCCTTCATGAGGCCCGTGCAGACCACACA
AGAAGAGGACGGCTGCTCCTGCAGATTCCCCGAGGAAGAGGAAGGCGGCTGCGAGCTGAGAGTGAAGTTCAGCAGA
AGCGCTGACGCCCCTGCTTACCAGCAGGGACAGAACCAGCTGTACAACGAGCTGAACCTGGGGAGAAGAGAAGAGT
ACGACGTGCTGGACAAGCGGAGAGGCAGAGATCCTGAGATGGGCGGCAAGCCCAGAAGAAAGAACCCTCAAGAGGG
CCTGTATAATGAGCTGCAGAAAGACAAGATGGCCGAGGCCTACAGCGAGATCGGAATGAAGGGCGAGCGCAGAAGA
GGCAAGGGACACGATGGACTGTACCAGGGCCTGAGCACAGCCACCAAGGACACATATGACGCCCTGCACATGCAGG
CCCTGCCTCCAAGA (SEQ ID NO: 125)
ATGGCACTACCCGTTACAGCATTACTATTACCTCTCGCCCTCCTCCTCCACGCCGCCAGACCAGATATCCAGATGA
CACAGACCACCTCTAGCCTCAGCGCCAGCCTCGGCGATAGAGTGACAATCTCTTGCAGAGCCTCCCAGGACATCAG
CAAGTACCTCAATTGGTACCAGCAGAAGCCCGACGGCACCGTGAAGCTCCTGATCTACCACACCAGCAGGCTGCAC
AGCGGCGTGCCATCTAGATTCTCTGGATCTGGATCTGGCACCGATTACAGCCTGACCATCAGCAATCTGGAGCAGG
AGGACATCGCCACCTACTTCTGCCAGCAGGGGAATACCCTGCCCTACACCTTTGGCGGCGGCACAAAGCTGGAGAT
TACAGGTGGTGGTGGTTCTGGGGGAGGAGGGTCTGGTGGTGGTGGTAGTGAAGTGAAACTGCAGGAATCTGGACCT
GGCCTGGTGGCCCCTTCTCAGTCGCTGAGCGTGACATGCACCGTGTCTGGCGTGAGCCTGCCGGATTACGGCGTGA
GCTGGATTCGGCAGCCTCCTCGGAAGGGCCTGGAATGGCTGGGCGTGATCTGGGGCTCCGAGACCACCTACTACAA
CAGCGCCCTGAAGTCCAGGCTGACCATCATCAAGGACAACAGCAAGTCCCAGGTGTTCCTGAAGATGAACAGCCTG
CAGACCGATGACACGGCCATCTACTACTGCGCCAAGCACTACTACTACGGAGGCTCCTACGCCATGGATTACTGGG
GCCAGGGAACCTCCGTGACAGTGTCTTCTACAACAACACCAGCTCCTAGGCCTCCTACACCTGCTCCTACAATTGC
TTCTCAGCCTCTGTCTCTGAGACCAGAAGCTTGTAGACCAGCTGCTGGAGGAGCTGTGCATACAAGAGGCCTGGAT
TTCGCCTGTGACATTTACATCTGGGCTCCACTGGCCGGCACCTGTGGCGTGCTGCTGCTGAGCCTGGTGATCACCC
TGTACTGCAAGAGGGGCAGAAAGAAGCTGCTGTACATCTTCAAGCAGCCTTTCATGCGCCCCGTGCAGACAACACA
GGAGGAAGATGGCTGCAGCTGTAGATTTCCTGAAGAAGAGGAGGGCGGCTGCGAGCTGAGAGTGAAATTTTCTAGA
AGCGCCGACGCCCCTGCCTACCAGCAGGGCCAGAATCAGTTATATAACGAACTGAATCTGGGCCGGCGGGAGGAGT
ACGACGTGCTGGATAAGAGAAGAGGCAGAGATCCTGAAATGGGAGGAAAACCTAGAAGAAAGAATCCGCAGGAGGG
CTTATATAATGAACTGCAGAAGGACAAGATGGCGGAGGCCTACAGCGAGATCGGCATGAAGGGCGAGAGAAGAAGA
GGCAAGGGCCACGATGGCCTGTACCAGGGCCTGAGCACGGCCACCAAGGATACCTACGATGCCCTGCACATGCAGG
CCCTGCCTCCTAGA (SEQ ID NO: 126)
ATGGCATTACCAGTTACAGCCCTCCTTCTTCCTCTCGCCCTCTTGCTCCACGCCGCCAGGCCAGATATCCAGATGA
CACAGACCACCAGCAGCCTCAGCGCCAGCCTCGGCGATAGAGTGACAATCTCTTGCAGAGCCTCCCAGGACATCAG
CAAGTACCTCAATTGGTACCAGCAGAAGCCCGACGGCACCGTGAAGTTGCTGATCTACCACACCAGCAGACTGCAC
AGCGGAGTGCCTTCTAGATTCTCTGGAAGCGGCTCCGGCACAGACTACTCGCTGACCATCAGCAACCTGGAGCAGG
AGGACATCGCCACCTACTTCTGCCAGCAGGGCAATACCCTGCCCTACACCTTCGGCGGCGGCACAAAACTGGAGAT
TACAGGTGGTGGTGGTAGTGGTGGTGGGGGAAGTGGTGGTGGTGGTTCTGAAGTGAAACTGCAGGAATCAGGGCCT
GGCCTGGTGGCCCCTTCTCAGTCGCTGAGCGTGACCTGTACCGTGTCAGGCGTGAGCCTGCCAGATTACGGAGTGA
GCTGGATTAGACAGCCCCCTCGGAAGGGCCTGGAATGGCTGGGCGTGATCTGGGGCAGCGAGACCACCTACTACAA
CAGCGCCCTGAAGTCCAGGCTGACCATCATCAAGGACAACAGCAAGTCCCAGGTGTTCCTGAAGATGAACAGCCTG
CAGACCGACGACACCGCCATCTACTACTGCGCCAAGCACTACTACTACGGCGGCAGCTACGCCATGGATTACTGGG
GCCAGGGCACCTCTGTGACGGTGTCTTCTACAACAACACCTGCTCCTAGGCCTCCTACACCAGCTCCTACAATTGC
TTCTCAGCCGCTGAGTCTGCGGCCAGAAGCTTGTAGGCCTGCTGCAGGAGGAGCTGTGCATACAAGAGGACTGGAC
TTTGCCTGTGATATCTACATCTGGGCCCCTCTGGCCGGCACCTGCGGCGTGCTGCTGCTGAGCCTGGTGATCACCC
TGTACTGCAAGCGCGGCCGGAAGAAGCTGCTGTACATCTTCAAGCAGCCCTTCATGAGGCCCGTGCAGACAACACA
GGAGGAGGATGGCTGCTCCTGTAGATTTCCTGAAGAGGAGGAGGGCGGCTGCGAGCTGAGAGTGAAATTTTCTAGA
AGCGCCGATGCCCCTGCCTACCAGCAGGGCCAGAACCAGTTATATAACGAACTGAACCTGGGCAGAAGAGAGGAGT
ACGACGTGCTGGATAAGAGAAGAGGCAGAGATCCAGAAATGGGAGGAAAGCCTAGAAGAAAGAATCCGCAGGAGGG
CCTATATAATGAACTGCAGAAGGACAAGATGGCCGAGGCCTACAGCGAGATCGGCATGAAGGGCGAGAGAAGAAGA
GGCAAGGGCCACGATGGACTGTATCAGGGGCTGAGCACCGCCACCAAGGATACCTACGATGCCCTGCACATGCAGG
CGCTGCCTCCTAGA (SEQ ID NO: 127)
ATGGCATTACCAGTTACAGCCCTCCTCCTCCCTCTCGCCCTCCTTCTCCACGCCGCCAGACCAGATATCCAGATGA
CCCAGACCACCTCTAGCCTCAGCGCCAGCCTCGGCGATAGAGTGACAATCTCTTGCAGAGCCTCCCAGGACATCAG
CAAGTACCTCAATTGGTACCAGCAGAAGCCCGACGGCACCGTGAAGCTCCTGATCTACCACACCAGCAGGCTGCAC
TCTGGCGTGCCATCTAGATTTTCTGGATCTGGCAGCGGCACCGACTACAGCCTGACCATCAGCAACCTGGAGCAGG
AGGACATCGCCACCTACTTCTGCCAGCAGGGGAATACCCTGCCCTACACCTTTGGCGGCGGCACAAAGCTGGAGAT
TACAGGTGGTGGTGGTTCTGGTGGTGGTGGTAGTGGTGGTGGGGGATCTGAAGTGAAACTGCAGGAATCTGGACCT
GGCCTGGTGGCCCCTTCTCAGTCTCTGAGCGTGACATGCACCGTGTCCGGCGTGAGCCTGCCGGATTACGGCGTGA
GCTGGATTCGGCAGCCTCCTAGAAAGGGCCTGGAATGGCTGGGCGTGATCTGGGGCAGCGAGACCACCTACTACAA
CAGCGCGCTGAAGTCCAGGCTGACCATCATCAAGGACAACAGCAAGTCCCAGGTGTTCCTGAAGATGAACAGCCTG
CAGACCGATGACACCGCCATCTACTACTGTGCGAAGCACTACTACTACGGCGGCTCCTACGCCATGGATTACTGGG
GCCAGGGCACCAGCGTGACGGTGTCTTCTACAACAACACCTGCTCCTAGACCTCCTACACCAGCTCCTACAATTGC
TTCTCAGCCTCTGTCGCTGAGACCTGAAGCATGTAGACCTGCTGCTGGAGGAGCTGTGCATACAAGAGGACTGGAT
TTTGCCTGCGACATCTACATCTGGGCCCCGCTGGCCGGCACCTGCGGCGTGCTGCTGCTGAGCCTGGTGATCACCC
TGTACTGCAAGAGGGGCAGGAAGAAGCTGCTGTACATCTTCAAGCAGCCCTTCATGAGGCCTGTGCAGACCACCCA
GGAGGAGGATGGCTGCAGCTGCAGATTTCCTGAAGAAGAGGAGGGAGGCTGCGAGCTGAGAGTGAAATTTTCCAGG
AGCGCCGACGCTCCTGCCTACCAGCAGGGCCAGAATCAGTTATATAATGAACTGAACCTGGGCCGGCGGGAGGAGT
ACGACGTGCTGGATAAGAGAAGAGGCAGAGATCCTGAAATGGGAGGAAAGCCTAGAAGAAAGAATCCGCAGGAGGG
CCTATATAACGAACTGCAGAAGGACAAGATGGCCGAGGCCTATTCCGAGATCGGAATGAAGGGCGAGAGAAGAAGA
GGCAAGGGCCACGATGGCCTGTACCAGGGCCTGAGCACGGCCACCAAGGATACCTACGACGCCCTGCACATGCAGG
CCCTGCCTCCTAGA (SEQ ID NO: 128)
ATGGCCCTACCCGTTACAGCTCTACTGCTGCCGCTGGCACTCTTGCTGCACGCAGCCAGACCTGACATTCAGATGA
CGCAGACTACGTCGTCCCTGTCAGCGTCCCTTGGGGATCGGGTTACTATAAGTTGCCGCGCCAGCCAGGATATAAG
TAAATACCTCAATTGGTATCAGCAAAAGCCGGATGGTACCGTGAAGCTCCTCATCTACCATACGTCCCGGCTCCAC
TCTGGTGTGCCGAGCAGGTTCTCCGGGAGTGGGAGTGGGACAGACTACAGCCTCACGATCAGCAATCTAGAGCAGG
AGGACATCGCTACTTACTTTTGCCAGCAGGGGAATACCCTTCCCTACACTTTCGGTGGCGGCACGAAGTTGGAGAT
AACTGGAGGCGGCGGGTCTGGCGGCGGTGGGTCAGGCGGTGGGGGCTCGGAGGTGAAGCTGCAAGAAAGCGGTCCA
GGGCTGGTGGCCCCTTCACAGTCCCTTAGTGTGACCTGCACTGTCAGCGGAGTCTCACTGCCCGATTACGGGGTGA
GCTGGATCAGGCAACCGCCGAGGAAGGGGCTGGAATGGTTAGGTGTCATCTGGGGGTCAGAGACTACTTACTATAA
CAGCGCTTTGAAGTCTAGACTGACTATCATTAAGGATAACTCCAAATCCCAAGTCTTCCTCAAGATGAACAGTTTA
CAGACAGATGACACAGCCATCTACTACTGTGCCAAGCACTACTACTACGGAGGCTCATATGCGATGGACTATTGGG
GCCAAGGTACGTCGGTTACAGTAAGCTCGACGACCACTCCCGCACCTCGGCCTCCCACCCCTGCTCCTACCATCGC
CAGTCAACCCCTGTCCCTGCGGCCTGAGGCCTGTCGTCCCGCGGCCGGGGGTGCGGTCCATACGCGCGGACTTGAC
TTTGCGTGTGATATCTACATTTGGGCCCCCTTAGCTGGCACATGTGGTGTTCTCCTGCTCTCCCTGGTCATCACCT
TGTACTGCAAGCGTGGTCGCAAGAAGTTGCTATATATATTTAAGCAGCCCTTCATGCGGCCAGTCCAGACTACTCA
GGAAGAAGATGGCTGTAGTTGCCGGTTCCCCGAGGAAGAGGAAGGCGGCTGCGAGCTGCGGGTTAAGTTCTCTCGC
AGTGCCGATGCCCCTGCGTATCAGCAGGGGCAGAATCAACTGTATAACGAGTTGAATCTTGGCCGTCGGGAAGAGT
ACGATGTTCTCGATAAACGGCGGGGTCGAGATCCCGAGATGGGAGGGAAGCCCCGGAGAAAGAACCCACAAGAGGG
GCTTTATAACGAGCTGCAGAAAGACAAGATGGCCGAGGCCTACTCGGAGATCGGGATGAAGGGAGAACGGCGCCGG
GGTAAGGGCCACGACGGCCTTTACCAAGGGTTAAGTACCGCGACAAAAGATACTTATGACGCACTGCACATGCAGG
CGTTGCCGCCAAGG (SEQ ID NO: 129)
ATGGCCTTGCCGGTCACTGCATTGCTGCTGCCTCTCGCACTGCTGCTTCATGCCGCAAGGCCTGACATTCAGATGA
CTCAGACAACCAGCTCGCTCAGCGCATCGCTGGGGGATCGCGTGACGATCAGTTGCCGTGCATCCCAGGATATTTC
CAAGTACCTCAACTGGTACCAGCAGAAGCCAGATGGTACAGTAAAATTGCTGATATACCATACATCCAGATTACAC
TCAGGCGTACCCTCGCGCTTTAGTGGCTCCGGAAGTGGCACCGATTACTCTTTGACCATCAGTAATCTCGAGCAAG
AAGACATTGCAACGTACTTCTGCCAACAGGGGAACACTCTCCCATACACCTTTGGGGGAGGTACTAAGCTCGAGAT
TACGGGTGGCGGGGGGTCGGGCGGTGGCGGCTCTGGAGGCGGAGGGAGCGAGGTTAAACTGCAGGAGTCAGGCCCT
GGGCTTGTAGCTCCCAGTCAGTCACTGTCTGTGACTTGTACTGTGTCAGGTGTTTCCTTGCCAGACTATGGGGTAT
CCTGGATCCGCCAGCCACCCCGTAAAGGTCTGGAGTGGCTCGGGGTCATATGGGGCTCCGAAACTACTTATTACAA
CTCGGCATTGAAGAGCAGGCTGACAATTATTAAAGATAATAGTAAGTCGCAGGTGTTCCTTAAAATGAACTCCCTG
CAAACTGATGATACGGCCATATACTATTGTGCCAAACACTATTATTACGGCGGCAGCTATGCTATGGACTATTGGG
GTCAGGGCACATCTGTTACCGTTAGTTCGACGACAACGCCCGCACCGCGCCCTCCAACCCCAGCTCCGACGATTGC
ATCGCAGCCCCTCTCACTGCGCCCCGAGGCCTGTCGGCCAGCCGCAGGTGGCGCTGTGCATACACGGGGGCTGGAC
TTCGCATGCGATATTTATATCTGGGCTCCACTGGCCGGGACGTGCGGAGTTCTTCTCCTGTCACTGGTGATCACCC
TGTATTGTAAGCGCGGGCGGAAGAAGCTGCTATATATTTTTAAGCAGCCCTTTATGAGGCCTGTGCAGACGACACA
GGAAGAAGACGGGTGCAGTTGCCGATTCCCTGAGGAAGAGGAGGGGGGCTGCGAGCTGCGCGTGAAATTTTCGCGC
TCCGCTGACGCACCTGCGTACCAGCAAGGCCAGAATCAACTCTACAATGAGTTGAATTTGGGGAGACGCGAAGAGT
ACGACGTCCTCGACAAGCGACGAGGGAGGGATCCTGAAATGGGAGGAAAGCCTCGCAGAAAGAACCCTCAGGAGGG
GTTGTACAATGAGTTACAGAAGGACAAGATGGCGGAAGCCTACTCCGAGATAGGGATGAAGGGCGAGCGGAGGCGG
GGGAAGGGCCACGACGGGCTTTATCAAGGTCTGTCCACAGCCACCAAGGATACCTATGACGCTCTGCATATGCAGG
CCCTGCCCCCACGC (SEQ ID NO: 130)
ATGGCCCTTCCAGTGACAGCATTGTTGCTTCCTTTGGCACTGCTGTTACATGCCGCCCGCCCCGACATTCAGATGA
CACAGACAACTTCCTCACTGAGTGCCAGTCTCGGAGACAGAGTGACCATCAGCTGTCGGGCCTCCCAAGATATCAG
TAAATACCTTAACTGGTACCAGCAGAAGCCCGATGGGACAGTTAAACTTCTCATCTATCATACCAGCAGGCTGCAC
AGCGGCGTTCCTAGCAGATTTAGTGGTAGCGGGAGCGGCACTGATTACAGCCTGACAATTAGCAATCTCGAGCAGG
AGGATATCGCTACCTACTTTTGCCAGCAGGGTAACACCCTGCCCTACACTTTCGGGGGGGGAACTAAACTGGAGAT
AACAGGGGGTGGCGGGAGCGGGGGAGGTGGCAGTGGGGGGGGCGGGAGTGAAGTGAAACTCCAGGAGAGCGGGCCA
GGTCTCGTGGCCCCCTCTCAATCTTTGTCAGTGACTTGCACAGTCTCTGGCGTTTCCCTGCCCGACTATGGCGTCT
CATGGATTCGACAGCCCCCTCGAAAGGGGTTAGAGTGGCTCGGAGTGATATGGGGCTCCGAAACCACCTATTACAA
TTCCGCTTTAAAGAGCAGATTGACAATCATCAAGGATAATTCCAAGTCTCAGGTGTTTTTGAAAATGAACTCCCTT
CAGACTGATGACACTGCAATCTACTACTGTGCCAAACATTACTATTACGGGGGGAGTTACGCCATGGACTACTGGG
GGCAGGGCACAAGTGTGACTGTCTCCTCCACCACCACTCCTGCTCCCCGACCTCCTACACCTGCTCCAACTATCGC
CTCTCAGCCACTGAGCCTGCGACCCGAGGCTTGCAGGCCTGCAGCTGGAGGCGCTGTCCATACCAGAGGTCTGGAC
TTCGCCTGCGATATCTACATCTGGGCCCCACTAGCCGGTACCTGCGGCGTCCTCTTATTGAGCCTCGTGATCACTC
TTTACTGCAAGAGAGGCCGCAAGAAGCTGTTGTACATATTCAAACAGCCTTTTATGCGGCCTGTGCAGACTACACA
GGAGGAAGACGGGTGCTCATGTAGATTCCCTGAGGAAGAGGAGGGTGGATGCGAGCTTCGGGTGAAGTTCAGCCGA
AGCGCTGACGCCCCCGCATACCAACAGGGCCAGAACCAACTCTACAATGAGTTGAATTTGGGCCGCAGGGAAGAAT
ACGACGTGCTGGATAAACGAAGGGGCCGAGACCCCGAAATGGGGGGCAAGCCTCGGCGGAAGAATCCCCAGGAAGG
CTTGTACAATGAGTTGCAGAAAGATAAAATGGCCGAGGCCTACTCCGAGATTGGTATGAAGGGCGAAAGAAGGCGG
GGAAAAGGGCACGACGGGTTGTATCAGGGGCTGAGTACCGCAACAAAGGACACCTATGATGCCCTACATATGCAGG
CTCTACCTCCCAGA (SEQ ID NO: 131)
ATGGCTCTTCCCGTGACAGCCTTGCTGCTCCCATTGGCTCTCCTACTGCACGCAGCACGACCCGATATTCAGATGA
CCCAGACTACCTCTTCCCTCAGCGCCTCCCTCGGGGACCGAGTGACTATCTCCTGTCGGGCATCCCAGGATATTAG
TAAGTACCTCAATTGGTACCAGCAAAAGCCCGACGGGACAGTCAAACTATTAATATACCACACCTCTAGACTACAT
AGTGGTGTTCCTTCTCGGTTCTCCGGGAGCGGCTCTGGGACTGACTACAGTCTCACCATAAGCAACCTGGAGCAGG
AGGACATAGCCACATACTTCTGTCAGCAGGGCAACACCCTGCCCTATACCTTCGGAGGGGGCACCAAACTAGAGAT
TACTGGCGGAGGGGGTTCAGGAGGAGGCGGGAGCGGGGGCGGTGGAAGTGAGGTGAAGTTACAGGAGTCCGGGCCT
GGGCTCGTCGCCCCTTCCCAGAGCCTGTCAGTTACCTGTACTGTGAGCGGAGTCTCATTACCCGACTACGGAGTCA
GCTGGATCCGCCAGCCCCCCAGGAAGGGACTGGAGTGGCTGGGCGTGATCTGGGGTTCTGAGACCACATATTATAA
TTCTGCTCTAAAATCTAGGCTGACAATCATCAAAGACAACAGCAAGAGCCAGGTGTTTCTGAAGATGAACTCATTA
CAAACAGATGACACTGCTATCTACTACTGCGCAAAGCACTACTATTATGGTGGGAGCTATGCCATGGATTATTGGG
GGCAGGGGACCAGCGTCACTGTCTCCAGTACAACAACCCCCGCTCCACGACCTCCAACCCCAGCACCCACTATTGC
TTCACAGCCCCTGAGTTTACGGCCCGAGGCATGTCGCCCTGCCGCTGGAGGGGCTGTTCATACCAGAGGACTGGAC
TTCGCCTGTGATATCTACATTTGGGCCCCACTGGCCGGAACATGTGGCGTTCTCCTGCTCTCCCTCGTCATAACAC
TCTATTGCAAGAGAGGCAGGAAGAAACTTCTCTATATCTTTAAACAGCCTTTTATGAGGCCTGTGCAGACTACACA
GGAAGAGGATGGATGTTCCTGCCGATTCCCAGAGGAGGAGGAGGGCGGGTGTGAGCTACGAGTGAAGTTCTCACGC
TCTGCAGATGCCCCCGCTTACCAGCAGGGGCAGAATCAGCTCTACAACGAGCTGAATCTCGGACGCCGAGAAGAAT
ACGACGTGCTGGACAAACGCAGGGGTAGAGACCCCGAGATGGGGGGAAAACCTAGGCGCAAAAACCCCCAGGAGGG
CCTGTATAACGAGCTGCAAAAGGATAAGATGGCCGAAGCTTACAGTGAGATCGGCATGAAGGGGGAGCGCCGCCGC
GGTAAAGGGCACGACGGTCTGTACCAGGGACTTAGCACAGCTACCAAGGACACCTACGATGCTCTCCACATGCAGG
CATTACCACCCAGG (SEQ ID NO: 132)
ATGGCCTTGCCCGTCACTGCCCTATTACTTCCTCTCGCCCTGTTGCTCCACGCCGCTCGCCCCGACATCCAAATGA
CACAGACCACCTCTTCCCTATCCGCATCCTTGGGAGATAGGGTTACCATCTCCTGTAGGGCATCCCAGGATATAAG
CAAATATTTGAATTGGTATCAGCAAAAACCTGATGGTACAGTGAAGCTACTCATTTATCACACATCCCGGTTGCAT
TCCGGCGTGCCCTCCCGCTTCAGCGGAAGCGGTTCCGGGACTGACTATTCACTTACTATAAGTAATCTAGAGCAAG
AGGACATTGCAACTTATTTCTGCCAGCAAGGAAACACCCTACCCTACACATTTGGAGGAGGAACAAAATTGGAAAT
CACCGGGGGCGGGGGTTCAGGTGGTGGTGGAAGCGGTGGTGGTGGATCAGAGGTTAAGTTGCAAGAGAGCGGCCCC
GGCCTTGTGGCTCCCAGTCAGAGTCTATCAGTGACCTGTACCGTGTCTGGCGTTAGTCTCCCAGACTATGGAGTTA
GCTGGATCAGACAACCACCACGCAAGGGACTGGAGTGGCTTGGAGTTATATGGGGATCTGAGACCACATATTACAA
TTCTGCCCTTAAGTCCCGACTAACAATCATTAAAGACAACTCTAAGAGCCAGGTTTTCCTCAAAATGAACTCTTTG
CAGACCGATGACACCGCTATTTACTATTGTGCCAAGCACTACTATTACGGTGGGTCATACGCTATGGATTACTGGG
GGCAGGGCACATCAGTCACTGTGAGCTCTACAACCACACCCGCTCCCAGGCCTCCAACCCCCGCCCCCACTATCGC
CTCTCAACCTCTCAGCTTAAGGCCAGAGGCTTGTCGACCTGCTGCCGGCGGAGCTGTTCATACCCGCGGTTTGGAC
TTCGCTTGCGACATATACATTTGGGCACCACTCGCCGGCACCTGCGGTGTGCTCCTCCTTTCTCTCGTGATTACCC
TGTATTGCAAGCGAGGCAGAAAAAAATTATTATATATTTTTAAACAGCCCTTCATGCGGCCTGTGCAGACTACACA
AGAAGAAGATGGGTGCAGCTGCAGGTTCCCTGAGGAGGAGGAAGGCGGCTGTGAACTTCGGGTCAAATTTTCCCGC
AGTGCCGATGCTCCTGCATACCAACAAGGTCAAAACCAGCTGTACAATGAGTTAAATCTTGGACGGAGAGAAGAGT
ATGATGTCTTGGACAAGCGGCGAGGTCGGGATCCTGAGATGGGCGGAAAGCCACGGCGCAAGAACCCACAAGAAGG
TCTCTACAACGAGCTTCAGAAAGATAAAATGGCTGAAGCTTATAGCGAAATTGGGATGAAAGGAGAAAGACGCAGG
GGAAAGGGACACGACGGACTTTATCAAGGTCTATCTACCGCTACCAAAGATACTTATGACGCCCTCCACATGCAGG
CACTTCCTCCACGC (SEQ ID NO: 133)
ATGGCCTTGCCAGTAACCGCACTTCTGCTGCCACTAGCACTGCTGCTGCATGCAGCCCGACCAGATATACAAATGA
CCCAGACAACCTCGAGTTTGAGTGCCTCCCTTGGAGATCGCGTAACTATCAGCTGTCGGGCTAGTCAGGACATTTC
AAAGTACCTCAACTGGTATCAGCAGAAACCTGATGGAACTGTGAAGCTGCTGATTTACCATACATCACGCTTACAC
TCCGGAGTGCCCTCACGTTTCTCCGGATCCGGTTCCGGAACTGATTATAGTCTTACCATTAGCAACCTTGAGCAGG
AAGACATAGCCACCTATTTCTGCCAACAAGGAAATACTTTGCCTTATACCTTTGGGGGCGGCACTAAGCTGGAAAT
CACAGGTGGCGGGGGAAGCGGGGGCGGGGGAAGTGGCGGTGGGGGGTCTGAAGTGAAGCTCCAAGAATCTGGACCA
GGTTTAGTTGCACCGAGCCAAAGCCTATCCGTGACCTGCACTGTTTCCGGGGTGTCTCTTCCAGATTACGGAGTGT
CCTGGATACGCCAGCCGCCGCGAAAAGGGCTCGAGTGGTTGGGCGTCATATGGGGCAGCGAAACGACCTATTATAA
CAGTGCCCTGAAAAGCCGGCTAACGATCATTAAAGATAACTCTAAGTCACAAGTATTCCTCAAGATGAACTCCCTT
CAGACGGATGATACGGCCATATACTACTGTGCTAAGCATTATTATTATGGCGGGTCATACGCGATGGACTACTGGG
GCCAGGGGACATCCGTCACCGTATCCTCTACGACGACACCGGCACCAAGACCTCCTACTCCTGCACCCACCATAGC
CAGCCAGCCTTTGTCACTGAGACCCGAGGCTTGTAGACCAGCTGCCGGTGGCGCAGTGCACACCCGAGGCTTGGAC
TTTGCCTGTGACATTTACATCTGGGCACCACTTGCAGGGACTTGCGGGGTACTCTTATTGAGCCTCGTAATCACAC
TGTACTGCAAGAGAGGACGCAAAAAATTGCTATATATTTTTAAACAGCCTTTTATGCGCCCTGTTCAGACTACGCA
GGAGGAAGATGGATGTTCTTGTAGGTTTCCCGAAGAAGAGGAGGGCGGGTGTGAGCTAAGAGTAAAATTCAGTAGA
TCTGCGGATGCACCGGCCTACCAGCAGGGTCAGAACCAGTTATACAACGAGCTAAACTTAGGACGCAGAGAAGAGT
ACGATGTATTAGATAAACGCCGTGGTCGCGATCCTGAAATGGGTGGTAAGCCTCGTCGGAAGAATCCTCAGGAGGG
ACTGTACAACGAACTACAGAAAGATAAGATGGCTGAGGCTTACTCCGAGATTGGAATGAAGGGAGAGCGCAGAAGG
GGAAAGGGACATGACGGCCTGTACCAAGGACTGAGCACTGCAACTAAGGATACCTACGACGCTCTGCACATGCAGG
CCCTGCCCCCAAGA (SEQ ID NO: 134)
ATGGCTCTACCAGTCACAGCACTACTCCTCCCTCTTGCCCTCCTTCTTCACGCCGCCAGGCCAGATATCCAGATGA
CACAGACCACCTCCTCCCTTTCCGCCTCTCTCGGGGACCGAGTCACTATTTCTTGTCGGGCTTCTCAGGATATCAG
TAAGTACCTTAATTGGTACCAGCAGAAGCCCGACGGGACAGTGAAGCTCCTGATCTACCACACCAGTAGGCTCCAC
TCTGGTGTCCCCTCCAGATTTAGTGGGTCAGGGAGTGGGACCGACTACTCCCTGACCATCTCCAATCTGGAGCAGG
AGGATATCGCCACCTACTTCTGTCAGCAGGGGAATACCCTGCCATATACATTTGGAGGGGGCACCAAGCTGGAGAT
TACTGGTGGTGGTGGATCAGGAGGAGGTGGAAGTGGAGGAGGTGGTTCTGAAGTGAAACTTCAGGAATCTGGACCT
GGCCTGGTGGCTCCCAGCCAGTCCCTGAGTGTGACTTGCACCGTGTCTGGCGTGTCTCTGCCAGACTACGGTGTGA
GCTGGATCCGCCAGCCACCCAGGAAAGGCCTGGAGTGGCTGGGCGTGATCTGGGGCAGTGAGACCACCTACTATAA
TAGTGCTTTGAAGTCCAGGCTGACCATCATCAAGGACAACTCCAAGAGTCAGGTGTTCTTGAAGATGAACAGCCTG
CAGACTGATGATACCGCAATCTACTATTGCGCAAAGCACTATTATTATGGGGGGTCTTACGCCATGGATTACTGGG
GGCAGGGGACTAGTGTTACTGTGAGTAGCACTACTACCCCTGCCCCTAGACCTCCAACACCAGCTCCTACCATTGC
AAGTCAGCCTCTGTCTCTGAGACCAGAGGCTTGTAGACCTGCAGCTGGTGGAGCTGTGCATACTCGGGGACTGGAT
TTTGCATGTGACATTTACATCTGGGCTCCTCTGGCCGGCACCTGCGGCGTCCTCCTCCTGAGTCTGGTGATCACTC
TGTACTGCAAGCGCGGAAGGAAGAAGCTGCTGTACATCTTCAAGCAGCCCTTCATGCGCCCAGTGCAGACCACTCA
GGAGGAGGACGGCTGCAGCTGCCGGTTTCCAGAGGAGGAAGAAGGAGGGTGTGAGCTGAGGGTGAAGTTTTCACGC
TCAGCTGACGCCCCTGCTTATCAGCAGGGCCAGAATCAGTTATATAATGAACTGAATCTGGGCAGGAGGGAGGAGT
ACGATGTGCTGGATAAACGGAGAGGCAGGGATCCCGAAATGGGAGGTAAGCCTCGGCGGAAGAATCCTCAGGAGGG
TCTATATAACGAACTCCAGAAGGATAAGATGGCCGAGGCTTACTCCGAGATCGGGATGAAAGGGGAGAGGCGCCGG
GGTAAAGGCCACGATGGCCTTTACCAGGGCCTCTCCACCGCAACAAAGGATACCTATGATGCCCTGCATATGCAGG
CCCTGCCTCCCCGG (SEQ ID NO: 135)
ATGGCACTACCCGTTACCGCTCTCTTGCTCCCTTTGGCCCTCCTCCTCCACGCCGCCAGACCCGATATCCAGATGA
CTCAGACTACATCTAGTTTGAGTGCATCTCTCGGAGATCGGGTCACCATTTCTTGTCGGGCTTCTCAGGATATCAG
TAAGTACCTTAATTGGTACCAGCAGAAGCCCGACGGGACAGTGAAGCTCCTGATCTACCACACCAGTAGGCTCCAC
TCTGGTGTCCCCTCCAGATTTAGTGGGTCAGGGAGTGGGACCGACTACTCCCTGACCATCTCCAATCTGGAGCAGG
AGGATATCGCCACCTACTTCTGCCAGCAGGGGAATACCCTGCCATATACATTTGGAGGGGGCACCAAGCTGGAGAT
TACTGGTGGTGGTGGATCAGGAGGTGGTGGTAGTGGAGGAGGAGGGTCAGAGGTGAAACTGCAGGAGAGCGGTCCT
GGTCTCGTTGCCCCAAGTCAGTCCCTGTCCGTGACATGTACCGTGTCTGGCGTGTCTCTGCCAGACTACGGAGTGT
CATGGATCAGACAGCCACCCAGGAAAGGCCTGGAGTGGCTGGGAGTGATTTGGGGCAGCGAGACCACATATTATAA
CTCCGCCCTGAAGTCCAGGCTGACCATCATCAAGGACAACTCCAAGAGTCAGGTGTTCTTGAAGATGAATAGCCTG
CAGACAGATGACACGGCCATCTACTACTGTGCGAAGCACTACTACTATGGCGGCAGCTACGCCATGGATTACTGGG
GGCAGGGGACATCTGTGACTGTGAGTTCAACAACCACTCCAGCTCCACGGCCTCCTACTCCAGCTCCAACCATTGC
AAGTCAGCCTCTGAGTCTGAGACCAGAGGCTTGTAGACCTGCAGCTGGTGGAGCTGTTCATACTCGCGGGCTGGAT
TTCGCCTGCGATATCTACATCTGGGCTCCTCTGGCCGGCACCTGCGGCGTCCTCCTCCTGAGCCTGGTGATCACTC
TGTACTGCAAGCGCGGAAGGAAGAAGCTGCTGTACATCTTCAAGCAGCCCTTCATGCGCCCAGTGCAGACCACTCA
GGAGGAGGACGGCTGCAGCTGCCGGTTTCCAGAGGAGGAGGAAGGAGGCTGTGAGCTGAGAGTGAAGTTTTCTCGC
AGCGCAGATGCCCCTGCCTACCAGCAGGGCCAGAATCAGTTATATAATGAACTGAATCTGGGCCGCAGGGAGGAGT
ATGATGTGCTGGATAAGCGGAGGGGCAGGGATCCCGAAATGGGGGGTAAGCCTCGGCGGAAGAATCCTCAGGAGGG
TCTATATAACGAACTCCAGAAGGATAAGATGGCCGAGGCTTACTCCGAGATCGGGATGAAAGGGGAGAGGCGCCGG
GGTAAAGGCCACGATGGCCTTTACCAGGGCCTCTCCACCGCAACAAAGGATACCTATGATGCCCTGCATATGCAGG
CCCTGCCCCCCCGG (SEQ ID NO: 136)
ATGGCCCTACCAGTGACAGCCCTCCTCCTCCCTTTGGCCTTGTTGCTCCACGCAGCAAGGCCAGATATTCAGATGA
CACAGACAACCAGCAGCCTCTCCGCCTCTCTTGGAGACAGGGTGACCATTTCTTGTCGGGCTTCTCAGGATATCAG
TAAGTACCTTAATTGGTACCAGCAGAAGCCCGACGGGACAGTGAAGTTGCTGATCTACCACACTAGCAGGCTGCAT
AGCGGTGTCCCTTCCAGATTCTCGGGATCTGGAAGCGGCACCGACTACAGCCTGACTATCAGCAACCTGGAGCAGG
AGGACATCGCCACATATTTCTGCCAGCAGGGGAATACCCTGCCCTATACATTTGGCGGGGGCACAAAGCTGGAGAT
TACAGGTGGCGGAGGCTCTGGAGGAGGAGGATCAGGCGGGGGCGGATCTGAAGTGAAACTTCAGGAATCCGGCCCC
GGCCTGGTCGCTCCTTCCCAGAGCCTGTCCGTCACCTGTACAGTCTCCGGCGTGTCCCTGCCAGATTATGGGGTGA
GTTGGATTCGCCAGCCCCCTAGAAAGGGGCTGGAATGGTTAGGCGTGATTTGGGGGTCAGAGACCACCTACTACAA
CTCTGCCCTCAAGTCACGCCTGACCATTATCAAGGACAATAGTAAGAGCCAGGTGTTTCTGAAGATGAATAGTCTT
CAGACCGATGACACCGCTATTTACTATTGTGCCAAGCACTACTACTATGGCGGCAGCTACGCCATGGATTACTGGG
GGCAGGGGACTAGTGTTACTGTGAGTAGCACTACTACCCCTGCCCCTAGACCTCCAACTCCAGCTCCAACCATTGC
AAGTCAGCCTCTGAGTCTGAGACCAGAGGCTTGTAGACCTGCAGCTGGTGGAGCTGTTCATACAAGGGGCCTGGAT
TTTGCCTGTGACATTTACATCTGGGCCCCTCTGGCAGGAACATGTGGCGTGCTCCTCCTGTCTCTGGTGATCACCC
TGTACTGTAAGAGAGGCAGGAAGAAGCTGCTGTACATCTTCAAGCAGCCATTCATGAGACCTGTGCAGACAACACA
GGAAGAGGATGGCTGCTCCTGCAGGTTCCCTGAGGAGGAGGAGGGGGGCTGTGAACTGCGCGTCAAATTCTCCCGG
TCTGCCGATGCCCCTGCTTATCAGCAGGGGCAGAATCAGTTATATAATGAACTGAATCTCGGCAGACGGGAGGAGT
ATGACGTGCTGGATAAGCGGAGGGGCAGGGATCCCGAAATGGGGGGTAAGCCTCGGCGGAAGAATCCTCAGGAGGG
TCTATATAACGAACTCCAGAAGGATAAGATGGCCGAGGCTTACTCCGAGATCGGGATGAAAGGGGAGAGGCGCCGG
GGTAAAGGCCACGATGGCCTTTACCAGGGCCTCTCCACCGCAACAAAGGATACCTATGATGCCCTGCATATGCAGG
CCCTGCCCCCCCGG (SEQ ID NO: 137)
ATGGCATTACCAGTTACCGCCCTCCTCCTCCCCCTCGCCCTCCTCCTCCACGCCGCCCGGCCCGACATCCAGATGA
CCCAGACCACCAGCAGCCTCAGCGCCAGCCTCGGCGACCGGGTGACCATCTCTTGCCGGGCCAGCCAGGACATCAG
CAAGTACCTCAATTGGTACCAGCAGAAGCCGGACGGCACCGTGAAGCTCCTGATCTACCACACCAGCCGGCTGCAC
TCCGGGGTGCCCAGCCGGTTCAGCGGCAGCGGCAGCGGCACCGACTACTCCCTGACCATCAGCAACCTGGAGCAGG
AGGACATCGCCACCTACTTCTGCCAGCAGGGGAACACCCTGCCCTACACCTTCGGCGGCGGCACCAAGCTGGAGAT
CACCGGCGGCGGCGGCAGCGGCGGCGGCGGCAGCGGCGGGGGCGGCAGCGAGGTGAAGCTGCAGGAGAGCGGCCCC
GGCCTGGTGGCCCCCAGCCAGAGCCTGTCCGTGACCTGCACCGTGTCCGGCGTGAGCCTGCCCGACTACGGCGTGT
CCTGGATCCGCCAGCCCCCCAGGAAGGGCCTGGAGTGGCTGGGCGTGATCTGGGGCAGCGAGACCACCTACTACAA
CAGCGCGCTGAAGTCCAGGCTGACCATCATCAAGGACAACAGCAAGAGCCAGGTGTTCCTGAAGATGAACAGCCTG
CAGACCGACGACACCGCCATCTACTACTGCGCCAAGCACTACTACTACGGCGGCAGCTACGCCATGGACTACTGGG
GCCAGGGCACCAGCGTGACCGTGTCCAGCACCACCACCCCCGCCCCCCGGCCCCCCACCCCCGCCCCCACCATCGC
CAGCCAGCCCCTGAGCCTGCGGCCGGAGGCCTGCAGGCCCGCCGCCGGCGGCGCCGTGCACACCCGGGGCCTGGAC
TTCGCCTGCGACATCTACATCTGGGCCCCCCTGGCCGGCACCTGCGGCGTGCTGCTGCTGAGCCTGGTGATCACCC
TGTACTGCAAGCGGGGCCGGAAGAAGCTGCTGTACATCTTCAAGCAGCCCTTCATGCGCCCCGTGCAGACCACCCA
GGAGGAGGACGGCTGCAGCTGCCGCTTCCCCGAGGAGGAGGAGGGCGGCTGCGAGCTGCGGGTGAAGTTCTCCCGC
AGCGCCGACGCCCCTGCGTACCAGCAGGGCCAGAACCAGCTGTACAACGAGCTGAACCTGGGCCGGCGCGAGGAGT
ACGACGTGCTGGACAAGCGCCGGGGCCGGGACCCCGAGATGGGCGGCAAGCCCCGGCGCAAGAACCCCCAGGAGGG
CCTGTACAACGAGCTGCAGAAGGACAAGATGGCCGAGGCCTACAGCGAGATCGGCATGAAGGGCGAGCGGCGGCGG
GGCAAGGGCCACGACGGCCTGTACCAGGGCCTGTCCACCGCCACCAAGGACACCTACGACGCCCTGCACATGCAGG
CCCTGCCCCCCCGG (SEQ ID NO: 138)
ATGGCCCTGCCCGTGACCGCCCTGCTGCTCCCCCTGGCCCTGCTGCTGCACGCAGCAAGGCCAGACATCCAGATGA
CCCAGACTACGAGCAGCCTGAGCGCCTCCCTGGGCGACAGGGTGACAATCAGCTGCCGGGCCAGTCAGGACATCAG
CAAGTACCTGAACTGGTACCAGCAGAAGCCTGACGGCACCGTGAAGCTGCTGATTTATCACACCTCCAGGCTGCAC
AGCGGCGTGCCCTCCAGATTCTCCGGCAGCGGCTCCGGCACCGACTACAGCCTGACCATCTCCAACCTGGAGCAGG
AGGATATCGCCACCTACTTCTGCCAGCAGGGGAACACCCTGCCCTATACCTTTGGGGGCGGAACCAAGCTGGAGAT
CACCGGCGGTGGCGGATCCGGCGGCGGCGGGAGCGGCGGCGGAGGATCTGAGGTCAAGCTGCAGGAGTCTGGCCCT
GGGCTGGTGGCCCCAAGCCAGAGCCTGTCCGTGACATGTACCGTGTCCGGCGTGTCTCTGCCTGACTACGGCGTGT
CATGGATCAGGCAGCCTCCTCGCAAGGGGCTGGAGTGGCTGGGCGTGATCTGGGGCAGCGAGACCACCTACTACAA
CAGTGCCCTGAAGTCCCGGCTGACCATCATCAAGGACAACAGCAAGAGCCAGGTGTTCTTGAAGATGAACAGCCTC
CAGACCGACGACACCGCCATTTACTACTGTGCCAAGCACTATTACTATGGCGGGTCCTACGCCATGGATTACTGGG
GCCAGGGCACCAGTGTGACCGTGTCCAGCACCACGACTCCTGCCCCACGCCCACCCACTCCAGCCCCTACCATCGC
CAGCCAGCCACTGAGCCTGCGGCCTGAGGCCTGCAGGCCAGCGGCTGGCGGGGCGGTGCACACCCGCGGGCTGGAC
TTCGCCTGCGACATCTACATCTGGGCCCCCCTCGCCGGCACCTGCGGCGTCCTCCTCCTGAGCCTGGTGATCACTC
TGTACTGCAAGAGGGGCCGCAAGAAGCTGCTGTATATCTTCAAGCAGCCTTTCATGCGGCCCGTGCAGACCACTCA
GGAGGAGGACGGCTGCAGCTGCCGGTTTCCTGAGGAGGAGGAGGGGGGCTGTGAGCTGCGGGTGAAGTTCAGCCGC
TCCGCCGACGCCCCCGCCTACCAGCAGGGCCAGAATCAGCTGTACAACGAGCTGAATCTGGGCCGCAGGGAGGAGT
ACGACGTGCTCGACAAGCGGCGGGGGCGGGACCCCGAGATGGGCGGCAAGCCCAGGCGGAAAAACCCCCAGGAGGG
GCTGTACAATGAGCTCCAGAAGGACAAAATGGCCGAGGCTTACAGCGAGATCGGCATGAAGGGGGAGAGGCGGAGG
GGCAAGGGGCATGACGGCCTGTACCAGGGGCTGTCCACAGCCACCAAGGACACCTACGACGCCCTGCACATGCAGG
CCCTGCCTCCTAGG (SEQ ID NO: 139)
ATGGCCCTGCCCGTGACCGCCCTGCTGCTTCCCCTGGCCCTGCTGCTGCACGCCGCAAGGCCAGACATCCAGATGA
CCCAGACTACCAGTTCTCTGTCCGCCTCTCTGGGCGACAGAGTGACGATCAGTTGCCGGGCCAGTCAGGACATCAG
CAAGTACCTGAACTGGTACCAGCAGAAGCCTGACGGCACCGTGAAGCTGCTGATCTACCACACCAGCCGGCTGCAC
AGCGGAGTGCCCAGCCGGTTCAGCGGCTCCGGGTCCGGCACTGATTACAGCCTGACCATCTCAAACCTGGAGCAGG
AGGACATCGCCACTTACTTCTGCCAGCAGGGCAATACCCTGCCCTACACCTTCGGAGGCGGCACCAAGCTGGAGAT
CACCGGCGGCGGCGGCTCCGGCGGCGGTGGCTCCGGCGGGGGCGGCTCCGAGGTGAAGCTGCAGGAGAGTGGCCCA
GGCCTGGTTGCCCCCAGCCAGAGCCTGAGCGTGACATGCACCGTGTCAGGCGTGAGTCTTCCTGACTACGGGGTGA
GCTGGATCAGGCAGCCCCCTAGGAAGGGCCTGGAGTGGCTGGGGGTGATCTGGGGCTCAGAGACCACATACTACAA
CTCTGCCCTCAAGAGCAGGCTGACCATCATCAAGGACAACTCCAAGTCCCAGGTGTTCTTGAAGATGAACAGCCTG
CAGACCGACGATACCGCCATTTACTACTGTGCCAAGCACTACTACTATGGCGGGTCCTACGCCATGGACTATTGGG
GGCAGGGGACTAGTGTGACTGTGAGCTCCACCACCACCCCAGCCCCCAGGCCCCCTACTCCTGCCCCTACCATCGC
CAGCCAGCCCCTGAGCCTGCGGCCTGAGGCCTGCAGGCCAGCCGCCGGGGGCGCCGTGCACACCAGGGGCCTGGAC
TTTGCCTGTGACATTTACATCTGGGCCCCTCTGGCCGGCACCTGCGGCGTGCTGCTGCTGAGCCTGGTGATCACCC
TGTACTGCAAGAGGGGCAGGAAGAAGCTGCTGTATATCTTCAAGCAGCCCTTCATGCGGCCAGTGCAGACCACTCA
GGAGGAGGACGGCTGCAGCTGCCGCTTCCCTGAGGAGGAGGAGGGCGGCTGCGAGCTGCGGGTGAAGTTCTCCCGC
AGCGCCGATGCCCCTGCCTACCAGCAGGGCCAGAATCAGCTGTACAATGAGCTGAATCTGGGCCGTCGGGAGGAGT
ACGACGTGCTGGATAAGCGCAGGGGCAGAGATCCTGAGATGGGCGGCAAGCCCCGCAGGAAGAATCCTCAGGAGGG
TCTGTACAATGAGCTGCAGAAGGACAAGATGGCTGAGGCTTATTCAGAGATCGGCATGAAGGGCGAGAGGCGGCGG
GGCAAGGGCCACGATGGCCTCTATCAGGGGCTGAGCACGGCCACCAAGGATACATACGACGCCCTGCACATGCAGG
CGCTGCCTCCCCGG (SEQ ID NO: 140)
ATGGCCCTCCCCGTCACCGCCCTGCTCCTCCCCCTGGCACTCCTGCTGCACGCCGCCAGGCCCGACATCCAGATGA
CCCAGACCACCAGCAGCCTCAGTGCCTCCCTGGGAGATAGGGTGACTATCTCCTGCAGGGCCAGTCAGGACATCAG
CAAGTACCTGAATTGGTACCAGCAGAAGCCTGACGGCACTGTGAAGCTGCTGATCTACCACACCTCCCGCCTGCAC
AGCGGTGTGCCCAGCCGGTTCTCCGGCTCTGGGTCCGGGACTGACTACAGCCTGACCATCTCCAACCTGGAGCAGG
AGGACATCGCCACCTACTTCTGCCAGCAGGGGAACACCCTGCCATACACCTTTGGTGGCGGGACCAAGCTGGAGAT
CACCGGCGGAGGCGGGAGCGGCGGGGGCGGCTCCGGCGGAGGCGGGAGCGAGGTCAAGCTGCAGGAGTCTGGCCCC
GGCCTGGTGGCCCCCAGCCAGAGCCTGAGCGTGACCTGCACCGTCTCCGGCGTGAGCCTGCCCGACTATGGCGTGA
GCTGGATCAGGCAGCCACCCAGGAAGGGCCTGGAGTGGCTGGGCGTGATCTGGGGGTCTGAGACCACCTACTATAA
CAGCGCCCTCAAGTCACGCCTGACCATCATCAAGGACAACAGCAAGTCCCAGGTGTTCCTGAAGATGAACAGTCTG
CAGACAGACGACACCGCCATCTACTACTGTGCCAAGCACTACTACTATGGCGGGTCTTACGCCATGGACTACTGGG
GGCAGGGGACGAGTGTGACTGTGTCCAGCACAACCACACCCGCCCCTAGGCCCCCAACACCCGCCCCCACTATCGC
AAGCCAGCCCCTGAGTCTGAGGCCCGAGGCCTGCCGGCCCGCCGCCGGCGGCGCCGTGCACACCAGGGGCCTGGAC
TTCGCTTGCGATATCTACATCTGGGCTCCTCTGGCCGGAACCTGCGGCGTGCTGCTGTTGTCCTTGGTGATCACTC
TGTACTGCAAGAGGGGCCGCAAGAAGCTGCTGTACATCTTTAAGCAGCCTTTCATGCGGCCCGTGCAGACCACCCA
GGAGGAGGACGGCTGCAGCTGCAGGTTCCCAGAGGAGGAGGAGGGCGGGTGTGAGCTGAGGGTGAAGTTCTCCAGG
TCTGCCGACGCCCCCGCCTACCAGCAGGGCCAGAATCAGCTCTACAATGAGCTGAATCTGGGCCGCCGGGAGGAGT
ACGATGTGCTCGACAAGCGGCGGGGGCGCGACCCAGAGATGGGCGGCAAGCCTAGGAGAAAGAACCCTCAGGAGGG
GCTCTACAATGAGCTGCAGAAGGACAAGATGGCTGAGGCGTATTCAGAGATCGGGATGAAAGGGGAGAGGAGGAGG
GGCAAGGGGCACGACGGCCTGTATCAGGGGCTGAGCACCGCCACCAAGGACACCTATGACGCACTGCACATGCAGG
CCCTGCCCCCGCGG (SEQ ID NO: 141)
ATGGCCCTGCCCGTGACCGCCCTGCTTCTGCCCCTGGCCCTGCTGCTCCACGCCGCCAGGCCAGACATCCAGATGA
CCCAGACAACCTCCAGCCTGAGCGCCAGCCTGGGCGACAGAGTGACCATCAGTTGCCGGGCCAGCCAGGACATCAG
CAAGTACCTGAATTGGTACCAGCAGAAGCCCGACGGCACCGTGAAGCTGCTGATCTACCACACCAGCCGCCTGCAC
AGCGGCGTGCCCAGCAGGTTCAGCGGCAGCGGCTCCGGCACCGACTACAGCCTGACCATCAGCAACCTGGAGCAGG
AGGACATCGCCACCTACTTCTGCCAGCAGGGCAATACCCTGCCCTACACCTTTGGCGGCGGCACCAAGCTGGAGAT
CACCGGCGGCGGCGGAAGCGGCGGCGGCGGCTCCGGCGGGGGCGGCAGCGAGGTGAAGCTGCAGGAGAGCGGCCCA
GGCCTGGTGGCCCCCAGCCAGAGCCTGAGCGTGACCTGCACCGTGAGCGGCGTGTCCCTGCCTGACTACGGGGTGA
GCTGGATCAGGCAGCCCCCCAGAAAGGGCCTGGAGTGGCTGGGCGTGATCTGGGGCAGCGAGACCACCTACTACAA
TTCCGCCCTGAAGTCCCGGCTGACCATCATCAAGGACAATTCCAAGAGCCAGGTGTTCCTGAAGATGAACTCCCTG
CAGACCGACGACACCGCCATCTACTACTGCGCCAAGCACTACTACTACGGCGGCTCCTACGCCATGGACTACTGGG
GCCAGGGCACCTCCGTGACCGTGAGCTCTACCACCACCCCCGCCCCTCGGCCCCCAACACCTGCCCCCACCATCGC
TAGCCAGCCCCTGTCTCTGAGACCCGAGGCTTGCAGACCAGCCGCCGGCGGCGCCGTGCACACCAGAGGCCTGGAC
TTCGCCTGCGACATCTACATCTGGGCCCCCCTGGCCGGCACCTGCGGCGTGCTGCTGCTGAGCCTGGTGATCACCC
TGTACTGCAAGAGGGGAAGGAAGAAGCTGCTGTACATCTTTAAGCAGCCTTTCATGAGACCCGTCCAGACCACACA
GGAGGAGGACGGCTGTAGCTGCAGGTTCCCAGAGGAGGAGGAGGGCGGCTGCGAGCTGCGGGTGAAGTTCAGCAGG
TCCGCTGACGCCCCCGCCTACCAGCAGGGCCAGAACCAGCTGTACAATGAGCTGAACCTGGGCCGGAGAGAGGAGT
ACGACGTGCTGGACAAGAGGAGAGGCAGGGACCCCGAGATGGGCGGCAAGCCTAGAAGGAAGAACCCCCAGGAGGG
ACTGTACAACGAGCTGCAGAAGGACAAGATGGCCGAGGCCTACTCTGAGATCGGCATGAAGGGCGAGAGGAGAAGA
GGCAAGGGCCACGATGGCCTGTACCAGGGCCTGAGCACCGCCACCAAGGACACCTACGACGCCCTGCACATGCAGG
CCCTGCCCCCCCGG (SEQ ID NO: 142)
ATGGCATTACCAGTTACCGCCCTCCTCCTCCCCCTCGCCCTCCTCCTCCACGCCGCCCGGCCAGATATCCAGATGA
CCCAGACCACCTCCTCCCTCAGCGCCTCCCTCGGCGACCGGGTGACCATCTCTTGCCGGGCCAGCCAGGACATCAG
CAAGTACCTCAATTGGTACCAGCAGAAGCCGGACGGCACCGTGAAGCTCCTGATCTACCACACCTCCCGCCTGCAC
AGCGGGGTGCCCAGCAGGTTCAGCGGCAGCGGCAGCGGCACCGACTACTCCCTGACCATCAGCAACCTGGAGCAGG
AGGACATCGCCACCTACTTCTGCCAGCAGGGGAACACCCTGCCCTACACCTTCGGCGGCGGCACCAAGCTGGAGAT
CACCGGGGGCGGGGGCAGCGGGGGCGGGGGCAGCGGCGGGGGCGGCAGCGAGGTGAAGCTGCAGGAGAGCGGCCCA
GGCCTGGTGGCCCCCAGCCAGAGCCTGTCCGTGACCTGCACCGTGAGCGGCGTGTCCCTGCCCGACTATGGCGTGA
GCTGGATCCGCCAGCCACCCAGGAAGGGCCTGGAGTGGCTGGGCGTGATCTGGGGCAGCGAGACCACCTACTACAA
CAGCGCCCTGAAGTCCAGGCTGACCATCATCAAGGACAACAGCAAGAGCCAGGTGTTCCTGAAGATGAACAGCCTG
CAGACCGACGACACCGCCATCTACTACTGCGCCAAGCACTACTACTACGGCGGCAGCTACGCCATGGACTACTGGG
GCCAGGGCACCTCCGTGACCGTGAGCAGCACCACCACCCCCGCCCCCCGCCCCCCCACCCCCGCCCCCACCATCGC
CAGCCAGCCGCTGAGCCTGCGGCCGGAGGCCTGCAGGCCCGCCGCCGGAGGCGCCGTGCACACCCGGGGCCTGGAC
TTCGCCTGCGACATCTACATCTGGGCCCCCCTGGCCGGCACCTGCGGCGTGCTGCTGCTGAGCCTGGTGATCACCC
TGTACTGCAAGCGGGGCAGGAAGAAGCTGCTGTACATCTTCAAGCAGCCCTTCATGCGGCCCGTGCAGACCACCCA
GGAGGAGGATGGCTGCAGCTGCAGGTTCCCTGAGGAGGAGGAGGGCGGCTGCGAGCTGCGGGTGAAGTTCTCCCGC
AGCGCCGACGCCCCCGCCTACCAGCAGGGCCAGAATCAGCTGTACAACGAGCTGAATCTGGGCCGGCGGGAGGAGT
ACGACGTGCTGGACAAGCGCCGGGGCCGGGACCCCGAGATGGGCGGCAAGCCCCGGCGCAAGAACCCCCAGGAGGG
CCTGTACAACGAGCTGCAGAAGGATAAGATGGCCGAGGCCTACAGCGAGATCGGCATGAAGGGCGAGCGGCGGAGG
GGGAAGGGCCACGACGGCCTGTATCAGGGCCTGTCCACCGCCACCAAGGACACCTACGACGCCCTGCACATGCAGG
CCCTGCCCCCCCGG (SEQ ID NO: 143)
ATGGCATTACCCGTGACGGCCCTCCTACTCCCCTTAGCTCTCTTGCTCCACGCCGCCCGCCCGGACATCCAGATGA
CCCAGACGACCTCGAGCTTGAGTGCGTCACTCGGGGACCGAGTGACGATCTCTTGCCGAGCGTCGCAGGACATCTC
GAAGTACTTGAATTGGTACCAGCAGAAGCCCGACGGGACCGTTAAGCTATTAATCTACCACACGAGTAGATTGCAT
AGCGGCGTTCCCAGTCGGTTCTCTGGTAGCGGTTCAGGTACTGACTACTCGCTGACCATCAGCAACCTGGAGCAGG
AGGACATCGCCACCTACTTCTGCCAGCAGGGTAACACGCTTCCCTACACCTTCGGTGGGGGGACCAAGCTGGAGAT
CACTGGTGGCGGAGGTAGCGGTGGCGGTGGTTCGGGCGGGGGCGGGAGCGAGGTGAAGCTGCAGGAGAGTGGGCCT
GGCCTCGTCGCGCCGTCACAGAGCCTCTCTGTGACGTGCACCGTGAGCGGGGTCAGCCTGCCGGACTACGGAGTGT
CCTGGATCCGGCAGCCGCCCCGCAAAGGCCTCGAGTGGCTCGGGGTCATTTGGGGGAGCGAGACAACGTACTACAA
TAGTGCGTTGAAGTCGCGACTTACTATTATCAAGGATAATAGTAAGTCGCAGGTCTTTCTGAAGATGAACTCACTT
CAGACTGACGACACCGCAATATACTATTGCGCCAAGCACTACTACTATGGAGGCAGTTACGCGATGGACTACTGGG
GGCAGGGGACATCCGTGACTGTCTCCAGCACCACCACGCCGGCCCCGCGGCCCCCCACCCCCGCGCCGACGATCGC
GTCTCAGCCCCTCAGCCTGAGGCCTGAGGCGTGTCGTCCAGCGGCGGGTGGGGCCGTGCACACACGGGGACTAGAC
TTTGCATGCGACATCTACATCTGGGCCCCGCTGGCGGGGACCTGTGGAGTGTTGCTGCTGAGTCTAGTCATCACCT
TGTACTGCAAGCGCGGCCGTAAGAAGCTCCTGTACATCTTCAAGCAGCCCTTCATGAGGCCTGTTCAAACGACGCA
GGAGGAGGACGGGTGCAGCTGCCGCTTCCCTGAGGAGGAGGAGGGCGGCTGCGAGCTGAGAGTGAAATTCTCACGC
TCGGCGGACGCCCCAGCTTATCAGCAGGGCCAGAACCAGCTGTACAATGAGCTGAACTTAGGCCGCAGGGAGGAGT
ACGACGTGCTCGACAAGCGCCGGGGTCGTGACCCGGAGATGGGTGGCAAGCCCCGGCGCAAGAATCCGCAGGAGGG
GCTCTACAACGAGCTCCAGAAGGACAAGATGGCGGAGGCCTACTCCGAGATTGGCATGAAGGGGGAGCGCCGTCGT
GGTAAGGGCCACGACGGCCTGTACCAGGGCCTGTCGACGGCCACTAAGGACACTTACGACGCCCTGCACATGCAGG
CGTTGCCGCCCCGG (SEQ ID NO: 144)
ATGGCATTACCAGTTACAGCCCTGTTGCTGCCCCTGGCCCTGCTGCTGCACGCCGCCCGGCCAGATATCCAGATGA
CGCAGACCACCAGCTCCCTGTCAGCCTCCCTGGGGGACAGGGTGACCATCAGCTGCCGGGCCAGCCAGGACATCAG
CAAGTACCTGAACTGGTACCAGCAGAAGCCTGATGGCACCGTGAAGCTGCTGATCTACCACACCTCCCGCCTGCAC
AGCGGGGTGCCCAGCAGGTTCAGCGGCAGCGGGTCAGGCACCGACTACAGCCTGACCATCTCCAACCTGGAGCAGG
AGGACATCGCCACCTACTTCTGCCAGCAGGGGAACACCCTGCCCTACACCTTCGGCGGGGGCACCAAGCTGGAGAT
CACTGGCGGCGGGGGGAGCGGGGGCGGGGGGTCTGGGGGCGGGGGTAGTGAGGTGAAGCTGCAGGAGAGCGGACCA
GGCCTGGTGGCCCCCAGCCAGAGCCTGTCCGTGACCTGCACCGTGTCTGGCGTGAGCCTGCCAGACTACGGCGTGT
CCTGGATCCGCCAGCCACCCAGGAAGGGCCTGGAGTGGCTGGGCGTGATCTGGGGCAGCGAGACCACCTACTACAA
CAGCGCGCTGAAGTCCAGGCTGACCATCATCAAGGACAACAGCAAGAGCCAGGTGTTCCTGAAGATGAACAGCCTG
CAGACCGACGACACCGCCATCTACTACTGTGCGAAGCACTACTACTATGGCGGGTCCTACGCCATGGATTACTGGG
GCCAGGGGACCTCAGTGACCGTGAGCAGCACCACTACCCCCGCCCCCAGACCCCCCACCCCCGCTCCCACCATCGC
CAGCCAGCCTCTGAGCCTGCGGCCAGAGGCCTGCAGGCCTGCCGCCGGGGGGGCCGTGCACACCCGCGGGCTGGAC
TTCGCCTGCGACATCTACATCTGGGCCCCCCTGGCCGGCACCTGCGGGGTGCTGCTGCTGAGCCTGGTGATCACCC
TGTACTGCAAGCGGGGCCGGAAGAAGCTGCTGTACATCTTCAAGCAGCCCTTCATGCGCCCCGTGCAGACCACCCA
GGAGGAGGACGGCTGCAGCTGCCGCTTCCCTGAGGAGGAGGAGGGCGGCTGCGAGCTGCGGGTGAAGTTCTCCCGC
AGCGCCGACGCCCCTGCGTACCAGCAGGGCCAGAATCAGCTGTACAACGAGCTGAATCTGGGCAGGCGGGAGGAGT
ACGACGTGCTGGACAAGCGCCGGGGCCGGGACCCCGAGATGGGGGGCAAGCCCCGGCGCAAGAACCCCCAGGAGGG
GCTGTACAACGAGCTGCAGAAGGATAAGATGGCCGAGGCCTATTCCGAGATCGGAATGAAGGGCGAGCGGCGGCGG
GGCAAGGGGCACGACGGGCTGTACCAGGGCCTGAGCACCGCCACCAAGGACACCTACGATGCCCTGCACATGCAGG
CCCTGCCCCCCCGG (SEQ ID NO: 145)
ATGGCTCTACCAGTGACAGCCCTCCTCCTCCCACTCGCCCTCCTCTTGCACGCCGCGCGCCCAGATATACAGATGA
CCCAGACGACGTCCAGCTTGAGTGCGTCACTCGGGGACCGAGTGACGATCTCTTGCCGGGCGTCGCAGGACATCTC
CAAGTACCTCAATTGGTACCAGCAGAAGCCAGACGGGACGGTGAAGCTCCTGATCTATCACACGTCCCGTCTGCAT
TCTGGTGTCCCGAGCCGGTTCTCCGGCTCGGGAAGTGGGACTGACTACAGTCTCACGATATCCAACCTGGAGCAGG
AGGATATCGCAACGTATTTCTGCCAGCAGGGAAATACGTTGCCGTACACCTTCGGGGGAGGGACAAAGCTGGAGAT
CACTGGTGGGGGCGGGAGTGGGGGGGGGGGGAGCGGGGGAGGGGGCAGCGAGGTCAAGCTCCAGGAGTCTGGGCCG
GGCCTCGTTGCCCCCTCCCAGTCACTCTCAGTCACTTGCACCGTTTCCGGGGTGTCGCTGCCGGACTACGGGGTGT
CGTGGATCCGGCAGCCACCCCGGAAAGGGCTGGAGTGGCTGGGAGTGATCTGGGGGTCCGAGACTACGTACTACAA
TAGTGCGTTGAAGTCTCGGCTCACCATAATAAAGGACAATAGTAAGTCGCAGGTCTTTCTGAAGATGAACAGTCTT
CAGACGGACGACACTGCGATTTACTATTGTGCCAAGCACTATTATTATGGTGGGTCGTACGCCATGGATTACTGGG
GTCAGGGGACCTCAGTGACCGTGAGCAGTACGACCACCCCCGCTCCCCGCCCCCCCACTCCCGCCCCCACCATAGC
ATCCCAGCCTTTGTCCCTCCGCCCGGAGGCGTGCCGCCCCGCCGCGGGCGGGGCGGTGCACACGCGAGGGCTGGAC
TTCGCGTGTGACATCTATATCTGGGCGCCGCTGGCGGGGACGTGCGGAGTTCTTCTGCTCTCGCTTGTAATCACGC
TGTATTGCAAGCGAGGGCGGAAGAAGCTCCTGTACATCTTCAAGCAGCCGTTCATGCGGCCGGTCCAGACGACCCA
AGAGGAGGACGGGTGCTCCTGCCGGTTCCCGGAGGAGGAGGAGGGGGGCTGTGAGCTGCGGGTCAAGTTCAGCCGT
TCAGCTGATGCCCCTGCGTACCAGCAGGGGCAGAATCAGCTGTACAACGAGCTGAACTTGGGCCGTCGAGAGGAGT
ACGACGTCCTCGACAAGCGTCGAGGACGTGATCCCGAGATGGGGGGTAAGCCCCGCCGTAAGAATCCTCAGGAGGG
GCTCTACAACGAGCTCCAGAAGGATAAGATGGCGGAGGCTTACTCCGAAATCGGGATGAAGGGAGAGCGCCGTCGT
GGCAAGGGCCACGACGGGCTCTACCAGGGCCTCTCGACGGCCACCAAGGACACCTACGACGCCCTGCACATGCAGG
CGTTGCCGCCGCGG (SEQ ID NO: 146)
ATGGCCCTACCCGTGACCGCTCTGCTGCTACCCCTGGCTCTGTTACTGCACGCCGCCCGCCCTGACATTCAGATGA
CTCAGACTACCAGCAGCCTGAGCGCCTCGTTGGGAGACCGCGTTACCATCTCCTGTCGCGCTAGCCAGGACATATC
TAAATATCTCAATTGGTACCAACAGAAGCCTGACGGCACAGTTAAACTTCTCATTTATCATACCTCACGCCTCCAT
TCCGGAGTGCCCAGTAGGTTCAGTGGAAGCGGCAGTGGAACCGACTATAGCCTCACTATTAGTAACTTGGAACAAG
AGGACATAGCAACATATTTTTGCCAACAGGGGAATACCTTGCCATATACATTCGGTGGGGGAACTAAACTCGAGAT
CACCGGCGGAGGTGGTAGCGGGGGAGGAGGATCAGGGGGAGGAGGTAGTGAGGTGAAGTTGCAGGAGTCAGGTCCG
GGACTGGTCGCGCCATCCCAGTCACTTTCAGTGACATGTACAGTGAGTGGTGTTTCACTGCCCGACTACGGAGTCT
CCTGGATCCGCCAGCCTCCCCGAAAGGGATTGGAGTGGCTTGGGGTAATATGGGGGAGCGAAACGACCTACTATAA
CTCAGCCCTCAAAAGTAGGCTGACAATTATAAAGGATAACAGTAAAAGTCAAGTTTTCCTTAAAATGAACAGCCTG
CAGACAGATGATACGGCCATCTACTACTGTGCGAAACATTACTACTACGGGGGCTCCTATGCCATGGATTATTGGG
GACAGGGAACCTCAGTCACTGTGTCATCTACAACCACTCCGGCTCCACGGCCTCCTACACCCGCACCCACTATTGC
CTCACAGCCTCTCAGCCTGAGACCTGAAGCCTGCAGACCCGCTGCGGGCGGCGCCGTACACACCCGAGGTCTGGAT
TTTGCCTGTGACATTTATATCTGGGCCCCGTTGGCTGGAACATGCGGTGTATTATTGCTGTCCCTGGTGATAACTT
TGTACTGTAAGCGGGGAAGGAAGAAGCTACTGTACATCTTCAAGCAGCCCTTTATGCGCCCTGTGCAAACAACTCA
AGAGGAGGATGGTTGTTCGTGCAGGTTCCCAGAGGAGGAGGAAGGCGGGTGTGAGTTGCGTGTTAAGTTTTCGAGA
TCAGCTGACGCCCCTGCTTACCAGCAAGGGCAGAACCAGCTCTACAACGAGCTGAATCTTGGACGTCGAGAAGAGT
ACGATGTTTTGGATAAAAGGCGTGGGCGCGATCCAGAGATGGGCGGTAAACCAAGGCGTAAGAATCCTCAGGAGGG
TCTCTATAACGAGTTGCAGAAGGATAAGATGGCTGAGGCTTACAGCGAAATCGGAATGAAAGGAGAGAGACGGCGA
GGTAAAGGTCACGATGGTCTCTATCAGGGATTGAGCACAGCGACCAAGGACACGTACGACGCCCTTCACATGCAAG
CGCTGCCTCCGAGG (SEQ ID NO: 147)
ATGGCTCTGCCAGTAACCGCGCTTCTACTGCCATTAGCCTTGCTGCTCCACGCAGCCCGACCCGACATTCAGATGA
CTCAGACTACTAGCTCTCTGAGTGCATCGCTAGGTGATCGTGTTACGATTTCCTGCCGGGCCTCACAGGATATTTC
TAAATACCTGAACTGGTACCAACAGAAGCCCGATGGGACGGTGAAGTTATTGATCTACCACACCAGTCGGTTGCAC
TCCGGCGTCCCATCCAGGTTTTCCGGGTCAGGCAGCGGTACTGACTATAGCCTTACCATCAGCAACCTGGAACAGG
AAGACATAGCCACCTACTTCTGCCAGCAAGGCAACACACTGCCCTACACCTTTGGTGGCGGAACCAAGCTGGAGAT
TACTGGGGGCGGGGGTTCTGGTGGTGGTGGTTCGGGCGGGGGGGGGAGTGAGGTCAAGCTTCAGGAATCGGGACCT
GGTCTAGTGGCTCCATCTCAGTCCCTGTCCGTGACTTGCACTGTGTCCGGCGTGTCGTTGCCGGACTACGGTGTCT
CGTGGATCAGGCAACCCCCCCGCAAGGGCCTCGAGTGGCTAGGGGTGATTTGGGGCTCCGAAACTACCTACTACAA
CTCCGCGCTCAAGTCTAGGCTGACGATCATCAAGGACAACTCTAAATCCCAAGTGTTCTTAAAGATGAACAGTCTG
CAAACAGACGATACCGCGATATATTATTGCGCTAAGCATTATTATTATGGTGGCTCCTACGCCATGGATTACTGGG
GTCAAGGCACTTCCGTTACAGTCAGTTCAACAACAACCCCAGCTCCACGTCCTCCAACACCAGCCCCGACCATCGC
CTCTCAGCCACTGTCCCTCAGGCCTGAGGCCTGCCGGCCTGCGGCCGGAGGGGCAGTGCATACGCGGGGGTTGGAC
TTTGCTTGCGACATTTATATCTGGGCGCCCCTGGCCGGTACATGTGGAGTGCTCCTTTTGTCCCTGGTGATCACCT
TGTACTGCAAGAGAGGGAGAAAGAAGCTACTCTACATCTTTAAGCAGCCGTTCATGCGACCGGTCCAGACCACCCA
GGAAGAGGACGGCTGCAGCTGCCGGTTTCCTGAAGAGGAAGAAGGCGGCTGCGAATTACGCGTGAAGTTTTCGCGC
AGCGCCGATGCTCCCGCTTATCAGCAAGGCCAAAACCAGCTCTACAATGAGTTGAACCTGGGGCGACGTGAGGAGT
ATGATGTTCTTGATAAGCGGCGGGGCCGAGACCCTGAGATGGGCGGGAAACCGCGACGTAAGAATCCTCAGGAGGG
GCTGTACAACGAGCTCCAGAAAGATAAGATGGCGGAAGCGTATAGCGAGATCGGTATGAAGGGGGAGCGCCGGCGC
GGGAAGGGTCACGATGGACTGTACCAGGGCCTCAGTACCGCGACAAAGGATACCTATGACGCGTTGCATATGCAGG
CCCTGCCCCCAAGG (SEQ ID NO: 148)
ATGGCCCTGCCTGTGACCGCCCTGCTGCTGCCACTGGCCCTGCTGCTGCACGCCGCCCGGCCAGATATCCAGATGA
CACAGACAACCTCCAGCCTGAGCGCCAGCCTGGGGGACAGAGTGACCATCAGCTGCCGGGCCTCCCAAGACATCAG
CAAATACCTGAACTGGTACCAGCAGAAACCCGATGGCACCGTGAAGCTGCTGATCTACCACACTTCCAGGCTGCAC
AGTGGGGTGCCCTCCCGGTTCTCCGGCAGCGGCAGCGGCACCGACTACAGCCTGACCATCAGCAACCTGGAGCAGG
AGGACATCGCCACCTACTTTTGCCAGCAGGGCAACACCCTGCCCTACACCTTCGGCGGCGGCACCAAGCTGGAAAT
CACCGGGGGCGGGGGATCTGGGGGCGGCGGCTCCGGCGGCGGAGGATCCGAGGTGAAGCTGCAGGAGTCCGGCCCA
GGCCTGGTGGCCCCCAGCCAAAGTCTGTCCGTGACCTGTACCGTGTCCGGCGTGTCCCTGCCCGACTACGGCGTGA
GCTGGATTCGCCAGCCCCCCCGGAAGGGCCTGGAGTGGCTGGGCGTGATCTGGGGCAGCGAGACCACCTATTACAA
CAGCGCCCTGAAGTCCAGGCTGACCATCATTAAAGATAATAGCAAGAGCCAAGTGTTCCTCAAGATGAATAGCCTG
CAGACCGACGATACCGCCATCTACTACTGTGCCAAACACTACTACTACGGCGGGAGCTACGCCATGGACTACTGGG
GCCAAGGCACCTCCGTGACCGTGAGCAGCACCACCACCCCAGCCCCCAGGCCCCCTACCCCCGCTCCTACAATTGC
CAGCCAGCCACTGTCCCTGCGCCCTGAGGCCTGCCGGCCCGCCGCCGGCGGCGCCGTGCATACCCGGGGCCTGGAC
TTCGCCTGTGACATCTACATCTGGGCCCCCCTGGCTGGGACCTGCGGCGTGCTGCTGCTGAGCCTGGTGATCACAC
TGTACTGCAAAAGGGGCCGGAAAAAACTGCTGTACATCTTCAAGCAGCCCTTCATGAGACCCGTGCAGACCACCCA
GGAGGAGGACGGATGCTCCTGCCGCTTCCCAGAGGAGGAGGAGGGCGGGTGCGAGCTGAGAGTGAAGTTTAGCCGC
AGCGCCGATGCTCCCGCCTACCAGCAGGGCCAGAATCAGCTGTATAACGAACTCAACCTGGGCAGAAGAGAAGAGT
ACGATGTGCTGGATAAAAGACGGGGCAGGGACCCCGAGATGGGCGGCAAGCCAAGAAGAAAGAATCCCCAGGAGGG
CCTGTACAACGAGCTGCAGAAGGATAAGATGGCCGAAGCCTACAGCGAGATTGGCATGAAGGGAGAGCGGAGGCGC
GGCAAGGGCCACGACGGCCTCTACCAGGGCCTGTCCACCGCCACCAAGGATACCTACGACGCTCTGCACATGCAAG
CTCTGCCCCCCCGC (SEQ ID NO: 149)
ATGGCCCTGCCCGTCACCGCCCTGCTGCTGCCCCTGGCCCTGCTGCTGCACGCCGCCCGGCCCGATATCCAAATGA
CACAGACCACCAGCTCCCTGAGCGCCAGCCTGGGCGACAGAGTGACCATCTCCTGCAGGGCTAGCCAGGACATCAG
CAAGTATCTGAACTGGTACCAGCAGAAGCCCGACGGCACCGTGAAGCTCCTGATCTATCACACCTCCAGGCTGCAC
AGCGGCGTGCCCAGCAGATTCTCCGGCAGCGGCAGCGGCACCGACTACTCCCTGACCATTAGCAACCTGGAGCAAG
AGGACATCGCCACCTACTTCTGCCAGCAGGGCAACACACTGCCCTATACCTTTGGCGGCGGCACCAAGCTGGAGAT
CACCGGCGGCGGCGGCAGCGGCGGCGGCGGCAGCGGCGGCGGCGGCTCCGAGGTGAAACTGCAGGAAAGCGGCCCC
GGCCTGGTGGCCCCCTCCCAGAGCCTGAGCGTGACCTGCACCGTGAGCGGCGTGAGCCTGCCTGACTACGGCGTGT
CCTGGATTAGGCAACCCCCCAGGAAGGGACTGGAGTGGCTCGGCGTGATTTGGGGAAGCGAGACCACCTACTACAA
CTCCGCCCTGAAGTCCAGACTCACCATCATTAAAGATAACAGCAAATCCCAAGTGTTCCTGAAGATGAACTCCCTG
CAAACCGACGACACCGCCATCTACTACTGCGCCAAGCACTACTACTACGGAGGCAGCTACGCCATGGACTATTGGG
GCCAGGGCACCAGCGTGACCGTGAGCAGCACTACAACCCCCGCCCCCAGACCTCCTACCCCTGCCCCCACCATCGC
CTCACAGCCTCTGAGCCTGAGACCCGAGGCCTGCAGGCCCGCCGCCGGCGGCGCCGTGCACACCCGGGGACTGGAC
TTCGCCTGCGACATTTACATCTGGGCCCCCCTGGCAGGCACCTGCGGCGTGCTCCTGCTGAGCCTGGTGATTACCC
TGTACTGCAAGAGAGGAAGAAAGAAACTGCTGTACATCTTCAAGCAGCCCTTCATGAGGCCCGTGCAGACCACCCA
GGAAGAGGACGGCTGCTCCTGTAGGTTCCCCGAGGAGGAGGAGGGCGGCTGCGAGCTCAGAGTGAAGTTTAGCAGG
AGCGCCGACGCCCCCGCTTACCAGCAAGGCCAGAACCAGCTGTACAATGAACTGAACCTGGGCAGACGGGAGGAAT
ACGACGTGCTGGACAAAAGGAGAGGAAGAGACCCCGAGATGGGCGGAAAGCCCCGGCGCAAGAACCCACAGGAGGG
ACTGTACAACGAGCTGCAAAAAGACAAGATGGCCGAAGCTTACAGCGAGATCGGAATGAAGGGCGAGAGGAGACGC
GGCAAGGGACACGATGGCCTCTACCAGGGCCTGAGCACCGCCACCAAAGACACCTATGATGCCCTCCACATGCAAG
CCCTGCCCCCTAGG (SEQ ID NO: 150)
ATGGCCTTACCAGTGACCGCCTTGCTCCTGCCGCTGGCCTTGCTGCTCCACGCCGCCAGGCCGGACATCCAGATGA
CACAGACTACATCCTCCCTGTCTGCCTCTCTGGGAGACAGAGTCACCATCAGTTGCAGGGCAAGTCAGGACATTAG
TAAATATTTAAATTGGTATCAGCAGAAACCAGATGGAACTGTTAAACTCCTGATCTACCATACATCAAGATTACAC
TCAGGAGTCCCATCAAGGTTCAGTGGCAGTGGGTCTGGAACAGATTATTCTCTCACCATTAGCAACCTGGAGCAAG
AAGATATTGCCACTTACTTTTGCCAACAGGGTAATACGCTTCCGTACACGTTCGGAGGGGGGACCAAGCTGGAGAT
CACAGGTGGCGGTGGCTCGGGCGGTGGTGGGTCGGGTGGCGGCGGATCTGAGGTGAAACTGCAGGAGTCAGGACCT
GGCCTGGTGGCGCCCTCACAGAGCCTGTCCGTCACATGCACTGTCTCAGGGGTCTCATTACCCGACTATGGTGTAA
GCTGGATTCGCCAGCCTCCACGAAAGGGTCTGGAGTGGCTGGGAGTAATATGGGGTAGTGAAACCACATACTATAA
TTCAGCTCTCAAATCCAGACTGACCATCATCAAGGACAACTCCAAGAGCCAAGTTTTCTTAAAAATGAACAGTCTG
CAAACTGATGACACAGCCATTTACTACTGTGCCAAACATTATTACTACGGTGGTAGCTATGCTATGGACTACTGGG
GCCAAGGAACCTCAGTCACCGTCTCCTCAACCACGACGCCAGCGCCGCGACCACCAACACCGGCGCCCACCATCGC
GTCGCAGCCCCTGTCCCTGCGCCCAGAGGCGTGCCGGCCAGCGGCGGGGGGCGCAGTGCACACGAGGGGGCTGGAC
TTCGCCTGTGATATCTACATCTGGGCGCCCTTGGCCGGGACTTGTGGGGTCCTTCTCCTGTCACTGGTTATCACCC
TTTACTGCAAACGGGGCAGAAAGAAACTCCTGTATATATTCAAACAACCATTTATGAGACCAGTACAAACTACTCA
AGAGGAAGATGGCTGTAGCTGCCGATTTCCAGAAGAAGAAGAAGGAGGATGTGAACTGAGAGTGAAGTTCAGCAGG
AGCGCAGACGCCCCCGCGTACCAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGAGAAGGAAGAACCCTCAGGAAGG
CCTGTACAATGAACTGCAGAAAGATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACCTACGACGCCCTTCACATGCAGG
CCCTGCCCCCTCGC (SEQ ID NO: 151)
ATGGCACTTCCCGTTACCGCCCTCCTCCTTCCACTCGCCCTCCTCCTCCACGCCGCCCGCCCAGATATACAGATGA
CACAGACCACCTCCAGCTTGAGTGCCTCCCTCGGGGACAGGGTGACCATCTCTTGTCGGGCTTCTCAGGACATCAG
CAAGTACCTCAATTGGTACCAGCAGAAGCCCGACGGGACCGTGAAGCTCCTGATCTACCACACCAGCAGGCTGCAC
AGCGGCGTGCCCTCCAGGTTTAGCGGGTCAGGGAGTGGGACCGACTACTCCCTGACCATCTCAAACCTGGAGCAGG
AGGACATCGCCACCTACTTTTGCCAGCAGGGCAACACCCTGCCCTACACCTTCGGGGGAGGCACTAAGCTGGAGAT
CACTGGCGGTGGTGGGAGCGGGGGTGGGGGGTCTGGGGGCGGGGGTAGCGAGGTGAAGCTGCAGGAGTCCGGACCC
GGCCTGGTCGCCCCCAGCCAGTCCCTGTCCGTGACATGCACCGTGTCTGGCGTGAGCCTGCCAGACTACGGCGTGT
CATGGATCAGGCAGCCCCCCCGGAAGGGGCTGGAGTGGCTGGGGGTGATCTGGGGGTCCGAGACTACCTACTACAA
CAGCGCGCTGAAGTCCAGGCTGACCATCATCAAGGACAACTCCAAGAGTCAGGTGTTCTTGAAGATGAACAGCCTG
CAGACCGATGACACCGCCATATACTACTGTGCGAAGCACTACTACTATGGCGGGTCATACGCCATGGATTACTGGG
GCCAGGGGACCTCAGTGACCGTGAGCAGCACCACCACCCCCGCCCCCAGACCCCCCACCCCCGCTCCCACCATCGC
CAGCCAGCCCCTGAGCCTGCGCCCCGAGGCCTGCCGGCCCGCGGCCGGCGGCGCCGTGCACACTCGGGGGCTGGAC
TTCGCCTGTGACATCTATATCTGGGCCCCTCTGGCCGGCACCTGCGGCGTCCTCCTCCTGAGTCTGGTGATCACCC
TGTACTGCAAGCGCGGAAGGAAGAAGCTGCTGTACATCTTCAAGCAGCCCTTCATGCGCCCAGTGCAGACCACTCA
GGAGGAGGACGGCTGCAGCTGCCGGTTCCCAGAGGAGGAGGAGGGCGGGTGCGAGCTGCGGGTGAAGTTCTCCCGC
AGCGCCGATGCCCCTGCGTACCAGCAGGGGCAGAATCAGCTCTACAACGAGCTGAACCTGGGCCGCCGCGAGGAGT
ACGACGTGCTCGACAAGCGGCGGGGCCGGGATCCCGAGATGGGGGGTAAGCCTCGGCGGAAGAATCCTCAGGAGGG
GCTCTACAACGAGCTCCAGAAGGATAAGATGGCCGAGGCTTACTCCGAGATCGGGATGAAGGGGGAGCGGCGGCGG
GGCAAGGGGCACGACGGCCTGTATCAGGGCCTGAGTACCGCCACCAAGGATACTTACGACGCCCTGCACATGCAGG
CCCTGCCCCCCCGG (SEQ ID NO: 152)
ATGGCCCTACCAGTCACCGCCCTCCTCCTCCCCTTGGCCCTCCTCCTCCACGCCGCCCGGCCAGATATTCAGATGA
CACAGACCACCTCCAGCCTTAGTGCCTCCCTCGGAGATAGGGTGACCATTTCTTGTCGGGCTTCTCAGGACATCAG
CAAGTACCTCAATTGGTACCAGCAGAAGCCCGACGGGACAGTGAAGTTGCTGATCTACCACACTAGCAGGCTGCAT
AGTGGCGTGCCCTCCCGCTTCTCTGGAAGCGGGAGCGGCACCGACTACAGCCTGACTATCAGCAACCTGGAGCAGG
AGGACATCGCCACCTACTTCTGCCAGCAGGGGAATACCCTGCCCTATACCTTCGGGGGAGGCACTAAGCTGGAGAT
TACTGGCGGAGGCGGCAGTGGAGGAGGAGGGTCAGGGGGAGGAGGATCTGAAGTGAAACTTCAGGAGAGTGGGCCC
GGCCTTGTTGCTCCTTCTCAGAGCCTGTCCGTGACATGTACCGTGTCTGGCGTGTCTCTGCCAGACTACGGCGTGT
CATGGATCAGGCAGCCTCCTAGGAAGGGGCTGGAGTGGCTGGGCGTGATCTGGGGTAGTGAGACTACTTACTACAA
TAGTGCCCTGAAGTCCCGGCTGACTATTATCAAGGATAATAGCAAGTCTCAGGTGTTCCTGAAGATGAACAGCCTG
CAGACAGATGACACGGCCATCTACTACTGTGCGAAGCACTACTACTATGGCGGCAGCTACGCCATGGATTACTGGG
GCCAGGGCACTAGTGTGACAGTGAGTAGTACCACTACCCCAGCTCCACGCCCACCTACTCCAGCCCCTACAATTGC
CTCCCAGCCTCTGTCTCTGCGCCCTGAAGCTTGTCGCCCTGCTGCTGGTGGAGCTGTGCACACCAGAGGGCTGGAC
TTCGCCTGCGACATCTATATCTGGGCTCCTCTGGCCGGCACCTGCGGCGTCCTCCTCCTGAGTCTGGTGATCACCC
TGTACTGCAAGCGCGGAAGGAAGAAGCTGCTGTACATCTTCAAGCAGCCCTTCATGCGCCCAGTGCAGACCACTCA
GGAGGAGGACGGCTGCAGCTGCCGGTTTCCAGAGGAGGAAGAGGGTGGATGTGAGCTGCGGGTGAAGTTCAGCCGC
TCTGCTGATGCACCAGCCTACCAGCAGGGCCAGAATCAGCTCTACAACGAGCTGAATCTGGGCCGCAGGGAGGAGT
ACGACGTGCTCGACAAGCGCAGGGGCAGAGATCCAGAGATGGGAGGCAAACCTAGGAGGAAGAATCCCCAGGAGGG
GCTCTACAACGAGCTGCAGAAGGACAAGATGGCCGAGGCCTACTCTGAGATCGGAATGAAGGGCGAGAGGAGGCGG
GGAAAAGGCCACGATGGCCTGTATCAGGGGCTGTCCACCGCCACCAAGGACACCTATGATGCCCTGCACATGCAGG
CCCTGCCTCCTCGG (SEQ ID NO: 153)
ATGGCTCTCCCAGTGACCGCCCTCCTCCTTCCCCTTGCCCTCCTCTTGCACGCCGCTAGACCCGACATCCAGATGA
CTCAGACTACCTCTAGTTTGAGTGCATCCTTGGGTGATCGGGTCACCATCTCTTGTCGGGCTTCTCAGGATATCAG
TAAGTACCTTAATTGGTATCAGCAGAAGCCCGACGGGACCGTGAAGCTCTTGATCTACCATACATCAAGGCTTCAC
AGCGGCGTGCCCTCCAGGTTTAGCGGGTCAGGGAGTGGGACCGACTACTCCCTGACCATCTCAAACCTGGAGCAGG
AGGACATCGCTACTTACTTCTGCCAGCAGGGTAACACCCTGCCCTACACCTTTGGGGGAGGGACAAAGCTGGAGAT
CACTGGTGGGGGCGGGAGTGGGGGGGGGGGGAGCGGGGGTGGGGGCAGCGAGGTCAAGCTCCAGGAGTCTGGGCCT
GGCCTCGTTGCCCCATCCCAGAGTCTGTCAGTGACATGCACTGTGAGTGGCGTGTCACTGCCAGACTATGGGGTGT
CCTGGATCCGCCAGCCACCCAGGAAGGGCCTGGAGTGGCTGGGCGTGATCTGGGGGAGTGAGACCACCTACTATAA
TAGTGCTTTGAAGTCTAGACTTACTATTATCAAGGATAATAGTAAGTCTCAGGTCTTTCTGAAGATGAACAGTCTT
CAGACAGACGACACCGCAATATACTATTGCGCAAAGCACTATTATTATGGGGGGTCTTACGCCATGGACTACTGGG
GTCAGGGAACGTCCGTGACCGTCAGTAGTACCACCACCCCCGCTCCCCGCCCCCCCACTCCCGCCCCCACCATTGC
ATCCCAGCCTTTGTCCCTCCGCCCAGAGGCCTGTAGGCCAGCCGCCGGGGGTGCGGTGCACACCCGGGGGCTGGAC
TTCGCATGTGACATCTACATATGGGCTCCTCTGGCCGGCACCTGCGGCGTCCTCCTCCTGAGTCTGGTGATCACCC
TGTACTGCAAGCGCGGAAGGAAGAAGCTGCTGTACATCTTCAAGCAGCCCTTCATGCGCCCAGTGCAGACCACTCA
GGAGGAGGACGGCTGCAGCTGCCGGTTCCCAGAGGAGGAGGAGGGGGGGTGTGAGCTGAGGGTGAAGTTTTCACGC
TCAGCTGACGCCCCCGCCTACCAGCAGGGCCAGAATCAGCTCTACAACGAGCTGAATCTGGGCCGGCGGGAGGAGT
ACGATGTCCTCGACAAGAGGAGGGGGAGGGATCCCGAGATGGGGGGTAAGCCTCGGCGGAAGAATCCTCAGGAGGG
GCTCTACAACGAGCTCCAGAAGGATAAGATGGCCGAGGCTTACTCCGAGATCGGGATGAAGGGGGAGCGGCGGAGG
GGGAAGGGCCACGATGGACTGTATCAGGGGTTGTCCACTGCCACAAAGGACACCTATGATGCACTCCATATGCAGG
CCCTGCCCCCTCGG (SEQ ID NO: 154)
ATGGCTCTCCCAGTTACAGCCCTCTTGTTGCCTTTGGCCTTGCTCTTGCACGCCGCAAGGCCCGATATCCAGATGA
CCCAGACCACCAGTTCCCTAAGCGCCAGCTTGGGGGACCGGGTGACCATCTCTTGTCGGGCCAGTCAGGATATCAG
CAAGTACCTCAATTGGTACCAGCAGAAGCCGGACGGCACCGTGAAGTTGCTGATCTATCACACCAGTAGGCTGCAC
TCCGGCGTGCCCTCCCGGTTCTCCGGGAGCGGCAGCGGGACAGACTACAGTCTGACCATCAGCAACCTGGAGCAGG
AGGACATTGCCACCTACTTCTGCCAGCAGGGGAACACCCTGCCCTATACATTTGGCGGGGGCACCAAGCTGGAGAT
TACAGGCGGGGGAGGCTCTGGCGGCGGGGGCAGCGGGGGGGGCGGCTCTGAGGTGAAGCTTCAGGAGTCCGGCCCC
GGCCTCGTTGCCCCCAGCCAGAGCCTCTCCGTGACCTGTACCGTCTCCGGCGTGTCCCTGCCAGATTATGGGGTGT
CCTGGATCCGCCAGCCACCCCGGAAGGGGCTGGAGTGGCTGGGCGTGATCTGGGGCTCAGAGACAACCTACTACAA
CTCTGCCCTGAAGAGTAGACTTACTATTATCAAGGATAATAGTAAGTCTCAGGTGTTTCTGAAGATGAATAGCCTG
CAGACAGATGACACGGCCATCTACTACTGCGCTAAGCACTACTACTATGGCGGCAGCTATGCCATGGACTACTGGG
GCCAGGGGACTAGCGTGACAGTGTCCTCCACCACCACCCCTGCTCCCAGGCCCCCCACGCCTGCCCCCACCATCGC
GAGCCAGCCACTGTCCCTCCGGCCAGAGGCCTGCCGGCCTGCTGCCGGAGGGGCAGTGCACACAAGAGGGCTGGAT
TTCGCCTGTGACATTTACATCTGGGCTCCTCTGGCCGGCACCTGCGGCGTCCTCCTCCTGAGCCTGGTGATCACCC
TCTACTGCAAGAGGGGCAGGAAGAAGCTGCTGTACATCTTCAAGCAGCCTTTCATGAGGCCCGTGCAGACCACCCA
GGAGGAGGACGGCTGCAGCTGCCGGTTCCCAGAGGAGGAGGAGGGCGGCTGCGAGCTGCGGGTGAAGTTCTCCCGC
AGCGCAGACGCCCCCGCCTACCAGCAGGGCCAGAATCAGCTGTACAACGAGCTGAATCTGGGCCGGCGGGAGGAGT
ACGACGTGCTGGACAAGCGCCGGGGCCGGGACCCCGAGATGGGGGGCAAGCCCCGGCGCAAGAATCCTCAGGAGGG
GCTGTACAACGAGCTCCAGAAGGATAAGATGGCGGAGGCCTACAGCGAGATCGGGATGAAGGGCGAGCGCAGGCGG
GGCAAGGGGCATGACGGGCTGTACCAGGGCCTGTCAACTGCCACCAAGGACACCTACGACGCCCTGCACATGCAGG
CCCTGCCTCCCCGG (SEQ ID NO: 155)
ATGGCCTTACCAGTTACGGCGCTATTGCTCCCGTTGGCGCTATTGCTCCACGCCGCGCGCCCGGACATACAGATGA
CGCAGACGACGTCTTCGTTATCGGCGAGTTTGGGCGACCGGGTTACGATATCGTGCAGGGCTTCGCAGGACATAAG
TAAGTACCTTAATTGGTACCAGCAGAAGCCCGACGGTACGGTAAAACTACTCATCTATCATACGTCGCGTTTGCAT
AGTGGAGTACCCTCCCGTTTTTCGGGGAGTGGGTCGGGTACCGACTATTCGCTAACGATATCGAACTTGGAGCAAG
AAGATATCGCGACGTACTTTTGCCAGCAAGGGAATACGTTACCGTACACGTTTGGTGGAGGTACGAAACTAGAGAT
CACGGGTGGTGGTGGTTCGGGTGGTGGTGGTTCGGGTGGGGGCGGCTCGGAAGTGAAACTTCAAGAGTCGGGTCCC
GGTCTAGTGGCGCCGAGCCAATCGCTATCGGTAACGTGTACGGTTTCGGGTGTATCGCTACCGGATTACGGTGTAT
CGTGGATACGCCAACCGCCGCGTAAGGGCCTAGAATGGCTAGGGGTTATATGGGGTAGTGAGACTACCTATTATAA
CTCGGCGTTGAAATCGCGTCTAACGATAATAAAAGATAATTCAAAATCGCAGGTTTTTCTAAAAATGAATTCGCTA
CAAACGGATGATACGGCGATATATTATTGTGCGAAGCACTATTATTATGGTGGTTCCTATGCGATGGATTATTGGG
GTCAAGGTACGTCGGTAACCGTGTCGTCGACGACGACGCCGGCGCCGCGCCCGCCGACGCCGGCGCCGACGATAGC
GTCGCAACCGCTATCGTTGCGGCCGGAGGCGTGCCGCCCGGCGGCGGGCGGGGCGGTGCATACGCGCGGCCTCGAT
TTTGCGTGTGACATTTACATATGGGCGCCGCTAGCGGGCACGTGTGGTGTCCTACTACTATCGTTGGTGATCACCC
TATATTGTAAGCGTGGCAGGAAGAAGCTTCTTTATATATTTAAACAGCCGTTTATGCGGCCGGTACAAACGACGCA
AGAAGAAGATGGTTGTTCGTGCCGCTTCCCGGAAGAAGAAGAAGGTGGCTGTGAGCTACGCGTAAAGTTTTCGCGT
TCGGCGGATGCTCCGGCGTATCAACAAGGTCAAAATCAGTTGTATAACGAACTGAATCTAGGGCGTCGTGAAGAAT
ACGACGTCCTAGATAAACGTCGTGGTCGGGACCCGGAAATGGGTGGTAAGCCGCGTCGTAAAAACCCGCAAGAGGG
GTTATATAATGAACTCCAAAAGGATAAAATGGCGGAGGCGTATTCGGAGATCGGGATGAAAGGCGAGCGTCGCCGT
GGTAAGGGCCACGACGGCCTTTATCAAGGTCTCTCGACGGCGACGAAGGATACGTATGATGCGTTGCATATGCAAG
CGTTGCCGCCGCGT (SEQ ID NO: 156)
ATGGCACTACCGGTAACGGCGCTACTACTACCGCTAGCGTTACTATTGCATGCAGCACGTCCGGATATACAAATGA
CGCAAACAACTTCGAGTTTATCGGCATCTTTAGGAGACCGTGTAACCATATCGTGTCGAGCGTCTCAAGACATATC
TAAGTATTTAAATTGGTATCAACAAAAACCTGACGGCACGGTAAAATTACTAATATACCATACGTCGCGACTCCAT
TCGGGTGTACCATCGCGTTTTTCGGGATCGGGTTCGGGTACTGATTACTCGCTTACTATATCGAATTTAGAACAAG
AAGATATCGCGACTTATTTTTGCCAACAAGGGAACACGCTACCTTATACCTTTGGTGGAGGGACCAAATTGGAAAT
AACGGGGGGGGGGGGAGTGGAGGAGGAGGTTCAGGGGGGGGGGGTTCAGAAGTTAAATTACAAGAATCAGGACCA
GGATTAGTAGCGCCGTCGCAATCGTTATCAGTTACGTGTACGGTCTCGGGTGTAAGTCTACCGGATTACGGAGTAT
CGTGGATTAGGCAACCCCCGCGCAAGGGGCTTGAATGGCTTGGCGTTATCTGGGGCTCGGAAACTACGTATTATAA
CTCGGCTCTAAAATCACGTTTGACGATAATAAAAGACAATTCGAAATCGCAAGTATTCTTAAAAATGAACAGCCTA
CAAACGGACGACACGGCGATATACTATTGTGCGAAACATTATTATTATGGTGGTAGTTATGCGATGGATTACTGGG
GCCAAGGAACAAGCGTTACCGTATCGTCCACTACGACGCCTGCGCCGCGACCGCCCACGCCGGCGCCTACCATAGC
TTCACAACCATTAAGCCTCCGTCCAGAGGCGTGCCGTCCTGCGGCGGGTGGTGCTGTACATACGAGGGGTCTAGAC
TTTGCGTGTGATATTTATATATGGGCTCCATTAGCGGGGACATGCGGGGTTCTACTACTATCGCTAGTAATAACTC
TATACTGCAAACGTGGACGTAAAAAGTTACTATATATATTTAAACAGCCGTTCATGAGACCGGTACAAACTACGCA
AGAGGAAGATGGGTGCTCCTGTAGATTTCCGGAGGAAGAAGAAGGTGGTTGCGAATTAAGAGTTAAATTCAGTCGA
AGTGCGGACGCGCCAGCGTATCAACAAGGGCAAAATCAACTATACAATGAACTAAATCTTGGTCGAAGAGAAGAAT
ATGACGTATTAGACAAACGGCGTGGACGAGACCCTGAGATGGGTGGAAAACCGCGAAGAAAAAATCCACAAGAAGG
TTTATACAATGAATTACAAAAAGATAAAATGGCGGAGGCATATAGTGAAATAGGTATGAAAGGAGAACGAAGACGT
GGTAAAGGGCATGACGGATTATATCAAGGATTGTCGACGGCAACGAAAGATACTTACGACGCGTTACATATGCAGG
CCCTACCGCCGCGA (SEQ ID NO: 157)
ATGGCCTTACCAGTAACAGCCTTATTACTTCCACTTGCCCTCCTTCTACATGCCGCTAGACCAGATATCCAAATGA
CACAAACGACCTCAAGCCTTTCCGCGTCTCTCGGTGACAGGGTAACTATTAGTTGTCGGGCCTCCCAAGATATTTC
AAAATACTTAAATTGGTATCAACAGAAGCCAGATGGTACCGTGAAGCTCCTCATCTACCACACTAGCCGTCTTCAC
TCTGGAGTACCAAGCAGGTTTTCCGGCTCCGGAAGTGGTACTGACTACAGTTTGACTATAAGCAACCTAGAGCAAG
AGGATATTGCCACATACTTCTGTCAACAAGGAAACACTCTGCCATATACATTCGGTGGTGGTACAAAACTGGAAAT
AACTGGAGGGGGAGGTTCAGGCGGCGGCGGATCTGGGGGGGGTGGCAGTGAGGTCAAATTGCAGGAGTCTGGACCC
GGTCTGGTTGCTCCATCCCAATCACTTTCAGTGACCTGCACTGTATCCGGAGTTTCTCTGCCAGATTATGGAGTGA
GTTGGATCCGCCAGCCTCCACGCAAGGGCCTTGAATGGCTCGGGGTTATATGGGGTAGTGAAACTACATACTATAA
TAGCGCTCTGAAATCTCGACTCACAATCATTAAGGATAATAGCAAAAGCCAGGTGTTTTTGAAGATGAATAGCCTG
CAAACCGATGATACCGCCATATATTACTGTGCCAAACATTACTATTACGGAGGCTCCTACGCTATGGATTATTGGG
GACAGGGGACAAGCGTGACCGTTAGCTCTACTACTACCCCCGCCCCTCGCCCTCCAACTCCAGCTCCCACCATTGC
CAGTCAACCACTATCCTTAAGACCCGAGGCATGCAGACCCGCCGCAGGGGGCGCCGTACATACCCGAGGTTTGGAT
TTTGCCTGCGACATCTATATCTGGGCCCCCCTTGCTGGGACATGTGGAGTGTTGCTACTCTCGTTGGTTATCACTC
TGTATTGCAAGAGAGGAAGAAAGAAACTATTATATATTTTTAAGCAGCCATTCATGAGACCGGTGCAGACTACCCA
AGAAGAAGACGGATGTTCATGTAGGTTCCCTGAAGAAGAGGAAGGAGGCTGCGAACTGCGAGTGAAATTTTCCAGA
TCTGCCGATGCACCAGCTTATCAACAAGGACAAAACCAGCTATATAATGAACTTAATTTAGGGAGACGAGAGGAGT
ACGACGTCCTGGACAAACGCCGCGGAAGAGACCCTGAGATGGGAGGTAAGCCCAGAAGAAAAAACCCACAAGAAGG
ACTGTACAATGAACTCCAAAAAGACAAAATGGCTGAAGCGTATTCCGAAATTGGCATGAAAGGAGAACGGAGAAGA
GGTAAGGGTCACGACGGCTTATATCAGGGACTCTCTACAGCAACCAAAGACACATATGATGCACTACACATGCAGG
CACTACCACCCAGG (SEQ ID NO: 158)
ATGGCTCTACCAGTCACAGCACTACTATTGCCGTTGGCTCTATTGCTTCACGCGGCGCGACCGGATATACAGATGA
CCCAAACGACGTCGAGTCTCTCGGCGTCGTTGGGTGATCGTGTAACGATATCGTGTCGTGCGTCGCAAGATATCTC
GAAATATCTAAATTGGTATCAACAAAAACCGGACGGGACGGTGAAATTATTAATATATCACACGTCCCGTCTACAT
TCTGGTGTTCCGAGTCGATTTAGTGGGAGTGGTTCGGGTACGGACTACTCCCTAACTATATCGAACTTGGAACAAG
AGGATATAGCGACGTACTTCTGCCAACAAGGTAATACGCTACCGTATACCTTTGGCGGAGGTACGAAGCTAGAAAT
AACTGGTGGGGGTGGGAGTGGTGGTGGTGGTTCGGGTGGTGGTGGTTCTGAAGTGAAACTTCAAGAATCGGGACCG
GGGCTGGTAGCCCCGTCCCAATCGTTATCGGTAACGTGCACCGTATCGGGTGTATCGTTACCGGATTATGGTGTAT
CGTGGATACGCCAACCGCCCCGTAAAGGCCTAGAATGGCTAGGTGTTATATGGGGTTCGGAAACGACGTACTACAA
TAGTGCGTTGAAATCGCGACTTACTATTATCAAGGATAATAGTAAGTCGCAAGTATTTTTAAAGATGAATTCGTTA
CAGACGGACGATACCGCGATATACTATTGCGCGAAGCACTATTATTATGGTGGTTCGTATGCGATGGATTATTGGG
GTCAAGGTACGTCTGTAACGGTTTCATCTACTACTACTCCGGCACCACGACCACCAACTCCTGCTCCTACTATAGC
GTCGCAACCGTTATCGTTGCGACCTGAAGCATGTAGACCAGCGGCAGGTGGTGCTGTTCATACGCGAGGTCTAGAC
TTCGCGTGTGATATATATATATGGGCCCCGCTTGCGGGTACGTGTGGTGTACTATTACTATCGTTAGTAATAACGC
TATACTGCAAGCGGGGCCGAAAAAAATTATTATATATATTTAAACAACCGTTTATGCGGCCGGTACAAACGACGCA
AGAGGAAGATGGGTGTTCTTGTCGTTTTCCTGAAGAGGAAGAAGGTGGATGCGAACTACGTGTAAAATTTTCGCGT
AGTGCGGATGCGCCGGCCTATCAACAAGGCCAAAATCAACTCTATAACGAGTTGAATCTAGGACGTCGTGAAGAGT
ACGACGTCCTAGATAAACGACGTGGTCGTGACCCGGAAATGGGTGGCAAACCGCGTCGTAAAAACCCGCAAGAGGG
TTTATATAATGAGCTACAAAAAGATAAAATGGCTGAAGCGTACTCGGAAATTGGTATGAAAGGTGAACGTCGTCGT
GGTAAAGGCCATGACGGTTTATATCAAGGTCTGAGTACGGCGACGAAGGACACCTATGATGCTCTACATATGCAAG
CGTTACCGCCGCGT (SEQ ID NO: 159)
ATGGCTCTACCCGTCACGGCGTTATTACTTCCGCTCGCGTTGCTATTACACGCAGCGCGACCGGATATACAAATGA
CGCAAACGACGTCGTCGTTATCGGCGTCGCTAGGAGATCGTGTGACGATCTCTTGCCGAGCGTCGCAAGATATCTC
GAAATATCTAAATTGGTATCAACAAAAACCGGACGGTACGGTTAAATTGTTGATATATCATACGTCGAGGCTACAT
TCTGGTGTTCCGAGTCGATTTAGTGGGAGTGGTTCGGGTACGGACTACTCCCTAACTATATCGAACTTGGAACAAG
AGGATATAGCCACGTATTTTTGCCAACAGGGCAATACCCTCCCCTACACGTTTGGGGGAGGGACGAAACTAGAAAT
TACAGGTGGTGGTGGTTCTGGGGGTGGTGGATCAGGTGGTGGTGGTTCTGAAGTGAAACTTCAAGAATCAGGACCG
GGTCTAGTCGCCCCCAGTCAATCGCTATCGGTAACCTGTACGGTTTCTGGTGTCAGCTTACCGGATTATGGTGTAA
GCTGGATACGTCAACCGCCCCGTAAAGGCCTAGAATGGCTAGGTGTTATATGGGGTTCGGAGACGACGTACTACAA
TAGTGCGTTAAAATCGCGACTTACTATTATCAAAGATAATAGTAAGTCGCAGGTTTTTTTAAAGATGAACTCTTTA
CAAACGGATGATACGGCGATATACTATTGCGCGAAGCACTATTATTATGGTGGTTCGTATGCGATGGATTACTGGG
GGCAGGGTACGTCGGTAACTGTGAGTTCGACTACGACTCCTGCTCCTAGACCGCCGACTCCGGCGCCAACTATAGC
ATCGCAGCCTCTATCGCTTCGTCCGGAAGCGTGTAGACCAGCTGCAGGTGGTGCTGTACATACGCGAGGTCTAGAC
TTCGCGTGTGATATATATATATGGGCGCCCCTAGCGGGTACGTGTGGGGTGCTATTACTATCGTTAGTAATAACGC
TATATTGCAAACGTGGTAGGAAAAAGCTACTATATATATTTAAACAACCTTTTATGCGTCCGGTACAAACGACGCA
AGAAGAGGATGGTTGTAGTTGTCGATTTCCGGAAGAAGAAGAGGGAGGTTGCGAACTACGAGTAAAATTTAGTCGA
TCGGCGGATGCGCCGGCGTATCAACAAGGTCAAAATCAATTATATAATGAATTAAATCTAGGACGTCGTGAAGAAT
ATGACGTCCTAGATAAACGACGTGGTCGTGACCCAGAAATGGGTGGTAAACCGCGGCGTAAAAACCCGCAAGAAGG
TTTATATAATGAGCTTCAAAAAGATAAAATGGCGGAAGCTTATTCGGAAATAGGTATGAAAGGTGAACGCCGTCGT
GGTAAAGGGCATGACGGTCTTTACCAAGGTTTGTCGACGGCGACGAAAGATACGTATGATGCGTTACACATGCAAG
CGTTACCGCCGCGT (SEQ ID NO: 160)
ATGGCATTACCCGTTACCGCCCTCCTCTTGCCCCTCGCCCTCTTGCTCCACGCCGCCCGGCCAGATATCCAGATGA
CCCAGACCACCTCCTCCCTCAGCGCCTCCCTCGGCGACCGGGTGACCATCTCTTGCCGGGCCTCACAGGACATCAG
CAAGTACCTCAATTGGTACCAGCAGAAGCCCGACGGCACCGTGAAGCTTCTGATCTACCACACCTCCCGGCTGCAC
AGCGGGGTGCCCAGCAGGTTCAGCGGCAGCGGGTCAGGCACCGACTACAGCCTGACCATCTCCAACCTGGAGCAGG
AGGACATCGCCACCTACTTCTGCCAGCAGGGGAACACCCTGCCCTACACCTTCGGCGGGGGCACCAAGCTGGAGAT
CACTGGCGGCGGGGGGAGCGGGGGGGGGGGGTCTGGGGGCGGGGGTAGTGAGGTGAAGCTGCAGGAGAGCGGACCA
GGCCTGGTGGCCCCCAGCCAGAGCCTGTCCGTGACCTGCACCGTGTCTGGCGTGAGCCTGCCAGACTACGGCGTGT
CCTGGATCCGCCAGCCACCCAGGAAGGGCCTGGAGTGGCTGGGCGTGATCTGGGGCAGCGAGACCACCTACTACAA
CAGCGCGCTGAAGTCCAGGCTGACCATCATCAAGGACAACAGCAAGAGCCAGGTGTTCCTGAAGATGAACAGCCTG
CAGACCGACGACACCGCCATCTACTACTGTGCGAAGCACTACTACTATGGCGGGTCCTACGCCATGGATTACTGGG
GCCAGGGGACCTCAGTGACCGTGAGCAGCACCACTACCCCCGCCCCCAGACCCCCCACCCCCGCTCCCACCATCGC
CAGCCAGCCTCTGAGCCTGCGGCCAGAGGCCTGCAGGCCTGCCGCCGGGGGGGCCGTGCACACCCGCGGGCTGGAC
TTCGCCTGCGACATCTACATCTGGGCCCCCCTGGCCGGCACCTGCGGGGTGCTGCTGCTGAGCCTGGTGATCACCC
TGTACTGCAAGCGCGGGAGGAAGAAGCTGCTGTACATCTTCAAGCAGCCCTTCATGAGGCCCGTGCAGACCACCCA
GGAGGAGGATGGCTGCAGCTGCAGGTTCCCTGAGGAGGAGGAGGGCGGCTGCGAGCTGCGGGTGAAGTTCTCCCGC
AGCGCAGACGCCCCCGCCTACCAGCAGGGCCAGAATCAGCTGTACAACGAGCTGAATCTGGGCCGGCGGGAGGAGT
ACGACGTGCTGGACAAGCGCCGGGGCCGGGACCCCGAGATGGGGGGCAAGCCCCGGCGCAAGAATCCTCAGGAGGG
CCTGTACAACGAGCTGCAGAAGGATAAGATGGCCGAGGCCTACAGCGAGATCGGGATGAAGGGCGAGAGGAGGCGC
GGCAAGGGGCACGACGGCCTGTATCAGGGCCTGTCCACCGCCACCAAGGACACCTACGACGCCCTGCACATGCAGG
CCCTGCCCCCCCGG (SEQ ID NO: 161)
ATGGCTCTACCAGTTACAGCCCTCTTGCTCCCTTTGGCCTTGTTGCTCCACGCAGCAAGGCCAGACATCCAGATGA
CTCAGACTACCTCTAGTTTGAGTGCATCCTTGGGAGACCGGGTGACTATCAGTTGCCGGGCCTCCCAGGACATCAG
CAAGTACCTCAATTGGTACCAGCAGAAGCCGGACGGCACCGTGAAGTTGCTGATCTACCACACCAGCAGGCTGCAC
AGCGGCGTGCCCTCCCGGTTCTCCGGGAGCGGGTCAGGGACCGACTACTCCCTGACCATCAGCAACCTGGAGCAGG
AGGACATCGCTACTTACTTCTGCCAGCAGGGGAACACCCTGCCCTACACCTTTGGCGGAGGGACAAAGCTGGAGAT
CACTGGTGGGGGCGGGAGTGGGGGGGGGGGGAGCGGGGGTGGGGGGTCTGAGGTGAAGCTGCAGGAGTCCGGCCCC
GGGCTGGTCGCCCCCTCACAGAGCCTGTCGGTGACATGCACTGTGAGCGGGGTGAGCCTGCCCGACTACGGGGTGA
GCTGGATCCGCCAGCCCCCCCGGAAGGGCCTGGAGTGGCTCGGAGTGATCTGGGGCTCCGAGACCACTTACTACAA
CAGCGCCCTGAAGTCCAGGTTGACTATTATCAAGGATAATAGCAAGTCTCAGGTGTTCCTGAAGATGAACAGCCTG
CAGACCGATGACACCGCCATATACTACTGTGCGAAGCACTACTACTATGGCGGGTCATACGCCATGGACTACTGGG
GCCAGGGCACCTCAGTGACCGTCAGCTCCACCACCACCCCCGCTCCCCGCCCCCCCACTCCCGCCCCCACCATCGC
ATCCCAGCCCCTGTCCCTCCGGCCAGAGGCCTGCCGGCCCGCCGCGGGCGGCGCCGTGCACACCCGCGGGCTGGAC
TTCGCCTGCGACATCTACATCTGGGCTCCTCTGGCCGGCACCTGCGGCGTCCTCCTCCTGAGCCTGGTGATCACTC
TGTACTGCAAGCGCGGAAGGAAGAAGCTGCTGTACATCTTCAAGCAGCCCTTCATGCGCCCAGTGCAGACCACCCA
GGAGGAGGACGGCTGCAGCTGCCGGTTCCCAGAGGAGGAGGAGGGCGGGTGCGAGCTGCGGGTGAAGTTCTCCCGC
AGCGCCGACGCACCCGCCTACCAGCAGGGCCAGAATCAGCTCTACAACGAGCTGAATCTGGGCCGGCGGGAGGAGT
ACGATGTGCTGGACAAGCGGAGGGGCAGGGATCCCGAGATGGGGGGTAAGCCTCGGCGGAAGAATCCTCAGGAGGG
GCTCTACAACGAGCTCCAGAAGGATAAGATGGCCGAGGCTTACTCCGAGATCGGGATGAAGGGGGAGAGGCGCCGG
GGTAAGGGCCACGATGGCCTTTACCAGGGCCTCTCCACCGCAACAAAGGACACCTATGATGCCCTGCACATGCAGG
CCCTGCCCCCCCGG (SEQ ID NO: 162)
ATGGCTTTACCCGTTACAGCCCTGTTGCTGCCCCTTGCCCTGCTGCTGCACGCCGCCCGGCCAGATATCCAGATGA
CGCAGACCACCAGCTCATTGTCAGCCTCCCTGGGGGATCGGGTGACCATCAGCTGCCGGGCCAGTCAGGACATCAG
CAAGTACCTGAACTGGTACCAGCAGAAGCCTGATGGCACCGTGAAGCTGCTGATCTACCACACCAGCAGGCTTCAC
TCCGGCGTGCCTTCCCGCTTCTCTGGGTCCGGCTCCGGCACAGACTACAGTCTGACCATCTCCAACCTGGAGCAGG
AGGACATCGCTACTTACTTCTGCCAGCAGGGGAACACCCTGCCCTACACCTTCGGCGGGGGCACCAAGCTGGAGAT
CACTGGCGGTGGTGGGAGCGGGGGTGGGGGGTCTGGGGGCGGGGGTAGCGAGGTGAAGCTGCAGGAGTCCGGACCC
GGCCTGGTCGCCCCCAGCCAGTCCCTGTCCGTGACATGCACCGTGTCTGGCGTGAGCCTGCCAGACTACGGCGTGT
CATGGATCAGGCAGCCCCCCCGGAAGGGGCTGGAGTGGCTGGGGGTGATCTGGGGGTCCGAGACTACCTACTACAA
CAGCGCGCTGAAGTCCAGGCTGACCATCATCAAGGACAACTCCAAGAGTCAGGTGTTCTTGAAGATGAACAGCCTG
CAGACCGATGACACCGCCATATACTACTGTGCGAAGCACTACTACTATGGCGGGTCATACGCCATGGATTACTGGG
GCCAGGGGACCTCAGTGACCGTGAGCAGCACCACCACCCCCGCCCCCAGACCCCCCACCCCCGCTCCCACCATCGC
CAGCCAGCCCCTGAGCCTGCGCCCCGAGGCCTGCCGGCCCGCGGCCGGCGGCGCCGTGCACACTCGGGGGCTGGAC
TTCGCCTGCGATATCTATATCTGGGCCCCTCTGGCCGGCACCTGCGGCGTCCTCCTCCTGAGTCTGGTGATCACCC
TGTACTGCAAGCGCGGAAGGAAGAAGCTGCTGTACATCTTCAAGCAGCCCTTCATGCGCCCAGTGCAGACCACTCA
GGAGGAGGACGGCTGCAGCTGCCGGTTCCCAGAGGAGGAGGAGGGGGGCTGCGAGCTGCGGGTGAAGTTCTCCCGC
AGCGCAGACGCCCCCGCCTACCAGCAGGGCCAGAATCAGCTCTACAACGAGCTGAATCTGGGCCGGCGGGAGGAGT
ACGATGTCCTGGACAAGCGCCGGGGCCGGGACCCAGAGATGGGCGGGAAGCCACGCCGGAAGAACCCCCAGGAGGG
GCTGTACAATGAGCTGCAGAAGGATAAGATGGCCGAGGCCTATTCCGAGATCGGAATGAAGGGCGAGCGGCGGCGG
GGCAAGGGGCACGACGGGCTGTACCAGGGGCTGAGCACCGCAACAAAGGACACCTATGATGCCCTGCACATGCAGG
CTCTGCCCCCCCGG (SEQ ID NO: 163)
ATGGCTCTGCCTGTGACAGCTCTGCTGCTGCCTCTGGCTCTGCTTCTGCATGCCGCCAGACCTGACATCCAGATGA
CCCAGACAACCAGCAGCCTGTCTGCCAGCCTGGGCGACAGAGTGACCATCAGCTGTAGAGCCAGCCAGGACATCAG
CAAGTACCTGAACTGGTATCAGCAGAAACCCGACGGCACCGTGAAGCTGCTGATCTACCACACCAGCAGACTGCAC
AGCGGCGTGCCATCTAGATTTTCTGGCAGCGGCTCTGGCACCGACTACAGCCTGACAATCAGCAACCTGGAACAAG
AGGATATCGCTACCTACTTCTGCCAGCAAGGCAACACCCTGCCTTACACATTCGGCGGAGGCACCAAGCTGGAAAT
CACAGGTGGCGGAGGATCTGGCGGAGGTGGAAGCGGAGGCGGTGGATCTGAAGTGAAACTGCAAGAGTCTGGCCCT
GGCCTGGTGGCTCCATCTCAGTCTCTGAGCGTGACCTGTACCGTCAGCGGAGTGTCTCTGCCTGATTACGGCGTGT
CCTGGATCAGACAGCCTCCTAGAAAAGGCCTGGAATGGCTGGGCGTGATCTGGGGCAGCGAGACAACCTACTACAA
CAGCGCCCTGAAGTCCAGGCTGACCATCATCAAGGACAACTCCAAGAGCCAGGTGTTCCTGAAGATGAACAGCCTG
CAGACCGACGACACCGCCATCTACTACTGCGCCAAGCACTACTACTACGGCGGCAGCTACGCCATGGATTACTGGG
GCCAGGGAACCAGCGTGACCGTGTCTAGCACAACAACCCCTGCTCCTAGACCTCCTACACCAGCTCCTACAATCGC
CAGCCAGCCTCTGTCTCTGAGGCCAGAGGCTTGTAGACCTGCTGCTGGCGGAGCTGTGCACACAAGAGGCCTGGAT
TTCGCCTGCGACATCTACATCTGGGCCCCTCTGGCTGGAACATGTGGCGTGCTGCTGCTGAGCCTGGTCATCACCC
TGTATTGCAGAGTGAAGTTCAGCAGAAGCGCTGACGCCCCTGCTTACCAGCAGGGACAGAACCAGCTGTTTAACGA
GCTGAACCTGGGGAGAAGAGAAGAGTTCGACGTGCTGGACAAGCGGAGAGGCAGAGATCCTGAGATGGGCGGCAAG
CCCAGAAGAAAGAACCCTCAAGAGGGCCTGTTTAATGAGCTGCAGAAAGACAAGATGGCCGAGGCCTTCAGCGAGA
TCGGAATGAAGGGCGAGCGCAGAAGAGGCAAGGGACACGATGGACTGTTCCAGGGCCTGAGCACAGCCACCAAGGA
CACATTCGACGCCCTGCACATGCAGGCCCTGCCTCCAAGA (SEQ ID NO: 164)
ATGCTGCTGCTGGTGACAAGCCTGCTGCTGTGCGAGCTGCCCCACCCCGCCTTTCTGCTGATCCCCGACATCCAGA
TGACCCAGACCACCAGCAGCCTGAGCGCCAGCCTGGGCGACAGAGTGACCATCAGCTGCCGGGCCAGCCAGGACAT
CAGCAAGTACCTGAACTGGTATCAGCAGAAACCCGACGGCACCGTGAAGCTGCTGATCTACCACACCTCCCGGCTG
CACAGCGGCGTGCCCAGCAGATTTTCTGGCAGCGGCAGCGGCACCGACTACAGCCTGACCATCTCCAACCTGGAAC
AAGAAGATATCGCTACCTATTTTTGTCAACAAGGCAACACCCTGCCCTACACCTTCGGCGGAGGCACCAAACTGGA
AATCACCGGCAGCACCAGCGGCTCCGGCAAGCCTGGATCTGGCGAGGGCAGCACCAAGGGCGAAGTGAAGCTGCAA
GAAAGCGGCCCTGGCCTGGTGGCCCCTAGCCAGAGCCTGTCCGTGACCTGTACCGTGTCCGGCGTGTCCCTGCCCG
ACTACGGCGTGTCCTGGATCCGGCAGCCCCCCAGAAAGGGCCTGGAATGGCTGGGCGTGATCTGGGGCAGCGAGAC
AACCTACTACAACAGCGCCCTGAAGTCCCGGCTGACCATCATCAAGGACAACAGCAAGAGCCAGGTGTTCCTGAAG
ATGAACAGCCTGCAGACCGACGACACCGCCATCTACTACTGCGCCAAGCACTACTACTACGGCGGCAGCTACGCCA
TGGACTACTGGGGCCAGGGCACCAGCGTGACCGTGTCCAGCTTCGTGCCCGTGTTTCTGCCCGCCAAGCCTACCAC
CACCCCTGCCCCTAGACCTCCCACCCCAGCCCCAACAATCGCCAGCCAGCCTCTGTCCCTGCGGCCCGAAGCCTGT
AGACCAGCTGCCGGCGGAGCCGTGCACACCAGAGGCCTGGACTTCGCCTGCGACATCTACATCTGGGCCCCTCTGG
CCGGCACCTGTGGCGTGCTGCTGCTGAGCCTGGTGATCACCCTGTACTGCAACCACCGGAACAGAAGCAAGCGGAG
CCGGCTGCTGCACAGCGACTACATGAACATGACCCCAAGACGGCCTGGCCCCACCCGGAAGCACTACCAGCCTTAC
GCCCCTCCCAGAGACTTCGCCGCCTACCGGTCCAGAGTGAAGTTCAGCAGATCCGCCGACGCCCCTGCCTACCAGC
AGGGACAGAACCAGCTGTACAACGAGCTGAACCTGGGCAGACGGGAAGAGTACGACGTGCTGGACAAGCGGAGAGG
CCGGGACCCCGAGATGGGCGGAAAGCCCAGACGGAAGAACCCCCAGGAAGGCCTGTATAACGAACTGCAGAAAGAC
AAGATGGCCGAGGCCTACAGCGAGATCGGCATGAAGGGCGAGCGGAGGCGCGGCAAGGGCCACGATGGCCTGTACC
AGGGCCTGAGCACCGCCACCAAGGACACCTACGACGCCCTGCACATGCAGGCCCTGCCCCCCAGA (SEQ ID
NO: 165)
ATGGCATTACCAGTTACAGCCCTCCTCCTCCCTCTCGCTCTCCTCCTCCACGCCGCCAGGCCAGATATCCAGATGA
CACAGACCACCAGCAGCCTCAGCGCCAGCCTCGGCGATAGAGTGACAATCTCTTGCAGAGCCTCCCAGGACATCAG
CAAGTACCTCAATTGGTACCAGCAGAAGCCCGACGGCACCGTGAAGCTCCTGATCTACCACACCAGCAGACTGCAC
AGCGGCGTGCCTTCTAGATTCTCTGGAAGCGGCAGCGGCACCGACTACAGCCTGACCATCAGCAACCTGGAGCAGG
AGGACATCGCCACCTACTTCTGCCAGCAGGGCAATACCCTGCCCTACACCTTCGGCGGCGGCACAAAACTGGAAAT
TACAGGTGGTGGTGGTTCTGGGGGAGGAGGGTCTGGTGGTGGTGGTAGTGAAGTGAAACTGCAGGAATCTGGACCA
GGACTGGTGGCCCCATCTCAGTCTCTGAGCGTGACATGTACAGTGTCTGGCGTGTCTCTGCCTGATTACGGAGTGT
CTTGGATTAGACAGCCTCCTCGGAAGGGCCTGGAGTGGCTGGGAGTGATCTGGGGCAGCGAGACCACCTACTACAA
CAGCGCCCTGAAGTCCAGGCTGACCATCATCAAGGACAACAGCAAGTCCCAGGTGTTCCTGAAGATGAACAGCCTG
CAGACCGACGACACCGCCATCTACTACTGCGCCAAGCACTACTACTACGGCGGCAGCTACGCCATGGATTACTGGG
GCCAGGGCACCAGCGTGACGGTGTCTTCTACAACAACACCTGCTCCTAGGCCTCCTACACCAGCTCCTACAATTGC
TTCTCAGCCGCTGTCTCTGCGGCCAGAAGCTTGTAGGCCTGCTGCAGGAGGAGCGGTGCATACAAGAGGACTGGAC
TTTGCCTGTGATATCTACATCTGGGCCCCTCTGGCCGGCACCTGCGGCGTGCTGCTGCTGAGCCTGGTGATCACCC
TGTACTGCAACCACAGAAACAGAAGCAAGAGAAGCAGGCTGCTGCACAGCGACTACATGAATATGACACCTAGGAG
ACCTGGCCCTACAAGAAAACATTATCAGCCTTATGCCCCTCCTAGAGATTTTGCCGCCTATAGAAGCCGGGTGAAG
TTTTCTAGATCTGCTGATGCCCCAGCCTATCAGCAGGGCCAGAACCAGTTATATAATGAACTGAACCTGGGCCGGC
GGGAGGAGTACGATGTGCTGGATAAGAGAAGAGGCAGAGATCCTGAAATGGGAGGAAAACCTAGAAGAAAGAATCC
CCAGGAGGGCCTATATAACGAACTGCAGAAGGACAAGATGGCCGAGGCCTACAGCGAGATCGGCATGAAGGGCGAG
AGAAGAAGAGGCAAGGGCCACGATGGACTGTATCAGGGGCTGAGCACCGCCACCAAGGATACCTACGATGCCCTGC
ACATGCAGGCCCTGCCTCCTAGA (SEQ ID NO: 166)
ATGCTATTATTAGTCACCAGCCTCCTCCTTTGCGAGCTCCCCCACCCCGCCTTCCTCCTCATCCCCGACATCCAGA
TGACCCAGACCACCAGCAGCCTCAGCGCCAGCCTCGGCGATAGAGTGACAATCTCTTGCAGAGCCTCCCAGGACAT
CAGCAAGTACCTCAATTGGTACCAGCAGAAGCCCGACGGCACCGTGAAGCTGCTGATCTACCACACCAGCAGACTG
CACAGCGGCGTGCCTTCTAGATTCTCTGGAAGCGGCAGCGGCACCGACTACAGCCTGACCATCAGCAACCTGGAGC
AGGAGGACATCGCCACCTACTTCTGCCAGCAGGGCAATACCTTGCCCTACACCTTCGGCGGCGGCACAAAGCTGGA
AATTACAGGTGGTGGTGGTAGTGGTGGTGGTGGTTCTGGTGGTGGTGGATCTGAAGTGAAACTGCAGGAATCTGGA
CCTGGCCTGGTGGCCCCTTCTCAGTCGCTGAGCGTGACATGTACAGTGAGCGGCGTGAGCCTGCCTGATTATGGAG
TGAGCTGGATCAGACAGCCTCCAAGAAAGGGCCTGGAGTGGCTGGGCGTGATCTGGGGCTCCGAGACCACCTACTA
CAACAGCGCCCTGAAGTCCAGGCTGACCATCATCAAGGACAACAGCAAGAGCCAGGTGTTCCTGAAGATGAACAGC
CTGCAGACCGATGACACCGCCATCTACTACTGCGCCAAGCACTACTACTACGGCGGCAGCTACGCCATGGATTACT
GGGGCCAGGGCACATCTGTGACAGTGTCTTCTGCTGCTGCTCCAGCTCCTAGACCTCCTACACCTGCTCCTACAAT
TGCTTCTCAGCCGCTGAGCCTGAGACCTGAAGCTTGTAGACCTGCTGCTGGAGGAGCTGTGCATACAAGAGGACTG
GATTTTGATACCGCCCTGGCCGCCGTGATCTGTTCTGCCCTGGCCACAGTGCTGCTGGCCCTGCTGATCCTGTGCG
TGATCTACTGCAAGAGACAGCCCAGAAGAAAGAAGCTGCTGTACATCTTCAAGCAGCCCTTCATGAGACCTGTGCA
GACAACACAGGAGGAGGATGGCTGTTCCTGTAGATTTCCAGAGGAAGAGGAGGGCGGCTGCGAGCTGAGAGTGAAA
TTTTCTAGATCTGCCGATGCCCCTGCCTACCAGCAGGGCCAGAACCAGCTATATAATGAACTGAACCTGGGCAGAA
GAGAGGAGTACGACGTGCTGGATAAGAGAAGAGGCAGAGATCCTGAAATGGGAGGAAAACCTAGAAGAAAGAATCC
GCAGGAGGGCCTATATAACGAACTGCAGAAGGACAAGATGGCCGAGGCCTACAGCGAGATCGGCATGAAGGGCGAG
AGAAGAAGAGGCAAGGGCCACGATGGACTGTATCAGGGGCTGAGCACAGCCACCAAGGATACCTACGATGCCCTGC
ACATGCAGGCCCTGCCTCCTAGA (SEQ ID NO: 167)
ATGGCTCTACCCGTTACAGCATTACTACTACCTCTTGCCCTCCTCCTCCACGCCGCCAGACCAGATATCCAGATGA
CCCAGACCACCTCTAGCCTCAGCGCCAGCCTCGGCGATAGAGTGACAATCTCTTGCAGAGCCTCCCAGGACATCAG
CAAGTACCTCAATTGGTACCAGCAGAAGCCCGACGGCACCGTGAAGCTCCTGATCTACCACACCAGCAGGCTGCAC
TCTGGCGTGCCATCTAGATTTTCTGGAAGCGGCAGCGGCACAGATTACAGCCTGACCATCAGCAATCTGGAGCAGG
AGGACATCGCCACCTACTTCTGCCAGCAGGGCAATACCCTGCCCTACACCTTCGGCGGCGGCACAAAACTGGAAAT
TACAGGTGGTGGTGGTTCAGGGGGAGGAGGGTCTGGTGGTGGAGGTTCTGAAGTGAAACTGCAGGAATCTGGACCT
GGCCTGGTGGCCCCTTCTCAGTCGCTGAGCGTGACATGTACCGTGAGCGGCGTGAGCCTGCCTGATTACGGGGTGA
GCTGGATCAGACAGCCCCCTAGAAAGGGCCTGGAATGGCTGGGCGTGATCTGGGGCAGCGAGACCACCTACTACAA
CAGCGCCCTGAAGTCCAGGCTGACCATCATCAAGGACAACAGCAAGTCCCAGGTGTTCCTGAAGATGAACAGCCTG
CAGACCGACGACACCGCCATCTACTACTGCGCCAAGCACTACTACTACGGCGGCAGCTACGCCATGGATTACTGGG
GCCAGGGCACCTCTGTGACAGTGAGCAGCGCCGCTGCCATTGAGGTGATGTACCCTCCCCCCTACCTGGACAACGA
GAAGTCCAACGGCACCATCATCCACGTGAAGGGCAAGCACCTGTGCCCTAGCCCCCTGTTTCCAGGCCCATCTAAG
CCTTTTTGGGTGCTGGTGGTGGTGGGCGGCGTGCTGGCCTGCTACAGCCTGCTGGTGACCGTGGCCTTCATCATCT
TCTGGGTGAGAAGCAAGAGGTCCCGGCTGCTGCACAGCGACTACATGAATATGACACCCAGGAGACCTGGCCCTAC
AAGAAAACATTATCAGCCTTATGCCCCTCCTAGAGATTTTGCCGCCTATAGAAGCCGGGTGAAGTTTTCTAGAAGC
GCTGATGCCCCAGCCTACCAGCAGGGCCAGAACCAGCTGTACAACGAGCTGAACCTGGGCAGAAGAGAGGAGTACG
ACGTGCTGGATAAGAGAAGAGGCAGAGATCCGGAAATGGGAGGAAAGCCTAGAAGAAAGAATCCTCAGGAGGGCCT
GTACAATGAGCTGCAGAAGGACAAGATGGCCGAGGCCTACTCTGAGATCGGCATGAAGGGCGAGAGAAGAAGAGGC
AAGGGCCACGATGGCCTGTACCAGGGCCTGAGCACGGCCACCAAGGATACCTACGATGCCCTGCACATGCAGGCCC
TGCCTCCTAGA (SEQ ID NO: 168)
ATGGCCTTACCAGTGACCGCCTTGCTCCTGCCGCTGGCCTTGCTGCTCCACGCCGCCAGGCCGGACATCCAGATGA
CACAGACTACATCCTCCCTGTCTGCCTCTCTGGGAGACAGAGTCACCATCAGTTGCAGGGCAAGTCAGGACATTAG
TAAATATTTAAATTGGTATCAGCAGAAACCAGATGGAACTGTTAAACTCCTGATCTACCATACATCAAGATTACAC
TCAGGAGTCCCATCAAGGTTCAGTGGCAGTGGGTCTGGAACAGATTATTCTCTCACCATTAGCAACCTGGAGCAAG
AAGATATTGCCACTTACTTTTGCCAACAGGGTAATACGCTTCCGTACACGTTCGGAGGGGGGACCAAGCTGGAGAT
CACAGGTGGCGGTGGCTCGGGCGGTGGTGGGTCGGGTGGCGGCGGATCTGAGGTGAAACTGCAGGAGTCAGGACCT
GGCCTGGTGGCGCCCTCACAGAGCCTGTCCGTCACATGCACTGTCTCAGGGGTCTCATTACCCGACTATGGTGTAA
GCTGGATTCGCCAGCCTCCACGAAAGGGTCTGGAGTGGCTGGGAGTAATATGGGGTAGTGAAACCACATACTATAA
TTCAGCTCTCAAATCCAGACTGACCATCATCAAGGACAACTCCAAGAGCCAAGTTTTCTTAAAAATGAACAGTCTG
CAAACTGATGACACAGCCATTTACTACTGTGCCAAACATTATTACTACGGTGGTAGCTATGCTATGGACTACTGGG
GCCAAGGAACCTCAGTCACCGTCTCCTCAACCACGACGCCAGCGCCGCGACCACCAACACCGGCGCCCACCATCGC
GTCGCAGCCCCTGTCCCTGCGCCCAGAGGCGTGCCGGCCAGCGGGGGGGGCGCAGTGCACACGAGGGGGCTGGAC
TTCGCCTGTGATATCTACATCTGGGCGCCCTTGGCCGGGACTTGTGGGGTCCTTCTCCTGTCACTGGTTATCACCC
TTTACTGCAAACGGGGCAGAAAGAAACTCCTGTATATATTCAAACAACCATTTATGAGACCAGTACAAACTACTCA
AGAGGAAGATGGCTGTAGCTGCCGATTTCCAGAAGAAGAAGAAGGAGGATGTGAACTGAGAGTGAAGTTCAGCAGG
AGCGCAGACGCCCCCGCGTACAAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGAGAAGGAAGAACCCTCAGGAAGG
CCTGTACAATGAACTGCAGAAAGATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACCTACGACGCCCTTCACATGCAGG
CCCTGCCCCCTCGCTAA (SEQ ID NO: 346)
*Because the sequences are RNA sequences, it is understood that T's represent uracil in the sequences of this table.

In some embodiments, the circRNA molecule comprises one or more modified nucleotides. In some embodiments, the circRNA molecule is unmodified.

In some embodiments, the circRNA molecules of the present invention may include one, two, three, or more modifications. In some embodiments, the modified nucleotides are located in coding region(s). In some embodiments, the modified nucleotides are in the untranslated region(s).

In some embodiments, the modifications stabilize the circRNA molecule and enhance resistance to degradation as compared to unmodified nucleotides. In some embodiments, modified nucleotides enhance biological functions of nucleic acid molecules, for example, increase binding to a RNA binding protein or increasing translation.

In some embodiments, the modified nucleotide is one or more of N1-methylpseudouridine, 5-methoxyuridine, N6-methyladenosine, pseudouridine or 5-methylcytosine.

In some embodiments, the modified nucleotide is N1-methylpseudouridine. In some embodiments, the modified nucleotide is 5-methoxyuridine. In some embodiments, the modified nucleotide is N6-methyladenosine. In some embodiments, the modified nucleotide is pseudouridine. In some embodiments, the modified nucleotide is 5-methylcytosine.

In some embodiments, the encoded protein is an anti-CD19 antibody or antigen binding domain. In some embodiments, the encoded protein is any one of SEQ ID Nos: 169-174 selected from Table 5. In some embodiments, the circRNA encodes an amino acid sequence having at least 75% identity to any one of SEQ ID NOs: 169-174. In some embodiments, the circRNA encodes an amino acid sequence having at least 80% identity to any one of SEQ ID NOs: 169-174. In some embodiments, the circRNA encodes an amino acid sequence having at least 85% identity to any one of SEQ ID NOs: 169-174. In some embodiments, the circRNA encodes an amino acid sequence having at least 90% identity to any one of SEQ ID NOs: 169-174. In some embodiments, the circRNA encodes an amino acid sequence having at least 95% identity to any one of SEQ ID NOs: 169-174. In some embodiments, the circRNA encodes an amino acid sequence having at least 100% identity to any one of SEQ ID NOs: 169-174.

TABLE 5
Exemplary Encoded Anti-CD19 Antibody or Antigen Binding Domain
(Amino Acid Sequences)

Encoded Anti-CD19 Amino Acid Sequences
MALPVTALLLPLALLLHAARPDIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR (SEQ ID NO: 169)
MALPVTALLLPLALLLHAARPDIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCRVKFSRSADAPAYQQGQNQLFNELNLGRREEFDVLDKRRGRDPEMGGK
PRRKNPQEGLFNELQKDKMAEAFSEIGMKGERRRGKGHDGLFQGLSTATKDTFDALHMQALPPR (SEQ ID NO:
170)
MLLLVTSLLLCELPHPAFLLIPDIQMTQTTSSLSASLGDRVTISCRASQDISKYLNWYQQKPDGTVKLLIYHTSRL
HSGVPSRFSGSGSGTDYSLTISNLEQEDIATYFCQQGNTLPYTFGGGTKLEITGSTSGSGKPGSGEGSTKGEVKLQ
ESGPGLVAPSQSLSVTCTVSGVSLPDYGVSWIRQPPRKGLEWLGVIWGSETTYYNSALKSRLTIIKDNSKSQVFLK
MNSLQTDDTAIYYCAKHYYYGGSYAMDYWGQGTSVTVSSFVPVFLPAKPTTTPAPRPPTPAPTIASQPLSLRPEAC
RPAAGGAVHTRGLDFACDIYIWAPLAGTCGVLLLSLVITLYCNHRNRSKRSRLLHSDYMNMTPRRPGPTRKHYQPY
APPRDFAAYRSRVKFSRSADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR (SEQ ID NO: 171)
MALPVTALLLPLALLLHAARPDIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCNHRNRSKRSRLLHSDYMNMTPRRPGPTRKHYQPYAPPRDFAAYRSRVK
FSRSADAPAYQQGQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR (SEQ ID NO: 172)
MLLLVTSLLLCELPHPAFLLIPDIQMTQTTSSLSASLGDRVTISCRASQDISKYLNWYQQKPDGTVKLLIYHTSRL
HSGVPSRFSGSGSGTDYSLTISNLEQEDIATYFCQQGNTLPYTFGGGTKLEITGGGGSGGGGSGGGGSEVKLQESG
PGLVAPSQSLSVTCTVSGVSLPDYGVSWIRQPPRKGLEWLGVIWGSETTYYNSALKSRLTIIKDNSKSQVFLKMNS
LQTDDTAIYYCAKHYYYGGSYAMDYWGQGTSVTVSSAAAPAPRPPTPAPTIASQPLSLRPEACRPAAGGAVHTRGL
DFDTALAAVICSALATVLLALLILCVIYCKRQPRRKKLLYIFKQPFMRPVQTTQEEDGCSCRFPEEEEGGCELRVK
FSRSADAPAYQQGQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR (SEQ ID NO: 173)
MALPVTALLLPLALLLHAARPDIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSAAAIEVMYPPPYLDNEKSNGTIIHVKGKHLCPSPLFPGPSK
PFWVLVVVGGVLACYSLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKDKMAEAYSEIGMKGERRRG
KGHDGLYQGLSTATKDTYDALHMQALPPR (SEQ ID NO: 174)
MALPVTALLLPLALLLHAARPDIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDGCSCRFPEEEEGGCELRVKFSR
SADAPAYKQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR (SEQ ID NO: 347)

In some aspects, provided herein is a full-length circRNA sequence selected from Table 6. As discussed here, the circRNA sequences comprise features, including, for example, the 5′ UTR, ribosome recruiting element (such as IRES), coding sequence, and 3′ UTR. In some aspects, provided herein is a full-length circRNA comprising a sequence selected from any one of SEQ ID NOs: 175-233 in Table 6 or SEQ ID NOs: 264 or 269 in Table 7B. In some aspects, provided herein is a circRNA comprising a sequence having at least 80% identity to any one of SEQ ID NOs: 175-233, 264 or 269. In some embodiments, provided herein is a circRNA comprising a sequence having at least 85% identity to any one of SEQ ID NOs: 175-233, 264 or 269. In some embodiments, provided herein is a circRNA comprising a sequence having at least 90% identity to any one of SEQ ID NOs: 175-233, 264 or 269. In some embodiments, provided herein is a circRNA comprising a sequence having at least 95% identity to any one of SEQ ID NOs: 175-233, 264 or 269. In some embodiments, provided herein is a circRNA comprising a sequence having at least 100% identity to any one of SEQ ID NOs: 175-233, 264 or 269.

TABLE 6
Exemplary Full-length CircRNA Sequences*

AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCTAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAAGAGAGGCCGGAAGAAGCTGCTCTACATCTTCAAGCAGCCCTTC
ATGAGGCCCGTGCAGACCACACAAGAAGAGGACGGCTGCTCCTGCAGATTCCCCGAGGAAGAGGAAGGCGGCTGCG
AGCTGAGAGTGAAGTTCAGCAGAAGCGCTGACGCCCCTGCTTACCAGCAGGGACAGAACCAGCTGTACAACGAGCT
GAACCTGGGGAGAAGAGAAGAGTACGACGTGCTGGACAAGCGGAGAGGCAGAGATCCTGAGATGGGCGGCAAGCCC
AGAAGAAAGAACCCTCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTACCAGGGCCTGAGCACAGCCACCAAGGACAC
ATATGACGCCCTGCACATGCAGGCCCTGCCTCCAAGATAATAGAAAAACAAAAAACAAAAACCACACAAATGGTCG
CCGAAACTACTGAAAGCAT (SEQ ID NO: 175)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCACTACCCGTTACAGCATTACTATTACCTCTCGCCCTCCTCCTCCACGC
CGCCAGACCAGATATCCAGATGACACAGACCACCTCTAGCCTCAGCGCCAGCCTCGGCGATAGAGTGACAATCTCT
TGCAGAGCCTCCCAGGACATCAGCAAGTACCTCAATTGGTACCAGCAGAAGCCCGACGGCACCGTGAAGCTCCTGA
TCTACCACACCAGCAGGCTGCACAGCGGCGTGCCATCTAGATTCTCTGGATCTGGATCTGGCACCGATTACAGCCT
GACCATCAGCAATCTGGAGCAGGAGGACATCGCCACCTACTTCTGCCAGCAGGGGAATACCCTGCCCTACACCTTT
GGCGGCGGCACAAAGCTGGAGATTACAGGTGGTGGTGGTTCTGGGGGAGGAGGGTCTGGTGGTGGTGGTAGTGAAG
TGAAACTGCAGGAATCTGGACCTGGCCTGGTGGCCCCTTCTCAGTCGCTGAGCGTGACATGCACCGTGTCTGGCGT
GAGCCTGCCGGATTACGGCGTGAGCTGGATTCGGCAGCCTCCTCGGAAGGGCCTGGAATGGCTGGGCGTGATCTGG
GGCTCCGAGACCACCTACTACAACAGCGCCCTGAAGTCCAGGCTGACCATCATCAAGGACAACAGCAAGTCCCAGG
TGTTCCTGAAGATGAACAGCCTGCAGACCGATGACACGGCCATCTACTACTGCGCCAAGCACTACTACTACGGAGG
CTCCTACGCCATGGATTACTGGGGCCAGGGAACCTCCGTGACAGTGTCTTCTACAACAACACCAGCTCCTAGGCCT
CCTACACCTGCTCCTACAATTGCTTCTCAGCCTCTGTCTCTGAGACCAGAAGCTTGTAGACCAGCTGCTGGAGGAG
CTGTGCATACAAGAGGCCTGGATTTCGCCTGTGACATTTACATCTGGGCTCCACTGGCCGGCACCTGTGGCGTGCT
GCTGCTGAGCCTGGTGATCACCCTGTACTGCAAGAGGGGCAGAAAGAAGCTGCTGTACATCTTCAAGCAGCCTTTC
ATGCGCCCCGTGCAGACAACACAGGAGGAAGATGGCTGCAGCTGTAGATTTCCTGAAGAAGAGGAGGGCGGCTGCG
AGCTGAGAGTGAAATTTTCTAGAAGCGCCGACGCCCCTGCCTACCAGCAGGGCCAGAATCAGTTATATAACGAACT
GAATCTGGGCCGGCGGGAGGAGTACGACGTGCTGGATAAGAGAAGAGGCAGAGATCCTGAAATGGGAGGAAAACCT
AGAAGAAAGAATCCGCAGGAGGGCTTATATAATGAACTGCAGAAGGACAAGATGGCGGAGGCCTACAGCGAGATCG
GCATGAAGGGCGAGAGAAGAAGAGGCAAGGGCCACGATGGCCTGTACCAGGGCCTGAGCACGGCCACCAAGGATAC
CTACGATGCCCTGCACATGCAGGCCCTGCCTCCTAGATAATAGAAAAACAAAAAACAAAAACCACACAAATGGTCG
CCGAAACTACTGAAAGCAT (SEQ ID NO: 176)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCAGTTACAGCCCTCCTTCTTCCTCTCGCCCTCTTGCTCCACGC
CGCCAGGCCAGATATCCAGATGACACAGACCACCAGCAGCCTCAGCGCCAGCCTCGGCGATAGAGTGACAATCTCT
TGCAGAGCCTCCCAGGACATCAGCAAGTACCTCAATTGGTACCAGCAGAAGCCCGACGGCACCGTGAAGTTGCTGA
TCTACCACACCAGCAGACTGCACAGCGGAGTGCCTTCTAGATTCTCTGGAAGCGGCTCCGGCACAGACTACTCGCT
GACCATCAGCAACCTGGAGCAGGAGGACATCGCCACCTACTTCTGCCAGCAGGGCAATACCCTGCCCTACACCTTC
GGCGGCGGCACAAAACTGGAGATTACAGGTGGTGGTGGTAGTGGTGGTGGGGGAAGTGGTGGTGGTGGTTCTGAAG
TGAAACTGCAGGAATCAGGGCCTGGCCTGGTGGCCCCTTCTCAGTCGCTGAGCGTGACCTGTACCGTGTCAGGCGT
GAGCCTGCCAGATTACGGAGTGAGCTGGATTAGACAGCCCCCTCGGAAGGGCCTGGAATGGCTGGGCGTGATCTGG
GGCAGCGAGACCACCTACTACAACAGCGCCCTGAAGTCCAGGCTGACCATCATCAAGGACAACAGCAAGTCCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGCACCTCTGTGACGGTGTCTTCTACAACAACACCTGCTCCTAGGCCT
CCTACACCAGCTCCTACAATTGCTTCTCAGCCGCTGAGTCTGCGGCCAGAAGCTTGTAGGCCTGCTGCAGGAGGAG
CTGTGCATACAAGAGGACTGGACTTTGCCTGTGATATCTACATCTGGGCCCCTCTGGCCGGCACCTGCGGCGTGCT
GCTGCTGAGCCTGGTGATCACCCTGTACTGCAAGCGCGGCCGGAAGAAGCTGCTGTACATCTTCAAGCAGCCCTTC
ATGAGGCCCGTGCAGACAACACAGGAGGAGGATGGCTGCTCCTGTAGATTTCCTGAAGAGGAGGAGGGCGGCTGCG
AGCTGAGAGTGAAATTTTCTAGAAGCGCCGATGCCCCTGCCTACCAGCAGGGCCAGAACCAGTTATATAACGAACT
GAACCTGGGCAGAAGAGAGGAGTACGACGTGCTGGATAAGAGAAGAGGCAGAGATCCAGAAATGGGAGGAAAGCCT
AGAAGAAAGAATCCGCAGGAGGGCCTATATAATGAACTGCAGAAGGACAAGATGGCCGAGGCCTACAGCGAGATCG
GCATGAAGGGCGAGAGAAGAAGAGGCAAGGGCCACGATGGACTGTATCAGGGGCTGAGCACCGCCACCAAGGATAC
CTACGATGCCCTGCACATGCAGGCGCTGCCTCCTAGATAATAGAAAAACAAAAAACAAAAACCACACAAATGGTCG
CCGAAACTACTGAAAGCAT (SEQ ID NO: 177)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCAGTTACAGCCCTCCTCCTCCCTCTCGCCCTCCTTCTCCACGC
CGCCAGACCAGATATCCAGATGACCCAGACCACCTCTAGCCTCAGCGCCAGCCTCGGCGATAGAGTGACAATCTCT
TGCAGAGCCTCCCAGGACATCAGCAAGTACCTCAATTGGTACCAGCAGAAGCCCGACGGCACCGTGAAGCTCCTGA
TCTACCACACCAGCAGGCTGCACTCTGGCGTGCCATCTAGATTTTCTGGATCTGGCAGCGGCACCGACTACAGCCT
GACCATCAGCAACCTGGAGCAGGAGGACATCGCCACCTACTTCTGCCAGCAGGGGAATACCCTGCCCTACACCTTT
GGCGGCGGCACAAAGCTGGAGATTACAGGTGGTGGTGGTTCTGGTGGTGGTGGTAGTGGTGGTGGGGGATCTGAAG
TGAAACTGCAGGAATCTGGACCTGGCCTGGTGGCCCCTTCTCAGTCTCTGAGCGTGACATGCACCGTGTCCGGCGT
GAGCCTGCCGGATTACGGCGTGAGCTGGATTCGGCAGCCTCCTAGAAAGGGCCTGGAATGGCTGGGCGTGATCTGG
GGCAGCGAGACCACCTACTACAACAGCGCGCTGAAGTCCAGGCTGACCATCATCAAGGACAACAGCAAGTCCCAGG
TGTTCCTGAAGATGAACAGCCTGCAGACCGATGACACCGCCATCTACTACTGTGCGAAGCACTACTACTACGGCGG
CTCCTACGCCATGGATTACTGGGGCCAGGGCACCAGCGTGACGGTGTCTTCTACAACAACACCTGCTCCTAGACCT
CCTACACCAGCTCCTACAATTGCTTCTCAGCCTCTGTCGCTGAGACCTGAAGCATGTAGACCTGCTGCTGGAGGAG
CTGTGCATACAAGAGGACTGGATTTTGCCTGCGACATCTACATCTGGGCCCCGCTGGCCGGCACCTGCGGCGTGCT
GCTGCTGAGCCTGGTGATCACCCTGTACTGCAAGAGGGGCAGGAAGAAGCTGCTGTACATCTTCAAGCAGCCCTTC
ATGAGGCCTGTGCAGACCACCCAGGAGGAGGATGGCTGCAGCTGCAGATTTCCTGAAGAAGAGGAGGGAGGCTGCG
AGCTGAGAGTGAAATTTTCCAGGAGCGCCGACGCTCCTGCCTACCAGCAGGGCCAGAATCAGTTATATAATGAACT
GAACCTGGGCCGGCGGGAGGAGTACGACGTGCTGGATAAGAGAAGAGGCAGAGATCCTGAAATGGGAGGAAAGCCT
AGAAGAAAGAATCCGCAGGAGGGCCTATATAACGAACTGCAGAAGGACAAGATGGCCGAGGCCTATTCCGAGATCG
GAATGAAGGGCGAGAGAAGAAGAGGCAAGGGCCACGATGGCCTGTACCAGGGCCTGAGCACGGCCACCAAGGATAC
CTACGACGCCCTGCACATGCAGGCCCTGCCTCCTAGATAATAGAAAAACAAAAAACAAAAACCACACAAATGGTCG
CCGAAACTACTGAAAGCAT (SEQ ID NO: 178)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTACCCGTTACAGCTCTACTGCTGCCGCTGGCACTCTTGCTGCACGC
AGCCAGACCTGACATTCAGATGACGCAGACTACGTCGTCCCTGTCAGCGTCCCTTGGGGATCGGGTTACTATAAGT
TGCCGCGCCAGCCAGGATATAAGTAAATACCTCAATTGGTATCAGCAAAAGCCGGATGGTACCGTGAAGCTCCTCA
TCTACCATACGTCCCGGCTCCACTCTGGTGTGCCGAGCAGGTTCTCCGGGAGTGGGAGTGGGACAGACTACAGCCT
CACGATCAGCAATCTAGAGCAGGAGGACATCGCTACTTACTTTTGCCAGCAGGGGAATACCCTTCCCTACACTTTC
GGTGGCGGCACGAAGTTGGAGATAACTGGAGGCGGCGGGTCTGGCGGCGGTGGGTCAGGCGGTGGGGGCTCGGAGG
TGAAGCTGCAAGAAAGCGGTCCAGGGCTGGTGGCCCCTTCACAGTCCCTTAGTGTGACCTGCACTGTCAGCGGAGT
CTCACTGCCCGATTACGGGGTGAGCTGGATCAGGCAACCGCCGAGGAAGGGGCTGGAATGGTTAGGTGTCATCTGG
GGGTCAGAGACTACTTACTATAACAGCGCTTTGAAGTCTAGACTGACTATCATTAAGGATAACTCCAAATCCCAAG
TCTTCCTCAAGATGAACAGTTTACAGACAGATGACACAGCCATCTACTACTGTGCCAAGCACTACTACTACGGAGG
CTCATATGCGATGGACTATTGGGGCCAAGGTACGTCGGTTACAGTAAGCTCGACGACCACTCCCGCACCTCGGCCT
CCCACCCCTGCTCCTACCATCGCCAGTCAACCCCTGTCCCTGCGGCCTGAGGCCTGTCGTCCCGCGGCCGGGGGTG
CGGTCCATACGCGCGGACTTGACTTTGCGTGTGATATCTACATTTGGGCCCCCTTAGCTGGCACATGTGGTGTTCT
CCTGCTCTCCCTGGTCATCACCTTGTACTGCAAGCGTGGTCGCAAGAAGTTGCTATATATATTTAAGCAGCCCTTC
ATGCGGCCAGTCCAGACTACTCAGGAAGAAGATGGCTGTAGTTGCCGGTTCCCCGAGGAAGAGGAAGGCGGCTGCG
AGCTGCGGGTTAAGTTCTCTCGCAGTGCCGATGCCCCTGCGTATCAGCAGGGGCAGAATCAACTGTATAACGAGTT
GAATCTTGGCCGTCGGGAAGAGTACGATGTTCTCGATAAACGGCGGGGTCGAGATCCCGAGATGGGAGGGAAGCCC
CGGAGAAAGAACCCACAAGAGGGGCTTTATAACGAGCTGCAGAAAGACAAGATGGCCGAGGCCTACTCGGAGATCG
GGATGAAGGGAGAACGGCGCCGGGGTAAGGGCCACGACGGCCTTTACCAAGGGTTAAGTACCGCGACAAAAGATAC
TTATGACGCACTGCACATGCAGGCGTTGCCGCCAAGGTAATAGAAAAACAAAAAACAAAAACCACACAAATGGTCG
CCGAAACTACTGAAAGCAT (SEQ ID NO: 179)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCTTGCCGGTCACTGCATTGCTGCTGCCTCTCGCACTGCTGCTTCATGC
CGCAAGGCCTGACATTCAGATGACTCAGACAACCAGCTCGCTCAGCGCATCGCTGGGGGATCGCGTGACGATCAGT
TGCCGTGCATCCCAGGATATTTCCAAGTACCTCAACTGGTACCAGCAGAAGCCAGATGGTACAGTAAAATTGCTGA
TATACCATACATCCAGATTACACTCAGGCGTACCCTCGCGCTTTAGTGGCTCCGGAAGTGGCACCGATTACTCTTT
GACCATCAGTAATCTCGAGCAAGAAGACATTGCAACGTACTTCTGCCAACAGGGGAACACTCTCCCATACACCTTT
GGGGGAGGTACTAAGCTCGAGATTACGGGTGGCGGGGGGTCGGGCGGTGGCGGCTCTGGAGGCGGAGGGAGCGAGG
TTAAACTGCAGGAGTCAGGCCCTGGGCTTGTAGCTCCCAGTCAGTCACTGTCTGTGACTTGTACTGTGTCAGGTGT
TTCCTTGCCAGACTATGGGGTATCCTGGATCCGCCAGCCACCCCGTAAAGGTCTGGAGTGGCTCGGGGTCATATGG
GGCTCCGAAACTACTTATTACAACTCGGCATTGAAGAGCAGGCTGACAATTATTAAAGATAATAGTAAGTCGCAGG
TGTTCCTTAAAATGAACTCCCTGCAAACTGATGATACGGCCATATACTATTGTGCCAAACACTATTATTACGGCGG
CAGCTATGCTATGGACTATTGGGGTCAGGGCACATCTGTTACCGTTAGTTCGACGACAACGCCCGCACCGCGCCCT
CCAACCCCAGCTCCGACGATTGCATCGCAGCCCCTCTCACTGCGCCCCGAGGCCTGTCGGCCAGCCGCAGGTGGCG
CTGTGCATACACGGGGGCTGGACTTCGCATGCGATATTTATATCTGGGCTCCACTGGCCGGGACGTGCGGAGTTCT
TCTCCTGTCACTGGTGATCACCCTGTATTGTAAGCGCGGGCGGAAGAAGCTGCTATATATTTTTAAGCAGCCCTTT
ATGAGGCCTGTGCAGACGACACAGGAAGAAGACGGGTGCAGTTGCCGATTCCCTGAGGAAGAGGAGGGGGGCTGCG
AGCTGCGCGTGAAATTTTCGCGCTCCGCTGACGCACCTGCGTACCAGCAAGGCCAGAATCAACTCTACAATGAGTT
GAATTTGGGGAGACGCGAAGAGTACGACGTCCTCGACAAGCGACGAGGGAGGGATCCTGAAATGGGAGGAAAGCCT
CGCAGAAAGAACCCTCAGGAGGGGTTGTACAATGAGTTACAGAAGGACAAGATGGCGGAAGCCTACTCCGAGATAG
GGATGAAGGGCGAGCGGAGGCGGGGGAAGGGCCACGACGGGCTTTATCAAGGTCTGTCCACAGCCACCAAGGATAC
CTATGACGCTCTGCATATGCAGGCCCTGCCCCCACGCTAATAGAAAAACAAAAAACAAAAACCACACAAATGGTCG
CCGAAACTACTGAAAGCAT (SEQ ID NO: 180)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTTCCAGTGACAGCATTGTTGCTTCCTTTGGCACTGCTGTTACATGC
CGCCCGCCCCGACATTCAGATGACACAGACAACTTCCTCACTGAGTGCCAGTCTCGGAGACAGAGTGACCATCAGC
TGTCGGGCCTCCCAAGATATCAGTAAATACCTTAACTGGTACCAGCAGAAGCCCGATGGGACAGTTAAACTTCTCA
TCTATCATACCAGCAGGCTGCACAGCGGCGTTCCTAGCAGATTTAGTGGTAGCGGGAGCGGCACTGATTACAGCCT
GACAATTAGCAATCTCGAGCAGGAGGATATCGCTACCTACTTTTGCCAGCAGGGTAACACCCTGCCCTACACTTTC
GGGGGGGGAACTAAACTGGAGATAACAGGGGGTGGCGGGAGCGGGGGAGGTGGCAGTGGGGGGGGCGGGAGTGAAG
TGAAACTCCAGGAGAGCGGGCCAGGTCTCGTGGCCCCCTCTCAATCTTTGTCAGTGACTTGCACAGTCTCTGGCGT
TTCCCTGCCCGACTATGGCGTCTCATGGATTCGACAGCCCCCTCGAAAGGGGTTAGAGTGGCTCGGAGTGATATGG
GGCTCCGAAACCACCTATTACAATTCCGCTTTAAAGAGCAGATTGACAATCATCAAGGATAATTCCAAGTCTCAGG
TGTTTTTGAAAATGAACTCCCTTCAGACTGATGACACTGCAATCTACTACTGTGCCAAACATTACTATTACGGGGG
GAGTTACGCCATGGACTACTGGGGGCAGGGCACAAGTGTGACTGTCTCCTCCACCACCACTCCTGCTCCCCGACCT
CCTACACCTGCTCCAACTATCGCCTCTCAGCCACTGAGCCTGCGACCCGAGGCTTGCAGGCCTGCAGCTGGAGGCG
CTGTCCATACCAGAGGTCTGGACTTCGCCTGCGATATCTACATCTGGGCCCCACTAGCCGGTACCTGCGGCGTCCT
CTTATTGAGCCTCGTGATCACTCTTTACTGCAAGAGAGGCCGCAAGAAGCTGTTGTACATATTCAAACAGCCTTTT
ATGCGGCCTGTGCAGACTACACAGGAGGAAGACGGGTGCTCATGTAGATTCCCTGAGGAAGAGGAGGGTGGATGCG
AGCTTCGGGTGAAGTTCAGCCGAAGCGCTGACGCCCCCGCATACCAACAGGGCCAGAACCAACTCTACAATGAGTT
GAATTTGGGCCGCAGGGAAGAATACGACGTGCTGGATAAACGAAGGGGCCGAGACCCCGAAATGGGGGGCAAGCCT
CGGCGGAAGAATCCCCAGGAAGGCTTGTACAATGAGTTGCAGAAAGATAAAATGGCCGAGGCCTACTCCGAGATTG
GTATGAAGGGCGAAAGAAGGCGGGGAAAAGGGCACGACGGGTTGTATCAGGGGCTGAGTACCGCAACAAAGGACAC
CTATGATGCCCTACATATGCAGGCTCTACCTCCCAGATAATAGAAAAACAAAAAACAAAAACCACACAAATGGTCG
CCGAAACTACTGAAAGCAT (SEQ ID NO: 181)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTTCCCGTGACAGCCTTGCTGCTCCCATTGGCTCTCCTACTGCACGC
AGCACGACCCGATATTCAGATGACCCAGACTACCTCTTCCCTCAGCGCCTCCCTCGGGGACCGAGTGACTATCTCC
TGTCGGGCATCCCAGGATATTAGTAAGTACCTCAATTGGTACCAGCAAAAGCCCGACGGGACAGTCAAACTATTAA
TATACCACACCTCTAGACTACATAGTGGTGTTCCTTCTCGGTTCTCCGGGAGCGGCTCTGGGACTGACTACAGTCT
CACCATAAGCAACCTGGAGCAGGAGGACATAGCCACATACTTCTGTCAGCAGGGCAACACCCTGCCCTATACCTTC
GGAGGGGGCACCAAACTAGAGATTACTGGCGGAGGGGGTTCAGGAGGAGGCGGGAGCGGGGGCGGTGGAAGTGAGG
TGAAGTTACAGGAGTCCGGGCCTGGGCTCGTCGCCCCTTCCCAGAGCCTGTCAGTTACCTGTACTGTGAGCGGAGT
CTCATTACCCGACTACGGAGTCAGCTGGATCCGCCAGCCCCCCAGGAAGGGACTGGAGTGGCTGGGCGTGATCTGG
GGTTCTGAGACCACATATTATAATTCTGCTCTAAAATCTAGGCTGACAATCATCAAAGACAACAGCAAGAGCCAGG
TGTTTCTGAAGATGAACTCATTACAAACAGATGACACTGCTATCTACTACTGCGCAAAGCACTACTATTATGGTGG
GAGCTATGCCATGGATTATTGGGGGCAGGGGACCAGCGTCACTGTCTCCAGTACAACAACCCCCGCTCCACGACCT
CCAACCCCAGCACCCACTATTGCTTCACAGCCCCTGAGTTTACGGCCCGAGGCATGTCGCCCTGCCGCTGGAGGGG
CTGTTCATACCAGAGGACTGGACTTCGCCTGTGATATCTACATTTGGGCCCCACTGGCCGGAACATGTGGCGTTCT
CCTGCTCTCCCTCGTCATAACACTCTATTGCAAGAGAGGCAGGAAGAAACTTCTCTATATCTTTAAACAGCCTTTT
ATGAGGCCTGTGCAGACTACACAGGAAGAGGATGGATGTTCCTGCCGATTCCCAGAGGAGGAGGAGGGCGGGTGTG
AGCTACGAGTGAAGTTCTCACGCTCTGCAGATGCCCCCGCTTACCAGCAGGGGCAGAATCAGCTCTACAACGAGCT
GAATCTCGGACGCCGAGAAGAATACGACGTGCTGGACAAACGCAGGGGTAGAGACCCCGAGATGGGGGGAAAACCT
AGGCGCAAAAACCCCCAGGAGGGCCTGTATAACGAGCTGCAAAAGGATAAGATGGCCGAAGCTTACAGTGAGATCG
GCATGAAGGGGGAGCGCCGCCGCGGTAAAGGGCACGACGGTCTGTACCAGGGACTTAGCACAGCTACCAAGGACAC
CTACGATGCTCTCCACATGCAGGCATTACCACCCAGGTAATAGAAAAACAAAAAACAAAAACCACACAAATGGTCG
CCGAAACTACTGAAAGCAT (SEQ ID NO: 182)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCTTGCCCGTCACTGCCCTATTACTTCCTCTCGCCCTGTTGCTCCACGC
CGCTCGCCCCGACATCCAAATGACACAGACCACCTCTTCCCTATCCGCATCCTTGGGAGATAGGGTTACCATCTCC
TGTAGGGCATCCCAGGATATAAGCAAATATTTGAATTGGTATCAGCAAAAACCTGATGGTACAGTGAAGCTACTCA
TTTATCACACATCCCGGTTGCATTCCGGCGTGCCCTCCCGCTTCAGCGGAAGCGGTTCCGGGACTGACTATTCACT
TACTATAAGTAATCTAGAGCAAGAGGACATTGCAACTTATTTCTGCCAGCAAGGAAACACCCTACCCTACACATTT
GGAGGAGGAACAAAATTGGAAATCACCGGGGGCGGGGGTTCAGGTGGTGGTGGAAGCGGTGGTGGTGGATCAGAGG
TTAAGTTGCAAGAGAGCGGCCCCGGCCTTGTGGCTCCCAGTCAGAGTCTATCAGTGACCTGTACCGTGTCTGGCGT
TAGTCTCCCAGACTATGGAGTTAGCTGGATCAGACAACCACCACGCAAGGGACTGGAGTGGCTTGGAGTTATATGG
GGATCTGAGACCACATATTACAATTCTGCCCTTAAGTCCCGACTAACAATCATTAAAGACAACTCTAAGAGCCAGG
TTTTCCTCAAAATGAACTCTTTGCAGACCGATGACACCGCTATTTACTATTGTGCCAAGCACTACTATTACGGTGG
GTCATACGCTATGGATTACTGGGGGCAGGGCACATCAGTCACTGTGAGCTCTACAACCACACCCGCTCCCAGGCCT
CCAACCCCCGCCCCCACTATCGCCTCTCAACCTCTCAGCTTAAGGCCAGAGGCTTGTCGACCTGCTGCCGGCGGAG
CTGTTCATACCCGCGGTTTGGACTTCGCTTGCGACATATACATTTGGGCACCACTCGCCGGCACCTGCGGTGTGCT
CCTCCTTTCTCTCGTGATTACCCTGTATTGCAAGCGAGGCAGAAAAAAATTATTATATATTTTTAAACAGCCCTTC
ATGCGGCCTGTGCAGACTACACAAGAAGAAGATGGGTGCAGCTGCAGGTTCCCTGAGGAGGAGGAAGGCGGCTGTG
AACTTCGGGTCAAATTTTCCCGCAGTGCCGATGCTCCTGCATACCAACAAGGTCAAAACCAGCTGTACAATGAGTT
AAATCTTGGACGGAGAGAAGAGTATGATGTCTTGGACAAGCGGCGAGGTCGGGATCCTGAGATGGGCGGAAAGCCA
CGGCGCAAGAACCCACAAGAAGGTCTCTACAACGAGCTTCAGAAAGATAAAATGGCTGAAGCTTATAGCGAAATTG
GGATGAAAGGAGAAAGACGCAGGGGAAAGGGACACGACGGACTTTATCAAGGTCTATCTACCGCTACCAAAGATAC
TTATGACGCCCTCCACATGCAGGCACTTCCTCCACGCTAATAGAAAAACAAAAAACAAAAACCACACAAATGGTCG
CCGAAACTACTGAAAGCAT (SEQ ID NO: 183)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCTTGCCAGTAACCGCACTTCTGCTGCCACTAGCACTGCTGCTGCATGC
AGCCCGACCAGATATACAAATGACCCAGACAACCTCGAGTTTGAGTGCCTCCCTTGGAGATCGCGTAACTATCAGC
TGTCGGGCTAGTCAGGACATTTCAAAGTACCTCAACTGGTATCAGCAGAAACCTGATGGAACTGTGAAGCTGCTGA
TTTACCATACATCACGCTTACACTCCGGAGTGCCCTCACGTTTCTCCGGATCCGGTTCCGGAACTGATTATAGTCT
TACCATTAGCAACCTTGAGCAGGAAGACATAGCCACCTATTTCTGCCAACAAGGAAATACTTTGCCTTATACCTTT
GGGGGCGGCACTAAGCTGGAAATCACAGGTGGCGGGGGAAGCGGGGGCGGGGGAAGTGGCGGTGGGGGGTCTGAAG
TGAAGCTCCAAGAATCTGGACCAGGTTTAGTTGCACCGAGCCAAAGCCTATCCGTGACCTGCACTGTTTCCGGGGT
GTCTCTTCCAGATTACGGAGTGTCCTGGATACGCCAGCCGCCGCGAAAAGGGCTCGAGTGGTTGGGCGTCATATGG
GGCAGCGAAACGACCTATTATAACAGTGCCCTGAAAAGCCGGCTAACGATCATTAAAGATAACTCTAAGTCACAAG
TATTCCTCAAGATGAACTCCCTTCAGACGGATGATACGGCCATATACTACTGTGCTAAGCATTATTATTATGGCGG
GTCATACGCGATGGACTACTGGGGCCAGGGGACATCCGTCACCGTATCCTCTACGACGACACCGGCACCAAGACCT
CCTACTCCTGCACCCACCATAGCCAGCCAGCCTTTGTCACTGAGACCCGAGGCTTGTAGACCAGCTGCCGGTGGCG
CAGTGCACACCCGAGGCTTGGACTTTGCCTGTGACATTTACATCTGGGCACCACTTGCAGGGACTTGCGGGGTACT
CTTATTGAGCCTCGTAATCACACTGTACTGCAAGAGAGGACGCAAAAAATTGCTATATATTTTTAAACAGCCTTTT
ATGCGCCCTGTTCAGACTACGCAGGAGGAAGATGGATGTTCTTGTAGGTTTCCCGAAGAAGAGGAGGGCGGGTGTG
AGCTAAGAGTAAAATTCAGTAGATCTGCGGATGCACCGGCCTACCAGCAGGGTCAGAACCAGTTATACAACGAGCT
AAACTTAGGACGCAGAGAAGAGTACGATGTATTAGATAAACGCCGTGGTCGCGATCCTGAAATGGGTGGTAAGCCT
CGTCGGAAGAATCCTCAGGAGGGACTGTACAACGAACTACAGAAAGATAAGATGGCTGAGGCTTACTCCGAGATTG
GAATGAAGGGAGAGCGCAGAAGGGGAAAGGGACATGACGGCCTGTACCAAGGACTGAGCACTGCAACTAAGGATAC
CTACGACGCTCTGCACATGCAGGCCCTGCCCCCAAGATAATAGAAAAACAAAAAACAAAAACCACACAAATGGTCG
CCGAAACTACTGAAAGCAT (SEQ ID NO: 184)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTACCAGTCACAGCACTACTCCTCCCTCTTGCCCTCCTTCTTCACGC
CGCCAGGCCAGATATCCAGATGACACAGACCACCTCCTCCCTTTCCGCCTCTCTCGGGGACCGAGTCACTATTTCT
TGTCGGGCTTCTCAGGATATCAGTAAGTACCTTAATTGGTACCAGCAGAAGCCCGACGGGACAGTGAAGCTCCTGA
TCTACCACACCAGTAGGCTCCACTCTGGTGTCCCCTCCAGATTTAGTGGGTCAGGGAGTGGGACCGACTACTCCCT
GACCATCTCCAATCTGGAGCAGGAGGATATCGCCACCTACTTCTGTCAGCAGGGGAATACCCTGCCATATACATTT
GGAGGGGGCACCAAGCTGGAGATTACTGGTGGTGGTGGATCAGGAGGAGGTGGAAGTGGAGGAGGTGGTTCTGAAG
TGAAACTTCAGGAATCTGGACCTGGCCTGGTGGCTCCCAGCCAGTCCCTGAGTGTGACTTGCACCGTGTCTGGCGT
GTCTCTGCCAGACTACGGTGTGAGCTGGATCCGCCAGCCACCCAGGAAAGGCCTGGAGTGGCTGGGCGTGATCTGG
GGCAGTGAGACCACCTACTATAATAGTGCTTTGAAGTCCAGGCTGACCATCATCAAGGACAACTCCAAGAGTCAGG
TGTTCTTGAAGATGAACAGCCTGCAGACTGATGATACCGCAATCTACTATTGCGCAAAGCACTATTATTATGGGGG
GTCTTACGCCATGGATTACTGGGGGCAGGGGACTAGTGTTACTGTGAGTAGCACTACTACCCCTGCCCCTAGACCT
CCAACACCAGCTCCTACCATTGCAAGTCAGCCTCTGTCTCTGAGACCAGAGGCTTGTAGACCTGCAGCTGGTGGAG
CTGTGCATACTCGGGGACTGGATTTTGCATGTGACATTTACATCTGGGCTCCTCTGGCCGGCACCTGCGGCGTCCT
CCTCCTGAGTCTGGTGATCACTCTGTACTGCAAGCGCGGAAGGAAGAAGCTGCTGTACATCTTCAAGCAGCCCTTC
ATGCGCCCAGTGCAGACCACTCAGGAGGAGGACGGCTGCAGCTGCCGGTTTCCAGAGGAGGAAGAAGGAGGGTGTG
AGCTGAGGGTGAAGTTTTCACGCTCAGCTGACGCCCCTGCTTATCAGCAGGGCCAGAATCAGTTATATAATGAACT
GAATCTGGGCAGGAGGGAGGAGTACGATGTGCTGGATAAACGGAGAGGCAGGGATCCCGAAATGGGAGGTAAGCCT
CGGCGGAAGAATCCTCAGGAGGGTCTATATAACGAACTCCAGAAGGATAAGATGGCCGAGGCTTACTCCGAGATCG
GGATGAAAGGGGAGAGGCGCCGGGGTAAAGGCCACGATGGCCTTTACCAGGGCCTCTCCACCGCAACAAAGGATAC
CTATGATGCCCTGCATATGCAGGCCCTGCCTCCCCGGTAATAGAAAAACAAAAAACAAAAACCACACAAATGGTCG
CCGAAACTACTGAAAGCAT (SEQ ID NO: 185)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCACTACCCGTTACCGCTCTCTTGCTCCCTTTGGCCCTCCTCCTCCACGC
CGCCAGACCCGATATCCAGATGACTCAGACTACATCTAGTTTGAGTGCATCTCTCGGAGATCGGGTCACCATTTCT
TGTCGGGCTTCTCAGGATATCAGTAAGTACCTTAATTGGTACCAGCAGAAGCCCGACGGGACAGTGAAGCTCCTGA
TCTACCACACCAGTAGGCTCCACTCTGGTGTCCCCTCCAGATTTAGTGGGTCAGGGAGTGGGACCGACTACTCCCT
GACCATCTCCAATCTGGAGCAGGAGGATATCGCCACCTACTTCTGCCAGCAGGGGAATACCCTGCCATATACATTT
GGAGGGGGCACCAAGCTGGAGATTACTGGTGGTGGTGGATCAGGAGGTGGTGGTAGTGGAGGAGGAGGGTCAGAGG
TGAAACTGCAGGAGAGCGGTCCTGGTCTCGTTGCCCCAAGTCAGTCCCTGTCCGTGACATGTACCGTGTCTGGCGT
GTCTCTGCCAGACTACGGAGTGTCATGGATCAGACAGCCACCCAGGAAAGGCCTGGAGTGGCTGGGAGTGATTTGG
GGCAGCGAGACCACATATTATAACTCCGCCCTGAAGTCCAGGCTGACCATCATCAAGGACAACTCCAAGAGTCAGG
TGTTCTTGAAGATGAATAGCCTGCAGACAGATGACACGGCCATCTACTACTGTGCGAAGCACTACTACTATGGCGG
CAGCTACGCCATGGATTACTGGGGGCAGGGGACATCTGTGACTGTGAGTTCAACAACCACTCCAGCTCCACGGCCT
CCTACTCCAGCTCCAACCATTGCAAGTCAGCCTCTGAGTCTGAGACCAGAGGCTTGTAGACCTGCAGCTGGTGGAG
CTGTTCATACTCGCGGGCTGGATTTCGCCTGCGATATCTACATCTGGGCTCCTCTGGCCGGCACCTGCGGCGTCCT
CCTCCTGAGCCTGGTGATCACTCTGTACTGCAAGCGCGGAAGGAAGAAGCTGCTGTACATCTTCAAGCAGCCCTTC
ATGCGCCCAGTGCAGACCACTCAGGAGGAGGACGGCTGCAGCTGCCGGTTTCCAGAGGAGGAGGAAGGAGGCTGTG
AGCTGAGAGTGAAGTTTTCTCGCAGCGCAGATGCCCCTGCCTACCAGCAGGGCCAGAATCAGTTATATAATGAACT
GAATCTGGGCCGCAGGGAGGAGTATGATGTGCTGGATAAGCGGAGGGGCAGGGATCCCGAAATGGGGGGTAAGCCT
CGGCGGAAGAATCCTCAGGAGGGTCTATATAACGAACTCCAGAAGGATAAGATGGCCGAGGCTTACTCCGAGATCG
GGATGAAAGGGGAGAGGCGCCGGGGTAAAGGCCACGATGGCCTTTACCAGGGCCTCTCCACCGCAACAAAGGATAC
CTATGATGCCCTGCATATGCAGGCCCTGCCCCCCCGGTAATAGAAAAACAAAAAACAAAAACCACACAAATGGTCG
CCGAAACTACTGAAAGCAT (SEQ ID NO: 186)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTACCAGTGACAGCCCTCCTCCTCCCTTTGGCCTTGTTGCTCCACGC
AGCAAGGCCAGATATTCAGATGACACAGACAACCAGCAGCCTCTCCGCCTCTCTTGGAGACAGGGTGACCATTTCT
TGTCGGGCTTCTCAGGATATCAGTAAGTACCTTAATTGGTACCAGCAGAAGCCCGACGGGACAGTGAAGTTGCTGA
TCTACCACACTAGCAGGCTGCATAGCGGTGTCCCTTCCAGATTCTCGGGATCTGGAAGCGGCACCGACTACAGCCT
GACTATCAGCAACCTGGAGCAGGAGGACATCGCCACATATTTCTGCCAGCAGGGGAATACCCTGCCCTATACATTT
GGCGGGGGCACAAAGCTGGAGATTACAGGTGGCGGAGGCTCTGGAGGAGGAGGATCAGGCGGGGGCGGATCTGAAG
TGAAACTTCAGGAATCCGGCCCCGGCCTGGTCGCTCCTTCCCAGAGCCTGTCCGTCACCTGTACAGTCTCCGGCGT
GTCCCTGCCAGATTATGGGGTGAGTTGGATTCGCCAGCCCCCTAGAAAGGGGCTGGAATGGTTAGGCGTGATTTGG
GGGTCAGAGACCACCTACTACAACTCTGCCCTCAAGTCACGCCTGACCATTATCAAGGACAATAGTAAGAGCCAGG
TGTTTCTGAAGATGAATAGTCTTCAGACCGATGACACCGCTATTTACTATTGTGCCAAGCACTACTACTATGGCGG
CAGCTACGCCATGGATTACTGGGGGCAGGGGACTAGTGTTACTGTGAGTAGCACTACTACCCCTGCCCCTAGACCT
CCAACTCCAGCTCCAACCATTGCAAGTCAGCCTCTGAGTCTGAGACCAGAGGCTTGTAGACCTGCAGCTGGTGGAG
CTGTTCATACAAGGGGCCTGGATTTTGCCTGTGACATTTACATCTGGGCCCCTCTGGCAGGAACATGTGGCGTGCT
CCTCCTGTCTCTGGTGATCACCCTGTACTGTAAGAGAGGCAGGAAGAAGCTGCTGTACATCTTCAAGCAGCCATTC
ATGAGACCTGTGCAGACAACACAGGAAGAGGATGGCTGCTCCTGCAGGTTCCCTGAGGAGGAGGAGGGGGGCTGTG
AACTGCGCGTCAAATTCTCCCGGTCTGCCGATGCCCCTGCTTATCAGCAGGGGCAGAATCAGTTATATAATGAACT
GAATCTCGGCAGACGGGAGGAGTATGACGTGCTGGATAAGCGGAGGGGCAGGGATCCCGAAATGGGGGGTAAGCCT
CGGCGGAAGAATCCTCAGGAGGGTCTATATAACGAACTCCAGAAGGATAAGATGGCCGAGGCTTACTCCGAGATCG
GGATGAAAGGGGAGAGGCGCCGGGGTAAAGGCCACGATGGCCTTTACCAGGGCCTCTCCACCGCAACAAAGGATAC
CTATGATGCCCTGCATATGCAGGCCCTGCCCCCCCGGTAATAGAAAAACAAAAAACAAAAACCACACAAATGGTCG
CCGAAACTACTGAAAGCAT (SEQ ID NO: 187)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCAGTTACCGCCCTCCTCCTCCCCCTCGCCCTCCTCCTCCACGC
CGCCCGGCCCGACATCCAGATGACCCAGACCACCAGCAGCCTCAGCGCCAGCCTCGGCGACCGGGTGACCATCTCT
TGCCGGGCCAGCCAGGACATCAGCAAGTACCTCAATTGGTACCAGCAGAAGCCGGACGGCACCGTGAAGCTCCTGA
TCTACCACACCAGCCGGCTGCACTCCGGGGTGCCCAGCCGGTTCAGCGGCAGCGGCAGCGGCACCGACTACTCCCT
GACCATCAGCAACCTGGAGCAGGAGGACATCGCCACCTACTTCTGCCAGCAGGGGAACACCCTGCCCTACACCTTC
GGCGGCGGCACCAAGCTGGAGATCACCGGCGGCGGCGGCAGCGGCGGCGGCGGCAGCGGCGGGGGGGGCAGCGAGG
TGAAGCTGCAGGAGAGCGGCCCCGGCCTGGTGGCCCCCAGCCAGAGCCTGTCCGTGACCTGCACCGTGTCCGGCGT
GAGCCTGCCCGACTACGGCGTGTCCTGGATCCGCCAGCCCCCCAGGAAGGGCCTGGAGTGGCTGGGCGTGATCTGG
GGCAGCGAGACCACCTACTACAACAGCGCGCTGAAGTCCAGGCTGACCATCATCAAGGACAACAGCAAGAGCCAGG
TGTTCCTGAAGATGAACAGCCTGCAGACCGACGACACCGCCATCTACTACTGCGCCAAGCACTACTACTACGGCGG
CAGCTACGCCATGGACTACTGGGGCCAGGGCACCAGCGTGACCGTGTCCAGCACCACCACCCCCGCCCCCCGGCCC
CCCACCCCCGCCCCCACCATCGCCAGCCAGCCCCTGAGCCTGCGGCCGGAGGCCTGCAGGCCCGCCGCCGGCGGCG
CCGTGCACACCCGGGGCCTGGACTTCGCCTGCGACATCTACATCTGGGCCCCCCTGGCCGGCACCTGCGGCGTGCT
GCTGCTGAGCCTGGTGATCACCCTGTACTGCAAGCGGGGCCGGAAGAAGCTGCTGTACATCTTCAAGCAGCCCTTC
ATGCGCCCCGTGCAGACCACCCAGGAGGAGGACGGCTGCAGCTGCCGCTTCCCCGAGGAGGAGGAGGGCGGCTGCG
AGCTGCGGGTGAAGTTCTCCCGCAGCGCCGACGCCCCTGCGTACCAGCAGGGCCAGAACCAGCTGTACAACGAGCT
GAACCTGGGCCGGCGCGAGGAGTACGACGTGCTGGACAAGCGCCGGGGCCGGGACCCCGAGATGGGCGGCAAGCCC
CGGCGCAAGAACCCCCAGGAGGGCCTGTACAACGAGCTGCAGAAGGACAAGATGGCCGAGGCCTACAGCGAGATCG
GCATGAAGGGCGAGCGGCGGCGGGGCAAGGGCCACGACGGCCTGTACCAGGGCCTGTCCACCGCCACCAAGGACAC
CTACGACGCCCTGCACATGCAGGCCCTGCCCCCCCGGTAATAGAAAAACAAAAAACAAAAACCACACAAATGGTCG
CCGAAACTACTGAAAGCAT (SEQ ID NO: 188)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTGCCCGTGACCGCCCTGCTGCTCCCCCTGGCCCTGCTGCTGCACGC
AGCAAGGCCAGACATCCAGATGACCCAGACTACGAGCAGCCTGAGCGCCTCCCTGGGCGACAGGGTGACAATCAGC
TGCCGGGCCAGTCAGGACATCAGCAAGTACCTGAACTGGTACCAGCAGAAGCCTGACGGCACCGTGAAGCTGCTGA
TTTATCACACCTCCAGGCTGCACAGCGGCGTGCCCTCCAGATTCTCCGGCAGCGGCTCCGGCACCGACTACAGCCT
GACCATCTCCAACCTGGAGCAGGAGGATATCGCCACCTACTTCTGCCAGCAGGGGAACACCCTGCCCTATACCTTT
GGGGGCGGAACCAAGCTGGAGATCACCGGCGGTGGCGGATCCGGCGGCGGCGGGAGCGGCGGCGGAGGATCTGAGG
TCAAGCTGCAGGAGTCTGGCCCTGGGCTGGTGGCCCCAAGCCAGAGCCTGTCCGTGACATGTACCGTGTCCGGCGT
GTCTCTGCCTGACTACGGCGTGTCATGGATCAGGCAGCCTCCTCGCAAGGGGCTGGAGTGGCTGGGCGTGATCTGG
GGCAGCGAGACCACCTACTACAACAGTGCCCTGAAGTCCCGGCTGACCATCATCAAGGACAACAGCAAGAGCCAGG
TGTTCTTGAAGATGAACAGCCTCCAGACCGACGACACCGCCATTTACTACTGTGCCAAGCACTATTACTATGGCGG
GTCCTACGCCATGGATTACTGGGGCCAGGGCACCAGTGTGACCGTGTCCAGCACCACGACTCCTGCCCCACGCCCA
CCCACTCCAGCCCCTACCATCGCCAGCCAGCCACTGAGCCTGCGGCCTGAGGCCTGCAGGCCAGCGGCTGGCGGGG
CGGTGCACACCCGCGGGCTGGACTTCGCCTGCGACATCTACATCTGGGCCCCCCTCGCCGGCACCTGCGGCGTCCT
CCTCCTGAGCCTGGTGATCACTCTGTACTGCAAGAGGGGCCGCAAGAAGCTGCTGTATATCTTCAAGCAGCCTTTC
ATGCGGCCCGTGCAGACCACTCAGGAGGAGGACGGCTGCAGCTGCCGGTTTCCTGAGGAGGAGGAGGGGGGCTGTG
AGCTGCGGGTGAAGTTCAGCCGCTCCGCCGACGCCCCCGCCTACCAGCAGGGCCAGAATCAGCTGTACAACGAGCT
GAATCTGGGCCGCAGGGAGGAGTACGACGTGCTCGACAAGCGGCGGGGGCGGGACCCCGAGATGGGCGGCAAGCCC
AGGCGGAAAAACCCCCAGGAGGGGCTGTACAATGAGCTCCAGAAGGACAAAATGGCCGAGGCTTACAGCGAGATCG
GCATGAAGGGGGAGAGGCGGAGGGGCAAGGGGCATGACGGCCTGTACCAGGGGCTGTCCACAGCCACCAAGGACAC
CTACGACGCCCTGCACATGCAGGCCCTGCCTCCTAGGTAATAGAAAAACAAAAAACAAAAACCACACAAATGGTCG
CCGAAACTACTGAAAGCAT (SEQ ID NO: 189)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTGCCCGTGACCGCCCTGCTGCTTCCCCTGGCCCTGCTGCTGCACGC
CGCAAGGCCAGACATCCAGATGACCCAGACTACCAGTTCTCTGTCCGCCTCTCTGGGCGACAGAGTGACGATCAGT
TGCCGGGCCAGTCAGGACATCAGCAAGTACCTGAACTGGTACCAGCAGAAGCCTGACGGCACCGTGAAGCTGCTGA
TCTACCACACCAGCCGGCTGCACAGCGGAGTGCCCAGCCGGTTCAGCGGCTCCGGGTCCGGCACTGATTACAGCCT
GACCATCTCAAACCTGGAGCAGGAGGACATCGCCACTTACTTCTGCCAGCAGGGCAATACCCTGCCCTACACCTTC
GGAGGCGGCACCAAGCTGGAGATCACCGGCGGCGGCGGCTCCGGCGGCGGTGGCTCCGGCGGGGGCGGCTCCGAGG
TGAAGCTGCAGGAGAGTGGCCCAGGCCTGGTTGCCCCCAGCCAGAGCCTGAGCGTGACATGCACCGTGTCAGGCGT
GAGTCTTCCTGACTACGGGGTGAGCTGGATCAGGCAGCCCCCTAGGAAGGGCCTGGAGTGGCTGGGGGTGATCTGG
GGCTCAGAGACCACATACTACAACTCTGCCCTCAAGAGCAGGCTGACCATCATCAAGGACAACTCCAAGTCCCAGG
TGTTCTTGAAGATGAACAGCCTGCAGACCGACGATACCGCCATTTACTACTGTGCCAAGCACTACTACTATGGCGG
GTCCTACGCCATGGACTATTGGGGGCAGGGGACTAGTGTGACTGTGAGCTCCACCACCACCCCAGCCCCCAGGCCC
CCTACTCCTGCCCCTACCATCGCCAGCCAGCCCCTGAGCCTGCGGCCTGAGGCCTGCAGGCCAGCCGCCGGGGGCG
CCGTGCACACCAGGGGCCTGGACTTTGCCTGTGACATTTACATCTGGGCCCCTCTGGCCGGCACCTGCGGCGTGCT
GCTGCTGAGCCTGGTGATCACCCTGTACTGCAAGAGGGGCAGGAAGAAGCTGCTGTATATCTTCAAGCAGCCCTTC
ATGCGGCCAGTGCAGACCACTCAGGAGGAGGACGGCTGCAGCTGCCGCTTCCCTGAGGAGGAGGAGGGCGGCTGCG
AGCTGCGGGTGAAGTTCTCCCGCAGCGCCGATGCCCCTGCCTACCAGCAGGGCCAGAATCAGCTGTACAATGAGCT
GAATCTGGGCCGTCGGGAGGAGTACGACGTGCTGGATAAGCGCAGGGGCAGAGATCCTGAGATGGGCGGCAAGCCC
CGCAGGAAGAATCCTCAGGAGGGTCTGTACAATGAGCTGCAGAAGGACAAGATGGCTGAGGCTTATTCAGAGATCG
GCATGAAGGGCGAGAGGCGGCGGGGCAAGGGCCACGATGGCCTCTATCAGGGGCTGAGCACGGCCACCAAGGATAC
ATACGACGCCCTGCACATGCAGGCGCTGCCTCCCCGGTAATAGAAAAACAAAAAACAAAAACCACACAAATGGTCG
CCGAAACTACTGAAAGCAT (SEQ ID NO: 190)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTCCCCGTCACCGCCCTGCTCCTCCCCCTGGCACTCCTGCTGCACGC
CGCCAGGCCCGACATCCAGATGACCCAGACCACCAGCAGCCTCAGTGCCTCCCTGGGAGATAGGGTGACTATCTCC
TGCAGGGCCAGTCAGGACATCAGCAAGTACCTGAATTGGTACCAGCAGAAGCCTGACGGCACTGTGAAGCTGCTGA
TCTACCACACCTCCCGCCTGCACAGCGGTGTGCCCAGCCGGTTCTCCGGCTCTGGGTCCGGGACTGACTACAGCCT
GACCATCTCCAACCTGGAGCAGGAGGACATCGCCACCTACTTCTGCCAGCAGGGGAACACCCTGCCATACACCTTT
GGTGGCGGGACCAAGCTGGAGATCACCGGCGGAGGCGGGAGCGGCGGGGGCGGCTCCGGCGGAGGCGGGAGCGAGG
TCAAGCTGCAGGAGTCTGGCCCCGGCCTGGTGGCCCCCAGCCAGAGCCTGAGCGTGACCTGCACCGTCTCCGGCGT
GAGCCTGCCCGACTATGGCGTGAGCTGGATCAGGCAGCCACCCAGGAAGGGCCTGGAGTGGCTGGGCGTGATCTGG
GGGTCTGAGACCACCTACTATAACAGCGCCCTCAAGTCACGCCTGACCATCATCAAGGACAACAGCAAGTCCCAGG
TGTTCCTGAAGATGAACAGTCTGCAGACAGACGACACCGCCATCTACTACTGTGCCAAGCACTACTACTATGGCGG
GTCTTACGCCATGGACTACTGGGGGCAGGGGACGAGTGTGACTGTGTCCAGCACAACCACACCCGCCCCTAGGCCC
CCAACACCCGCCCCCACTATCGCAAGCCAGCCCCTGAGTCTGAGGCCCGAGGCCTGCCGGCCCGCCGCCGGCGGCG
CCGTGCACACCAGGGGCCTGGACTTCGCTTGCGATATCTACATCTGGGCTCCTCTGGCCGGAACCTGCGGCGTGCT
GCTGTTGTCCTTGGTGATCACTCTGTACTGCAAGAGGGGCCGCAAGAAGCTGCTGTACATCTTTAAGCAGCCTTTC
ATGCGGCCCGTGCAGACCACCCAGGAGGAGGACGGCTGCAGCTGCAGGTTCCCAGAGGAGGAGGAGGGCGGGTGTG
AGCTGAGGGTGAAGTTCTCCAGGTCTGCCGACGCCCCCGCCTACCAGCAGGGCCAGAATCAGCTCTACAATGAGCT
GAATCTGGGCCGCCGGGAGGAGTACGATGTGCTCGACAAGCGGCGGGGGCGCGACCCAGAGATGGGCGGCAAGCCT
AGGAGAAAGAACCCTCAGGAGGGGCTCTACAATGAGCTGCAGAAGGACAAGATGGCTGAGGCGTATTCAGAGATCG
GGATGAAAGGGGAGAGGAGGAGGGGCAAGGGGCACGACGGCCTGTATCAGGGGCTGAGCACCGCCACCAAGGACAC
CTATGACGCACTGCACATGCAGGCCCTGCCCCCGCGGTAATAGAAAAACAAAAAACAAAAACCACACAAATGGTCG
CCGAAACTACTGAAAGCAT (SEQ ID NO: 191)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTGCCCGTGACCGCCCTGCTTCTGCCCCTGGCCCTGCTGCTCCACGC
CGCCAGGCCAGACATCCAGATGACCCAGACAACCTCCAGCCTGAGCGCCAGCCTGGGCGACAGAGTGACCATCAGT
TGCCGGGCCAGCCAGGACATCAGCAAGTACCTGAATTGGTACCAGCAGAAGCCCGACGGCACCGTGAAGCTGCTGA
TCTACCACACCAGCCGCCTGCACAGCGGCGTGCCCAGCAGGTTCAGCGGCAGCGGCTCCGGCACCGACTACAGCCT
GACCATCAGCAACCTGGAGCAGGAGGACATCGCCACCTACTTCTGCCAGCAGGGCAATACCCTGCCCTACACCTTT
GGCGGCGGCACCAAGCTGGAGATCACCGGCGGCGGCGGAAGCGGCGGCGGCGGCTCCGGCGGGGGCGGCAGCGAGG
TGAAGCTGCAGGAGAGCGGCCCAGGCCTGGTGGCCCCCAGCCAGAGCCTGAGCGTGACCTGCACCGTGAGCGGCGT
GTCCCTGCCTGACTACGGGGTGAGCTGGATCAGGCAGCCCCCCAGAAAGGGCCTGGAGTGGCTGGGCGTGATCTGG
GGCAGCGAGACCACCTACTACAATTCCGCCCTGAAGTCCCGGCTGACCATCATCAAGGACAATTCCAAGAGCCAGG
TGTTCCTGAAGATGAACTCCCTGCAGACCGACGACACCGCCATCTACTACTGCGCCAAGCACTACTACTACGGCGG
CTCCTACGCCATGGACTACTGGGGCCAGGGCACCTCCGTGACCGTGAGCTCTACCACCACCCCCGCCCCTCGGCCC
CCAACACCTGCCCCCACCATCGCTAGCCAGCCCCTGTCTCTGAGACCCGAGGCTTGCAGACCAGCCGCCGGCGGCG
CCGTGCACACCAGAGGCCTGGACTTCGCCTGCGACATCTACATCTGGGCCCCCCTGGCCGGCACCTGCGGCGTGCT
GCTGCTGAGCCTGGTGATCACCCTGTACTGCAAGAGGGGAAGGAAGAAGCTGCTGTACATCTTTAAGCAGCCTTTC
ATGAGACCCGTCCAGACCACACAGGAGGAGGACGGCTGTAGCTGCAGGTTCCCAGAGGAGGAGGAGGGCGGCTGCG
AGCTGCGGGTGAAGTTCAGCAGGTCCGCTGACGCCCCCGCCTACCAGCAGGGCCAGAACCAGCTGTACAATGAGCT
GAACCTGGGCCGGAGAGAGGAGTACGACGTGCTGGACAAGAGGAGAGGCAGGGACCCCGAGATGGGCGGCAAGCCT
AGAAGGAAGAACCCCCAGGAGGGACTGTACAACGAGCTGCAGAAGGACAAGATGGCCGAGGCCTACTCTGAGATCG
GCATGAAGGGCGAGAGGAGAAGAGGCAAGGGCCACGATGGCCTGTACCAGGGCCTGAGCACCGCCACCAAGGACAC
CTACGACGCCCTGCACATGCAGGCCCTGCCCCCCCGGTAATAGAAAAACAAAAAACAAAAACCACACAAATGGTCG
CCGAAACTACTGAAAGCAT (SEQ ID NO: 192)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCAGTTACCGCCCTCCTCCTCCCCCTCGCCCTCCTCCTCCACGC
CGCCCGGCCAGATATCCAGATGACCCAGACCACCTCCTCCCTCAGCGCCTCCCTCGGCGACCGGGTGACCATCTCT
TGCCGGGCCAGCCAGGACATCAGCAAGTACCTCAATTGGTACCAGCAGAAGCCGGACGGCACCGTGAAGCTCCTGA
TCTACCACACCTCCCGCCTGCACAGCGGGGTGCCCAGCAGGTTCAGCGGCAGCGGCAGCGGCACCGACTACTCCCT
GACCATCAGCAACCTGGAGCAGGAGGACATCGCCACCTACTTCTGCCAGCAGGGGAACACCCTGCCCTACACCTTC
GGCGGCGGCACCAAGCTGGAGATCACCGGGGGCGGGGGCAGCGGGGGCGGGGGCAGCGGCGGGGGCGGCAGCGAGG
TGAAGCTGCAGGAGAGCGGCCCAGGCCTGGTGGCCCCCAGCCAGAGCCTGTCCGTGACCTGCACCGTGAGCGGCGT
GTCCCTGCCCGACTATGGCGTGAGCTGGATCCGCCAGCCACCCAGGAAGGGCCTGGAGTGGCTGGGCGTGATCTGG
GGCAGCGAGACCACCTACTACAACAGCGCCCTGAAGTCCAGGCTGACCATCATCAAGGACAACAGCAAGAGCCAGG
TGTTCCTGAAGATGAACAGCCTGCAGACCGACGACACCGCCATCTACTACTGCGCCAAGCACTACTACTACGGCGG
CAGCTACGCCATGGACTACTGGGGCCAGGGCACCTCCGTGACCGTGAGCAGCACCACCACCCCCGCCCCCCGCCCC
CCCACCCCCGCCCCCACCATCGCCAGCCAGCCGCTGAGCCTGCGGCCGGAGGCCTGCAGGCCCGCCGCCGGAGGCG
CCGTGCACACCCGGGGCCTGGACTTCGCCTGCGACATCTACATCTGGGCCCCCCTGGCCGGCACCTGCGGCGTGCT
GCTGCTGAGCCTGGTGATCACCCTGTACTGCAAGCGGGGCAGGAAGAAGCTGCTGTACATCTTCAAGCAGCCCTTC
ATGCGGCCCGTGCAGACCACCCAGGAGGAGGATGGCTGCAGCTGCAGGTTCCCTGAGGAGGAGGAGGGCGGCTGCG
AGCTGCGGGTGAAGTTCTCCCGCAGCGCCGACGCCCCCGCCTACCAGCAGGGCCAGAATCAGCTGTACAACGAGCT
GAATCTGGGCCGGCGGGAGGAGTACGACGTGCTGGACAAGCGCCGGGGCCGGGACCCCGAGATGGGCGGCAAGCCC
CGGCGCAAGAACCCCCAGGAGGGCCTGTACAACGAGCTGCAGAAGGATAAGATGGCCGAGGCCTACAGCGAGATCG
GCATGAAGGGCGAGCGGCGGAGGGGGAAGGGCCACGACGGCCTGTATCAGGGCCTGTCCACCGCCACCAAGGACAC
CTACGACGCCCTGCACATGCAGGCCCTGCCCCCCCGGTAATAGAAAAACAAAAAACAAAAACCACACAAATGGTCG
CCGAAACTACTGAAAGCAT (SEQ ID NO: 193)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCCGTGACGGCCCTCCTACTCCCCTTAGCTCTCTTGCTCCACGC
CGCCCGCCCGGACATCCAGATGACCCAGACGACCTCGAGCTTGAGTGCGTCACTCGGGGACCGAGTGACGATCTCT
TGCCGAGCGTCGCAGGACATCTCGAAGTACTTGAATTGGTACCAGCAGAAGCCCGACGGGACCGTTAAGCTATTAA
TCTACCACACGAGTAGATTGCATAGCGGCGTTCCCAGTCGGTTCTCTGGTAGCGGTTCAGGTACTGACTACTCGCT
GACCATCAGCAACCTGGAGCAGGAGGACATCGCCACCTACTTCTGCCAGCAGGGTAACACGCTTCCCTACACCTTC
GGTGGGGGGACCAAGCTGGAGATCACTGGTGGCGGAGGTAGCGGTGGCGGTGGTTCGGGCGGGGGCGGGAGCGAGG
TGAAGCTGCAGGAGAGTGGGCCTGGCCTCGTCGCGCCGTCACAGAGCCTCTCTGTGACGTGCACCGTGAGCGGGGT
CAGCCTGCCGGACTACGGAGTGTCCTGGATCCGGCAGCCGCCCCGCAAAGGCCTCGAGTGGCTCGGGGTCATTTGG
GGGAGCGAGACAACGTACTACAATAGTGCGTTGAAGTCGCGACTTACTATTATCAAGGATAATAGTAAGTCGCAGG
TCTTTCTGAAGATGAACTCACTTCAGACTGACGACACCGCAATATACTATTGCGCCAAGCACTACTACTATGGAGG
CAGTTACGCGATGGACTACTGGGGGCAGGGGACATCCGTGACTGTCTCCAGCACCACCACGCCGGCCCCGCGGCCC
CCCACCCCCGCGCCGACGATCGCGTCTCAGCCCCTCAGCCTGAGGCCTGAGGCGTGTCGTCCAGCGGCGGGTGGGG
CCGTGCACACACGGGGACTAGACTTTGCATGCGACATCTACATCTGGGCCCCGCTGGCGGGGACCTGTGGAGTGTT
GCTGCTGAGTCTAGTCATCACCTTGTACTGCAAGCGCGGCCGTAAGAAGCTCCTGTACATCTTCAAGCAGCCCTTC
ATGAGGCCTGTTCAAACGACGCAGGAGGAGGACGGGTGCAGCTGCCGCTTCCCTGAGGAGGAGGAGGGCGGCTGCG
AGCTGAGAGTGAAATTCTCACGCTCGGCGGACGCCCCAGCTTATCAGCAGGGCCAGAACCAGCTGTACAATGAGCT
GAACTTAGGCCGCAGGGAGGAGTACGACGTGCTCGACAAGCGCCGGGGTCGTGACCCGGAGATGGGTGGCAAGCCC
CGGCGCAAGAATCCGCAGGAGGGGCTCTACAACGAGCTCCAGAAGGACAAGATGGCGGAGGCCTACTCCGAGATTG
GCATGAAGGGGGAGCGCCGTCGTGGTAAGGGCCACGACGGCCTGTACCAGGGCCTGTCGACGGCCACTAAGGACAC
TTACGACGCCCTGCACATGCAGGCGTTGCCGCCCCGGTAATAGAAAAACAAAAAACAAAAACCACACAAATGGTCG
CCGAAACTACTGAAAGCAT (SEQ ID NO: 194)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCAGTTACAGCCCTGTTGCTGCCCCTGGCCCTGCTGCTGCACGC
CGCCCGGCCAGATATCCAGATGACGCAGACCACCAGCTCCCTGTCAGCCTCCCTGGGGGACAGGGTGACCATCAGC
TGCCGGGCCAGCCAGGACATCAGCAAGTACCTGAACTGGTACCAGCAGAAGCCTGATGGCACCGTGAAGCTGCTGA
TCTACCACACCTCCCGCCTGCACAGCGGGGTGCCCAGCAGGTTCAGCGGCAGCGGGTCAGGCACCGACTACAGCCT
GACCATCTCCAACCTGGAGCAGGAGGACATCGCCACCTACTTCTGCCAGCAGGGGAACACCCTGCCCTACACCTTC
GGCGGGGGCACCAAGCTGGAGATCACTGGCGGCGGGGGGAGCGGGGGGGGGGGGTCTGGGGGCGGGGGTAGTGAGG
TGAAGCTGCAGGAGAGCGGACCAGGCCTGGTGGCCCCCAGCCAGAGCCTGTCCGTGACCTGCACCGTGTCTGGCGT
GAGCCTGCCAGACTACGGCGTGTCCTGGATCCGCCAGCCACCCAGGAAGGGCCTGGAGTGGCTGGGCGTGATCTGG
GGCAGCGAGACCACCTACTACAACAGCGCGCTGAAGTCCAGGCTGACCATCATCAAGGACAACAGCAAGAGCCAGG
TGTTCCTGAAGATGAACAGCCTGCAGACCGACGACACCGCCATCTACTACTGTGCGAAGCACTACTACTATGGCGG
GTCCTACGCCATGGATTACTGGGGCCAGGGGACCTCAGTGACCGTGAGCAGCACCACTACCCCCGCCCCCAGACCC
CCCACCCCCGCTCCCACCATCGCCAGCCAGCCTCTGAGCCTGCGGCCAGAGGCCTGCAGGCCTGCCGCCGGGGGGG
CCGTGCACACCCGCGGGCTGGACTTCGCCTGCGACATCTACATCTGGGCCCCCCTGGCCGGCACCTGCGGGGTGCT
GCTGCTGAGCCTGGTGATCACCCTGTACTGCAAGCGGGGCCGGAAGAAGCTGCTGTACATCTTCAAGCAGCCCTTC
ATGCGCCCCGTGCAGACCACCCAGGAGGAGGACGGCTGCAGCTGCCGCTTCCCTGAGGAGGAGGAGGGCGGCTGCG
AGCTGCGGGTGAAGTTCTCCCGCAGCGCCGACGCCCCTGCGTACCAGCAGGGCCAGAATCAGCTGTACAACGAGCT
GAATCTGGGCAGGCGGGAGGAGTACGACGTGCTGGACAAGCGCCGGGGCCGGGACCCCGAGATGGGGGGCAAGCCC
CGGCGCAAGAACCCCCAGGAGGGGCTGTACAACGAGCTGCAGAAGGATAAGATGGCCGAGGCCTATTCCGAGATCG
GAATGAAGGGCGAGCGGCGGCGGGGCAAGGGGCACGACGGGCTGTACCAGGGCCTGAGCACCGCCACCAAGGACAC
CTACGATGCCCTGCACATGCAGGCCCTGCCCCCCCGGTAATAGAAAAACAAAAAACAAAAACCACACAAATGGTCG
CCGAAACTACTGAAAGCAT (SEQ ID NO: 195)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTACCAGTGACAGCCCTCCTCCTCCCACTCGCCCTCCTCTTGCACGC
CGCGCGCCCAGATATACAGATGACCCAGACGACGTCCAGCTTGAGTGCGTCACTCGGGGACCGAGTGACGATCTCT
TGCCGGGCGTCGCAGGACATCTCCAAGTACCTCAATTGGTACCAGCAGAAGCCAGACGGGACGGTGAAGCTCCTGA
TCTATCACACGTCCCGTCTGCATTCTGGTGTCCCGAGCCGGTTCTCCGGCTCGGGAAGTGGGACTGACTACAGTCT
CACGATATCCAACCTGGAGCAGGAGGATATCGCAACGTATTTCTGCCAGCAGGGAAATACGTTGCCGTACACCTTC
GGGGGAGGGACAAAGCTGGAGATCACTGGTGGGGGCGGGAGTGGGGGGGGGGGGAGCGGGGGAGGGGGCAGCGAGG
TCAAGCTCCAGGAGTCTGGGCCGGGCCTCGTTGCCCCCTCCCAGTCACTCTCAGTCACTTGCACCGTTTCCGGGGT
GTCGCTGCCGGACTACGGGGTGTCGTGGATCCGGCAGCCACCCCGGAAAGGGCTGGAGTGGCTGGGAGTGATCTGG
GGGTCCGAGACTACGTACTACAATAGTGCGTTGAAGTCTCGGCTCACCATAATAAAGGACAATAGTAAGTCGCAGG
TCTTTCTGAAGATGAACAGTCTTCAGACGGACGACACTGCGATTTACTATTGTGCCAAGCACTATTATTATGGTGG
GTCGTACGCCATGGATTACTGGGGTCAGGGGACCTCAGTGACCGTGAGCAGTACGACCACCCCCGCTCCCCGCCCC
CCCACTCCCGCCCCCACCATAGCATCCCAGCCTTTGTCCCTCCGCCCGGAGGCGTGCCGCCCCGCCGCGGGGGGGG
CGGTGCACACGCGAGGGCTGGACTTCGCGTGTGACATCTATATCTGGGCGCCGCTGGCGGGGACGTGCGGAGTTCT
TCTGCTCTCGCTTGTAATCACGCTGTATTGCAAGCGAGGGCGGAAGAAGCTCCTGTACATCTTCAAGCAGCCGTTC
ATGCGGCCGGTCCAGACGACCCAAGAGGAGGACGGGTGCTCCTGCCGGTTCCCGGAGGAGGAGGAGGGGGGCTGTG
AGCTGCGGGTCAAGTTCAGCCGTTCAGCTGATGCCCCTGCGTACCAGCAGGGGCAGAATCAGCTGTACAACGAGCT
GAACTTGGGCCGTCGAGAGGAGTACGACGTCCTCGACAAGCGTCGAGGACGTGATCCCGAGATGGGGGGTAAGCCC
CGCCGTAAGAATCCTCAGGAGGGGCTCTACAACGAGCTCCAGAAGGATAAGATGGCGGAGGCTTACTCCGAAATCG
GGATGAAGGGAGAGCGCCGTCGTGGCAAGGGCCACGACGGGCTCTACCAGGGCCTCTCGACGGCCACCAAGGACAC
CTACGACGCCCTGCACATGCAGGCGTTGCCGCCGCGGTAATAGAAAAACAAAAAACAAAAACCACACAAATGGTCG
CCGAAACTACTGAAAGCAT (SEQ ID NO: 196)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTACCCGTGACCGCTCTGCTGCTACCCCTGGCTCTGTTACTGCACGC
CGCCCGCCCTGACATTCAGATGACTCAGACTACCAGCAGCCTGAGCGCCTCGTTGGGAGACCGCGTTACCATCTCC
TGTCGCGCTAGCCAGGACATATCTAAATATCTCAATTGGTACCAACAGAAGCCTGACGGCACAGTTAAACTTCTCA
TTTATCATACCTCACGCCTCCATTCCGGAGTGCCCAGTAGGTTCAGTGGAAGCGGCAGTGGAACCGACTATAGCCT
CACTATTAGTAACTTGGAACAAGAGGACATAGCAACATATTTTTGCCAACAGGGGAATACCTTGCCATATACATTC
GGTGGGGGAACTAAACTCGAGATCACCGGCGGAGGTGGTAGCGGGGGAGGAGGATCAGGGGGAGGAGGTAGTGAGG
TGAAGTTGCAGGAGTCAGGTCCGGGACTGGTCGCGCCATCCCAGTCACTTTCAGTGACATGTACAGTGAGTGGTGT
TTCACTGCCCGACTACGGAGTCTCCTGGATCCGCCAGCCTCCCCGAAAGGGATTGGAGTGGCTTGGGGTAATATGG
GGGAGCGAAACGACCTACTATAACTCAGCCCTCAAAAGTAGGCTGACAATTATAAAGGATAACAGTAAAAGTCAAG
TTTTCCTTAAAATGAACAGCCTGCAGACAGATGATACGGCCATCTACTACTGTGCGAAACATTACTACTACGGGGG
CTCCTATGCCATGGATTATTGGGGACAGGGAACCTCAGTCACTGTGTCATCTACAACCACTCCGGCTCCACGGCCT
CCTACACCCGCACCCACTATTGCCTCACAGCCTCTCAGCCTGAGACCTGAAGCCTGCAGACCCGCTGCGGGCGGCG
CCGTACACACCCGAGGTCTGGATTTTGCCTGTGACATTTATATCTGGGCCCCGTTGGCTGGAACATGCGGTGTATT
ATTGCTGTCCCTGGTGATAACTTTGTACTGTAAGCGGGGAAGGAAGAAGCTACTGTACATCTTCAAGCAGCCCTTT
ATGCGCCCTGTGCAAACAACTCAAGAGGAGGATGGTTGTTCGTGCAGGTTCCCAGAGGAGGAGGAAGGCGGGTGTG
AGTTGCGTGTTAAGTTTTCGAGATCAGCTGACGCCCCTGCTTACCAGCAAGGGCAGAACCAGCTCTACAACGAGCT
GAATCTTGGACGTCGAGAAGAGTACGATGTTTTGGATAAAAGGCGTGGGCGCGATCCAGAGATGGGCGGTAAACCA
AGGCGTAAGAATCCTCAGGAGGGTCTCTATAACGAGTTGCAGAAGGATAAGATGGCTGAGGCTTACAGCGAAATCG
GAATGAAAGGAGAGAGACGGCGAGGTAAAGGTCACGATGGTCTCTATCAGGGATTGAGCACAGCGACCAAGGACAC
GTACGACGCCCTTCACATGCAAGCGCTGCCTCCGAGGTAATAGAAAAACAAAAAACAAAAACCACACAAATGGTCG
CCGAAACTACTGAAAGCAT (SEQ ID NO: 197)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCAGTAACCGCGCTTCTACTGCCATTAGCCTTGCTGCTCCACGC
AGCCCGACCCGACATTCAGATGACTCAGACTACTAGCTCTCTGAGTGCATCGCTAGGTGATCGTGTTACGATTTCC
TGCCGGGCCTCACAGGATATTTCTAAATACCTGAACTGGTACCAACAGAAGCCCGATGGGACGGTGAAGTTATTGA
TCTACCACACCAGTCGGTTGCACTCCGGCGTCCCATCCAGGTTTTCCGGGTCAGGCAGCGGTACTGACTATAGCCT
TACCATCAGCAACCTGGAACAGGAAGACATAGCCACCTACTTCTGCCAGCAAGGCAACACACTGCCCTACACCTTT
GGTGGCGGAACCAAGCTGGAGATTACTGGGGGCGGGGGTTCTGGTGGTGGTGGTTCGGGGGGGGGAGGGAGTGAGG
TCAAGCTTCAGGAATCGGGACCTGGTCTAGTGGCTCCATCTCAGTCCCTGTCCGTGACTTGCACTGTGTCCGGCGT
GTCGTTGCCGGACTACGGTGTCTCGTGGATCAGGCAACCCCCCCGCAAGGGCCTCGAGTGGCTAGGGGTGATTTGG
GGCTCCGAAACTACCTACTACAACTCCGCGCTCAAGTCTAGGCTGACGATCATCAAGGACAACTCTAAATCCCAAG
TGTTCTTAAAGATGAACAGTCTGCAAACAGACGATACCGCGATATATTATTGCGCTAAGCATTATTATTATGGTGG
CTCCTACGCCATGGATTACTGGGGTCAAGGCACTTCCGTTACAGTCAGTTCAACAACAACCCCAGCTCCACGTCCT
CCAACACCAGCCCCGACCATCGCCTCTCAGCCACTGTCCCTCAGGCCTGAGGCCTGCCGGCCTGCGGCCGGAGGGG
CAGTGCATACGCGGGGGTTGGACTTTGCTTGCGACATTTATATCTGGGCGCCCCTGGCCGGTACATGTGGAGTGCT
CCTTTTGTCCCTGGTGATCACCTTGTACTGCAAGAGAGGGAGAAAGAAGCTACTCTACATCTTTAAGCAGCCGTTC
ATGCGACCGGTCCAGACCACCCAGGAAGAGGACGGCTGCAGCTGCCGGTTTCCTGAAGAGGAAGAAGGCGGCTGCG
AATTACGCGTGAAGTTTTCGCGCAGCGCCGATGCTCCCGCTTATCAGCAAGGCCAAAACCAGCTCTACAATGAGTT
GAACCTGGGGCGACGTGAGGAGTATGATGTTCTTGATAAGCGGCGGGGCCGAGACCCTGAGATGGGCGGGAAACCG
CGACGTAAGAATCCTCAGGAGGGGCTGTACAACGAGCTCCAGAAAGATAAGATGGCGGAAGCGTATAGCGAGATCG
GTATGAAGGGGGAGCGCCGGCGCGGGAAGGGTCACGATGGACTGTACCAGGGCCTCAGTACCGCGACAAAGGATAC
CTATGACGCGTTGCATATGCAGGCCCTGCCCCCAAGGTAATAGAAAAACAAAAAACAAAAACCACACAAATGGTCG
CCGAAACTACTGAAAGCAT (SEQ ID NO: 198)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTGCCTGTGACCGCCCTGCTGCTGCCACTGGCCCTGCTGCTGCACGC
CGCCCGGCCAGATATCCAGATGACACAGACAACCTCCAGCCTGAGCGCCAGCCTGGGGGACAGAGTGACCATCAGC
TGCCGGGCCTCCCAAGACATCAGCAAATACCTGAACTGGTACCAGCAGAAACCCGATGGCACCGTGAAGCTGCTGA
TCTACCACACTTCCAGGCTGCACAGTGGGGTGCCCTCCCGGTTCTCCGGCAGCGGCAGCGGCACCGACTACAGCCT
GACCATCAGCAACCTGGAGCAGGAGGACATCGCCACCTACTTTTGCCAGCAGGGCAACACCCTGCCCTACACCTTC
GGCGGCGGCACCAAGCTGGAAATCACCGGGGGCGGGGGATCTGGGGGCGGCGGCTCCGGCGGCGGAGGATCCGAGG
TGAAGCTGCAGGAGTCCGGCCCAGGCCTGGTGGCCCCCAGCCAAAGTCTGTCCGTGACCTGTACCGTGTCCGGCGT
GTCCCTGCCCGACTACGGCGTGAGCTGGATTCGCCAGCCCCCCCGGAAGGGCCTGGAGTGGCTGGGCGTGATCTGG
GGCAGCGAGACCACCTATTACAACAGCGCCCTGAAGTCCAGGCTGACCATCATTAAAGATAATAGCAAGAGCCAAG
TGTTCCTCAAGATGAATAGCCTGCAGACCGACGATACCGCCATCTACTACTGTGCCAAACACTACTACTACGGCGG
GAGCTACGCCATGGACTACTGGGGCCAAGGCACCTCCGTGACCGTGAGCAGCACCACCACCCCAGCCCCCAGGCCC
CCTACCCCCGCTCCTACAATTGCCAGCCAGCCACTGTCCCTGCGCCCTGAGGCCTGCCGGCCCGCCGCCGGCGGCG
CCGTGCATACCCGGGGCCTGGACTTCGCCTGTGACATCTACATCTGGGCCCCCCTGGCTGGGACCTGCGGCGTGCT
GCTGCTGAGCCTGGTGATCACACTGTACTGCAAAAGGGGCCGGAAAAAACTGCTGTACATCTTCAAGCAGCCCTTC
ATGAGACCCGTGCAGACCACCCAGGAGGAGGACGGATGCTCCTGCCGCTTCCCAGAGGAGGAGGAGGGCGGGTGCG
AGCTGAGAGTGAAGTTTAGCCGCAGCGCCGATGCTCCCGCCTACCAGCAGGGCCAGAATCAGCTGTATAACGAACT
CAACCTGGGCAGAAGAGAAGAGTACGATGTGCTGGATAAAAGACGGGGCAGGGACCCCGAGATGGGCGGCAAGCCA
AGAAGAAAGAATCCCCAGGAGGGCCTGTACAACGAGCTGCAGAAGGATAAGATGGCCGAAGCCTACAGCGAGATTG
GCATGAAGGGAGAGCGGAGGCGCGGCAAGGGCCACGACGGCCTCTACCAGGGCCTGTCCACCGCCACCAAGGATAC
CTACGACGCTCTGCACATGCAAGCTCTGCCCCCCCGCTAATAGAAAAACAAAAAACAAAAACCACACAAATGGTCG
CCGAAACTACTGAAAGCAT (SEQ ID NO: 199)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTGCCCGTCACCGCCCTGCTGCTGCCCCTGGCCCTGCTGCTGCACGC
CGCCCGGCCCGATATCCAAATGACACAGACCACCAGCTCCCTGAGCGCCAGCCTGGGCGACAGAGTGACCATCTCC
TGCAGGGCTAGCCAGGACATCAGCAAGTATCTGAACTGGTACCAGCAGAAGCCCGACGGCACCGTGAAGCTCCTGA
TCTATCACACCTCCAGGCTGCACAGCGGCGTGCCCAGCAGATTCTCCGGCAGCGGCAGCGGCACCGACTACTCCCT
GACCATTAGCAACCTGGAGCAAGAGGACATCGCCACCTACTTCTGCCAGCAGGGCAACACACTGCCCTATACCTTT
GGCGGCGGCACCAAGCTGGAGATCACCGGCGGCGGCGGCAGCGGCGGCGGCGGCAGCGGCGGCGGCGGCTCCGAGG
TGAAACTGCAGGAAAGCGGCCCCGGCCTGGTGGCCCCCTCCCAGAGCCTGAGCGTGACCTGCACCGTGAGCGGCGT
GAGCCTGCCTGACTACGGCGTGTCCTGGATTAGGCAACCCCCCAGGAAGGGACTGGAGTGGCTCGGCGTGATTTGG
GGAAGCGAGACCACCTACTACAACTCCGCCCTGAAGTCCAGACTCACCATCATTAAAGATAACAGCAAATCCCAAG
TGTTCCTGAAGATGAACTCCCTGCAAACCGACGACACCGCCATCTACTACTGCGCCAAGCACTACTACTACGGAGG
CAGCTACGCCATGGACTATTGGGGCCAGGGCACCAGCGTGACCGTGAGCAGCACTACAACCCCCGCCCCCAGACCT
CCTACCCCTGCCCCCACCATCGCCTCACAGCCTCTGAGCCTGAGACCCGAGGCCTGCAGGCCCGCCGCCGGCGGCG
CCGTGCACACCCGGGGACTGGACTTCGCCTGCGACATTTACATCTGGGCCCCCCTGGCAGGCACCTGCGGCGTGCT
CCTGCTGAGCCTGGTGATTACCCTGTACTGCAAGAGAGGAAGAAAGAAACTGCTGTACATCTTCAAGCAGCCCTTC
ATGAGGCCCGTGCAGACCACCCAGGAAGAGGACGGCTGCTCCTGTAGGTTCCCCGAGGAGGAGGAGGGCGGCTGCG
AGCTCAGAGTGAAGTTTAGCAGGAGCGCCGACGCCCCCGCTTACCAGCAAGGCCAGAACCAGCTGTACAATGAACT
GAACCTGGGCAGACGGGAGGAATACGACGTGCTGGACAAAAGGAGAGGAAGAGACCCCGAGATGGGCGGAAAGCCC
CGGCGCAAGAACCCACAGGAGGGACTGTACAACGAGCTGCAAAAAGACAAGATGGCCGAAGCTTACAGCGAGATCG
GAATGAAGGGCGAGAGGAGACGCGGCAAGGGACACGATGGCCTCTACCAGGGCCTGAGCACCGCCACCAAAGACAC
CTATGATGCCCTCCACATGCAAGCCCTGCCCCCTAGGTAATAGAAAAACAAAAAACAAAAACCACACAAATGGTCG
CCGAAACTACTGAAAGCAT (SEQ ID NO: 200)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCTTACCAGTGACCGCCTTGCTCCTGCCGCTGGCCTTGCTGCTCCACGC
CGCCAGGCCGGACATCCAGATGACACAGACTACATCCTCCCTGTCTGCCTCTCTGGGAGACAGAGTCACCATCAGT
TGCAGGGCAAGTCAGGACATTAGTAAATATTTAAATTGGTATCAGCAGAAACCAGATGGAACTGTTAAACTCCTGA
TCTACCATACATCAAGATTACACTCAGGAGTCCCATCAAGGTTCAGTGGCAGTGGGTCTGGAACAGATTATTCTCT
CACCATTAGCAACCTGGAGCAAGAAGATATTGCCACTTACTTTTGCCAACAGGGTAATACGCTTCCGTACACGTTC
GGAGGGGGGACCAAGCTGGAGATCACAGGTGGCGGTGGCTCGGGCGGTGGTGGGTCGGGTGGCGGCGGATCTGAGG
TGAAACTGCAGGAGTCAGGACCTGGCCTGGTGGCGCCCTCACAGAGCCTGTCCGTCACATGCACTGTCTCAGGGGT
CTCATTACCCGACTATGGTGTAAGCTGGATTCGCCAGCCTCCACGAAAGGGTCTGGAGTGGCTGGGAGTAATATGG
GGTAGTGAAACCACATACTATAATTCAGCTCTCAAATCCAGACTGACCATCATCAAGGACAACTCCAAGAGCCAAG
TTTTCTTAAAAATGAACAGTCTGCAAACTGATGACACAGCCATTTACTACTGTGCCAAACATTATTACTACGGTGG
TAGCTATGCTATGGACTACTGGGGCCAAGGAACCTCAGTCACCGTCTCCTCAACCACGACGCCAGCGCCGCGACCA
CCAACACCGGCGCCCACCATCGCGTCGCAGCCCCTGTCCCTGCGCCCAGAGGCGTGCCGGCCAGCGGCGGGGGGCG
CAGTGCACACGAGGGGGCTGGACTTCGCCTGTGATATCTACATCTGGGCGCCCTTGGCCGGGACTTGTGGGGTCCT
TCTCCTGTCACTGGTTATCACCCTTTACTGCAAACGGGGCAGAAAGAAACTCCTGTATATATTCAAACAACCATTT
ATGAGACCAGTACAAACTACTCAAGAGGAAGATGGCTGTAGCTGCCGATTTCCAGAAGAAGAAGAAGGAGGATGTG
AACTGAGAGTGAAGTTCAGCAGGAGCGCAGACGCCCCCGCGTACCAGCAGGGCCAGAACCAGCTCTATAACGAGCT
CAATCTAGGACGAAGAGAGGAGTACGATGTTTTGGACAAGAGACGTGGCCGGGACCCTGAGATGGGGGGAAAGCCG
AGAAGGAAGAACCCTCAGGAAGGCCTGTACAATGAACTGCAGAAAGATAAGATGGCGGAGGCCTACAGTGAGATTG
GGATGAAAGGCGAGCGCCGGAGGGGCAAGGGGCACGATGGCCTTTACCAGGGTCTCAGTACAGCCACCAAGGACAC
CTACGACGCCCTTCACATGCAGGCCCTGCCCCCTCGCTAATAGAAAAACAAAAAACAAAAACCACACAAATGGTCG
CCGAAACTACTGAAAGCAT (SEQ ID NO: 201)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCACTTCCCGTTACCGCCCTCCTCCTTCCACTCGCCCTCCTCCTCCACGC
CGCCCGCCCAGATATACAGATGACACAGACCACCTCCAGCTTGAGTGCCTCCCTCGGGGACAGGGTGACCATCTCT
TGTCGGGCTTCTCAGGACATCAGCAAGTACCTCAATTGGTACCAGCAGAAGCCCGACGGGACCGTGAAGCTCCTGA
TCTACCACACCAGCAGGCTGCACAGCGGCGTGCCCTCCAGGTTTAGCGGGTCAGGGAGTGGGACCGACTACTCCCT
GACCATCTCAAACCTGGAGCAGGAGGACATCGCCACCTACTTTTGCCAGCAGGGCAACACCCTGCCCTACACCTTC
GGGGGAGGCACTAAGCTGGAGATCACTGGCGGTGGTGGGAGCGGGGGTGGGGGGTCTGGGGGCGGGGGTAGCGAGG
TGAAGCTGCAGGAGTCCGGACCCGGCCTGGTCGCCCCCAGCCAGTCCCTGTCCGTGACATGCACCGTGTCTGGCGT
GAGCCTGCCAGACTACGGCGTGTCATGGATCAGGCAGCCCCCCCGGAAGGGGCTGGAGTGGCTGGGGGTGATCTGG
GGGTCCGAGACTACCTACTACAACAGCGCGCTGAAGTCCAGGCTGACCATCATCAAGGACAACTCCAAGAGTCAGG
TGTTCTTGAAGATGAACAGCCTGCAGACCGATGACACCGCCATATACTACTGTGCGAAGCACTACTACTATGGCGG
GTCATACGCCATGGATTACTGGGGCCAGGGGACCTCAGTGACCGTGAGCAGCACCACCACCCCCGCCCCCAGACCC
CCCACCCCCGCTCCCACCATCGCCAGCCAGCCCCTGAGCCTGCGCCCCGAGGCCTGCCGGCCCGCGGCCGGCGGCG
CCGTGCACACTCGGGGGCTGGACTTCGCCTGTGACATCTATATCTGGGCCCCTCTGGCCGGCACCTGCGGCGTCCT
CCTCCTGAGTCTGGTGATCACCCTGTACTGCAAGCGCGGAAGGAAGAAGCTGCTGTACATCTTCAAGCAGCCCTTC
ATGCGCCCAGTGCAGACCACTCAGGAGGAGGACGGCTGCAGCTGCCGGTTCCCAGAGGAGGAGGAGGGCGGGTGCG
AGCTGCGGGTGAAGTTCTCCCGCAGCGCCGATGCCCCTGCGTACCAGCAGGGGCAGAATCAGCTCTACAACGAGCT
GAACCTGGGCCGCCGCGAGGAGTACGACGTGCTCGACAAGCGGCGGGGCCGGGATCCCGAGATGGGGGGTAAGCCT
CGGCGGAAGAATCCTCAGGAGGGGCTCTACAACGAGCTCCAGAAGGATAAGATGGCCGAGGCTTACTCCGAGATCG
GGATGAAGGGGGAGCGGCGGCGGGGCAAGGGGCACGACGGCCTGTATCAGGGCCTGAGTACCGCCACCAAGGATAC
TTACGACGCCCTGCACATGCAGGCCCTGCCCCCCCGGTAATAGAAAAACAAAAAACAAAAACCACACAAATGGTCG
CCGAAACTACTGAAAGCAT (SEQ ID NO: 202)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCCTACCAGTCACCGCCCTCCTCCTCCCCTTGGCCCTCCTCCTCCACGC
CGCCCGGCCAGATATTCAGATGACACAGACCACCTCCAGCCTTAGTGCCTCCCTCGGAGATAGGGTGACCATTTCT
TGTCGGGCTTCTCAGGACATCAGCAAGTACCTCAATTGGTACCAGCAGAAGCCCGACGGGACAGTGAAGTTGCTGA
TCTACCACACTAGCAGGCTGCATAGTGGCGTGCCCTCCCGCTTCTCTGGAAGCGGGAGCGGCACCGACTACAGCCT
GACTATCAGCAACCTGGAGCAGGAGGACATCGCCACCTACTTCTGCCAGCAGGGGAATACCCTGCCCTATACCTTC
GGGGGAGGCACTAAGCTGGAGATTACTGGCGGAGGCGGCAGTGGAGGAGGAGGGTCAGGGGGAGGAGGATCTGAAG
TGAAACTTCAGGAGAGTGGGCCCGGCCTTGTTGCTCCTTCTCAGAGCCTGTCCGTGACATGTACCGTGTCTGGCGT
GTCTCTGCCAGACTACGGCGTGTCATGGATCAGGCAGCCTCCTAGGAAGGGGCTGGAGTGGCTGGGCGTGATCTGG
GGTAGTGAGACTACTTACTACAATAGTGCCCTGAAGTCCCGGCTGACTATTATCAAGGATAATAGCAAGTCTCAGG
TGTTCCTGAAGATGAACAGCCTGCAGACAGATGACACGGCCATCTACTACTGTGCGAAGCACTACTACTATGGCGG
CAGCTACGCCATGGATTACTGGGGCCAGGGCACTAGTGTGACAGTGAGTAGTACCACTACCCCAGCTCCACGCCCA
CCTACTCCAGCCCCTACAATTGCCTCCCAGCCTCTGTCTCTGCGCCCTGAAGCTTGTCGCCCTGCTGCTGGTGGAG
CTGTGCACACCAGAGGGCTGGACTTCGCCTGCGACATCTATATCTGGGCTCCTCTGGCCGGCACCTGCGGCGTCCT
CCTCCTGAGTCTGGTGATCACCCTGTACTGCAAGCGCGGAAGGAAGAAGCTGCTGTACATCTTCAAGCAGCCCTTC
ATGCGCCCAGTGCAGACCACTCAGGAGGAGGACGGCTGCAGCTGCCGGTTTCCAGAGGAGGAAGAGGGTGGATGTG
AGCTGCGGGTGAAGTTCAGCCGCTCTGCTGATGCACCAGCCTACCAGCAGGGCCAGAATCAGCTCTACAACGAGCT
GAATCTGGGCCGCAGGGAGGAGTACGACGTGCTCGACAAGCGCAGGGGCAGAGATCCAGAGATGGGAGGCAAACCT
AGGAGGAAGAATCCCCAGGAGGGGCTCTACAACGAGCTGCAGAAGGACAAGATGGCCGAGGCCTACTCTGAGATCG
GAATGAAGGGCGAGAGGAGGCGGGGAAAAGGCCACGATGGCCTGTATCAGGGGCTGTCCACCGCCACCAAGGACAC
CTATGATGCCCTGCACATGCAGGCCCTGCCTCCTCGGTAATAGAAAAACAAAAAACAAAAACCACACAAATGGTCG
CCGAAACTACTGAAAGCAT (SEQ ID NO: 203)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTCCCAGTGACCGCCCTCCTCCTTCCCCTTGCCCTCCTCTTGCACGC
CGCTAGACCCGACATCCAGATGACTCAGACTACCTCTAGTTTGAGTGCATCCTTGGGTGATCGGGTCACCATCTCT
TGTCGGGCTTCTCAGGATATCAGTAAGTACCTTAATTGGTATCAGCAGAAGCCCGACGGGACCGTGAAGCTCTTGA
TCTACCATACATCAAGGCTTCACAGCGGCGTGCCCTCCAGGTTTAGCGGGTCAGGGAGTGGGACCGACTACTCCCT
GACCATCTCAAACCTGGAGCAGGAGGACATCGCTACTTACTTCTGCCAGCAGGGTAACACCCTGCCCTACACCTTT
GGGGGAGGGACAAAGCTGGAGATCACTGGTGGGGGCGGGAGTGGGGGGGGGGGGAGCGGGGGTGGGGGCAGCGAGG
TCAAGCTCCAGGAGTCTGGGCCTGGCCTCGTTGCCCCATCCCAGAGTCTGTCAGTGACATGCACTGTGAGTGGCGT
GTCACTGCCAGACTATGGGGTGTCCTGGATCCGCCAGCCACCCAGGAAGGGCCTGGAGTGGCTGGGCGTGATCTGG
GGGAGTGAGACCACCTACTATAATAGTGCTTTGAAGTCTAGACTTACTATTATCAAGGATAATAGTAAGTCTCAGG
TCTTTCTGAAGATGAACAGTCTTCAGACAGACGACACCGCAATATACTATTGCGCAAAGCACTATTATTATGGGGG
GTCTTACGCCATGGACTACTGGGGTCAGGGAACGTCCGTGACCGTCAGTAGTACCACCACCCCCGCTCCCCGCCCC
CCCACTCCCGCCCCCACCATTGCATCCCAGCCTTTGTCCCTCCGCCCAGAGGCCTGTAGGCCAGCCGCCGGGGGTG
CGGTGCACACCCGGGGGCTGGACTTCGCATGTGACATCTACATATGGGCTCCTCTGGCCGGCACCTGCGGCGTCCT
CCTCCTGAGTCTGGTGATCACCCTGTACTGCAAGCGCGGAAGGAAGAAGCTGCTGTACATCTTCAAGCAGCCCTTC
ATGCGCCCAGTGCAGACCACTCAGGAGGAGGACGGCTGCAGCTGCCGGTTCCCAGAGGAGGAGGAGGGGGGGTGTG
AGCTGAGGGTGAAGTTTTCACGCTCAGCTGACGCCCCCGCCTACCAGCAGGGCCAGAATCAGCTCTACAACGAGCT
GAATCTGGGCCGGCGGGAGGAGTACGATGTCCTCGACAAGAGGAGGGGGAGGGATCCCGAGATGGGGGGTAAGCCT
CGGCGGAAGAATCCTCAGGAGGGGCTCTACAACGAGCTCCAGAAGGATAAGATGGCCGAGGCTTACTCCGAGATCG
GGATGAAGGGGGAGCGGCGGAGGGGGAAGGGCCACGATGGACTGTATCAGGGGTTGTCCACTGCCACAAAGGACAC
CTATGATGCACTCCATATGCAGGCCCTGCCCCCTCGGTAATAGAAAAACAAAAAACAAAAACCACACAAATGGTCG
CCGAAACTACTGAAAGCAT (SEQ ID NO: 204)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTCCCAGTTACAGCCCTCTTGTTGCCTTTGGCCTTGCTCTTGCACGC
CGCAAGGCCCGATATCCAGATGACCCAGACCACCAGTTCCCTAAGCGCCAGCTTGGGGGACCGGGTGACCATCTCT
TGTCGGGCCAGTCAGGATATCAGCAAGTACCTCAATTGGTACCAGCAGAAGCCGGACGGCACCGTGAAGTTGCTGA
TCTATCACACCAGTAGGCTGCACTCCGGCGTGCCCTCCCGGTTCTCCGGGAGCGGCAGCGGGACAGACTACAGTCT
GACCATCAGCAACCTGGAGCAGGAGGACATTGCCACCTACTTCTGCCAGCAGGGGAACACCCTGCCCTATACATTT
GGCGGGGGCACCAAGCTGGAGATTACAGGCGGGGGAGGCTCTGGCGGCGGGGGCAGCGGGGGGGGCGGCTCTGAGG
TGAAGCTTCAGGAGTCCGGCCCCGGCCTCGTTGCCCCCAGCCAGAGCCTCTCCGTGACCTGTACCGTCTCCGGCGT
GTCCCTGCCAGATTATGGGGTGTCCTGGATCCGCCAGCCACCCCGGAAGGGGCTGGAGTGGCTGGGCGTGATCTGG
GGCTCAGAGACAACCTACTACAACTCTGCCCTGAAGAGTAGACTTACTATTATCAAGGATAATAGTAAGTCTCAGG
TGTTTCTGAAGATGAATAGCCTGCAGACAGATGACACGGCCATCTACTACTGCGCTAAGCACTACTACTATGGCGG
CAGCTATGCCATGGACTACTGGGGCCAGGGGACTAGCGTGACAGTGTCCTCCACCACCACCCCTGCTCCCAGGCCC
CCCACGCCTGCCCCCACCATCGCGAGCCAGCCACTGTCCCTCCGGCCAGAGGCCTGCCGGCCTGCTGCCGGAGGGG
CAGTGCACACAAGAGGGCTGGATTTCGCCTGTGACATTTACATCTGGGCTCCTCTGGCCGGCACCTGCGGCGTCCT
CCTCCTGAGCCTGGTGATCACCCTCTACTGCAAGAGGGGCAGGAAGAAGCTGCTGTACATCTTCAAGCAGCCTTTC
ATGAGGCCCGTGCAGACCACCCAGGAGGAGGACGGCTGCAGCTGCCGGTTCCCAGAGGAGGAGGAGGGCGGCTGCG
AGCTGCGGGTGAAGTTCTCCCGCAGCGCAGACGCCCCCGCCTACCAGCAGGGCCAGAATCAGCTGTACAACGAGCT
GAATCTGGGCCGGCGGGAGGAGTACGACGTGCTGGACAAGCGCCGGGGCCGGGACCCCGAGATGGGGGGCAAGCCC
CGGCGCAAGAATCCTCAGGAGGGGCTGTACAACGAGCTCCAGAAGGATAAGATGGCGGAGGCCTACAGCGAGATCG
GGATGAAGGGCGAGCGCAGGCGGGGCAAGGGGCATGACGGGCTGTACCAGGGCCTGTCAACTGCCACCAAGGACAC
CTACGACGCCCTGCACATGCAGGCCCTGCCTCCCCGGTAATAGAAAAACAAAAAACAAAAACCACACAAATGGTCG
CCGAAACTACTGAAAGCAT (SEQ ID NO: 205)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCTTACCAGTTACGGCGCTATTGCTCCCGTTGGCGCTATTGCTCCACGC
CGCGCGCCCGGACATACAGATGACGCAGACGACGTCTTCGTTATCGGCGAGTTTGGGCGACCGGGTTACGATATCG
TGCAGGGCTTCGCAGGACATAAGTAAGTACCTTAATTGGTACCAGCAGAAGCCCGACGGTACGGTAAAACTACTCA
TCTATCATACGTCGCGTTTGCATAGTGGAGTACCCTCCCGTTTTTCGGGGAGTGGGTCGGGTACCGACTATTCGCT
AACGATATCGAACTTGGAGCAAGAAGATATCGCGACGTACTTTTGCCAGCAAGGGAATACGTTACCGTACACGTTT
GGTGGAGGTACGAAACTAGAGATCACGGGTGGTGGTGGTTCGGGTGGTGGTGGTTCGGGTGGGGGCGGCTCGGAAG
TGAAACTTCAAGAGTCGGGTCCCGGTCTAGTGGCGCCGAGCCAATCGCTATCGGTAACGTGTACGGTTTCGGGTGT
ATCGCTACCGGATTACGGTGTATCGTGGATACGCCAACCGCCGCGTAAGGGCCTAGAATGGCTAGGGGTTATATGG
GGTAGTGAGACTACCTATTATAACTCGGCGTTGAAATCGCGTCTAACGATAATAAAAGATAATTCAAAATCGCAGG
TTTTTCTAAAAATGAATTCGCTACAAACGGATGATACGGCGATATATTATTGTGCGAAGCACTATTATTATGGTGG
TTCCTATGCGATGGATTATTGGGGTCAAGGTACGTCGGTAACCGTGTCGTCGACGACGACGCCGGCGCCGCGCCCG
CCGACGCCGGCGCCGACGATAGCGTCGCAACCGCTATCGTTGCGGCCGGAGGCGTGCCGCCCGGCGGCGGGGGGGG
CGGTGCATACGCGCGGCCTCGATTTTGCGTGTGACATTTACATATGGGCGCCGCTAGCGGGCACGTGTGGTGTCCT
ACTACTATCGTTGGTGATCACCCTATATTGTAAGCGTGGCAGGAAGAAGCTTCTTTATATATTTAAACAGCCGTTT
ATGCGGCCGGTACAAACGACGCAAGAAGAAGATGGTTGTTCGTGCCGCTTCCCGGAAGAAGAAGAAGGTGGCTGTG
AGCTACGCGTAAAGTTTTCGCGTTCGGCGGATGCTCCGGCGTATCAACAAGGTCAAAATCAGTTGTATAACGAACT
GAATCTAGGGCGTCGTGAAGAATACGACGTCCTAGATAAACGTCGTGGTCGGGACCCGGAAATGGGTGGTAAGCCG
CGTCGTAAAAACCCGCAAGAGGGGTTATATAATGAACTCCAAAAGGATAAAATGGCGGAGGCGTATTCGGAGATCG
GGATGAAAGGCGAGCGTCGCCGTGGTAAGGGCCACGACGGCCTTTATCAAGGTCTCTCGACGGCGACGAAGGATAC
GTATGATGCGTTGCATATGCAAGCGTTGCCGCCGCGTTAATAGAAAAACAAAAAACAAAAACCACACAAATGGTCG
CCGAAACTACTGAAAGCAT (SEQ ID NO: 206)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCACTACCGGTAACGGCGCTACTACTACCGCTAGCGTTACTATTGCATGC
AGCACGTCCGGATATACAAATGACGCAAACAACTTCGAGTTTATCGGCATCTTTAGGAGACCGTGTAACCATATCG
TGTCGAGCGTCTCAAGACATATCTAAGTATTTAAATTGGTATCAACAAAAACCTGACGGCACGGTAAAATTACTAA
TATACCATACGTCGCGACTCCATTCGGGTGTACCATCGCGTTTTTCGGGATCGGGTTCGGGTACTGATTACTCGCT
TACTATATCGAATTTAGAACAAGAAGATATCGCGACTTATTTTTGCCAACAAGGGAACACGCTACCTTATACCTTT
GGTGGAGGGACCAAATTGGAAATAACGGGGGGGGGGGGGAGTGGAGGAGGAGGTTCAGGGGGGGGGGGTTCAGAAG
TTAAATTACAAGAATCAGGACCAGGATTAGTAGCGCCGTCGCAATCGTTATCAGTTACGTGTACGGTCTCGGGTGT
AAGTCTACCGGATTACGGAGTATCGTGGATTAGGCAACCCCCGCGCAAGGGGCTTGAATGGCTTGGCGTTATCTGG
GGCTCGGAAACTACGTATTATAACTCGGCTCTAAAATCACGTTTGACGATAATAAAAGACAATTCGAAATCGCAAG
TATTCTTAAAAATGAACAGCCTACAAACGGACGACACGGCGATATACTATTGTGCGAAACATTATTATTATGGTGG
TAGTTATGCGATGGATTACTGGGGCCAAGGAACAAGCGTTACCGTATCGTCCACTACGACGCCTGCGCCGCGACCG
CCCACGCCGGCGCCTACCATAGCTTCACAACCATTAAGCCTCCGTCCAGAGGCGTGCCGTCCTGCGGCGGGTGGTG
CTGTACATACGAGGGGTCTAGACTTTGCGTGTGATATTTATATATGGGCTCCATTAGCGGGGACATGCGGGGTTCT
ACTACTATCGCTAGTAATAACTCTATACTGCAAACGTGGACGTAAAAAGTTACTATATATATTTAAACAGCCGTTC
ATGAGACCGGTACAAACTACGCAAGAGGAAGATGGGTGCTCCTGTAGATTTCCGGAGGAAGAAGAAGGTGGTTGCG
AATTAAGAGTTAAATTCAGTCGAAGTGCGGACGCGCCAGCGTATCAACAAGGGCAAAATCAACTATACAATGAACT
AAATCTTGGTCGAAGAGAAGAATATGACGTATTAGACAAACGGCGTGGACGAGACCCTGAGATGGGTGGAAAACCG
CGAAGAAAAAATCCACAAGAAGGTTTATACAATGAATTACAAAAAGATAAAATGGCGGAGGCATATAGTGAAATAG
GTATGAAAGGAGAACGAAGACGTGGTAAAGGGCATGACGGATTATATCAAGGATTGTCGACGGCAACGAAAGATAC
TTACGACGCGTTACATATGCAGGCCCTACCGCCGCGATAATAGAAAAACAAAAAACAAAAACCACACAAATGGTCG
CCGAAACTACTGAAAGCAT (SEQ ID NO: 207)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCCTTACCAGTAACAGCCTTATTACTTCCACTTGCCCTCCTTCTACATGC
CGCTAGACCAGATATCCAAATGACACAAACGACCTCAAGCCTTTCCGCGTCTCTCGGTGACAGGGTAACTATTAGT
TGTCGGGCCTCCCAAGATATTTCAAAATACTTAAATTGGTATCAACAGAAGCCAGATGGTACCGTGAAGCTCCTCA
TCTACCACACTAGCCGTCTTCACTCTGGAGTACCAAGCAGGTTTTCCGGCTCCGGAAGTGGTACTGACTACAGTTT
GACTATAAGCAACCTAGAGCAAGAGGATATTGCCACATACTTCTGTCAACAAGGAAACACTCTGCCATATACATTC
GGTGGTGGTACAAAACTGGAAATAACTGGAGGGGGAGGTTCAGGCGGCGGCGGATCTGGGGGGGGTGGCAGTGAGG
TCAAATTGCAGGAGTCTGGACCCGGTCTGGTTGCTCCATCCCAATCACTTTCAGTGACCTGCACTGTATCCGGAGT
TTCTCTGCCAGATTATGGAGTGAGTTGGATCCGCCAGCCTCCACGCAAGGGCCTTGAATGGCTCGGGGTTATATGG
GGTAGTGAAACTACATACTATAATAGCGCTCTGAAATCTCGACTCACAATCATTAAGGATAATAGCAAAAGCCAGG
TGTTTTTGAAGATGAATAGCCTGCAAACCGATGATACCGCCATATATTACTGTGCCAAACATTACTATTACGGAGG
CTCCTACGCTATGGATTATTGGGGACAGGGGACAAGCGTGACCGTTAGCTCTACTACTACCCCCGCCCCTCGCCCT
CCAACTCCAGCTCCCACCATTGCCAGTCAACCACTATCCTTAAGACCCGAGGCATGCAGACCCGCCGCAGGGGGCG
CCGTACATACCCGAGGTTTGGATTTTGCCTGCGACATCTATATCTGGGCCCCCCTTGCTGGGACATGTGGAGTGTT
GCTACTCTCGTTGGTTATCACTCTGTATTGCAAGAGAGGAAGAAAGAAACTATTATATATTTTTAAGCAGCCATTC
ATGAGACCGGTGCAGACTACCCAAGAAGAAGACGGATGTTCATGTAGGTTCCCTGAAGAAGAGGAAGGAGGCTGCG
AACTGCGAGTGAAATTTTCCAGATCTGCCGATGCACCAGCTTATCAACAAGGACAAAACCAGCTATATAATGAACT
TAATTTAGGGAGACGAGAGGAGTACGACGTCCTGGACAAACGCCGCGGAAGAGACCCTGAGATGGGAGGTAAGCCC
AGAAGAAAAAACCCACAAGAAGGACTGTACAATGAACTCCAAAAAGACAAAATGGCTGAAGCGTATTCCGAAATTG
GCATGAAAGGAGAACGGAGAAGAGGTAAGGGTCACGACGGCTTATATCAGGGACTCTCTACAGCAACCAAAGACAC
ATATGATGCACTACACATGCAGGCACTACCACCCAGGTAATAGAAAAACAAAAAACAAAAACCACACAAATGGTCG
CCGAAACTACTGAAAGCAT (SEQ ID NO: 208)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTACCAGTCACAGCACTACTATTGCCGTTGGCTCTATTGCTTCACGC
GGCGCGACCGGATATACAGATGACCCAAACGACGTCGAGTCTCTCGGCGTCGTTGGGTGATCGTGTAACGATATCG
TGTCGTGCGTCGCAAGATATCTCGAAATATCTAAATTGGTATCAACAAAAACCGGACGGGACGGTGAAATTATTAA
TATATCACACGTCCCGTCTACATTCTGGTGTTCCGAGTCGATTTAGTGGGAGTGGTTCGGGTACGGACTACTCCCT
AACTATATCGAACTTGGAACAAGAGGATATAGCGACGTACTTCTGCCAACAAGGTAATACGCTACCGTATACCTTT
GGCGGAGGTACGAAGCTAGAAATAACTGGTGGGGGTGGGAGTGGTGGTGGTGGTTCGGGTGGTGGTGGTTCTGAAG
TGAAACTTCAAGAATCGGGACCGGGGCTGGTAGCCCCGTCCCAATCGTTATCGGTAACGTGCACCGTATCGGGTGT
ATCGTTACCGGATTATGGTGTATCGTGGATACGCCAACCGCCCCGTAAAGGCCTAGAATGGCTAGGTGTTATATGG
GGTTCGGAAACGACGTACTACAATAGTGCGTTGAAATCGCGACTTACTATTATCAAGGATAATAGTAAGTCGCAAG
TATTTTTAAAGATGAATTCGTTACAGACGGACGATACCGCGATATACTATTGCGCGAAGCACTATTATTATGGTGG
TTCGTATGCGATGGATTATTGGGGTCAAGGTACGTCTGTAACGGTTTCATCTACTACTACTCCGGCACCACGACCA
CCAACTCCTGCTCCTACTATAGCGTCGCAACCGTTATCGTTGCGACCTGAAGCATGTAGACCAGCGGCAGGTGGTG
CTGTTCATACGCGAGGTCTAGACTTCGCGTGTGATATATATATATGGGCCCCGCTTGCGGGTACGTGTGGTGTACT
ATTACTATCGTTAGTAATAACGCTATACTGCAAGCGGGGCCGAAAAAAATTATTATATATATTTAAACAACCGTTT
ATGCGGCCGGTACAAACGACGCAAGAGGAAGATGGGTGTTCTTGTCGTTTTCCTGAAGAGGAAGAAGGTGGATGCG
AACTACGTGTAAAATTTTCGCGTAGTGCGGATGCGCCGGCCTATCAACAAGGCCAAAATCAACTCTATAACGAGTT
GAATCTAGGACGTCGTGAAGAGTACGACGTCCTAGATAAACGACGTGGTCGTGACCCGGAAATGGGTGGCAAACCG
CGTCGTAAAAACCCGCAAGAGGGTTTATATAATGAGCTACAAAAAGATAAAATGGCTGAAGCGTACTCGGAAATTG
GTATGAAAGGTGAACGTCGTCGTGGTAAAGGCCATGACGGTTTATATCAAGGTCTGAGTACGGCGACGAAGGACAC
CTATGATGCTCTACATATGCAAGCGTTACCGCCGCGTTAATAGAAAAACAAAAAACAAAAACCACACAAATGGTCG
CCGAAACTACTGAAAGCAT (SEQ ID NO: 209)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTACCCGTCACGGCGTTATTACTTCCGCTCGCGTTGCTATTACACGC
AGCGCGACCGGATATACAAATGACGCAAACGACGTCGTCGTTATCGGCGTCGCTAGGAGATCGTGTGACGATCTCT
TGCCGAGCGTCGCAAGATATCTCGAAATATCTAAATTGGTATCAACAAAAACCGGACGGTACGGTTAAATTGTTGA
TATATCATACGTCGAGGCTACATTCTGGTGTTCCGAGTCGATTTAGTGGGAGTGGTTCGGGTACGGACTACTCCCT
AACTATATCGAACTTGGAACAAGAGGATATAGCCACGTATTTTTGCCAACAGGGCAATACCCTCCCCTACACGTTT
GGGGGAGGGACGAAACTAGAAATTACAGGTGGTGGTGGTTCTGGGGGTGGTGGATCAGGTGGTGGTGGTTCTGAAG
TGAAACTTCAAGAATCAGGACCGGGTCTAGTCGCCCCCAGTCAATCGCTATCGGTAACCTGTACGGTTTCTGGTGT
CAGCTTACCGGATTATGGTGTAAGCTGGATACGTCAACCGCCCCGTAAAGGCCTAGAATGGCTAGGTGTTATATGG
GGTTCGGAGACGACGTACTACAATAGTGCGTTAAAATCGCGACTTACTATTATCAAAGATAATAGTAAGTCGCAGG
TTTTTTTAAAGATGAACTCTTTACAAACGGATGATACGGCGATATACTATTGCGCGAAGCACTATTATTATGGTGG
TTCGTATGCGATGGATTACTGGGGGCAGGGTACGTCGGTAACTGTGAGTTCGACTACGACTCCTGCTCCTAGACCG
CCGACTCCGGCGCCAACTATAGCATCGCAGCCTCTATCGCTTCGTCCGGAAGCGTGTAGACCAGCTGCAGGTGGTG
CTGTACATACGCGAGGTCTAGACTTCGCGTGTGATATATATATATGGGCGCCCCTAGCGGGTACGTGTGGGGTGCT
ATTACTATCGTTAGTAATAACGCTATATTGCAAACGTGGTAGGAAAAAGCTACTATATATATTTAAACAACCTTTT
ATGCGTCCGGTACAAACGACGCAAGAAGAGGATGGTTGTAGTTGTCGATTTCCGGAAGAAGAAGAGGGAGGTTGCG
AACTACGAGTAAAATTTAGTCGATCGGCGGATGCGCCGGCGTATCAACAAGGTCAAAATCAATTATATAATGAATT
AAATCTAGGACGTCGTGAAGAATATGACGTCCTAGATAAACGACGTGGTCGTGACCCAGAAATGGGTGGTAAACCG
CGGCGTAAAAACCCGCAAGAAGGTTTATATAATGAGCTTCAAAAAGATAAAATGGCGGAAGCTTATTCGGAAATAG
GTATGAAAGGTGAACGCCGTCGTGGTAAAGGGCATGACGGTCTTTACCAAGGTTTGTCGACGGCGACGAAAGATAC
GTATGATGCGTTACACATGCAAGCGTTACCGCCGCGTTAATAGAAAAACAAAAAACAAAAACCACACAAATGGTCG
CCGAAACTACTGAAAGCAT (SEQ ID NO: 210)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCCGTTACCGCCCTCCTCTTGCCCCTCGCCCTCTTGCTCCACGC
CGCCCGGCCAGATATCCAGATGACCCAGACCACCTCCTCCCTCAGCGCCTCCCTCGGCGACCGGGTGACCATCTCT
TGCCGGGCCTCACAGGACATCAGCAAGTACCTCAATTGGTACCAGCAGAAGCCCGACGGCACCGTGAAGCTTCTGA
TCTACCACACCTCCCGGCTGCACAGCGGGGTGCCCAGCAGGTTCAGCGGCAGCGGGTCAGGCACCGACTACAGCCT
GACCATCTCCAACCTGGAGCAGGAGGACATCGCCACCTACTTCTGCCAGCAGGGGAACACCCTGCCCTACACCTTC
GGCGGGGGCACCAAGCTGGAGATCACTGGCGGCGGGGGGAGCGGGGGGGGGGGGTCTGGGGGCGGGGGTAGTGAGG
TGAAGCTGCAGGAGAGCGGACCAGGCCTGGTGGCCCCCAGCCAGAGCCTGTCCGTGACCTGCACCGTGTCTGGCGT
GAGCCTGCCAGACTACGGCGTGTCCTGGATCCGCCAGCCACCCAGGAAGGGCCTGGAGTGGCTGGGCGTGATCTGG
GGCAGCGAGACCACCTACTACAACAGCGCGCTGAAGTCCAGGCTGACCATCATCAAGGACAACAGCAAGAGCCAGG
TGTTCCTGAAGATGAACAGCCTGCAGACCGACGACACCGCCATCTACTACTGTGCGAAGCACTACTACTATGGCGG
GTCCTACGCCATGGATTACTGGGGCCAGGGGACCTCAGTGACCGTGAGCAGCACCACTACCCCCGCCCCCAGACCC
CCCACCCCCGCTCCCACCATCGCCAGCCAGCCTCTGAGCCTGCGGCCAGAGGCCTGCAGGCCTGCCGCCGGGGGGG
CCGTGCACACCCGCGGGCTGGACTTCGCCTGCGACATCTACATCTGGGCCCCCCTGGCCGGCACCTGCGGGGTGCT
GCTGCTGAGCCTGGTGATCACCCTGTACTGCAAGCGCGGGAGGAAGAAGCTGCTGTACATCTTCAAGCAGCCCTTC
ATGAGGCCCGTGCAGACCACCCAGGAGGAGGATGGCTGCAGCTGCAGGTTCCCTGAGGAGGAGGAGGGCGGCTGCG
AGCTGCGGGTGAAGTTCTCCCGCAGCGCAGACGCCCCCGCCTACCAGCAGGGCCAGAATCAGCTGTACAACGAGCT
GAATCTGGGCCGGCGGGAGGAGTACGACGTGCTGGACAAGCGCCGGGGCCGGGACCCCGAGATGGGGGGCAAGCCC
CGGCGCAAGAATCCTCAGGAGGGCCTGTACAACGAGCTGCAGAAGGATAAGATGGCCGAGGCCTACAGCGAGATCG
GGATGAAGGGCGAGAGGAGGCGCGGCAAGGGGCACGACGGCCTGTATCAGGGCCTGTCCACCGCCACCAAGGACAC
CTACGACGCCCTGCACATGCAGGCCCTGCCCCCCCGGTAATAGAAAAACAAAAAACAAAAACCACACAAATGGTCG
CCGAAACTACTGAAAGCAT (SEQ ID NO: 211)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTACCAGTTACAGCCCTCTTGCTCCCTTTGGCCTTGTTGCTCCACGC
AGCAAGGCCAGACATCCAGATGACTCAGACTACCTCTAGTTTGAGTGCATCCTTGGGAGACCGGGTGACTATCAGT
TGCCGGGCCTCCCAGGACATCAGCAAGTACCTCAATTGGTACCAGCAGAAGCCGGACGGCACCGTGAAGTTGCTGA
TCTACCACACCAGCAGGCTGCACAGCGGCGTGCCCTCCCGGTTCTCCGGGAGCGGGTCAGGGACCGACTACTCCCT
GACCATCAGCAACCTGGAGCAGGAGGACATCGCTACTTACTTCTGCCAGCAGGGGAACACCCTGCCCTACACCTTT
GGCGGAGGGACAAAGCTGGAGATCACTGGTGGGGGCGGGAGTGGGGGGGGGGGGAGCGGGGGTGGGGGGTCTGAGG
TGAAGCTGCAGGAGTCCGGCCCCGGGCTGGTCGCCCCCTCACAGAGCCTGTCGGTGACATGCACTGTGAGCGGGGT
GAGCCTGCCCGACTACGGGGTGAGCTGGATCCGCCAGCCCCCCCGGAAGGGCCTGGAGTGGCTCGGAGTGATCTGG
GGCTCCGAGACCACTTACTACAACAGCGCCCTGAAGTCCAGGTTGACTATTATCAAGGATAATAGCAAGTCTCAGG
TGTTCCTGAAGATGAACAGCCTGCAGACCGATGACACCGCCATATACTACTGTGCGAAGCACTACTACTATGGCGG
GTCATACGCCATGGACTACTGGGGCCAGGGCACCTCAGTGACCGTCAGCTCCACCACCACCCCCGCTCCCCGCCCC
CCCACTCCCGCCCCCACCATCGCATCCCAGCCCCTGTCCCTCCGGCCAGAGGCCTGCCGGCCCGCCGCGGGCGGCG
CCGTGCACACCCGCGGGCTGGACTTCGCCTGCGACATCTACATCTGGGCTCCTCTGGCCGGCACCTGCGGCGTCCT
CCTCCTGAGCCTGGTGATCACTCTGTACTGCAAGCGCGGAAGGAAGAAGCTGCTGTACATCTTCAAGCAGCCCTTC
ATGCGCCCAGTGCAGACCACCCAGGAGGAGGACGGCTGCAGCTGCCGGTTCCCAGAGGAGGAGGAGGGCGGGTGCG
AGCTGCGGGTGAAGTTCTCCCGCAGCGCCGACGCACCCGCCTACCAGCAGGGCCAGAATCAGCTCTACAACGAGCT
GAATCTGGGCCGGCGGGAGGAGTACGATGTGCTGGACAAGCGGAGGGGCAGGGATCCCGAGATGGGGGGTAAGCCT
CGGCGGAAGAATCCTCAGGAGGGGCTCTACAACGAGCTCCAGAAGGATAAGATGGCCGAGGCTTACTCCGAGATCG
GGATGAAGGGGGAGAGGCGCCGGGGTAAGGGCCACGATGGCCTTTACCAGGGCCTCTCCACCGCAACAAAGGACAC
CTATGATGCCCTGCACATGCAGGCCCTGCCCCCCCGGTAATAGAAAAACAAAAAACAAAAACCACACAAATGGTCG
CCGAAACTACTGAAAGCAT (SEQ ID NO: 212)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTTTACCCGTTACAGCCCTGTTGCTGCCCCTTGCCCTGCTGCTGCACGC
CGCCCGGCCAGATATCCAGATGACGCAGACCACCAGCTCATTGTCAGCCTCCCTGGGGGATCGGGTGACCATCAGC
TGCCGGGCCAGTCAGGACATCAGCAAGTACCTGAACTGGTACCAGCAGAAGCCTGATGGCACCGTGAAGCTGCTGA
TCTACCACACCAGCAGGCTTCACTCCGGCGTGCCTTCCCGCTTCTCTGGGTCCGGCTCCGGCACAGACTACAGTCT
GACCATCTCCAACCTGGAGCAGGAGGACATCGCTACTTACTTCTGCCAGCAGGGGAACACCCTGCCCTACACCTTC
GGCGGGGGCACCAAGCTGGAGATCACTGGCGGTGGTGGGAGCGGGGGTGGGGGGTCTGGGGGCGGGGGTAGCGAGG
TGAAGCTGCAGGAGTCCGGACCCGGCCTGGTCGCCCCCAGCCAGTCCCTGTCCGTGACATGCACCGTGTCTGGCGT
GAGCCTGCCAGACTACGGCGTGTCATGGATCAGGCAGCCCCCCCGGAAGGGGCTGGAGTGGCTGGGGGTGATCTGG
GGGTCCGAGACTACCTACTACAACAGCGCGCTGAAGTCCAGGCTGACCATCATCAAGGACAACTCCAAGAGTCAGG
TGTTCTTGAAGATGAACAGCCTGCAGACCGATGACACCGCCATATACTACTGTGCGAAGCACTACTACTATGGCGG
GTCATACGCCATGGATTACTGGGGCCAGGGGACCTCAGTGACCGTGAGCAGCACCACCACCCCCGCCCCCAGACCC
CCCACCCCCGCTCCCACCATCGCCAGCCAGCCCCTGAGCCTGCGCCCCGAGGCCTGCCGGCCCGCGGCCGGCGGCG
CCGTGCACACTCGGGGGCTGGACTTCGCCTGCGATATCTATATCTGGGCCCCTCTGGCCGGCACCTGCGGCGTCCT
CCTCCTGAGTCTGGTGATCACCCTGTACTGCAAGCGCGGAAGGAAGAAGCTGCTGTACATCTTCAAGCAGCCCTTC
ATGCGCCCAGTGCAGACCACTCAGGAGGAGGACGGCTGCAGCTGCCGGTTCCCAGAGGAGGAGGAGGGGGGCTGCG
AGCTGCGGGTGAAGTTCTCCCGCAGCGCAGACGCCCCCGCCTACCAGCAGGGCCAGAATCAGCTCTACAACGAGCT
GAATCTGGGCCGGCGGGAGGAGTACGATGTCCTGGACAAGCGCCGGGGCCGGGACCCAGAGATGGGCGGGAAGCCA
CGCCGGAAGAACCCCCAGGAGGGGCTGTACAATGAGCTGCAGAAGGATAAGATGGCCGAGGCCTATTCCGAGATCG
GAATGAAGGGCGAGCGGCGGCGGGGCAAGGGGCACGACGGGCTGTACCAGGGGCTGAGCACCGCAACAAAGGACAC
CTATGATGCCCTGCACATGCAGGCTCTGCCCCCCCGGTAATAGAAAAACAAAAAACAAAAACCACACAAATGGTCG
CCGAAACTACTGAAAGCAT (SEQ ID NO: 213)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTTGCTCAGCGTAACTTCTCCGGGTTACGTGGAGACCAAAAGGC
TACGGAGACTCGGGCTACGGCCCTGGAGCACCTAGGTGCTCCTAAAGACGTTAGAAGTTGTACAAACTCGCCCAAT
AGGGCCCCCCAACCAGGGGGGTAGCGGGCAAGCACTTCTGTTTCCCCGGTATGATCTCATAGGCTGTACCCACGGC
TGAAAGAGAGATTATCGTTACCCGCCTCACTACTTCGAGAAGCCCAGTAATGGTTCATGAAGTTGATCTCGTTGAC
CCGGTGTTTCCCCCACACCAGAAACCTGTGATGGGGGTGGTCATCCCGGTCATGGCGACATGACGGACCTCCCCGC
GCCGGCACAGGGCCTCTTCGGAGGACGAGTGACATGGATTCAACCGTGAAGAGCCTATTGAGCTAGTGTTGATTCC
TCCGCCCCCGTGAATGCGGCTAATCCCAACTCCGGAGCAGGCGGGCCCAAACCAGGGTCTGGCCTGTCGTAACGCG
AAAGTCTGGAGCGGAACCGACTACTTTCGGGAAGGCGTGTTTCCTTTTGTTCCTTTTATCAAGTTTTATGGTGACA
ACTCCTGGTAGACGTTTTATTGCGTTTATTGAGAGATTTCCAACAATTGAACAGACTAGAACCACTTGTTTTATCA
AACCCTCACAGAATAAGATAACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCTAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAAGAGAGGCCGGAAGAAGCTGCTCTACATCTTCAAGCAGCCCTTC
ATGAGGCCCGTGCAGACCACACAAGAAGAGGACGGCTGCTCCTGCAGATTCCCCGAGGAAGAGGAAGGCGGCTGCG
AGCTGAGAGTGAAGTTCAGCAGAAGCGCTGACGCCCCTGCTTACCAGCAGGGACAGAACCAGCTGTACAACGAGCT
GAACCTGGGGAGAAGAGAAGAGTACGACGTGCTGGACAAGCGGAGAGGCAGAGATCCTGAGATGGGCGGCAAGCCC
AGAAGAAAGAACCCTCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTACCAGGGCCTGAGCACAGCCACCAAGGACAC
ATATGACGCCCTGCACATGCAGGCCCTGCCTCCAAGATAATAGAAAAACAAAAAACAAAAACCACACAAATGGTCG
CCGAAACTACTGAAAGCAT (SEQ ID NO: 214)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTGAACCGTTACGCACCACTCAGTTGGTGTTTGGTGGCACCAATG
ATGGAACAAAAGGCTACACCACTTGGGCTACGGCCCGCGCCACCTTGTGGCGCAAAGACATTAGAAGAATAGCATA
CCGCCCACTAGGGCCCTGCAGCCAGCAGGGTAACGGGCAAGCACTTCTGTCTCCCCGGTAGAACGGTATAGGCTGT
ACCCACGGCCGAAAACTGAACTATCGTTACCCGACTCCGTACTTCGCAAAGCTTAGTAGGAAACTGGAAAGTTCGA
GTTATTGACCCGGAGTGTTCCCCCCACTCCAGAAACGCGTGATGAGGGTTGCCACCCCGACCATGGCGACATGGTG
GGCATCCCTGCGCTGGCACGCGGCCTCTAAGAGGATAACTCGCTCCTACTGGTAACCGAAGAGCCCCGTGAGCTAC
GGTTTATTCCTCCGCCTCCCTGAATGCGGCTAATCCTAACCCATGAGCAGTTGCCATAGATCCATATGGTGGACTG
TCGTAACGCGTAAGTTGTGGGCGGAACCGACTACTTTGGGATGGCGTGTTTCCTTGTTTTCTCCATTTGTTGTTGT
ATGGTGACAAGTTATAGATCTCGATCTATAGCGTTTCTTGAGAGATTTCCAAACATTTATTCAAGTCGTACAATTC
TTGTGTTTAAGCAGTACAGTGTAAGGATGGCTCTGCCTGTGACAGCTCTGCTGCTGCCTCTGGCTCTGCTTCTGCA
TGCCGCCAGACCTGACATCCAGATGACCCAGACAACCAGCAGCCTGTCTGCCAGCCTGGGCGACAGAGTGACCATC
AGCTGTAGAGCCAGCCAGGACATCAGCAAGTACCTGAACTGGTATCAGCAGAAACCCGACGGCACCGTGAAGCTGC
TGATCTACCACACCAGCAGACTGCACAGCGGCGTGCCATCTAGATTTTCTGGCAGCGGCTCTGGCACCGACTACAG
CCTGACAATCAGCAACCTGGAACAAGAGGATATCGCTACCTACTTCTGCCAGCAAGGCAACACCCTGCCTTACACA
TTCGGCGGAGGCACCAAGCTGGAAATCACAGGTGGCGGAGGATCTGGCGGAGGTGGAAGCGGAGGCGGTGGATCTG
AAGTGAAACTGCAAGAGTCTGGCCCTGGCCTGGTGGCTCCATCTCAGTCTCTGAGCGTGACCTGTACCGTCAGCGG
AGTGTCTCTGCCTGATTACGGCGTGTCCTGGATCAGACAGCCTCCTAGAAAAGGCCTGGAATGGCTGGGCGTGATC
TGGGGCAGCGAGACAACCTACTACAACAGCGCCCTGAAGTCCAGGCTGACCATCATCAAGGACAACTCCAAGAGCC
AGGTGTTCCTGAAGATGAACAGCCTGCAGACCGACGACACCGCCATCTACTACTGCGCCAAGCACTACTACTACGG
CGGCAGCTACGCCATGGATTACTGGGGCCAGGGAACCAGCGTGACCGTGTCTAGCACAACAACCCCTGCTCCTAGA
CCTCCTACACCAGCTCCTACAATCGCCAGCCAGCCTCTGTCTCTGAGGCCAGAGGCTTGTAGACCTGCTGCTGGCG
GAGCTGTGCACACAAGAGGCCTGGATTTCGCCTGCGACATCTACATCTGGGCCCCTCTGGCTGGAACATGTGGCGT
GCTGCTGCTGAGCCTGGTCATCACCCTGTATTGCAAGAGAGGCCGGAAGAAGCTGCTCTACATCTTCAAGCAGCCC
TTCATGAGGCCCGTGCAGACCACACAAGAAGAGGACGGCTGCTCCTGCAGATTCCCCGAGGAAGAGGAAGGCGGCT
GCGAGCTGAGAGTGAAGTTCAGCAGAAGCGCTGACGCCCCTGCTTACCAGCAGGGACAGAACCAGCTGTACAACGA
GCTGAACCTGGGGAGAAGAGAAGAGTACGACGTGCTGGACAAGCGGAGAGGCAGAGATCCTGAGATGGGCGGCAAG
CCCAGAAGAAAGAACCCTCAAGAGGGCCTGTATAATGAGCTGCAGAAAGACAAGATGGCCGAGGCCTACAGCGAGA
TCGGAATGAAGGGCGAGCGCAGAAGAGGCAAGGGACACGATGGACTGTACCAGGGCCTGAGCACAGCCACCAAGGA
CACATATGACGCCCTGCACATGCAGGCCCTGCCTCCAAGATAATAGAAAAACAAAAAACAAAAACCACACAAATGG
TCGCCGAAACTACTGAAAGCAT (SEQ ID NO: 215)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCTAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAGAGTGAAGTTCAGCAGAAGCGCTGACGCCCCTGCTTACCAGCAG
GGACAGAACCAGCTGTTTAACGAGCTGAACCTGGGGAGAAGAGAAGAGTTCGACGTGCTGGACAAGCGGAGAGGCA
GAGATCCTGAGATGGGCGGCAAGCCCAGAAGAAAGAACCCTCAAGAGGGCCTGTTTAATGAGCTGCAGAAAGACAA
GATGGCCGAGGCCTTCAGCGAGATCGGAATGAAGGGCGAGCGCAGAAGAGGCAAGGGACACGATGGACTGTTCCAG
GGCCTGAGCACAGCCACCAAGGACACATTCGACGCCCTGCACATGCAGGCCCTGCCTCCAAGATAATAGAAAAACA
AAAAACAAAAACCACACAAATGGTCGCCGAAACTACTGAAAGCAT (SEQ ID NO: 216)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTAAAACAGCCTGTGGGTTGATCCCACCCACAGGCCCATTGGGC
GCTAGCACTCTGGTATCACGGTACCTTTGTGCGCCTGTTTTATACCCCCTCCCCCAACTGTAACTTAGAAGTAACA
CACACCGATCAACAGTCAGCGTGGCACACCAGCCACGTTTTGATCAAGCACTTCTGTTACCCCGGACTGAGTATCA
ATAGACTGCTCACGCGGTTGAAGGAGAAAGCGTTCGTTATCCGGCCAACTACTTCGAAAAACCTAGTAACACCGTG
GAAGTTGCAGAGTGTTTCGCTCAGCACTACCCCAGTGTAGATCAGGTCGATGAGTCACCGCATTCCCCACGGGCGA
CCGTGGCGGTGGCTGCGTTGGCGGCCTGCCCATGGGGAAACCCATGGGACGCTCTAATACAGACATGGTGCGAAGA
GTCTATTGAGCTAGTTGGTAGTCCTCCGGCCCCTGAATGCGGCTAATCCTAACTGCGGAGCACACACCCTCAAGCC
AGAGGGCAGTGTGTCGTAACGGGCAACTCTGCAGCGGAACCGACTACTTTGGGTGTCCGTGTTTCATTTTATTCCT
ATACTGGCTGCTTATGGTGACAATTGAGAGATCGTTACCATATAGCTATTGGATTGGCCATCCGGTGACTAATAGA
GCTATTATATATCCCTTTGTTGGGTTTATACCACTTAGCTTGAAAGAGGTTAAAACATTACAATTCATTGTTAAGT
TGAATACAGCAAAATGGCTCTGCCTGTGACAGCTCTGCTGCTGCCTCTGGCTCTGCTTCTGCATGCCGCCAGACCT
GACATCCAGATGACCCAGACAACCAGCAGCCTGTCTGCCAGCCTGGGCGACAGAGTGACCATCAGCTGTAGAGCCA
GCCAGGACATCAGCAAGTACCTGAACTGGTATCAGCAGAAACCCGACGGCACCGTGAAGCTGCTGATCTACCACAC
CAGCAGACTGCACAGCGGCGTGCCATCTAGATTTTCTGGCAGCGGCTCTGGCACCGACTACAGCCTGACAATCAGC
AACCTGGAACAAGAGGATATCGCTACCTACTTCTGCCAGCAAGGCAACACCCTGCCTTACACATTCGGCGGAGGCA
CCAAGCTGGAAATCACAGGTGGCGGAGGATCTGGCGGAGGTGGAAGCGGAGGCGGTGGATCTGAAGTGAAACTGCA
AGAGTCTGGCCCTGGCCTGGTGGCTCCATCTCAGTCTCTGAGCGTGACCTGTACCGTCAGCGGAGTGTCTCTGCCT
GATTACGGCGTGTCCTGGATCAGACAGCCTCCTAGAAAAGGCCTGGAATGGCTGGGCGTGATCTGGGGCAGCGAGA
CAACCTACTACAACAGCGCCCTGAAGTCCAGGCTGACCATCATCAAGGACAACTCCAAGAGCCAGGTGTTCCTGAA
GATGAACAGCCTGCAGACCGACGACACCGCCATCTACTACTGCGCCAAGCACTACTACTACGGCGGCAGCTACGCC
ATGGATTACTGGGGCCAGGGAACCAGCGTGACCGTGTCTAGCACAACAACCCCTGCTCCTAGACCTCCTACACCAG
CTCCTACAATCGCCAGCCAGCCTCTGTCTCTGAGGCCAGAGGCTTGTAGACCTGCTGCTGGCGGAGCTGTGCACAC
AAGAGGCCTGGATTTCGCCTGCGACATCTACATCTGGGCCCCTCTGGCTGGAACATGTGGCGTGCTGCTGCTGAGC
CTGGTCATCACCCTGTATTGCAAGAGAGGCCGGAAGAAGCTGCTCTACATCTTCAAGCAGCCCTTCATGAGGCCCG
TGCAGACCACACAAGAAGAGGACGGCTGCTCCTGCAGATTCCCCGAGGAAGAGGAAGGCGGCTGCGAGCTGAGAGT
GAAGTTCAGCAGAAGCGCTGACGCCCCTGCTTACCAGCAGGGACAGAACCAGCTGTACAACGAGCTGAACCTGGGG
AGAAGAGAAGAGTACGACGTGCTGGACAAGCGGAGAGGCAGAGATCCTGAGATGGGCGGCAAGCCCAGAAGAAAGA
ACCCTCAAGAGGGCCTGTATAATGAGCTGCAGAAAGACAAGATGGCCGAGGCCTACAGCGAGATCGGAATGAAGGG
CGAGCGCAGAAGAGGCAAGGGACACGATGGACTGTACCAGGGCCTGAGCACAGCCACCAAGGACACATATGACGCC
CTGCACATGCAGGCCCTGCCTCCAAGATAATAGAAAAACAAAAAACAAAAACCACACAAATGGTCGCCGAAACTAC
TGAAAGCAT (SEQ ID NO: 217)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCCCCCCTTTTCCCTAACCCCAACCGCCGTATCTGGTGGCGGCAAG
ACACACGGGTCTTTCCCTCTAAAGCACAAATGTGTGTGTGTCCCAGGTCCTCCTGCGCTCGGTGCGGGAGTGCTCC
CACCCAACTGTTGTAAGCCTGTCCAACGTGTCGTCCTGGCAAGACTATGACGTCGCATGTTCCGCTGCGGATGCCG
ACCGGGTAACCGGTTCCCCAGTGTGTGTAGTGCGATCTTCCAGGTTCCCCTGGTTGGCGTTGTCCAGAAACTGCTT
CAGGTAAGTGGGGTGTGCCCAATCCCTGCAAAGGTTGATTCTCTCACCACCTTAGGAATGCTCCGGAGGTACCCCA
GCAACAGCTGGGATCTGACCGGAGGCTAATTGTCTACGGGTGGTGTTTCCTTTTTCTTTTTCCACAACTTCATTGC
TGACAACTCACTGACTACCCATTTGCTCTCTTGTGCCTTTCTGCTCTGGTTCAAGCTCCTTGATTGTTTTTGATTG
CTTTCTATTGCTTTCTTCCACTCAACCCTTGCTCAGTTCAAAGTCATGGCTCTGCCTGTGACAGCTCTGCTGCTGC
CTCTGGCTCTGCTTCTGCATGCCGCCAGACCTGACATCCAGATGACCCAGACAACCAGCAGCCTGTCTGCCAGCCT
GGGCGACAGAGTGACCATCAGCTGTAGAGCCAGCCAGGACATCAGCAAGTACCTGAACTGGTATCAGCAGAAACCC
GACGGCACCGTGAAGCTGCTGATCTACCACACCAGCAGACTGCACAGCGGCGTGCCATCTAGATTTTCTGGCAGCG
GCTCTGGCACCGACTACAGCCTGACAATCAGCAACCTGGAACAAGAGGATATCGCTACCTACTTCTGCCAGCAAGG
CAACACCCTGCCTTACACATTCGGCGGAGGCACCAAGCTGGAAATCACAGGTGGCGGAGGATCTGGCGGAGGTGGA
AGCGGAGGCGGTGGATCTGAAGTGAAACTGCAAGAGTCTGGCCCTGGCCTGGTGGCTCCATCTCAGTCTCTGAGCG
TGACCTGTACCGTCAGCGGAGTGTCTCTGCCTGATTACGGCGTGTCCTGGATCAGACAGCCTCCTAGAAAAGGCCT
GGAATGGCTGGGCGTGATCTGGGGCAGCGAGACAACCTACTACAACAGCGCCCTGAAGTCCAGGCTGACCATCATC
AAGGACAACTCCAAGAGCCAGGTGTTCCTGAAGATGAACAGCCTGCAGACCGACGACACCGCCATCTACTACTGCG
CCAAGCACTACTACTACGGCGGCAGCTACGCCATGGATTACTGGGGCCAGGGAACCAGCGTGACCGTGTCTAGCAC
AACAACCCCTGCTCCTAGACCTCCTACACCAGCTCCTACAATCGCCAGCCAGCCTCTGTCTCTGAGGCCAGAGGCT
TGTAGACCTGCTGCTGGCGGAGCTGTGCACACAAGAGGCCTGGATTTCGCCTGCGACATCTACATCTGGGCCCCTC
TGGCTGGAACATGTGGCGTGCTGCTGCTGAGCCTGGTCATCACCCTGTATTGCAAGAGAGGCCGGAAGAAGCTGCT
CTACATCTTCAAGCAGCCCTTCATGAGGCCCGTGCAGACCACACAAGAAGAGGACGGCTGCTCCTGCAGATTCCCC
GAGGAAGAGGAAGGCGGCTGCGAGCTGAGAGTGAAGTTCAGCAGAAGCGCTGACGCCCCTGCTTACCAGCAGGGAC
AGAACCAGCTGTACAACGAGCTGAACCTGGGGAGAAGAGAAGAGTACGACGTGCTGGACAAGCGGAGAGGCAGAGA
TCCTGAGATGGGCGGCAAGCCCAGAAGAAAGAACCCTCAAGAGGGCCTGTATAATGAGCTGCAGAAAGACAAGATG
GCCGAGGCCTACAGCGAGATCGGAATGAAGGGCGAGCGCAGAAGAGGCAAGGGACACGATGGACTGTACCAGGGCC
TGAGCACAGCCACCAAGGACACATATGACGCCCTGCACATGCAGGCCCTGCCTCCAAGATAATAGAAAAACAAAAA
ACAAAAACCACACAAATGGTCGCCGAAACTACTGAAAGCAT (SEQ ID NO: 218)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTTAAGTGTTGTGCCCAATCTCTTGACTCCTGCTGGAACCACCG
ACCAGTAGTGTCCAAAATGCCAGGTGGAAAATCCTCCCTTCCCCTCTGGGCTTCATGCCCGGCATCCTCCCCCCAG
CCTGACGTGCCACAGGCTGTGCAAAGACCCCGCGAAAGCTGCCAAAAGTGGCAATTGTGGGTCCCCCCTTTGTCAA
GGCGTCGAGTCTTTCTCCCTTAAGGCTAGTCCTGTCAGTGAACTCTGTCGGGCAACTAGTGACGCCACTGCATGCC
TCCGACCTCGGCCGCGGAGTGCTGCCCCCCAAGTCATGCCCCTGACCACAAGTTGTGCTGTCTGGCAAACATTGTC
TGTGAGAATGTTCCGCTGTGGCTGCCAAGCCTGGTAACAGGCTGCCCCAGTGTGCGTAATTCTCATCCAGACTTCG
GTCTGGCAACTTGCTGTTAAGACATGGCGTAAGGGGCGTGTGCCAACGCCCTGGAACGAGTGTCCACTCTAATACC
CCGAGGAATGCTACGCAGGTACCCCTGGCTCGCCAGGGATCTGAGCGTAGGCTAATTGTCTAAGGGTATTTTCATT
TCCCACCCTCTTCTTCTTGTTCATAATGGCTCTGCCTGTGACAGCTCTGCTGCTGCCTCTGGCTCTGCTTCTGCAT
GCCGCCAGACCTGACATCCAGATGACCCAGACAACCAGCAGCCTGTCTGCCAGCCTGGGCGACAGAGTGACCATCA
GCTGTAGAGCCAGCCAGGACATCAGCAAGTACCTGAACTGGTATCAGCAGAAACCCGACGGCACCGTGAAGCTGCT
GATCTACCACACCAGCAGACTGCACAGCGGCGTGCCATCTAGATTTTCTGGCAGCGGCTCTGGCACCGACTACAGC
CTGACAATCAGCAACCTGGAACAAGAGGATATCGCTACCTACTTCTGCCAGCAAGGCAACACCCTGCCTTACACAT
TCGGCGGAGGCACCAAGCTGGAAATCACAGGTGGCGGAGGATCTGGCGGAGGTGGAAGCGGAGGCGGTGGATCTGA
AGTGAAACTGCAAGAGTCTGGCCCTGGCCTGGTGGCTCCATCTCAGTCTCTGAGCGTGACCTGTACCGTCAGCGGA
GTGTCTCTGCCTGATTACGGCGTGTCCTGGATCAGACAGCCTCCTAGAAAAGGCCTGGAATGGCTGGGCGTGATCT
GGGGCAGCGAGACAACCTACTACAACAGCGCCCTGAAGTCCAGGCTGACCATCATCAAGGACAACTCCAAGAGCCA
GGTGTTCCTGAAGATGAACAGCCTGCAGACCGACGACACCGCCATCTACTACTGCGCCAAGCACTACTACTACGGC
GGCAGCTACGCCATGGATTACTGGGGCCAGGGAACCAGCGTGACCGTGTCTAGCACAACAACCCCTGCTCCTAGAC
CTCCTACACCAGCTCCTACAATCGCCAGCCAGCCTCTGTCTCTGAGGCCAGAGGCTTGTAGACCTGCTGCTGGCGG
AGCTGTGCACACAAGAGGCCTGGATTTCGCCTGCGACATCTACATCTGGGCCCCTCTGGCTGGAACATGTGGCGTG
CTGCTGCTGAGCCTGGTCATCACCCTGTATTGCAAGAGAGGCCGGAAGAAGCTGCTCTACATCTTCAAGCAGCCCT
TCATGAGGCCCGTGCAGACCACACAAGAAGAGGACGGCTGCTCCTGCAGATTCCCCGAGGAAGAGGAAGGCGGCTG
CGAGCTGAGAGTGAAGTTCAGCAGAAGCGCTGACGCCCCTGCTTACCAGCAGGGACAGAACCAGCTGTACAACGAG
CTGAACCTGGGGAGAAGAGAAGAGTACGACGTGCTGGACAAGCGGAGAGGCAGAGATCCTGAGATGGGCGGCAAGC
CCAGAAGAAAGAACCCTCAAGAGGGCCTGTATAATGAGCTGCAGAAAGACAAGATGGCCGAGGCCTACAGCGAGAT
CGGAATGAAGGGCGAGCGCAGAAGAGGCAAGGGACACGATGGACTGTACCAGGGCCTGAGCACAGCCACCAAGGAC
ACATATGACGCCCTGCACATGCAGGCCCTGCCTCCAAGATAATAGAAAAACAAAAAACAAAAACCACACAAATGGT
CGCCGAAACTACTGAAAGCAT (SEQ ID NO: 219)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTTCCCCTGTTCGTAACTAAGTGTGTGCCCAATCTCCTCACTCC
TGCTGGCTTCACCGACCGGCAGTGTCCAAAATGETAGGTGAATCCCCTCCCTTTCCTCTGGGCTTCTGCCCAGCTT
CCTCCCCCCAGCCTGACGTGACACAGGCTGTGCAAAGACCCCGCGAAAGCTGCCAAAAGTGGCAATTGTGGGTCCC
CCCTTTGTAAAGGCGTCGAGTCTTTCTCCCTCAAGGCTAGACCCGTCAGTGAATTCTGTCGGGCAACTAGTGACGC
CACTGCACGCCTCTGACCTCGGCCGCGGAGTGCTGCCCCCCAAGTCGTGCCCCTGACCACAAGTTGTGCTGTCTGG
CAAACATTGTCTGTGAGAATGTTCCGCTGTGGCTGCCAAGCCTGGCAACAGGCTGCCCCAGTGTGCGTAGTTCTCA
TCCAGACTTCGGTCTGGCAACTTGCTGTTAAGACACGGCGTAAGGGGCGTGTGCCAACGCCCTGGAACGAGTGTCC
ACTCTAATACCCCGAGGAATGCTACGCAGGTACCCCTGGTTCGCCAGGGATCTGAGCGTAGGCTAATTGTCTAAGG
GTATTTTCATTTCCCATTCTTTCTTTCTTGTTCATAATGGCTCTGCCTGTGACAGCTCTGCTGCTGCCTCTGGCTC
TGCTTCTGCATGCCGCCAGACCTGACATCCAGATGACCCAGACAACCAGCAGCCTGTCTGCCAGCCTGGGCGACAG
AGTGACCATCAGCTGTAGAGCCAGCCAGGACATCAGCAAGTACCTGAACTGGTATCAGCAGAAACCCGACGGCACC
GTGAAGCTGCTGATCTACCACACCAGCAGACTGCACAGCGGCGTGCCATCTAGATTTTCTGGCAGCGGCTCTGGCA
CCGACTACAGCCTGACAATCAGCAACCTGGAACAAGAGGATATCGCTACCTACTTCTGCCAGCAAGGCAACACCCT
GCCTTACACATTCGGCGGAGGCACCAAGCTGGAAATCACAGGTGGCGGAGGATCTGGCGGAGGTGGAAGCGGAGGC
GGTGGATCTGAAGTGAAACTGCAAGAGTCTGGCCCTGGCCTGGTGGCTCCATCTCAGTCTCTGAGCGTGACCTGTA
CCGTCAGCGGAGTGTCTCTGCCTGATTACGGCGTGTCCTGGATCAGACAGCCTCCTAGAAAAGGCCTGGAATGGCT
GGGCGTGATCTGGGGCAGCGAGACAACCTACTACAACAGCGCCCTGAAGTCCAGGCTGACCATCATCAAGGACAAC
TCCAAGAGCCAGGTGTTCCTGAAGATGAACAGCCTGCAGACCGACGACACCGCCATCTACTACTGCGCCAAGCACT
ACTACTACGGCGGCAGCTACGCCATGGATTACTGGGGCCAGGGAACCAGCGTGACCGTGTCTAGCACAACAACCCC
TGCTCCTAGACCTCCTACACCAGCTCCTACAATCGCCAGCCAGCCTCTGTCTCTGAGGCCAGAGGCTTGTAGACCT
GCTGCTGGCGGAGCTGTGCACACAAGAGGCCTGGATTTCGCCTGCGACATCTACATCTGGGCCCCTCTGGCTGGAA
CATGTGGCGTGCTGCTGCTGAGCCTGGTCATCACCCTGTATTGCAAGAGAGGCCGGAAGAAGCTGCTCTACATCTT
CAAGCAGCCCTTCATGAGGCCCGTGCAGACCACACAAGAAGAGGACGGCTGCTCCTGCAGATTCCCCGAGGAAGAG
GAAGGCGGCTGCGAGCTGAGAGTGAAGTTCAGCAGAAGCGCTGACGCCCCTGCTTACCAGCAGGGACAGAACCAGC
TGTACAACGAGCTGAACCTGGGGAGAAGAGAAGAGTACGACGTGCTGGACAAGCGGAGAGGCAGAGATCCTGAGAT
GGGCGGCAAGCCCAGAAGAAAGAACCCTCAAGAGGGCCTGTATAATGAGCTGCAGAAAGACAAGATGGCCGAGGCC
TACAGCGAGATCGGAATGAAGGGCGAGCGCAGAAGAGGCAAGGGACACGATGGACTGTACCAGGGCCTGAGCACAG
CCACCAAGGACACATATGACGCCCTGCACATGCAGGCCCTGCCTCCAAGATAATAGAAAAACAAAAAACAAAAACC
ACACAAATGGTCGCCGAAACTACTGAAAGCAT (SEQ ID NO: 220)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCTAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAAGAGAGGCCGGAAGAAGCTGCTCTACATCTTCAAGCAGCCCTTC
ATGAGGCCCGTGCAGACCACACAAGAAGAGGACGGCTGCTCCTGCAGATTCCCCGAGGAAGAGGAAGGCGGCTGCG
AGCTGAGAGTGAAGTTCAGCAGAAGCGCTGACGCCCCTGCTTACCAGCAGGGACAGAACCAGCTGTACAACGAGCT
GAACCTGGGGAGAAGAGAAGAGTACGACGTGCTGGACAAGCGGAGAGGCAGAGATCCTGAGATGGGCGGCAAGCCC
AGAAGAAAGAACCCTCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTACCAGGGCCTGAGCACAGCCACCAAGGACAC
ATATGACGCCCTGCACATGCAGGCCCTGCCTCCAAGATAATAGACAACAACAAACAACAAAACAACAAACAACACA
AAACCAAACCACACAAATGGTCGCCGAAACTACTGAAAGCAT (SEQ ID NO: 221)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTCAAGTGTGTGTGCTCGTAATCTTGACTCCTGCCGGAATGCCG
CCCGGTTCAGTGAACAAACAGCTAGGCAAGTCCCTCCCTTCCCCTGTGGTCGGTTCTCACCGGCCACCATCCCTCC
CCCAGCCTGACGTGTTACAGGCTGTGCAAAGCCCCCGCGAAAGCTGCTCACGTGGCAATTGTGGGTCCCCCCTTTG
TCAAGACACCGAGTCTTTCTCCCTTAAGGCTAGCCCGGTCCCACGAACGTGGAACTGGCAACTAGTGGTGTCACTA
CACGCCTCCGACCTCGGACGCGGAGTGCTGTTCCCCAAGCTGTAACCCTGACCCAAGACTGTGCTGCCTGGCAAGC
ACCGTCTGGGAAGATGTTCCGCTGTGGCTGCCAAACCTGGTAACAGGTGCCCCAGTGTGTGTAGTCTTCCTCCAGT
CTCCGGACTGGCAGTCTTGTGTAAAGATGCAGTGTAAGGTTCAAGTGCCAAATCCCTGGAAGGAGTGACCCTCTAC
TGCCCTAGGAATGCTGTGCAGGTACCCCCAACTTCGGTTGGGGATCTGAGCACAGGCTAATTGTCTACGGGTAGTT
TCATTTCCCATCCTCTCTTTTTTGGCATCATGGCTCTGCCTGTGACAGCTCTGCTGCTGCCTCTGGCTCTGCTTCT
GCATGCCGCCAGACCTGACATCCAGATGACCCAGACAACCAGCAGCCTGTCTGCCAGCCTGGGCGACAGAGTGACC
ATCAGCTGTAGAGCCAGCCAGGACATCAGCAAGTACCTGAACTGGTATCAGCAGAAACCCGACGGCACCGTGAAGC
TGCTGATCTACCACACCAGCAGACTGCACAGCGGCGTGCCATCTAGATTTTCTGGCAGCGGCTCTGGCACCGACTA
CAGCCTGACAATCAGCAACCTGGAACAAGAGGATATCGCTACCTACTTCTGCCAGCAAGGCAACACCCTGCCTTAC
ACATTCGGCGGAGGCACCAAGCTGGAAATCACAGGTGGCGGAGGATCTGGCGGAGGTGGAAGCGGAGGCGGTGGAT
CTGAAGTGAAACTGCAAGAGTCTGGCCCTGGCCTGGTGGCTCCATCTCAGTCTCTGAGCGTGACCTGTACCGTCAG
CGGAGTGTCTCTGCCTGATTACGGCGTGTCCTGGATCAGACAGCCTCCTAGAAAAGGCCTGGAATGGCTGGGCGTG
ATCTGGGGCAGCGAGACAACCTACTACAACAGCGCCCTGAAGTCCAGGCTGACCATCATCAAGGACAACTCCAAGA
GCCAGGTGTTCCTGAAGATGAACAGCCTGCAGACCGACGACACCGCCATCTACTACTGCGCCAAGCACTACTACTA
CGGCGGCAGCTACGCCATGGATTACTGGGGCCAGGGAACCAGCGTGACCGTGTCTAGCACAACAACCCCTGCTCCT
AGACCTCCTACACCAGCTCCTACAATCGCCAGCCAGCCTCTGTCTCTGAGGCCAGAGGCTTGTAGACCTGCTGCTG
GCGGAGCTGTGCACACAAGAGGCCTGGATTTCGCCTGCGACATCTACATCTGGGCCCCTCTGGCTGGAACATGTGG
CGTGCTGCTGCTGAGCCTGGTCATCACCCTGTATTGCAAGAGAGGCCGGAAGAAGCTGCTCTACATCTTCAAGCAG
CCCTTCATGAGGCCCGTGCAGACCACACAAGAAGAGGACGGCTGCTCCTGCAGATTCCCCGAGGAAGAGGAAGGCG
GCTGCGAGCTGAGAGTGAAGTTCAGCAGAAGCGCTGACGCCCCTGCTTACCAGCAGGGACAGAACCAGCTGTACAA
CGAGCTGAACCTGGGGAGAAGAGAAGAGTACGACGTGCTGGACAAGCGGAGAGGCAGAGATCCTGAGATGGGCGGC
AAGCCCAGAAGAAAGAACCCTCAAGAGGGCCTGTATAATGAGCTGCAGAAAGACAAGATGGCCGAGGCCTACAGCG
AGATCGGAATGAAGGGCGAGCGCAGAAGAGGCAAGGGACACGATGGACTGTACCAGGGCCTGAGCACAGCCACCAA
GGACACATATGACGCCCTGCACATGCAGGCCCTGCCTCCAAGATAATAGAAAAACAAAAAACAAAAACCACACAAA
TGGTCGCCGAAACTACTGAAAGCAT (SEQ ID NO: 222)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTGTAGGTAGAAGGTATAAACGACCAGCCACACACTTCCTACCCA
AAGCGCCAATAACAGGAGCCCGGATTGGTCTGGCACGCAAAGGAGTAGACATCCCCCAATGTTTTCCGAACCAAGC
GGCAGCAGCGTCGGTTAGGATCCTCATAATTCTGATCGAAGGACAGTACACGCGCTACTCAGGGTCTTCTATGATG
ATTACGATTTACTTACGACGGTGTGATGTAACGCCCTCGGCGACTTACTTGGACCTGTTCCCGAATTGAGAATGTG
TAGGCCAGCGACCAATCGCCACCACGAGGCCTAGACTGATCAACTTCAACTGTGAGCGACTCGCATCTGAGGAGTT
GACCTAGCTAACACTTCCCATCCACTCTAATTTGGCTAACAAGGCTGCCACGTGATGATCCTTCTTCTAGGGTGTC
TACCTGAGTAAGGGTACCCTTCTCGGAATAACGATAAGCGAAGGAGGACATGAACCTAGGCTGGATGTGATTAGCT
CCGTAAGCTGCCAACCTGGAGCGCGTCAGATTCGGCCAACGGCAGATTGGGCTGATCGGGAGTGAGGAATCCCTGC
TTACGCTCACTGGGTGATTCCACGTAAAATGTCCAGTTATTATCTGTGTCCTCTTAACACATTCTTCTGTTTAAGC
TTTTCGCCCGTTCTATAAACCTTGTCTGACTAAGTCCTTAGTCCGCTGCCATTACCGATTGGGTTGCGTAGGCAGC
TTCTATTGAAACGATGGCTCTGCCTGTGACAGCTCTGCTGCTGCCTCTGGCTCTGCTTCTGCATGCCGCCAGACCT
GACATCCAGATGACCCAGACAACCAGCAGCCTGTCTGCCAGCCTGGGCGACAGAGTGACCATCAGCTGTAGAGCCA
GCCAGGACATCAGCAAGTACCTGAACTGGTATCAGCAGAAACCCGACGGCACCGTGAAGCTGCTGATCTACCACAC
CAGCAGACTGCACAGCGGCGTGCCATCTAGATTTTCTGGCAGCGGCTCTGGCACCGACTACAGCCTGACAATCAGC
AACCTGGAACAAGAGGATATCGCTACCTACTTCTGCCAGCAAGGCAACACCCTGCCTTACACATTCGGCGGAGGCA
CCAAGCTGGAAATCACAGGTGGCGGAGGATCTGGCGGAGGTGGAAGCGGAGGCGGTGGATCTGAAGTGAAACTGCA
AGAGTCTGGCCCTGGCCTGGTGGCTCCATCTCAGTCTCTGAGCGTGACCTGTACCGTCAGCGGAGTGTCTCTGCCT
GATTACGGCGTGTCCTGGATCAGACAGCCTCCTAGAAAAGGCCTGGAATGGCTGGGCGTGATCTGGGGCAGCGAGA
CAACCTACTACAACAGCGCCCTGAAGTCCAGGCTGACCATCATCAAGGACAACTCCAAGAGCCAGGTGTTCCTGAA
GATGAACAGCCTGCAGACCGACGACACCGCCATCTACTACTGCGCCAAGCACTACTACTACGGCGGCAGCTACGCC
ATGGATTACTGGGGCCAGGGAACCAGCGTGACCGTGTCTAGCACAACAACCCCTGCTCCTAGACCTCCTACACCAG
CTCCTACAATCGCCAGCCAGCCTCTGTCTCTGAGGCCAGAGGCTTGTAGACCTGCTGCTGGCGGAGCTGTGCACAC
AAGAGGCCTGGATTTCGCCTGCGACATCTACATCTGGGCCCCTCTGGCTGGAACATGTGGCGTGCTGCTGCTGAGC
CTGGTCATCACCCTGTATTGCAAGAGAGGCCGGAAGAAGCTGCTCTACATCTTCAAGCAGCCCTTCATGAGGCCCG
TGCAGACCACACAAGAAGAGGACGGCTGCTCCTGCAGATTCCCCGAGGAAGAGGAAGGCGGCTGCGAGCTGAGAGT
GAAGTTCAGCAGAAGCGCTGACGCCCCTGCTTACCAGCAGGGACAGAACCAGCTGTACAACGAGCTGAACCTGGGG
AGAAGAGAAGAGTACGACGTGCTGGACAAGCGGAGAGGCAGAGATCCTGAGATGGGCGGCAAGCCCAGAAGAAAGA
ACCCTCAAGAGGGCCTGTATAATGAGCTGCAGAAAGACAAGATGGCCGAGGCCTACAGCGAGATCGGAATGAAGGG
CGAGCGCAGAAGAGGCAAGGGACACGATGGACTGTACCAGGGCCTGAGCACAGCCACCAAGGACACATATGACGCC
CTGCACATGCAGGCCCTGCCTCCAAGATAATAGAAAAACAAAAAACAAAAACCACACAAATGGTCGCCGAAACTAC
TGAAAGCAT (SEQ ID NO: 223)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCGCCCGTCCCCCTCACCCTCTTTTCCGGTGGCCACGCCCGGGCCA
CCGATACTTCCCTTCACTCCCTCGGGACTGTTGGGGAGGAACACAACAGGGCTCCCCTGTATTTCCTCTTCCCATT
CCCCCTTTCCTAACCCCAACCGCCGTATCTGGTGGCGGTAAGACACACGGGTCTTTCCCTCTAAAGCACAATTGTG
TGTGTGTCCCAGGTCCTCCTGCGTTCGGTGCGGGAGTGCTCCCACCCAACTGTTGTAAGCCTGTCCAACGTGTCGT
CCTGGCAAGACTATGACGTCGCATGTTCCGCTGTGGATGCCGACCGGGTAACCGGTTCCCCAGTGTGTGTAGTGCG
ATCTTCCAGGTTCTCCTGGTTGGCGTTGTCCAGAAACTGCTTCGGGTAAGTGGGGTGTGCCCAATCCCTACAAGGG
TTGATTCTTTCACCACCTTAGGAATGCTCCGGAGGTACCCCAGCAACAGCTGGGATCTGACCGGAGGCTAATTGTC
TACGGGTGGTGTTTCCATTTTCTTTTTCACACAACTTCATTGCTGACAACTCACTGACTAATCACTTGCTCTCTTG
TGCCTTTCTGCTCTGGTTCAAGTTCCTTGATTGTTTGTTTGATTGCTTTTCACTGCTTTCTTCCCACAATCCTTGC
TCAGTTCAAAGTCATGGCTCTGCCTGTGACAGCTCTGCTGCTGCCTCTGGCTCTGCTTCTGCATGCCGCCAGACCT
GACATCCAGATGACCCAGACAACCAGCAGCCTGTCTGCCAGCCTGGGCGACAGAGTGACCATCAGCTGTAGAGCCA
GCCAGGACATCAGCAAGTACCTGAACTGGTATCAGCAGAAACCCGACGGCACCGTGAAGCTGCTGATCTACCACAC
CAGCAGACTGCACAGCGGCGTGCCATCTAGATTTTCTGGCAGCGGCTCTGGCACCGACTACAGCCTGACAATCAGC
AACCTGGAACAAGAGGATATCGCTACCTACTTCTGCCAGCAAGGCAACACCCTGCCTTACACATTCGGCGGAGGCA
CCAAGCTGGAAATCACAGGTGGCGGAGGATCTGGCGGAGGTGGAAGCGGAGGCGGTGGATCTGAAGTGAAACTGCA
AGAGTCTGGCCCTGGCCTGGTGGCTCCATCTCAGTCTCTGAGCGTGACCTGTACCGTCAGCGGAGTGTCTCTGCCT
GATTACGGCGTGTCCTGGATCAGACAGCCTCCTAGAAAAGGCCTGGAATGGCTGGGCGTGATCTGGGGCAGCGAGA
CAACCTACTACAACAGCGCCCTGAAGTCCAGGCTGACCATCATCAAGGACAACTCCAAGAGCCAGGTGTTCCTGAA
GATGAACAGCCTGCAGACCGACGACACCGCCATCTACTACTGCGCCAAGCACTACTACTACGGCGGCAGCTACGCC
ATGGATTACTGGGGCCAGGGAACCAGCGTGACCGTGTCTAGCACAACAACCCCTGCTCCTAGACCTCCTACACCAG
CTCCTACAATCGCCAGCCAGCCTCTGTCTCTGAGGCCAGAGGCTTGTAGACCTGCTGCTGGCGGAGCTGTGCACAC
AAGAGGCCTGGATTTCGCCTGCGACATCTACATCTGGGCCCCTCTGGCTGGAACATGTGGCGTGCTGCTGCTGAGC
CTGGTCATCACCCTGTATTGCAAGAGAGGCCGGAAGAAGCTGCTCTACATCTTCAAGCAGCCCTTCATGAGGCCCG
TGCAGACCACACAAGAAGAGGACGGCTGCTCCTGCAGATTCCCCGAGGAAGAGGAAGGCGGCTGCGAGCTGAGAGT
GAAGTTCAGCAGAAGCGCTGACGCCCCTGCTTACCAGCAGGGACAGAACCAGCTGTACAACGAGCTGAACCTGGGG
AGAAGAGAAGAGTACGACGTGCTGGACAAGCGGAGAGGCAGAGATCCTGAGATGGGCGGCAAGCCCAGAAGAAAGA
ACCCTCAAGAGGGCCTGTATAATGAGCTGCAGAAAGACAAGATGGCCGAGGCCTACAGCGAGATCGGAATGAAGGG
CGAGCGCAGAAGAGGCAAGGGACACGATGGACTGTACCAGGGCCTGAGCACAGCCACCAAGGACACATATGACGCC
CTGCACATGCAGGCCCTGCCTCCAAGATAATAGAAAAACAAAAAACAAAAACCACACAAATGGTCGCCGAAACTAC
TGAAAGCAT (SEQ ID NO: 224)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTCCCCCTTTTCCCAACCCCAACCGCCGTATCTGGTGGCGGCAA
GACACACGGGTCTTTCCCTCTAAAGCACAATTGTGTGTGTGTCCCAGGTCCTCCTGCGTACGGTGCGGGAGTGCTC
CCACCCAACTGTTGTAAGCCTGTCCAACGCGTCGTCCTGGCAAGACTATGACGTCGCATGTTCCGCTGCGGATGCC
GACCGGGTAACCGGTTCCCCAGTGTGTGTAGTGCGATCTTCCAGGTCCTCCTGGTTGGCGTTGTCCAGAAACTGCT
TCAGGTAAGTGGGGTGTGCCCAATCCCTACAAAGGTTGATTCTTTCACCACCTTAGGAATGCTCCGGAGGTACCCC
AGCAACAGCTGGGATCTGACCGGAGGCTAATTGTCTACGGGTGGTGTTTCCTTTTTCTTTTCACACAACTCTACTG
CTGACAACTCACTGACTATCCACTTGCTCTCTTGTGCCTTTCTGCTCTGGTTCAAGTTCCTTGATTGTTTTTGACT
GCTTTTCACTGCTTTTCTTCTCACAATCCTTGCTCAGTTCAAAGTCATGGCTCTGCCTGTGACAGCTCTGCTGCTG
CCTCTGGCTCTGCTTCTGCATGCCGCCAGACCTGACATCCAGATGACCCAGACAACCAGCAGCCTGTCTGCCAGCC
TGGGCGACAGAGTGACCATCAGCTGTAGAGCCAGCCAGGACATCAGCAAGTACCTGAACTGGTATCAGCAGAAACC
CGACGGCACCGTGAAGCTGCTGATCTACCACACCAGCAGACTGCACAGCGGCGTGCCATCTAGATTTTCTGGCAGC
GGCTCTGGCACCGACTACAGCCTGACAATCAGCAACCTGGAACAAGAGGATATCGCTACCTACTTCTGCCAGCAAG
GCAACACCCTGCCTTACACATTCGGCGGAGGCACCAAGCTGGAAATCACAGGTGGCGGAGGATCTGGCGGAGGTGG
AAGCGGAGGCGGTGGATCTGAAGTGAAACTGCAAGAGTCTGGCCCTGGCCTGGTGGCTCCATCTCAGTCTCTGAGC
GTGACCTGTACCGTCAGCGGAGTGTCTCTGCCTGATTACGGCGTGTCCTGGATCAGACAGCCTCCTAGAAAAGGCC
TGGAATGGCTGGGCGTGATCTGGGGCAGCGAGACAACCTACTACAACAGCGCCCTGAAGTCCAGGCTGACCATCAT
CAAGGACAACTCCAAGAGCCAGGTGTTCCTGAAGATGAACAGCCTGCAGACCGACGACACCGCCATCTACTACTGC
GCCAAGCACTACTACTACGGCGGCAGCTACGCCATGGATTACTGGGGCCAGGGAACCAGCGTGACCGTGTCTAGCA
CAACAACCCCTGCTCCTAGACCTCCTACACCAGCTCCTACAATCGCCAGCCAGCCTCTGTCTCTGAGGCCAGAGGC
TTGTAGACCTGCTGCTGGCGGAGCTGTGCACACAAGAGGCCTGGATTTCGCCTGCGACATCTACATCTGGGCCCCT
CTGGCTGGAACATGTGGCGTGCTGCTGCTGAGCCTGGTCATCACCCTGTATTGCAAGAGAGGCCGGAAGAAGCTGC
TCTACATCTTCAAGCAGCCCTTCATGAGGCCCGTGCAGACCACACAAGAAGAGGACGGCTGCTCCTGCAGATTCCC
CGAGGAAGAGGAAGGCGGCTGCGAGCTGAGAGTGAAGTTCAGCAGAAGCGCTGACGCCCCTGCTTACCAGCAGGGA
CAGAACCAGCTGTACAACGAGCTGAACCTGGGGAGAAGAGAAGAGTACGACGTGCTGGACAAGCGGAGAGGCAGAG
ATCCTGAGATGGGCGGCAAGCCCAGAAGAAAGAACCCTCAAGAGGGCCTGTATAATGAGCTGCAGAAAGACAAGAT
GGCCGAGGCCTACAGCGAGATCGGAATGAAGGGCGAGCGCAGAAGAGGCAAGGGACACGATGGACTGTACCAGGGC
CTGAGCACAGCCACCAAGGACACATATGACGCCCTGCACATGCAGGCCCTGCCTCCAAGATAATAGAAAAACAAAA
AACAAAAACCACACAAATGGTCGCCGAAACTACTGAAAGCAT (SEQ ID NO: 225)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCGTAACTTCAAGTGTGTGTGCTCGTAATCTTGACTCCTGCCGGAA
TGCCGCCCGGTTCAGTGAACAAACAGCTAGGCAAGTCCCTCCCTTCCCCTGTGGTCGGTTCTCACCGGCCACCATC
CCTCCCCCAGCCTGACGTGTTACAGGCTGTGCAAAGCCCCCGCGAAAGCTGCTCACGTGGCAATTGTGGGTCCCCC
CTTTGTCAAGACACCGAGTCTTTCTCCCTTAAGGCTAGCCCGGTCCCACGAACGTGGAACTGGCAACTAGTGGTGT
CACTACACGCCTCCGACCTCGGACGCGGAGTGCTGTTCCCCAAGCTGTAACCCTGACCCAAGACTGTGCTGCCTGG
CAAGCACCGTCTGGGAAGATGTTCCGCTGTGGCTGCCAAACCTGGTAACAGGTGCCCCAGTGTGTGTAGTCTTCCT
CCAGTCTCCGGACTGGCAGTCTTGTGTAAAGATGCAGTGTAAGGTTCAAGTGCCAAATCCCTGGAAGGAGTGACCC
TCTACTGCCCTAGGAATGCTGTGCAGGTACCCCCAACTTCGGTTGGGGATCTGAGCACAGGCTAATTGTCTACGGG
TAGTTTCATTTCCCATCCTCTCTTTTTTGGCATCATGGCGAACATGGCTCTGCCTGTGACAGCTCTGCTGCTGCCT
CTGGCTCTGCTTCTGCATGCCGCCAGACCTGACATCCAGATGACCCAGACAACCAGCAGCCTGTCTGCCAGCCTGG
GCGACAGAGTGACCATCAGCTGTAGAGCCAGCCAGGACATCAGCAAGTACCTGAACTGGTATCAGCAGAAACCCGA
CGGCACCGTGAAGCTGCTGATCTACCACACCAGCAGACTGCACAGCGGCGTGCCATCTAGATTTTCTGGCAGCGGC
TCTGGCACCGACTACAGCCTGACAATCAGCAACCTGGAACAAGAGGATATCGCTACCTACTTCTGCCAGCAAGGCA
ACACCCTGCCTTACACATTCGGCGGAGGCACCAAGCTGGAAATCACAGGTGGCGGAGGATCTGGCGGAGGTGGAAG
CGGAGGCGGTGGATCTGAAGTGAAACTGCAAGAGTCTGGCCCTGGCCTGGTGGCTCCATCTCAGTCTCTGAGCGTG
ACCTGTACCGTCAGCGGAGTGTCTCTGCCTGATTACGGCGTGTCCTGGATCAGACAGCCTCCTAGAAAAGGCCTGG
AATGGCTGGGCGTGATCTGGGGCAGCGAGACAACCTACTACAACAGCGCCCTGAAGTCCAGGCTGACCATCATCAA
GGACAACTCCAAGAGCCAGGTGTTCCTGAAGATGAACAGCCTGCAGACCGACGACACCGCCATCTACTACTGCGCC
AAGCACTACTACTACGGCGGCAGCTACGCCATGGATTACTGGGGCCAGGGAACCAGCGTGACCGTGTCTAGCACAA
CAACCCCTGCTCCTAGACCTCCTACACCAGCTCCTACAATCGCCAGCCAGCCTCTGTCTCTGAGGCCAGAGGCTTG
TAGACCTGCTGCTGGCGGAGCTGTGCACACAAGAGGCCTGGATTTCGCCTGCGACATCTACATCTGGGCCCCTCTG
GCTGGAACATGTGGCGTGCTGCTGCTGAGCCTGGTCATCACCCTGTATTGCAAGAGAGGCCGGAAGAAGCTGCTCT
ACATCTTCAAGCAGCCCTTCATGAGGCCCGTGCAGACCACACAAGAAGAGGACGGCTGCTCCTGCAGATTCCCCGA
GGAAGAGGAAGGCGGCTGCGAGCTGAGAGTGAAGTTCAGCAGAAGCGCTGACGCCCCTGCTTACCAGCAGGGACAG
AACCAGCTGTACAACGAGCTGAACCTGGGGAGAAGAGAAGAGTACGACGTGCTGGACAAGCGGAGAGGCAGAGATC
CTGAGATGGGCGGCAAGCCCAGAAGAAAGAACCCTCAAGAGGGCCTGTATAATGAGCTGCAGAAAGACAAGATGGC
CGAGGCCTACAGCGAGATCGGAATGAAGGGCGAGCGCAGAAGAGGCAAGGGACACGATGGACTGTACCAGGGCCTG
AGCACAGCCACCAAGGACACATATGACGCCCTGCACATGCAGGCCCTGCCTCCAAGATAATAGAAAAACAAAAAAC
AAAAACCACACAAATGGTCGCCGAAACTACTGAAAGCAT (SEQ ID NO: 226)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCCCCCCTCACCCTCTTTTCCGGCGGCGCATGTTCGCGTCGTCGTA
AGTCTGGATTCCCAAGGCCCACTCGGTTCAACTTCGGTTTCCGGACAAATACAAAGAACCTCAGTCCTCTTGGTAC
TTTCTCGCCTGAGTCTACAAAGCGAGAAACCTGCCCCTCTAACGCCAGACGAGCGGCATAAAACTCGAACTTCTGG
CACGTTCCACCACCCCTTCTCCTATCCCAACCCCCATTGCGCTCTCAAGGTCGCGCTTTTCCGAGACTAGCTCGGA
TTCAAAAAGTTCCTGGCACCCTTTACCCCTTCAGGCCCTTAAGGTAGGAACTGACCTTGTGCTGTGATCTCGGTGC
GGAAGTGCTACTGCGTAGTGATTGTAAGATCCTTTTGTGGTTCTGCCCTGGCAAGGCTACAGAGTGCTGTGATCCG
CTGCGGATGCCATCCTGGTAACAGGACCCCCAGTGTGCGCAACAGTATGTTCACGGTCTTCCGTGTTCACCACATT
CGGAACACTGCTTTCGTGAAACAGTGTGTGTCCAATCCCTGTGATCAGTATCAACCACACCACCTAGGAATGCTAG
GAAGGTACCCCGGTTCGCCGGGATCTGATCCTAGGCTAATTGTCTACGGTGGTGCTCCTTTTTATTTTCCACTTCA
ACTCACTGGTTACAACTGCTTGATTTCTGTGTTTGCTGCTTTTCTCTGCTCTCACTGCCATTCTCAAGTGTTCACA
CTGTCCAAGCTCCTTTGGTTGTTCGCTTCCACTTGCCACTGTCAACTCTTGTCATGGCTCTGCCTGTGACAGCTCT
GCTGCTGCCTCTGGCTCTGCTTCTGCATGCCGCCAGACCTGACATCCAGATGACCCAGACAACCAGCAGCCTGTCT
GCCAGCCTGGGCGACAGAGTGACCATCAGCTGTAGAGCCAGCCAGGACATCAGCAAGTACCTGAACTGGTATCAGC
AGAAACCCGACGGCACCGTGAAGCTGCTGATCTACCACACCAGCAGACTGCACAGCGGCGTGCCATCTAGATTTTC
TGGCAGCGGCTCTGGCACCGACTACAGCCTGACAATCAGCAACCTGGAACAAGAGGATATCGCTACCTACTTCTGC
CAGCAAGGCAACACCCTGCCTTACACATTCGGCGGAGGCACCAAGCTGGAAATCACAGGTGGCGGAGGATCTGGCG
GAGGTGGAAGCGGAGGCGGTGGATCTGAAGTGAAACTGCAAGAGTCTGGCCCTGGCCTGGTGGCTCCATCTCAGTC
TCTGAGCGTGACCTGTACCGTCAGCGGAGTGTCTCTGCCTGATTACGGCGTGTCCTGGATCAGACAGCCTCCTAGA
AAAGGCCTGGAATGGCTGGGCGTGATCTGGGGCAGCGAGACAACCTACTACAACAGCGCCCTGAAGTCCAGGCTGA
CCATCATCAAGGACAACTCCAAGAGCCAGGTGTTCCTGAAGATGAACAGCCTGCAGACCGACGACACCGCCATCTA
CTACTGCGCCAAGCACTACTACTACGGCGGCAGCTACGCCATGGATTACTGGGGCCAGGGAACCAGCGTGACCGTG
TCTAGCACAACAACCCCTGCTCCTAGACCTCCTACACCAGCTCCTACAATCGCCAGCCAGCCTCTGTCTCTGAGGC
CAGAGGCTTGTAGACCTGCTGCTGGCGGAGCTGTGCACACAAGAGGCCTGGATTTCGCCTGCGACATCTACATCTG
GGCCCCTCTGGCTGGAACATGTGGCGTGCTGCTGCTGAGCCTGGTCATCACCCTGTATTGCAAGAGAGGCCGGAAG
AAGCTGCTCTACATCTTCAAGCAGCCCTTCATGAGGCCCGTGCAGACCACACAAGAAGAGGACGGCTGCTCCTGCA
GATTCCCCGAGGAAGAGGAAGGCGGCTGCGAGCTGAGAGTGAAGTTCAGCAGAAGCGCTGACGCCCCTGCTTACCA
GCAGGGACAGAACCAGCTGTACAACGAGCTGAACCTGGGGAGAAGAGAAGAGTACGACGTGCTGGACAAGCGGAGA
GGCAGAGATCCTGAGATGGGCGGCAAGCCCAGAAGAAAGAACCCTCAAGAGGGCCTGTATAATGAGCTGCAGAAAG
ACAAGATGGCCGAGGCCTACAGCGAGATCGGAATGAAGGGCGAGCGCAGAAGAGGCAAGGGACACGATGGACTGTA
CCAGGGCCTGAGCACAGCCACCAAGGACACATATGACGCCCTGCACATGCAGGCCCTGCCTCCAAGATAATAGAAA
AACAAAAAACAAAAACCACACAAATGGTCGCCGAAACTACTGAAAGCAT (SEQ ID NO: 227)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCTAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAAGAGAGGCCGGAAGAAGCTGCTCTACATCTTCAAGCAGCCCTTC
ATGAGGCCCGTGCAGACCACACAAGAAGAGGACGGCTGCTCCTGCAGATTCCCCGAGGAAGAGGAAGGCGGCTGCG
AGCTGAGAGTGAAGTTCAGCAGAAGCGCTGACGCCCCTGCTTACCAGCAGGGACAGAACCAGCTGTACAACGAGCT
GAACCTGGGGAGAAGAGAAGAGTACGACGTGCTGGACAAGCGGAGAGGCAGAGATCCTGAGATGGGCGGCAAGCCC
AGAAGAAAGAACCCTCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTACCAGGGCCTGAGCACAGCCACCAAGGACAC
ATATGACGCCCTGCACATGCAGGCCCTGCCTCCAAGATAATAGAAAAACACAAACCAAACACCATTGTCACACTCC
AAAAAAAACAAACAACCACACAAATGGTCGCCGAAACTACTGAAAGCAT (SEQ ID NO: 228)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCTAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAAGAGAGGCCGGAAGAAGCTGCTCTACATCTTCAAGCAGCCCTTC
ATGAGGCCCGTGCAGACCACACAAGAAGAGGACGGCTGCTCCTGCAGATTCCCCGAGGAAGAGGAAGGCGGCTGCG
AGCTGAGAGTGAAGTTCAGCAGAAGCGCTGACGCCCCTGCTTACCAGCAGGGACAGAACCAGCTGTACAACGAGCT
GAACCTGGGGAGAAGAGAAGAGTACGACGTGCTGGACAAGCGGAGAGGCAGAGATCCTGAGATGGGCGGCAAGCCC
AGAAGAAAGAACCCTCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTACCAGGGCCTGAGCACAGCCACCAAGGACAC
ATATGACGCCCTGCACATGCAGGCCCTGCCTCCAAGATAATAGAAAAACACAAACCAAACACCATTGTCACACTCC
AACTCATGATCAGTACAGAACAAACACCATTGTCACACTCCAAGCTGACATACATATCATAACTCTACTATAGACA
AACACCATTGTCACACTCCAAAAAAAACAAACAACCACACAAATGGTCGCCGAAACTACTGAAAGCAT (SEQ ID
NO: 229)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGCTGCTGCTGGTGACAAGCCTGCTGCTGTGCGAGCTGCCCCACCCCGCCTT
TCTGCTGATCCCCGACATCCAGATGACCCAGACCACCAGCAGCCTGAGCGCCAGCCTGGGCGACAGAGTGACCATC
AGCTGCCGGGCCAGCCAGGACATCAGCAAGTACCTGAACTGGTATCAGCAGAAACCCGACGGCACCGTGAAGCTGC
TGATCTACCACACCTCCCGGCTGCACAGCGGCGTGCCCAGCAGATTTTCTGGCAGCGGCAGCGGCACCGACTACAG
CCTGACCATCTCCAACCTGGAACAAGAAGATATCGCTACCTATTTTTGTCAACAAGGCAACACCCTGCCCTACACC
TTCGGCGGAGGCACCAAACTGGAAATCACCGGCAGCACCAGCGGCTCCGGCAAGCCTGGATCTGGCGAGGGCAGCA
CCAAGGGCGAAGTGAAGCTGCAAGAAAGCGGCCCTGGCCTGGTGGCCCCTAGCCAGAGCCTGTCCGTGACCTGTAC
CGTGTCCGGCGTGTCCCTGCCCGACTACGGCGTGTCCTGGATCCGGCAGCCCCCCAGAAAGGGCCTGGAATGGCTG
GGCGTGATCTGGGGCAGCGAGACAACCTACTACAACAGCGCCCTGAAGTCCCGGCTGACCATCATCAAGGACAACA
GCAAGAGCCAGGTGTTCCTGAAGATGAACAGCCTGCAGACCGACGACACCGCCATCTACTACTGCGCCAAGCACTA
CTACTACGGCGGCAGCTACGCCATGGACTACTGGGGCCAGGGCACCAGCGTGACCGTGTCCAGCTTCGTGCCCGTG
TTTCTGCCCGCCAAGCCTACCACCACCCCTGCCCCTAGACCTCCCACCCCAGCCCCAACAATCGCCAGCCAGCCTC
TGTCCCTGCGGCCCGAAGCCTGTAGACCAGCTGCCGGCGGAGCCGTGCACACCAGAGGCCTGGACTTCGCCTGCGA
CATCTACATCTGGGCCCCTCTGGCCGGCACCTGTGGCGTGCTGCTGCTGAGCCTGGTGATCACCCTGTACTGCAAC
CACCGGAACAGAAGCAAGCGGAGCCGGCTGCTGCACAGCGACTACATGAACATGACCCCAAGACGGCCTGGCCCCA
CCCGGAAGCACTACCAGCCTTACGCCCCTCCCAGAGACTTCGCCGCCTACCGGTCCAGAGTGAAGTTCAGCAGATC
CGCCGACGCCCCTGCCTACCAGCAGGGACAGAACCAGCTGTACAACGAGCTGAACCTGGGCAGACGGGAAGAGTAC
GACGTGCTGGACAAGCGGAGAGGCCGGGACCCCGAGATGGGCGGAAAGCCCAGACGGAAGAACCCCCAGGAAGGCC
TGTATAACGAACTGCAGAAAGACAAGATGGCCGAGGCCTACAGCGAGATCGGCATGAAGGGCGAGCGGAGGCGCGG
CAAGGGCCACGATGGCCTGTACCAGGGCCTGAGCACCGCCACCAAGGACACCTACGACGCCCTGCACATGCAGGCC
CTGCCCCCCAGATGATAATAGAAAAACAAAAAACAAAAACCACACAAATGGTCGCCGAAACTACTGAAAGCAT
(SEQ ID NO: 230)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCATTACCAGTTACAGCCCTCCTCCTCCCTCTCGCTCTCCTCCTCCACGC
CGCCAGGCCAGATATCCAGATGACACAGACCACCAGCAGCCTCAGCGCCAGCCTCGGCGATAGAGTGACAATCTCT
TGCAGAGCCTCCCAGGACATCAGCAAGTACCTCAATTGGTACCAGCAGAAGCCCGACGGCACCGTGAAGCTCCTGA
TCTACCACACCAGCAGACTGCACAGCGGCGTGCCTTCTAGATTCTCTGGAAGCGGCAGCGGCACCGACTACAGCCT
GACCATCAGCAACCTGGAGCAGGAGGACATCGCCACCTACTTCTGCCAGCAGGGCAATACCCTGCCCTACACCTTC
GGCGGCGGCACAAAACTGGAAATTACAGGTGGTGGTGGTTCTGGGGGAGGAGGGTCTGGTGGTGGTGGTAGTGAAG
TGAAACTGCAGGAATCTGGACCAGGACTGGTGGCCCCATCTCAGTCTCTGAGCGTGACATGTACAGTGTCTGGCGT
GTCTCTGCCTGATTACGGAGTGTCTTGGATTAGACAGCCTCCTCGGAAGGGCCTGGAGTGGCTGGGAGTGATCTGG
GGCAGCGAGACCACCTACTACAACAGCGCCCTGAAGTCCAGGCTGACCATCATCAAGGACAACAGCAAGTCCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGCACCAGCGTGACGGTGTCTTCTACAACAACACCTGCTCCTAGGCCT
CCTACACCAGCTCCTACAATTGCTTCTCAGCCGCTGTCTCTGCGGCCAGAAGCTTGTAGGCCTGCTGCAGGAGGAG
CGGTGCATACAAGAGGACTGGACTTTGCCTGTGATATCTACATCTGGGCCCCTCTGGCCGGCACCTGCGGCGTGCT
GCTGCTGAGCCTGGTGATCACCCTGTACTGCAACCACAGAAACAGAAGCAAGAGAAGCAGGCTGCTGCACAGCGAC
TACATGAATATGACACCTAGGAGACCTGGCCCTACAAGAAAACATTATCAGCCTTATGCCCCTCCTAGAGATTTTG
CCGCCTATAGAAGCCGGGTGAAGTTTTCTAGATCTGCTGATGCCCCAGCCTATCAGCAGGGCCAGAACCAGTTATA
TAATGAACTGAACCTGGGCCGGCGGGAGGAGTACGATGTGCTGGATAAGAGAAGAGGCAGAGATCCTGAAATGGGA
GGAAAACCTAGAAGAAAGAATCCCCAGGAGGGCCTATATAACGAACTGCAGAAGGACAAGATGGCCGAGGCCTACA
GCGAGATCGGCATGAAGGGCGAGAGAAGAAGAGGCAAGGGCCACGATGGACTGTATCAGGGGCTGAGCACCGCCAC
CAAGGATACCTACGATGCCCTGCACATGCAGGCCCTGCCTCCTAGATAATAGAAAAACAAAAAACAAAAACCACAC
AAATGGTCGCCGAAACTACTGAAAGCAT (SEQ ID NO: 231)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGCTATTATTAGTCACCAGCCTCCTCCTTTGCGAGCTCCCCCACCCCGCCTT
CCTCCTCATCCCCGACATCCAGATGACCCAGACCACCAGCAGCCTCAGCGCCAGCCTCGGCGATAGAGTGACAATC
TCTTGCAGAGCCTCCCAGGACATCAGCAAGTACCTCAATTGGTACCAGCAGAAGCCCGACGGCACCGTGAAGCTGC
TGATCTACCACACCAGCAGACTGCACAGCGGCGTGCCTTCTAGATTCTCTGGAAGCGGCAGCGGCACCGACTACAG
CCTGACCATCAGCAACCTGGAGCAGGAGGACATCGCCACCTACTTCTGCCAGCAGGGCAATACCTTGCCCTACACC
TTCGGCGGCGGCACAAAGCTGGAAATTACAGGTGGTGGTGGTAGTGGTGGTGGTGGTTCTGGTGGTGGTGGATCTG
AAGTGAAACTGCAGGAATCTGGACCTGGCCTGGTGGCCCCTTCTCAGTCGCTGAGCGTGACATGTACAGTGAGCGG
CGTGAGCCTGCCTGATTATGGAGTGAGCTGGATCAGACAGCCTCCAAGAAAGGGCCTGGAGTGGCTGGGCGTGATC
TGGGGCTCCGAGACCACCTACTACAACAGCGCCCTGAAGTCCAGGCTGACCATCATCAAGGACAACAGCAAGAGCC
AGGTGTTCCTGAAGATGAACAGCCTGCAGACCGATGACACCGCCATCTACTACTGCGCCAAGCACTACTACTACGG
CGGCAGCTACGCCATGGATTACTGGGGCCAGGGCACATCTGTGACAGTGTCTTCTGCTGCTGCTCCAGCTCCTAGA
CCTCCTACACCTGCTCCTACAATTGCTTCTCAGCCGCTGAGCCTGAGACCTGAAGCTTGTAGACCTGCTGCTGGAG
GAGCTGTGCATACAAGAGGACTGGATTTTGATACCGCCCTGGCCGCCGTGATCTGTTCTGCCCTGGCCACAGTGCT
GCTGGCCCTGCTGATCCTGTGCGTGATCTACTGCAAGAGACAGCCCAGAAGAAAGAAGCTGCTGTACATCTTCAAG
CAGCCCTTCATGAGACCTGTGCAGACAACACAGGAGGAGGATGGCTGTTCCTGTAGATTTCCAGAGGAAGAGGAGG
GCGGCTGCGAGCTGAGAGTGAAATTTTCTAGATCTGCCGATGCCCCTGCCTACCAGCAGGGCCAGAACCAGCTATA
TAATGAACTGAACCTGGGCAGAAGAGAGGAGTACGACGTGCTGGATAAGAGAAGAGGCAGAGATCCTGAAATGGGA
GGAAAACCTAGAAGAAAGAATCCGCAGGAGGGCCTATATAACGAACTGCAGAAGGACAAGATGGCCGAGGCCTACA
GCGAGATCGGCATGAAGGGCGAGAGAAGAAGAGGCAAGGGCCACGATGGACTGTATCAGGGGCTGAGCACAGCCAC
CAAGGATACCTACGATGCCCTGCACATGCAGGCCCTGCCTCCTAGATAATAGAAAAACAAAAAACAAAAACCACAC
AAATGGTCGCCGAAACTACTGAAAGCAT (SEQ ID NO: 232)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTACCCGTTACAGCATTACTACTACCTCTTGCCCTCCTCCTCCACGC
CGCCAGACCAGATATCCAGATGACCCAGACCACCTCTAGCCTCAGCGCCAGCCTCGGCGATAGAGTGACAATCTCT
TGCAGAGCCTCCCAGGACATCAGCAAGTACCTCAATTGGTACCAGCAGAAGCCCGACGGCACCGTGAAGCTCCTGA
TCTACCACACCAGCAGGCTGCACTCTGGCGTGCCATCTAGATTTTCTGGAAGCGGCAGCGGCACAGATTACAGCCT
GACCATCAGCAATCTGGAGCAGGAGGACATCGCCACCTACTTCTGCCAGCAGGGCAATACCCTGCCCTACACCTTC
GGCGGCGGCACAAAACTGGAAATTACAGGTGGTGGTGGTTCAGGGGGAGGAGGGTCTGGTGGTGGAGGTTCTGAAG
TGAAACTGCAGGAATCTGGACCTGGCCTGGTGGCCCCTTCTCAGTCGCTGAGCGTGACATGTACCGTGAGCGGCGT
GAGCCTGCCTGATTACGGGGTGAGCTGGATCAGACAGCCCCCTAGAAAGGGCCTGGAATGGCTGGGCGTGATCTGG
GGCAGCGAGACCACCTACTACAACAGCGCCCTGAAGTCCAGGCTGACCATCATCAAGGACAACAGCAAGTCCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGCACCTCTGTGACAGTGAGCAGCGCCGCTGCCATTGAGGTGATGTAC
CCTCCCCCCTACCTGGACAACGAGAAGTCCAACGGCACCATCATCCACGTGAAGGGCAAGCACCTGTGCCCTAGCC
CCCTGTTTCCAGGCCCATCTAAGCCTTTTTGGGTGCTGGTGGTGGTGGGCGGCGTGCTGGCCTGCTACAGCCTGCT
GGTGACCGTGGCCTTCATCATCTTCTGGGTGAGAAGCAAGAGGTCCCGGCTGCTGCACAGCGACTACATGAATATG
ACACCCAGGAGACCTGGCCCTACAAGAAAACATTATCAGCCTTATGCCCCTCCTAGAGATTTTGCCGCCTATAGAA
GCCGGGTGAAGTTTTCTAGAAGCGCTGATGCCCCAGCCTACCAGCAGGGCCAGAACCAGCTGTACAACGAGCTGAA
CCTGGGCAGAAGAGAGGAGTACGACGTGCTGGATAAGAGAAGAGGCAGAGATCCGGAAATGGGAGGAAAGCCTAGA
AGAAAGAATCCTCAGGAGGGCCTGTACAATGAGCTGCAGAAGGACAAGATGGCCGAGGCCTACTCTGAGATCGGCA
TGAAGGGCGAGAGAAGAAGAGGCAAGGGCCACGATGGCCTGTACCAGGGCCTGAGCACGGCCACCAAGGATACCTA
CGATGCCCTGCACATGCAGGCCCTGCCTCCTAGATAATAGAAAAACAAAAAACAAAAACCACACAAATGGTCGCCG
AAACTACTGAAAGCAT (SEQ ID NO: 233)
*Because the sequences are RNA sequences, it is understood that T's represent uracil in the sequences of this table.

In some aspects, the disclosure provides circRNA constructs comprising a codon-optimized sequence comprising an anti-CD19 antibody or antigen binding domain. Table 7A describes for each full-length circRNA sequence (as described in Table 6), the corresponding codon-optimized sequence (comprising the anti-CD19 antibody or antigen binding domain) within the full-length circRNA sequence, and the sequence of the encoded anti-CD19 protein (as described in Table 5). For example, in the first row of Table 7A, SEQ ID NO: 175 is an exemplary full-length circRNA, that comprises a codon-optimized RNA sequence of SEQ ID NO: 125, encoding an anti-CD19 antibody or antigen binding domain that is translated into the anti-CD19 protein of SEQ ID NO: 169. In some embodiments, the coding sequence encoding a CAR comprises SEQ ID NO: 125.

TABLE 7A
Full-length circRNAs sequences (Table 6) and corresponding
exemplary codon-optimized coding sequence constructs (Table 4), and the
encoded anti-CD19 antibody or antigen-binding domain sequence (Table 5)

| | Codon-optimized coding | Encoded |
|---|---|---|
| Full-length | sequence comprising Anti- | Anti-CD19 |
| circRNA | CD19 antibody or antigen | Protein |
| (Table 6) | binding domain (Table 4) | (Table 5) |
| SEQ ID NO: 175 | SEQ ID NO: 125 | SEQ ID NO: 169 |
| SEQ ID NO: 214 | SEQ ID NO: 125 | SEQ ID NO: 169 |
| SEQ ID NO: 215 | SEQ ID NO: 125 | SEQ ID NO: 169 |
| SEQ ID NO: 217 | SEQ ID NO: 125 | SEQ ID NO: 169 |
| SEQ ID NO: 218 | SEQ ID NO: 125 | SEQ ID NO: 169 |
| SEQ ID NO: 219 | SEQ ID NO: 125 | SEQ ID NO: 169 |
| SEQ ID NO: 220 | SEQ ID NO: 125 | SEQ ID NO: 169 |
| SEQ ID NO: 221 | SEQ ID NO: 125 | SEQ ID NO: 169 |
| SEQ ID NO: 222 | SEQ ID NO: 125 | SEQ ID NO: 169 |
| SEQ ID NO: 223 | SEQ ID NO: 125 | SEQ ID NO: 169 |
| SEQ ID NO: 224 | SEQ ID NO: 125 | SEQ ID NO: 169 |
| SEQ ID NO: 225 | SEQ ID NO: 125 | SEQ ID NO: 169 |
| SEQ ID NO: 226 | SEQ ID NO: 125 | SEQ ID NO: 169 |
| SEQ ID NO: 227 | SEQ ID NO: 125 | SEQ ID NO: 169 |
| SEQ ID NO: 228 | SEQ ID NO: 125 | SEQ ID NO: 169 |
| SEQ ID NO: 229 | SEQ ID NO: 125 | SEQ ID NO: 169 |
| SEQ ID NO: 176 | SEQ ID NO: 126 | SEQ ID NO: 169 |
| SEQ ID NO: 177 | SEQ ID NO: 127 | SEQ ID NO: 169 |
| SEQ ID NO: 178 | SEQ ID NO: 128 | SEQ ID NO: 169 |
| SEQ ID NO: 179 | SEQ ID NO: 129 | SEQ ID NO: 169 |
| SEQ ID NO: 180 | SEQ ID NO: 130 | SEQ ID NO: 169 |
| SEQ ID NO: 181 | SEQ ID NO: 131 | SEQ ID NO: 169 |
| SEQ ID NO: 182 | SEQ ID NO: 132 | SEQ ID NO: 169 |
| SEQ ID NO: 183 | SEQ ID NO: 133 | SEQ ID NO: 169 |
| SEQ ID NO: 184 | SEQ ID NO: 134 | SEQ ID NO: 169 |
| SEQ ID NO: 185 | SEQ ID NO: 135 | SEQ ID NO: 169 |
| SEQ ID NO: 186 | SEQ ID NO: 136 | SEQ ID NO: 169 |
| SEQ ID NO: 187 | SEQ ID NO: 137 | SEQ ID NO: 169 |
| SEQ ID NO: 188 | SEQ ID NO: 138 | SEQ ID NO: 169 |
| SEQ ID NO: 189 | SEQ ID NO: 139 | SEQ ID NO: 169 |
| SEQ ID NO: 190 | SEQ ID NO: 140 | SEQ ID NO: 169 |
| SEQ ID NO: 191 | SEQ ID NO: 141 | SEQ ID NO: 169 |
| SEQ ID NO: 192 | SEQ ID NO: 142 | SEQ ID NO: 169 |
| SEQ ID NO: 193 | SEQ ID NO: 143 | SEQ ID NO: 169 |
| SEQ ID NO: 194 | SEQ ID NO: 144 | SEQ ID NO: 169 |
| SEQ ID NO: 195 | SEQ ID NO: 145 | SEQ ID NO: 169 |
| SEQ ID NO: 196 | SEQ ID NO: 146 | SEQ ID NO: 169 |
| SEQ ID NO: 197 | SEQ ID NO: 147 | SEQ ID NO: 169 |
| SEQ ID NO: 198 | SEQ ID NO: 148 | SEQ ID NO: 169 |
| SEQ ID NO: 199 | SEQ ID NO: 149 | SEQ ID NO: 169 |
| SEQ ID NO: 200 | SEQ ID NO: 150 | SEQ ID NO: 169 |
| SEQ ID NO: 201 | SEQ ID NO: 151 | SEQ ID NO: 169 |
| SEQ ID NO: 202 | SEQ ID NO: 152 | SEQ ID NO: 169 |
| SEQ ID NO: 203 | SEQ ID NO: 153 | SEQ ID NO: 169 |
| SEQ ID NO: 204 | SEQ ID NO: 154 | SEQ ID NO: 169 |
| SEQ ID NO: 205 | SEQ ID NO: 155 | SEQ ID NO: 169 |
| SEQ ID NO: 206 | SEQ ID NO: 156 | SEQ ID NO: 169 |
| SEQ ID NO: 207 | SEQ ID NO: 157 | SEQ ID NO: 169 |
| SEQ ID NO: 208 | SEQ ID NO: 158 | SEQ ID NO: 169 |
| SEQ ID NO: 209 | SEQ ID NO: 159 | SEQ ID NO: 169 |
| SEQ ID NO: 210 | SEQ ID NO: 160 | SEQ ID NO: 169 |
| SEQ ID NO: 211 | SEQ ID NO: 161 | SEQ ID NO: 169 |
| SEQ ID NO: 212 | SEQ ID NO: 162 | SEQ ID NO: 169 |
| SEQ ID NO: 213 | SEQ ID NO: 163 | SEQ ID NO: 169 |
| SEQ ID NO: 216 | SEQ ID NO: 164 | SEQ ID NO: 170 |
| SEQ ID NO: 230 | SEQ ID NO: 165 | SEQ ID NO: 171 |
| SEQ ID NO: 231 | SEQ ID NO: 166 | SEQ ID NO: 172 |
| SEQ ID NO: 232 | SEQ ID NO: 167 | SEQ ID NO: 173 |
| SEQ ID NO: 233 | SEQ ID NO: 168 | SEQ ID NO: 174 |

In some aspects, the disclosure provides exemplary circRNA constructs with a combination of elements. Table 7B provides two exemplary circRNA sequences with an annotation of the specific combination of precisely engineered elements (5′ UTR, IRES, codon-optimized coding sequence encoding a protein sequence, and 3′ UTR (including microRNA binding sites)). Table 7B also provides the amino acid sequence of the anti-CD19 antibody or antigen-binding fragment that is encoded by the codon-optimized coding sequence of the circRNA.

In some embodiments, the circRNA comprises a sequence according to SEQ ID NO: 264.

In some aspects, provided herein is a circRNA comprising a sequence having at least 85% identity to SEQ ID NO: 264. In some embodiments, provided herein is a circRNA comprising a sequence having at least 90% identity to SEQ ID NO: 264. In some embodiments, provided herein is a circRNA comprising a sequence having at least 95% identity to SEQ ID NO: 264. In some embodiments, provided herein is a circRNA comprising a sequence having at least 98% identity to SEQ ID NO: 264. In some embodiments, provided herein is a circRNA comprising a sequence having at least 99% identity to SEQ ID NO: 264. In some embodiments, the circRNA comprises a sequence of SEQ ID NO: 264.

In some embodiments, provided herein is a circRNA therapeutic comprising a circRNA sequence according to SEQ ID NO: 264.

In some embodiments, the circRNA comprises a sequence according to SEQ ID NO: 269. In some embodiments, provided herein is a circRNA therapeutic comprising a circRNA sequence according to SEQ ID NO: 269.

TABLE 7B
Exemplary circRNA Sequences*, and sequence for the encoded protein
within the circRNA

AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCCAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAAGAGAGGCCGGAAGAAGCTGCTCTACATCTTCAAGCAGCCCTTC
ATGAGGCCCGTGCAGACCACACAAGAAGAGGACGGCTGCTCCTGCAGATTCCCCGAGGAAGAGGAAGGCGGCTGCG
AGCTGAGAGTGAAGTTCAGCAGAAGCGCTGACGCCCCTGCTTACCAGCAGGGACAGAACCAGCTGTACAACGAGCT
GAACCTGGGGAGAAGAGAAGAGTACGACGTGCTGGACAAGCGGAGAGGCAGAGATCCTGAGATGGGCGGCAAGCCC
AGAAGAAAGAACCCTCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTACCAGGGCCTGAGCACAGCCACCAAGGACAC
ATATGACGCCCTGCACATGCAGGCCCTGCCTCCAAGATAATAGaacaaacacaaacCAAACACCATTGTCACACTC
CAaacaacaaacaaacaACCACACAAATGGTCGCCGAaactactgaaagcat (SEQ ID NO: 264)
5' UTR (Italics) with 5' exon fragment (Italics and underlined):
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATC (SEQ ID NO: 265)
5' exon fragment sequence is in italics and underlined.
5' UTR (Italics):
TCGGCGACCATTTGTGTGGTGAGCTCTAAGTGTTGATCAGCGGGCGCAGCTGCATATGACCTCTGCGTGTGCCTGG
TAATTGACTAAGATATC (SEQ ID NO: 348)
IRES263 (Bold Italics):
TTACTCAGCGTAACTACTCCGGGTTACGTGATGAAGAAGAGGCTACGGAGATTCTCGGGCTACGGCCCTGGAGCCA
CTCCGGCTCCTAAAGATTTAGAAGTTTGAGCACACCCGCCCACTAGGGCCCCCCATCCAGGGGGGCAACGGGCAAG
CACTTCTGTTTCCCCGGTATGATCTGATAGGCTGTAACCACGGCTGAAACAGAGATTATCGTTATCCGCTTCACTA
CTTCGAGAAGCCTAGTAATGATGGGTGAAATTGAATCCGTTGATCCGGTGTCTCCCCCACACCAGAAACTCATGAT
GAGGGTTGCCATCCCGGCTACGGCGACGTAGCGGGCATCCCTGCGCTGGCATGAGGCCTCTTAGGAGGACGGATGA
TATGGATCTTGTCGTGAAGAGCCTATTGAGCTAGTGTCGACTCCTCCGCCCCCGTGAATGCGGCTAATCCTAACCC
CGGAGCAGGTGGGTCCAATCCAGGGCCTGGCCTGTCGTAATGCGTAAGTCTGGGACGGAACCGACTACTTTCGGGA
AGGCGTGTTTCCATTTGTTCATTATTTGTGTGTTTATGGTGACAACTCTGGGTAAACGTTCTATTGCGTTTATTGA
GAGATTCCCAACAATTGAACAAACGAGAACTACCTGTTTTATTAAATTTACACAGAGAAGAATTACA (SEQ ID
NO: 1)
Codon-optimized Coding Sequence:
ATGGCTCTGCCTGTGACAGCTCTGCTGCTGCCTCTGGCTCTGCTTCTGCATGCCGCCAGACCTGACATCCAGATGA
CCCAGACAACCAGCAGCCTGTCTGCCAGCCTGGGCGACAGAGTGACCATCAGCTGTAGAGCCAGCCAGGACATCAG
CAAGTACCTGAACTGGTATCAGCAGAAACCCGACGGCACCGTGAAGCTGCTGATCTACCACACCAGCAGACTGCAC
AGCGGCGTGCCATCCAGATTTTCTGGCAGCGGCTCTGGCACCGACTACAGCCTGACAATCAGCAACCTGGAACAAG
AGGATATCGCTACCTACTTCTGCCAGCAAGGCAACACCCTGCCTTACACATTCGGCGGAGGCACCAAGCTGGAAAT
CACAGGTGGCGGAGGATCTGGCGGAGGTGGAAGCGGAGGCGGTGGATCTGAAGTGAAACTGCAAGAGTCTGGCCCT
GGCCTGGTGGCTCCATCTCAGTCTCTGAGCGTGACCTGTACCGTCAGCGGAGTGTCTCTGCCTGATTACGGCGTGT
CCTGGATCAGACAGCCTCCTAGAAAAGGCCTGGAATGGCTGGGCGTGATCTGGGGCAGCGAGACAACCTACTACAA
CAGCGCCCTGAAGTCCAGGCTGACCATCATCAAGGACAACTCCAAGAGCCAGGTGTTCCTGAAGATGAACAGCCTG
CAGACCGACGACACCGCCATCTACTACTGCGCCAAGCACTACTACTACGGCGGCAGCTACGCCATGGATTACTGGG
GCCAGGGAACCAGCGTGACCGTGTCTAGCACAACAACCCCTGCTCCTAGACCTCCTACACCAGCTCCTACAATCGC
CAGCCAGCCTCTGTCTCTGAGGCCAGAGGCTTGTAGACCTGCTGCTGGCGGAGCTGTGCACACAAGAGGCCTGGAT
TTCGCCTGCGACATCTACATCTGGGCCCCTCTGGCTGGAACATGTGGCGTGCTGCTGCTGAGCCTGGTCATCACCC
TGTATTGCAAGAGAGGCCGGAAGAAGCTGCTCTACATCTTCAAGCAGCCCTTCATGAGGCCCGTGCAGACCACACA
AGAAGAGGACGGCTGCTCCTGCAGATTCCCCGAGGAAGAGGAAGGCGGCTGCGAGCTGAGAGTGAAGTTCAGCAGA
AGCGCTGACGCCCCTGCTTACCAGCAGGGACAGAACCAGCTGTACAACGAGCTGAACCTGGGGAGAAGAGAAGAGT
ACGACGTGCTGGACAAGCGGAGAGGCAGAGATCCTGAGATGGGCGGCAAGCCCAGAAGAAAGAACCCTCAAGAGGG
CCTGTATAATGAGCTGCAGAAAGACAAGATGGCCGAGGCCTACAGCGAGATCGGAATGAAGGGCGAGCGCAGAAGA
GGCAAGGGACACGATGGACTGTACCAGGGCCTGAGCACAGCCACCAAGGACACATATGACGCCCTGCACATGCAGG
CCCTGCCTCCAAGA (SEQ ID NO: 266)
miR-122 binding sequence (Bold underlined): CAAACACCATTGTCACACTCCA (SEQ ID
NO: 40)
miR-122_2 spacer: aacaaacacaaacCAAACACCATTGTCACACTCCAaacaacaaacaaaca (SEQ ID
NO: 267)
3' UTR with 3' exon fragment (lower case and underlined):
TAATAGaacaaacacaaacCAAACACCATTGTCACACTCCAaacaacaaacaaacaACCACACAAATGGTCGCCGAa
actactgaaagcat (SEQ ID NO: 268)
3' exon fragment sequence is in lower case and is underlined.
3' UTR:
TAATAGaacaaacacaaacCAAACACCATTGTCACACTCCAaacaacaaacaaacaACCACACAAATGGTCGCCGA
(SEQ ID NO: 349)
Encoded Anti-CD19 protein:
MALPVTALLLPLALLLHAARPDIQMTOTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDGCSCRFPEEEEGGCELRVKESR
SADAPAYQQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR (SEQ ID NO: 174)
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATCTTACTCAGCGTAACTACTCCGGGTTACGTGATGAAGAAGAGGCT
ACGGAGATTCTCGGGCTACGGCCCTGGAGCCACTCCGGCTCCTAAAGATTTAGAAGTTTGAGCACACCCGCCCACT
AGGGCCCCCCATCCAGGGGGGCAACGGGCAAGCACTTCTGTTTCCCCGGTATGATCTGATAGGCTGTAACCACGGC
TGAAACAGAGATTATCGTTATCCGCTTCACTACTTCGAGAAGCCTAGTAATGATGGGTGAAATTGAATCCGTTGAT
CCGGTGTCTCCCCCACACCAGAAACTCATGATGAGGGTTGCCATCCCGGCTACGGCGACGTAGCGGGCATCCCTGC
GCTGGCATGAGGCCTCTTAGGAGGACGGATGATATGGATCTTGTCGTGAAGAGCCTATTGAGCTAGTGTCGACTCC
TCCGCCCCCGTGAATGCGGCTAATCCTAACCCCGGAGCAGGTGGGTCCAATCCAGGGCCTGGCCTGTCGTAATGCG
TAAGTCTGGGACGGAACCGACTACTTTCGGGAAGGCGTGTTTCCATTTGTTCATTATTTGTGTGTTTATGGTGACA
ACTCTGGGTAAACGTTCTATTGCGTTTATTGAGAGATTCCCAACAATTGAACAAACGAGAACTACCTGTTTTATTA
AATTTACACAGAGAAGAATTACAATGGCTCTGCCTGTGACAGCTCTGCTGCTGCCTCTGGCTCTGCTTCTGCATGC
CGCCAGACCTGACATCCAGATGACCCAGACAACCAGCAGCCTGTCTGCCAGCCTGGGCGACAGAGTGACCATCAGC
TGTAGAGCCAGCCAGGACATCAGCAAGTACCTGAACTGGTATCAGCAGAAACCCGACGGCACCGTGAAGCTGCTGA
TCTACCACACCAGCAGACTGCACAGCGGCGTGCCATCTAGATTTTCTGGCAGCGGCTCTGGCACCGACTACAGCCT
GACAATCAGCAACCTGGAACAAGAGGATATCGCTACCTACTTCTGCCAGCAAGGCAACACCCTGCCTTACACATTC
GGCGGAGGCACCAAGCTGGAAATCACAGGTGGCGGAGGATCTGGCGGAGGTGGAAGCGGAGGCGGTGGATCTGAAG
TGAAACTGCAAGAGTCTGGCCCTGGCCTGGTGGCTCCATCTCAGTCTCTGAGCGTGACCTGTACCGTCAGCGGAGT
GTCTCTGCCTGATTACGGCGTGTCCTGGATCAGACAGCCTCCTAGAAAAGGCCTGGAATGGCTGGGCGTGATCTGG
GGCAGCGAGACAACCTACTACAACAGCGCCCTGAAGTCCAGGCTGACCATCATCAAGGACAACTCCAAGAGCCAGG
TGTTCCTGAAGATGAACAGCCTGCAGACCGACGACACCGCCATCTACTACTGCGCCAAGCACTACTACTACGGCGG
CAGCTACGCCATGGATTACTGGGGCCAGGGAACCAGCGTGACCGTGTCTAGCACAACAACCCCTGCTCCTAGACCT
CCTACACCAGCTCCTACAATCGCCAGCCAGCCTCTGTCTCTGAGGCCAGAGGCTTGTAGACCTGCTGCTGGCGGAG
CTGTGCACACAAGAGGCCTGGATTTCGCCTGCGACATCTACATCTGGGCCCCTCTGGCTGGAACATGTGGCGTGCT
GCTGCTGAGCCTGGTCATCACCCTGTATTGCAAGAGAGGCCGGAAGAAGCTGCTCTACATCTTCAAGCAGCCCTTC
ATGAGGCCCGTGCAGACCACACAAGAAGAGGACGGCTGCTCCTGCAGATTCCCCGAGGAAGAGGAAGGCGGCTGCG
AGCTGAGAGTGAAGTTCAGCAGAAGCGCTGACGCCCCTGCTTACCAGCAGGGACAGAACCAGCTGTACAACGAGCT
GAACCTGGGGAGAAGAGAAGAGTACGACGTGCTGGACAAGCGGAGAGGCAGAGATCCTGAGATGGGCGGCAAGCCC
AGAAGAAAGAACCCTCAAGAGGGCCTGTATAATGAGCTGCAGAAAGACAAGATGGCCGAGGCCTACAGCGAGATCG
GAATGAAGGGCGAGCGCAGAAGAGGCAAGGGACACGATGGACTGTACCAGGGCCTGAGCACAGCCACCAAGGACAC
ATATGACGCCCTGCACATGCAGGCCCTGCCTCCAAGATAATAGaaaaacacaaacCAAACACCATTGTCACACTCC
AaaaaaaacaaacaACCACACAAATGGTCGCCGAaactactgaaagcat
(SEQ ID NO: 269)
5' UTR (Italics) with 5' exon fragment (Italics and underlined):
AACTTTTAGTTATTTTCGGCGACCATTTGTGTGGTGAGCTCTAAGTGTTGATCAGCGGGCGCAGCTGCATATGACC
TCTGCGTGTGCCTGGTAATTGACTAAGATATC (SEQ ID NO: 265)
5' exon fragment sequence is in italics and is underlined
5' UTR (Italics):
TCGGCGACCATTTGTGTGGTGAGCTCTAAGTGTTGATCAGCGGGCGCAGCTGCATATGACCTCTGCGTGTGCCTGG
TAATTGACTAAGATATC (SEQ ID NO: 348)
IRES263 (Bold Italics):
TTACTCAGCGTAACTACTCCGGGTTACGTGATGAAGAAGAGGCTACGGAGATTCTCGGGCTACGGCCCTGGAGCCA
CTCCGGCTCCTAAAGATTTAGAAGTTTGAGCACACCCGCCCACTAGGGCCCCCCATCCAGGGGGGCAACGGGCAAG
CACTTCTGTTTCCCCGGTATGATCTGATAGGCTGTAACCACGGCTGAAACAGAGATTATCGTTATCCGCTTCACTA
CTTCGAGAAGCCTAGTAATGATGGGTGAAATTGAATCCGTTGATCCGGTGTCTCCCCCACACCAGAAACTCATGAT
GAGGGTTGCCATCCCGGCTACGGCGACGTAGCGGGCATCCCTGCGCTGGCATGAGGCCTCTTAGGAGGACGGATGA
TATGGATCTTGTCGTGAAGAGCCTATTGAGCTAGTGTCGACTCCTCCGCCCCCGTGAATGCGGCTAATCCTAACCC
CGGAGCAGGTGGGTCCAATCCAGGGCCTGGCCTGTCGTAATGCGTAAGTCTGGGACGGAACCGACTACTTTCGGGA
AGGCGTGTTTCCATTTGTTCATTATTTGTGTGTTTATGGTGACAACTCTGGGTAAACGTTCTATTGCGTTTATTGA
GAGATTCCCAACAATTGAACAAACGAGAACTACCTGTTTTATTAAATTTACACAGAGAAGAATTACA (SEQ ID
NO: 1)
Codon-optimized Coding Sequence:
ATGGCTCTGCCTGTGACAGCTCTGCTGCTGCCTCTGGCTCTGCTTCTGCATGCCGCCAGACCTGACATCCAGATGA
CCCAGACAACCAGCAGCCTGTCTGCCAGCCTGGGCGACAGAGTGACCATCAGCTGTAGAGCCAGCCAGGACATCAG
CAAGTACCTGAACTGGTATCAGCAGAAACCCGACGGCACCGTGAAGCTGCTGATCTACCACACCAGCAGACTGCAC
AGCGGCGTGCCATCTAGATTTTCTGGCAGCGGCTCTGGCACCGACTACAGCCTGACAATCAGCAACCTGGAACAAG
AGGATATCGCTACCTACTTCTGCCAGCAAGGCAACACCCTGCCTTACACATTCGGCGGAGGCACCAAGCTGGAAAT
CACAGGTGGCGGAGGATCTGGCGGAGGTGGAAGCGGAGGCGGTGGATCTGAAGTGAAACTGCAAGAGTCTGGCCCT
GGCCTGGTGGCTCCATCTCAGTCTCTGAGCGTGACCTGTACCGTCAGCGGAGTGTCTCTGCCTGATTACGGCGTGT
CCTGGATCAGACAGCCTCCTAGAAAAGGCCTGGAATGGCTGGGCGTGATCTGGGGCAGCGAGACAACCTACTACAA
CAGCGCCCTGAAGTCCAGGCTGACCATCATCAAGGACAACTCCAAGAGCCAGGTGTTCCTGAAGATGAACAGCCTG
CAGACCGACGACACCGCCATCTACTACTGCGCCAAGCACTACTACTACGGCGGCAGCTACGCCATGGATTACTGGG
GCCAGGGAACCAGCGTGACCGTGTCTAGCACAACAACCCCTGCTCCTAGACCTCCTACACCAGCTCCTACAATCGC
CAGCCAGCCTCTGTCTCTGAGGCCAGAGGCTTGTAGACCTGCTGCTGGCGGAGCTGTGCACACAAGAGGCCTGGAT
TTCGCCTGCGACATCTACATCTGGGCCCCTCTGGCTGGAACATGTGGCGTGCTGCTGCTGAGCCTGGTCATCACCC
TGTATTGCAAGAGAGGCCGGAAGAAGCTGCTCTACATCTTCAAGCAGCCCTTCATGAGGCCCGTGCAGACCACACA
AGAAGAGGACGGCTGCTCCTGCAGATTCCCCGAGGAAGAGGAAGGCGGCTGCGAGCTGAGAGTGAAGTTCAGCAGA
AGCGCTGACGCCCCTGCTTACCAGCAGGGACAGAACCAGCTGTACAACGAGCTGAACCTGGGGAGAAGAGAAGAGT
ACGACGTGCTGGACAAGCGGAGAGGCAGAGATCCTGAGATGGGCGGCAAGCCCAGAAGAAAGAACCCTCAAGAGGG
CCTGTATAATGAGCTGCAGAAAGACAAGATGGCCGAGGCCTACAGCGAGATCGGAATGAAGGGCGAGCGCAGAAGA
GGCAAGGGACACGATGGACTGTACCAGGGCCTGAGCACAGCCACCAAGGACACATATGACGCCCTGCACATGCAGG
CCCTGCCTCCAAGA (SEQ ID NO: 266)
miR-122 binding sequence (Bold underlined): CAAACACCATTGTCACACTCCA (SEQ ID
NO: 40)
miR-122_1 spacer: aaaaacacaaacCAAACACCATTGTCACACTCCAaaaaaaacaaaca (SEQ ID
NO: 270)
3' UTR (bold) with 3' exon fragment (lower case and underlined):
TAATAGaaaaacacaaacCAAACACCATTGTCACACTCCAaaaaaaacaaacaACCACACAAATGGTCGCCGAaac
tactgaaagcat (SEQ ID NO: 272)
3' exon fragment sequence is in lower case and is underlined
3' UTR:
TAATAGaaaaacacaaacCAAACACCATTGTCACACTCCAaaaaaaacaaacaACCACACAAATGGTCGCCGA
(SEQ ID NO: 350)
Encoded Anti-CD19 protein:
MALPVTALLLPLALLLHAARPDIQMTOTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR (SEQ ID NO: 174)
*Because the sequences are RNA sequences, it is understood that the T's represent uracil in the sequences of this table. Equivalent RNA sequences showing U's in place of T's are provided in Table 7C.

Table 7C depicts the RNA equivalent sequences for exemplary sequences provided in Table 7B. As used herein, the sequences in Table 7B and Table 7C are interchangeable and are understood to comprise uracils in the context of RNA sequences (e.g., circRNA sequences or precursor RNA sequences). Table 7C provides two exemplary circRNA sequences with an annotation of the specific combination of precisely engineered elements (5′ UTR, IRES, codon-optimized coding sequence encoding a protein sequence, and 3′ UTR (including a single microRNA binding site)). Table 7C also provides the amino acid sequence of the anti-CD19 antibody or antigen-binding fragment that is encoded by the codon-optimized coding sequence of the circRNA.

TABLE 7C
Exemplary circRNA sequences, and sequence for the encoded protein within the
circRNA

AACUUUUAGUUAUUUUCGGCGACCAUUUGUGUGGUGAGCUCUAAGUGUUGAUCAGCGGGCGCAGCUGCAUAUGACC
UCUGCGUGUGCCUGGUAAUUGACUAAGAUAUCUUACUCAGCGUAACUACUCCGGGUUACGUGAUGAAGAAGAGGCU
ACGGAGAUUCUCGGGCUACGGCCCUGGAGCCACUCCGGCUCCUAAAGAUUUAGAAGUUUGAGCACACCCGCCCACU
AGGGCCCCCCAUCCAGGGGGGCAACGGGCAAGCACUUCUGUUUCCCCGGUAUGAUCUGAUAGGCUGUAACCACGGC
UGAAACAGAGAUUAUCGUUAUCCGCUUCACUACUUCGAGAAGCCUAGUAAUGAUGGGUGAAAUUGAAUCCGUUGAU
CCGGUGUCUCCCCCACACCAGAAACUCAUGAUGAGGGUUGCCAUCCCGGCUACGGCGACGUAGCGGGCAUCCCUGC
GCUGGCAUGAGGCCUCUUAGGAGGACGGAUGAUAUGGAUCUUGUCGUGAAGAGCCUAUUGAGCUAGUGUCGACUCC
UCCGCCCCCGUGAAUGCGGCUAAUCCUAACCCCGGAGCAGGUGGGUCCAAUCCAGGGCCUGGCCUGUCGUAAUGCG
UAAGUCUGGGACGGAACCGACUACUUUCGGGAAGGCGUGUUUCCAUUUGUUCAUUAUUUGUGUGUUUAUGGUGACA
ACUCUGGGUAAACGUUCUAUUGCGUUUAUUGAGAGAUUCCCAACAAUUGAACAAACGAGAACUACCUGUUUUAUUA
AAUUUACACAGAGAAGAAUUACAAUGGCUCUGCCUGUGACAGCUCUGCUGCUGCCUCUGGCUCUGCUUCUGCAUGC
CGCCAGACCUGACAUCCAGAUGACCCAGACAACCAGCAGCCUGUCUGCCAGCCUGGGCGACAGAGUGACCAUCAGC
UGUAGAGCCAGCCAGGACAUCAGCAAGUACCUGAACUGGUAUCAGCAGAAACCCGACGGCACCGUGAAGCUGCUGA
UCUACCACACCAGCAGACUGCACAGCGGCGUGCCAUCCAGAUUUUCUGGCAGCGGCUCUGGCACCGACUACAGCCU
GACAAUCAGCAACCUGGAACAAGAGGAUAUCGCUACCUACUUCUGCCAGCAAGGCAACACCCUGCCUUACACAUUC
GGCGGAGGCACCAAGCUGGAAAUCACAGGUGGCGGAGGAUCUGGCGGAGGUGGAAGCGGAGGCGGUGGAUCUGAAG
UGAAACUGCAAGAGUCUGGCCCUGGCCUGGUGGCUCCAUCUCAGUCUCUGAGCGUGACCUGUACCGUCAGCGGAGU
GUCUCUGCCUGAUUACGGCGUGUCCUGGAUCAGACAGCCUCCUAGAAAAGGCCUGGAAUGGCUGGGCGUGAUCUGG
GGCAGCGAGACAACCUACUACAACAGCGCCCUGAAGUCCAGGCUGACCAUCAUCAAGGACAACUCCAAGAGCCAGG
UGUUCCUGAAGAUGAACAGCCUGCAGACCGACGACACCGCCAUCUACUACUGCGCCAAGCACUACUACUACGGCGG
CAGCUACGCCAUGGAUUACUGGGGCCAGGGAACCAGCGUGACCGUGUCUAGCACAACAACCCCUGCUCCUAGACCU
CCUACACCAGCUCCUACAAUCGCCAGCCAGCCUCUGUCUCUGAGGCCAGAGGCUUGUAGACCUGCUGCUGGCGGAG
CUGUGCACACAAGAGGCCUGGAUUUCGCCUGCGACAUCUACAUCUGGGCCCCUCUGGCUGGAACAUGUGGCGUGCU
GCUGCUGAGCCUGGUCAUCACCCUGUAUUGCAAGAGAGGCCGGAAGAAGCUGCUCUACAUCUUCAAGCAGCCCUUC
AUGAGGCCCGUGCAGACCACACAAGAAGAGGACGGCUGCUCCUGCAGAUUCCCCGAGGAAGAGGAAGGCGGCUGCG
AGCUGAGAGUGAAGUUCAGCAGAAGCGCUGACGCCCCUGCUUACCAGCAGGGACAGAACCAGCUGUACAACGAGCU
GAACCUGGGGAGAAGAGAAGAGUACGACGUGCUGGACAAGCGGAGAGGCAGAGAUCCUGAGAUGGGGGCAAGCCC
AGAAGAAAGAACCCUCAAGAGGGCCUGUAUAAUGAGCUGCAGAAAGACAAGAUGGCCGAGGCCUACAGCGAGAUCG
GAAUGAAGGGCGAGCGCAGAAGAGGCAAGGGACACGAUGGACUGUACCAGGGCCUGAGCACAGCCACCAAGGACAC
AUAUGACGCCCUGCACAUGCAGGCCCUGCCUCCAAGAUAAUAGaacaaacacaaacCAAACACCAUUGUCACACUC
CAaacaacaaacaaacaACCACACAAAUGGUCGCCGAaacuacugaaagcau (SEQ ID NO: 351)
SEQ ID NO: 351 is an RNA rendering, equivalent to SEQ ID NO: 264.
5' UTR (Italics) with 5' exon fragment:
AACUUUUAGUUAUUUUCGGCGACCAUUUGUGUGGUGAGCUCUAAGUGUUGAUCAGCGGGCGCAGCUGCAUAUGACC
UCUGCGUGUGCCUGGUAAUUGACUAAGAUAUC (SEQ ID NO: 352)
5' exon fragment sequence (Twort sequence) is underlined.
SEQ ID NO: 352 is an RNA rendering, equivalent to SEQ ID NO: 265.
5' UTR (Italics):
UCGGCGACCAUUUGUGUGGUGAGCUCUAAGUGUUGAUCAGCGGGCGCAGCUGCAUAUGACCUCUGCGUGUGCCUGG
UAAUUGACUAAGAUAUC (SEQ ID NO: 353)
SEQ ID NO: 353 is an RNA rendering, equivalent to SEQ ID NO: 348.
IRES263 (Bold Italics):
UUACUCAGCGUAACUACUCCGGGUUACGUGAUGAAGAAGAGGCUACGGAGAUUCUCGGGCUACGGCCCUGGAGCCA
CUCCGGCUCCUAAAGAUUUAGAAGUUUGAGCACACCCGCCCACUAGGGCCCCCCAUCCAGGGGGGCAACGGGCAAG
CACUUCUGUUUCCCCGGUAUGAUCUGAUAGGCUGUAACCACGGCUGAAACAGAGAUUAUCGUUAUCCGCUUCACUA
CUUCGAGAAGCCUAGUAAUGAUGGGUGAAAUUGAAUCCGUUGAUCCGGUGUCUCCCCCACACCAGAAACUCAUGAU
GAGGGUUGCCAUCCCGGCUACGGCGACGUAGCGGGCAUCCCUGCGCUGGCAUGAGGCCUCUUAGGAGGACGGAUGA
UAUGGAUCUUGUCGUGAAGAGCCUAUUGAGCUAGUGUCGACUCCUCCGCCCCCGUGAAUGCGGCUAAUCCUAACCC
CGGAGCAGGUGGGUCCAAUCCAGGGCCUGGCCUGUCGUAAUGCGUAAGUCUGGGACGGAACCGACUACUUUCGGGA
AGGCGUGUUUCCAUUUGUUCAUUAUUUGUGUGUUUAUGGUGACAACUCUGGGUAAACGUUCUAUUGCGUUUAUUGA
GAGAUUCCCAACAAUUGAACAAACGAGAACUACCUGUUUUAUUAAAUUUACACAGAGAAGAAUUACA (SEQ ID
NO: 354)
SEQ ID NO: 354 is an RNA rendering, equivalent to SEQ ID NO: 1.
Codon-optimized Coding Sequence:
AUGGCUCUGCCUGUGACAGCUCUGCUGCUGCCUCUGGCUCUGCUUCUGCAUGCCGCCAGACCUGACAUCCAGAUGA
CCCAGACAACCAGCAGCCUGUCUGCCAGCCUGGGCGACAGAGUGACCAUCAGCUGUAGAGCCAGCCAGGACAUCAG
CAAGUACCUGAACUGGUAUCAGCAGAAACCCGACGGCACCGUGAAGCUGCUGAUCUACCACACCAGCAGACUGCAC
AGCGGCGUGCCAUCCAGAUUUUCUGGCAGCGGCUCUGGCACCGACUACAGCCUGACAAUCAGCAACCUGGAACAAG
AGGAUAUCGCUACCUACUUCUGCCAGCAAGGCAACACCCUGCCUUACACAUUCGGCGGAGGCACCAAGCUGGAAAU
CACAGGUGGCGGAGGAUCUGGCGGAGGUGGAAGCGGAGGCGGUGGAUCUGAAGUGAAACUGCAAGAGUCUGGCCCU
GGCCUGGUGGCUCCAUCUCAGUCUCUGAGCGUGACCUGUACCGUCAGCGGAGUGUCUCUGCCUGAUUACGGCGUGU
CCUGGAUCAGACAGCCUCCUAGAAAAGGCCUGGAAUGGCUGGGCGUGAUCUGGGGCAGCGAGACAACCUACUACAA
CAGCGCCCUGAAGUCCAGGCUGACCAUCAUCAAGGACAACUCCAAGAGCCAGGUGUUCCUGAAGAUGAACAGCCUG
CAGACCGACGACACCGCCAUCUACUACUGCGCCAAGCACUACUACUACGGCGGCAGCUACGCCAUGGAUUACUGGG
GCCAGGGAACCAGCGUGACCGUGUCUAGCACAACAACCCCUGCUCCUAGACCUCCUACACCAGCUCCUACAAUCGC
CAGCCAGCCUCUGUCUCUGAGGCCAGAGGCUUGUAGACCUGCUGCUGGCGGAGCUGUGCACACAAGAGGCCUGGAU
UUCGCCUGCGACAUCUACAUCUGGGCCCCUCUGGCUGGAACAUGUGGCGUGCUGCUGCUGAGCCUGGUCAUCACCC
UGUAUUGCAAGAGAGGCCGGAAGAAGCUGCUCUACAUCUUCAAGCAGCCCUUCAUGAGGCCCGUGCAGACCACACA
AGAAGAGGACGGCUGCUCCUGCAGAUUCCCCGAGGAAGAGGAAGGCGGCUGCGAGCUGAGAGUGAAGUUCAGCAGA
AGCGCUGACGCCCCUGCUUACCAGCAGGGACAGAACCAGCUGUACAACGAGCUGAACCUGGGGAGAAGAGAAGAGU
ACGACGUGCUGGACAAGCGGAGAGGCAGAGAUCCUGAGAUGGGCGGCAAGCCCAGAAGAAAGAACCCUCAAGAGGG
CCUGUAUAAUGAGCUGCAGAAAGACAAGAUGGCCGAGGCCUACAGCGAGAUCGGAAUGAAGGGCGAGCGCAGAAGA
GGCAAGGGACACGAUGGACUGUACCAGGGCCUGAGCACAGCCACCAAGGACACAUAUGACGCCCUGCACAUGCAGG
CCCUGCCUCCAAGA (SEQ ID NO: 355)
SEQ ID NO: 355 is an RNA rendering, equivalent to SEQ ID NO: 266.
miR-122 binding sequence (Bold underlined): CAAACACCAUUGUCACACUCCA (SEQ ID
NO: 356)
SEQ ID NO: 356 is an RNA rendering, equivalent to SEQ ID NO: 40.
miR-122_2 spacer: aacaaacacaaacCAAACACCAUUGUCACACUCCAaacaacaaacaaaca (SEQ ID
NO: 357)
SEQ ID NO: 357 is an RNA rendering, equivalent to SEQ ID NO: 267.
3' UTR with 3' exon fragment:
UAAUAGaacaaacacaaacCAAACACCAUUGUCACACUCCAaacaacaaacaaacaACCACACAAAUGGUCGCCGAa
acuacugaaagcau (SEQ ID NO: 358)
3' exon fragment sequence (Twort sequence) in lower case is underlined.
SEQ ID NO: 358 is an RNA rendering, equivalent to SEQ ID NO: 268.
3' UTR:
UAAUAGaacaaacacaaacCAAACACCAUUGUCACACUCCAaacaacaaacaaacaACCACACAAAUGGUCGCCGA
(SEQ ID NO: 359)
SEQ ID NO: 359 is an RNA rendering, equivalent to SEQ ID NO: 349.
Encoded Anti-CD19 protein:
MALPVTALLLPLALLLHAARPDIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR (SEQ ID NO: 174)
AACUUUUAGUUAUUUUCGGCGACCAUUUGUGUGGUGAGCUCUAAGUGUUGAUCAGCGGGCGCAGCUGCAUAUGACC
UCUGCGUGUGCCUGGUAAUUGACUAAGAUAUCUUACUCAGCGUAACUACUCCGGGUUACGUGAUGAAGAAGAGGCU
ACGGAGAUUCUCGGGCUACGGCCCUGGAGCCACUCCGGCUCCUAAAGAUUUAGAAGUUUGAGCACACCCGCCCACU
AGGGCCCCCCAUCCAGGGGGGCAACGGGCAAGCACUUCUGUUUCCCCGGUAUGAUCUGAUAGGCUGUAACCACGGC
UGAAACAGAGAUUAUCGUUAUCCGCUUCACUACUUCGAGAAGCCUAGUAAUGAUGGGUGAAAUUGAAUCCGUUGAU
CCGGUGUCUCCCCCACACCAGAAACUCAUGAUGAGGGUUGCCAUCCCGGCUACGGCGACGUAGCGGGCAUCCCUGC
GCUGGCAUGAGGCCUCUUAGGAGGACGGAUGAUAUGGAUCUUGUCGUGAAGAGCCUAUUGAGCUAGUGUCGACUCC
UCCGCCCCCGUGAAUGCGGCUAAUCCUAACCCCGGAGCAGGUGGGUCCAAUCCAGGGCCUGGCCUGUCGUAAUGCG
UAAGUCUGGGACGGAACCGACUACUUUCGGGAAGGCGUGUUUCCAUUUGUUCAUUAUUUGUGUGUUUAUGGUGACA
ACUCUGGGUAAACGUUCUAUUGCGUUUAUUGAGAGAUUCCCAACAAUUGAACAAACGAGAACUACCUGUUUUAUUA
AAUUUACACAGAGAAGAAUUACAAUGGCUCUGCCUGUGACAGCUCUGCUGCUGCCUCUGGCUCUGCUUCUGCAUGC
CGCCAGACCUGACAUCCAGAUGACCCAGACAACCAGCAGCCUGUCUGCCAGCCUGGGCGACAGAGUGACCAUCAGC
UGUAGAGCCAGCCAGGACAUCAGCAAGUACCUGAACUGGUAUCAGCAGAAACCCGACGGCACCGUGAAGCUGCUGA
UCUACCACACCAGCAGACUGCACAGCGGCGUGCCAUCUAGAUUUUCUGGCAGCGGCUCUGGCACCGACUACAGCCU
GACAAUCAGCAACCUGGAACAAGAGGAUAUCGCUACCUACUUCUGCCAGCAAGGCAACACCCUGCCUUACACAUUC
GGCGGAGGCACCAAGCUGGAAAUCACAGGUGGCGGAGGAUCUGGCGGAGGUGGAAGCGGAGGCGGUGGAUCUGAAG
UGAAACUGCAAGAGUCUGGCCCUGGCCUGGUGGCUCCAUCUCAGUCUCUGAGCGUGACCUGUACCGUCAGCGGAGU
GUCUCUGCCUGAUUACGGCGUGUCCUGGAUCAGACAGCCUCCUAGAAAAGGCCUGGAAUGGCUGGGCGUGAUCUGG
GGCAGCGAGACAACCUACUACAACAGCGCCCUGAAGUCCAGGCUGACCAUCAUCAAGGACAACUCCAAGAGCCAGG
UGUUCCUGAAGAUGAACAGCCUGCAGACCGACGACACCGCCAUCUACUACUGCGCCAAGCACUACUACUACGGCGG
CAGCUACGCCAUGGAUUACUGGGGCCAGGGAACCAGCGUGACCGUGUCUAGCACAACAACCCCUGCUCCUAGACCU
CCUACACCAGCUCCUACAAUCGCCAGCCAGCCUCUGUCUCUGAGGCCAGAGGCUUGUAGACCUGCUGCUGGCGGAG
CUGUGCACACAAGAGGCCUGGAUUUCGCCUGCGACAUCUACAUCUGGGCCCCUCUGGCUGGAACAUGUGGCGUGCU
GCUGCUGAGCCUGGUCAUCACCCUGUAUUGCAAGAGAGGCCGGAAGAAGCUGCUCUACAUCUUCAAGCAGCCCUUC
AUGAGGCCCGUGCAGACCACACAAGAAGAGGACGGCUGCUCCUGCAGAUUCCCCGAGGAAGAGGAAGGCGGCUGCG
AGCUGAGAGUGAAGUUCAGCAGAAGCGCUGACGCCCCUGCUUACCAGCAGGGACAGAACCAGCUGUACAACGAGCU
GAACCUGGGGAGAAGAGAAGAGUACGACGUGCUGGACAAGCGGAGAGGCAGAGAUCCUGAGAUGGGCGGCAAGCCC
AGAAGAAAGAACCCUCAAGAGGGCCUGUAUAAUGAGCUGCAGAAAGACAAGAUGGCCGAGGCCUACAGCGAGAUCG
GAAUGAAGGGCGAGCGCAGAAGAGGCAAGGGACACGAUGGACUGUACCAGGGCCUGAGCACAGCCACCAAGGACAC
AUAUGACGCCCUGCACAUGCAGGCCCUGCCUCCAAGAUAAUAGaaaaacacaaacCAAACACCAUUGUCACACUCC
AaaaaaaacaaacaACCACACAAAUGGUCGCCGAaacuacugaaagcau (SEQ ID NO: 360)
SEQ ID NO: 360 is an RNA rendering, equivalent to SEQ ID NO: 269.
5' UTR (Italics) with 5' exon fragment:
AACUUUUAGUUAUUUUCGGCGACCAUUUGUGUGGUGAGCUCUAAGUGUUGAUCAGCGGGCGCAGCUGCAUAUGACC
UCUGCGUGUGCCUGGUAAUUGACUAAGAUAUC (SEQ ID NO: 352)
5' exon fragment sequence underlined
SEQ ID NO: 352 is an RNA rendering, equivalent to SEQ ID NO: 265.
5' UTR (Italics):
UCGGCGACCAUUUGUGUGGUGAGCUCUAAGUGUUGAUCAGCGGGCGCAGCUGCAUAUGACCUCUGCGUGUGCCUGG
UAAUUGACUAAGAUAUC (SEQ ID NO: 353)
SEQ ID NO: 353 is an RNA rendering, equivalent to SEQ ID NO: 348.
IRES263 (Bold Italics):
UUACUCAGCGUAACUACUCCGGGUUACGUGAUGAAGAAGAGGCUACGGAGAUUCUCGGGCUACGGCCCUGGAGCCA
CUCCGGCUCCUAAAGAUUUAGAAGUUUGAGCACACCCGCCCACUAGGGCCCCCCAUCCAGGGGGGCAACGGGCAAG
CACUUCUGUUUCCCCGGUAUGAUCUGAUAGGCUGUAACCACGGCUGAAACAGAGAUUAUCGUUAUCCGCUUCACUA
CUUCGAGAAGCCUAGUAAUGAUGGGUGAAAUUGAAUCCGUUGAUCCGGUGUCUCCCCCACACCAGAAACUCAUGAU
GAGGGUUGCCAUCCCGGCUACGGCGACGUAGCGGGCAUCCCUGCGCUGGCAUGAGGCCUCUUAGGAGGACGGAUGA
UAUGGAUCUUGUCGUGAAGAGCCUAUUGAGCUAGUGUCGACUCCUCCGCCCCCGUGAAUGCGGCUAAUCCUAACCC
CGGAGCAGGUGGGUCCAAUCCAGGGCCUGGCCUGUCGUAAUGCGUAAGUCUGGGACGGAACCGACUACUUUCGGGA
AGGCGUGUUUCCAUUUGUUCAUUAUUUGUGUGUUUAUGGUGACAACUCUGGGUAAACGUUCUAUUGCGUUUAUUGA
GAGAUUCCCAACAAUUGAACAAACGAGAACUACCUGUUUUAUUAAAUUUACACAGAGAAGAAUUACA (SEQ ID
NO: 354)
SEQ ID NO: 354 is an RNA rendering, equivalent to SEQ ID NO: 1.
Codon-optimized Coding Sequence:
AUGGCUCUGCCUGUGACAGCUCUGCUGCUGCCUCUGGCUCUGCUUCUGCAUGCCGCCAGACCUGACAUCCAGAUGA
CCCAGACAACCAGCAGCCUGUCUGCCAGCCUGGGCGACAGAGUGACCAUCAGCUGUAGAGCCAGCCAGGACAUCAG
CAAGUACCUGAACUGGUAUCAGCAGAAACCCGACGGCACCGUGAAGCUGCUGAUCUACCACACCAGCAGACUGCAC
AGCGGCGUGCCAUCUAGAUUUUCUGGCAGCGGCUCUGGCACCGACUACAGCCUGACAAUCAGCAACCUGGAACAAG
AGGAUAUCGCUACCUACUUCUGCCAGCAAGGCAACACCCUGCCUUACACAUUCGGCGGAGGCACCAAGCUGGAAAU
CACAGGUGGCGGAGGAUCUGGCGGAGGUGGAAGCGGAGGCGGUGGAUCUGAAGUGAAACUGCAAGAGUCUGGCCCU
GGCCUGGUGGCUCCAUCUCAGUCUCUGAGCGUGACCUGUACCGUCAGCGGAGUGUCUCUGCCUGAUUACGGCGUGU
CCUGGAUCAGACAGCCUCCUAGAAAAGGCCUGGAAUGGCUGGGCGUGAUCUGGGGCAGCGAGACAACCUACUACAA
CAGCGCCCUGAAGUCCAGGCUGACCAUCAUCAAGGACAACUCCAAGAGCCAGGUGUUCCUGAAGAUGAACAGCCUG
CAGACCGACGACACCGCCAUCUACUACUGCGCCAAGCACUACUACUACGGCGGCAGCUACGCCAUGGAUUACUGGG
GCCAGGGAACCAGCGUGACCGUGUCUAGCACAACAACCCCUGCUCCUAGACCUCCUACACCAGCUCCUACAAUCGC
CAGCCAGCCUCUGUCUCUGAGGCCAGAGGCUUGUAGACCUGCUGCUGGCGGAGCUGUGCACACAAGAGGCCUGGAU
UUCGCCUGCGACAUCUACAUCUGGGCCCCUCUGGCUGGAACAUGUGGCGUGCUGCUGCUGAGCCUGGUCAUCACCC
UGUAUUGCAAGAGAGGCCGGAAGAAGCUGCUCUACAUCUUCAAGCAGCCCUUCAUGAGGCCCGUGCAGACCACACA
AGAAGAGGACGGCUGCUCCUGCAGAUUCCCCGAGGAAGAGGAAGGCGGCUGCGAGCUGAGAGUGAAGUUCAGCAGA
AGCGCUGACGCCCCUGCUUACCAGCAGGGACAGAACCAGCUGUACAACGAGCUGAACCUGGGGAGAAGAGAAGAGU
ACGACGUGCUGGACAAGCGGAGAGGCAGAGAUCCUGAGAUGGGCGGCAAGCCCAGAAGAAAGAACCCUCAAGAGGG
CCUGUAUAAUGAGCUGCAGAAAGACAAGAUGGCCGAGGCCUACAGCGAGAUCGGAAUGAAGGGCGAGCGCAGAAGA
GGCAAGGGACACGAUGGACUGUACCAGGGCCUGAGCACAGCCACCAAGGACACAUAUGACGCCCUGCACAUGCAGG
CCCUGCCUCCAAGA (SEQ ID NO: 355)
SEQ ID NO: 355 is an RNA rendering, equivalent to SEQ ID NO: 266.
miR-122 binding sequence (Bold underlined): CAAACACCAUUGUCACACUCCA (SEQ ID
NO: 356)
SEQ ID NO: 356 is an RNA rendering, equivalent to SEQ ID NO: 40.
miR-122_1 spacer: aaaaacacaaacCAAACACCAUUGUCACACUCCAaaaaaaacaaaca (SEQ ID
NO: 361)
SEQ ID NO: 361 is an RNA rendering, equivalent to SEQ ID NO: 270.
3' UTR (bold) with 3' exon fragment (bold and underlined):
UAAUAGaaaaacacaaacCAAACACCAUUGUCACACUCCAaaaaaaacaaacaACCACACAAAUGGUCGCCGAaac
uacugaaagcau (SEQ ID NO: 362)
3' exon fragment sequence (Twort sequence) is in lower case and underlined
SEQ ID NO: 362 is an RNA rendering, equivalent to SEQ ID NO: 272.
3' UTR:
UAAUAGaaaaacacaaacCAAACACCAUUGUCACACUCCAaaaaaaacaaacaACCACACAAAUGGUCGCCGA
(SEQ ID NO: 363)
SEQ ID NO: 363 is an RNA rendering, equivalent to SEQ ID NO: 350.
Encoded Anti-CD19 protein:
MALPVTALLLPLALLLHAARPDIQMTQTTSSLSASLGDRVTISCRASQDISKYLNWYQQKPDGTVKLLIYHTSRLH
SGVPSRFSGSGSGTDYSLTISNLEQEDIATYFCQQGNTLPYTFGGGTKLEITGGGGSGGGGSGGGGSEVKLQESGP
GLVAPSQSLSVTCTVSGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSTTTPAPRPPTPAPTIASQPLSLRPEACRPAAGGAVHTRGLD
FACDIYIWAPLAGTCGVLLLSLVITLYCKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKMAEAYSEIGMKGERRR
GKGHDGLYQGLSTATKDTYDALHMQALPPR (SEQ ID NO: 174)

In some embodiments, the circRNA comprises a sequence having at least 90% identity to SEQ ID NO: 351. In some embodiments, the circRNA comprises a sequence having at least 95% identity to SEQ ID NO: 351. In some embodiments, the circRNA comprises SEQ ID NO: 351. In some embodiments, the circRNA sequence is at least 90% identical to SEQ ID NO: 351. In some embodiments, the circRNA sequence is at least 95% identical to SEQ ID NO: 351. In some embodiments, the circRNA sequence is SEQ ID NO: 351.

In some embodiments, the 5′ UTR comprises a sequence having at least 90% identity to SEQ ID NO: 352. In some embodiments, the 5′ UTR comprises a sequence having at least 95% identity to SEQ ID NO: 352. In some embodiments, the 5′ UTR comprises SEQ ID NO: 352.

In some embodiments, the 5′ UTR is at least 90% identical to SEQ ID NO: 352. In some embodiments, the 5′ UTR is at least 95% identical to SEQ ID NO: 352. In some embodiments, the 5′ UTR sequence is SEQ ID NO: 352.

In some embodiments, the 5′ UTR comprises a sequence with at least 90% identity to SEQ ID NO: 353. In some embodiments, the 5′ UTR comprises a sequence with at least 95% identity to SEQ ID NO: 353. In some embodiments, the 5′ UTR comprises SEQ ID NO: 353.

In some embodiments, the 5′ UTR sequence is at least 90% identical to SEQ ID NO: 353. In some embodiments, the 5′ UTR sequence is at least 95% identical to SEQ ID NO: 353. In some embodiments, the 5′ UTR sequence is SEQ ID NO: 353.

In some embodiments, the IRES comprises a sequence having at least 90% identity to SEQ ID NO: 354. In some embodiments, the IRES comprises a sequence having at least 95% identity to SEQ ID NO: 354. In some embodiments, the IRES comprises SEQ ID NO: 354.

In some embodiments, the IRES sequence is at least 90% identical to SEQ ID NO: 354. In some embodiments, the IRES sequence is at least 95% identical to SEQ ID NO: 354. In some embodiments, the IRES sequence is SEQ ID NO: 354.

In some embodiments, the codon-optimized coding sequence comprises a sequence having at least 90% identity to SEQ ID NO: 355. In some embodiments, the codon-optimized coding sequence comprises a sequence having at least 95% identity to SEQ ID NO: 355. In some embodiments, the codon-optimized coding sequence comprises SEQ ID NO: 355.

In some embodiments, the miR-122 binding sequence comprises SEQ ID NO: 356.

In some embodiments, the miR-122_2 spacer comprises SEQ ID NO: 357.

In some embodiments, the 3′ UTR comprises a sequence having at least 90% identity to SEQ ID NO: 358. In some embodiments, the 3′ UTR comprises a sequence having at least 95% identity to SEQ ID NO: 358. In some embodiments, the 3′ UTR comprises a sequence having 100% identity to SEQ ID NO: 358. In some embodiments, the 3′ UTR comprises SEQ ID NO: 358.

In some embodiments, the 3′ UTR comprises a sequence having at least 90% identity to SEQ ID NO: 359. In some embodiments, the 3′ UTR comprises a sequence having at least 95% identity to SEQ ID NO: 359. In some embodiments, the 3′ UTR comprises a sequence having 100% identity to SEQ ID NO: 359. In some embodiments, the 3′ UTR comprises SEQ ID NO: 359.

In some embodiments, the circRNA comprises a sequence having at least 90% identity to SEQ ID NO: 360. In some embodiments, the circRNA comprises a sequence having at least 95% identity to SEQ ID NO: 360. In some embodiments, the circRNA comprises a sequence having 100% identity to SEQ ID NO: 360. In some embodiments, the circRNA comprises a sequence of SEQ ID NO: 360.

In some embodiments, the 5′ UTR comprises a sequence having at least 90% identity to SEQ ID NO: 352. In some embodiments, the 5′ UTR comprises a sequence having at least 95% identity to SEQ ID NO: 352. In some embodiments, the 5′ UTR comprises a sequence having 100% identity to SEQ ID NO: 352. In some embodiments, the 5′ UTR comprises SEQ ID NO: 352.

In some embodiments, the 5′ UTR comprises a sequence having at least 90% identity to SEQ ID NO: 353. In some embodiments, the 5′ UTR comprises a sequence having at least 95% identity to SEQ ID NO: 353. In some embodiments, the 5′ UTR comprises a sequence having 100% identity to SEQ ID NO: 353. In some embodiments, the 5′ UTR comprises SEQ ID NO: 353.

In some embodiments, the miR-122_1 spacer comprises a sequence of SEQ ID NO: 361.

In some embodiments, the 3′ UTR comprises a sequence having at least 90% identity to SEQ ID NO: 362. In some embodiments, the 3′ UTR comprises a sequence having at least 95% identity to SEQ ID NO: 362. In some embodiments, the 3′ UTR comprises a sequence having 100% identity to SEQ ID NO: 362. In some embodiments, the 3′ UTR comprises SEQ ID NO: 362.

In some embodiments, the 3′ UTR comprises a sequence having at least 90% identity to SEQ ID NO: 363. In some embodiments, the 3′ UTR comprises a sequence having at least 95% identity to SEQ ID NO: 363. In some embodiments, the 3′ UTR comprises a sequence having 100% identity to SEQ ID NO: 363. In some embodiments, the 3′ UTR comprises SEQ ID NO: 363.

Table 7D describes the features of an exemplary circRNA sequence of the present disclosure (e.g. SEQ ID NO: 264 in Table 7B or corresponding SEQ ID NO: 351 in Table 7C). In some embodiments, the circRNA comprises components (e.g., any subset of, or all of the components) of SEQ ID NO: 264 . These components and sequences together with their origin, location and function are described in Table 7D.

TABLE 7D
Summary of Features in Exemplary CircRNA Sequence of SEQ ID NO: 264 or SEQ ID NO: 351

| | | Location | |
|---|---|---|---|
| | | (nt) and | |
| | | SEQ ID | |
| Feature | Origin | NO. | Description of the Feature, including Function |
| 5' Exon | Staphylococcus | 1-15 | Sequence residue resulting from the Group I intron back-splicing |
| Fragment | phage | SEQ ID | mechanism, exhibited by the ″Twort.ORF142″ sequence, which |
| | Twort | NO: 382 | covalently join the 5' and 3' splice sites to form a closed circle. |
| | | | The 5' exon fragment contains the A (nucleotide position 1) of the |
| | | | dinucleotide “U-A” splice junction. |
| 5' UTR | Synthetic | 16-108 | The 5' untranslated region (UTR) contains a spacer |
| | Sequence | SEQ ID | UAAGUGUUGAUCAGCGGGCGCAGCUGCAUAUGACCUCUGC |
| | | NO: 348 | GUGUGCCUGG (SEQ ID NO: 384) as described in Table 7B, Table |
| | | or SEQ | 7C and Table 13B to facilitate RNA circularization. |
| | | ID NO: | |
| | | 353 | |
| IRES | Apodemus | 109-783 | The type I-like internal ribosomal entry site (IRES) facilitates |
| | agragius | SEQ ID | cap-independent translation of the coding sequence by assembling |
| | picornavirus | NO: 1 or | the ribosome and various initiation factors at the start codon. |
| | | SEQ ID | |
| | | NO: 354 | |
| CDS | Synthetic | 784-2241 | The coding sequence (CDS) encodes the nucleotide sequence of the |
| | Sequence | SEQ ID | anti-CD19 CAR , beginning with a start codon (AUG). This sequence |
| | | NO: 266 | will be translated to the anti-human CD19 CAR amino acid sequence |
| | | or SEQ | in the target cell cytoplasm, e.g., T cell cytoplasm. |
| | | ID NO: | |
| | | 355 | |
| 3' UTR | Synthetic | 2242- | The 3' untranslated region (UTR) contains a dual stop codon |
| | Sequence | 2317 | UAAUAG (No SEQ ID assigned for less than 10 nucleotides) and a |
| | | SEQ ID | spacer sequence comprising a single microRNA-122-5p binding |
| | | NO: 349 | sequence CAAACACCATTGTCACACTCCA (SEQ ID NO: 40) to |
| | | or SEQ | suppress off-target expression in the liver. The microRNA- |
| | | ID NO: | 122binding sequence is flanked by an upstream spacer AACAAACACAAAC |
| | | 359 | (SEQ ID NO: 373) and a downstream spacer |
| | | | AACAACAAACAAACA (SEQ ID NO: 374), which facilitate |
| | | | endogenous miRNA-122 binding and promotes circularization of the |
| | | | precursor linear RNA. |
| 3' Exon | Staphylococcus | 2318- | Sequence residue resulting from the Group I intron self-splicing |
| Fragment | phage | 2332 | mechanism, exhibited by the “Twort.ORF142” sequence, which |
| | Twort | SEQ ID | covalently join the 5' and 3' splice sites to form a closed circle. |
| | | NO: 389 | The 3' exon fragment contains the U (nucleotide position 2332) of |
| | | | the dinucleotide “U-A” splice junction. |

In some embodiments, the 5′ exon fragment and 3′ exon fragment originating from the Twort intron may be depicted as below with the 5′ exon fragment encompassed with the 5′ UTR, and the 3′ exon fragment encompassed within the 3′ UTR (as shown in SEQ ID NO: 265 and SEQ ID NO: 352)

In preferred embodiments, the 5′ UTR does not comprise the 5′ exon fragment (as shown in SEQ ID NO: 348 and SEQ ID NO: 353, and FIG. 3A). It is understood that circular RNA may be generated by various means described herein, of which use of the Twort intron is but one non-limiting example. In preferred embodiments, the 3′ UTR does not comprise the 3′ exon fragment. It is understood that a circular RNA sequence when depicted in linear form may have various alternate and equivalent representations. For example, in some embodiments, SEQ ID NO: 351 may also be presented as SEQ ID NO: 364, where both the 5′ exon fragment and 3′ exon fragment are shown after the 3′ UTR. In some embodiments, the 5′ exon fragment and 3′ exon fragment may be shown before the 5′ UTR.

(SEQ ID NO: 364)
UCGGCGACCAUUUGUGUGGUGAGCUCUAAGUGUUGAUCAGCGGGCGCAGCUGCAUAUGACCUCUGCGUGUGCCUGGU
AAUUGACUAAGAUAUCUUACUCAGCGUAACUACUCCGGGUUACGUGAUGAAGAAGAGGCUACGGAGAUUCUCGGGCU
ACGGCCCUGGAGCCACUCCGGCUCCUAAAGAUUUAGAAGUUUGAGCACACCCGCCCACUAGGGCCCCCCAUCCAGGG
GGGCAACGGGCAAGCACUUCUGUUUCCCCGGUAUGAUCUGAUAGGCUGUAACCACGGCUGAAACAGAGAUUAUCGUU
AUCCGCUUCACUACUUCGAGAAGCCUAGUAAUGAUGGGUGAAAUUGAAUCCGUUGAUCCGGUGUCUCCCCCACACCA
GAAACUCAUGAUGAGGGUUGCCAUCCCGGCUACGGCGACGUAGCGGGCAUCCCUGCGCUGGCAUGAGGCCUCUUAGG
AGGACGGAUGAUAUGGAUCUUGUCGUGAAGAGCCUAUUGAGCUAGUGUCGACUCCUCCGCCCCCGUGAAUGCGGCUA
AUCCUAACCCCGGAGCAGGUGGGUCCAAUCCAGGGCCUGGCCUGUCGUAAUGCGUAAGUCUGGGACGGAACCGACUA
CUUUCGGGAAGGCGUGUUUCCAUUUGUUCAUUAUUUGUGUGUUUAUGGUGACAACUCUGGGUAAACGUUCUAUUGCG
UUUAUUGAGAGAUUCCCAACAAUUGAACAAACGAGAACUACCUGUUUUAUUAAAUUUACACAGAGAAGAAUUACAAU
GGCUCUGCCUGUGACAGCUCUGCUGCUGCCUCUGGCUCUGCUUCUGCAUGCCGCCAGACCUGACAUCCAGAUGACCC
AGACAACCAGCAGCCUGUCUGCCAGCCUGGGCGACAGAGUGACCAUCAGCUGUAGAGCCAGCCAGGACAUCAGCAAG
UACCUGAACUGGUAUCAGCAGAAACCCGACGGCACCGUGAAGCUGCUGAUCUACCACACCAGCAGACUGCACAGCGG
CGUGCCAUCCAGAUUUUCUGGCAGCGGCUCUGGCACCGACUACAGCCUGACAAUCAGCAACCUGGAACAAGAGGAUA
UCGCUACCUACUUCUGCCAGCAAGGCAACACCCUGCCUUACACAUUCGGCGGAGGCACCAAGCUGGAAAUCACAGGU
GGCGGAGGAUCUGGCGGAGGUGGAAGCGGAGGCGGUGGAUCUGAAGUGAAACUGCAAGAGUCUGGCCCUGGCCUGGU
GGCUCCAUCUCAGUCUCUGAGCGUGACCUGUACCGUCAGCGGAGUGUCUCUGCCUGAUUACGGCGUGUCCUGGAUCA
GACAGCCUCCUAGAAAAGGCCUGGAAUGGCUGGGCGUGAUCUGGGGCAGCGAGACAACCUACUACAACAGCGCCCUG
AAGUCCAGGCUGACCAUCAUCAAGGACAACUCCAAGAGCCAGGUGUUCCUGAAGAUGAACAGCCUGCAGACCGACGA
CACCGCCAUCUACUACUGCGCCAAGCACUACUACUACGGCGGCAGCUACGCCAUGGAUUACUGGGGCCAGGGAACCA
GCGUGACCGUGUCUAGCACAACAACCCCUGCUCCUAGACCUCCUACACCAGCUCCUACAAUCGCCAGCCAGCCUCUG
UCUCUGAGGCCAGAGGCUUGUAGACCUGCUGCUGGCGGAGCUGUGCACACAAGAGGCCUGGAUUUCGCCUGCGACAU
CUACAUCUGGGCCCCUCUGGCUGGAACAUGUGGCGUGCUGCUGCUGAGCCUGGUCAUCACCCUGUAUUGCAAGAGAG
GCCGGAAGAAGCUGCUCUACAUCUUCAAGCAGCCCUUCAUGAGGCCCGUGCAGACCACACAAGAAGAGGACGGCUGC
UCCUGCAGAUUCCCCGAGGAAGAGGAAGGCGGCUGCGAGCUGAGAGUGAAGUUCAGCAGAAGCGCUGACGCCCCUGC
UUACCAGCAGGGACAGAACCAGCUGUACAACGAGCUGAACCUGGGGAGAAGAGAAGAGUACGACGUGCUGGACAAGC
GGAGAGGCAGAGAUCCUGAGAUGGGCGGCAAGCCCAGAAGAAAGAACCCUCAAGAGGGCCUGUAUAAUGAGCUGCAG
AAAGACAAGAUGGCCGAGGCCUACAGCGAGAUCGGAAUGAAGGGCGAGCGCAGAAGAGGCAAGGGACACGAUGGACU
GUACCAGGGCCUGAGCACAGCCACCAAGGACACAUAUGACGCCCUGCACAUGCAGGCCCUGCCUCCAAGAUAAUAGa
acaaacacaaacCAAACACCAUUGUCACACUCCAaacaacaaacaaacaACCACACAAAUGGUCGCCGAaacuacug
aaagcauAACUUUUAGUUAUUU
The Twort-derived 5' and 3' exon fragment sequences are bold and underlined.
The bold underlined sequence in lower case is the 3' exon fragment, and the
bold underlined sequence in upper case is the 5' exon fragment in FIG. 3A.

Table 7E provides exemplary linear RNA constructs that were tested in comparison to certain circRNA constructs of the present disclosure, as described in more detail in the Examples.

TABLE 7E
Linear mRNA constructs

AGGAAATAAGAGAGAAAAGAAGAGTAAGAAGAAATATAAGAGCCACCATGCTGCTGCTGGTGACAAGCCTGCTGCT
GTGCGAGCTGCCCCACCCCGCCTTTCTGCTGATCCCCGACATCCAGATGACCCAGACCACCAGCAGCCTGAGCGCC
AGCCTGGGCGACAGAGTGACCATCAGCTGCCGGGCCAGCCAGGACATCAGCAAGTACCTGAACTGGTATCAGCAGA
AACCCGACGGCACCGTGAAGCTGCTGATCTACCACACCTCCCGGCTGCACAGCGGCGTGCCCAGCAGATTTTCTGG
CAGCGGCAGCGGCACCGACTACAGCCTGACCATCTCCAACCTGGAACAAGAAGATATCGCTACCTATTTTTGTCAA
CAAGGCAACACCCTGCCCTACACCTTCGGCGGAGGCACCAAACTGGAAATCACCGGCAGCACCAGCGGCTCCGGCA
AGCCTGGATCTGGCGAGGGCAGCACCAAGGGCGAAGTGAAGCTGCAAGAAAGCGGCCCTGGCCTGGTGGCCCCTAG
CCAGAGCCTGTCCGTGACCTGTACCGTGTCCGGCGTGTCCCTGCCCGACTACGGCGTGTCCTGGATCCGGCAGCCC
CCCAGAAAGGGCCTGGAATGGCTGGGCGTGATCTGGGGCAGCGAGACAACCTACTACAACAGCGCCCTGAAGTCCC
GGCTGACCATCATCAAGGACAACAGCAAGAGCCAGGTGTTCCTGAAGATGAACAGCCTGCAGACCGACGACACCGC
CATCTACTACTGCGCCAAGCACTACTACTACGGCGGCAGCTACGCCATGGACTACTGGGGCCAGGGCACCAGCGTG
ACCGTGTCCAGCTTCGTGCCCGTGTTTCTGCCCGCCAAGCCTACCACCACCCCTGCCCCTAGACCTCCCACCCCAG
CCCCAACAATCGCCAGCCAGCCTCTGTCCCTGCGGCCCGAAGCCTGTAGACCAGCTGCCGGCGGAGCCGTGCACAC
CAGAGGCCTGGACTTCGCCTGCGACATCTACATCTGGGCCCCTCTGGCCGGCACCTGTGGCGTGCTGCTGCTGAGC
CTGGTGATCACCCTGTACTGCAACCACCGGAACAGAAGCAAGCGGAGCCGGCTGCTGCACAGCGACTACATGAACA
TGACCCCAAGACGGCCTGGCCCCACCCGGAAGCACTACCAGCCTTACGCCCCTCCCAGAGACTTCGCCGCCTACCG
GTCCAGAGTGAAGTTCAGCAGATCCGCCGACGCCCCTGCCTACCAGCAGGGACAGAACCAGCTGTACAACGAGCTG
AACCTGGGCAGACGGGAAGAGTACGACGTGCTGGACAAGCGGAGAGGCCGGGACCCCGAGATGGGCGGAAAGCCCA
GACGGAAGAACCCCCAGGAAGGCCTGTATAACGAACTGCAGAAAGACAAGATGGCCGAGGCCTACAGCGAGATCGG
CATGAAGGGCGAGCGGAGGCGCGGCAAGGGCCACGATGGCCTGTACCAGGGCCTGAGCACCGCCACCAAGGACACC
TACGACGCCCTGCACATGCAGGCCCTGCCCCCCAGATGATAATAGGCTGGAGCCTCGGTGGCCATGCTTCTTGCCC
CTTGGGCCTCCCCCCAGCCCCTCCTCCCCTTCCTGCACCCGTACCCCCGTGGTCTTTGAATAAAGCCTGAGTAGGA
AGAAAAAAAAAAAAAAAAAAAAAAAAAAAAAAAAAAAAAAAAAAAAAAAAAAAAAAAAAAAAAAAAAAAAAAAAAA
AAAAAAAAAAAAAAAAAAAAAAAAAA (SEQ ID NO 274)
AGGAAATAAGAGAGAAAAGAAGAGTAAGAAGAAATATAAGAGCCACCATGGCTCTGCCTGTGACAGCTCTGCTGCT
GCCTCTGGCTCTGCTTCTGCATGCCGCCAGACCTGACATCCAGATGACCCAGACAACCAGCAGCCTGTCTGCCAGC
CTGGGCGACAGAGTGACCATCAGCTGTAGAGCCAGCCAGGACATCAGCAAGTACCTGAACTGGTATCAGCAGAAAC
CCGACGGCACCGTGAAGCTGCTGATCTACCACACCAGCAGACTGCACAGCGGCGTGCCATCTAGATTTTCTGGCAG
CGGCTCTGGCACCGACTACAGCCTGACAATCAGCAACCTGGAACAAGAGGATATCGCTACCTACTTCTGCCAGCAA
GGCAACACCCTGCCTTACACATTCGGCGGAGGCACCAAGCTGGAAATCACAGGTGGCGGAGGATCTGGCGGAGGTG
GAAGCGGAGGCGGTGGATCTGAAGTGAAACTGCAAGAGTCTGGCCCTGGCCTGGTGGCTCCATCTCAGTCTCTGAG
CGTGACCTGTACCGTCAGCGGAGTGTCTCTGCCTGATTACGGCGTGTCCTGGATCAGACAGCCTCCTAGAAAAGGC
CTGGAATGGCTGGGCGTGATCTGGGGCAGCGAGACAACCTACTACAACAGCGCCCTGAAGTCCAGGCTGACCATCA
TCAAGGACAACTCCAAGAGCCAGGTGTTCCTGAAGATGAACAGCCTGCAGACCGACGACACCGCCATCTACTACTG
CGCCAAGCACTACTACTACGGCGGCAGCTACGCCATGGATTACTGGGGCCAGGGAACCAGCGTGACCGTGTCTAGC
ACAACAACCCCTGCTCCTAGACCTCCTACACCAGCTCCTACAATCGCCAGCCAGCCTCTGTCTCTGAGGCCAGAGG
CTTGTAGACCTGCTGCTGGCGGAGCTGTGCACACAAGAGGCCTGGATTTCGCCTGCGACATCTACATCTGGGCCCC
TCTGGCTGGAACATGTGGCGTGCTGCTGCTGAGCCTGGTCATCACCCTGTATTGCAAGAGAGGCCGGAAGAAGCTG
CTCTACATCTTCAAGCAGCCCTTCATGAGGCCCGTGCAGACCACACAAGAAGAGGACGGCTGCTCCTGCAGATTCC
CCGAGGAAGAGGAAGGCGGCTGCGAGCTGAGAGTGAAGTTCAGCAGAAGCGCTGACGCCCCTGCTTACCAGCAGGG
ACAGAACCAGCTGTACAACGAGCTGAACCTGGGGAGAAGAGAAGAGTACGACGTGCTGGACAAGCGGAGAGGCAGA
GATCCTGAGATGGGCGGCAAGCCCAGAAGAAAGAACCCTCAAGAGGGCCTGTATAATGAGCTGCAGAAAGACAAGA
TGGCCGAGGCCTACAGCGAGATCGGAATGAAGGGCGAGCGCAGAAGAGGCAAGGGACACGATGGACTGTACCAGGG
CCTGAGCACAGCCACCAAGGACACATATGACGCCCTGCACATGCAGGCCCTGCCTCCAAGATGATAATAGGCTGGA
GCCTCGGTGGCCATGCTTCTTGCCCCTTGGGCCTCCCCCCAGCCCCTCCTCCCCTTCCTGCACCCGTACCCCCGTG
GTCTTTGAATAAAGCCTGAGTAGGAAGAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAA (SEQ ID NO: 275)
*Because the sequences are RNA sequences,
it is understood that the T's represent
uracil in the sequences of this table.

### Chimeric Antigen Receptor

In accordance with the present invention, provided herein is a circular ribonucleic acid (circRNA) for targeted delivery comprising: (i) a circRNA comprising a coding sequence encoding a chimeric antigen receptor (CAR) comprising an anti-CD19 antibody or antigen-binding fragment thereof, (ii) a targeting moiety, and (iii) a delivery vehicle, wherein the targeting moiety is conjugated to the delivery vehicle.

Chimeric antigen receptors (CARs or CAR-Ts) are genetically-engineered receptors. These engineered receptors may be expressed by immune cells, including T cells. With a CAR, a single receptor may be programmed to both recognize a specific antigen and, when bound to that antigen, activate the immune cell to attack and destroy the cell bearing that antigen. When these antigens exist on B cells, an immune cell (e.g., a T cell) that expresses the CAR may target and kill the B cell, thereby providing a method for B cell depletion in treating B cell mediated diseases (e.g., autoimmune diseases and cancer).

Although not bound by any mechanism, a targeting moiety, e.g., anti-CD5 nanobody of the present disclosure, allows specific targeting of the circRNA encapsulated in the LNP to the receptor, e.g., CD5 expressed on T cells. Thus, the circRNA encoding the anti-CD19 CAR of the present disclosure is delivered to the T cell, which uses the T cell's own machinery to translate the anti-CD19 CAR and express the CAR on the surface of the T cell. The CAR T cell then binds to its target, e.g., B cells expressing CD19, via the antigen-binding domain of the CAR. Antigen binding to the CAR triggers intracellular signaling domains, leading to T cell activation that induces secretion of cytokines and cytotoxic molecules. These molecules destroy and eliminate target B cells, resulting in B cell depletion, which is the intended on-target effect for the therapeutic treatment of B cell-mediated diseases. Such in vivo CAR therapy disclosed herein provides multiple benefits over current ex vivo CAR therapies, for which manufacturing is complex, laborious, and expensive. For example, ex vivo CAR therapies require patients' or donor's T cells to be isolated from peripheral blood via apheresis, activated, and subsequently transduced by a lentivirus comprising a chimeric antigen receptor (CAR) construct to create an engineered CAR T cell. CAR T cells are further expanded before infusing engineered T cells into a patient's bloodstream to attack target cells. Further, ex vivo therapy requires lymphodepletion prior to administration, and is also associated with serious side-effects including cytokine release syndrome (CRS), which can cause multi-organ failure and even death, and immune effector cell-associated neurotoxicity syndrome (ICANS), which is a neuropsychiatric syndrome that can cause confusion and seizures. In contrast, the in vivo CAR therapy of the present invention uses the body's own immune system to manufacture the CAR T cells in vivo and bypasses the complex manufacturing steps required for ex vivo therapy. In vivo CAR therapy also does not require a step of lymphodepletion prior to administration of the therapy to the patient.

In some embodiments, provided herein is a circRNA for targeted delivery comprising: (i) a circRNA comprising a coding sequence encoding a chimeric antigen receptor (CAR) comprising an anti-CD20 antibody or antigen-binding fragment thereof, (ii) a targeting moiety, and (iii) a delivery vehicle, wherein the targeting moiety is conjugated to the delivery vehicle. In some embodiments, the CAR encoded by the circRNA is a CAR comprising anti-CD20 antibody or antigen-binding fragment thereof. In some embodiments, the CAR comprising anti-CD20 antibody or antigen-binding fragment thereof is a surrogate for a CAR comprising anti-CD19 antibody or antigen-binding fragment thereof. Similar to CD19, CD20 is also a B cell marker expressed on the surface of various B cells. Both CD19 and CD20 play a role in B cell signaling and activation.

In some embodiments, provided herein is a circRNA, comprising a coding sequence encoding a chimeric antigen receptor (CAR) comprising an anti-CD19 antibody or antigen-binding fragment thereof, wherein the circRNA comprises an Internal Ribosome Entry Site (IRES) having at least 85% identity to a sequence comprising any one of SEQ ID NO: 1-11, and one or more untranslated region (UTR).

In some aspects, provided herein is a circular ribonucleic acid (circRNA) comprising (i) a coding sequence encoding a chimeric antigen receptor (CAR) comprising an anti-CD19 antibody or antigen-binding fragment thereof, (ii) an Internal Ribosome Entry Site (IRES) comprising a sequence having at least 85% identical to any one of SEQ ID NO: 1-11, 3′ and 5′ untranslated region(s) (UTR), and a single microRNA binding site.

In some embodiments, the coding sequence further encodes a CD8 signal peptide, and the CAR comprises a CD8 hinge, a CD8 transmembrane domain, a 4-1BB costimulatory domain, and a CD3 zeta signaling domain.

In some embodiments, the anti-CD19 antibody or antigen-binding fragment thereof comprises a VL sequence of SEQ ID NO: 54 and a VH sequence of SEQ ID NO: 55.

In some embodiments, the CAR comprises an anti-CD19 scFv, a CD8a hinge, a CD8a transmembrane domain, a 4-1BB costimulatory domain, and a CD3 zeta signaling domain.

In some embodiments, the coding sequence encodes an amino acid sequence of SEQ ID NO: 58.

In some embodiments, the CAR comprises an amino acid sequence of SEQ ID NO: 58.

In some embodiments, provided herein is a circRNA therapeutic comprising a circRNA of the present disclosure encapsulated in an LNP. In some embodiments, the LNP is conjugated to a targeting moiety, wherein the targeting moiety binds to T cells.

In some embodiments, provided herein is a circRNA therapeutic for targeted delivery, comprising: (i) a circRNA comprising a coding sequence encoding a chimeric antigen receptor (CAR) comprising an anti-CD19 antibody or antigen-binding fragment thereof, and (ii) an anti-CD5 T cell targeting moiety; wherein the circRNA is encapsulated in a lipid nanoparticle; and wherein the T cell targeting moiety is conjugated to the lipid nanoparticle.

In some embodiments, provided herein is a circRNA therapeutic for targeted delivery, comprising: (i) a circRNA comprising (a) a 5′ untranslated region (UTR), (b) an Internal Ribosome Entry Site (IRES) having at least 85% identity to SEQ ID NO: 1, (c) a coding sequence encoding: a CD8a signal peptide, and a Chimeric Antigen Receptor (CAR) comprising: an anti-CD19 scFv, a CD8a hinge, a CD8a transmembrane domain, a 4-1BB costimulatory domain, a CD3 zeta signaling domain, and (d) a 3′ UTR, and (ii) an anti-CD5 T cell targeting moiety comprising the complementarity determining region (CDR) sequences of GFTFSMYS (SEQ ID NO:64), ISTGARDT (SEQ ID NO: 65) and GDLRYGPDGYDY (SEQ ID NO: 66), wherein the circRNA is encapsulated in a lipid nanoparticle, and wherein the anti-CD5 T cell targeting moiety is conjugated to the lipid nanoparticle.

In some embodiments, provided herein is a circRNA therapeutic for targeted delivery, comprising: (i) a circRNA comprising (a) a 5′ untranslated region (UTR), (b) an Internal Ribosome Entry Site (IRES) having 100% identity to SEQ ID NO: 1, (c) a coding sequence encoding: a Chimeric Antigen Receptor (CAR) comprising: FMC63 anti-CD19 scFv having a VL sequence of DIQMTQTTSSLSASLGDRVTISCRASQDISKYLNWYQQKPDGTVKLLIYHTSRLHSGVPS RFSGSGSGTDYSLTISNLEQEDIATYFCQQGNTLPYTFGGGTKLEIT (SEQ ID NO: 54), linked by a linker having the amino acid sequence of GGGGSGGGGSGGGGS (SEQ ID NO: 56), and a VH sequence of EVKLQESGPGLVAPSQSLSVTCTVSGVSLPDYGVSWIRQ PPRKGLEWLGVIWGSETTYYNSALKSRLTIIKDNSKSQVFLKMNSLQTDDTAIYYCAKH YYYGGSYAMDYWGQGTSVTVSS (SEQ ID NO: 55), a CD8a hinge, a CD8a transmembrane domain, a 4-1BB costimulatory domain, a CD3 zeta signaling domain, and a CD8 signal peptide; and (d) a 3′ untranslated region (UTR) of SEQ ID NO: 46 comprising a miR-122 site, and (ii) an anti-CD5 T cell targeting moiety comprising the sequence of SEQ ID NO: 89, wherein the circRNA is encapsulated in a lipid nanoparticle, wherein the anti-CD5 T-cell targeting moiety is conjugated to the lipid nanoparticle via a thiol-maleimide conjugation. In some embodiments, the LNP comprises an ionizable lipid, a phospholipid, a sterol lipid, and one or more PEG-lipids.

In some embodiments, the CAR comprises the anti-CD19 antibody or antigen-binding fragment thereof, a transmembrane domain, a costimulatory domain, and one or more intracellular signaling domain.

In some embodiments, the CAR comprises the anti-CD19 antibody or antigen-binding fragment thereof, a transmembrane domain, a costimulatory domain, and one or more intracellular signaling domain.

### Antigen-Binding Domain of the CAR

The antigen-binding domain is a portion of the chimeric receptor which recognizes the antigen. In certain CAR architectures, the antigen-binding domain comprises: a single-chain variable fragment (scFv) derived from a monoclonal antibody. CARs have also been produced with domain antibody (dAb) or VHH antigen-binding domains. CARs are engineered to bind to an antigen (such as a cell-surface antigen) by incorporating an antigen-binding molecule that interacts with that targeted antigen. In some embodiments, the antigen-binding molecule is an antibody.

In some embodiments, the antibody or antigen-binding fragment thereof is a sdAb, (e.g., a VHH or VNAR antibody, or other sdAb (e.g., a variable domain of light chain only (VLL)), a single chain variable fragment (scFv), a Designed Ankyrin Repeat Protein (DARPin) or an affibody, or combinations thereof. In some embodiments, the antibody or antigen-binding fragment thereof is a single domain antibody (sdAb) or a single chain variable fragment (scFv). In some embodiments, the antibody or antigen binding fragment is a scFv. In some embodiments, the antibody or antigen binding fragment is a VHH.

### Anti-CD19 Antibody or Antigen-Binding Fragment Thereof

In some embodiments, the antibody or antigen-binding domain is an anti-CD19 antibody or antigen-binding fragment thereof.

In some embodiments, the anti-CD19 antibody or antigen-binding fragment thereof is an FMC63 single chain variable fragment (scFv). In some embodiments, the anti-CD19 antibody or antigen-binding fragment thereof comprises a VL sequence of SEQ ID NO: 54, and a VH sequence of SEQ ID NO: 55. In some embodiments, provided herein is a circRNA, wherein the anti-CD19 antibody or antigen-binding fragment comprises a VL sequence of SEQ ID NO: 54, and a VH sequence of SEQ ID NO: 55. In some embodiments, the VL and the VH are joined by a linker. In some embodiments, the linker comprises SEQ ID NO: 56.

In some embodiments, the anti-CD-19 antibody is another anti-CD19 antibody known in the art, including without limitation, SJ25C1 (Bejcek et al., Cancer Res., 1995), HD37 (Pezutto et al., J. Immunol. 138(9):2793-2799 (1987)), 4G7 (Meeker et al., Hybridoma, 1984), B43 (Bejcek, 1995), BLY3 (Bejcek, 1995), B4 (Freedman et al., 1987), B4 HB12b (Kansas & Tedder, J. Immunol., 1991); Yazawa et al., Proc. Natl. Acad. Sci. USA, 2005; Herbst et al., J. Pharmacol. Exp. Ther., 2010, BU12 (Gallard et al., J. Immunology, 1992), CLB-CD19 (De Rie, Cell. Immunol., 1989), GR37 and GR41 (Ganji et al, J Transl Med, 2023); SRB-37 and SRB-85 (Banihashemi et al, Iran J Basic Med Sci, 2018); h1218 (Zhang et al, Mol Cancer, 2023); 47G4 also called Hu19 (Alabanza et al, Mol Ther, 2017), and variants thereof, among others.

In some embodiments, the anti-CD19 antibody or antigen-binding fragment comprises a sequence having at least 90% identity to heavy chain complementarity determining region (CDR) sequences, CDR1 of SEQ ID NO: 48, CDR2 of SEQ ID NO: 49 and CDR3 of SEQ ID NO: 50, and a sequence having at least 90% identity to light chain CDR sequences, CDR1 of SEQ ID NO: 51, CDR2 of SEQ ID NO: 52 and CDR3 of SEQ ID NO: 53. In some embodiments, the anti-CD19 antibody or antigen-binding fragment comprises a sequence having at least 95% identity to heavy chain complementarity determining region (CDR) sequences, CDR1 of SEQ ID NO: 48, CDR2 of SEQ ID NO: 49 and CDR3 of SEQ ID NO: 50, and a sequence having at least 95% identity to light chain CDR sequences, CDR1 of SEQ ID NO: 51, CDR2 of SEQ ID NO: 52 and CDR3 of SEQ ID NO: 53.

In some embodiments, the anti-CD19 antibody or antigen-binding fragment comprises heavy chain complementarity determining region (CDR) sequences, CDR1, CDR2 and CDR3 of SEQ ID NOs: 48, 49 and 50, respectively, and light chain CDR sequences, CDR1, CDR2 and CDR3 of SEQ ID NOs: 51, 52 and 53, respectively.

In some embodiments, the anti-CD19 antibody or antigen-binding fragment comprises heavy chain complementarity determining region (CDR) sequences HCDR1, HCDR2 and HCDR3 of SEQ ID NOs: 48, 49 and 50 respectively, and light chain CDR sequences LCDR1, LCDR2, and LCDR3 of SEQ ID NOs: 51, 52 and 53, respectively. In some embodiments, the anti-CD19 antibody or antigen-binding fragment comprises heavy chain complementarity determining region (CDR) sequences HCDR1, HCDR2 and HCDR3 that collectively differ from the respective SEQ ID NOs: 48, 49 and 50 by no more than 10 amino acids (e.g., by no more than 5, 4, 3, or 2 amino acids), and light chain CDR sequences LCDR1, LCDR2, and LCDR3 that collectively differ from the respective SEQ ID NOs: 51, 52 and 53 by no more than 10 amino acids (e.g., by no more than 5, 4, 3, or 2 amino acids).

In some embodiments, the anti-CD19 antibody or antigen-binding fragment comprises a VL sequence having at least 85% identity to SEQ ID NO: 54, and a VH sequence having at least 85% identity to SEQ ID NO: 55. In some embodiments, the anti-CD19 antibody or antigen-binding fragment comprises a VL sequence having at least 90% identity to SEQ ID NO: 54, and a VH sequence having at least 90% identity to SEQ ID NO: 55. In some embodiments, the anti-CD19 antibody or antigen-binding fragment comprises a VL sequence having at least 95% identity to SEQ ID NO: 54, and a VH sequence having at least 95% identity to SEQ ID NO: 55. In some embodiments, the anti-CD19 antibody or antigen-binding fragment comprises a VL sequence having 100% identity to SEQ ID NO: 54, and a VH sequence having 100% identity to SEQ ID NO: 55. In some embodiments, the anti-CD19 antibody or antigen-binding fragment thereof comprises a VL sequence of SEQ ID NO: 54, and a VH sequence of SEQ ID NO: 55.

In some embodiments, the VL and the VH of the anti-CD19 antibody or antigen-binding fragment are joined by a linker. In some embodiments, the VL and VH of the anti-CD19 antibody or antigen-binding fragment are joined by a linker to form an scFv.

In some embodiments, the linker comprises a sequence having at least 80% identity to SEQ ID NO: 56. In some embodiments, the linker comprises a sequence having at least 85% identity to SEQ ID NO: 56. In some embodiments, the linker comprises a sequence having at least 90% identity to SEQ ID NO: 56. In some embodiments, the linker has 100% identity to the sequence of SEQ ID NO: 56.

In some embodiments, the anti-CD19 scFv comprises a VL sequence of

(SEQ ID NO: 54)
DIQMTQTTSSLSASLGDRVTISCRASQDISKYLNWYQQKPDGT
VKLLIYHTSRLHSGVPSRFSGSGSGTDYSLTISNLEQEDIATY
FCQQGNTLPYTFGGGTKLEIT,

linked by a linker having the amino acid sequence of GGGGSGGGGSGGGGS (SEQ ID NO: 56), and a VH sequence of

(SEQ ID NO: 55)
EVKLQESGPGLVAPSQSLSVTCTVSGVSLPDYGVSWIRQPPRKG
LEWLGVIWGSETTYYNSALKSRLTIIKDNSKSQVFLKMNSLQTD
DTAIYYCAKHYYYGGSYAMDYWGQGTSVTVSS.

TABLE 8
Exemplary Anti-CD19 Antibody or Antigen Binding Domain (FMC63;
VL-Linker-VH)

| Description | CDR Sequences (Chothia) | CDR Sequences (Kabat) |
|---|---|---|
| FMC63 | CDRH1: GVSLPDY (SEQ ID NO: 48) | CDRH1: DYGVS (SEQ ID NO: 261) |
| CDR | CDRH2: | CDRH2: VIWGSETTYYNSALKS (SEQ ID |
| Sequences | WGSET (SEQ ID NO: 49) | NO: 262) |
| | CDRH3: HYYYGGSYAMDY (SEQ ID NO: | CDRH3: HYYYGGSYAMDY (SEQ ID NO: |
| | 50) | 50) |
| | CDRL1: RASQDISKYLN (SEQ ID NO: | CDRL1: RASQDISKYLN (SEQ ID NO: |
| | 51) | 51) |
| | CDRL2: HTSRLHS (SEQ ID NO: 52) | CDRL2: HTSRLHS (SEQ ID NO: 52) |
| | CDRL3: QQGNTLPYT (SEQ ID NO: | CDRL3: QQGNTLPYT (SEQ ID NO: |
| | 53) | 53) |

| | |
|---|---|
| FMC63 VL | DIQMTQTTSSLSASLGDRVTISCRASQDISKYLNWYQQKPDGTVKLLIYHTSRLHSGVPSRESGS |
| Sequence | GSGTDYSLTISNLEQEDIATYFCQQGNTLPYTFGGGTKLEIT (SEQ ID NO: 54) |
| FMC63 VH | EVKLQESGPGLVAPSQSLSVTCTVSGVSLPDYGVSWIRQPPRKGLEWLGVIWGSETTYYNSALKS |
| Sequence | RLTIIKDNSKSQVFLKMNSLOTDDTAIYYCAKHYYYGGSYAMDYWGQGTSVTVSS (SEQ ID |
| | NO: 55) |
| (G4S)3 | GGGGSGGGGSGGGGS (SEQ ID NO: 56) |
| Linker | |
| Whitlow | GSTSGSGKPGSGEGSTKG (SEQ ID NO: 57) |
| Linker | |

Complementarity determining regions (CDRs) are defined based on Kabat numbering convention (Wu and Kabat, 1970 J. Exp. Med. 132:211-250) and Chothia numbering convention based on a structurally correct version of the Kabat convention. Corresponding sequences by IMGT (Lefranc, 1999 Nucleic Acids Research 27:209-212) or any other convention are also encompassed herein. Accordingly, in some embodiments, the anti-CD19 antibody or antigen-binding fragment comprises heavy chain complementarity determining region (CDR) sequences HCDR1, HCDR2 and HCDR3 from SEQ ID NO:55, and light chain CDR sequences LCDR1, LCDR2, and LCDR3 from SEQ ID NO:54.

### Signal Peptide of the CAR

In some embodiments, the coding sequence encoding the CAR comprises a sequence encoding a signal peptide. In some embodiments, the signal peptide is a CD8 peptide. In some embodiments, the CD8 peptide is a CD8a signaling peptide comprising SEQ ID NO: 282. In some embodiments, the signal peptide is a Colony Stimulating Factor 2 Receptor Subunit Alpha (CSF2RA) protein comprising SEQ ID NO: 283.

### Hinge/Spacer of the CAR

In some embodiments, the coding sequence encoding the CAR comprises a spacer sequence to connect the antigen-binding domain with the transmembrane domain and spatially separate the antigen-binding domain from the endodomain. A flexible spacer allows the antigen-binding domain to orient in different directions to facilitate binding. In some embodiments, the hinge is a CD8 hinge. In some embodiments, the CD8 hinge is a CD8a hinge comprising SEQ ID NO: 284 or SEQ ID NO: 285. In some embodiments, the CD8 hinge is a CD8a hinge comprising SEQ ID NO: 284. In some embodiments, the CD8 hinge is a CD8a hinge comprising SEQ ID NO: 285. In some embodiments, the hinge is a CD28 hinge comprising SEQ ID NO: 286.

### Transmembrane Domain of the CAR

The transmembrane domain is the portion of the chimeric antigen receptor that spans the cell membrane. The transmembrane domain may be any protein structure that is thermodynamically stable in a membrane. This is typically an alpha helix comprising of several hydrophobic residues. The transmembrane domain of any transmembrane protein can be used to supply the transmembrane portion of the chimeric receptor. In some embodiments, the transmembrane domain is a CD8 transmembrane domain comprising SEQ ID NO: 287. In some embodiments, the transmembrane domain is a CD8a transmembrane domain comprising SEQ ID NO: 287. In some embodiments, the transmembrane domain is a CD28 transmembrane domain comprising SEQ ID NO: 288. In some embodiments, the transmembrane domain is a TNFRSF19 transmembrane domain comprising SEQ ID NO: 289.

### Endodomain/Signaling Domain of the CAR

The endodomain or signaling domain is the signal-transmission portion of the chimeric receptor. It may be part of or associate with the intracellular domain of the chimeric receptor. This transmits an activation signal to the T cell after antigen is bound. These signals promote T-cell proliferation and survival. In some embodiments, the signaling domain is CD3 zeta comprising SEQ ID NO: 294. In some embodiments, the signaling domain is CD3 delta. In some embodiments, the signaling domain is CD3 epsilon. In some embodiments, the signaling domain is CD3 gamma.

### Costimulatory Domain of the CAR

In some embodiments, the CAR comprises a costimulatory domain, which provides for robust CAR T expansion, function, persistence, and activity. The costimulatory domain provides intracellular signaling domains e.g., 4-1BB or CD28 into the CAR. In some embodiments, the costimulatory domain is a 4-1BB costimulatory domain comprising SEQ ID NO: 292 or SEQ ID NO: 293. In some embodiments, the costimulatory domain is a 4-1BB costimulatory domain comprising SEQ ID NO: 292. In some embodiments, the costimulatory domain is a 4-1BB costimulatory domain comprising SEQ ID NO: 293. In some embodiments, the costimulatory domain is a CD28 costimulatory domain comprising SEQ ID NO: 291.

### Exemplary CAR Architectures

In some embodiments, the coding sequence comprises a sequence encoding a CD8 signal peptide, and a sequence encoding the CAR, the CAR comprising an anti-CD19 antibody or antigen-binding fragment thereof, a CD8 hinge, a CD8 transmembrane domain, a 4-1BB costimulatory domain, and a CD3 zeta signaling domain, comprising SEQ ID NO: 58.

In some embodiments, the CAR comprises an anti-CD19 scFv. In some embodiments, the coding sequence further encodes a CD8 signal peptide, and the CAR further comprises a CD8 hinge, a CD8 transmembrane domain, a 4-1BB costimulatory domain, and a CD3 zeta signaling domain. In some embodiments, the CAR comprises an anti-CD19 scFv, a CD8a hinge, a CD8a transmembrane domain, a 4-1BB costimulatory domain, and a CD3 zeta signaling domain. In some embodiments, the CAR comprises SEQ ID NO: 58.

In some embodiments, the coding sequence comprises a sequence encoding a CD8 signal peptide, and a sequence encoding the CAR, the CAR comprising the anti-CD19 antibody or antigen-binding fragment thereof, a CD28 hinge, a CD28 transmembrane, a CD28 costimulatory domain, and a CD3 zeta signaling domain, comprising SEQ ID NO: 59.

In some embodiments, the CAR comprises a sequence selected from any one of SEQ ID NO: 58-60 in Table 9A. In some embodiments, provided herein is a circRNA, wherein the coding sequence encodes the amino acid sequence of any one of SEQ ID NOs: 58-60.

In some embodiments, the CAR comprises an amino acid sequence of SEQ ID NO: 58.

In some embodiments, the CAR comprises an amino acid sequence of SEQ ID NO: 59.

In some embodiments, the CAR comprises an amino acid sequence of SEQ ID NO: 60.

In some embodiments, the coding sequence of the anti-CD19 antibody or antigen binding domain encodes an amino acid sequence having at least 80% identity to one of SEQ ID NOs: 58-60. In some embodiments, the coding sequence of the anti-CD19 antibody or antigen binding domain encodes an amino acid sequence having at least 85% identity to any one of SEQ ID NOs: 58-60. In some embodiments, the coding sequence of the anti-CD19 antibody or antigen binding domain encodes an amino acid sequence having at least 90% identity to any one of SEQ ID NOs: 58-60. In some embodiments, the coding sequence of the anti-CD19 antibody or antigen binding domain encodes an amino acid sequence having 100% identity to any one of SEQ ID NOs: 58-60. In some embodiments, the coding sequence encodes the amino acid sequence of any one of SEQ ID NOs: 58-60.

In some embodiments, the coding sequence encodes an amino acid sequence having at least 80% identity to SEQ ID NO: 58. In some embodiments, the coding sequence encodes an amino acid sequence having at least 85% identity to SEQ ID NO: 58. In some embodiments, the coding sequence encodes an amino acid sequence having at least 90% identity to SEQ ID NO: 58. In some embodiments, the coding sequence encodes an amino acid sequence having at least 95% identity to SEQ ID NO: 58. In some embodiments, the coding sequence encodes an amino acid sequence having at least 99% identity to SEQ ID NO: 58. In some embodiments, the coding sequence encodes an amino acid sequence of SEQ ID NO: 58.

In some embodiments, the coding sequence encodes an amino acid sequence having at least 80% identity to SEQ ID NO: 59. In some embodiments, the coding sequence encodes an amino acid sequence having at least 85% identity to SEQ ID NO: 59. In some embodiments, the coding sequence encodes an amino acid sequence having at least 90% identity to SEQ ID NO: 59. In some embodiments, the coding sequence encodes an amino acid sequence having at least 95% identity to SEQ ID NO: 59. In some embodiments, the coding sequence encodes an amino acid sequence having at least 99% identity to SEQ ID NO: 59. In some embodiments, the coding sequence encodes an amino acid sequence of SEQ ID NO: 59.

In some embodiments, the coding sequence encodes an amino acid sequence having at least 80% identity to SEQ ID NO: 60. In some embodiments, the coding sequence encodes an amino acid sequence having at least 85% identity to SEQ ID NO: 60. In some embodiments, the coding sequence encodes an amino acid sequence having at least 90% identity to SEQ ID NO: 60. In some embodiments, the coding sequence encodes an amino acid sequence having at least 95% identity to SEQ ID NO: 60. In some embodiments, the coding sequence encodes an amino acid sequence having at least 99% identity to SEQ ID NO: 60. In some embodiments, the coding sequence encodes an amino acid sequence of SEQ ID NO: 60.

Table 9A provides amino acid sequences for exemplary anti-CD19 CAR architectures that are encoded by the circRNAs of the present disclosure. Table 9B provides amino acid sequences for annotated components from Table 9A.

TABLE 9A
Exemplary Anti-CD19 Chimeric Antigen Receptor Architecture

CD8a Signal peptide-FMC63 scFv-CD8a spacer-CD8a Transmembrane domain
(TM)-4-1BB costimulatory domain-CD3z signaling domain
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITggggsggggsggggsEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKESRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR
(SEQ ID NO: 58)
Bold underlined: Human CD8a signal peptide
Italics: FMC63 antigen-binding domain (light chain)
Lower case: (G4S)3 Linker
Italics underlined: FMC63 antigen-binding domain (heavy chain)
Bold italics underlined: Human CD8 hinge domain
Bold Italics: Human CD8 Transmembrane domain
Underlined: Human CD8a cytoplasmic fragment
Normal: Human 4-1BB costimulatory domain
Bold: Human CD3zeta signaling domain
CD8a Signal peptide-FMC63 scFv-CD28 spacer-CD28 TM-CD28 costimulatory
domain-CD3z signaling domain
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITggggsggggsggggsEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSaaaIEVMYPPPYLDNEKSNGTIIHVKGKHLCPSPLFP
GPSKPFWVLVVVGGVLACYSLLVTVAFIIFWVRSKRSRLLHSDYMNMTPR
RPGPTRKHYQPYAPPRDFAAYRSRVKESRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR
(SEQ ID NO: 59)
Bold Underlined: Human CD8a signal peptide
Italics: FMC63 antigen-binding domain (light chain)
Lower Case: (G4S)3 Linker
Italics Underlined: FMC63 antigen-binding domain (heavy chain)
Lower Case Italics: Triple Alanine Linker
Bold Italics Underlined: Human CD28 hinge domain
Bold Italics: Human CD28 Transmembrane domain
Normal: Human CD28 costimulatory domain
Bold: Human CD3zeta signaling domain
CSF2RA Signal peptide-FMC63 scFv-CD8a extra spacer-CD8a spacer-CD8a
TM-CD28 costimulatory domain-CD3z signaling domain
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITgstsgsgkpgsgegstkgEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSfvpvflpakpTTTPAPRPPTPAPTIASQPLSLR
PEACRPAAGGAVHTRGLDFACDIYIWAPLAGTCGVLLLSLVITLYCNHRN
RSKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQAL
PPR
(SEQ ID NO: 60)
Bold Underlined: Human CSF2RA signal peptide
Italics: FMC63 antigen-binding domain (light chain)
Lower Case: Whitlow linker
Italics Underlined: FMC63 antigen-binding domain (heavy chain)
Lower Case Italics: CD8a extra spacer
Bold Italics Underlined: Human CD8a hinge domain (spacer)
Bold Italics: Human CD8a Transmembrane domain
Underlined: Human CD8a cytoplasmic long fragment
Normal: Human CD28 costimulatory domain
Bold: Human CD3zeta signaling domain
CSF2RA Signal peptide-FMC63 scFv-CD8a hinge-TNFRSF19 TM-41BB costimulatory
domain-CD3z signaling domain
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITggggsggggsggggsEVKLQE
SGPGLVAPSQSLSVTCTVSGVSLPDYGVSWIRQPPRKGLEWLGVIWGSET
TYYNSALKSRLTIIKDNSKSQVFLKMNSLQTDDTAIYYCAKHYYYGGSYA
MDYWGQGTSVTVSSaaaPAPRPPTPAPTIASQPLSLRPEACRPAAGGAVH
TRGLDFDTALAAVICSALATVLLALLILCVIYCKRQprrKKLLYIFKQPF
MRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR
(SEQ ID NO: 341)
BQld Underlined: Human CSF2RA signal peptide
Italics: FMC63 antigen-binding domain (light chain)
Lower Case: (G4S)3 Linker
Italics Underlined: FMC63 antigen-binding domain (heavy chain)
Lower Case Italics: Triple alanine linker
Bold Italics Underlined: Human CD8a hinge domain (spacer)
Bold Italics: Human TNFSRF19 Transmembrane domain
Lower Case Italics Underlined: PRR Linker
Normal: Human 4-1BB costimulatory domain Bold: Human CD3zeta signaling domain
CD8a Signal Peptide-FMC63 scFv-CD28 spacer-CD28 TM-CD28 costimulatory
domain-CD3z signaling domain
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITggggsggggsggggsEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSaaaIEVMYPPPYLDNEKSNGTIIHVKGKHLCPSPLFP
GPSKPFWVLVVVGGVLACYSLLVTVAFIIFWVRSKRSRLLHSDYMNMTPR
RPGPTRKHYQPYAPPRDFAAYRSRVKESRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR
(SEQ ID NO: 342)
Bold Underlined: Human CD8a signal peptide
Italics: FMC63 antigen-binding domain (light chain)
Lower Case: (G4S)3 Linker
Italics Underlined: FMC63 antigen-binding domain (heavy chain)
Lower Case Italics: Triple alanine linker
Bold Italics Underlined: Human CD28 hinge domain (spacer)
Bold Italics: Human CD28 Transmembrane domain
Normal: Human CD28 costimulatory domain
Bold: Human CD3zeta signaling domain

TABLE 9B
Elements of Exemplary CAR architecture in Table 9A

Exemplary Signal Peptides
Human CD8a signal peptide
MALPVTALLLPLALLLHAARP (SEQ ID NO: 282)
Human CSF2RA signal peptide
MLLLVTSLLLCELPHPAFLLIP (SEQ ID NO: 283)
FMC63 Antibody VL and VH
FMC63 antigen-binding domain (light chain)
DIQMTQTTSSLSASLGDRVTISCRASQDISKYLNWYQQKPDGTVKLLIYHTSRLHSGVPSRFSG
SGSGTDYSLTISNLEQEDIATYFCQQGNTLPYTFGGGTKLEIT (SEQ ID NO: 54)
FMC63 antigen-binding domain (heavy chain)
EVKLQESGPGLVAPSQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGTSVTVSS (SEQ ID NO: 55)
Linkers connecting FMC63 VL and VH
(G4S)3 Linker
GGGGSGGGGSGGGGS (SEQ ID NO: 56)
Whitlow Linker
GSTSGSGKPGSGEGSTKG (SEQ ID NO: 57)
Exemplary Hinge Domains
Human CD8a hinge domain (TTT-spacer-ACD)
TTTPAPRPPTPAPTIASQPLSLRPEACRPAAGGAVHTRGLDFACD (SEQ ID NO: 284)
Human CD8a hinge domain (spacer)
PAPRPPTPAPTIASQPLSLRPEACRPAAGGAVHTRGLDF
(SEQ ID NO: 285)
Human CD28 hinge domain (spacer)
IEVMYPPPYLDNEKSNGTIIHVKGKHLCPSPLFPGPSKPF (SEQ ID NO: 286)
Exemplary Transmembrane Domains
Human CD8 Transmembrane domain
IYIWAPLAGTCGVLLLSLVIT (SEQ ID NO: 287)
Human CD28 Transmembrane Domain
WVLVVVGGVLACYSLLVTVAFIIFWV (SEQ ID NO: 288)
Human TNFSRF19 Transmembrane Domain
DTALAAVICSALATVLLALLILCVIYCKRQ (SEQ ID NO: 289)
Exemplary Linkers/Spacers
Human CD8a cytoplasmic fragment
LYC
Triple Alanine Linker
AAA
CD8a Extra Spacer
FVPVFLPAKP
(SEQ ID NO: 290)
PRR Linker
PRR
Exemplary Costimulatory Domain
Human CD28 Costimulatory Domain
RSKRSRLLHSDYMNMTPRRPGPTRKHYQPYAPPRDFAAYRS (SEQ ID NO: 291)
Human 4-1BB costimulatory domain_1
KRGRKKLLYIFKQPFMRPVQTTQEEDGCSCRFPEEEEGGCEL (SEQ ID NO: 292)
Human 4-1BB costimulatory domain_2
KKLLYIFKQPFMRPVQTTQEEDGCSCRFPEEEEGGCEL (SEQ ID NO: 293)
Exemplary CD3zeta Signaling Domain
Human CD3zeta signaling domain
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR (SEQ ID NO: 294)

### Targeted Delivery Vehicle

Some embodiments of the disclosure relate to a targeted delivery vehicle for delivery of cargo (which may be, e.g., circRNA, or other cargo within the delivery vehicle), to an intended target, such as a T cell, using a targeting moiety.

### Targeting Moiety

In some embodiments, a targeting moiety is any moiety that binds or targets another moiety; a targeting moiety includes, for example, a peptide, polypeptide, oligopeptide, protein (such as antibody and antigen-binding fragment), small molecule, nucleic acid (including aptamers), vitamins, carbohydrates, or ligand, or combinations thereof. In some embodiments, a targeting moiety binds specifically to a target or multiple targets. In some embodiments, a targeting moiety includes any naturally occurring, synthetic, semi-synthetic, or recombinantly produced binding partner for a biological molecule or another target of interest. In some embodiments, the targeting moiety is a polypeptide comprising a binding domain and an N- or C-terminal extension comprising an accessible thiol group. In various embodiments, the targeting moiety of the LNP comprises an antibody or an antigen binding fragment of an antibody, an antigen, a ligand-binding domain of a receptor, or a receptor ligand. In some embodiments, the targeting moiety comprises an antibody or antigen binding fragment. In some embodiments, the targeting moiety comprises one or more of a complete antibody, an Fab, an F(ab′)2, an Fab′, a minibody, Fv, rIgG, a single chain Fv (scFv), a diabody, or a sdAb (e.g., VHH, VNAR). In some embodiments, a targeting moiety includes receptor ectodomains or a ligand-binding portions thereof, or ligands (e.g., cytokines, chemokines). In some embodiments, the targeting moiety comprises an sdAb (e.g., a VHH) or an scFv. In some embodiments, the targeting moiety comprises a VHH or an scFv. In some embodiments, the targeting moiety comprises a VHH.

In some embodiments, the receptor ligand is a carbohydrate, for example, a carbohydrate comprising terminal galactose or N-acetylgalactosamine units, which are bound by the asialoglycoprotein receptor.

In some embodiments, a targeting moiety dictates the localization and interaction of the RNA construct with a receptor, e.g., cell-surface receptors, to direct delivery of the RNA construct to particular on-target locations in a subject, such as specific organs, tissues, or cells. Targeting moieties are selected to recognize tissue-specific markers, organ-specific markers, or disease-specific markers (e.g., cell surface protein or epitope associated with a specific tissue, organ, or disease state, or a tumor marker).

In some embodiments, the targeting moiety is responsible for directing the delivery vehicle, e.g., lipid nanoparticles (LNPs) that encapsulate circRNAs, to interact with the designated effector cells.

In some embodiments, the targeting moiety binds to an immune cell.

In some embodiments, the targeting moiety binds to a cell surface marker on the immune cell. In some embodiments, the targeting moiety binds to a receptor on the cell surface of the immune cell. In some embodiments, the targeting moiety binds to an immune cell surface marker or receptor commonly known in the art. In some embodiments, the immune cell is, e.g., a T cell, a B cell, a natural killer (NK) cell, or a myeloid cell (e.g., macrophage, monocyte, dendric cell). In some embodiments, the immune cell is a T cell. In some embodiments, the immune cell is a B cell. In some embodiments, the immune cell is a natural killer (NK) cell.

In some embodiments, the targeting moiety binds to a cell surface marker on a T cell. Exemplary markers on T cells include, but are not limited to, TCRα, TCRβ, TCRγ, TCRζ, ICOS, CD28, CD27, HVEM, LIGHT, CD40, 4-1BB, OX40, DR3, GITR, TIM1, SLAM, CD2, CD3, CD4, CD5, CD6, CD7, CD8, and CD266. In some embodiments, the targeting moiety binds to a T cell surface marker or receptor commonly known in the art.

In some embodiments, the targeting moiety binds to a T cell. In some embodiments, the targeting moiety binds to a resting T cell. In some embodiments, the targeting moiety binds to an activated T cell. In some embodiments, the targeting moiety binds to a CD8 T cell. In some embodiments, the targeting moiety binds to a CD4 T cell.

In some embodiments, the targeting moiety binds to a cell surface marker on a B cell. Exemplary markers on B cells include, but are not limited to, BCR, CD5, CD19, CD20, CD21, CD22, CD23, CD24, CD37, CD38, CD40, CD40L, CD52, CD80, CD83, CD86, CD267, CD268, CD269, and HLA-DR. In some embodiments, the targeting moiety binds to aB cell surface marker or receptor commonly known in the art.

In some embodiments, the targeting moiety binds to a cell surface marker on an NK cell. Exemplary markers on NK cells include, but are not limited to, NKp30, NKp40, NKp44, NKp46, NKG2D, DNAM1, DAP10, CD16, CRTAM, CD27, PSGL1, CD96, CD100, NKp80, CD244, SLAMF6, SLAMF7, KIR2DS2, KIR3DS1, KIR2DS5, KIR2DS1, CD94, NKG2C, NKG2E, and CD160. In some embodiments, the targeting moiety binds to an NK cell surface marker or receptor commonly known in the art.

In preferred embodiments of the present disclosure, the targeting moiety binds specifically to T cells. In some embodiments, binding to T cells causes CAR expression and activation of T cells. CD69 is an early activation marker for T cells, i.e., it is one of the first surface proteins expressed on a T cell when it becomes activated by an antigen, rapidly appearing on the cell membrane shortly after T cell receptor (TCR) engagement, indicating T cell activation. In some embodiments, CD69 marker is increased upon anti-CD19 CAR expression after delivering circRNA formulated in a targeted LNP of the present disclosure.

### Targeting Antibody or Antigen-Binding Fragment Conjugated to the Delivery Vehicle

In some aspects, the circRNAs of the present disclosure are in a delivery vehicle that is conjugated to a targeting antibody or antigen-binding fragment thereof. In some embodiments, the targeting antibody or antigen-binding fragment thereof is conjugated to the surface of the engineered nanoparticle, such as a lipid nanoparticle.

In some embodiments, the targeting moiety comprises an antibody or an antigen-binding fragment. In some embodiments, the targeting moiety is selected from the group consisting of an anti-CD2, an anti-CD3, an anti-CD5, an anti-CD7, an anti-CD8 antibody or antigen-binding fragment thereof, and any combination thereof. In some embodiments, the targeting moiety is anti-CD5 and further comprises one or more of anti-CD2, an anti-CD3, an anti-CD5, an anti-CD7, an anti-CD8 antibody or antigen-binding fragment thereof, and combination thereof.

In some embodiments, the targeting moiety is one or more of an anti-CD2, an anti-CD3, an anti-CD5, an anti-CD7, or an anti-CD8 antibody or antigen-binding fragment thereof. In some embodiments, the targeting moiety is an anti-CD2 antibody or antigen-binding fragment thereof. In some embodiments, the targeting moiety is an anti-CD3 antibody or antigen-binding fragment thereof. In some embodiments, the targeting moiety is an anti-CD5 antibody or antigen-binding fragment thereof. In some embodiments, the targeting moiety is an anti-CD7 antibody or antigen-binding fragment thereof. In some embodiments, the targeting moiety is an anti-CD8 antibody or antigen-binding fragment thereof.

In some embodiments, the targeting moiety binds more than one target, wherein the targeting moiety comprises two or more of an anti-CD2, an anti-CD3, an anti-CD5, an anti-CD7, or an anti-CD8 antibody or antigen-binding fragment thereof in a single targeting moiety. In some embodiments, the targeting moiety comprises an anti-CD2 and an anti-CD3 antibody or antigen-binding fragment thereof in a single targeting moiety. In some embodiments, the targeting moiety comprises an anti-CD2 and an anti-CD5 antibody or antigen-binding fragment thereof in a single targeting moiety. In some embodiments, the targeting moiety comprises an anti-CD2 and an anti-CD7 antibody or antigen-binding fragment thereof in a single targeting moiety. In some embodiments, the targeting moiety comprises an anti-CD2 and an anti-CD8 antibody or antigen-binding fragment thereof in a single targeting moiety.

In some embodiments, the targeting moiety comprises an anti-CD3 and an anti-CD5 antibody or antigen-binding fragment thereof in a single targeting moiety. In some embodiments, the targeting moiety comprises an anti-CD3 and an anti-CD7 antibody or antigen-binding fragment thereof in a single targeting moiety. In some embodiments, the targeting moiety comprises an anti-CD3 and an anti-CD8 antibody or antigen-binding fragment thereof in a single targeting moiety.

In some embodiments, the targeting moiety comprises an anti-CD5 and an anti-CD7 antibody or antigen-binding fragment thereof in a single targeting moiety. In some embodiments, the targeting moiety comprises an anti-CD5 and an anti-CD8 antibody or antigen-binding fragment thereof in a single targeting moiety.

In some embodiments, the targeting moiety comprises an anti-CD7 and an anti-CD8 antibody or antigen-binding fragment thereof in a single targeting moiety.

In some embodiments, the antibody or antigen-binding fragment thereof is a single domain antibody (sdAb) (e.g., a VHH, a Variable New Antigen Receptor (VNAR), or VLL), a single chain variable fragment (scFv), a Designed Ankyrin Repeat Protein (DARPin) or an affibody, or combinations thereof. In some embodiments, the antibody or antigen-binding fragment thereof is a single domain antibody (sdAb). In some embodiments, the antibody or antigen-binding fragment thereof is a variable domain of heavy chain only antibody (VHH). In some embodiments, the antibody or antigen-binding fragment thereof is a Variable New Antigen Receptor (VNAR) fragment. In some embodiments, the antibody or antigen-binding fragment thereof is a variable domain of light chain only (VLL). In some embodiments, the antibody or antigen-binding fragment thereof is a single chain variable fragment (scFv). In some embodiments, the scFv comprises a heavy chain variable region (VH) and a light chain variable region (VL). In some embodiments, the antibody or antigen-binding fragment thereof is a Designed Ankyrin Repeat Protein (DARPin). In some embodiments, the antibody or antigen-binding fragment thereof is an affibody.

In some embodiments, the targeting antibody or antigen-binding fragment thereof comprises a Fab, a Fab′, a F(ab′)₂, an Fd, an Fc, a complementary determining region, a maxibody, a minibody, an intrabody, a diabody (e.g., bivalent and/or bispecific), a triabody, a tetrabody, or a chimeric (e.g., humanized) antibody. In some embodiments, the targeting antibody or antigen-binding fragment thereof comprises a Fab. In some embodiments, the targeting antibody or antigen-binding fragment thereof comprises a Fab′. In some embodiments, the targeting antibody or antigen-binding fragment thereof comprises a F(ab′)₂. In some embodiments, the targeting antibody or antigen-binding fragment thereof comprises an Fd. In some embodiments, the targeting antibody or antigen-binding fragment thereof comprises an Fc. In some embodiments, the targeting antibody or antigen-binding fragment thereof comprises a complementary determining region. In some embodiments, the targeting antibody or antigen-binding fragment thereof comprises a maxibody. In some embodiments, the targeting antibody or antigen-binding fragment thereof comprises a minibody. In some embodiments, the targeting antibody or antigen-binding fragment thereof comprises an intrabody. In some embodiments, the targeting antibody or antigen-binding fragment thereof comprises a diabody (e.g., bivalent and/or bispecific). In some embodiments, the targeting antibody or antigen-binding fragment thereof comprises a triabody. In some embodiments, the targeting antibody or antigen-binding fragment thereof comprises a tetrabody. In some embodiments, the targeting antibody or antigen-binding fragment thereof comprises a chimeric (e.g., humanized) antibody.

In some embodiments, the targeting moiety is a variable domain of heavy chain only (VHH) sequence.

In some embodiments, the targeting moiety is an anti-CD5 antibody or antigen-binding fragment thereof.

In some embodiments, the anti-CD5 antibody or antigen-binding fragment is a VHH.

In some embodiments, the targeting moiety is an anti-CD5 nanobody.

In some embodiments, the targeting moiety is an anti-CD5 antibody or antigen-binding fragment thereof disclosed in WO 2025/038813 entitled “CD5-Binding Polypeptides Comprising the Same, and Methods For Use Thereof”, which is incorporated by reference herein in its entirety. In some embodiments, the targeting moiety is an anti-CD5 antibody or antigen-binding fragment thereof comprising a set of CDR1, CDR2 and CDR3 sequences with at least 80%, at least 85%, at least 90%, at least 95% or at least 99% identity to a set of complementarity determining region (CDR) sequences (e.g., as designated in Chothia numbering), selected from any one of (a)-(i): (a) GRTFGTYT (SEQ ID NO: 61), ISWSAGRT (SEQ ID NO: 62) and DPWTSDSDYDRLTMYDY (SEQ ID NO: 63); (b) GFTFSMYS (SEQ ID NO: 64), ISTGARDT (SEQ ID NO: 65) and GDLRYGPDGYDY (SEQ ID NO: 66); (c) GSDFLVDA (SEQ ID NO: 67); MDIGGVT (SEQ ID NO: 68) and RGL (SEQ ID NO: 69); (d) GSDASTNN (SEQ ID NO: 70); IRTGGST (SEQ ID NO: 71) and WTGSPGALSDY (SEQ ID NO: 72); (e) GRTFINYA (SEQ ID NO: 73); ISRSGGRT (SEQ ID NO: 74) and ATVWEFTDGADQYDY (SEQ ID NO: 75); (f) GGTFSTYG (SEQ ID NO: 76); ITGSGVGT (SEQ ID NO: 77) and GHRPGWAVIRADAYEY (SEQ ID NO: 78); (g) GFSFSRVG (SEQ ID NO: 79); ISSDGSRT (SEQ ID NO: 80) and PGNS (SEQ ID NO: 81); (h) GFPFSSSF (SEQ ID NO: 82); IYSDGST (SEQ ID NO: 83) and VTGSI (SEQ ID NO: 84); (i) GRTTYN (SEQ ID NO: 85); IDLYGRAT (SEQ ID NO: 86) and DTSLPLGVLTESQRLYGA (SEQ ID NO: 87); or wherein the targeting moiety is an anti-CD5 antibody or antigen-binding fragment thereof comprising a set of CDR1, CDR2 and CDR3 sequences with at least 80%, at least 85%, at least 90%, at least 95%, or at least 99% identity to complementarity determining region (CDR) sequences (e.g., as designated in Kabat numbering), selected from any one of (j)-(r): (j) TYTMG (SEQ ID NO: 234); AISWSAGRTYYADSMKG (SEQ ID NO: 235) and DPWTSDSDYDRLTMYDY (SEQ ID NO: 236); (k) MYSMS (SEQ ID NO: 237); SISTGARDTAYADSVKG (SEQ ID NO: 238) and GDLRYGPDGYDY (SEQ ID NO: 239); (1) VDATT (SEQ ID NO: 240); IMDIGGVTEYADSVKG (SEQ ID NO: 241) and RGL (SEQ ID NO: 242); (m) TNNMA (SEQ ID NO: 243); LIRTGGSTHVADSMKG (SEQ ID NO: 244) and WTGSPGALSDY (SEQ ID NO: 245); (n) NYAAG (SEQ ID NO: 246); RISRSGGRTDYADSVKG (SEQ ID NO: 247); ATVWEFTDGADQYDY (SEQ ID NO: 248); (o) TYGMG (SEQ ID NO: 249); VITGSGVGTQYADSVKD (SEQ ID NO: 250) and GHRPGWAVIRADAYEY (SEQ ID NO: 251); (p) RVGMN (SEQ ID NO: 252); SISSDGSRTNYAHFVKG (SEQ ID NO: 253) and PGNS (SEQ ID NO: 254); (q) SSFMS (SEQ ID NO: 255); TIYSDGSTYYADSVKG (SEQ ID NO: 256) and VTGSI (SEQ ID NO: 257); (r) TYNMG (SEQ ID NO: 258); AIDLYGRATRYANSVKG (SEQ ID NO: 259); DTSLPLGVLTESQRLYGA (SEQ ID NO: 260).

In some embodiments, the targeting moiety is an anti-CD5 antibody or antigen-binding fragment thereof comprising complementarity determining region (CDR) sequences CDR1, CDR2 and CDR3 (e.g., as designated in Chothia numbering), selected from any one of (a)-(i): (a) GRTFGTYT (SEQ ID NO: 61), ISWSAGRT (SEQ ID NO: 62) and DPWTSDSDYDRLTMYDY (SEQ ID NO: 63); (b) GFTFSMYS (SEQ ID NO: 64), ISTGARDT (SEQ ID NO: 65) and GDLRYGPDGYDY (SEQ ID NO: 66); (c) GSDFLVDA (SEQ ID NO: 67); MDIGGVT (SEQ ID NO: 68) and RGL (SEQ ID NO: 69); (d) GSDASTNN (SEQ ID NO: 70); IRTGGST (SEQ ID NO: 71) and WTGSPGALSDY (SEQ ID NO: 72); (e) GRTFINYA (SEQ ID NO: 73); ISRSGGRT (SEQ ID NO: 74) and ATVWEFTDGADQYDY (SEQ ID NO: 75); (f) GGTFSTYG (SEQ ID NO: 76); ITGSGVGT (SEQ ID NO: 77) and GHRPGWAVIRADAYEY (SEQ ID NO: 78); (g) GFSFSRVG (SEQ ID NO: 79); ISSDGSRT (SEQ ID NO: 80) and PGNS (SEQ ID NO: 81); (h) GFPFSSSF (SEQ ID NO: 82); IYSDGST (SEQ ID NO: 83) and VTGSI (SEQ ID NO: 84); (i) GRTTYN (SEQ ID NO: 85); IDLYGRAT (SEQ ID NO: 86) and DTSLPLGVLTESQRLYGA (SEQ ID NO: 87); or wherein the targeting moiety is an anti-CD5 antibody or antigen-binding fragment thereof comprising complementarity determining region (CDR) sequences CDR1, CDR2 and CDR3 (e.g., as designated in Kabat numbering), selected from any one of (j)-(r): (j) TYTMG (SEQ ID NO: 234); AISWSAGRTYYADSMKG (SEQ ID NO: 235) and DPWTSDSDYDRLTMYDY (SEQ ID NO: 236); (k) MYSMS (SEQ ID NO: 237); SISTGARDTAYADSVKG (SEQ ID NO: 238) and GDLRYGPDGYDY (SEQ ID NO: 239); (1) VDATT (SEQ ID NO: 240); IMDIGGVTEYADSVKG (SEQ ID NO: 241) and RGL (SEQ ID NO: 242); (m) TNNMA (SEQ ID NO: 243); LIRTGGSTHVADSMKG (SEQ ID NO: 244) and WTGSPGALSDY (SEQ ID NO: 245); (n) NYAAG (SEQ ID NO: 246); RISRSGGRTDYADSVKG (SEQ ID NO: 247); ATVWEFTDGADQYDY (SEQ ID NO: 248); (o) TYGMG (SEQ ID NO: 249); VITGSGVGTQYADSVKD (SEQ ID NO: 250) and GHRPGWAVIRADAYEY (SEQ ID NO: 251); (p) RVGMN (SEQ ID NO: 252); SISSDGSRTNYAHFVKG (SEQ ID NO: 253) and PGNS (SEQ ID NO: 254); (q) SSFMS (SEQ ID NO: 255); TIYSDGSTYYADSVKG (SEQ ID NO: 256) and VTGSI (SEQ ID NO: 257); (r) TYNMG (SEQ ID NO: 258); AIDLYGRATRYANSVKG (SEQ ID NO: 259); DTSLPLGVLTESQRLYGA (SEQ ID NO: 260).

In some aspects, provided herein is a circular ribonucleic acid (circRNA) therapeutic for targeted delivery comprising: (i) a circRNA comprising a ribosome recruiting element, and a coding sequence encoding a chimeric antigen receptor (CAR) comprising an anti-CD19 antibody or antigen-binding fragment thereof, and (ii) a targeting moiety, and wherein the circRNA is encapsulated in a delivery vehicle, and wherein the targeting moiety is conjugated to the delivery vehicle, and wherein the targeting moiety comprises an anti-CD5 antibody heavy chain variable region comprising complementarity determining region (CDR) sequences CDR1, CDR2, and CDR3, wherein the CDR1, the CDR2, and the CDR3 are selected from any one of (a)-(i): (a) a CDR1 having an amino acid sequence of GRTFGTYT (SEQ ID NO: 61), a CDR2 having an amino acid sequence of ISWSAGRT (SEQ ID NO: 62), and a CDR3 having an amino acid sequence of DPWTSDSDYDRLTMYDY (SEQ ID NO: 63); (b) a CDR1 having an amino acid sequence of GFTFSMYS (SEQ ID NO: 64), a CDR2 having an amino acid sequence of ISTGARDT (SEQ ID NO: 65), and a CDR3 GDLRYGPDGYDY (SEQ ID NO: 66); (c) a CDR1 having an amino acid sequence of GSDFLVDA (SEQ ID NO: 67), a CDR2 having an amino acid sequence of MDIGGVT (SEQ ID NO: 68), and a CDR3 having an amino acid sequence of RGL (SEQ ID NO: 69); (d) a CDR1 having an amino acid sequence of GSDASTNN (SEQ ID NO: 70), a CDR2 having an amino acid sequence of IRTGGST (SEQ ID NO: 71), and a CDR3 having an amino acid sequence of WTGSPGALSDY (SEQ ID NO: 72); (e) a CDR1 having an amino acid sequence of GRTFINYA (SEQ ID NO: 73); a CDR2 having an amino acid sequence of ISRSGGRT (SEQ ID NO: 74), and a CDR3 having an amino acid sequence of ATVWEFTDGADQYDY (SEQ ID NO: 75); (f) a CDR1 having an amino acid sequence of GGTFSTYG (SEQ ID NO: 76), a CDR2 having an amino acid sequence of ITGSGVGT (SEQ ID NO: 77), and a CDR3 having an amino acid sequence of GHRPGWAVIRADAYEY (SEQ ID NO: 78); (g) a CDR1 having an amino acid sequence of GFSFSRVG (SEQ ID NO: 79), a CDR2 having an amino acid sequence of ISSDGSRT (SEQ ID NO: 80), and a CDR3 having an amino acid sequence of PGNS (SEQ ID NO: 81); (h) a CDR1 having an amino acid sequence of GFPFSSSF (SEQ ID NO: 82), a CDR2 having an amino acid sequence of IYSDGST (SEQ ID NO: 83), and a CDR3 having an amino acid sequence of VTGSI (SEQ ID NO: 84); or (i) a CDR1 having an amino acid sequence of GRTTYN (SEQ ID NO: 85), a CDR2 having an amino acid sequence of IDLYGRAT (SEQ ID NO: 86), and a CDR3 having an amino acid sequence of DTSLPLGVLTESQRLYGA (SEQ ID NO: 87).

In some embodiments, the anti-CD5 antibody VHH comprises the CDR1, CDR2, and CDR3 sequences GFTFSMYS (SEQ ID NO: 64), ISTGARDT (SEQ ID NO: 65), and GDLRYGPDGYDY (SEQ ID NO: 66), respectively.

In some aspects, provided herein is a circular ribonucleic acid (circRNA) therapeutic for targeted delivery comprising: (i) a circRNA comprising a ribosome recruiting element, and a coding sequence encoding a chimeric antigen receptor (CAR) comprising an anti-CD19 antibody or antigen-binding fragment thereof, and (ii) a targeting moiety, and wherein the circRNA is encapsulated in a delivery vehicle, and wherein the targeting moiety is conjugated to the delivery vehicle, and wherein the targeting moiety comprises an anti-CD5 antibody heavy chain variable region comprising CDR1, CDR2, and CDR3 sequences from SEQ ID NO: 89. In some embodiments, the CDR1, CDR2 and CDR3 sequences are SEQ ID NO: 64, SEQ ID NO: 65 and SEQ ID NO: 66 respectively from SEQ ID NO: 89 (e.g., as designated in Chothia numbering). In other embodiments, the CDR1, CDR2, and CDR3 sequences are SEQ ID NO: 237, SEQ ID NO: 238 and SEQ ID NO: 239 respectively from SEQ ID NO: 89 (e.g., as designated in Kabat numbering).

In some embodiments, the anti-CD5 antibody or antigen-binding fragment comprises a sequence having at least 75% identity to SEQ ID NO: 89. In some embodiments, the anti-CD5 antibody or antigen-binding fragment comprises a sequence having at least 80% identity to SEQ ID NO: 89. In some embodiments, the anti-CD5 antibody or antigen-binding fragment comprises a sequence having at least 85% identity to SEQ ID NO: 89. In some embodiments, the anti-CD5 antibody or antigen-binding fragment comprises a sequence having at least 90% identity to SEQ ID NO: 89. In some embodiments, the anti-CD5 antibody or antigen-binding fragment comprises a sequence having at least 95% identity to SEQ ID NO: 89. In some embodiments, the anti-CD5 antibody or antigen-binding fragment comprises SEQ ID NO: 89.

In some embodiments, the sequence of the anti-CD5 antibody or antigen-binding fragment is at least 75% identical to SEQ ID NO: 89. In some embodiments, the sequence of the anti-CD5 antibody or antigen-binding fragment is at least 80% identical to SEQ ID NO: 89. In some embodiments, the sequence of the anti-CD5 antibody or antigen-binding fragment is at least 85% identical to SEQ ID NO: 89. In some embodiments, the sequence of the anti-CD5 antibody or antigen-binding fragment is at least 90% identical to SEQ ID NO: 89. In some embodiments, the sequence of the anti-CD5 antibody or antigen-binding fragment is at least 95% identical to SEQ ID NO: 89. In some embodiments, the sequence of the anti-CD5 antibody or antigen-binding fragment is identical to SEQ ID NO: 89.

In some embodiments, the CDR1 sequence comprises SEQ ID NO: 64, the CDR2 sequence comprises SEQ ID NO: 65, and the CDR3 sequence comprises SEQ ID NO: 66.

In some embodiments, the delivery vehicle is a lipid nanoparticle (LNP).

In some embodiments, the targeting moiety binds more than one target.

In some embodiments, the anti-CD5 antibody or antigen-binding fragment is an anti-CD5 VHH. In some embodiments, the anti-CD5 antibody VHH comprises SEQ ID NO: 89.

In some embodiments, the VHH has a length of 110 to 130 (e.g., 115 to 125) amino acids. In some embodiments, the VHH has a length of 110 to 130 amino acids. In some embodiments, the VHH has a length of 115 to 125 amino acids. In some embodiments, the VHH has a length of 110 amino acids. In some embodiments, the VHH has a length of 111 amino acids. In some embodiments, the VHH has a length of 112 amino acids. In some embodiments, the VHH has a length of 113 amino acids. In some embodiments, the VHH has a length of 114 amino acids. In some embodiments, the VHH has a length of 115 amino acids. In some embodiments, the VHH has a length of 116 amino acids. In some embodiments, the VHH has a length of 117 amino acids. In some embodiments, the VHH has a length of 118 amino acids. In some embodiments, the VHH has a length of 119 amino acids. In some embodiments, the VHH has a length of 120 amino acids. In some embodiments, the VHH has a length of about 121 amino acids.

In some embodiments, the VHH has a length of 121 amino acids.

In some embodiments, the VHH has a length of 122 amino acids. In some embodiments, the VHH has a length of 123 amino acids. In some embodiments, the VHH has a length of 124 amino acids. In some embodiments, the VHH has a length of 125 amino acids. In some embodiments, the VHH has a length of 126 amino acids. In some embodiments, the VHH has a length of 127 amino acids. In some embodiments, the VHH has a length of 128 amino acids. In some embodiments, the VHH has a length of 129 amino acids. In some embodiments, the VHH has a length of 130 amino acids.

In some embodiments, the VHH is conjugated to the LNP via a thiol-maleimide conjugation, and the thiol-maleimide conjugation is between the DSPE-PEG 2000-maleimide and a conjugation handle that is covalently attached to the VHH.

In some embodiments, the N-terminus of the conjugation handle is covalently attached to the C-terminus of the VHH.

In some embodiments, the VHH with the attached conjugation handle has a length of 120 to 140 amino acids (e.g., 125 to 135 amino acids).

In some embodiments, the VHH with the attached conjugation handle has a length of 120-140 amino acids. In some embodiments, the VHH with the attached conjugation handle has a length of 120 amino acids. In some embodiments, the VHH with the attached conjugation handle has a length of 121 amino acids. In some embodiments, the VHH with the attached conjugation handle has a length of 122 amino acids. In some embodiments, the VHH with the attached conjugation handle has a length of 123 amino acids. In some embodiments, the VHH with the attached conjugation handle has a length of 124 amino acids. In some embodiments, the VHH with the attached conjugation handle has a length of 125 amino acids. In some embodiments, the VHH with the attached conjugation handle has a length of 126 amino acids. In some embodiments, the VHH with the attached conjugation handle has a length of 127 amino acids. In some embodiments, the VHH with the attached conjugation handle has a length of 128 amino acids.

In some embodiments, the VHH with the attached conjugation handle has a length of 125 to 135 amino acids.

In some embodiments, the VHH with the attached conjugation handle has a length of 129 amino acids.

In some embodiments, the VHH with the attached conjugation handle has a length of 130 amino acids. In some embodiments, the VHH with the attached conjugation handle has a length of 131 amino acids. In some embodiments, the VHH with the attached conjugation handle has a length of 132 amino acids. In some embodiments, the VHH with the attached conjugation handle has a length of 133 amino acids.

In some embodiments, the VHH with the attached conjugation handle has a length of about 134 amino acids.

In some embodiments, the VHH with the attached conjugation handle has a length of 135 amino acids. In some embodiments, the VHH with the attached conjugation handle has a length of 136 amino acids. In some embodiments, the VHH with the attached conjugation handle has a length of 137 amino acids. In some embodiments, the VHH with the attached conjugation handle has a length of 138 amino acids. In some embodiments, the VHH with the attached conjugation handle has a length of 139 amino acids. In some embodiments, the VHH with the attached conjugation handle has a length of 140 amino acids.

In some embodiments, the VHH comprises SEQ ID NO: 89. In some embodiments, the antibody or antigen-binding fragment is a VHH comprising CDR1, CDR2 and CDR3 sequences having at least 75%, at least 80%, at least 85%, at least 90%, or at least 95%, or 100% identity to any set of CDR1, CDR2 and CDR3 sequences in Table 10. In some embodiments, the antibody or antigen-binding fragment comprises a heavy chain variable region comprising a set of CDR1, CDR2 and CDR3 sequences that differs collectively by no more than 10, 9, 8, 7, 6, 5, 4, 3, 2, or 1 amino acids from any set of CDR1, CDR2 and CDR3 sequences disclosed in Table 10. In some embodiments, the antibody or antigen-binding fragment comprises a set of CDR1, CDR2 and CDR3 sequences that differs collectively by no more than 5 amino acids from a set of CDR1, CDR2 and CDR3 sequences disclosed in Table 10. In some embodiments, the antibody or antigen-binding fragment comprises a set of CDR1, CDR2 and CDR3 sequences that differs collectively by no more than 2 amino acids from a set of CDR1, CDR2 and CDR3 sequences disclosed in Table 10.

TABLE 10
Exemplary CDR Sequences of anti-CD5 VHH

| Anti-CD5 | | |
|---|---|---|
| VHH | CDR Sequences (Chothia) | CDR Sequences (Kabat) |
| VHH315 | CDR1: GRTFGTYT (SEQ ID NO: 61) | CDR1: TYTMG (SEQ ID NO: 234) |
| | CDR2: ISWSAGRT (SEQ ID NO: 62) | CDR2: AISWSAGRTYYADSMKG (SEQ ID |
| | CDR3: DPWTSDSDYDRLTMYDY (SEQ ID | NO: 235) |
| | NO: 63) | CDR3: DPWTSDSDYDRLTMYDY (SEQ ID |
| | | NO: 236) |
| VHH319 | CDR1: GFTFSMYS (SEQ ID NO: 64) | CDR1: MYSMS (SEQ ID NO: 237) |
| | CDR2: ISTGARDT (SEQ ID NO: 65) | CDR2: SISTGARDTAYADSVKG (SEQ ID |
| | CDR3: GDLRYGPDGYDY (SEQ ID NO: | NO: 238) |
| | 66) | CDR3: GDLRYGPDGYDY (SEQ ID NO: |
| | | 239) |
| VHH322 | CDR1: GSDFLVDA (SEQ ID NO: 67) | CDR1: VDATT (SEQ ID NO: 240) |
| | CDR2: MDIGGVT (SEQ ID NO: 68) | CDR2: IMDIGGVTEYADSVKG (SEQ ID |
| | CDR3: RGL (SEQ ID NO: 69) | NO: 241) |
| | | CDR3: RGL (SEQ ID NO: 242) |
| VHH325 | CDR1: GSDASINN (SEQ ID NO: 70) | CDR1: INNMA (SEQ ID NO: 243) |
| | CDR2: IRIGGST (SEQ ID NO: 71) | CDR2: LIRTGGSTHVADSMKG (SEQ ID |
| | CDR3: WTGSPGALSDY (SEQ ID NO: | NO: 244) |
| | 72) | CDR3: WTGSPGALSDY (SEQ ID NO: |
| | | 245) |
| VHH326 | CDR1: GRIFINYA (SEQ ID NO: 73) | CDR1: NYAAG (SEQ ID NO: 246) |
| | CDR2: ISRSGGRT (SEQ ID NO: 74) | CDR2: RISRSGGRTDYADSVKG (SEQ ID |
| | CDR3: ATVWEFTDGADQYDY (SEQ ID | NO: 247) |
| | NO: 75) | CDR3: ATVWEFTDGADQYDY (SEQ ID NO: |
| | | 248) |
| VHH327 | CDR1: GGTFSTYG (SEQ ID NO: 76) | CDR1: TYGMG (SEQ ID NO: 249) |
| | CDR2: ITGSGVGT (SEQ ID NO: 77) | CDR2: VITGSGVGTQYADSVKD (SEQ ID |
| | CDR3: GHRPGWAVIRADAYEY (SEQ ID | NO: 250) |
| | NO: 78) | CDR3: GHRPGWAVIRADAYEY (SEQ ID |
| | | NO: 251) |
| VHH329 | CDR1: GFSFSRVG (SEQ ID NO: 79) | CDR1: RVGMN (SEQ ID NO: 252) |
| | CDR2: ISSDGSRT (SEQ ID NO: 80) | CDR2: SISSDGSRTNYAHFVKG (SEQ ID |
| | CDR3: PGNS (SEQ ID NO: 81) | NO: 253) |
| | | CDR3: PGNS (SEQ ID NO: 254) |
| VHH330 | CDR1: GFPFSSSF (SEQ ID NO: 82) | CDR1: SSFMS (SEQ ID NO: 255) |
| | CDR2: IYSDGST (SEQ ID NO: 83) | CDR2: TIYSDGSTYYADSVKG (SEQ ID |
| | CDR3: VTGSI (SEQ ID NO: 84) | NO: 256) |
| | | CDR3: VTGSI (SEQ ID NO: 257) |
| VHH331 | CDR1: GRTTYN (SEQ ID NO: 85) | CDR1: TYNMG (SEQ ID NO: 258) |
| | CDR2: IDLYGRAT (SEQ ID NO: 86) | CDR2: AIDLYGRATRYANSVKG (SEQ ID |
| | CDR3: DTSLPLGVLTESQRLYGA (SEQ | NO: 259) |
| | ID NO: 87) | CDR3: DTSLPLGVLTESQRLYGA (SEQ ID |
| | | NO: 260) |

In some embodiments, the antibody or antigen-binding fragment comprises a heavy chain variable region comprising a set of CDR1, CDR2 and CDR3 sequences that differs collectively by no more than 10, 9, 8, 7, 6, 5, 4, 3, 2, or 1 amino acids from any set of CDR1, CDR2 and CDR3 sequences disclosed in Table 11. In some embodiments, the antibody or antigen-binding fragment comprises a set of CDR1, CDR2 and CDR3 sequences that differs collectively by no more than 5 amino acids from a set of CDR1, CDR2 and CDR3 sequences disclosed in Table 11. In some embodiments, the antibody or antigen-binding fragment comprises a set of CDR1, CDR2 and CDR3 sequences that differs collectively by no more than 2 amino acids from a set of CDR1, CDR2 and CDR3 sequences disclosed in Table 11.

In some embodiments, the set of CDR1, CDR2 and CDR3 sequences is a set of CDR1, CDR2 and CDR3 sequences from SEQ ID NO: 89. In some embodiments, the set of CDR1, CDR2 and CDR3 sequences is SEQ ID NO: 64, SEQ ID NO: 65 and SEQ ID NO: 66 or SEQ ID NO: 237, SEQ ID NO: 238 and SEQ ID NO: 239. In some embodiments, the set of CDR1, CDR2 and CDR3 sequences is SEQ ID NO: 64, SEQ ID NO: 65 and SEQ ID NO: 66. In some embodiments, the set of CDR1, CDR2 and CDR3 sequences is SEQ ID NO: 237, SEQ ID NO: 238 and SEQ ID NO: 239. In some embodiments, the antibody or antigen-binding fragment is a VHH.

In some embodiments, the antibody or antigen-binding fragment is a VHH comprising CDR1, CDR2 and CDR3 sequences having at least 75%, at least 80%, at least 85%, at least 90%, at least 95%, or 100% identity to any set of CDR1, CDR2 and CDR3 sequences in Table 11. In some embodiments, the antibody or antigen-binding fragment is a VHH having at least 75%, at least 80%, at least 85%, at least 90%, at least 95%, or 100% identity to any one of SEQ ID Nos: 88-116.

In some embodiments, the targeting moiety comprises an anti-CD5 antibody or antigen-binding fragment thereof, and one or more of (i) an anti-CD2 antibody or antigen-binding fragment thereof, (ii) an anti-CD3 antibody or antigen-binding fragment thereof, (iii) an anti-CD7 antibody or antigen-binding fragment thereof, and (iv) an anti-CD8 antibody or antigen-binding fragment thereof.

In some embodiments, the targeting moiety comprises an anti-CD5 antibody or antigen-binding fragment thereof and further comprises an anti-CD2 antibody or antigen-binding fragment thereof, an anti-CD3 antibody or antigen-binding fragment thereof, an anti-CD7 antibody or antigen-binding fragment thereof, or an anti-CD8 antibody or antigen-binding fragment thereof.

TABLE 11
Exemplary anti-CD5, anti-CD7, anti-CD8 VHH Sequences

| Description | Sequence |
|---|---|
| anti-CD5 VHH315 | QVQLVESGGGLVQAGGSLRLSCAASGRTFGTYTMGWFRQAPGREREFVAAISWSAGRTYY |
| | ADSMKGRFTISRDNAKNTVYLQMNSLKPEDTAVYYCAADPWTSDSDYDRLTMYDYWGQGT |
| | QVTVSS |
| | (SEQ ID NO: 88) |
| anti-CD5 VHH319 | QVQLVESGGGLVQPGGSLRLSCAASGFTFSMYSMSWVRQAPGKRLEWVSSISTGARDTAY |
| | ADSVKGRFTISRDNADNTLYLHMNNLKPEDTAVYYCANGDLRYGPDGYDYRGQGTQVTVS |
| | S |
| | (SEQ ID NO: 89) |
| anti-CD5 VHH322 | QLQLVESGGGLVQPGGSLRLSCAASGSDFLVDATTWFRQAPGNQREFVAIMDIGGVTEYA |
| | DSVKGRFTISRDHTKNTVYLQMNSLKVEDTAVYYCNTRGLWGQGTLVTVSS |
| | (SEQ ID NO: 90) |
| anti-CD5 VHH325 | QVQLVESGGGLVQPGGSLRLSCAASGSDASTNNMAWYRQAPGKEREFVALIRTGGSTHVA |
| | DSMKGRFTISRENAKNTVYLQMNSLKPEDTAVYYCVIWTGSPGALSDYWGQGTQVTVSS |
| | (SEQ ID NO: 91) |
| anti-CD5 VHH326 | QVQLVESGGGLVQPGGSLRLSCAASGRTFINYAAGWFRQAPGKEREFVARISRSGGRTDY |
| | ADSVKGRFTISRDNAKSTVYLQMNSLRPEDTAVYYCAEATVWEFTDGADQYDYWGQGTQV |
| | TVSS |
| | (SEQ ID NO: 92) |
| anti-CD5 | QVQLVESGGGLVQPGGSLRLSCVASGGTESTYGMGWFRQAPGKEREFVAVITGSGVGTQY |
| VHH327 | ADSVKDRFTISRENAKNTVYLQMNTLKLEDTAVYYCVSGHRPGWAVIRADAYEYWGQGTQ |
| | VTVSS |
| | (SEQ ID NO: 93) |
| anti-CD5 | QLQLVESGGGLVQPGESLRLSCAASGFSFSRVGMNWYRQAPGKERELVASISSDGSRTNY |
| VHH329 | AHFVKGRFTISRDNVKNMVYLQMNSLKLEDTAVYYCNVPGNSWGQGTQVTVSS |
| | (SEQ ID NO: 94) |
| anti-CD5 | EVQLVESGGGLVQPGGSLRLSCAASGFPFSSSFMSWVRQAPGKGLEWVSTIYSDGSTYYA |
| VHH330 | DSVKGRFTISRDNAKKTAYLQMNSLKAEDTAVYYCATVTGSIRGQGTQVTVSS |
| | (SEQ ID NO: 95) |
| anti-CD5 | QVQLVESGGGLVQAGASLRLSCAASGRTTYNMGWFRHAPGKDREFVAAIDLYGRATRYAN |
| VHH331 | SVKGRFTISRDNAKNTVYLQMNSLKPEDTAVYYCAADTSLPLGVLTESQRLYGAWGQGTQ |
| | VTVSS |
| | (SEQ ID NO: 96) |
| anti-CD8 | EVQLVESGGGLVQAGGSLRLSCAASGFTEDDYAIGWFRQAPGKGREGVLCIRIFDRHTYS |
| VHH5v2 | ADSVKGRFTISSDNAQNTVYLHMNSLKPEDTAVYYCAAGSFWGCTRPEGDMDYWGKGTQV |
| | TVSS |
| | (SEQ ID NO: 97) |
| anti-CD8 | EVQLVESGGGLVQPGGSLRLSCAASGFTEDDYAIGWFRQAPGKGREGVACIRIFDRHTYY |
| VHHhu2C8v144 | ADSVKGRFTISSDNSKNTVYLQMNSLRAEDTATYYCAAGSFWGCTRPEGDMDYFGQGTLV |
| | QVQSA |
| | (SEQ ID NO: 98) |
| anti-CD8 | QVQLVESGGGLVQAGGSLRLSCAASGRTFSGYVMGWFRQAPGKQRKFVAAISRGGLSTSY |
| VHH S97 | ADSVKGRFTISRDNAKNTVFLQMNTLKPEDTAVYYCAADRSDLYEITAASNIDSWGQGTL |
| | VTVSS |
| | (SEQ ID NO: 99) |
| anti-CD7 | MDVQLQESGGGLVQAGGSLRLSCAVSGYPYSSYCMGWFRQAPGKEREGVAAIDSDGRTRY |
| litVHHpb6 | ADSVKGRFTISQDNAKNTLYLQMNRMKPEDTAMYYCAARFGPMGCVDLSTLSFGHWGQGT |
| | QVTVSIT |
| | (SEQ ID NO: 100) |
| anti-CD5-CD5 | QVQLVESGGGLVQPGGSLRLSCAASGFTFSMYSMSWVRQAPGKRLEWVSSISTGARDTAY |
| | ADSVKGRFTISRDNADNTLYLHMNNLKPEDTAVYYCANGDLRYGPDGYDYRGQGTQVTVS |
| | SGGGGSGGGGSGGGGSGGGGSQVQLVESGGGLVQPGGSLRLSCAASGFTFSMYSMSWVRQ |
| | APGKRLEWVSSISTGARDTAYADSVKGRFTISRDNADNTLYLHMNNLKPEDTAVYYCANG |
| | DLRYGPDGYDYRGQGTQVTVSS |
| | (SEQ ID NO: 101) |
| anti-CD7-CD7 | MDVQLQESGGGLVQAGGSLRLSCAVSGYPYSSYCMGWFRQAPGKEREGVAAIDSDGRTRY |
| | ADSVKGRFTISQDNAKNTLYLQMNRMKPEDTAMYYCAARFGPMGCVDLSTLSFGHWGQGT |
| | QVTVSITGGGGSGGGGSGGGGSGGGGSMDVQLQESGGGLVQAGGSLRLSCAVSGYPYSSY |
| | CMGWFRQAPGKEREGVAAIDSDGRTRYADSVKGRFTISQDNAKNTLYLQMNRMKPEDTAM |
| | YYCAARFGPMGCVDLSTLSFGHWGQGTQVTVSIT |
| | (SEQ ID NO: 102) |
| anti-CD8-CD8 | EVQLVESGGGLVQPGGSLRLSCAASGFTFDDYAIGWFRQAPGKGREGVACIRIFDRHTYY |
| | ADSVKGRFTISSDNSKNTVYLQMNSLRAEDTATYYCAAGSFWGCTRPEGDMDYFGQGTLV |
| | QVQSAGGGGSGGGGSGGGGSGGGGSEVQLVESGGGLVQPGGSLRLSCAASGFTEDDYAIG |
| | WFRQAPGKGREGVACIRIFDRHTYYADSVKGRFTISSDNSKNTVYLQMNSLRAEDTATYY |
| | CAAGSFWGCTRPEGDMDYFGQGTLVQVQSA |
| | (SEQ ID NO: 103) |
| anti-CD5-CD7 | QVQLVESGGGLVQPGGSLRLSCAASGFTFSMYSMSWVRQAPGKRLEWVSSISTGARDTAY |
| | ADSVKGRFTISRDNADNTLYLHMNNLKPEDTAVYYCANGDLRYGPDGYDYRGQGTQVTVS |
| | SGGGGSGGGGSGGGGSGGGGSMDVQLQESGGGLVQAGGSLRLSCAVSGYPYSSYCMGWER |
| | QAPGKEREGVAAIDSDGRTRYADSVKGRFTISQDNAKNTLYLQMNRMKPEDTAMYYCAAR |
| | FGPMGCVDLSTLSFGHWGQGTQVTVSIT |
| | (SEQ ID NO: 104) |
| anti-CD7-CD5 | MDVQLQESGGGLVQAGGSLRLSCAVSGYPYSSYCMGWFRQAPGKEREGVAAIDSDGRTRY |
| | ADSVKGRFTISQDNAKNTLYLQMNRMKPEDTAMYYCAARFGPMGCVDLSTLSFGHWGQGT |
| | QVTVSITGGGGGGGGSGGGGSGGGGSQVQLVESGGGLVQPGGSLRLSCAASGFTFSMYS |
| | MSWVRQAPGKRLEWVSSISTGARDTAYADSVKGRFTISRDNADNTLYLHMNNLKPEDTAV |
| | YYCANGDLRYGPDGYDYRGQGTQVTVSS |
| | (SEQ ID NO: 105) |
| anti-CD5-CD8 | QVQLVESGGGLVQPGGSLRLSCAASGFTFSMYSMSWVRQAPGKRLEWVSSISTGARDTAY |
| | ADSVKGRFTISRDNADNTLYLHMNNLKPEDTAVYYCANGDLRYGPDGYDYRGQGTQVTVS |
| | SGGGGSGGGGSGGGGSGGGGSEVQLVESGGGLVQPGGSLRLSCAASGFTEDDYAIGWFRQ |
| | APGKGREGVACIRIFDRHTYYADSVKGRFTISSDNSKNTVYLQMNSLRAEDTATYYCAAG |
| | SFWGCTRPEGDMDYFGQGTLVQVQSA |
| | (SEQ ID NO: 106) |
| anti-CD8-CD5 | EVQLVESGGGLVQPGGSLRLSCAASGFTEDDYAIGWFRQAPGKGREGVACIRIFDRHTYY |
| | ADSVKGRFTISSDNSKNTVYLQMNSLRAEDTATYYCAAGSFWGCTRPEGDMDYFGQGTLV |
| | QVQSAGGGGSGGGGSGGGGSGGGGSQVQLVESGGGLVQPGGSLRLSCAASGFTFSMYSMS |
| | WVRQAPGKRLEWVSSISTGARDTAYADSVKGRFTISRDNADNTLYLHMNNLKPEDTAVYY |
| | CANGDLRYGPDGYDYRGQGTQVTVSS |
| | (SEQ ID NO: 107) |
| anti-CD8-CD7 | EVQLVESGGGLVQPGGSLRLSCAASGFTFDDYAIGWFRQAPGKGREGVACIRIFDRHTYY |
| | ADSVKGRFTISSDNSKNTVYLQMNSLRAEDTATYYCAAGSFWGCTRPEGDMDYFGQGTLV |
| | QVQSAGGGGSGGGGSGGGGSGGGGSMDVQLQESGGGLVQAGGSLRLSCAVSGYPYSSYCM |
| | GWFRQAPGKEREGVAAIDSDGRTRYADSVKGRFTISQDNAKNTLYLQMNRMKPEDTAMYY |
| | CAARFGPMGCVDLSTLSFGHWGQGTQVTVSIT |
| | (SEQ ID NO: 108) |
| anti-CD7-CD8 | MDVQLQESGGGLVQAGGSLRLSCAVSGYPYSSYCMGWFRQAPGKEREGVAAIDSDGRTRY |
| | ADSVKGRFTISQDNAKNTLYLQMNRMKPEDTAMYYCAARFGPMGCVDLSTLSFGHWGQGT |
| | QVTVSITGGGGSGGGGSGGGGSGGGGSEVQLVESGGGLVQPGGSLRLSCAASGFTEDDYA |
| | IGWFRQAPGKGREGVACIRIFDRHTYYADSVKGRFTISSDNSKNTVYLQMNSLRAEDTAT |
| | YYCAAGSFWGCTRPEGDMDYFGQGTLVQVQSA |
| | (SEQ ID NO: 109) |
| anti-CD5-CD5- | QVQLVESGGGLVQPGGSLRLSCAASGFTFSMYSMSWVRQAPGKRLEWVSSISTGARDTAY |
| CD5 | ADSVKGRFTISRDNADNTLYLHMNNLKPEDTAVYYCANGDLRYGPDGYDYRGQGTQVTVS |
| | SGGGGSGGGGSGGGGSGGGGSQVQLVESGGGLVQPGGSLRLSCAASGFTFSMYSMSWVRQ |
| | APGKRLEWVSSISTGARDTAYADSVKGRFTISRDNADNTLYLHMNNLKPEDTAVYYCANG |
| | DLRYGPDGYDYRGQGTQVTVSSGGGGSGGGGSGGGGSGGGGSQVQLVESGGGLVQPGGSL |
| | RLSCAASGFTFSMYSMSWVRQAPGKRLEWVSSISTGARDTAYADSVKGRFTISRDNADNT |
| | LYLHMNNLKPEDTAVYYCANGDLRYGPDGYDYRGQGTQVTVSS |
| | (SEQ ID NO: 110) |
| anti-CD5-CD7- | QVQLVESGGGLVQPGGSLRLSCAASGFTFSMYSMSWVRQAPGKRLEWVSSISTGARDTAY |
| CD8 | ADSVKGRFTISRDNADNTLYLHMNNLKPEDTAVYYCANGDLRYGPDGYDYRGQGTQVTVS |
| | SGGGGSGGGGSGGGGSGGGGSMDVQLQESGGGLVQAGGSLRLSCAVSGYPYSSYCMGWFR |
| | QAPGKEREGVAAIDSDGRTRYADSVKGRFTISQDNAKNTLYLQMNRMKPEDTAMYYCAAR |
| | FGPMGCVDLSTLSFGHWGQGTQVTVSITGGGGSGGGGSGGGGSGGGGSEVQLVESGGGLV |
| | QPGGSLRLSCAASGFTEDDYAIGWFRQAPGKGREGVACIRIFDRHTYYADSVKGRFTISS |
| | DNSKNTVYLQMNSLRAEDTATYYCAAGSFWGCTRPEGDMDYFGQGTLVQVQSA |
| | (SEQ ID NO: 111) |
| anti-CD5-CD8- | QVQLVESGGGLVQPGGSLRLSCAASGFTFSMYSMSWVRQAPGKRLEWVSSISTGARDTAY |
| CD7 | ADSVKGRFTISRDNADNTLYLHMNNLKPEDTAVYYCANGDLRYGPDGYDYRGQGTQVTVS |
| | SGGGGSGGGGSGGGGSGGGGSEVQLVESGGGLVQPGGSLRLSCAASGFTEDDYAIGWFRQ |
| | APGKGREGVACIRIFDRHTYYADSVKGRFTISSDNSKNTVYLQMNSLRAEDTATYYCAAG |
| | SFWGCTRPEGDMDYFGQGTLVQVQSAGGGGSGGGGSGGGGSGGGGSMDVQLQESGGGLVQ |
| | AGGSLRLSCAVSGYPYSSYCMGWFRQAPGKEREGVAAIDSDGRTRYADSVKGRFTISQDN |
| | AKNTLYLQMNRMKPEDTAMYYCAARFGPMGCVDLSTLSFGHWGQGTQVTVSIT |
| | (SEQ ID NO: 112) |
| anti-CD7-CD5- | MDVQLQESGGGLVQAGGSLRLSCAVSGYPYSSYCMGWFRQAPGKEREGVAAIDSDGRTRY |
| CD8 | ADSVKGRFTISQDNAKNTLYLQMNRMKPEDTAMYYCAARFGPMGCVDLSTLSFGHWGQGT |
| | QVTVSITGGGGSGGGGSGGGGSGGGGSQVQLVESGGGLVQPGGSLRLSCAASGFTFSMYS |
| | MSWVRQAPGKRLEWVSSISTGARDTAYADSVKGRFTISRDNADNTLYLHMNNLKPEDTAV |
| | YYCANGDLRYGPDGYDYRGQGTQVTVSSGGGGSGGGGSGGGGSGGGGSEVQLVESGGGLV |
| | QPGGSLRLSCAASGFTEDDYAIGWFRQAPGKGREGVACIRIFDRHTYYADSVKGRFTISS |
| | DNSKNTVYLQMNSLRAEDTATYYCAAGSFWGCTRPEGDMDYFGQGTLVQVQSA |
| | (SEQ ID NO: 113) |
| anti-CD7-CD8- | MDVQLQESGGGLVQAGGSLRLSCAVSGYPYSSYCMGWFRQAPGKEREGVAAIDSDGRTRY |
| CD5 | ADSVKGRFTISQDNAKNTLYLQMNRMKPEDTAMYYCAARFGPMGCVDLSTLSFGHWGQGT |
| | QVTVSITGGGGSGGGGSGGGGSGGGGSEVQLVESGGGLVQPGGSLRLSCAASGFTEDDYA |
| | IGWFRQAPGKGREGVACIRIFDRHTYYADSVKGRFTISSDNSKNTVYLQMNSLRAEDTAT |
| | YYCAAGSFWGCTRPEGDMDYFGQGTLVQVQSAGGGGSGGGGSGGGGSGGGGSQVQLVESG |
| | GGLVQPGGSLRLSCAASGFTFSMYSMSWVRQAPGKRLEWVSSISTGARDTAYADSVKGRF |
| | TISRDNADNTLYLHMNNLKPEDTAVYYCANGDLRYGPDGYDYRGQGTQVTVSS |
| | (SEQ ID NO: 114) |
| anti-CD8-CD5- | EVQLVESGGGLVQPGGSLRLSCAASGFTEDDYAIGWFRQAPGKGREGVACIRIFDRHTYY |
| CD7 | ADSVKGRFTISSDNSKNTVYLQMNSLRAEDTATYYCAAGSFWGCTRPEGDMDYFGQGTLV |
| | QVQSAGGGGSGGGGSGGGGSGGGGSQVQLVESGGGLVQPGGSLRLSCAASGFTFSMYSMS |
| | WVRQAPGKRLEWVSSISTGARDTAYADSVKGRFTISRDNADNTLYLHMNNLKPEDTAVYY |
| | CANGDLRYGPDGYDYRGQGTQVTVSSGGGGSGGGGSGGGGSGGGGSMDVQLQESGGGLVQ |
| | AGGSLRLSCAVSGYPYSSYCMGWFRQAPGKEREGVAAIDSDGRTRYADSVKGRFTISQDN |
| | AKNTLYLQMNRMKPEDTAMYYCAARFGPMGCVDLSTLSFGHWGQGTQVTVSIT |
| | (SEQ ID NO: 115) |
| anti-CD8-CD7- | EVQLVESGGGLVQPGGSLRLSCAASGFTFDDYAIGWFRQAPGKGREGVACIRIFDRHTYY |
| CD5 | ADSVKGRFTISSDNSKNTVYLQMNSLRAEDTATYYCAAGSFWGCTRPEGDMDYFGQGTLV |
| | QVQSAGGGGSGGGGSGGGGSGGGGSMDVQLQESGGGLVQAGGSLRLSCAVSGYPYSSYCM |
| | GWFRQAPGKEREGVAAIDSDGRTRYADSVKGRFTISQDNAKNTLYLQMNRMKPEDTAMYY |
| | CAARFGPMGCVDLSTLSFGHWGQGTQVTVSITGGGGSGGGGSGGGGSGGGGSQVQLVESG |
| | GGLVQPGGSLRLSCAASGFTFSMYSMSWVRQAPGKRLEWVSSISTGARDTAYADSVKGRF |
| | TISRDNADNTLYLHMNNLKPEDTAVYYCANGDLRYGPDGYDYRGQGTQVTVSS |
| | (SEQ ID NO: 116) |

### Conjugation of Targeting Moiety to Lipid Nanoparticle

In some embodiments, a targeting moiety is conjugated to a delivery vehicle via a conjugation handle. As used herein, the term “conjugation handle” refers to a short peptide containing one or more amino acids (e.g., 1-20 amino acids). In some embodiments, the conjugation handle includes at least one reactive group (e.g., azide, tetrazine, or thiol) allowing conjugation between two moieties via, e.g., chemical, enzymatic, or enzyme-catalyzed reaction. The reactive group in the conjugation handle may be a functional group of an amino acid on the “conjugation handle” or attached anywhere on the “conjugation handle” (e.g., at the N-terminus, C-terminus, or internal to the conjugation handle) directly or indirectly. In some embodiments, the reactive group described herein is suitable for a “click chemistry” reaction. In some embodiments, the conjugation handle comprises one or more glycine, alanine, cysteine, serine and/or glutamine. A “conjugation handle” may contain, for example, 5-10 amino acids, such as 5, 6, 7, 8, 9, or 10 amino acids. A “conjugation handle” may be attached to (e.g., covalently attached to) a moiety to be conjugated, such as a targeting moiety. In such cases, a conjugation handle may be a naturally-occurring or modified amino acid in the moiety (e.g., targeting moiety) containing a reactive group. In other embodiments, a conjugation handle may be attached to a moiety, directly or indirectly. As used herein, in some embodiments, “conjugation handle” also refers to a short peptide that is part of the conjugation product formed by joining two moieties.

For example, and without limitation, a conjugation handle links, joins or conjugates a targeting moiety and a delivery vehicle. In some embodiments, an anti-CD2, an anti-CD3, an anti-CD5, an anti-CD7, or an anti-CD8 antibody or antigen-binding fragment thereof is conjugated to a lipid nanoparticle via a conjugation handle. In some embodiments, the anti-CD2 antibody is attached (e.g., covalently attached) to a conjugation handle. In some embodiments, the anti-CD3 antibody is attached (e.g., covalently attached) to a conjugation handle. In some embodiments, the anti-CD5 antibody is attached (e.g., covalently attached) to a conjugation handle. In some embodiments, the anti-CD7 antibody is attached (e.g., covalently attached) to a conjugation handle. In some embodiments, the anti-CD8 antibody is attached (e.g., covalently attached) to a conjugation handle.

In some embodiments, after a chemical or enzymatic conjugation reaction, the conjugation handle that links the targeting moiety and delivery vehicle is retained after the reaction in the targeted delivery vehicle. In other words, in some embodiments, the conjugation handle is incorporated in the conjugation product. In some embodiments, a VHH is attached (e.g., covalently attached) to a conjugation handle. In some embodiments, an anti-CD2 VHH is attached (e.g., covalently attached) to a conjugation handle. In some embodiments, an anti-CD3 VHH is attached (e.g., covalently attached) to a conjugation handle. In some embodiments, an anti-CD5 VHH is attached (e.g., covalently attached) to a conjugation handle. In some embodiments, an anti-CD7 VHH is attached (e.g., covalently attached) to a conjugation handle. In some embodiments, an anti-CD8 VHH is attached (e.g., covalently attached) to a conjugation handle.

In some embodiments, a VHH antibody is conjugated to a lipid nanoparticle (LNP). In some embodiments, the VHH antibody is conjugated to a PEG molecule in the LNP. In some embodiments, the VHH antibody is conjugated to a modified-PEG molecule in the LNP. In some embodiments, the conjugation is covalent.

In some embodiments, the targeting moiety is conjugated to the lipid nanoparticle via one of many bioconjugation or conjugation chemistries. “Bioconjugation chemistry” or “conjugation chemistry,” as used herein, refers any method capable of forming a covalent bond between a biomolecule (e.g., a conjugation handle, which may be attached to a targeting moiety, such as, without limitation, a VHH of the present disclosure) and a second molecule (e.g., a delivery vehicle, such as without limitation, a lipid nanoparticle, more specifically, a PEG or modified-PEG on a LNP). In some embodiments, bioconjugation chemistry results in a covalent bond that is stable. In some embodiments, bioconjugation chemistry results in a covalent bond that is irreversible. In some embodiments, bioconjugation chemistry results in a covalent bond that is reversible.

In some embodiments, the targeting moiety is conjugated to the nanoparticle via a chemical reaction. In some embodiments, the targeting moiety is conjugated to the lipid nanoparticle via a chemical reaction. In some embodiments, the targeting moiety is conjugated to a viral particle via a chemical reaction. In some embodiments, the targeting moiety is conjugated to an engineered viral particle via a chemical reaction. In some embodiments, the chemical reaction is a “click chemistry” reaction. “Click chemistry” refers to a chemical attachment approach introduced by K. Barry Sharpless of The Scripps Research Institute, describing chemistry tailored to generate covalent bonds quickly and reliably by joining small units comprising reactive groups together. Click chemistry does not refer to a specific reaction, but to a concept including reactions that mimic reactions found in nature. Click chemistry reactions and suitable chemical groups for click chemistry reactions (e.g., conjugation handles) are well known to those of skill in the art, and include, for example and without limitation, terminal alkynes, azides, strained alkynes, dienes, dienophiles, alkoxyamines, carbonyls, phosphines, hydrazides, thiols, tetrazines, and alkenes. In some embodiments, the chemical reactions are biorthogonal, proceeding quickly and selectively without side reactions with endogenous functional groups.

In some embodiments, the chemical reaction is a thiol-maleimide conjugation. In some embodiments, the chemical reaction is a transcyclooctene (TCO)-tetrazine conjugation. In some embodiments, the chemical reaction is an azide-alkyne cycloaddition (AAC) conjugation. In some embodiments, the chemical reaction is an azide-dibenzocyclooctyne (DBCO) conjugation.

In some embodiments, a conjugation handle attached to a targeting moiety is conjugated to a lipid nanoparticle via a thiol-maleimide conjugation. In some embodiments, the targeting moiety is conjugated to a lipid nanoparticle via a thiol-maleimide conjugation. In some embodiments, a conjugation handle, which is attached to (e.g., covalently attached to) the targeting moiety (e.g., attached at the C-terminus of the targeting moiety, e.g., at the C-terminus of a VHH targeting moiety) is conjugated to the lipid nanoparticle via a thiol-maleimide conjugation. In some embodiments, the targeting moiety is conjugated to a viral particle via a thiol-maleimide conjugation. In some embodiments, the targeting moiety is conjugated to an engineered viral particle via a thiol-maleimide conjugation.

In some embodiments, the anti-CD5 antibody or antigen-binding fragment is conjugated to the conjugating PEG-lipid in the delivery vehicle via a thiol-maleimide conjugation.

In some embodiments, the anti-CD5 antibody or antigen-binding fragment is conjugated to the conjugating PEG-lipid in the delivery vehicle via a thiol-maleimide conjugation between the conjugating PEG-lipid and the conjugation handle.

In some embodiments, the targeting moiety is conjugated via an enzymatic reaction. In some embodiments, the targeting moiety is conjugated to the nanoparticle via an enzymatic reaction. In some embodiments, the targeting moiety is conjugated to the lipid nanoparticle via an enzymatic reaction. In some embodiments, the targeting moiety is conjugated to the viral particle via an enzymatic reaction. In some embodiments, the targeting moiety is conjugated to the engineered viral particle via an enzymatic reaction. In some embodiments, the enzymatic reaction comprises a conjugation-catalyzing enzyme that reacts at a specific C-terminal amino acid (e.g., glutamine) or amino acid sequence (e.g., LPXT/Gn motif, wherein n=1-5 and X is any amino acid) on the conjugation handle. In some embodiments, the enzymatic reaction comprises a conjugation-catalyzing enzyme that reacts at an internal amino acid (e.g., glutamine) on the targeting moiety. In some embodiments, the enzymatic reaction is transglutaminase-catalyzed glutamine-amine conjugation. In some embodiments, the enzymatic reaction is sortase-catalyzed conjugation. In some embodiments, the enzymatic reaction is formylglycine-generating enzyme (FGE)-catalyzed conjugation. In some embodiments, the enzymatic reaction is glycosyltransferase-catalyzed conjugation.

In some embodiments, the anti-CD5 antibody or antigen-binding fragment is conjugated to the nanoparticle. In some embodiments, the anti-CD5 antibody or antigen-binding fragment is conjugated to the lipid nanoparticle. In some embodiments, the anti-CD5 antibody or antigen-binding fragment is conjugated to the viral particle. In some embodiments, the anti-CD5 antibody or antigen-binding fragment is conjugated to the engineered viral particle.

In some embodiments, the anti-CD5 antibody or antigen-binding fragment is conjugated to a conjugating PEG-lipid in the delivery vehicle.

In some embodiments, the anti-CD5 antibody or antigen-binding fragment is covalently attached to a conjugation handle.

In some embodiments, the conjugation handle is covalently attached to the C-terminus of the heavy chain of the anti-CD5 antibody or antigen-binding fragment.

In some embodiments, the conjugation is a thiol-maleimide conjugation. In some embodiments, the conjugation is an azide-dibenzocyclooctyne (DBCO) conjugation. In some embodiments, the conjugation is a transcyclooctene (TCO)-tetrazine conjugation. In some embodiments, the conjugation is a transglutaminase-catalyzed glutamine-amine conjugation. In some embodiments, the conjugation is a sortase-catalyzed conjugation. In some embodiments, the conjugation is a sortase-catalyzed LPXT/(G)n-(G)n conjugation, where X is any amino acid and n=1-5.

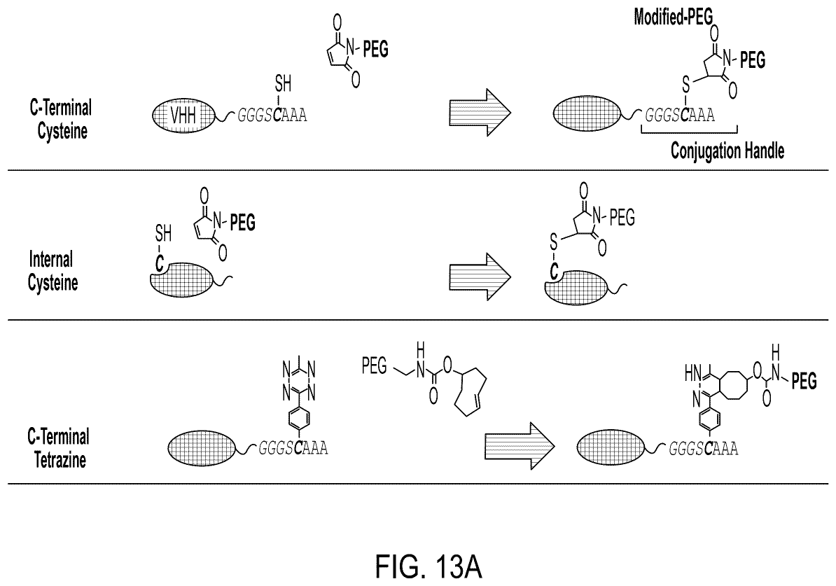

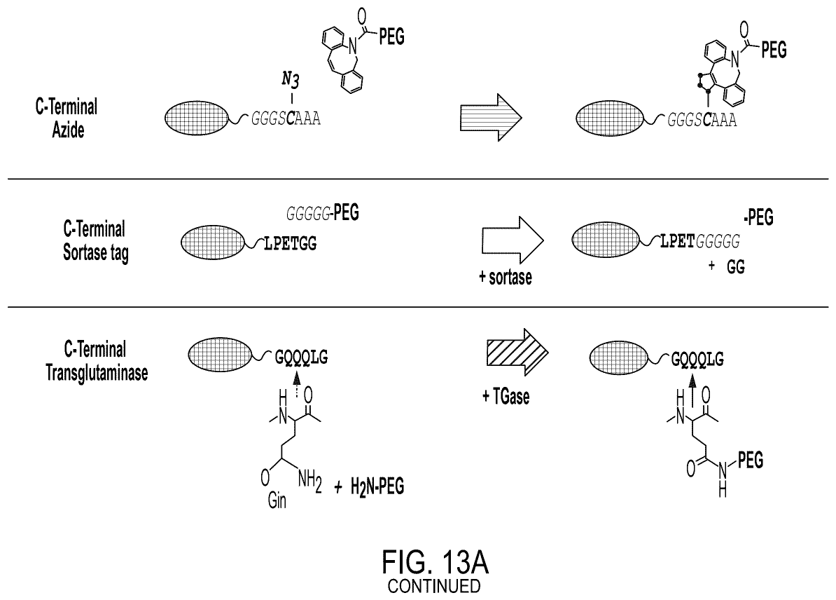

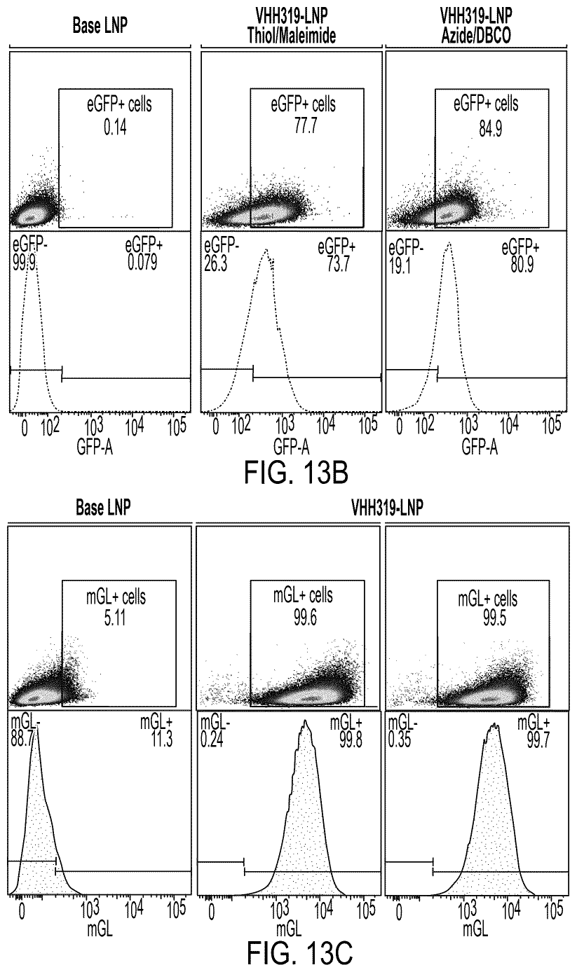

In some embodiments, the anti-CD5 antibody or antigen-binding fragment is conjugated to the nanoparticle via a thiol-maleimide conjugation, an azide-dibenzocyclooctyne (DBCO) conjugation, a transcyclooctene (TCO)-tetrazine conjugation, a transglutaminase-catalyzed glutamine-amine conjugation, or a sortase-catalyzed conjugation. In some embodiments, the anti-CD5 antibody or antigen-binding fragment is conjugated to the nanoparticle via a thiol-maleimide conjugation. In some embodiments, the anti-CD5 antibody or antigen-binding fragment is conjugated to the nanoparticle via an azide-dibenzocyclooctyne (DBCO) conjugation. In some embodiments, the anti-CD5 antibody or antigen-binding fragment is conjugated to the nanoparticle via a transcyclooctene (TCO)-tetrazine conjugation. In some embodiments, the anti-CD5 antibody or antigen-binding fragment is conjugated to the nanoparticle via a transglutaminase-catalyzed glutamine-amine conjugation. In some embodiments, the anti-CD5 antibody or antigen-binding fragment is conjugated to the nanoparticle via a sortase-catalyzed conjugation. In some embodiments, exemplary conjugation handles are selected from Table 12A. In some embodiments, exemplary conjugation handles are selected from any one of SEQ ID NOs: 117-121. In some embodiments, a conjugation chemistry is selected from FIG. 13A.

In some embodiments, the anti-CD5 antibody VHH is conjugated to the LNP via a thiol-maleimide, an azide-dibenzocyclooctyne (DBCO), a transcyclooctene (TCO)-tetrazine, a transglutaminase-catalyzed glutamine-amine conjugation or a sortase-catalyzed conjugation.

In some embodiments, the anti-CD5 antibody VHH is conjugated to the LNP via a thiol-maleimide conjugation. In some embodiments, the anti-CD5 antibody VHH is conjugated to the LNP via an azide-dibenzocyclooctyne (DBCO). In some embodiments, the anti-CD5 antibody VHH is conjugated to the LNP via a transcyclooctene (TCO)-tetrazine. In some embodiments, the anti-CD5 antibody VHH is conjugated to the LNP via a transglutaminase-catalyzed glutamine-amine conjugation. In some embodiments, the anti-CD5 antibody VHH is conjugated to the LNP via a sortase-catalyzed conjugation.

In some embodiments, the delivery vehicle is an LNP comprising two PEG-lipids, one of which is the conjugating PEG-lipid. In some embodiments, the LNP comprises two PEG-lipids, one of which is a conjugating PEG-lipid, and the thiol-maleimide conjugation is between the conjugating PEG-lipid and the conjugation handle.

In some embodiments, the conjugating PEG-lipid comprises a long chain fatty acid.

In some embodiments, the conjugating PEG-lipid is DSPE-PEG 2000-maleimide.

In some embodiments, the conjugation handle is 5-10 amino acids in length. In some embodiments, the conjugation handle is 5 amino acids in length. In some embodiments, the conjugation handle is 6 amino acids in length. In some embodiments, the conjugation handle is 7 amino acids in length. In some embodiments, the conjugation handle is 8 amino acids in length. In some embodiments, the conjugation handle is 9 amino acids in length. In some embodiments, the conjugation handle is 10 amino acids in length.

In some embodiments, the anti-CD5 antibody or antigen-binding fragment is conjugated via a thiol-maleimide conjugation. In some embodiments, the conjugation handle comprises SEQ ID NO: 120, or SEQ ID NO: 344. In some embodiments, the conjugation handle comprises SEQ ID NO: 120. In some embodiments, the conjugation handle comprises SEQ ID NO: 344.

In some embodiments, the conjugation handle comprises SEQ ID NO: 120.

In some embodiments, the conjugation handle consists of SEQ ID NO: 120.

In some embodiments, the anti-CD5 antibody or antigen-binding fragment is conjugated via the conjugation handle of SEQ ID NO: 120. In some embodiments, the anti-CD5 antibody or antigen-binding fragment is conjugated via a thiol-maleimide conjugation using the cysteine in the conjugation handle of SEQ ID NO: 120.

TABLE 12A
Exemplary Conjugation Handles

| Conjugation Handle | Sequence |
|---|---|
| Sortase Conjugation | LPET (SEQ ID NO: 117)¹ |
| Handle | |
| Q3 Conjugation | GQQQLG (SEQ ID NO: 118) |
| Handle | |
| “G3C” | GGGC (SEQ ID NO: 119) |
| “G3S-Cys-AAA” | GGGSCAAA (SEQ ID NO: 120) |
| “G3S-Q3-LG” | GGGSGQQQLG (SEQ ID NO: 121) |
| “G4S-Cys-AAA” | GGGGSCAAA (SEQ ID NO: 343) |
| “G4SG4S” | GGGGSGGGGS (SEQ ID NO: 344) |
| “G4SG4S-Cys-AS” | GGGGSGGGGSCAS (SEQ ID NO: 345) |

¹Sortase cleaves between T and G in LPXT/(Glycine)n-(Glycine)n wherein, e.g., n = 1-5 and X is any amino acid, and LPXT (for example, LPET) is incorporated in the final product.

In some embodiments, the anti-CD5 VHH sequence comprising a conjugation handle is selected from any one of SEQ ID NO: 122-124, as shown in Table 12B. In some embodiments, the anti-CD5 VHH sequence with conjugation handle is at least 75%, at least 80%, at least 85%, at least 90%, or at least 95% identical to any one of SEQ ID NO: 122-124. In some embodiments, the anti-CD5 VHH sequence with a conjugation handle is identical to any one of SEQ ID NO: 122-124.

In some embodiments, the anti-CD5 antibody or antigen-binding fragment is covalently attached to the conjugation handle, and the sequence of the anti-CD5 antibody or antigen-binding fragment with attached conjugation handle comprises SEQ ID NO: 122. In some embodiments, the anti-CD5 antibody or antigen-binding fragment is covalently attached to the conjugation handle, and the sequence of the anti-CD5 antibody or antigen-binding fragment with attached conjugation handle comprises SEQ ID NO: 123. In some embodiments, the anti-CD5 antibody or antigen-binding fragment is covalently attached to the conjugation handle, and the sequence of the anti-CD5 antibody or antigen-binding fragment with attached conjugation handle comprises SEQ ID NO: 124.

In some embodiments, the VHH is conjugated to the LNP via a thiol-maleimide conjugation, and the thiol-maleimide conjugation is between the DSPE-PEG 2000-maleimide and a conjugation handle that is covalently attached to the VHH.

In some embodiments, the N-terminus of the conjugation handle is covalently attached to the C-terminus of the VHH.

In some embodiments, the sequence of the VHH with the attached conjugation handle comprises SEQ ID NO: 122.

In some embodiments, the sequence of the VHH with the attached conjugation handle is SEQ ID NO: 122.

TABLE 12B
Exemplary anti-CD5 VHH sequences with
conjugation handles

Anti-CD5 VHH319 with C-terminal G3S-Cys-AAA
(Conjugation handle G3S-Cys-CAAA is underlined;
conjugation to delivery vehicle is through C in
bold italics; WT N82 is in bold italics)
QVQLVESGGGLVQPGGSLRLSCAASGFTFSMYSMSWVRQAPGKRLE
WVSSISTGARDTAYADSVKGRFTISRDNADNTLYLHMNNLKPEDTA
VYYCANGDLRYGPDGYDYRGQGTQVTVSSGGGSCAAA
(SEQ ID NO: 122)
Anti-CD5 VHH319 C-terminal G4SG4S (Conjugation
handle G4SG4S is underlined; conjugation to
delivery vehicle is through C in bold italics;
WT N82 is in bold italics)
QVQLVESGGGLVQPGGSLRLSCAASGFTFSMYSMSWVRQAPGKRLEW
VSSISTGARDTAYADSVKGRFTISRDNADNTLYLHMNNLKPEDTAVY
YCANGDLRYGPDGYDYRGQGTQVTVSSGGGGSGGGGSCAS
(SEQ ID NO: 123)
Anti-CD5 VHH319 Internal S82bC
(Bold underlined N82C substitution)
QVQLVESGGGLVQPGGSLRLSCAASGFTFSMYSMSWVRQAPGKRLEW
VSSISTGARDTAYADSVKGRFTISRDNADNTLYLHMNCLKPEDTAVY
YCANGDLRYGPDGYDYRGQGTQVTVSS (SEQ ID NO: 124)

### Generating Circular RNA

In some aspects, provided herein is a precursor RNA comprising, from 5′ to 3′ end: (i) a 3′ Group I or Group II intron splicing fragment, (ii) a 5′ untranslated region (UTR), (ii) an IRES, (iii) a coding sequence encoding a chimeric antigen receptor chimeric antigen receptor (CAR) comprising an anti-CD19 antibody or antigen-binding fragment thereof, (iv) a 3′ untranslated region (UTR), and (v) a 5′ Group I or Group II intron splicing fragment, wherein the 3′ and 5′ Group I intron splicing fragments are derived from a Group I or Group II intron.

In some aspects, provided herein is a precursor RNA comprising, from 5′ to 3′ end: (i) a 3′ Group I intron splicing fragment, (ii) a 5′ untranslated region (UTR), (iii) an IRES, (iv) a coding sequence encoding a chimeric antigen receptor chimeric antigen receptor (CAR) comprising an anti-CD19 antibody or antigen-binding fragment thereof, (v) a 3′ untranslated region (UTR), and (vi) a 5′ Group I intron splicing fragment, wherein the 3′ and 5′ Group I intron splicing fragments are derived from a Twort-ORF142 Group I intron. In some embodiments, the 3′ UTR comprises a microRNA binding site.

In some embodiments, self-splicing of the 3′ Group I intron fragment and the 5′ Group I intron fragment is capable of generating a circular RNA.

In some aspects, provided herein is a precursor RNA comprising, from 5′ to 3′ end: (i) a 5′ intron splicing fragment and a 5′ exon fragment, (ii) a 5′ untranslated region (UTR), (iii) an IRES, (iv) a coding sequence encoding a chimeric antigen receptor (CAR) comprising an anti-CD19 antibody or antigen-binding fragment thereof, (v) a 3′ untranslated region (UTR), and (vi) a 3′ exon fragment and a 3′ intron splicing fragment, wherein the 5′ and the 3′ intron splicing fragments are derived from a Twort-ORF142 Group I intron, and wherein the 3′ UTR comprises a single microRNA binding site.

In some embodiments, the 5′ intron splicing fragment is derived from the 3′ Twort Group I intron splicing fragment. In some embodiments, the 3′ intron splicing fragment is derived from the 5′ Twort Group I intron splicing fragment.

In some embodiments, the single microRNA binding site is flanked by an upstream spacer and a downstream spacer.

In some embodiments, each of the upstream spacer and the downstream spacer comprise no more than five (5) consecutive adenines (As). In some embodiments, each of the upstream spacer and the downstream spacer do not comprise more than four (4) consecutive adenines (As). In some embodiments, each of the upstream spacer and the downstream spacer do not comprise more than three (3) consecutive adenines (As).

In some embodiments, each of the upstream spacer and the downstream spacer comprise 2, 3, 4 or 5 consecutive adenines. In some embodiments, each of the upstream spacer and the downstream spacer comprise two consecutive adenines. In some embodiments, each of the upstream spacer and the downstream spacer comprise 3 consecutive adenines. In some embodiments, each of the upstream spacer and the downstream spacer comprise 4 consecutive adenines. In some embodiments, each of the upstream spacer and the downstream spacer comprise 5 consecutive adenines.

In some aspects, provided herein is a precursor RNA comprising, from 5′ to 3′ end: (i) a 5′ intron splicing fragment and a 5′ exon fragment, (ii) a 5′ untranslated region (UTR), (iii) an IRES, (iv) a coding sequence encoding a chimeric antigen receptor (CAR) comprising an anti-CD19 antibody or antigen-binding fragment thereof, (v) a 3′ untranslated region (UTR), and (vi) a 3′ exon fragment and a 3′ intron splicing fragment, wherein the 5′ and 3′ intron splicing fragments are derived from a Twort-ORF142 Group I intron, and wherein the 3′ UTR comprises a microRNA binding site flanked by an upstream spacer and a downstream spacer, wherein each of the upstream spacer and the downstream spacer comprise no more than five (5) consecutive adenines (As).

In some embodiments, the sequence of each of the upstream spacer and the downstream spacer has a hydropathy index between −0.6 to −0.3.

In some embodiments, the sequence of the upstream spacer has a hydropathy index of −0.6 to −0.4.

In some embodiments, the sequence of each of the upstream spacer and the downstream spacer has a hydropathy index of −0.6 to −0.5.

In some embodiments, the sequence of each of the upstream spacer and the downstream spacer has a hydropathy index of −0.6. In some embodiments, the sequence of each of the upstream spacer and the downstream spacer has a hydropathy index of −0.5. In some embodiments, the sequence of each of the upstream spacer and the downstream spacer has a hydropathy index of −0.4. In some embodiments, the sequence of each of the upstream spacer and the downstream spacer has a hydropathy index of −0.3.

In some embodiments, each of the upstream spacer and downstream spacer comprise one or more instances of a cytosine (C) interrupting consecutive As.

In some embodiments, each of the upstream spacer and downstream spacer comprise no uracil.

In some embodiments, the upstream spacer sequence and the downstream spacer sequence consist only of As and Cs.

In some embodiments, each of the upstream spacer and downstream spacer 5 a length of not more than 18 nucleotides. In some embodiments, each of the upstream spacer and downstream spacer have a length of 17 nucleotides. In some embodiments, each of the upstream spacer and downstream spacer have a length of 16 nucleotides. In some embodiments, each of the upstream spacer and downstream spacer have a length of 15 nucleotides. In some embodiments, each of the upstream spacer and downstream spacer have a length of 14 nucleotides. In some embodiments, each of the upstream spacer and downstream spacer have a length of 13 nucleotides. In some embodiments, each of the upstream spacer and downstream spacer have a length of 12 nucleotides.

In some embodiments, each of the upstream spacer and downstream spacer have a length of 12-18 nucleotides.

In some embodiments, the upstream spacer and the downstream spacer comprise comparable numbers of nucleotides.

In some embodiments, the microRNA binding site is a microRNA-122 (miR-122) binding site.

In some embodiments, the 3′ UTR and the 3′ exon fragment together comprises SEQ ID NO: 268 or 272. In some embodiments, the 3′ UTR and the 3′ exon fragment together comprises SEQ ID NO: 268. In some embodiments, the 3′ UTR and the 3′ exon fragment together comprises SEQ ID NO: 272.

In some embodiments, the 5′ UTR and the 5′ exon fragment together comprises SEQ ID NO: 265.

In some embodiments, the 5′ UTR and the 5′ exon fragment together comprises SEQ ID NO: 265 and the 3′ UTR and the 3′ exon fragment together comprises SEQ ID NO: 268.

In some embodiments, the IRES comprises a nucleic acid sequence having at least 90% identity to any one of SEQ ID NO: 1-6.

In some embodiments, the IRES comprises a nucleic acid sequence of SEQ ID NO: 1.

In some embodiments, the 5′ intron fragment comprises SEQ ID NO: 276 and the 3′ intron fragment comprises SEQ ID NO: 277.

In some embodiments, the 5′ intron fragment comprises SEQ ID NO: 278 and the 3′ intron fragment comprises SEQ ID NO: 279.

In some embodiments, the 5′ intron fragment comprises SEQ ID NO: 280 and the 3′ intron fragment comprises SEQ ID NO: 281.

In some aspects, provided herein is a precursor RNA comprising, from 5′ to 3′ end: (i) a 5′ intron splicing fragment and a 5′ exon fragment, (ii) a 5′ untranslated region (UTR), (iii) an IRES, (iv) a coding sequence encoding a chimeric antigen receptor (CAR) comprising an anti-CD19 antibody or antigen-binding fragment thereof, (v) a 3′ untranslated region (UTR), and (vi) a 3′ exon fragment and 3′ intron splicing fragment, wherein the 5′ and the 3′ intron splicing fragments are derived from a Twort-ORF142 Group I intron, wherein the 3′ UTR comprises a microRNA binding site flanked by an upstream spacer and a downstream spacer, and wherein each of the upstream spacer and the downstream spacer comprise no more than five (5) consecutive adenines (As).

In some embodiments, each of the upstream spacer and the downstream spacer comprise no more than four (4) consecutive adenines (As).

In some embodiments, each of the upstream spacer and the downstream spacer comprise no more than three (3) consecutive adenines (As).

In some embodiments, each of the upstream spacer and the downstream spacer comprise one or more instances of a cytosine (C) interrupting consecutive As.

In some embodiments, each of the upstream spacer and the downstream spacer comprise no uracil.

In some embodiments, each of the upstream spacer and the downstream spacer comprise 12-18 nucleotides.

In some embodiments, each of the upstream spacer and downstream spacer have a length of 12-15 nucleotides.

In some embodiments, the upstream spacer and the downstream spacer do not differ in length by more than 4 nucleotides.

In some embodiments, the upstream spacer and the downstream spacer do not differ in length by more than 3 nucleotides.

In some embodiments, the upstream spacer and the downstream spacer do not differ in length by more than 2 nucleotides.

In some embodiments, the microRNA binding site is a microRNA-122 (miR-122) binding site.

In some embodiments, the 5′ intron splicing fragment comprises SEQ ID NO: 381.

In some embodiments, the 5′ exon fragment comprises SEQ ID NO: 382.

In some embodiments, the 5′ UTR comprises SEQ ID NO: 348.

In some embodiments, the IRES comprises a sequence having at least 90% identity to any one of SEQ ID NO: 1-6.

In some embodiments, the IRES comprises the sequence of SEQ ID NO: 1.

In some embodiments, the coding sequence encoding the CAR comprising the anti-CD19 antibody or antigen-binding fragment thereof comprises the sequence of SEQ ID NO: 355.

In some embodiments, the 3′ UTR comprises SEQ ID NO: 349 or 350.

In some embodiments, the 3′ exon fragment comprises SEQ ID NO: 389.

In some embodiments, the 3′ intron splicing fragment comprises SEQ ID NO: 390.

In some embodiments, the 5′ UTR comprises SEQ ID NO: 348 and the 3′ UTR comprises SEQ ID NO: 349.

In some embodiments, the 5′ intron splicing fragment and the 5′ exon fragment together comprises SEQ ID NO: 276 and the 3′ exon fragment and the 3′ intron fragment together comprises SEQ ID NO: 277.

In some aspects, provided herein is a precursor RNA comprising, from 5′ to 3′ end: (i) a 5′ intron fragment and a 5′ exon fragment together comprising SEQ ID NO: 276, (ii) a 5′ untranslated region (UTR), (iii) an IRES, (iv) a coding sequence encoding a chimeric antigen receptor (CAR) comprising an anti-CD19 antibody or antigen-binding fragment thereof, (v) a 3′ untranslated region (UTR), and (vi) a 3′ exon fragment and a 3′ intron fragment together comprising SEQ ID NO: 277.

In some embodiments, provided herein is a circular RNA generated using the precursor RNA of the present disclosure.

In some embodiments, provided herein is a method of making circular RNA by using the precursor RNA of the present disclosure.

In some embodiments, provided herein is a circRNA circularized using the methods of the present disclosure.

In some embodiments, the nucleic acid precursor of the circRNA molecule is synthetic DNA. In some embodiments, the nucleic acid precursor of the circRNA molecule is synthetic RNA. In some embodiments, the nucleic acid is chemically synthesized. In some embodiments, the nucleic acid is synthesized in synthesizer machines using nucleotide triphosphate derivatives known as phosphoramidites, which are building blocks of linear oligonucleotides. Nucleoside phosphoramidites use inert substituents to protect reactive moieties such as hydroxyl and amino groups from undesirable reactions and promote phosphodiester bond formation leading to greater homogenous yields. Once synthesis is complete, these groups are removed to generate DNA or RNA oligonucleotides of high purity.

In some embodiments, the nucleic acid is in vitro transcribed RNA. An in vitro transcription (IVT) reaction typically comprises a double-stranded DNA (dsDNA) template, ribonucleotide triphosphates, and a DNA-dependent RNA polymerase. In some embodiments, the DNA-dependent RNA polymerase is derived from bacteriophage. In some embodiments, the DNA-dependent RNA polymerase includes but is not limited to a T7 RNA polymerase, SP6 RNA polymerase, or T3 RNA polymerase. In some embodiments, the polymerase is a T7 RNA polymerase.

In some embodiments, the nucleic acid is a DNA template. The DNA template contains a promoter sequence to which the polymerase binds and catalyzes downstream transcription. In some embodiments, the promoter is about 20-40 nucleotides long. In some embodiments, the DNA template is a double-stranded PCR product. In some embodiments, the DNA template is a linearized plasmid containing a promoter upstream of the DNA sequence to be transcribed.

In some embodiments, the RNA precursor molecule is circularized into circular RNA. Circular RNA may be produced via any method known in the art. In some embodiments, the circular RNA is created via back-splicing using a self-splicing Group I intron. In some embodiments, the circular RNA is created using a self-splicing Group II intron.

In some embodiments, viral vectors are used to package the constructs for producing the nucleic acids and/or circRNA molecules described herein. In some embodiments, AAV vectors are used to construct the nucleic acids and/or circRNA molecules described herein. In other embodiments, non-viral vectors such as plasmids, cosmids and artificial chromosomes are used to construct the nucleic acids and/or circRNA molecules described herein.

### Self-Splicing Introns

In some embodiments, the nucleic acid molecule described herein comprises permuted self-splicing intron sequences. The permutation generates an upstream (i.e., 5′ end) intron sequence corresponding to a 3′ intron splicing fragment of a self-splicing Group I or Group II intron and a downstream (i.e., 3′ end) intron sequence corresponding to a 5′ intron splicing fragment of the self-splicing intron. As used herein, the 3′ intron splicing fragment and the 3′ intron fragment are used interchangeably. As used herein, the 5′ intron splicing fragment and the 5′ intron fragment are used interchangeably. The intron sequences flank the RNA sequence of interest to be circularized.

Group I introns have autocatalytic activity (acting as ribozymes), i.e., are self-splicing. Group I introns splice themselves out without assistance from the spliceosome or other proteins, resulting in joining of the flanking exons and circularization of the intervening intron to produce an intronic circRNA. Group I introns can be found naturally within the rRNA, tRNA, and mRNA genes of bacteria and non-metazoan eukaryotes. A general discussion of the catalytic activity of Group I introns can be found in the review article by Hausner et al., Mobile DNA, vol 5(8) (2014).

Group II introns constitute another ribozyme self-splicing system with a mechanism similar to that of both Group I introns and pre-mRNA introns. Group II introns are naturally found in the bacteria and organelle genomes of lower eukaryotes. A general discussion of the catalytic activity of Group II introns can be found in the review article by Pyle, Annu Rev Biophys. 5(45): 183-205) (2016)).

In some embodiments, the nucleic acid molecule for making a circular RNA comprises an upstream intron sequence and a downstream intron sequence generated from the cut site or splitting position (“cut site”, “permutation site”, and “splitting position” are used interchangeably herein), wherein the cut site retains the structural integrity of the intron. As used herein, the term “catalytic core” refers to an intron region (e.g., the internal stem structure) needed for the intron to self-splice, which is well-understood in the art. Typically, the catalytic core of an intron, particularly, a Group I intron, is a highly conserved small region of about 70 nucleotides composed of paired regions (e.g., P1-P6) that form elongated domains (e.g., helical domains). More detailed description can be found in, e.g., Michel and Westhof, J. Mol. Bio., 1990, 216: 585-610; Luptak and Doudna, Nucleic Acids Research, 2004, 32(7): 2272-80, the contents of which are incorporated by reference herein. In some embodiments, the cut site is designed to retain the catalytic core of the intron, which is a region of nucleotides containing sequences and structures needed for the intron to self-splice. In some embodiments, a suitable cut site is chosen outside the catalytic core of the intron such that the catalytic core may retain its structural integrity.

Accordingly, in some embodiments, the present disclosure provides a nucleic acid molecule for making circular RNA comprising an upstream intron sequence and a downstream intron sequence corresponding to a 3′ splicing intron fragment and a 5′ splicing intron fragment derived from a Group I intron or from a Group II intron, respectively. Exemplary Group I introns include but are not limited to the Twort-ORF142 intron. In some embodiments, the upstream intron sequences within the nucleic acid molecule for making a circular RNA correspond to a 3′ splicing fragment of the Twort-ORF142 intron. In some embodiments, the downstream intron sequences within the nucleic acid molecule for making a circular RNA correspond to a 5′ splicing fragment of the Twort-ORF142 intron.

### Exemplary Group I Intron Sequences

In some embodiments, the nucleic acid molecule described herein comprises permuted intron sequences corresponding to a Twort-ORF142 intron. Exemplary Group I introns include but are not limited to the Twort-ORF142 intron comprising SEQ ID NO: 273.

In some embodiments, the upstream and downstream intron sequences within the nucleic acid molecule for making a circular RNA correspond to a 3′ splicing fragment and a 5′ splicing fragment of the Twort-ORF142 intron, respectively.

In some embodiments, the upstream and downstream intron sequences each correspond to the 3′ and 5′ intron splicing fragments of SEQ ID NO: 273.

As used herein, the upstream intron fragment comprises 5′ intron splicing fragment and 5′ exon fragment, and the downstream intron fragment comprises 3′ exon fragment and 3′ intron splicing fragment.

The Twort-ORF142 intron sequence includes nucleotides 1055-1336 of the open reading frame Twort-ORF142 (unknown gene) in the genome of Staphylococcus phage Twort (GenBank: AF132670.1) (Landthaler and Shub, PNAS, 1999, 96 (12), 7005-7010):

(SEQ ID NO: 273)
AACTACTGAAAGCATAAATAATTGTGCCTTTATACAGTAATGTATA
TCGAAAAATCCTCTAATTCAGGGAACACCTAAACAAACTAAGATG
TAGGCAATCCTGAGCTAAGCTCTTAGTAATAAGAGAAAGTGCAAC
GACTATTCCGATAGGAAGTAGGGTCAAGTGACTCGAAATGGGGAT
TACCCTTCTAGGGTAGTGATATAGTCTGAACATATATGGAAACAT
ATAGAAGGATAGGAGTAACGAACCTATTCGTAACATAATTGAACT
TTTAGTTATTT.

In some embodiments, the nucleic acid molecule for making a circular RNA comprises the upstream intron sequence comprising a sequence corresponding to positions 119 to 282 of SEQ ID NO: 273, and the downstream intron sequence comprising a sequence corresponding to positions 1 to 118 of SEQ ID NO: 273. In one non-limiting example, the upstream intron sequence corresponds to positions 119 to 282 of SEQ ID NO: 273, and the downstream intron sequence corresponds to positions 1 to 118 of SEQ ID NO: 273.

In some embodiments, the nucleic acid molecule for making a circular RNA comprises the upstream intron sequence of SEQ ID NO: 276 and the downstream intron sequence of SEQ ID NO: 277.

In some embodiments, the nucleic acid molecule for making a circular RNA comprises an upstream intron sequence that is at least 75%, 80%, 85%, 90%, 95%, 96%, 97%, 98%, or 99% identical to SEQ ID NO: 276, and a downstream intron sequence that is at least 75%, 80%, 85%, 90%, 95%, 96%, 97%, 98%, or 99% identical to SEQ ID NO: 277.

In some embodiments, the upstream intron sequence comprises the sequence of SEQ ID NO: 276, and wherein the downstream intron sequence comprises the sequence of SEQ ID NO: 277.

In some embodiments, the upstream intron fragment comprises a sequence corresponding to positions 147 to 282 of SEQ ID NO: 273, and the downstream intron fragment comprises a sequence corresponding to positions 1 to 146 of SEQ ID NO: 273. In one non-limiting example, the upstream intron sequence corresponds to positions 147 to 282 of SEQ ID NO: 273, and the downstream intron sequence corresponds to positions 1 to 146 of SEQ ID NO: 273.

In some embodiments, the upstream intron sequence comprises a sequence corresponding to positions 140 to 282 of SEQ ID NO. 273, and the downstream intron sequence comprises a sequence corresponding to positions 1 to 139 of SEQ ID NO: 273. In some embodiments, the upstream intron sequence comprises a sequence corresponding to positions 147 to 282 of SEQ ID NO: 273, and the downstream intron sequence comprises a sequence corresponding to positions 1 to 146 of SEQ ID NO: 273.

In some embodiments, the upstream intron fragment comprises the sequence of SEQ ID NO: 280 and the downstream intron fragment comprises the sequence of SEQ ID NO: 281.

In some embodiments, the upstream intron sequence is at least 75%, 80%, 85%, 90%, 95%, 96%, 97%, 98%, or 99% identical to SEQ ID NO: 280. In some embodiments, the downstream intron sequence is at least 75%, 80%, 85%, 90%, 95%, 96%, 97%, 98%, or 99% identical to SEQ ID NO: 281.

In some embodiments, the nucleic acid molecule for making a circular RNA comprises the upstream intron sequence comprising a sequence corresponding to positions 140 to 282 of SEQ ID NO: 273, and the downstream intron sequence comprising a sequence corresponding to positions 1 to 139 of SEQ ID NO: 273. In one non-limiting example, the upstream intron sequence corresponds to positions 140 to 282 of SEQ ID NO: 273, and the downstream intron sequence corresponds to positions 1 to 139 of SEQ ID NO: 273.

In some embodiments, the upstream intron fragment comprises the sequence of SEQ ID NO: 278 and the downstream intron fragment comprises the sequence of SEQ ID NO: 279.

In some embodiments, the upstream intron sequence is at least 75%, 80%, 85%, 90%, 95%, 96%, 97%, 98%, or 99% identical to SEQ ID NO: 278. In some embodiments, the downstream intron sequence is at least 75%, 80%, 85%, 90%, 95%, 96%, 97%, 98%, or 99% identical to SEQ ID NO: 279.

In some embodiments, the nucleic acid molecule for making a circular RNA comprises an upstream intron sequence and a downstream intron sequence generated from a cut site or splitting position (“cut site” and “splitting position” are used interchangeably herein), wherein the cut site retains the structural integrity of the intron. As used herein, the term “catalytic core” refers to an intron region (e.g., the internal stem structure) needed for the intron to self-splice, which is well-understood in the art. Typically, the catalytic core of an intron, particularly, a Group I intron, is a highly conserved small region of about 70 nucleotides composed of paired regions (e.g., P1-P6) that form elongated domains (e.g., helical domains). More detailed description can be found in, e.g., Michel and Westhof, J. Mol. Bio., 1990, 216: 585-610; which is incorporated by reference herein. In some embodiments, the cut site is designed to retain the catalytic core of the intron, which is a region of nucleotides containing sequences and structures needed for the intron to self-splice. In some embodiments, a suitable cut site is chosen outside the catalytic core of the intron such that the catalytic core may retain its structural integrity.

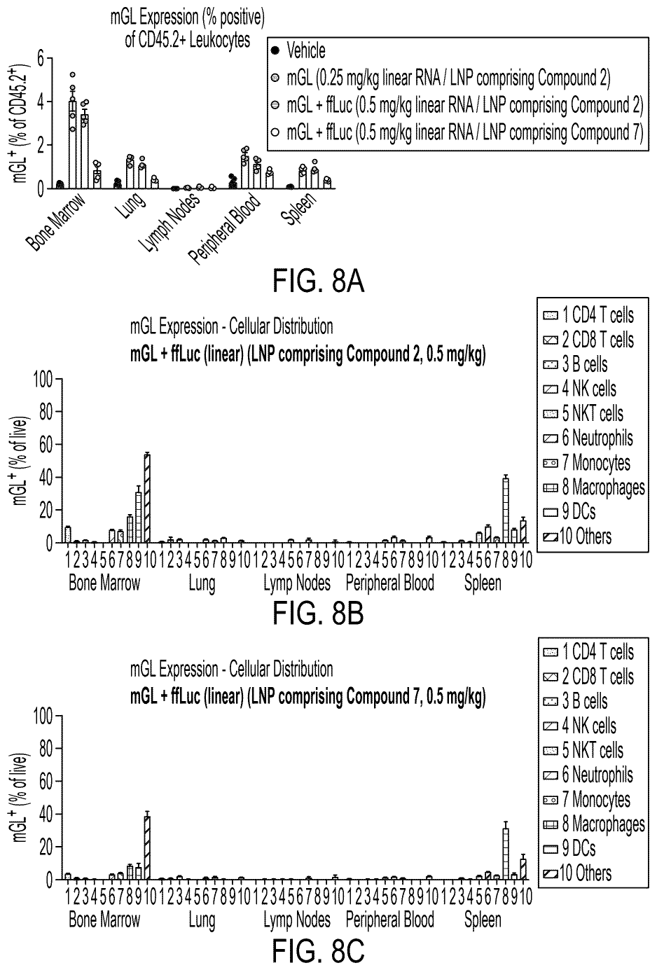

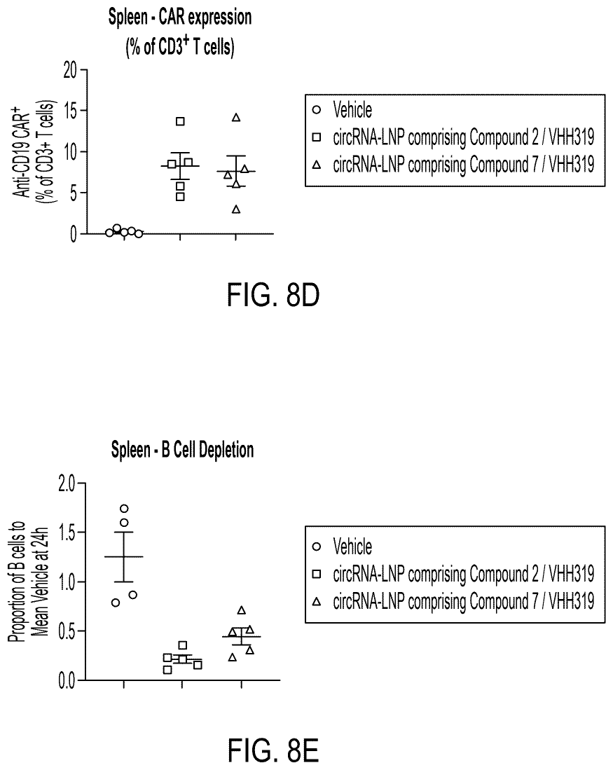

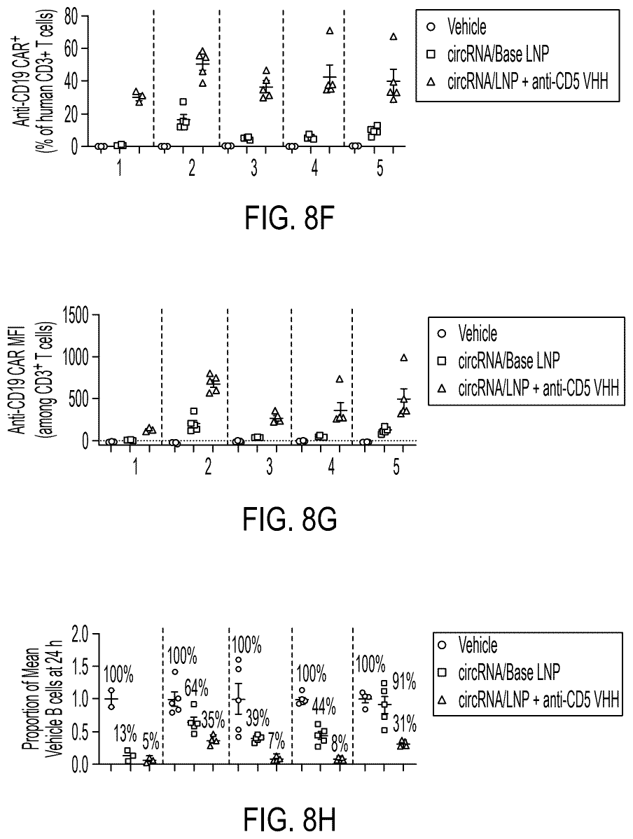

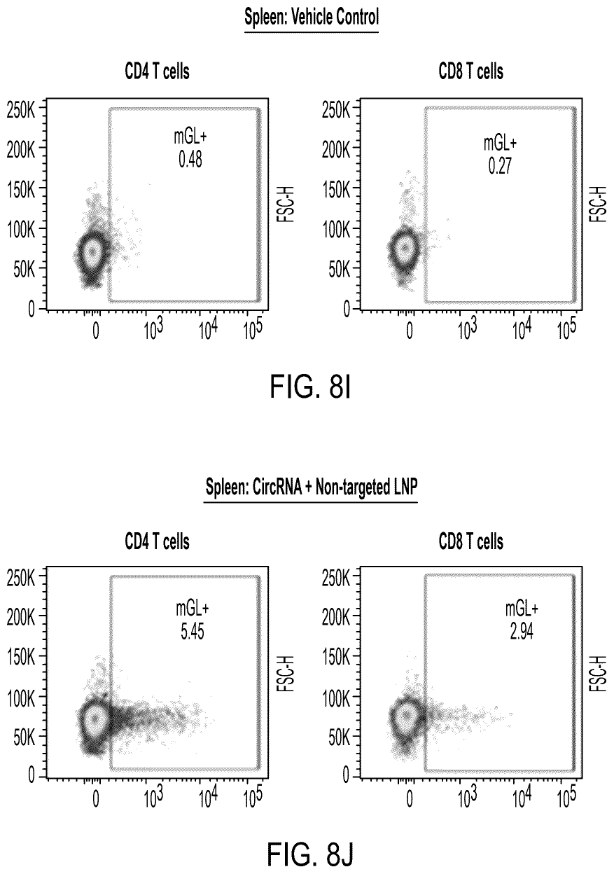

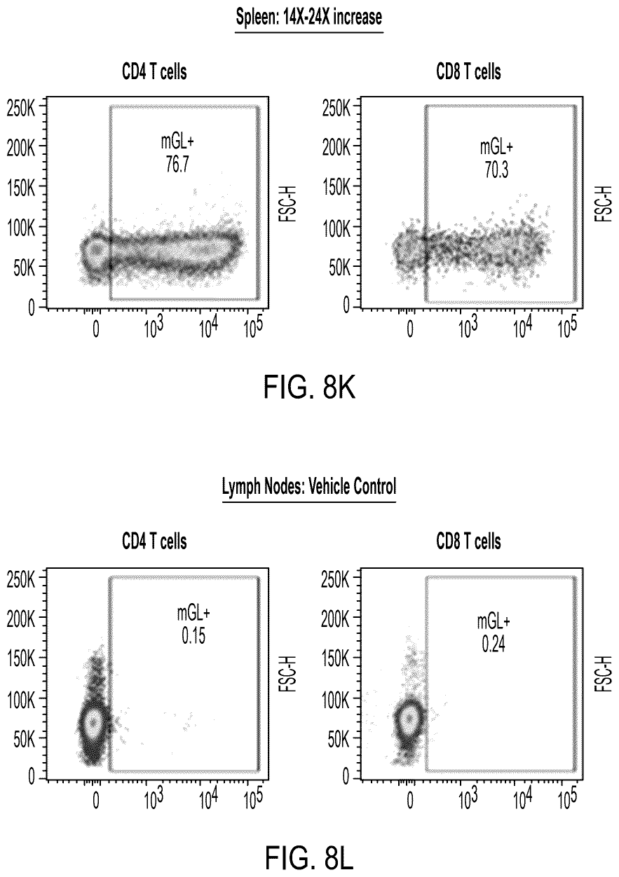

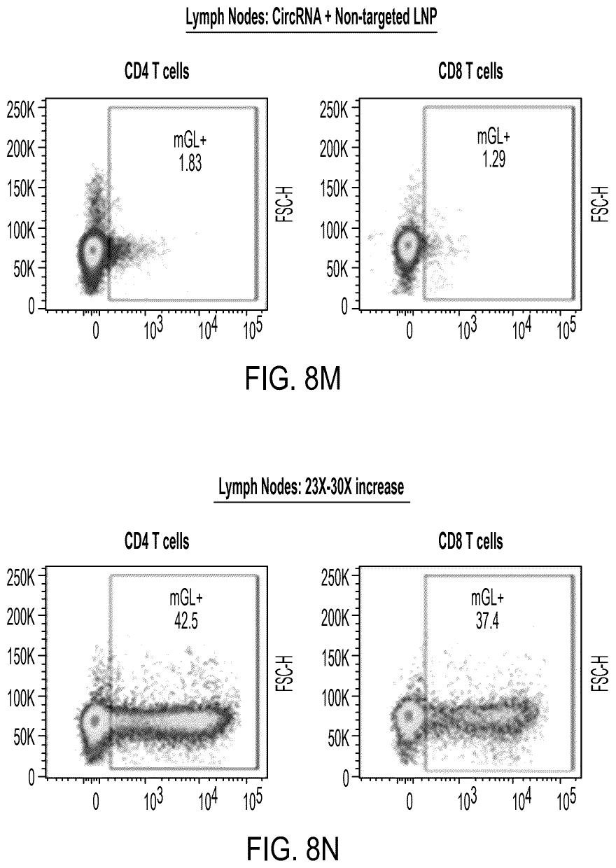

In some embodiments, the nucleic acid molecule for making a circular RNA comprises an upstream intron sequence and a downstream intron sequence derived from a Twort intron generated from a cut site or splitting position, wherein the cut site is between any two residues within a region defined by P1, P2, P3, P4, P5, P5a, P6, P6a, P7, P7.1, P7.2, P8, P9, P9.0, P9.1, P10 or P12 as shown, for example, in FIG. 8 in Landthaler and Shub, PNAS, 1999, 96 (12), 7005-7010), which is incorporated herein by reference.

In some embodiments, the cut site is a V1 cut site, a V2 cut site or a V3 cut site. In some embodiments, the cut site is a V1 cut site between any two residues in a P7 region. In some embodiments, the cut site is a V2 cut site between any two residues in a P6a region. In some embodiments, the cut site is a V3 cut site between any two residues in a P7.1 region.

In some embodiments, the V1, V2 and/or V3 cut sites are between any two residues within a loop. In some embodiments, the V1, V2 and/or V3 cut sites are not within an internal stem structure of the intron. In some embodiments, a V1 site is between adenine and uracil (A/U) in a P7 region.

In some embodiments, a V2 site is between any two residues in a loop comprising AGUAAU in a P6a region. In some embodiments, a V2 cute site is between adenine and guanine (A/G) within the loop of the intron. In some embodiments, a V2 site is between guanine and uracil (G/U) within the loop of the intron. In some embodiments, a V2 cut site between uracil and adenine (U/A) within the loop of the intron. In some embodiments, a V2 sit is between two adenines within the loop of the intron. In some embodiments, a V2 site is between adenine at position 5 of the loop and uracil at position 6 of the loop. In some embodiments, a V2 cut site between uracil at position 3 of the loop and adenine at position 4 (U/A) of the loop. In some embodiments, a V2 cut site is at an equivalent position in a loop comprising a complementary loop sequence to AGUAAU. In some embodiments, a V2 site is at an equivalent position in a loop comprising one or more variations in the loop sequence of AGUAAU. In some embodiments, a V2 cut site is at an equivalent position in a loop comprising 1, 2, 3, 4, 5 or 6 variations in the loop sequence of AGUAAU. In some embodiments, a V2 site is at a position between a stem residue and the first or last residue of the loop sequence of AGUAAU.

In some embodiments, a V3 site is between any two residues in a loop comprising GAUA in a P7.1 region. In some embodiments, a V3 site is between guanine and adenine (G/A) within a loop of the intron. In some embodiments, a V3 cut site is between adenine and uracil (A/U) within a loop of the intron. In some embodiments, a V3 site is between uracil and adenine (U/A) within a loop of the intron. In some embodiments, a V3 site is at an equivalent position in a loop comprising a complementary loop sequence to GAUA. In some embodiments, a V3 cut site is at an equivalent position in a loop comprising one or more variations in the loop sequence of GAUA. In some embodiments, a V3 cut site is at an equivalent position in a loop comprising 1, 2, 3 or 4 variations in the loop sequence of GAUA. In some embodiments, a V3 cut site is at a position between a stem residue and the first or last residue of the loop sequence of GAUA.

In some embodiments, the nucleic acid molecules comprise intron sequence fragments listed in Table 13A.

TABLE 13A
Exemplary intron sequence fragments

| | | Sequence (5′-3′ end) |
|---|---|---|
| Twort- | Upstream | AATAAGAGAAAGTGCAACGACTATTCCGATAGGAAGTAGGGTCAAGT |
| ORF142 | intron | GACTCGAAATGGGGATTACCCTTCTAGGGTAGTGATATAGTCTGAAC |
| intron-V2 | sequence | ATATATGGAAACATATAGAAGGATAGGAGTAACGAACCTATTCGTAA |
| | | CATAATTGAACTTTTAGTTATTT (SEQ ID NO: 276) |
| | Downstream | AACTACTGAAAGCATAAATAATTGTGCCTTTATACAGTAATGTATAT |
| | intron | CGAAAAATCCTCTAATTCAGGGAACACCTAAACAAACTAAGATGTAG |
| | sequence | GCAATCCTGAGCTAAGCTCTTAGT (SEQ ID NO: 277) |
| Twort- | Upstream | TATTCCGATAGGAAGTAGGGTCAAGTGACTCGAAATGGGGATTACCC |
| ORF142 | intron | TTCTAGGGTAGTGATATAGTCTGAACATATATGGAAACATATAGAAG |
| intron-V1 | sequence | GATAGGAGTAACGAACCTATTCGTAACATAATTGAACTTTTAGTTAT |
| | | TTGAGCTCTCGGCGACCATTTGTGTGGT (SEQ ID NO: 278) |
| | Downstream | AACTACTGAAAGCATAAATAATTGTGCCTTTATACAGTAATGTATAT |
| | intron | CGAAAAATCCTCTAATTCAGGGAACACCTAAACAAACTAAGATGTAG |
| | sequence | GCAATCCTGAGCTAAGCTCTTAGTAATAAGAGAAAGTGCAACGAC |
| | | (SEQ ID NO: 279) |
| Twort- | Upstream | TAGGAAGTAGGGTCAAGTGACTCGAAATGGGGATTACCCTTCTAGGG |
| ORF142 | intron | TAGTGATATAGTCTGAACATATATGGAAACATATAGAAGGATAGGAG |
| intron-V3 | sequence | TAACGAACCTATTCGTAACATAATTG AACTTTTAGTTATTT (SEQ |
| | | ID NO: 280) |
| | Downstream | AACTACTGAAAGCATAAATAATTGTGCCTTTATACAGTAATGTATAT |
| | intron | CGAAAAATCCTCTAATTCAGGGAACACCTAAACAAACTAAGATGTAG |
| | sequence | GCAATCCTGAGCTAAGCTCTTAGTAATAAGAGAAAGTGCAACGACTA |
| | | TTCCGA (SEQ ID NO: 281) |

In some embodiments, an exemplary precursor RNA is transcribed from a DNA template as depicted in Table 13B. The components of the DNA template are described in 5′ to 3′ order and the corresponding precursor RNA components are also listed. The precursor RNA is circularized to generate circular RNA containing components described herein (see, e.g., Table 7D).

TABLE 13B
Components of Exemplary DNA Template Used to Make Precursor RNA and Exemplary
Precursor RNA (in 5′ to 3′ order from top to bottom) Used to Generate Circular RNA

| Component | | |
|---|---|---|
| Name | DNA Template Component¹ | RNA Precursor Component² |
| T7 RNA | GGCCAGTGAATTGtaatacg | GGGAGAAU (No SEQ ID designated for |
| Polymerase | actcactataGGGAGAAT (SEQ ID NO: | less than 10 nucleotides) |
| Promoter and | 365) | |
| flanking | | |
| sequences³ | | |
| pA25 Spacer | AAAAAAAAAAAAAAAAAAAAAAAAA | AAAAAAAAAAAAAAAAAAAAAAAAA (SEQ ID NO: |
| | (SEQ ID NO: 295) | 366) |
| Miscellaneous | TCACCGACCGACCGGT (SEQ ID NO: | UCACCGACCGACCGGU (SEQ ID NO: 380) |
| Residual | 367) | |
| Sequence | | |
| 5′ intron | AATAAGAGAAAGTGCAACGACTATTCCGAT | AAUAAGAGAAAGUGCAACGACUAUUCCGAUAGGAAGU |
| fragment⁶ | AGGAAGTAGGGTCAAGTGACTCGAAATGGG | AGGGUCAAGUGACUCGAAAUGGGGAUUACCCUUCUAG |
| | GATTACCCTTCTAGGGTAGTGATATAGTCT | GGUAGUGAUAUAGUCUGAACAUAUAUGGAAACAUAUA |
| | GAACATATATGGAAACATATAGAAGGATAG | GAAGGAUAGGAGUAACGAACCUAUUCGUAACAUAAUU |
| | GAGTAACGAACCTATTCGTAACATAATTG | G (SEQ ID NO: 381) |
| | (SEQ ID NO: 368) | |
| 5′ exon | aacttttagttattt (SEQ ID NO: | aacuuuuuuuuu (SEQ ID NO: 382) |
| fragment⁶ | 369) | |
| Internal | TCGGCGACCATTTGTGTGGT (SEQ ID | UCGGCGACCAUUUGUGUGGU (SEQ ID NO: 383) |
| Homology | NO: 370) | |
| Element | | |
| (Upstream)⁴ | | |
| 5′ UTR | GAGCTC (No SEQ ID designated | GAGCUC (No SEQ ID designated for less |
| Cloning Site | for less than 10 nucleotides) | than 10 nucleotides) |
| 1⁴ | | |
| Random | TAAGTGTTGATCAGCGGGCGCAGCTGCATA | UAAGUGUUGAUCAGCGGGCGCAGCUGCAUAUGACCUC |
| Insert⁴ | TGACCTCTGCGTGTGCCTGG (SEQ ID | UGCGUGUGCCUGG (SEQ ID NO: 384) |
| | NO: 371) | |
| 3x Frame | TAATTGACTAA (SEQ ID NO: 372) | UAAUUGACUAA (SEQ ID NO: 385) |
| Stop Codon⁴ | | |
| 5′ UTR | GATATC (No SEQ ID designated | GAUAUC (No SEQ ID designated for less |
| Cloning Site | for less than 10 nucleotides) | than 10 nucleotides) |
| 2⁴ | | |
| IRES263 | TTACTCAGCGTAACTACTCCGGGTTACGTG | UUACUCAGCGUAACUACUCCGGGUUACGUGAUGAAGA |
| | ATGAAGAAGAGGCTACGGAGATTCTCGGGC | AGAGGCUACGGAGAUUCUCGGGCUACGGCCCUGGAGC |
| | TACGGCCCTGGAGCCACTCCGGCTCCTAAA | CACUCCGGCUCCUAAAGAUUUAGAAGUUUGAGCACAC |
| | GATTTAGAAGTTTGAGCACACCCGCCCACT | CCGCCCACUAGGGCCCCCCAUCCAGGGGGGCAACGGG |
| | AGGGCCCCCCATCCAGGGGGGCAACGGGCA | CAAGCACUUCUGUUUCCCCGGUAUGAUCUGAUAGGCU |
| | AGCACTTCTGTTTCCCCGGTATGATCTGAT | GUAACCACGGCUGAAACAGAGAUUAUCGUUAUCCGCU |
| | AGGCTGTAACCACGGCTGAAACAGAGATTA | UCACUACUUCGAGAAGCCUAGUAAUGAUGGGUGAAAU |
| | TCGTTATCCGCTTCACTACTTCGAGAAGCC | UGAAUCCGUUGAUCCGGUGUCUCCCCCACACCAGAAA |
| | TAGTAATGATGGGTGAAATTGAATCCGTTG | CUCAUGAUGAGGGUUGCCAUCCCGGCUACGGCGACGU |
| | ATCCGGTGTCTCCCCCACACCAGAAACTCA | AGCGGGCAUCCCUGCGCUGGCAUGAGGCCUCUUAGGA |
| | TGATGAGGGTTGCCATCCCGGCTACGGCGA | GGACGGAUGAUAUGGAUCUUGUCGUGAAGAGCCUAUU |
| | CGTAGCGGGCATCCCTGCGCTGGCATGAGG | GAGCUAGUGUCGACUCCUCCGCCCCCGUGAAUGCGGC |
| | CCTCTTAGGAGGACGGATGATATGGATCTT | UAAUCCUAACCCCGGAGCAGGUGGGUCCAAUCCAGGG |
| | GTCGTGAAGAGCCTATTGAGCTAGTGTCGA | CCUGGCCUGUCGUAAUGCGUAAGUCUGGGACGGAACC |
| | CTCCTCCGCCCCCGTGAATGCGGCTAATCC | GACUACUUUCGGGAAGGCGUGUUUCCAUUUGUUCAUU |
| | TAACCCCGGAGCAGGTGGGTCCAATCCAGG | AUUUGUGUGUUUAUGGUGACAACUCUGGGUAAACGUU |
| | GCCTGGCCTGTCGTAATGCGTAAGTCTGGG | CUAUUGCGUUUAUUGAGAGAUUCCCAACAAUUGAACA |
| | ACGGAACCGACTACTTTCGGGAAGGCGTGT | AACGAGAACUACCUGUUUUAUUAAAUUUACACAGAGA |
| | TTCCATTTGTTCATTATTTGTGTGTTTATG | AGAAUUACA (SEQ ID NO: 354) |
| | GTGACAACTCTGGGTAAACGTTCTATTGCG | |
| | TTTATTGAGAGATTCCCAACAATTGAACAA | |
| | ACGAGAACTACCTGTTTTATTAAATTTACA | |
| | CAGAGAAGAATTACA (SEQ ID NO: | |
| | 296) | |
| Codon- | ATGGCTCTGCCTGTGACAGCTCTGCTGCTG | AUGGCUCUGCCUGUGACAGCUCUGCUGCUGCCUCUGG |
| optimized | CCTCTGGCTCTGCTTCTGCATGCCGCCAGA | CUCUGCUUCUGCAUGCCGCCAGACCUGACAUCCAGAU |
| coding | CCTGACATCCAGATGACCCAGACAACCAGC | GACCCAGACAACCAGCAGCCUGUCUGCCAGCCUGGGC |
| sequence | AGCCTGTCTGCCAGCCTGGGCGACAGAGTG | GACAGAGUGACCAUCAGCUGUAGAGCCAGCCAGGACA |
| (Open | ACCATCAGCTGTAGAGCCAGCCAGGACATC | UCAGCAAGUACCUGAACUGGUAUCAGCAGAAACCCGA |
| reading | AGCAAGTACCTGAACTGGTATCAGCAGAAA | CGGCACCGUGAAGCUGCUGAUCUACCACACCAGCAGA |
| frame) | CCCGACGGCACCGTGAAGCTGCTGATCTAC | CUGCACAGCGGCGUGCCAUCCAGAUUUUCUGGCAGCG |
| | CACACCAGCAGACTGCACAGCGGCGTGCCA | GCUCUGGCACCGACUACAGCCUGACAAUCAGCAACCU |
| | TCCAGATTTTCTGGCAGCGGCTCTGGCACC | GGAACAAGAGGAUAUCGCUACCUACUUCUGCCAGCAA |
| | GACTACAGCCTGACAATCAGCAACCTGGAA | GGCAACACCCUGCCUUACACAUUCGGCGGAGGCACCA |
| | CAAGAGGATATCGCTACCTACTTCTGCCAG | AGCUGGAAAUCACAGGUGGCGGAGGAUCUGGCGGAGG |
| | CAAGGCAACACCCTGCCTTACACATTCGGC | UGGAAGCGGAGGCGGUGGAUCUGAAGUGAAACUGCAA |
| | GGAGGCACCAAGCTGGAAATCACAGGTGGC | GAGUCUGGCCCUGGCCUGGUGGCUCCAUCUCAGUCUC |
| | GGAGGATCTGGCGGAGGTGGAAGCGGAGGC | UGAGCGUGACCUGUACCGUCAGCGGAGUGUCUCUGCC |
| | GGTGGATCTGAAGTGAAACTGCAAGAGTCT | UGAUUACGGCGUGUCCUGGAUCAGACAGCCUCCUAGA |
| | GGCCCTGGCCTGGTGGCTCCATCTCAGTCT | AAAGGCCUGGAAUGGCUGGGCGUGAUCUGGGGCAGCG |
| | CTGAGCGTGACCTGTACCGTCAGCGGAGTG | AGACAACCUACUACAACAGCGCCCUGAAGUCCAGGCU |
| | TCTCTGCCTGATTACGGCGTGTCCTGGATC | GACCAUCAUCAAGGACAACUCCAAGAGCCAGGUGUUC |
| | AGACAGCCTCCTAGAAAAGGCCTGGAATGG | CUGAAGAUGAACAGCCUGCAGACCGACGACACCGCCA |
| | CTGGGCGTGATCTGGGGCAGCGAGACAACC | UCUACUACUGCGCCAAGCACUACUACUACGGCGGCAG |
| | TACTACAACAGCGCCCTGAAGTCCAGGCTG | CUACGCCAUGGAUUACUGGGGCCAGGGAACCAGCGUG |
| | ACCATCATCAAGGACAACTCCAAGAGCCAG | ACCGUGUCUAGCACAACAACCCCUGCUCCUAGACCUC |
| | GTGTTCCTGAAGATGAACAGCCTGCAGACC | CUACACCAGCUCCUACAAUCGCCAGCCAGCCUCUGUC |
| | GACGACACCGCCATCTACTACTGCGCCAAG | UCUGAGGCCAGAGGCUUGUAGACCUGCUGCUGGCGGA |
| | CACTACTACTACGGCGGCAGCTACGCCATG | GCUGUGCACACAAGAGGCCUGGAUUUCGCCUGCGACA |
| | GATTACTGGGGCCAGGGAACCAGCGTGACC | UCUACAUCUGGGCCCCUCUGGCUGGAACAUGUGGCGU |
| | GTGTCTAGCACAACAACCCCTGCTCCTAGA | GCUGCUGCUGAGCCUGGUCAUCACCCUGUAUUGCAAG |
| | CCTCCTACACCAGCTCCTACAATCGCCAGC | AGAGGCCGGAAGAAGCUGCUCUACAUCUUCAAGCAGC |
| | CAGCCTCTGTCTCTGAGGCCAGAGGCTTGT | CCUUCAUGAGGCCCGUGCAGACCACACAAGAAGAGGA |
| | AGACCTGCTGCTGGCGGAGCTGTGCACACA | CGGCUGCUCCUGCAGAUUCCCCGAGGAAGAGGAAGGC |
| | AGAGGCCTGGATTTCGCCTGCGACATCTAC | GGCUGCGAGCUGAGAGUGAAGUUCAGCAGAAGCGCUG |
| | ATCTGGGCCCCTCTGGCTGGAACATGTGGC | ACGCCCCUGCUUACCAGCAGGGACAGAACCAGCUGUA |
| | GTGCTGCTGCTGAGCCTGGTCATCACCCTG | CAACGAGCUGAACCUGGGGAGAAGAGAAGAGUACGAC |
| | TATTGCAAGAGAGGCCGGAAGAAGCTGCTC | GUGCUGGACAAGCGGAGAGGCAGAGAUCCUGAGAUGG |
| | TACATCTTCAAGCAGCCCTTCATGAGGCCC | GCGGCAAGCCCAGAAGAAAGAACCCUCAAGAGGGCCU |
| | GTGCAGACCACACAAGAAGAGGACGGCTGC | GUAUAAUGAGCUGCAGAAAGACAAGAUGGCCGAGGCC |
| | TCCTGCAGATTCCCCGAGGAAGAGGAAGGC | UACAGCGAGAUCGGAAUGAAGGGCGAGCGCAGAAGAG |
| | GGCTGCGAGCTGAGAGTGAAGTTCAGCAGA | GCAAGGGACACGAUGGACUGUACCAGGGCCUGAGCAC |
| | AGCGCTGACGCCCCTGCTTACCAGCAGGGA | AGCCACCAAGGACACAUAUGACGCCCUGCACAUGCAG |
| | CAGAACCAGCTGTACAACGAGCTGAACCTG | GCCCUGCCUCCAAGA (SEQ ID NO: 355) |
| | GGGAGAAGAGAAGAGTACGACGTGCTGGAC | |
| | AAGCGGAGAGGCAGAGATCCTGAGATGGGC | |
| | GGCAAGCCCAGAAGAAAGAACCCTCAAGAG | |
| | GGCCTGTATAATGAGCTGCAGAAAGACAAG | |
| | ATGGCCGAGGCCTACAGCGAGATCGGAATG | |
| | AAGGGCGAGCGCAGAAGAGGCAAGGGACAC | |
| | GATGGACTGTACCAGGGCCTGAGCACAGCC | |
| | ACCAAGGACACATATGACGCCCTGCACATG | |
| | CAGGCCCTGCCTCCAAGA (SEQ ID NO: | |
| | 297) | |
| 2x Stop | TAATAG (No SEQ ID designated | UAAUAG (No SEQ ID designated for less |
| Codon | for less than 10 nucleotides) | than 10 nucleotides) |
| Upstream | aacaaacacaaac (SEQ ID NO: 373) | aacaaacacaaac (SEQ ID NO: 386) |
| miR122_2 | | |
| spacer | | |
| 1x miR122 | CAAACACCATTGTCACACTCCA (SEQ ID | CAAACACCAUUGUCACACUCCA (SEQ ID NO: |
| binding site | NO: 298) | 356) |
| Downstream | aacaacaaacaaaca (SEQ ID NO: | aacaacaaacaaaca (SEQ ID NO: 387) |
| miR122_2 | 374) | |
| Spacer | | |
| Internal | ACCACACAAATGGTCGCCGA (SEQ ID | ACCACACAAAUGGUCGCCGA (SEQ ID NO: 388) |
| Homology | NO: 375) | |
| Element | | |
| (Downstream) | | |
| 3′ exon | aactactgaaagcat (SEQ ID NO: | aacuacugaaagcau (SEQ ID NO: 389) |
| fragment⁷ | 376) | |
| 3′ intron | AAATAATTGTGCCTTTATACAGTAATGTAT | AAAUAAUUGUGCCUUUAUACAGUAAUGUAUaucgaAA |
| fragment⁷ | atcgaAAAATCCTCTAATTCAGGGAACACC | AAUCCUCUAAUUCAGGGAACACCUAAACAAacuaaga |
| | TAAACAAactaagaTGTAGGCAATCCTGAG | UGUAGGCAAUCCUGAGCUAAGCucuuaguACGCGUUC |
| | CTAAGCtcttagtACGCGTTCAGATAGAT | AGAUAGAU (SEQ ID NO: 390) |
| | (SEQ ID NO: 377) | |
| pA25 Spacer | AAAAAAAAAAAAAAAAAAAAAAAAA (SEQ | AAAAAAAAAAAAAAAAAAAAAAAAA (SEQ ID NO: |
| | ID NO: 295) | 366) |
| XbaI | ATCTAGAACG (SEQ ID NO: 378) | AU (No seq ID designated for less |
| Restriction | | than 4 bases) |
| Site | | |
| Polymerase | CTAGCATAACCCCTTGGGGCCTCTAAACGG | N/A |
| Terminator | GTCTTGAGGGGTTTTTTG (SEQ ID NO: | |
| Sequence | 379) | |

¹The exemplary DNA template component is an exemplary sense strand sequence from an exemplary double-stranded DNA template that can be used to make an RNA precursor.
²The exemplary RNA precursor sequence is an RNA sequence transcribed from the antisense strand of the exemplary double-stranded DNA template.
³The sequence in lowercase is an exemplary T7 RNA polymerase promoter (taatacgactcactata (SEQ ID NO: 417). The sequence following thet promoter is an exemplary transcription initiating region (GGGAGAAT (No SEQ ID designated for less than 10 nt)). The leading sequence preceding the promoter is an exemplary leading sequence (GGCCAGTGAATTG (SEQ ID NO: 418)) that supports template stability and engagement with the RNA polymerase.
⁴The 5′ UTR comprises Internal Homology Element (Upstream), 5′ UTR cloning site 1, Random Insert, 3X Frame Stop Codon and 5′ UTR cloning site 2.
⁵The 3′ UTR comprises a 2X Stop Codon, Upstream miR-122_2 spacer, 1x_miR-122 binding site, downstream miR-122_2 spacer, and Internal Homology Element (Downstream).
⁶The 5′ intron fragment and the 5′ exon fragment together make up the Upstream Intron sequence of Twort-ORF142 intron-V2 in Table 13A.
⁷The 3′ exon fragment and the 3′ intron fragment together make up the Downstream Intron sequence of Twort-ORF142 intron-V2 in Table 13A.

### Nanoparticle Delivery Vehicle

In some embodiments, the circRNA of the present disclosure is formulated in a nanoparticle for delivery. In particular, delivery is targeted by formulating a circRNA construct for potent and selective delivery to T cells. In some embodiments, the cargo for delivery is an anti-CD19 CAR. In some embodiments, the circRNA is encapsulated in a lipid nanoparticle (LNP). In some embodiments, the circRNA is encapsulated in a polymer-based nanoparticle. In some embodiments, the circRNA is encapsulated in a lipid-polymer based nanoparticle. In some embodiments, the circRNA is encapsulated in a virus-like particle (VLP). In some embodiments, the circRNA is encapsulated in engineered exosomes. In some embodiments, a targeting moiety is conjugated to the delivery vehicle to allow for targeted delivery, as disclosed herein.

In some embodiments, the circRNA is delivered without encapsulation in a nanoparticle. In some embodiments, the circRNA is delivered in complex with cationic peptides or polymers (i.e., ionizable lipids). In some embodiments, a targeting moiety is conjugated to a circRNA complex.

### Lipid Nanoparticles (LNPs)

In some embodiments, a circRNA molecule of the present disclosure is encapsulated in a lipid nanoparticle (LNP). LNP components are selected based on the target and cargo (e.g., circRNA molecules). LNP components that achieve optimal size, polydispersity index, percent encapsulation, and/or other desired features are selected for inclusion within the LNP to ensure targeted delivery. LNP components can include, for example, ionizable lipids, helper lipids, sterols, and/or polymer-lipids (e.g., PEG-lipids). The relative amounts, or molar ratios, of ionizable lipid, helper lipid, cholesterol, and polymer-lipid (e.g., PEG-lipid) are selected for a given target or administration route.

In general, LNPs are small solid or semi-solid particles possessing an exterior lipid layer with a hydrophilic exterior surface that is exposed to the non-LNP environment, an interior space which may aqueous (vesicle like) or non-aqueous (micelle like), and one or more hydrophobic inter-membrane space. LNP membranes may be lamellar or non-lamellar and may be comprised of 1, 2, 3, 4, 5 or more layers.

In some embodiments, the LNP comprises one or more polymer-lipids, an ionizable lipid, a phospholipid and a sterol lipid. In some embodiments, the LNP comprises one or more PEG-lipids, an ionizable lipid, a phospholipid and a sterol lipid. In some embodiments, the LNP comprises one or more polyethylene glycol (PEG) lipids, wherein the targeting moiety is conjugated to a conjugating PEG-lipid comprising a long chain fatty acid, or to cholesterol. In some embodiments, the LNP comprises two PEG-lipids, one of which is a conjugating PEG-lipid, and the thiol-maleimide conjugation is between the conjugating PEG-lipid and the conjugation handle.

In some embodiments, the LNP further comprises one or more ionizable lipids, one or more phospholipids, and one or more sterol lipids.

In some embodiments, the LNP comprises one or more ionizable lipids. In some embodiments, the LNP comprises one or more phospholipids. In some embodiments, the LNP comprises one or more sterol lipids. In some embodiments, the LNP comprises a combination of one or more polymer-lipids, ionizable lipids, phospholipids, and sterol lipids. In some embodiments, the LNP comprises a combination of one or more PEGs, ionizable lipids, phospholipids, and sterol lipids.

In some embodiments, the LNP comprises the one or more ionizable lipids at 30 to 49 mol % (e.g., at 30 to 45 mol %), the one or more phospholipids at 5 to 40 mol % (e.g., at 15 to 35 mol %), the one or more sterol lipids at 15 to 50 mol % (e.g., at 30 to 50 mol %), the PEG lipid (e.g., the DMG-PEG 2000) at 0 to 10 mol % (e.g., at 0 to 5 mol %), and the conjugating PEG-lipid (e.g., the DSPE-PEG 2000-maleimide) at 0.01-1 mol %.

In some embodiments, the LNP comprises the one or more ionizable lipids at 30 to 49 mol %, the one or more phospholipids at 5 to 40 mol %, the one or more sterol lipids at 15 to 50 mol %, the DMG-PEG 2000 lipid at 0 to 10 mol %, and the DSPE-PEG 2000-maleimide lipid at 0.01-1 mol %.

In some embodiments, the LNP comprises the one or more ionizable lipids at 30 to 45 mol %, the one or more phospholipids at 15 to 35 mol %, the one or more sterol lipids at 15 to 50 mol %, the DMG-PEG 2000 lipid at 0 to 5 mol %, and the DSPE-PEG 2000-maleimide lipid at 0.01-1 mol %.

In some embodiments, the LNP comprises the one or more ionizable lipids, the one or more phospholipids, the one or more sterol lipids, the DMG-PEG 2000 lipid, and the DSPE-PEG 2000-maleimide lipid at a ratio of 39:20:39:1.9:0.1 mol %.

In some embodiments, the LNP comprises the one or more ionizable lipids, the one or more phospholipids, the one or more sterol lipids, the DMG-PEG 2000 lipid, and the DSPE-PEG 2000-maleimide lipid combined at a molar ratio of 39:20:39:1.9:0.1.

In some embodiments, the LNP comprises an ionizable lipid, a phospholipid, a sterol lipid, a DMG-PEG 2000 lipid, and a DSPE-PEG 2000-maleimide lipid at a ratio of 39:20:39:1.9:0.1 mol %. In some embodiments, the LNP comprises ionizable lipid:DSPC:Cholesterol:DMG-PEG 2000:DSPE-PEG 2000-maleimide at a ratio of 39:20:39:1.9:0.1 mol %. In some embodiments, the LNP comprises ionizable lipid:DSPC:Cholesterol:DMG-PEG 2000:DSPE-polysarcosine at a ratio of 39:20:39:1.9:0.1 mol %.

In some embodiments, the LNP comprises ionizable lipid:DSPC:Cholesterol:DMG-PEG 2000:DSPE-PEG 2000-maleimide combined at a molar ratio of 39:20:39:1.9:0.1 mol %. In some embodiments, the LNP comprises ionizable lipid:DSPC:Cholesterol:DMG-PEG 2000:DSPE-polysarcosine combined at a ratio of 39:20:39:1.9:0.1 mol %.

### Polymer-Lipids

In some embodiments, the LNP comprises polymer-lipids, wherein the polymer includes but not limited to Polyethylene glycol (PEG), Poly(2-oxazoline) (POx), Poly(2-methyl-2-oxazoline) (PMOX), Poly(2-ethyl-2-oxazoline) (PEOX), Polysarcosine (PSar), Poly(carboxybetaine) (PCB), Poly(sulfobetaine) (PSB), Polyglycerol (PG), and Poly(vinylpyrrolidone) (PVP).

Polymer-lipids can reduce LNP aggregation, shield the LNPs from non-specific endocytosis, and reduce opsonization by serum proteins and reticuloendothelial clearance. In some embodiments, the LNP comprises one polyethylene glycol (PEG-lipid). In some embodiments, the LNP comprises one modified-PEG-lipid. In some embodiments, the LNP comprises two PEG-lipids. In some embodiments, the LNP comprises two or more PEG-lipids. In some embodiments, the LNP comprises two modified-PEG-lipids.

To facilitate conjugation, suitable chemical groups for modifying or functionalizing PEG-lipid include, but are not limited to, azide, trans-cyclooctene, phosphine, tetrazine, maleimide, N-hydroxysuccinimide (NHS) ester, imidoester, carbodiimide, haloacetyl, alkyne, pyridyl disulfide, hydrazide, or alkoxyamine. The chemically functionalized PEG-lipid then conjugates with the conjugation handle of targeting moiety. In some embodiments, the PEG is modified or functionalized with maleimide which conjugates with the thiol group of the cysteine in the conjugation handle GGGSCAAA (SEQ ID NO: 120) by thiol-maleimide conjugation.

PEG-lipids or modified-PEG-lipids may be alternately referred to as PEGylated lipids or PEG-lipids. Inclusion of a PEGylating lipid is used to enhance lipid nanoparticle structure, colloidal stability in vitro, and circulation time in vivo and a second PEG is used for conjugation. In some embodiments, the PEGylation is reversible in that the PEG moiety is gradually released in blood circulation.

In some embodiments, PEG is chemically modified by azide, trans-cyclooctene, phosphine, tetrazine, maleimide, N-hydroxysuccinimide (NHS) ester, imidoester, carbodiimide, haloacetyl, pyridyl disulfide, hydrazide, or alkoxyamine.

Exemplary PEG-lipids include but are not limited to modified-PEG phosphatidylethanolamines, modified-PEG phosphatidic acids, modified-PEG ceramides (PEG-CER), modified-PEG dialkylamines, modified-PEG diacylglycerols (PEG-DAG), Modified-PEG dialkylglycerols, and mixtures thereof may also be suitable. For example, in some embodiments, a PEG-lipid may be PEG-c-DOMG, PEG-DMG, PEG-DLPE, PEG-DMPE, PEG-DPPE, PEG-DSG or a PEG-DSPE lipid. In some embodiments, a PEG-lipid comprises DiMystyrlGlycerol (DMG), 1,2-Dipalmitoyl-rac-glycerol, methoxypolyethylene Glycol (DPG-PEG), or 1,2-Distearoyl-rac-glycero-3-methylpolyoxyethylene (DSG-PEG). In some embodiments, a PEG-lipid is DMG-PEG 2000, DPG-PEG 2000, or DSG-PEG 2000.

In some embodiments, the conjugating PEG-lipid comprises a long chain fatty acid. In some embodiments, the conjugating PEG-lipid comprises a lipid anchor component that is a long chain fatty acid, and the other PEG-lipid is C14. In some embodiments, the lipid anchor component comprises saturated or unsaturated alkyl chains having a length of C14-C22. In some embodiments, the lipid anchor component comprises saturated or unsaturated alkyl chains having a length of C14-C24. In some embodiments, the long chain fatty acid is greater than C14. In some embodiments, the long chain fatty acid is between C14 and C22. In some embodiments, the long chain fatty acid is between C14 and C24. In some embodiments, the long chain fatty acid is C16. In some embodiments, the long chain fatty acid is C18. In some embodiments, the long chain fatty acid is C20. In some embodiments, the long chain fatty acid is C22. In some embodiments, the long chain fatty acid is C24.

In some embodiments, the targeting moiety is conjugated to a conjugating PEG-lipid comprising a long chain fatty acid. In some embodiments, the targeting moiety is conjugated to a conjugating PEG-lipid comprising cholesterol.

In some embodiments, the LNP comprises a DSPE-PEG 2000 lipid and the targeting moiety is conjugated to the DSPE-PEG-2000 lipid. In some embodiments, the LNP comprises a DSPE-PEG 2000 lipid. In some embodiments, the targeting moiety is conjugated to the DSPE-PEG-2000 lipid. In some embodiments, the conjugating PEG-lipid is DSPE-PEG 2000-maleimide.

In some embodiments, the LNP comprises a DSPE-polysarcosine lipid, and wherein the targeting moiety is conjugated to the DSPE-polysarcosine lipid.

In some embodiments, the LNP comprises one or more PEG-lipids at 0-10 mol %. In some embodiments, the LNP comprises one or more PEG-lipids at 1-10 mol %. In some embodiments, the LNP comprises one or more PEG-lipids at 1 mol %. In some embodiments, the LNP comprises one or more PEG-lipids at 2 mol %. In some embodiments, the LNP comprises one or more PEG-lipids at 3 mol %. In some embodiments, the LNP comprises one or more PEG-lipids at 4 mol %. In some embodiments, the LNP comprises one or more PEG-lipids at 5 mol %. In some embodiments, the LNP comprises one or more PEG-lipids at 6 mol %. In some embodiments, the LNP comprises one or more PEG-lipids at 7 mol %. In some embodiments, the LNP comprises one or more PEG-lipids at 8 mol %. In some embodiments, the LNP comprises one or more PEG-lipids at 9 mol %. In some embodiments, the LNP comprises one or more PEG-lipids at 10 mol %.

In some embodiments, the LNP comprises a DMG-PEG 2000 lipid at 0-10 mol %. In some embodiments, the LNP comprises a DMG-PEG 2000 lipid at 1-10 mol %. In some embodiments, the LNP comprises a DMG-PEG 2000 lipid at 1 mol %. In some embodiments, the LNP comprises a DMG-PEG 2000 lipid at 2 mol %. In some embodiments, the LNP comprises a DMG-PEG 2000 lipid at 3 mol %. In some embodiments, the LNP comprises a DMG-PEG 2000 lipid at 4 mol %. In some embodiments, the LNP comprises a DMG-PEG 2000 lipid at 5 mol %. In some embodiments, the LNP comprises a DMG-PEG 2000 lipid at 6 mol %. In some embodiments, the LNP comprises a DMG-PEG 2000 lipid at 7 mol %. In some embodiments, the LNP comprises a DMG-PEG 2000 lipid at 8 mol %. In some embodiments, the LNP comprises a DMG-PEG 2000 lipid at 9 mol %. In some embodiments, the LNP comprises a DMG-PEG 2000 lipid at 10 mol %.

In some embodiments, the LNP comprises one or more conjugating PEG-lipids at 0.01-1 mol %.

In some embodiments, the LNP comprises a DSPE-PEG 2000-maleimide at 0.01-1 mol %.

In some embodiments, the LNP comprises two PEG-lipids at a total mol % between 0.01-6 mol %. In some embodiments, the LNP comprises two PEG-lipids at a total mol % of 0.01 mol %. In some embodiments, the LNP comprises two PEG-lipids at a total mol % of 0.02 mol %. In some embodiments, the LNP comprises two PEG-lipids at a total mol % of 0.03 mol %. In some embodiments, the LNP comprises two PEG-lipids at a total mol % of 0.04 mol %. In some embodiments, the LNP comprises two PEG-lipids at a total mol % of 0.05 mol %. In some embodiments, the LNP comprises two PEG-lipids at a total mol % of 0.06 mol %. In some embodiments, the LNP comprises two PEG-lipids at a total mol % of 0.07 mol %. In some embodiments, the LNP comprises two PEG-lipids at a total mol % of 0.08 mol %. In some embodiments, the LNP comprises two PEG-lipids at a total mol % of 0.09 mol %. In some embodiments, the LNP comprises two PEG-lipids at a total mol % of 0.1 mol %.

In some embodiments, the LNP comprises two PEG-lipids at a total mol % of 0.1 mol %. In some embodiments, the LNP comprises two PEG-lipids at a total mol % of 0.2 mol %. In some embodiments, the LNP comprises two PEG-lipids at a total mol % of 0.3 mol %. In some embodiments, the LNP comprises two PEG-lipids at a total mol % of 0.4 mol %. In some embodiments, the LNP comprises two PEG-lipids at a total mol % of 0.5 mol %. In some embodiments, the LNP comprises two PEG-lipids at a total mol % of 0.6 mol %. In some embodiments, the LNP comprises two PEG-lipids at a total mol % of 0.7 mol %. In some embodiments, the LNP comprises two PEG-lipids at a total mol % of 0.8 mol %. In some embodiments, the LNP comprises two PEG-lipids at a total mol % of 0.9 mol %.

In some embodiments, the LNP comprises two PEG-lipids at a total mol % of 1 mol %. In some embodiments, the LNP comprises two PEG-lipids at a total mol % of 1.5 mol %. In some embodiments, the LNP comprises two PEG-lipids at a total mol % of 2 mol %. In some embodiments, the LNP comprises two PEG-lipids at a total mol % of 2.5 mol %. In some embodiments, the LNP comprises two PEG-lipids at a total mol % of 3 mol %. In some embodiments, the LNP comprises two PEG-lipids at a total mol % of 3.5 mol %. In some embodiments, the LNP comprises two PEG-lipids at a total mol % of 4 mol %. In some embodiments, the LNP comprises two PEG-lipids at a total mol % of 4.5 mol %. In some embodiments, the LNP comprises two PEG-lipids at a total mol % of 5 mol %. In some embodiments, the LNP comprises two PEG-lipids at a total mol % of 5.5 mol %. In some embodiments, the LNP comprises two PEG-lipids at a total mol % of 6 mol %. In some embodiments, the LNP comprises two PEG-lipids at a total mol % of 6.5 mol %.

### Ionizable Lipids

In some embodiments, the LNP comprises an ionizable lipid. Ionizable lipids generally contain an amine-containing group on the head group. In some embodiments, the ionizable lipid comprises an ionizable cationic lipid. In some embodiments, the ionizable lipid comprises an ionizable anionic lipid.

In some embodiments, the ionizable lipid comprises a compound disclosed in WO 2024/019936 (Sago et al.), the entirety of which is incorporated by reference herein. In some embodiments, the ionizable lipid comprises a compound of Formula (IX-B-i) of WO 2024/019936 A1 (Sago et al.).

In some embodiments, the present disclosure provides a compound of Formula IX-B-i:

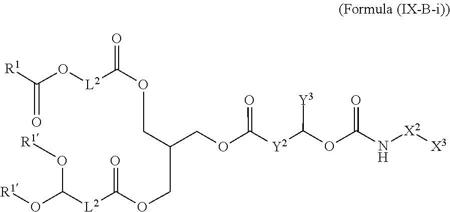

In some embodiments, R¹ in Formula (IX-B-i) is C₆₋₁₂ aliphatic. In some embodiments, R¹ is C₆₋₁₂ alkyl. In some embodiments, R¹ is C₆₋₁₂ alkenyl.

In some embodiments, R¹ is a bivalent saturated or unsaturated, straight or branched C₁₋₁₂ hydrocarbon chain. In some embodiments, R¹ is a bivalent saturated, straight or branched C₁₋₁₂ hydrocarbon chain. In some embodiments, R¹ is a bivalent unsaturated, straight or branched C₁₋₁₂ hydrocarbon chain. In some embodiments, R¹ is a bivalent saturated or unsaturated, straight or branched C₆₋₁₂ hydrocarbon chain. In some embodiments, R¹ is a bivalent saturated, straight or branched C₆₋₁₂ hydrocarbon chain. In some embodiments, R¹ is a bivalent unsaturated, straight or branched C₆₋₁₂ hydrocarbon chain.

In some embodiments, R^{1′} is independently C₆₋₁₂ aliphatic. In some embodiments, R^{1′} is independently C₆₋₁₂ alkyl. In some embodiments, R^{1′} is independently C₆₋₁₂ alkenyl. In some embodiments, R^{1′} is independently C₇₋₉ aliphatic. In some embodiments, R^{1′} is independently C₇₋₉ alkyl. In some embodiments, R^{1′} is independently C₇₋₉ alkenyl.

In some embodiments, L² in Formula (IX-B-i) is —(CH₂)—. In some embodiments, L² is —(CH₂)₂—. In some embodiments, L² is —(CH₂)₃—. In some embodiments, L² is —(CH₂)₄—. In some embodiments, L² is —(CH₂)₅—. In some embodiments, L² is —(CH₂)₆—. In some embodiments, L² is —(CH₂)₇—. In some embodiments, L² is —(CH₂)₈—.

In some embodiments, L²′ in Formula (IX-B-i) is —(CH₂)—. In some embodiments, L², is —(CH₂)₂—. In some embodiments, L²′ is —(CH₂)₃—. In some embodiments, L²′ is —(CH₂)₄—. In some embodiments, L²′ is —(CH₂)₅—. In some embodiments, L²′ is —(CH₂)₆—. In some embodiments, L²′ is —(CH₂)₇—. In some embodiments, L²′ is —(CH₂)₈—. In some embodiments, Y² in Formula (IX-B-i) is —(CH₂)—. In some embodiments, Y² is —(CH₂)₂—. In some embodiments, Y² is —(CH₂)₃—.

In some embodiments, Y³ in Formula (IX-B-i) is C₁₋₁₂ aliphatic. In some embodiments, Y³ is C₁-12 alkyl. In some embodiments, Y³ is C₄₋₈ aliphatic. In some embodiments, Y³ is C₄₋₈ alkyl. In some embodiments, Y³ is —CH₃. In some embodiments, Y³ is —CH₂CH₃. In some embodiments, Y³ is —(CH₂)₂CH₃. In some embodiments, Y³ is —(CH₂)₃CH₃. In some embodiments, Y³ is —(CH₂)₄CH₃. In some embodiments, Y³ is —(CH₂)₅CH₃. In some embodiments, Y³ is —(CH₂)₆CH₃. In some embodiments, Y³ is —(CH₂)₇CH₃.
In some embodiments, X²-X³ in Formula (IX-B-i) is

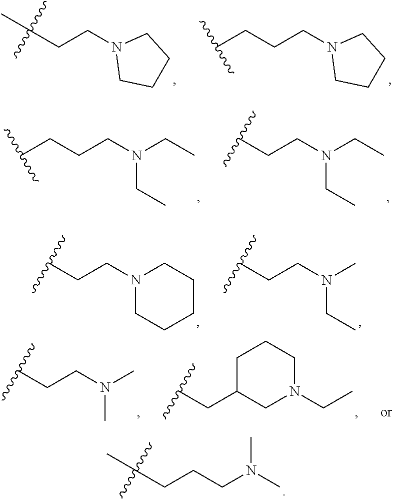

In some embodiments, the ionizable lipid is a compound selected from Table 14A.

TABLE 14A
Exemplary ionizable lipids of Formula (IX-B-i).

| Com- | |
|---|---|
| pound | Structure |
| A | 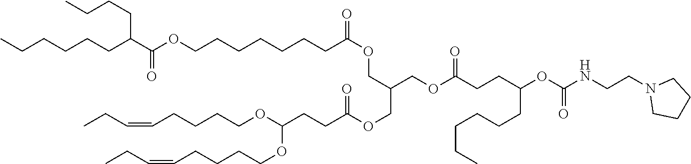 [SMILES: CC/C=C\CCCCOC(CCC(=O)OCC(COC(=O)CCCCCCCOC(=O)C(CCCC)CCCCCC)COC(=O)CCC(CCCCCC)OC(=O)NCCN1CCCC1)OCCCC/C=C\CC] |
| B | 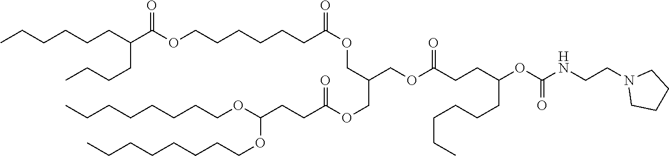 [SMILES: CCCCCCCCOC(CCC(=O)OCC(COC(=O)CCCCCCOC(=O)C(CCCC)CCCCCC)COC(=O)CCC(CCCCCC)OC(=O)NCCN1CCCC1)OCCCCCCCC] |
| C | 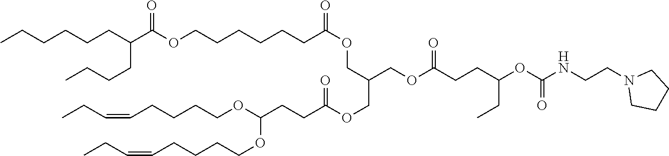 [SMILES: CC/C=C\CCCCOC(CCC(=O)OCC(COC(=O)CCCCCCOC(=O)C(CCCC)CCCCCC)COC(=O)CCC(CC)OC(=O)NCCN1CCCC1)OCCCC/C=C\CC] |
| D | 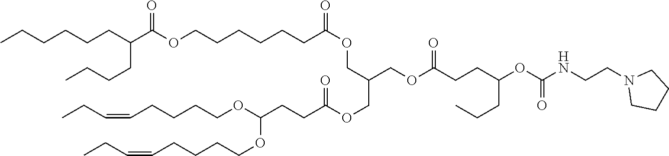 [SMILES: CC/C=C\CCCCOC(CCC(=O)OCC(COC(=O)CCCCCCOC(=O)C(CCCC)CCCCCC)COC(=O)CCC(CCC)OC(=O)NCCN1CCCC1)OCCCC/C=C\CC] |
| E | 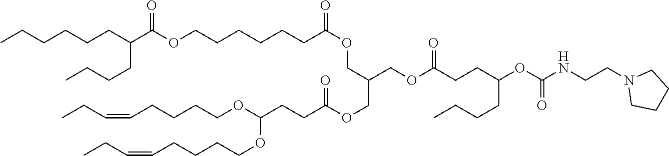 [SMILES: CC/C=C\CCCCOC(CCC(=O)OCC(COC(=O)CCCCCCOC(=O)C(CCCC)CCCCCC)COC(=O)CCC(CCCC)OC(=O)NCCN1CCCC1)OCCCC/C=C\CC] |
| F | 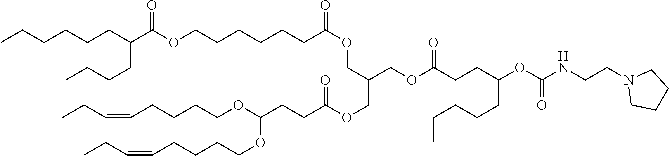 [SMILES: CC/C=C\CCCCOC(CCC(=O)OCC(COC(=O)CCCCCCOC(=O)C(CCCC)CCCCCC)COC(=O)CCC(CCCCC)OC(=O)NCCN1CCCC1)OCCCC/C=C\CC] |
| G | 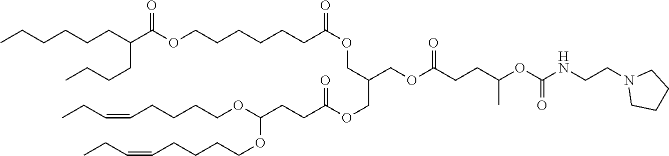 [SMILES: CC/C=C\CCCCOC(CCC(=O)OCC(COC(=O)CCCCCCOC(=O)C(CCCC)CCCCCC)COC(=O)CCC(C)OC(=O)NCCN1CCCC1)OCCCC/C=C\CC] |
| H | 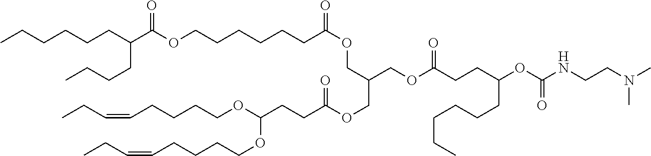 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN(C)C)OCCCC/C=C\CC] |
| I | 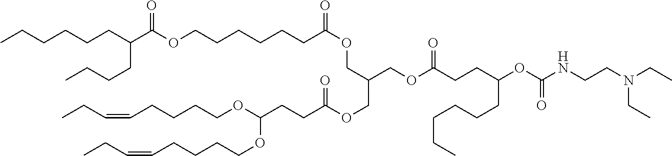 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN(CC)CC)OCCCC/C=C\CC] |
| J | 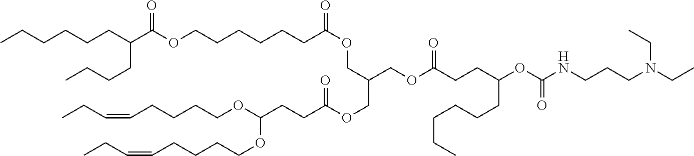 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCCN(CC)CC)OCCCC/C=C\CC] |
| K | 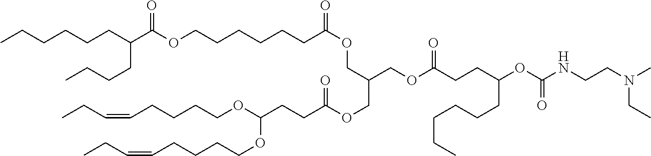 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN(C)CC)OCCCC/C=C\CC] |
| L | 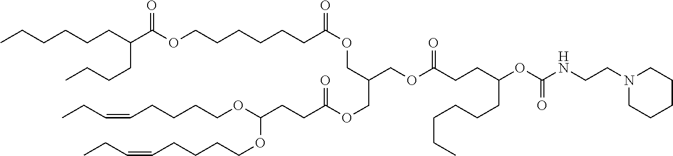 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN1CCCCC1)OCCCC/C=C\CC] |
| M | 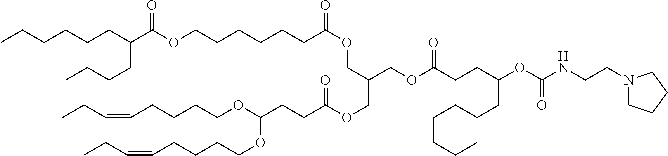 [SMILES: CC/C=C\CCCCOC(CCC(=O)OCC(COC(=O)CCCCCCOC(=O)C(CCCC)CCCCCC)COC(=O)CCC(CCCCCCC)OC(=O)NCCN1CCCC1)OCCCC/C=C\CC] |
| N | 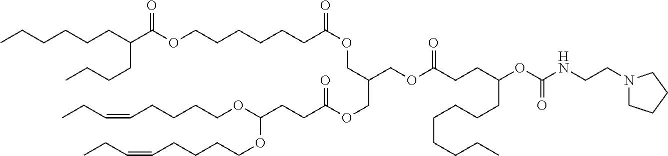 [SMILES: CC/C=C\CCCCOC(CCC(=O)OCC(COC(=O)CCCCCCOC(=O)C(CCCC)CCCCCC)COC(=O)CCC(CCCCCCCC)OC(=O)NCCN1CCCC1)OCCCC/C=C\CC] |

In some embodiments, the ionizable lipid comprises a compound disclosed in WO 2021/141969A1 (Hamilton et al.), the entirety of which is incorporated by reference herein. In some embodiments, the ionizable lipid comprises a compound of Formula (I) of WO 2021/141969 A1 (Hamilton et al.).

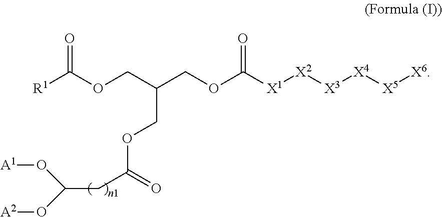

In some embodiments, R¹ in Formula (I) comprises C₉-C₂₀ alkyl or C₉-C₂₀ alkenyl with 1-3 units of unsaturation. For example, in some embodiments R¹ comprises a C₉-C₂₀ alkenyl with 2 units of unsaturation, such as, without limitation, a C₁₇ alkenyl with 2 units of unsaturation.

In some embodiments, X³, X⁵, and X⁶ in Formula (I) are independently absent.

In some embodiments, X¹ is —O—. In some embodiments, X¹ is absent.

In some embodiments, X² is

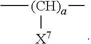

In some embodiments, X² is —(CH₂)ₐ— or —CH(OH)—. In some embodiments, a is an integer between 0 and 6. In some embodiments, a is 0, 1, 2, 3, 4, 5, or 6. In some embodiments, a is 0 and X² is absent. In some embodiments, a is 1.

In some embodiments, X⁷ is independently hydrogen or hydroxyl. In some embodiments, X⁷ is hydroxyl. In some embodiments, X⁷ is hydrogen.

In some embodiments, X⁴ is a 6-membered heterocyclyl optionally substituted with 1 or 2 C₁-C₆ alkyl groups. In some embodiments, the heterocyclyl comprises at least one nitrogen. For example, some embodiments, X⁴ is piperidinyl. In some embodiments, X⁴ is ethylpiperidinyl.

In some embodiments, A¹ and A² are independently C₅-C₁₂ alkyl or C₅-C₁₂ alkenyl with 1-3 units of unsaturation. In some embodiments, A¹ and A² are independently C₅-C₁₂ alkenyl with 1 unit of unsaturation. In some embodiments, A¹ is C₈ alkenyl with 1 unit of unsaturation. In some embodiments, A² is C₈ alkenyl with 1 unit of unsaturation.

In some embodiments, n1 is an integer between 1 and 6. In some embodiments, n1 is 1, 2, 3, 4, 5, or 6. In some embodiments, n1 is 2.

In some embodiments, the ionizable lipid comprises a compound disclosed in WO 2022/140252 A1 (Patwardhan et al.), the entirety of which is incorporated by reference herein. In some embodiments, the ionizable lipid comprises a compound of Formula (III-a-i) of WO 2022/140252 A1 (Patwardhan et al.), or its N-oxide:

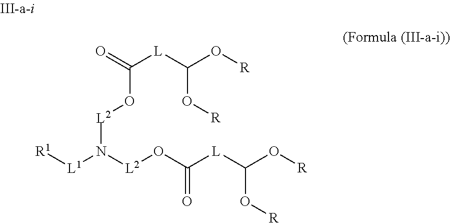

In some embodiments, R¹ is hydrogen.

In some embodiments, L¹ is C₂-C₆ heteroalkylenyl comprising at least 1 heteroatom. In some embodiments, the heteroatom is oxygen. For example, in some embodiments, L¹ is a C₄ heteroalkylenyl comprising 1 oxygen atom, such as, for example and without limitation, —OCH₂CH₂CH₂—. In some embodiments, L¹ is a C3 heteroalkylenyl comprising 1 oxygen atom, such as, for example and without limitation, —OCH₂CH₂—.

In some embodiments, each R is independently C₆-C₁₂ alkyl or C₆-C₁₂ alkenyl with 1-3 units of unsaturation.

In some embodiments, each L is independently C₁-C₅ alkylenyl.

In some embodiments, each L² is independently C₄-C₈ alkylenyl.

In some embodiments, the LNP comprises one or more ionizable lipids at about 30-49 mole percent (mol %). In some embodiments, the LNP comprises one or more ionizable lipids at about 30 mol %. In some embodiments, the LNP comprises one or more ionizable lipids at about 31 mol %. In some embodiments, the LNP comprises one or more ionizable lipids at about 32 mol %. In some embodiments, the LNP comprises one or more ionizable lipids at about 33 mol %. In some embodiments, the LNP comprises one or more ionizable lipids at about 34 mol %. In some embodiments, the LNP comprises one or more ionizable lipids at about 35 mol %. In some embodiments, the LNP comprises one or more ionizable lipids at about 36 mol %. In some embodiments, the LNP comprises one or more ionizable lipids at about 37 mol %. In some embodiments, the LNP comprises one or more ionizable lipids at about 38 mol %. In some embodiments, the LNP comprises one or more ionizable lipids at about 39 mol %. In some embodiments, the LNP comprises one or more ionizable lipids at about 40 mol %. In some embodiments, the LNP comprises one or more ionizable lipids at about 41 mol %. In some embodiments, the LNP comprises one or more ionizable lipids at about 42 mol %. In some embodiments, the LNP comprises one or more ionizable lipids at about 43 mol %. In some embodiments, the LNP comprises one or more ionizable lipids at about 44 mol %. In some embodiments, the LNP comprises one or more ionizable lipids at about 45 mol %. In some embodiments, the LNP comprises one or more ionizable lipids at about 46 mol %. In some embodiments, the LNP comprises one or more ionizable lipids at about 47 mol %. In some embodiments, the LNP comprises one or more ionizable lipids at about 48 mol %. In some embodiments, the LNP comprises one or more ionizable lipids at about 49 mol %. In some embodiments, the LNP comprises one or more ionizable lipids at less than about 50 mol %.

In some embodiments, the ionizable lipid is selected from Table 14B.

TABLE 14B
Exemplary Structures of Ionizable Lipids

| Ionizable | |
|---|---|
| Lipid | Structure |
| Com- pound 1 | 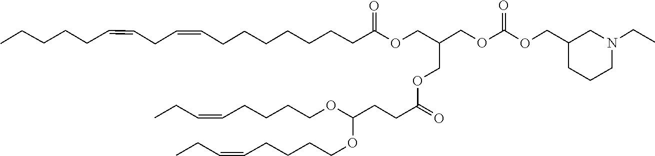 [SMILES: CC/C=C\CCCCOC(CCC(=O)OCC(COC(=O)CCCCCCC/C=C\C/C=C\CCCCC)COC(=O)OCC1CCCN(CC)C1)OCCCC/C=C\CC] |
| Com- pound 2 | 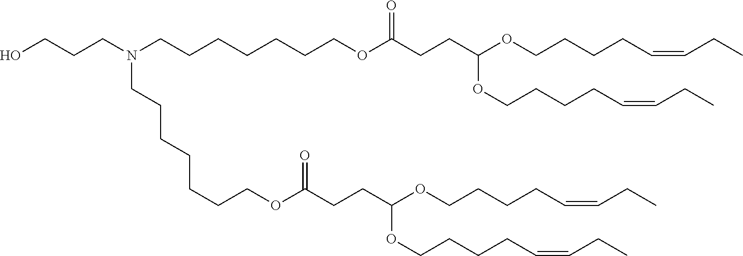 [SMILES: CC/C=C\CCCCOC(CCC(=O)OCCCCCCCN(CCCO)CCCCCCCOC(=O)CCC(OCCCC/C=C\CC)OCCCC/C=C\CC)OCCCC/C=C\CC] |
| Com- pound 3 | 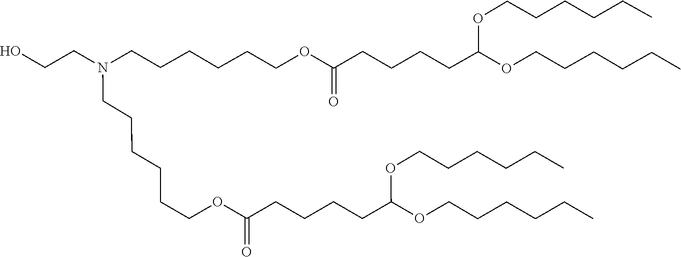 [SMILES: CCCCCCOC(CCCCC(=O)OCCCCCCN(CCO)CCCCCCOC(=O)CCCCC(OCCCCCC)OCCCCCC)OCCCCCC] |
| Com- pound 4 | 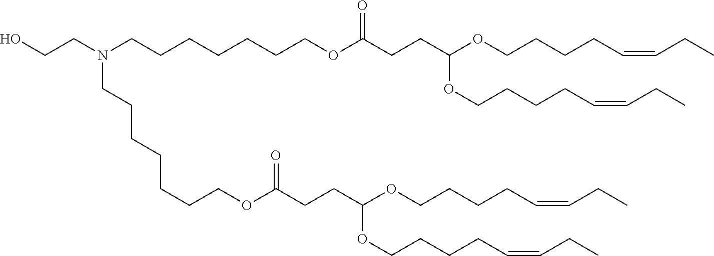 [SMILES: CC/C=C\CCCCOC(CCC(=O)OCCCCCCCN(CCO)CCCCCCCOC(=O)CCC(OCCCC/C=C\CC)OCCCC/C=C\CC)OCCCC/C=C\CC] |
| Com- pound 5 | 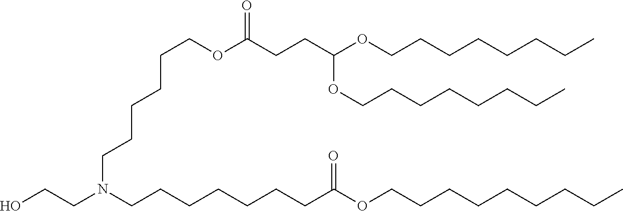 [SMILES: CCCCCCCCCOC(=O)CCCCCCCN(CCO)CCCCCCOC(=O)CCC(OCCCCCCCC)OCCCCCCCC] |
| Com- pound 6 | 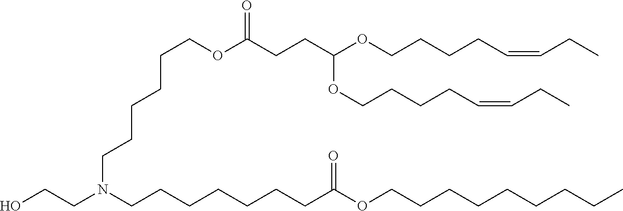 [SMILES: CC/C=C\CCCCOC(CCC(=O)OCCCCCCN(CCO)CCCCCCCC(=O)OCCCCCCCCC)OCCCC/C=C\CC] |
| Com- pound 7 | 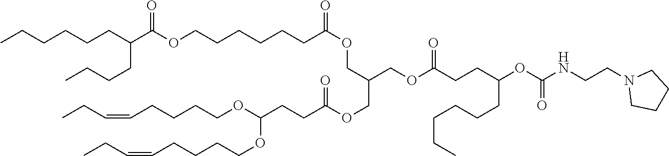 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN1CCCC1)OCCCC/C=C\CC] |
| Com- pound 8 | 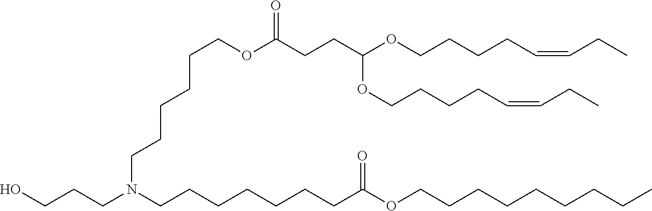 [SMILES: CC/C=C\CCCCOC(CCC(=O)OCCCCCCN(CCCO)CCCCCCCC(=O)OCCCCCCCCC)OCCCC/C=C\CC] |

In some embodiments, the ionizable lipid is:

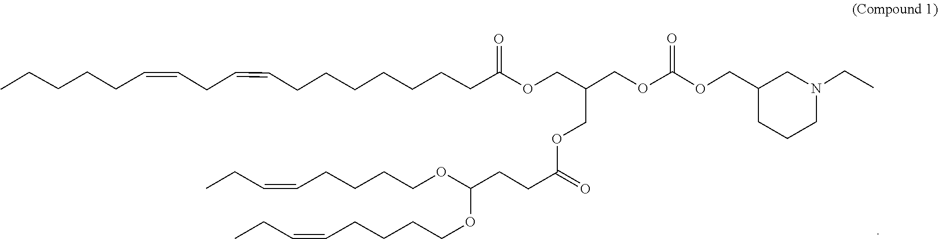 [SMILES: CC/C=C\CCCCOC(CCC(=O)OCC(COC(=O)CCCCCCC/C=C\C/C=C\CCCCC)COC(=O)OCC1CCCN(CC)C1)OCCCC/C=C\CC]

In some embodiments, the ionizable lipid is:

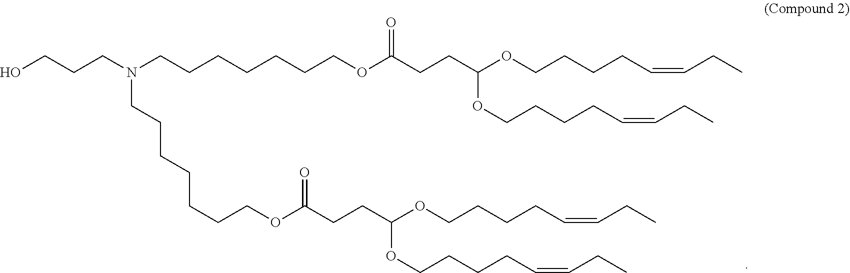 [SMILES: CC/C=C\CCCCOC(CCC(=O)OCCCCCCCN(CCCO)CCCCCCCOC(=O)CCC(OCCCC/C=C\CC)OCCCC/C=C\CC)OCCCC/C=C\CC]

In some embodiments, the ionizable lipid is Compound 3:

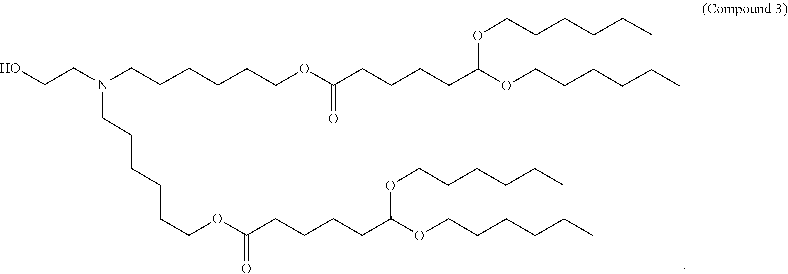 [SMILES: CCCCCCOC(CCCCC(=O)OCCCCCCN(CCO)CCCCCCOC(=O)CCCCC(OCCCCCC)OCCCCCC)OCCCCCC]

In some embodiments, the ionizable lipid is:

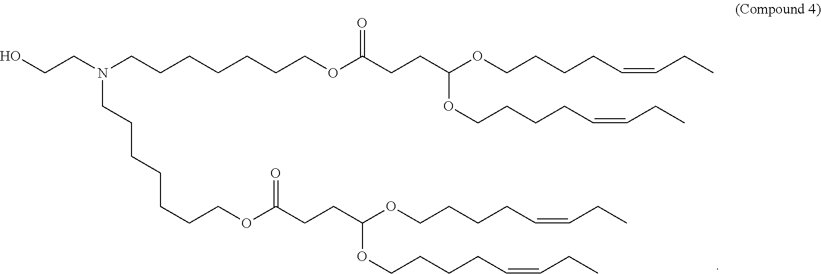 [SMILES: CC/C=C\CCCCOC(CCC(=O)OCCCCCCCN(CCO)CCCCCCCOC(=O)CCC(OCCCC/C=C\CC)OCCCC/C=C\CC)OCCCC/C=C\CC]

In some embodiments, the ionizable lipid is:

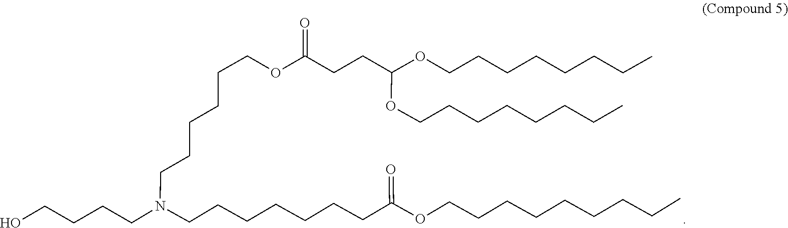 [SMILES: CCCCCCCCCOC(=O)CCCCCCCN(CCCCO)CCCCCCOC(=O)CCC(OCCCCCCCC)OCCCCCCCC]

In some embodiments, the ionizable lipid is:

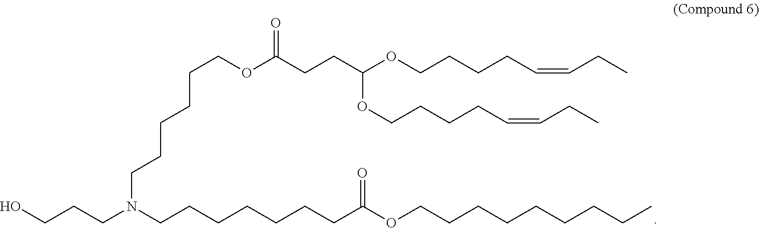 [SMILES: CC/C=C\CCCCOC(CCC(=O)OCCCCCCN(CCCO)CCCCCCCC(=O)OCCCCCCCCC)OCCCC/C=C\CC]

In some embodiments, the ionizable lipid is:

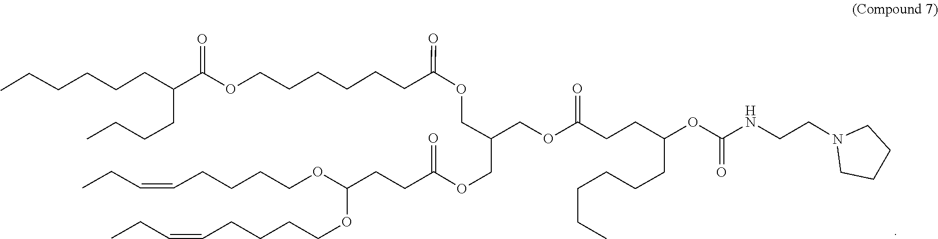 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN1CCCC1)OCCCC/C=C\CC]

In some embodiments, the ionizable lipid is:

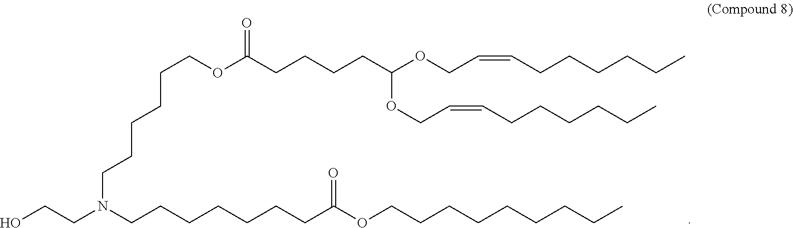 [SMILES: CCCCCC/C=C\COC(CCCCC(=O)OCCCCCCN(CCO)CCCCCCCC(=O)OCCCCCCCCC)OC/C=C\CCCCCC]

### Phospholipids

In some embodiments, one or more phospholipids include a phospholipid moiety. In some embodiments, the LNP comprises one or more phospholipids at about 5-40 mol %. In some embodiments, the LNP comprises one or more phospholipids at about 5 mol %. In some embodiments, the LNP comprises one or more phospholipids at about 10 mol %. In some embodiments, the LNP comprises one or more phospholipids at about 15 mol %. In some embodiments, the LNP comprises one or more phospholipids at about 20 mol %. In some embodiments, the LNP comprises one or more phospholipids at about 25 mol %. In some embodiments, the LNP comprises one or more phospholipids at about 30 mol %. In some embodiments, the LNP comprises one or more phospholipids at about 35 mol %. In some embodiments, the LNP comprises one or more phospholipids at about 40 mol %.

In some embodiments, one or more phospholipids include one or more fatty acid moieties. In some embodiments, one or more phospholipids include a phospholipid moiety and one or more fatty acid moieties. In some embodiments, a phospholipid moiety includes but is not limited to phosphatidyl choline, phosphatidyl ethanolamine, phosphatidyl glycerol, phosphatidyl serine, phosphatidic acid, 2-lysophosphatidyl choline, and sphingomyelin. In some embodiments, a fatty acid moiety includes but is not limited to lauric acid, myristic acid, myristoleic acid, palmitic acid, palmitoleic acid, stearic acid, oleic acid, linoleic acid, alphalinolenic acid, erucic acid, phytanic acid, arachidic acid, arachidonic acid, eicosapentaenoic acid, behenic acid, docosapentaenoic acid, and docosahexaenoic acid.

Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Exemplary phospholipids include but are not limited to 1,2-distearoyl-snglycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycerophosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycerophosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoy 1-sn-glycero-3-phosphocholine (OChemsPC), 1-hexadecyl snglycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2 oleoylphosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or combinations thereof. In some embodiments, the one or more phospholipids is 1,2-distearoyl-snglycero-3-phosphocholine (DSPC). In some embodiments, the one or more phospholipids is DMPC. In some embodiments, the phospholipid is DSPC. In some embodiments, the phospholipid is DMPC.

In some embodiments, the one or more phospholipids comprises 1,2-dioleoyl-sn-glycero-3-phosphoethanolamine-N-(succinyl) (succinyl PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), 1,2-dioleoyl-sn-glycero-3-phosphoethanolamine (DOPE), 1,2-dimyristoyl-sn-glycero-3-phosphocholine (DMPC), 1,2-dipalmitoyl-sn-glycero-3-phosphocholine (DPPC), or a combination thereof.

### Sterol Lipids

In some embodiments, the LNP comprises one or more sterol lipids at about 15 mol % to about 49 mol %. In some embodiments, the LNP comprises one or more sterol lipids at about 15 mol %. In some embodiments, the LNP comprises one or more sterol lipids at about 16 mol %. In some embodiments, the LNP comprises one or more sterol lipids at about 17 mol %. In some embodiments, the LNP comprises one or more sterol lipids at about 18 mol %. In some embodiments, the LNP comprises one or more sterol lipids at about 19 mol %. In some embodiments, the LNP comprises one or more sterol lipids at about 20 mol %. In some embodiments, the LNP comprises one or more sterol lipids at about 21 mol %. In some embodiments, the LNP comprises one or more sterol lipids at about 22 mol %. In some embodiments, the LNP comprises one or more sterol lipids at about 23 mol %. In some embodiments, the LNP comprises one or more sterol lipids at about 24 mol %. In some embodiments, the LNP comprises one or more sterol lipids at about 25 mol %. In some embodiments, the LNP comprises one or more sterol lipids at about 26 mol %. In some embodiments, the LNP comprises one or more sterol lipids at about 27 mol %. In some embodiments, the LNP comprises one or more sterol lipids at about 28 mol %. In some embodiments, the LNP comprises one or more sterol lipids at about 29 mol %. In some embodiments, the LNP comprises one or more sterol lipids at about 30 mol %. In some embodiments, the LNP comprises one or more sterol lipids at about 31 mol %. In some embodiments, the LNP comprises one or more sterol lipids at about 32 mol %. In some embodiments, the LNP comprises one or more sterol lipids at about 33 mol %. In some embodiments, the LNP comprises one or more sterol lipids at about 34 mol %.

In some embodiments, the LNP comprises one or more sterol lipids at about 35 mol %. In some embodiments, the LNP comprises one or more sterol lipids at about 36 mol %. In some embodiments, the LNP comprises one or more sterol lipids at about 37 mol %. In some embodiments, the LNP comprises one or more sterol lipids at about 38 mol %. In some embodiments, the LNP comprises one or more sterol lipids at about 39 mol %. In some embodiments, the LNP comprises one or more sterol lipids at about 40 mol %.

In some embodiments, the LNP comprises one or more sterol lipids at about 41 mol %. In some embodiments, the LNP comprises one or more sterol lipids at about 42 mol %. In some embodiments, the LNP comprises one or more sterol lipids at about 43 mol %. In some embodiments, the LNP comprises one or more sterol lipids at about 44 mol %. In some embodiments, the LNP comprises one or more sterol lipids at about 45 mol %. In some embodiments, the LNP comprises one or more sterol lipids at about 46 mol %. In some embodiments, the LNP comprises one or more sterol lipids at about 47 mol %. In some embodiments, the LNP comprises one or more sterol lipids at about 48 mol %. In some embodiments, the LNP comprises one or more sterol lipids at about 49 mol %.

In some embodiments, a sterol lipid is a cholesterol, or a variant or derivative thereof. In some embodiments, the one or more sterol lipids is cholesterol.

In some embodiments, the sterol lipid is cholesterol.

In some embodiments, a cholesterol is modified. In some embodiments, a cholesterol is an oxidized cholesterol. In some embodiments, a cholesterol is esterified cholesterol. Unmodified cholesterol can be acted upon by enzymes to form variants that are side-chain or ring oxidized. In some embodiments, a cholesterol can be oxidized on the beta-ring structure or on the hydrocarbon tail structure. In some embodiments, a sterol is a phytosterol. Exemplary sterols include but are not limited to 25-hydroxycholesterol (25-OH), 20α-hydroxycholesterol (20α-OH), 27-hydroxycholesterol, 6-keto-5α-hydroxycholesterol, 7-ketocholesterol, 7β-hydroxycholesterol, 7α-hydroxycholesterol, 7β-25-dihydroxycholesterol, beta-sitosterol, stigmasterol, brassicasterol, campesterol, or combinations thereof. In some embodiments, a side-chain oxidized cholesterol can enhance cargo delivery relative to other cholesterol variants. In some embodiments, a cholesterol is an unmodified cholesterol.

In some embodiments, the LNP comprises cholesterol at about 20-40 mol %. In some embodiments, the LNP comprises cholesterol at about 20 mol %. In some embodiments, the LNP comprises cholesterol at about 25 mol %. In some embodiments, the LNP comprises cholesterol at about 30 mol %.

In some embodiments, the LNP comprises cholesterol at about 35 mol %. In some embodiments, the LNP comprises cholesterol at about 36 mol %. In some embodiments, the LNP comprises cholesterol at about 37 mol %. In some embodiments, the LNP comprises cholesterol at about 38 mol %. In some embodiments, the LNP comprises cholesterol at about 39 mol %. In some embodiments, the LNP comprises cholesterol at about 40 mol %.

In some embodiments, the LNP comprises the exemplary compositions listed in Table 16.

TABLE 16
Exemplary LNP Compositions

| | LNP Composition | | | | | |
|---|---|---|---|---|---|---|
| | | | | | % DSPE- | |
| | % ionizable | | % | % DMG- | PEG2000- | % DSG- |
| LNP | lipid | % DSPC | cholesterol | PEG2000 | Maleimide | PEG2000 |
| LNP-1 | 47.5% | 10% | 40.5% | 2% | 0% | 0% |
| (“Base | | | | | | |
| LNP”) | | | | | | |
| LNP-2 | 47.5% | 10% | 40.5% | 1.5% | 0.5% | 0% |
| (“Reference | | | | | | |
| formulation”) | | | | | | |
| LNP-3 | 60% | 8% | 30% | 1.5% | 0.5% | 0% |
| LNP-4 | 46% | 15% | 37% | 1.5% | 0.5% | 0% |
| LNP-5 | 45% | 5% | 48% | 1.5% | 0.5% | 0% |
| LNP-6 | 41% | 13% | 44% | 1.5% | 0.5% | 0% |
| LNP-7 | 31% | 18% | 49% | 1.5% | 0.5% | 0% |
| LNP-8 | 26% | 26% | 46% | 1.5% | 0.5% | 0% |
| LNP-9 | 33% | 23% | 42% | 1.5% | 0.5% | 0% |
| LNP-10 | 25% | 33% | 40% | 1.5% | 0.5% | 0% |
| LNP-11 | 39% | 20% | 39% | 1.5% | 0.5% | 0% |
| LNP-12 | 47% | 25% | 26% | 1.5% | 0.5% | 0% |
| LNP-13 | 40.5% | 29.6% | 27.9% | 1.5% | 0.5% | 0% |
| LNP-14 | 33% | 34% | 31% | 1.5% | 0.5% | 0% |
| LNP-15 | 43% | 23% | 32% | 1.5% | 0.5% | 0% |
| LNP-16 | 28% | 39% | 31% | 1.5% | 0.5% | 0% |
| LNP-17 | 35% | 28% | 35% | 1.5% | 0.5% | 0% |
| LNP-18 | 39% | 20% | 39% | 1.9% | 0.1% | 0% |
| LNP-19 | 47.5% | 10% | 40.5% | 1.9% | 0.1% | 0% |
| LNP-20 | 47.5% | 10% | 40.5% | 1.5% | 0.1% | 0.4% |
| LNP-21 | 47.5% | 10% | 40.5% | 1.0% | 0.1% | 0.9% |
| LNP-22 | 39% | 20% | 39% | 2.0% | 0% | 0% |
| (“non- | | | | | | |
| targeted”) | | | | | | |

### Viral Particles

In some embodiments, the circular RNA of the present disclosure is encapsulated in a viral particle. In some embodiments, the circular RNA is encapsulated in an engineered viral particle. Viral particles include recombinant viruses and virus-like particles (VLPs). In some embodiments, the circular RNA is delivered using VLPs.

As used herein, the term “Virus-like particles” (VLPs) are molecules that closely resemble viruses but are non-infectious. They can be naturally occurring or synthesized through the individual expression of viral structural proteins, which can then self-assemble into the virus-like structure. Combinations of structural capsid proteins from different viruses can be used to create recombinant VLPs. VLPs can be produced from different viruses, such as adeno-associated viruses, retroviruses, lentiviruses and vesiculoviruses. VLPs can be produced in multiple cell culture systems including bacteria, mammalian cell lines, insect cell lines, yeast and plant cells.

### Other Delivery Vehicles

In some embodiments, other delivery vehicles are used to deliver the circular RNAs described herein. The vehicles may include other lipid-based particles such as lipidoids, liposomes, lipoplexes, micelles, multilamellar vesicle (MLV), unilamellar vesicle (SUV), polymer-based nanoparticles, hybrid polymer-lipid nanoparticles, protein-based nanoparticles, nanoemulsion, and exosomes. In some embodiments, the compositions may be encapsulated into a delivery agent described herein and/or known in the art for targeted delivery.

In some embodiments, the circRNA molecules discussed herein may be delivered using engineered exosomes. Exosomes are tiny vesicles smaller than 50 nm secreted by mature reticulocytes, which are associated with transferrin receptors and function in antigen presentation during the regulation of immune cells. In some embodiments, exosomes improve bioavailability of some drugs when taken orally, reducing the total dose required for administration, and minimizing side effects.

In some embodiments, circular RNA may also be constructed or altered such that their properties are suitable for different administration routes, such as parenteral (intravenously, intramuscularly or subcutaneously), oral, rectal, ophthalmic and/or topical administration. In some embodiments, the circular RNA of the present disclosure is administered intravenously.

In some embodiments, circular RNA of the present disclosure can be formulated for controlled release. As used herein, “controlled release” refers to a pharmaceutical composition or compound release profile that conforms to a particular pattern of release to affect a therapeutic outcome. As used herein, “sustained release” refers to a pharmaceutical composition or compound that conforms to a release rate over a specific period of time. The period of time may include, but is not limited to, hours, days, weeks, months and years. In some embodiments, the compositions may be encapsulated into a delivery agent described herein and/or known in the art for controlled release.

### Methods of Treatment

In some aspects, the circular RNAs, nucleic acid molecules, and compositions thereof of the present disclosure are used for therapy of various diseases.

In some embodiments, the circular RNAs, nucleic acid molecules, and compositions thereof are used for treating a disease, e.g., an autoimmune disease or a cancer, in a human subject. In some embodiments, the present disclosure provides a method for treating or preventing a disease using the circular RNAs, nucleic acid molecules, and compositions thereof described herein.

In some embodiments, the circRNAs encoding a chimeric antigen receptor (CAR), and compositions thereof, are used to treat B cell-mediated diseases, including B cell-mediated autoimmune diseases. In some embodiments, the circRNAs encoding an anti-CD19 CAR, and compositions thereof, are used to treat B cell-mediated diseases, including B cell-mediated autoimmune diseases.

As a non-limiting example, the circular RNAs and compositions thereof are used to target immune cells. In some embodiments, circular RNAs and compositions thereof are administered to a subject. In some embodiments, the administration is intravenous. In some embodiments, the subject is a human subject.

In some aspects, provided herein is a method of depleting B cells comprising administering to a subject in need thereof the circRNA therapeutic of the present disclosure.

In some embodiments, provided herein is a method of depleting B cells, comprising administering to a subject in need thereof an effective amount of the composition of the present disclosure, wherein the administration results in depletion of B cells relative to pre-treatment.

In some embodiments, provided herein is a composition of the present disclosure for use in the depletion of B cells.

In some embodiments, provided herein is use of a composition of the present disclosure for the depletion of B cells.

In some embodiments, provided herein is use of a composition of the present disclosure for the manufacture of a medicament for depletion of B cells.

In some embodiments, provided herein is a method of treating a B cell-mediated disease comprising administering to a subject in need thereof the circRNA therapeutic of the present disclosure.

In some embodiments, provided herein is a circRNA therapeutic of the present disclosure for use in the treatment of a B cell-mediated disease.

In some embodiments, provided herein is use of a circRNA therapeutic of the present disclosure for the treatment of a B cell-mediated disease.

In some embodiments, provided herein is use of a circRNA therapeutic of the present disclosure for the manufacture of a medicament for treating a B cell-mediated disease.

In some embodiments, provided herein is a method of treating a B cell-mediated disease comprising administering to a subject in need thereof an effective amount of the composition of the present disclosure.

In some embodiments, provided herein is a composition of the present disclosure for use in the treatment of a B cell-mediated disease.

In some embodiments, provided herein is use of a composition of the present disclosure for the treatment of a B cell-mediated disease.

In some embodiments, provided herein is use of a composition of the present disclosure for the manufacture of a medicament for treating a B cell-mediated disease.

In some embodiments, provided herein is a method of treating an autoimmune disease comprising administering to a subject in need thereof an effective amount of the composition of the present disclosure.

In some embodiments, provided herein is a composition of the present disclosure for use in the treatment of an autoimmune disease.

In some embodiments, provided herein is use of a composition of the present disclosure for the treatment of an autoimmune disease.

In some embodiments, provided herein is use of a composition of the present disclosure for the manufacture of a medicament for treating an autoimmune disease.

In some embodiments, provided herein is a method of treating a cancer comprising administering to a subject in need thereof an effective amount of the composition of the present disclosure.

In some embodiments, provided herein is a composition of the present disclosure for use in the treatment of a cancer.

In some embodiments, provided herein is use of a composition of the present disclosure for the treatment of a cancer.

In some embodiments, provided herein is use of a composition of the present disclosure for the manufacture of a medicament for treating a cancer.

### Autoimmune Disease

In some embodiments, the circRNA or circRNA therapeutics described herein are useful for the treatment of B-cell mediated autoimmune diseases characterized by pathogenic B-cell activity, such as, e.g., autoimmune diseases of the central and peripheral nervous systems, autoimmune vasculitides, autoimmune renal diseases, systemic autoimmune and connective tissue disorders, autoimmune endocrine and hepatopancreatobiliary diseases, autoimmune gastrointestinal diseases, autoimmune hematologic diseases, autoimmune dermatologic diseases, and interstitial lung disease associated with autoimmune, rheumatologic, or connective tissue diseases. Such autoimmune diseases include, e.g., autoimmune encephalitis, including, e.g., antibody-mediated autoimmune encephalitis and paraneoplastic autoimmune encephalitis; acute disseminated encephalomyelitis (ADEM); autoimmune demyelinating diseases of the central nervous system, including, e.g., multiple sclerosis (including, e.g., relapsing multiple sclerosis (RMS), relapsing-remitting multiple sclerosis (RRMS), active secondary progressive multiple sclerosis (SPMS), and primary progressive multiple sclerosis (PPMS)), neuromyelitis optica spectrum disorder (NMOSD), myelin oligodendrocyte glycoprotein antibody-associated disease (MOGAD), stiff person syndrome (SPS), and myasthenia gravis (MG); autoimmune demyelinating diseases of the peripheral nervous system, including, e.g., chronic inflammatory demyelinating polyradiculoneuropathy (CIDP), autoimmune demyelinating peripheral neuropathies, autoimmune paranodopathies, and autoimmune sensory neuronopathies; autoimmune vasculitis, including, e.g., ANCA-associated vasculitis, PR3-ANCA-associated vasculitis, MPO-ANCA-associated vasculitis, granulomatosis with polyangiitis (GPA), microscopic polyangiitis (MPA), eosinophilic granulomatosis with polyangiitis (EGPA), and renal-limited vasculitis; autoimmune renal diseases, including, e.g., autoimmune glomerulonephritis, membranous nephropathy, and IgA nephropathy; systemic autoimmune and connective tissue diseases, including, e.g., systemic lupus erythematosus (SLE), rheumatoid arthritis (RA), idiopathic inflammatory myopathy (JIM), juvenile dermatomyositis, systemic sclerosis (SSc), mixed connective tissue disease (MCTD), and Sjögren's disease; autoimmune endocrine diseases, including, e.g., type 1 diabetes mellitus (T1DM) and Hashimoto's thyroiditis; autoimmune hepatopancreatobiliary diseases, including, e.g., autoimmune hepatitis, autoimmune pancreatitis, and primary sclerosing cholangitis; autoimmune gastrointestinal diseases, including, e.g., inflammatory bowel disease, Crohn's disease, ulcerative colitis, celiac disease, and dermatitis herpetiformis; autoimmune hematologic diseases, including, e.g., immune thrombocytopenia (ITP) (or idiopathic immune thrombocytopenia), autoimmune hemolytic anemia (AIHA), Evans syndrome, and thrombotic thrombocytopenic purpura; IgG4-related disease; autoimmune blistering and dermatologic diseases, including, e.g., bullous pemphigoid and pemphigus vulgaris; and interstitial lung disease associated with autoimmune or connective tissue diseases, including, e.g., systemic sclerosis-associated interstitial lung disease, rheumatoid arthritis-associated interstitial lung disease, idiopathic inflammatory myopathy-associated interstitial lung disease (including, e.g., dermatomyositis-, polymyositis-, immune-mediated necrotizing myopathy-, and antisynthetase syndrome-associated interstitial lung disease), systemic lupus erythematosus-associated interstitial lung disease, Sjögren's disease-associated interstitial lung disease, mixed connective tissue disease-associated interstitial lung disease, overlap connective tissue disease-associated interstitial lung disease, undifferentiated connective tissue disease-associated interstitial lung disease, ANCA-associated vasculitis-associated interstitial lung disease, and sarcoidosis-associated interstitial lung disease.

In some embodiments, the B cell-mediated disease is an autoimmune disease. In some embodiments, the autoimmune disease is lupus (e.g, systemic lupus erythematosus (SLE), cutaneous lupus, lupus nephritis). In some embodiments, the autoimmune disease is SLE. In some embodiments, the autoimmune disease is idiopathic inflammatory myopathy (JIM). In some embodiments, the autoimmune disease is systemic sclerosis (SSc). In some embodiments, the autoimmune disease is scleroderma. In some embodiments, the autoimmune disease is rheumatoid arthritis (RA). In some embodiments, the autoimmune disease is Sjögren's disease (SjD). In some embodiments, the autoimmune disease is multiple sclerosis. In some embodiments, the autoimmune disease is primary progressive multiple sclerosis (PPMS). In some embodiments, the autoimmune disease is relapsing multiple sclerosis (RMS). In some embodiments, the RMS is relapsing remitting multiple sclerosis (RRMS). In some embodiments, the RMS is active secondary progressive multiple sclerosis (SPMS).

In some embodiments, the autoimmune disease is myasthenia gravis (MG). In some embodiments, the autoimmune disease is autoimmune hemolytic anemia (AIHA). In some embodiments, the autoimmune disease is immune thrombocytopenia (ITP) (or idiopathic immune thrombocytopenia). In some embodiments, the autoimmune disease is Crohn's disease (CD). In some embodiments, the autoimmune disease is ulcerative colitis.

In some embodiments, the autoimmune disease is lupus (e.g., SLE), Sjögren's disease, rheumatoid arthritis, inflammatory myositis, or systemic sclerosis.

In some embodiments, the autoimmune disease comprises interstitial lung disease associated with a rheumatologic or connective-tissue disease, including, e.g., systemic sclerosis-associated interstitial lung disease, rheumatoid arthritis-associated interstitial lung disease, idiopathic inflammatory myopathy-associated interstitial lung disease (including dermatomyositis-, polymyositis-, immune-mediated necrotizing myopathy-, and antisynthetase syndrome-associated interstitial lung disease), systemic lupus erythematosus-associated interstitial lung disease, Sjögren's syndrome-associated interstitial lung disease, mixed connective tissue disease-associated interstitial lung disease, overlap connective tissue disease-associated interstitial lung disease, undifferentiated connective tissue disease-associated interstitial lung disease, ANCA-associated vasculitis-associated interstitial lung disease, and sarcoidosis-associated interstitial lung disease. In some embodiments, the autoimmune disease is interstitial lung disease.

In some embodiments, the autoimmune disease is a systemic sclerosis-associated interstitial lung disease. In some embodiments, the autoimmune disease is a rheumatoid arthritis-associated interstitial lung disease. In some embodiments, the autoimmune disease is a idiopathic inflammatory myopathy-associated interstitial lung disease (including dermatomyositis-, polymyositis-, immune-mediated necrotizing myopathy-, and antisynthetase syndrome-associated interstitial lung disease). In some embodiments, the autoimmune disease is a systemic lupus erythematosus-associated interstitial lung disease. In some embodiments, the autoimmune disease is a Sjögren's syndrome-associated interstitial lung disease. In some embodiments, the autoimmune disease is a mixed connective tissue disease-associated interstitial lung disease. In some embodiments, the autoimmune disease is an overlap connective tissue disease-associated interstitial lung disease. In some embodiments, the autoimmune disease is an undifferentiated connective tissue disease-associated interstitial lung disease. In some embodiments, the autoimmune disease is an ANCA-associated vasculitis-associated interstitial lung disease. In some embodiments, the autoimmune disease is a sarcoidosis-associated interstitial lung disease.

In some embodiments, the autoimmune disease is cutaneous lupus erythematosus (CLE). In some embodiments, the autoimmune disease is lupus nephritis (LN). In some embodiments, the autoimmune disease is idiopathic inflammatory myopathy (JIM) or myositis. In some embodiments, the autoimmune disease is antisynthetase syndrome. In some embodiments, the autoimmune disease is pemphigus. In some embodiments, the autoimmune disease is pemphigus vulgaris. In some embodiments, the autoimmune disease is juvenile dermatomyositis. In some embodiments, the autoimmune disease is ANCA associated vasculitis. In some embodiments, the autoimmune disease is autoimmune vasculitis. In some embodiments, the autoimmune disease is granulomatosis with polyangitis (GPA). In some embodiments, the autoimmune disease is Type I diabetes (T1DM). In some embodiments, the autoimmune disease is autoimmune hemolytic anemia (AIHA). In some embodiments, the autoimmune disease is immune thrombocytopenic purpura (ITP). In some embodiments, the autoimmune disease is Hashimoto's thyroiditis. In some embodiments, the autoimmune disease is Stiff Person Syndrome (SPS). In some embodiments, the autoimmune disease is autoimmune hepatitis. In some embodiments, the autoimmune disease is autoimmune pancreatitis. In some embodiments, the autoimmune disease is celiac disease. In some embodiments, the autoimmune disease is dermatitis herpetiformis. In some embodiments, the autoimmune disease is primary sclerosing cholangitis. In some embodiments, the autoimmune disease is autoimmune glomerulonephritis. In some embodiments, the autoimmune disease is IgA nephropathy. In some embodiments, the autoimmune disease is neuromyelitis optica.

In some embodiments, the autoimmune disease is autoimmune inner ear disease. In some embodiments, the autoimmune disease is Cogan's syndrome. In some embodiments, the autoimmune disease is Meniere's disease. In some embodiments, the autoimmune disease is autoimmune dysautonomia. In some embodiments, the autoimmune disease is chronic Lyme disease. In some embodiments, the autoimmune disease is fibromyalgia. In some embodiments, the autoimmune disease is interstitial cystitis. In some embodiments, the autoimmune disease is narcolepsy. In some embodiments, the autoimmune disease is alopecia areata. In some embodiments, the autoimmune disease is autoimmune urticaria. In some embodiments, the autoimmune disease is a benign mucosal pemphigoid. In some embodiments, the autoimmune disease is bullous pemphigoid. In some embodiments, the autoimmune disease is a cicatricial pemphigoid. In some embodiments, the autoimmune disease is hidradenitis suppurativa. In some embodiments, the autoimmune disease is lichen planus. In some embodiments, the autoimmune disease is lichen sclerosus. In some embodiments, the autoimmune disease is pityriasis lichenoides et varioliformis acuta (PLEVA). In some embodiments, the autoimmune disease is pyoderma gangrenosum. In some embodiments, the autoimmune disease is vitiligo. In some embodiments, the autoimmune disease is Addison's disease. In some embodiments, the autoimmune disease is Grave's disease. In some embodiments, the autoimmune disease is polyglandular syndromes types I, II, III. In some embodiments, the autoimmune disease is thyroid eye disease. In some embodiments, the autoimmune disease is eosinophilic esophagitis. In some embodiments, the autoimmune disease is primary biliary cholangitis. In some embodiments, the autoimmune disease is primary sclerosing cholangitis. In some embodiments, the autoimmune disease is autoimmune oophoritis. In some embodiments, the autoimmune disease is autoimmune orchitis. In some embodiments, the autoimmune disease is herpes gestationis. In some embodiments, the autoimmune disease is progesterone dermatitis. In some embodiments, the autoimmune disease is endometriosis. In some embodiments, the autoimmune disease is agammaglobulinemia. In some embodiments, the autoimmune disease is amyloidosis. In some embodiments, the autoimmune disease is autoimmune angioedema. In some embodiments, the autoimmune disease is autoimmune hemolytic anemia. In some embodiments, the autoimmune disease is autoimmune neutropenia. In some embodiments, the autoimmune disease is autoimmune thrombocytopenic purpura. In some embodiments, the autoimmune disease is Castleman disease. In some embodiments, the autoimmune disease is cold agglutinin disease. In some embodiments, the autoimmune disease is essential mixed cryoglobulinemia. In some embodiments, the autoimmune disease is paroxysmal nocturnal hemoglobinuria. In some embodiments, the autoimmune disease is pernicious anemia. In some embodiments, the autoimmune disease is POEMS syndrome. In some embodiments, the autoimmune disease is pure red cell aplasia. In some embodiments, the autoimmune disease is antiphospholipid syndrome. In some embodiments, the autoimmune disease is congenital heart block, In some embodiments, the autoimmune disease is CREST syndrome. In some embodiments, the autoimmune disease is limited scleroderma. In some embodiments, the autoimmune disease is dermatomyositis. In some embodiments, the autoimmune disease is discoid lupus. In some embodiments, the autoimmune disease is eosinophilic fascitis. In some embodiments, the autoimmune disease is Evans syndrome. In some embodiments, the autoimmune disease is inclusion body myositis. In some embodiments, the autoimmune disease is juvenile myositis. In some embodiments, the autoimmune disease is mixed connective tissue disease. In some embodiments, the autoimmune disease is neonatal lupus. In some embodiments, the autoimmune disease is progressive hemifacial atrophy. In some embodiments, the autoimmune disease is Raynaud's phenomenon. In some embodiments, the autoimmune disease is anti-myelin oligodendrocyte glycoprotein (MOG) autoimmune encephalitis. In some embodiments, the autoimmune disease is Bald disease. In some embodiments, the autoimmune disease is chronic inflammatory demyelinating polyneuropathy. In some embodiments, the autoimmune disease is complex regional pain syndrome. In some embodiments, the autoimmune disease is Guillan-Barre syndrome. In some embodiments, the autoimmune disease is Lambert-Eaton syndrome. In some embodiments, the autoimmune disease is multifocal motor neuropathy. In some embodiments, the autoimmune disease is optic neuritis. In some embodiments, the autoimmune disease is paraneoplastic cerebellar degeneration. In some embodiments, the autoimmune disease is brachial neuritis. In some embodiments, the autoimmune disease is Parsonage-Turner syndrome. In some embodiments, the autoimmune disease is perivenous encephalomyelitis. In some embodiments, the autoimmune disease is transverse myelitis. In some embodiments, the autoimmune disease is autoimmune retinopathy. In some embodiments, the autoimmune disease is autoimmune uveitis. In some embodiments, the autoimmune disease is pars planitis. In some embodiments, the autoimmune disease is scleritis.

In some embodiments, the autoimmune disease is sympathetic ophthalmia. In some embodiments, the autoimmune disease is Tolosa-Hunt syndrome. In some embodiments, the autoimmune disease is erythema nodosum. In some embodiments, the autoimmune disease is fibrosing alveolitis. In some embodiments, the autoimmune disease is pulmonary alveolar proteinosis. In some embodiments, the autoimmune disease is sarcoidosis. In some embodiments, the autoimmune disease is Goodpasture's syndrome. In some embodiments, the autoimmune disease is Henoch-Schonlein purpura. In some embodiments, the autoimmune disease is linear IgA disease. In some embodiments, the autoimmune disease is ankylosing spondylitis. In some embodiments, the autoimmune disease is juvenile idiopathic arthritis. In some embodiments, the autoimmune disease is palindromic rheumatism. In some embodiments, the autoimmune disease is psoriasis. In some embodiments, the autoimmune disease is psoriatic arthritis. In some embodiments, the autoimmune disease is reactive arthritis. In some embodiments, the autoimmune disease is Adult Still's disease. In some embodiments, the autoimmune disease is chronic recurrent multifocal osteomyelitis.

In some embodiments, the autoimmune disease is giant cell myocarditis. In some embodiments, the autoimmune disease is Kawasaki disease. In some embodiments, the autoimmune disease is myocarditis. In some embodiments, the autoimmune disease is pediatric autoimmune neuropsychiatric disorders associated with streptococcus infections (PANDAS). In some embodiments, the autoimmune disease is postmyocardial infarction syndrome. In some embodiments, the autoimmune disease is Dressler's syndrome. In some embodiments, the autoimmune disease is rheumatic fever. In some embodiments, the autoimmune disease is Behcet's disease. In some embodiments, the autoimmune disease is Churg-Strauss syndrome. In some embodiments, the autoimmune disease is giant cell arteritis. In some embodiments, the autoimmune disease is granulomatosis with polyangiitis. In some embodiments, the autoimmune disease is IgG4-related sclerosing disease. In some embodiments, the autoimmune disease is microscopic polyangiitis. In some embodiments, the autoimmune disease is polyarteritis nodosa. In some embodiments, the autoimmune disease is relapsing polychondritis. In some embodiments, the autoimmune disease is polymyalgia rheumatica. In some embodiments, the autoimmune disease is retroperitoneal fibrosis. In some embodiments, the autoimmune disease is Susac's syndrome. In some embodiments, the autoimmune disease is Takayasu's arteritis. In some embodiments, the autoimmune disease is Vogt-Koyanagi-Harada disease.

### Cancer

In some embodiments, the B cell-mediated disease is a cancer. Targeted (e.g., CD19-targeted) CAR T-cell therapies can target malignancies characterized by expression of the target surface antigen (e.g., CD19) and are applicable across a broad range of B-cell neoplasms. CD19-expressing malignancies include B-cell acute lymphoblastic leukemia and non-Hodgkin lymphomas such as diffuse large B-cell lymphoma, high-grade B-cell lymphoma, primary mediastinal large B-cell lymphoma, follicular lymphoma, mantle cell lymphoma, marginal zone lymphoma, and lymphomas. In some embodiments, the cancer is a hematologic malignancy. In some embodiments, the cancer is a B-cell acute lymphoblastic leukemia. In some embodiments, the cancer is a non-Hodgkin lymphoma (NHL). In some embodiments, the cancer is a diffuse large B-cell lymphoma. In some embodiments, the cancer is a high-grade B-cell lymphoma. In some embodiments, the cancer is a primary mediastinal large B-cell lymphoma. In some embodiments, the cancer is a follicular lymphoma. In some embodiments, the cancer is a mantle cell lymphoma. In some embodiments, the cancer is a marginal zone lymphoma. In some embodiments, the cancer is a transformed lymphoma. In some embodiments, the cancer is a chronic lymphocytic leukemia. In some embodiments, the cancer is a small lymphocytic lymphoma. In some embodiments, the cancer is a multiple myeloma (MM).

### Compositions and Kits

In some aspects, provided herein is a circRNA therapeutic, comprising circRNA of the present disclosure. In some embodiments, the circRNA is as provided by the present disclosure.

In some embodiments, the circRNA therapeutic comprises the circRNA of the present disclosure encapsulated in an LNP. In some embodiments, the LNP is conjugated to a targeting moiety, wherein the targeting moiety binds to T cells.

In some aspects, provided herein is a circRNA therapeutic for targeted delivery, comprising: (i) a circRNA comprising (a) a 5′ untranslated region (UTR), (b) an Internal Ribosome Entry Site (IRES) having at least 85% identity to SEQ ID NO: 1, (c) a coding sequence encoding: a CD8a signal peptide, and a Chimeric Antigen Receptor (CAR) comprising: an anti-CD19 scFv, a CD8a hinge, a CD8a transmembrane domain, a 4-1BB costimulatory domain, a CD3 zeta signaling domain, and (d) a 3′ UTR, and (ii) an anti-CD5 T cell targeting moiety comprising the complementarity determining region (CDR) sequences of GFTFSMYS (SEQ ID NO:64), ISTGARDT (SEQ ID NO: 65) and GDLRYGPDGYDY (SEQ ID NO: 66), wherein the circRNA is encapsulated in a lipid nanoparticle, and wherein the anti-CD5 T cell targeting moiety is conjugated to the lipid nanoparticle. In some embodiments, the CD8a hinge comprises a sequence having at least 75%, 80%, 85%, 90%, 95% or 99% identity to SEQ ID NO: 285; the CD8a transmembrane domain comprises a sequence having at least 75%, 80%, 85%, 90%, 95% or 99% identity to SEQ ID NO: 287; the 4-1BB costimulatory domain comprises a sequence having at least 75%, 80%, 85%, 90%, 95% or 99% identity to SEQ ID NO: 293, and/or the CD3z signaling domain comprises a sequence having at least 75%, 80%, 85%, 90%, 95% or 99% identity to SEQ ID NO: 294. In some embodiments, the CD8a hinge comprises a sequence of SEQ ID NO: 285; the CD8a transmembrane domain comprises a sequence of SEQ ID NO: 287; the 4-1BB costimulatory domain comprises a sequence of SEQ ID NO: 293, and/or the CD3z signaling domain comprises a sequence of SEQ ID NO: 294.

In some aspects, provided herein is a circRNA therapeutic, comprising: (i) a circRNA comprising (a) a 5′ UTR, (b) an IRES having at least 85% identity to a sequence comprising SEQ ID NO: 1, (c) a coding sequence encoding a chimeric antigen receptor (CAR), (d) a 3′ UTR, and (ii) an anti-CD5 T cell targeting moiety comprising the complementarity determining region (CDR) sequences CDR1, CDR2, and CDR3, wherein the CDR1, the CDR2, and the CDR3 amino acid sequences are GFTFSMYS (SEQ ID NO:64), ISTGARDT (SEQ ID NO: 65), and GDLRYGPDGYDY (SEQ ID NO: 66), respectively, wherein the circRNA is encapsulated in a lipid nanoparticle, and wherein the anti-CD5 T cell targeting moiety is conjugated to the lipid nanoparticle.

In some embodiments, the IRES comprises a nucleic acid sequence having at least 90% identity to a sequence comprising SEQ ID NO: 1.

In some embodiments, the IRES comprises a nucleic acid sequence having 100% identity to SEQ ID NO: 1.

In some embodiments, the 3′ UTR comprises SEQ ID NO: 46 or SEQ ID NO: 47 with one or more microRNA binding sites.

In some embodiments, the 5′ UTR comprises SEQ ID NO: 265.

In some embodiments, the 3′ UTR has one miR-122 binding site.

In some embodiments, the anti-CD19 scFv comprises: a VL sequence of

(SEQ ID NO: 54)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIT,

linked by a linker having the amino acid sequence of GGGGSGGGGSGGGGS (SEQ ID NO: 56), and a VH sequence of EVKLQESGPGLVAPSQSLSVTCTVSGVSLPDYGVSWIRQPPR KGLEWLGVIWGSETTYYNSALKSRLTIIKDNSKSQVFLKMNSLQTDDTAIYYCAKHYYY GGSYAMDYWGQGTSVTVSS (SEQ ID NO: 55).

In some embodiments of the circRNA therapeutic for targeted delivery, the LNP comprises one or more ionizable lipids, one or more phospholipids, one or more sterol lipids, and two PEG-lipids at a ratio of 39:20:39:1.9:0.1 mol %.

In some embodiments of the circRNA therapeutic for targeted delivery, the LNP comprises an ionizable lipid, a phospholipid, a sterol lipid, and two PEG-lipids at a ratio of 39:20:39:1.9:0.1 mol %.

In some embodiments, the ionizable lipid is:

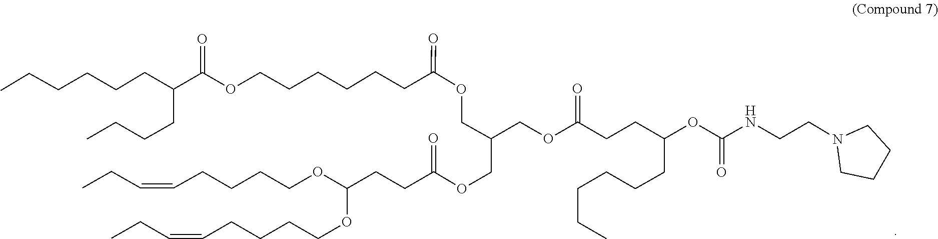 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN1CCCC1)OCCCC/C=C\CC]

In some embodiments, the T-cell targeting moiety is conjugated via a thiol-maleimide conjugation.

In some aspects, provided herein is a circRNA therapeutic for targeted delivery, comprising:

(i) a circRNA comprising (a) a 5′ untranslated region (UTR), (b) an Internal Ribosome Entry Site (IRES) comprises a nucleic acid sequence comprising a sequence having 100% identity to SEQ ID NO: 1, (c) a coding sequence encoding: a Chimeric Antigen Receptor (CAR) comprising: FMC63 anti-CD19 scFv having a VL sequence of

(SEQ ID NO: 54)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIT,

linked by a linker having the amino acid sequence of GGGGSGGGGSGGGGS (SEQ ID NO: 56) and a VH sequence of EVKLQESGPGLVAPSQSLSVTCTVSGVSLPDYGVSWIRQPPRKG LEWLGVIWGSETTYYNSALKSRLTIIKDNSKSQVFLKMNSLQTDDTAIYYCAKHYYYGG SYAMDYWGQGTSVTVSS (SEQ ID NO: 55), a CD8a hinge, a CD8a transmembrane domain, a 4-1BB costimulatory domain, a CD3 zeta signaling domain, and a CD8 signal peptide; and (d) a 3′ untranslated region (UTR) of SEQ ID NO: 46 comprising a miR-122 site, and (ii) an anti-CD5 T cell targeting moiety comprising the sequence of SEQ ID NO: 89, wherein the circRNA is encapsulated in a lipid nanoparticle, wherein the anti-CD5 T-cell targeting moiety is conjugated to the lipid nanoparticle via a thiol-maleimide conjugation.

In some embodiments, the CD8a hinge comprises a sequence having at least 75%, 80%, 85%, 90%, 95% or 99% identity to SEQ ID NO: 285; the CD8a transmembrane domain comprises a sequence having at least 75%, 80%, 85%, 90%, 95% or 99% identity to SEQ ID NO: 287; the 4-1BB costimulatory domain comprises a sequence having at least 75%, 80%, 85%, 90%, 95% or 99% identity to SEQ ID NO: 293, and/or the CD3z signaling domain comprises a sequence having at least 75%, 80%, 85%, 90%, 95% or 99% identity to SEQ ID NO: 294. In some embodiments, the CD8a hinge comprises a sequence of SEQ ID NO: 285; the CD8a transmembrane domain comprises a sequence of SEQ ID NO: 287; the 4-1BB costimulatory domain comprises a sequence of SEQ ID NO: 293, and/or the CD3z signaling domain comprises a sequence of SEQ ID NO: 294.

In some embodiments, the LNP comprises an ionizable lipid, a phospholipid, a sterol lipid, and one or more PEG-lipids.

In some embodiments, the ionizable lipid is:

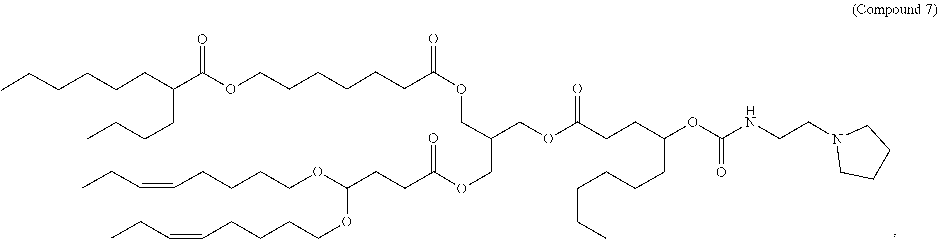 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN1CCCC1)OCCCC/C=C\CC]

and wherein the ionizable lipid, the phospholipid, the sterol lipid, DMG-PEG 2000 lipid, and DSPE-PEG 2000-maleimide is at a ratio of 39: 20: 39: 1.9: 0.1 mol %.

In some embodiments, provided herein is a composition comprising the circRNA therapeutic for targeted delivery, the circRNA for targeted delivery, or the circRNA of the present disclosure.

In some embodiments, provided herein is a composition comprising a circRNA (encoding an anti-CD19 CAR) for targeted delivery. In some embodiments, the composition comprises the vector, host cell or any combination thereof.

In some embodiments, the composition is a pharmaceutical composition comprising a therapeutically effective amount of the circRNA encoding a CAR, e.g., an anti-CD19 CAR. A pharmaceutically acceptable vehicle may include any and all carriers, solvents, diluents, excipients, adjuvants, dispersion media, coatings, antibacterial or antifungal agents, adsorption delaying agents and the like, suitable for human administration. As used herein, the composition is administered in a therapeutically effective amount which is a dose sufficient to achieve a therapeutic effect.

Provided herein is a kit comprising the circRNA of the present disclosure, wherein the circRNA comprises a ribosome recruiting element, and a coding sequence encoding a chimeric antigen receptor (CAR) comprising an anti-CD19 antibody or antigen-binding fragment thereof. Provided herein is a kit comprising the precursor nucleic acid to make the circRNA of the present disclosure. Provided herein is a kit comprising the precursor RNA to make the circRNA of the present disclosure. In some embodiments, the kit comprises the circRNA of the present disclosure as well as a targeting moiety, wherein the targeting moiety binds to an immune cell (e.g., T cell), and a delivery vehicle, such as a lipid nanoparticle or virus like particle.

### NUMBERED EMBODIMENTS

1. A targeted lipid nanoparticle, comprising:
(i) a lipid nanoparticle (LNP), and
(ii) a targeting moiety comprising a VHH that is conjugated to the LNP;
   wherein the LNP comprises one or more ionizable lipids at 30-49 mol %, one or more phospholipids at 5-40 mol %, one or more sterol lipids at 15-50 mol %, a PEG-lipid at 0-10 mol %, and a conjugating PEG-lipid at 0.005-1 mol %.
   2. The targeted LNP of numbered embodiment 1, wherein the LNP comprises the one or more ionizable lipids at 30 to 45 mol %.
   3. The targeted LNP of numbered embodiment 1 or 2, wherein the LNP comprises the one or more phospholipids at 15 to 35 mol %.
   4. The targeted LNP of any one of numbered embodiments 1 to 3, wherein the LNP comprises the one or more sterol lipids at 30 to 50 mol %.
   5. The targeted LNP of any one of numbered embodiments 1 to 4, wherein the LNP comprises the PEG lipid at 0 to 5 mol %.
   6. The targeted LNP of any one of numbered embodiments 1 to 5, wherein the LNP comprises the conjugating PEG-lipid at 0.01 to 1 mol %.
   7. The targeted LNP of any one of numbered embodiments 1 to 6, wherein the LNP is formed by combining the one or more ionizable lipids, the one or more phospholipids, the one or more sterol lipids, the PEG-lipid and the conjugating PEG-lipid at a ratio of 39: 20: 39: 1.9 0.1 mol %.
   8. The targeted LNP of any one of the preceding numbered embodiments, wherein the one or more ionizable lipids comprises Compound 7.
   9. The targeted LNP of numbered embodiment 8, wherein LNP comprises at least 30 mol % Compound 7.
   10. The targeted LNP of any one of numbered embodiments 1 to 9, wherein the one or more ionizable lipids is Compound 7.
   11. The targeted LNP of any one of numbered embodiments 1 to 10, wherein the one or more phospholipids comprises 1,2-distearoyl-snglycero-3-phosphocholine (DSPC).
   12. The targeted LNP of numbered embodiment 11, wherein the LNP comprises at least 15 mol % DSPC.
   13. The targeted LNP of any one of numbered embodiments 1 to 12, wherein the one or more phospholipids is DSPC.
   14. The targeted LNP of any one of numbered embodiments 1 to 13, wherein the one or more sterol lipids comprises cholesterol.
   15. The targeted LNP of numbered embodiment 14, wherein the LNP comprises at least 15 mol % cholesterol.
   16. The targeted LNP of any one of numbered embodiments 1 to 15, the LNP comprises at least 30 mol % cholesterol.
   17. The targeted LNP of any one of numbered embodiments 1 to 16, wherein the one or more sterol lipids is cholesterol.
   18. The targeted LNP of any one of numbered embodiments 1 to 17, wherein the conjugating PEG-lipid comprises a long chain fatty acid.
   19. The targeted LNP of numbered embodiment 18, wherein the long chain fatty acid is between C14 and C22.
   20. The targeted LNP of numbered embodiment 19, wherein the long chain fatty acid is C18.
   21. The targeted LNP of any one of numbered embodiments 18 to 20, wherein LNP comprises at least 0.005% of the long chain fatty acid.
   22. The targeted LNP of any one of numbered embodiments 18 to 20, wherein LNP comprises at least 0.01% of the long chain fatty acid.
   23. The targeted LNP of any one of numbered embodiments 1 to 22, wherein the conjugating PEG-lipid is a DSPE-PEG-2000 lipid.
   24. The targeted LNP of any one of numbered embodiments 1 to 17, wherein the PEG-lipid is a cholesterol-PEG-2000 lipid.
   25. The targeted LNP of any one of numbered embodiments 1 to 23, wherein the conjugating PEG-lipid comprises DSPE-PEG 2000-maleimide.
   26. The targeted LNP of numbered embodiment 25, wherein the LNP comprises at least 0.005 mol % DSPE-PEG 2000-maleimide.
   27. The targeted LNP of numbered embodiment 25, wherein the LNP comprises at least 0.01 mol % DSPE-PEG 2000-maleimide.
   28. The targeted LNP of any one of numbered embodiments 23 to 27, wherein the conjugating PEG-lipid is DSPE-PEG 2000-maleimide.
   29. The targeted LNP of any one of numbered embodiments 1 to 28, wherein VHH is conjugated to the LNP via a conjugation handle that is attached to the conjugating PEG-lipid.
   30. The targeted LNP of numbered embodiment 29, wherein C-terminus of the VHH is covalently attached to the N terminus of the conjugation handle.
   31. The targeted LNP of numbered embodiment 29 or 30, wherein the conjugation handle comprises any one of SEQ ID NOs: 117-121 and SEQ ID NOs: 343-345.
   32. The targeted LNP of numbered embodiment 31, wherein the conjugation handle comprises any one of SEQ ID NO: 120, 343 and 345.
   33. The targeted LNP of any one of numbered embodiments 29-30 and 32, wherein the VHH is conjugated to the LNP via a thiol-maleimide conjugation.
   34. The targeted LNP of numbered embodiment 33, wherein the conjugation handle comprises SEQ ID NO: 120.
   35. The targeted LNP of numbered embodiment 34, wherein the conjugation handle consists of SEQ ID NO: 120.
   36. The targeted LNP of any one of numbered embodiments 1 to 35, wherein the VHH has a length of 110 to 130 (e.g., 115 to 125) 120-140 amino acids (e.g., about 121 amino acids).
   37. The targeted LNP of any one of numbered embodiments 1 to 36, wherein the VHH with attached conjugation handle has a length of 120 to 140 (e.g., 125 to −135) amino acids.
   38. The targeted LNP of any one of numbered embodiments 1 to 37, wherein the VHH with attached conjugation handle has a length of about 129 amino acids.
   39. The targeted LNP of any one of numbered embodiments 1 to 38, wherein the VHH is an anti-CD5 VHH.
   40. The targeted LNP of numbered embodiment 39, wherein the VHH comprises CDR1, CDR2, and CDR3 sequences from SEQ ID NO: 89.
   41. The targeted LNP of numbered embodiment 40, wherein the CDR1 sequence comprises SEQ ID NO: 64, the CDR2 sequence comprises SEQ ID NO: 65, and the CDR3 sequence comprises SEQ ID NO: 66.
   42. The targeted LNP of numbered embodiment 40, wherein the CDR1 sequence comprises SEQ ID NO: 237, the CDR2 sequence comprises SEQ ID NO: 238, and the CDR3 sequence comprises SEQ ID NO: 239.
   43. The targeted LNP of any one of numbered embodiments 39 to 42, wherein the VHH is at least 80% identical to SEQ ID NO: 89.
   44. The targeted LNP of any one of numbered embodiments 39 to 42, wherein the VHH is at least 85% identical to SEQ ID NO: 89.
   45. The targeted LNP of any one of numbered embodiments 39 to 42, wherein the VHH is at least 90% identical to SEQ ID NO: 89.
   46. The targeted LNP of any one of numbered embodiments 39 to 42, wherein the VHH is at least 95% identical to SEQ ID NO: 89.
   47. The targeted LNP of numbered embodiment 46, wherein the VHH antibody comprises the sequence of SEQ ID NO: 89.
   48. The targeted LNP of numbered embodiment 47, wherein the sequence of the anti-CD5 VHH antibody with the conjugation handle comprises a sequence selected from SEQ ID NOs: 122-124.
   49. The targeted LNP of numbered embodiment 47, wherein the sequence of the anti-CD5 VHH antibody with the conjugation handle consists of a sequence selected from SEQ ID NOs: 122-124.
   50. The targeted LNP of any one of numbered embodiments 1 to 49, wherein the targeting moiety comprises an anti-CD2 antibody or antigen-binding fragment thereof, an anti-CD3 antibody or antigen-binding fragment thereof, an anti-CD7 antibody or antigen-binding fragment thereof, or an anti-CD8 antibody or antigen-binding fragment thereof.
   51. A targeted lipid nanoparticle comprising:
an LNP conjugated to a targeting moiety,
wherein the targeting moiety is an anti-CD5 VHH, the anti-CD5 VHH comprising CDR1, CDR2, and CDR3 sequences from SEQ ID NO: 89, wherein the LNP comprises one or more ionizable lipids at 30 to 49 mol %, one or more phospholipids at 5 to 40 mol %, one or more sterol lipids at 15 to 50 mol %, DMG-PEG 2000 at 0 to 10 mol %, and DSPE-PEG 2000-maleimide at 0.005 to 1 mol % (e.g., at 0.01 to 1 mol %),
wherein the VHH is conjugated to the LNP via a thiol-maleimide conjugation.
   52. The targeted LNP of numbered embodiment 51, wherein the thiol-maleimide conjugation is between the DSPE-PEG 2000-maleimide and a conjugation handle that is attached to the VHH.
   53. The targeted LNP of numbered embodiment 51 or 52, wherein the N-terminus of the conjugation handle is covalently attached to the C-terminus of the VHH.
   54. The targeted LNP of any one of numbered embodiments 51 to 53, wherein the anti-CD5 VHH is at least 95% identical to SEQ ID NO:89.
   55. The targeted LNP of any one of numbered embodiments 51 to 54, wherein the conjugation handle comprises SEQ ID NO:120.
   56. The targeted LNP of any one of numbered embodiments 51 to 55, wherein the LNP comprises the one or more ionizable lipids at 30 to 45 mol %.
   57. The targeted LNP of any one of numbered embodiments 51 to 56, wherein the LNP comprises the one or more phospholipids at 15 to 35 mol %.
   58. The targeted LNP of any one of numbered embodiments 51 to 57, wherein the LNP comprises the one or more sterol lipids at 30 to 50 mol %.
   59. The targeted LNP of any one of numbered embodiments 51 to 58, wherein the LNP comprises the DMG-PEG 2000 at 0 to 5 mol %.
   60. The targeted LNP of any one of numbered embodiments 51 to 59, wherein the LNP is formed by combining the one or more ionizable lipids, the one or more phospholipids, the one or more sterol lipids, the DMG-PEG 2000, and the DSPE-PEG 2000-maleimide at a molar ratio of 39:20: 39: 1.9:0.1.
   61. The targeted LNP of any one of numbered embodiments 51 to 60, wherein the one or more ionizable lipids comprises Compound 7.
   62. The targeted LNP of numbered embodiment 61, wherein LNP comprises at least 30 mol % Compound 7.
   63. The targeted LNP of any one of numbered embodiments 51 to 62, wherein the one or more ionizable lipids is Compound 7.
   64. The targeted LNP of any one of numbered embodiments 51 to 63, wherein the one or more phospholipids comprises DSPC.
   65. The targeted LNP of numbered embodiment 64, wherein the LNP comprises at least 15 mol % DSPC.
   66. The targeted LNP of any one of numbered embodiments 51 to 65, wherein the one or more phospholipids is DSPC.
   67. The targeted LNP of any one of numbered embodiments 51 to 66, wherein the one or more sterol lipids comprises cholesterol.
   68. The targeted LNP of numbered embodiment 67, wherein the LNP comprises at least 15 mol % cholesterol.
   69. The targeted LNP of any one of numbered embodiments 51 to 68, the LNP comprises at least 30 mol % cholesterol.
   70. The targeted LNP of any one of numbered embodiments 51 to 69, wherein the one or more sterol lipids is cholesterol.
   71. The targeted LNP of any one of numbered embodiments 1 to 70, wherein the LNP is targeted to T cells.
   72. The targeted LNP of any one of numbered embodiments 1 to 71, further comprising a payload.
   73. The targeted LNP of numbered embodiment 72, wherein the payload comprises one or more polypeptides.
   74. The targeted LNP of any one of numbered embodiments 1 to 73, the payload comprising one or more nucleic acids.
   75. The targeted LNP of numbered embodiment 74, wherein the one or more nucleic acids is DNA and/or RNA.
   76. The targeted LNP of numbered embodiment 75, wherein the one or more nucleic acids is DNA.
   77. The targeted LNP of numbered embodiment 79, wherein the one or more nucleic acids is RNA.
   78. The targeted LNP of numbered embodiment 75 or 77, wherein the RNA comprises a linear RNA.
   79. The targeted LNP of numbered embodiment 78, wherein the linear RNA is a messenger RNA or noncoding RNA.
   80. The targeted LNP of numbered embodiment 75 or 77, wherein the RNA comprises a circular RNA (e.g., a circRNA disclosed herein).
   81. The targeted LNP of numbered embodiment 80, wherein the one or more nucleic acids comprises oligonucleotides.
   82. The targeted LNP of numbered embodiment 74, wherein the payload comprises or encodes a chimeric antigen receptor (CAR).

### EXAMPLES

Various aspects of the invention are described in further detail in the following Examples. The following examples describe some of the exemplary modes of making and practicing the present invention. However, it should be understood that these Examples are for illustrative purposes only and are not meant to limit the scope of the invention.

### Example 1. Exemplary circRNA Designs Encapsulated in Targeted Lipid Nanoparticles for Targeted Delivery to T Cells

This example illustrates the designs of exemplary circRNAs and precursor RNA comprising various optimized elements for, e.g., circularization efficiency and expression in T cells. This example also illustrates the precise engineering of circRNA encapsulated in lipid nanoparticles for targeted delivery to T cells.

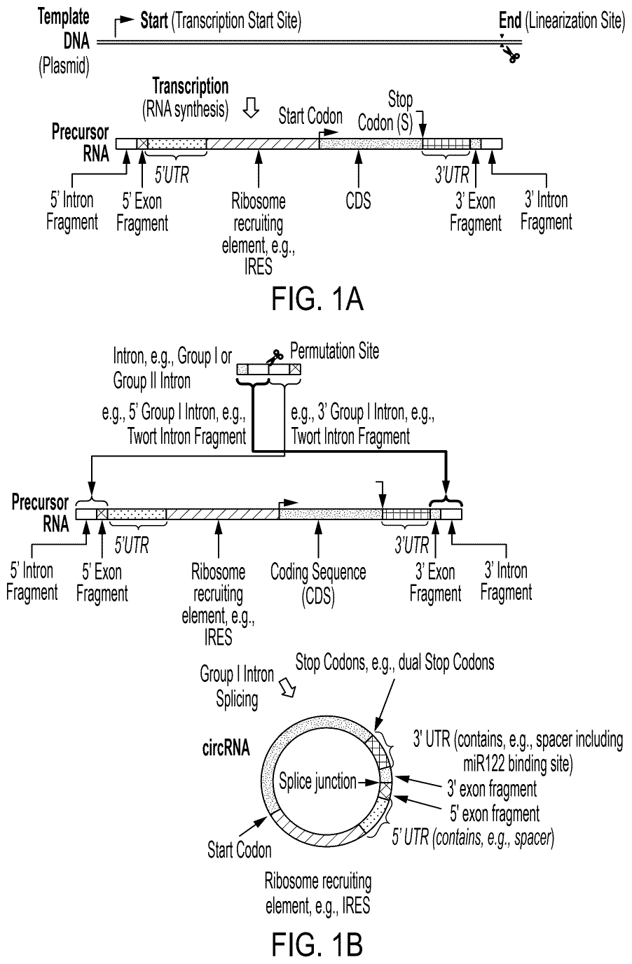

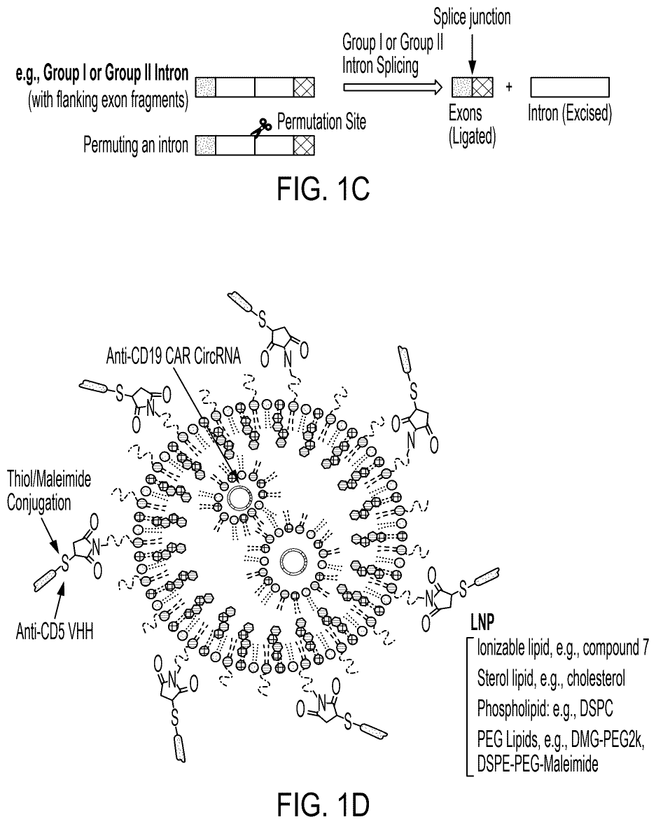

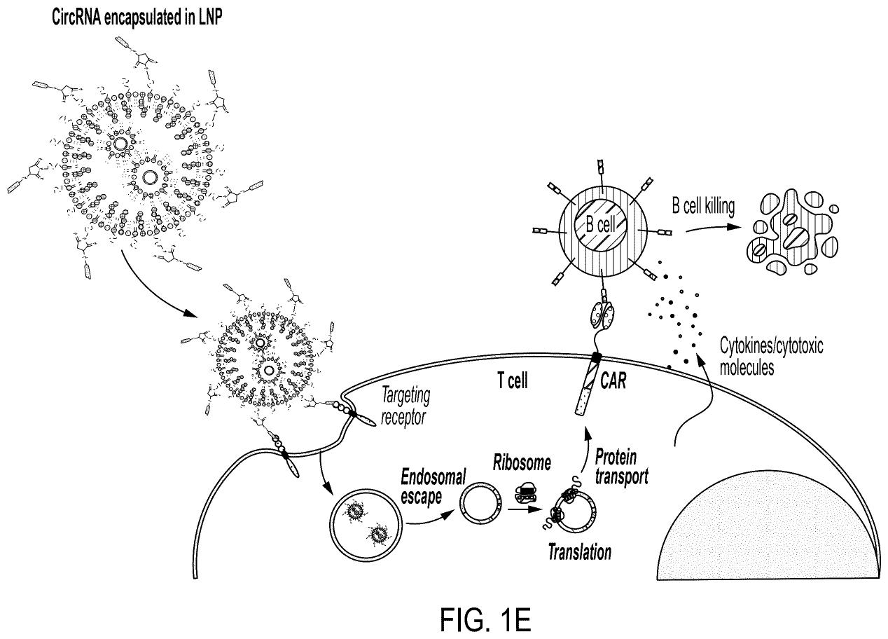

FIG. 1A shows a schematic showing a template DNA (plasmid), which is transcribed to synthesize an exemplary linear precursor RNA (from which the circular RNA of the present disclosure is derived). In some embodiments, as shown in FIG. 1B, in a precursor RNA, the sequence to be circularized is flanked by fragments of a permuted self-splicing intron (e.g., Group I intron, such as a Twort intron). Autocatalytic self-splicing of two halves of a permuted Group I intron-exon (PIE) construct leads to formation of the single-stranded circular RNA. As shown in more detail in FIG. 1C, after autocatalytic self-splicing of two halves of a PIE construct, the 5′ exon fragment and 3′ exon fragment are ligated, and the intron is excised from the circular RNA.

In some embodiments, the circRNA drug substance of the present disclosure encodes an anti-CD19 chimeric antigen receptor (CAR) for treatment of B-cell-driven diseases (e.g., autoimmune disease and cancer). As shown in FIG. 1B, in some embodiments, the circRNA sequence is comprised of a coding sequence (CDS) that encodes a CAR, a ribosome recruiting element, e.g., an internal ribosome entry site (IRES), 5′ and 3′ untranslated region(s) (UTR) optionally containing one or more spacers, and 5′ and 3′ exon fragment(s) joined at a splice junction. In some embodiments, the circRNA is also engineered to minimize off-target expression, e.g., through the use of miRNA binding sites (engineered within spacers in the 5′ and/or 3′ UTR of the circRNA). For example, miR-122 is highly expressed in the liver, and is the most abundant miRNA in the adult liver. The use of miRNA binding sites, such as miR-122 binding sites, in the circRNA decreases CAR expression in the liver, which is an undesirable off-target effect. The miRNA binding site in the circRNA binds to the miRNA in the liver, which leads to translation inhibition, thus reducing expression of the CAR in the liver.

In some embodiments, the nucleobase composition of the circRNA drug substance is adenine, cytosine, guanine, and uracil. In some embodiments, the circRNA is between 1500-3000 nucleotides long. In some embodiments, the circRNA is 2000-2500 nucleotides long. In some embodiments, the circRNA is about 2000 nucleotides long. In some embodiments, the circRNA is about 2100 nucleotides long. In some embodiments, the circRNA is about 2200 nucleotides long. In some embodiments, the circRNA is about 2300 nucleotides long. In some embodiments, the circRNA is about 2330 nucleotides long. In some embodiments, the circRNA is 2332 nucleotides long. In some embodiments, the circRNA is about 2500 nucleotides long. In some embodiments, the circRNA is about 2400 nucleotides long. In some embodiments, the circRNA is about 2500 nucleotides long.

FIG. 1B shows a schematic of a circRNA drug product of the present disclosure comprising a circRNA encoding for, for example, an anti-CD19 CAR, encapsulated by a lipid nanoparticle (LNP) conjugated to a targeting moiety. The circRNA encapsulated in a targeted LNP (“RNA-LNP”) is precisely designed for targeted delivery to T cells. In some embodiments, the LNP comprises an exemplary ionizable lipid (e.g., Compound 7), and the LNP is conjugated to an exemplary targeting moiety, e.g., anti-CD5 VHH, for robust targeted delivery to T cells, e.g., CD4⁺ and CD8⁺ T-cells. In some embodiments, the LNP comprises five lipid components: an ionizable lipid (e.g., Compound 7); a phospholipid, e.g., DSPC (1,2-distearoyl-sn-glycero-3-phosphocholine); a sterol lipid, e.g., cholesterol; and two PEG-lipids, e.g., DMG-PEG 2000 (1,2-dimyristoyl-rac-glycero-3-methoxypolyethylene glycol-2000), and DMG-PEG 2000-maleimide (1,2-distearoyl-sn-glycero-3-phosphoethanolamine-N-[maleimide(polyethylene glycol)-2000]). In some embodiments, LNP includes the ionizable lipid (e.g., Compound 7) at 30 to 49 mol %; the phospholipid (e.g., DSPC) at 5 to 40 mol %; the sterol lipid (e.g., cholesterol) at 15 to 50 mol %; and the two PEG-lipids, e.g., DMG-PEG 2000 and DMG-PEG 2000-maleimide (1,2-distearoyl-sn-glycero-3-phosphoethanolamine-N-[maleimide(polyethylene glycol)-2000]. In some embodiments, the five aforementioned lipid components are at a ratio of 39: 20: 39: 1.9 0.1 mol %. In some embodiments, the five aforementioned lipid components are at a ratio of 39: 20: 39: 1.9: 0.1 mol %. In some embodiments, the VHH component is a nanobody, consisting of approximately 129 amino acids, designed to bind to the human CD5 receptor. In some embodiments, the VHH nanobody is specifically engineered to attach a conjugation handle at its C terminal position, for example, to provide a cysteine residue within the conjugation handle, allowing for site-specific conjugation to the LNP via exemplary thiol/maleimide chemical conjugation. In some embodiments, the VHH that is conjugated to the LNP retains its binding affinity and specificity, enabling efficient interaction between the LNP and intended cellular target.

FIG. 1C is a schematic that shows an exemplary mechanism of action of the circRNA drug product without wishing to be bound by any particular theory. Although not bound by any mechanism, the targeting moiety, e.g., anti-CD5 nanobody, allows specific targeting of the circRNA encapsulated in the LNP to the targeting receptor, e.g., T cells that express CD5. Thus, the circRNA encoding the anti-CD19 CAR is delivered to the T cell, which uses the T cell's own machinery to translate the anti-CD19 CAR and express the CAR on the surface of the T cell. The CAR T cell then binds to its target, e.g., B cells expressing CD19, via the antigen-binding domain of the CAR. Antigen binding to the CAR triggers intracellular signaling domains, leading to T cell activation that induces secretion of cytokines and cytotoxic molecules. These molecules destroy and eliminate target B cells, resulting in B cell depletion, which is the intended on-target effect for the therapeutic treatment of B cell-mediated diseases.

### Example 2. Exemplary IRES Sequences in circRNA Constructs Improved Reporter Gene Expression and CAR Expression in T Cells

A. Effect of circRNA Comprising Various IRES, Including Exemplary IRES, on In Vitro Reporter Expression in Primary T Cells

This example illustrates that exemplary circRNA (encoding the fluorescent reporter mGreenLantern) with specific IRESs showed high reporter expression in vitro in activated primary human T cells. The exemplary circRNAs described herein was the result of a large screening campaign of over 850 IRESs, which was further down-selected for small-scale testing in activated primary human T cells.

In this example, mGreenLantern expression was evaluated in T cells transfected with circRNAs comprising various IRES sequences, including those described in Table 15A. T cells were activated and electroporated with circRNA. Post-transfection, mGreenLantern expression was measured via a live-cell imaging system (Incucyte) for four days.

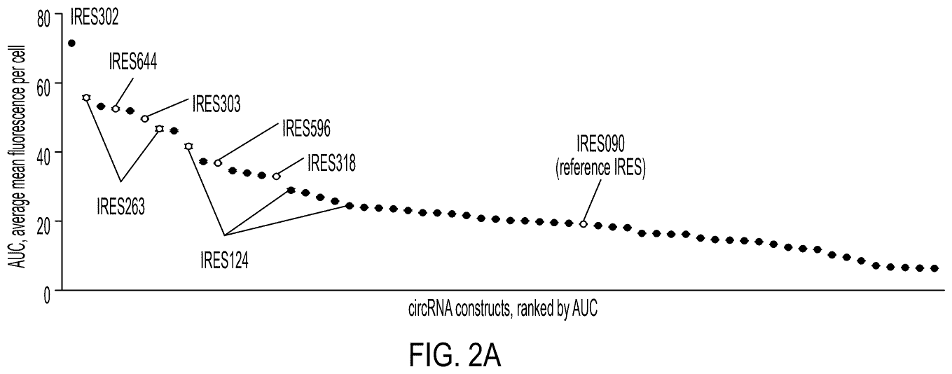

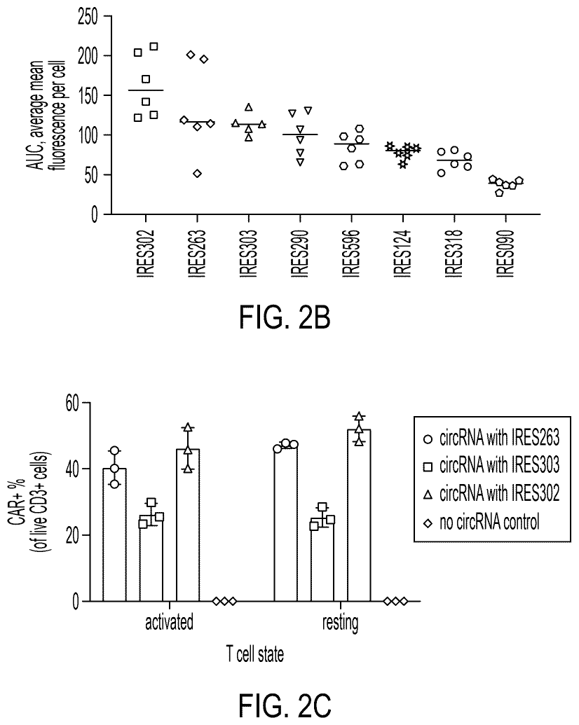

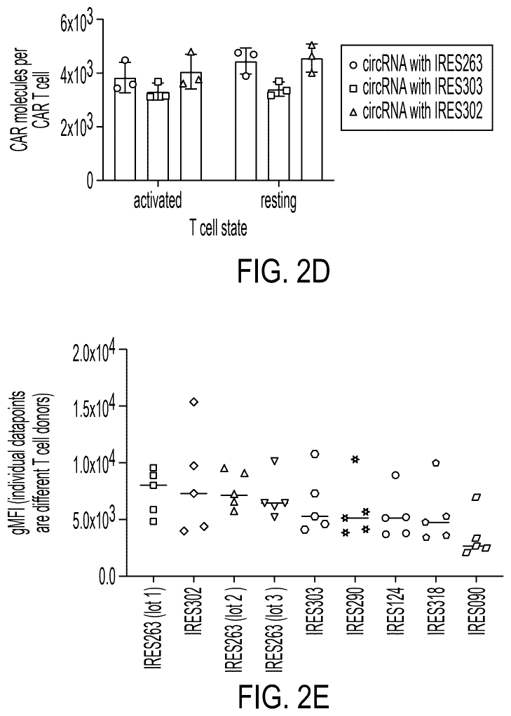

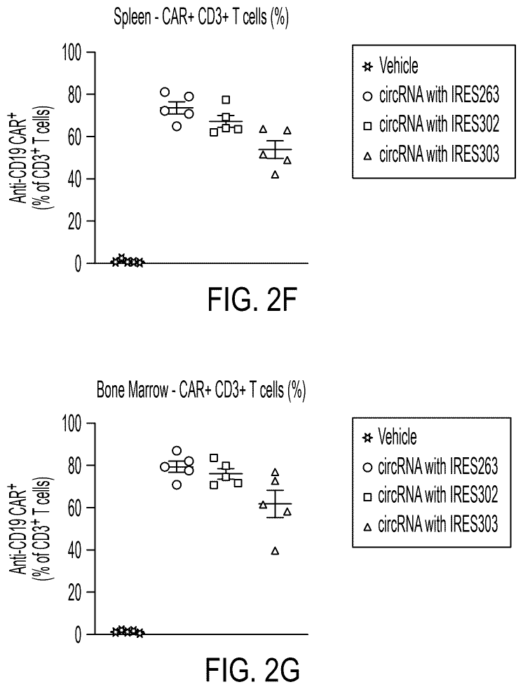

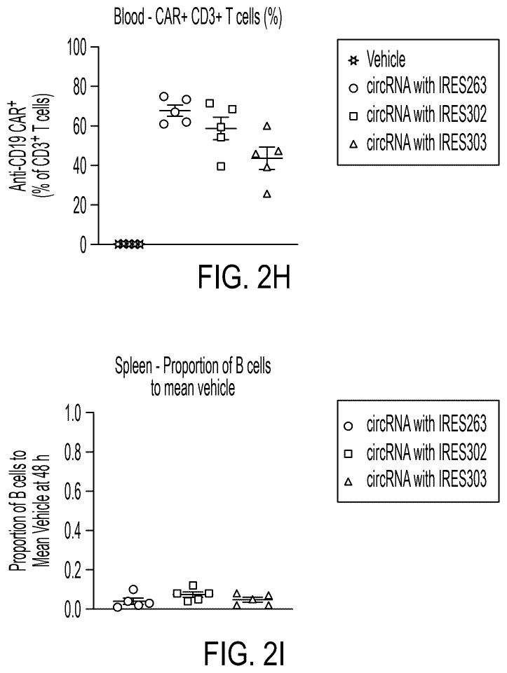

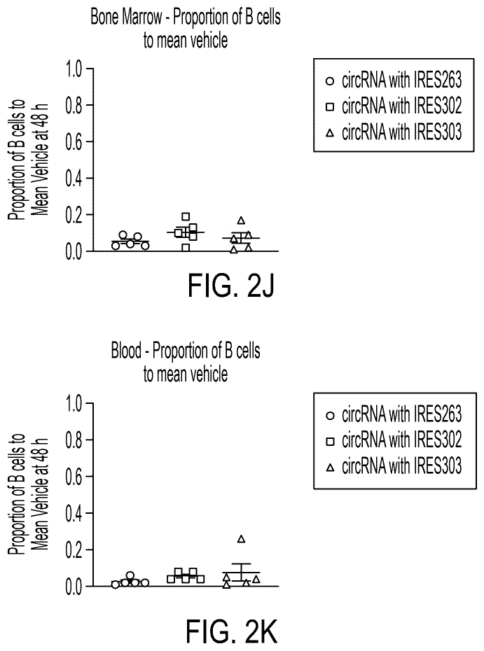

FIG. 2A is a plot of area under the curve (AUC) for mGreenLantern expression from circRNA constructs comprising multiple exemplary IRESs, with each point representing a circRNA construct. A panel of 46 IRESs was screened in a single T cell donor. Following T-cell activation, circRNAs were electroporated into cells at a dose of 125 ng RNA/5×10⁵ cells, and mGreenLantern expression was monitored via Incucyte. A majority of circRNAs comprising the exemplary IRESs (35/46 IRESs) showed expression greater than that of the reference control IRES090, which had an AUC of approximately 19 mean fluorescence units of expression. In FIG. 2A, several high-performing IRESs and the reference IRES are specifically labeled. For some IRESs, different lots of circRNA constructs comprising the same IRES was run; thus, in some instances, multiple points represent the same IRES. Table 15A lists IRESs that showed higher expression than the reference IRES for mGreenLantern expression in the IRES screen shown in FIG. 2A.

TABLE 15A
Exemplary IRES sequences and their performance in
reporter assay shown in FIG. 2A

| | AUC value(s) of mGreenLantern expression (+/− |
|---|---|
| IRES | Standard Error of the Mean, SEM) |
| IRES302 | 71.5 (±0.56) |
| IRES263 | 55.74 (±0.54), 46.76 (±0.65) |
| IRES642 | 53.21 (±0.16) |
| IRES644 | 52.52 (±0.27) |
| IRES653 | 51.92 (±0.15) |
| IRES303 | 49.63 (±0.23) |
| IRES647 | 46.15 (±0.24) |
| IRES124 | 41.67 (±0.59), 28.97 (±0.47), 24.43 (±0.2) |
| IRES147 | 37.26 (±0.38) |
| IRES596 | 36.8 (±0.18) |
| IRES411 | 34.64 (±0.27), 33.23 (±0.19) |
| IRES160 | 33.95 (±0.16) |
| IRES318 | 32.96 (±0.18) |
| IRES217 | 28.22 (±0.15) |
| IRES288 | 26.91 (±0.16) |
| IRES655 | 25.78 (±0.12) |
| IRES726 | 24 (±0.062) |
| IRES639 | 23.8 (±0.069) |
| IRES307 | 23.59 (±0.084) |
| IRES389 | 23.1 (±0.12), 22.45 (±0.11) |
| IRES325 | 22.38 (±0.064) |
| IRES327 | 22.13 (±0.062) |
| IRES659 | 21.63 (±0.068) |
| IRES693 | 20.81 (±0.049) |
| IRES772 | 20.62 (±0.16) |
| IRES537 | 20.18 (±0.085) |
| IRES703 | 20.09 (±0.038) |
| IRES758 | 19.85 (±0.11) |
| IRES753 | 19.6 (±0.085) |
| IRES650 | 19.41 (±0.11) |
| IRES090 | 19.13 (±0.14) |

FIG. 2B is a plot showing area under the curve (AUC) for reporter expression by circRNAs comprising exemplary and reference IRES in five T-cell donors, including technical duplicate measurements of one donor (Donor-080). For a given IRES, each point indicates the AUC value for one T-cell donor, and the horizontal line represents the median AUC value across all donors. Following T-cell activation, circRNAs encoding the exemplary IRES were electroporated into cells at a dose of 350 ng/1×10⁶ cells, and mGreenLantern expression was monitored via Incucyte for four days. As shown in FIG. 2B, all circRNAs comprising the exemplary IRESs showed an AUC greater than that of the reference control in all T-cell donors tested.

In summary, the results shown in FIGS. 2A and 2B demonstrated that optimizing IRESs in the circRNA construct improved reporter gene expression in activated T cells.

### B. CircRNAs Comprising Exemplary IRESs Showed High CAR Expression In Vitro in Resting and Activated T Cells

This example illustrates that circRNA (encoding anti-CD19 CAR) comprising exemplary IRESs showed high anti-CD19 CAR expression in vitro in both resting and activated T cells.

In this example, anti-CD19 CAR expression of the RNA-LNP constructs was evaluated in resting and activated T cells. The circRNA constructs comprised exemplary IRESs and encoded exemplary anti-CD19 CARs as described in Table 15B, and were encapsulated in LNP comprising Compound 7 with an anti-CD5 targeting moiety (VHH319). The RNA-LNP constructs were transfected into resting and activated T cells (150 ng RNA/1×10⁵ cells). The “no circRNA control” cells were treated with PBS buffer instead of with LNP. CAR expression was measured at day 2 post-transfection by fluorescence-activated cell sorting (FACS).

TABLE 15B
CircRNA construct encoding exemplary anti-CD19 CAR
with corresponding IRES within the circRNA construct

| | Exemplary | IRES in the |
|---|---|---|
| | anti-CD19 | circRNA |
| | CAR | construct |
| CircRNA encoding anti-CD19 | SEQ ID | SEQ ID NO: |
| CAR (SEQ ID NO: 125) with | NO: 125 | 1; IRES263 |
| IRES263 (“CircRNA with | | |
| IRES263”) | | |
| CircRNA encoding anti-CD19 | SEQ ID | SEQ ID NO: |
| CAR (SEQ ID NO: 215) with | NO: 215 | 3; IRES303 |
| IRES303 | | |
| (“CircRNA with IRES303”) | | |
| CircRNA encoding anti-CD19 | SEQ ID | SEQ ID NO: |
| CAR (SEQ ID NO: 214) with | NO: 214 | 2; IRES302 |
| IRES302 | | |
| (“CircRNA with IRES302”) | | |

FIG. 2C is a graph of percentage of CAR-positive activated and resting T cells, relative to the total number of live CD3-positive cells, 2 days post-transfection. The results shown in FIG. 2C indicated that approximately 20% to 55% of T cells (both resting and activated T cells) expressed anti-CD19 CAR encoded by circRNAs containing the exemplary IRES sequences.

FIG. 2D is a graph of total CAR molecules per CAR T cell in resting and activated T cells. As shown in FIG. 2D, between 3000 and 5000 CAR molecules were expressed per CAR T cell.

The results in FIG. 2C and FIG. 2D showed that circRNAs comprising the exemplary IRES drove high CAR expression in transfected resting and activated T cells in vitro.

In summary, this example showed that the exemplary RNA-LNPs with circRNAs encoding anti-CD19 CAR and comprising the IRES sequences described in this Example and herein (e.g., Table 16) showed high CAR expression in vitro in resting and activated T cells.

C. Effect of circRNA Constructs with Exemplary IRESs on CAR Expression In Vitro in Activated T Cells from Multiple Donors

This example illustrates that exemplary circRNAs (encoding for anti-CD19 CAR) with different IRESs showed high CAR expression in T cells from several donors.

The circRNA constructs comprised exemplary IRESs and encoded exemplary anti-CD19 CARs as described in Table 15B, and were encapsulated in LNP comprising Compound 7 with an anti-CD5 targeting moiety (VHH319).

T cells from five donors were activated and electroporated with 100 ng-200 ng RNA/1×10⁶ cells. Post-transfection, CAR expression was measured by fluorescence activated cell sorting (FACS) on days 1 and 4. FIG. 2E shows CAR expression (gMFI) on day 1. Each point represents the CAR expression for a single donor. Median expression for each circRNA is represented by a horizontal line. For circRNA comprising IRES 263 (SEQ ID NO: 1), multiple RNA lots were transfected into T cells in parallel.

This example showed that circRNA constructs comprising exemplary IRESs showed high anti-CD19 CAR expression in activated T cells from multiple donors, which indicated that CAR expression was not donor-specific. Further, this example showed that circRNA comprising all IRES tested increased CAR expression compared to the reference IRES (IRES090).

D. Effect of circRNA Comprising Exemplary IRESs on CAR Expression in T Cells in Spleen In Vivo

This example illustrates that circRNA encoding exemplary anti-CD19 CAR comprising specific IRESs showed high CAR expression in T cells in vivo (in the spleen, blood, and bone marrow of humanized mice).

In this example, anti-CD19 CAR expression was evaluated in T cells following administration of exemplary RNA-LNPs. The circRNA constructs comprised exemplary IRESs and encoded exemplary anti-CD19 CARs as described in Table 15C, and were encapsulated in LNP comprising Compound 7 with an anti-CD5 VHH319 targeting moiety. As shown in Table 15C, the circRNAs tested (encoding for the exemplary anti-CD19 CAR) comprised the IRES designated as IRES263 (SEQ ID NO: 1), IRES302 (SEQ ID NO: 2), or IRES303 (SEQ ID NO: 3). The study also included a “Vehicle” control, in which the mice was treated with phosphate-buffered saline (PBS) buffer.

TABLE 15C
Exemplary anti-CD19 CAR and corresponding IRES

| | Exemplary | IRES in the |
|---|---|---|
| | anti-CD19 | circRNA |
| | CAR | construct |
| CircRNA encoding anti- | SEQ ID | SEQ ID NO: |
| CD19 CAR (SEQ ID NO: | NO: 125 | 1; IRES263 |
| 125) with IRES263 | | |
| (“CircRNA with | | |
| IRES263”) | | |
| CircRNA encoding anti- | SEQ ID | SEQ ID NO: |
| CD19 CAR (SEQ ID NO: | NO: 214 | 2; IRES302 |
| 214) with IRES302 | | |
| (“CircRNA with | | |
| IRES302”) | | |
| CircRNA encoding anti- | SEQ ID | SEQ ID NO: |
| CD19 CAR (SEQ ID NO: | NO: 215 | 3; IRES303 |
| 215) with IRES303 | | |
| (“CircRNA with | | |
| IRES303”) | | |

The RNA-LNPs and “Vehicle” control were delivered in hPBMC-engrafted NSG (humanized) mice. CAR expression and percent B cells remaining 48 hours post-LNP administration were measured by fluorescence-activated cell sorting (FACS).

FIG. 2F is a graph of percentage of anti-CD19 CAR-positive cells in vivo in spleen as a percentage of total CD3+ T cells. The results shown in FIG. 2F indicated that approximately 40%-80% of splenic T cells expressed anti-CD19 CAR. By contrast, the vehicle showed no detectable level of CAR expression. Delivery of the circRNA comprising the IRES of SEQ ID NO: 1 resulted in the highest CAR expression in 60%-80% of CD3+ T cells.

FIG. 2G is a graph of in vivo anti-CD19 CAR expression in bone marrow as a percentage of total CD3+ T cells for various RNA-LNPs comprising the exemplary IRESs tested.

FIG. 2H is a graph of in vivo anti-CD19 CAR expression in blood as a percentage of total CD3+ T cells for various RNA-LNPs comprising the exemplary IRESs tested.

The results in FIG. 2G and FIG. 2H showed that high CAR expression (relative to a vehicle control) was also observed in bone marrow and blood of the humanized mice, in which 60%-80% of CD3+ T cells and 40%-80% of CD3+ T cells were anti-CD19 CAR+, respectively.

FIG. 2I is a graph of the proportion of B cells remaining in spleen relative to a vehicle control for various RNA-LNPs comprising the exemplary IRESs tested. The results in FIG. 2I showed that 3.9% B cells remained with the circRNA construct comprising IRES263 (SEQ ID NO: 1), 7.4% B cells remained with the circRNA construct comprising IRES302 (SEQ ID NO: 2), and 4.7% B cells remained with the circRNA construct comprising IRES303 (SEQ ID NO: 3).

FIG. 2J is a graph of proportion of B cells remaining in bone marrow relative to a vehicle control for various RNA-LNPs comprising the exemplary IRESs tested. The results in FIG. 2J showed 5.2% B cells remained with the circRNA construct comprising IRES263 (SEQ ID NO: 1), 10.4% B cells remained with the circRNA construct comprising IRES302 (SEQ ID NO: 2), and 7.3% B cells remained with the circRNA construct comprising IRES303 (SEQ ID NO: 3).

FIG. 2K is a graph of proportion of B cells remaining in blood relative to a vehicle control for various RNA-LNPs comprising the exemplary IRESs tested. The results in FIG. 2K showed that only 2.4% B cells remained with the circRNA construct comprising IRES263 (SEQ ID NO: 1), 5.4% B cells remained with the circRNA construct comprising IRES302 (SEQ ID NO: 2), and 7.6% B cells remained with the circRNA construct comprising IRES303 (SEQ ID NO: 3).

In summary, this study showed that exemplary RNA-LNPs comprising circRNAs encoding for anti-CD19 CAR and comprising three exemplary IRES sequences showed robust in vivo CAR expression in T cells in the spleen, bone marrow, and blood of humanized mice. Anti-CD19 CAR expression in T cells resulted in substantial depletion of B cells in the spleen, blood, and bone marrow of humanized mice.

### Example 3. Optimization of Spacers in the Untranslated (UTR) Region of circRNA Enhanced Anti-CD19 CAR Expression

This example illustrates that optimization of the untranslated regions in the circRNA resulted in enhanced expression of anti-CD19 CAR coding sequence.

FIG. 3A is a schematic of circRNA designs showing that, in some embodiments, the 5′ UTR comprises a spacer, and the 3′ UTR comprises a spacer.

Briefly, exemplary 3′ UTR variants were engineered and tested for effects on eGFP (enhanced green fluorescent protein) reporter expression in HEK293T (immortalized human embryonic kidney) cells.

FIG. 3B is a graph of area under the curve of eGFP expression in HEK293T cells upon insertion of exemplary 3′ spacers in the circRNA construct. The sequences of the exemplary 3′ spacers are listed in Table 2B herein. The “linear” mRNA control has no spacer sequences. As seen from the results in FIG. 3B, including exemplary spacers in the 3′ UTR enhanced reporter gene expression in the majority of circRNA constructs.

FIG. 3C is a graph of CAR expression in human T cells from a circRNA comprising an exemplary CAA40 spacer in the 3′ UTR. Three replicates are shown. A no RNA control and a scrambled IRES (negative control) was included. The results in FIG. 3C showed comparable CAR expression in three replicate experiments with CAA40 spacer in the 3′ UTR.

As seen in FIG. 3B, engineered UTR variants in the circRNA construct showed improvements in reporter gene expression and, as seen in FIG. 3C, engineered UTR variants in the circRNA construct showed robust and consistent CAR expression in vivo in human cells.

Overall, the results from this example showed that optimizing the untranslated regions in the circRNA construct resulted in higher expression of the anti-CD19 coding sequence.

### Example 4. Inclusion of One or More miRNA Binding Sites in Untranslated Region (UTR) of the circRNA Increased Specificity of Anti-CD19 CAR with Reduced Off-Target Expression In Vitro and In Vivo

A. Effect of One or More miRNA Binding Site(s) in UTR on Anti-CD19 Expression In Vitro

This example demonstrates that including one or more miRNA binding sites in the UTR of the circRNA construct decreased off-target activity (e.g., liver expression) with minimal effects on on-target expression in vitro.

Circular RNA constructs encoding for anti-CD19 CAR was engineered with either no miRNA binding sites (SEQ ID NO: 175), one copy of miR-122_1 spacer (SEQ ID NO: 228), one copy of miR-122_2 spacer (SEQ ID NO: 264), or three copies of miR-122 spacers (SEQ ID NO: 229) in the 3′ UTR. Each of the circRNAs was encapsulated in an LNP comprising ionizable lipid Compound 7 conjugated to an anti-CD5 targeting moiety (VHH319) and delivered to primary human T cells (on-target cells at 150 ng RNA/1×10⁵ activated T cells)) and Huh7 immortalized hepatocytes (off-target cells). Anti-CD19 CAR expression was measured as both percent CAR expression in T cells (FIG. 4A) as well as geometric mean fluorescence intensity (gMFI)(FIG. 4B).

FIG. 4A is a graph of percent CAR expression in T cells from three donors (target cells) transfected with a circRNA encapsulated in a LNP comprising Compound 7, with an anti-CD5 targeting moiety (VHH319), wherein the circRNA had no miR-122 binding site (“RNA-LNP with no miR-122”), one miR-122 binding site (“RNA-LNP with 1 miR-122”), or three miR-122 binding sites (“RNA-LNP with 3 miR-122”). “TBS” indicates that cells were treated with Tris-buffered saline (TBS), and “Full control” indicates that the cells received no treatment.

FIG. 4B is a graph of CAR expression measured as gMFI in T cells from three donors (target cells) transfected with a circRNA encapsulated in a LNP comprising Compound 7, with an anti-CD5 VHH319 targeting moiety, wherein the circRNA had no miR-122 binding site (“RNA-LNP with no miR-122”), one miR-122 binding site (“RNA-LNP with 1 miR-122”), or three miR-122 binding sites (“RNA-LNP with 3 miR-122”). “TBS” indicates that cells were treated with Tris-buffered saline (TBS), and “Full control” indicates that the cells received no treatment.

FIG. 4A and FIG. 4B showed that in primary human T cells from three different donors, the circRNA constructs comprising one miRNA copy or three miRNA copies in the 3′ UTR showed marginally reduced expression but were generally comparable to the circRNA that contained no miRNA site in the 3′ UTR.

FIG. 4C is a graph of percent CAR expression in Huh7 immortalized hepatocytes (off-target liver cells) transfected with circRNA (at a dose of 150 ng RNA/1×10⁵ cells) encapsulated in a LNP comprising Compound 7, with an anti-CD5 targeting moiety (VHH319), wherein the circRNA had no miR-122 binding site (“RNA-LNP with no miR-122”), one miR-122 binding site (“RNA-LNP with 1 miR-122”), or three miR-122 binding sites (“RNA-LNP with 3 miR-122”) in the 3′ UTR.

FIG. 4D is a graph of CAR expression (gMFI) in Huh7 immortalized hepatocytes (off-target liver cells) transfected with a circRNA (at a dose of 150 ng RNA/1×10⁵ cells) encapsulated in a LNP comprising Compound 7, with an anti-CD5 targeting moiety (VHH319), wherein the circRNA had no miR-122 binding site (“RNA-LNP with no miR-122”), one miR-122 binding site (“RNA-LNP with 1 miR-122”), or three miR-122 binding sites (“RNA-LNP with 3 miR-122”) in the 3′ UTR.

FIG. 4C and FIG. 4D showed that in Huh7 immortalized hepatocytes (off-target cells), the expression of anti-CD19 CAR was at a similar scale to target cells. However, adding even a single copy of a miR-122 binding site to the 3′ UTR dramatically reduced off-target expression. Adding three copies of a miR-122 binding site to the 3′ UTR did not further decrease off-target expression.

Overall, this example illustrates that the inclusion of one or more copies of microRNA binding sites in the untranslated region of the circRNA increased specificity and decreased off-target effects in vitro.

B. Effect of One or More miRNA Binding Sites in UTR on Anti-CD19 Expression In Vivo

This example demonstrates that including one or more miRNA binding sites in the UTR decreased off-target activity with minimal effects on on-target expression in vivo.

CircRNA comprising luciferase reporter constructs was engineered with either no miRNA binding sites, one copy of miR-122 binding site, or three copies of miR-122 binding sites in the 3′ UTR. Each of the circRNAs was encapsulated in LNP comprising exemplary Compound 7 (either “Base LNP” with no targeting moiety or LNP conjugated to an anti-CD5 targeting moiety (VHH319)), and in vivo luciferase expression in T cells in spleen (on-target cells) and liver (off-target cells) was measured. The study also included a “Vehicle” control, in which a buffer was administered.

FIG. 4E is a graph of luciferase expression in spleens of CD34⁺ HSC-engrafted humanized NSG mice at 24 hours after administration of various RNA-LNPs comprising circular RNA constructs comprising one or three miR-122 binding sites, relative to circRNA constructs comprising no miR-122 binding site in the 3′ UTR. FIG. 4E showed that in vivo, the circRNA constructs comprising one miR-122 binding site or three miR-122 binding sites in the 3′ UTR showed comparable expression to circRNA constructs comprising no miR-122 binding site in the 3′ UTR.

FIG. 4F is a graph of luciferase expression in livers of CD34⁺ HSC-engrafted humanized NSG mice at 24 hours after administration of various RNA-LNPs comprising circular RNA constructs comprising one or three miR-122 binding sites, relative to circRNA constructs comprising no miR-122 binding site in the 3′ UTR. Each of the circRNAs was encapsulated in LNP comprising exemplary Compound 7 (either “Base LNP” with no targeting moiety or LNP conjugated to an anti-CD5 targeting moiety (VHH319)), and in vivo anti-CD19 expression in T cells in spleen (on-target cells) was measured. Further, circRNA encoding anti-CD19 CAR with either no miRNA sites (SEQ ID NO: 175), or one copy of miR-122 binding site (SEQ ID NO: 264) in the 3′ UTR were compared.

FIG. 4F showed that in the liver (off-target cells), the expression of anti-CD19 CAR with an anti-CD5 targeting moiety (VHH319) was comparable to non-targeting LNPs; in other words, off-target effects were high. However, adding even a single copy of a miR-122 binding site to the 3′ UTR of the circRNA construct dramatically reduced off-target expression. Adding three miR-122 binding sites further decreased off-target expression only marginally over one miR-122 binding site.

The results in FIG. 4F also showed that there was comparable CAR expression in target cells with or without a single miRNA site. In other words, including a miRNA site resulted in no loss of expression relative to a circRNA construct having no miRNA sites.

Overall, this example illustrates that the inclusion of one or more microRNA binding site(s) in the untranslated region of the circRNA increased specificity, and decreased off-target effects in vivo without affecting on-target expression.

C. Effect of Redesigned miR-122_2 Spacer Relative to miR-122_1 Spacer in UTR of the circRNA Construct on Anti-CD19 Expression In Vivo

The miR-122_1 spacer in the UTR of the circRNA construct was redesigned to interrupt greater than five poly A tracts in the miR-122_2 spacer by replacing the adenines (A) with cytosines (C) in the poly A tracts of the sequences that flank the miR122 binding site.

FIG. 4G shows a schematic of the designs of the miR-122_1 spacer and miR-122_2 spacer in the UTR of the circRNA constructs with re-designed flanking sequences. In other embodiments, a similar strategy of replacing As with Cs in the poly A tract of sequences that flank the microRNA binding site can be performed to redesign other UTRs in the circRNA construct.

FIG. 4H is a graph of percent circularization of in vitro transcribed RNA. As shown in FIG. 4H, inclusion of either miR-122_1 spacer (within SEQ ID NO: 228) or miR-122_2 spacer (within SEQ ID NO: 264) showed comparable circularization, with an improvement in circularization (8.2% increase from 51.4% to 59.6% circularization) with inclusion of the miR-122_2 spacer in the 3′ UTR. Overall, these results showed that interrupting poly A tracts with cytosine resulted in some improvement in RNA circularization.

FIG. 4I is a graph of hepatic cell knockdown by circRNA comprising UTR with a miR-122_2 spacer (miR-122_2 binding site and flanking regions) relative to miR-122_1 spacer (miR-122_1 binding site and flanking regions). In Huh7 hepatocytes as measured by flow cytometry, 24 hours after transfection, the percent CAR expression was comparably decreased by inclusion of either miR-122_1 spacer or miR-122_2 spacer relative to no miR-122 binding site in the 3′ UTR of the circRNA construct. “Full control” refers to a no treatment control (with no buffer or reagent addition, no RNA, and no electroporation). “EP Only” refers to an empty electroporation control where cells were electroporated in the absence of RNA).

FIG. 4J is a graph of hepatic cell knockdown by circRNA with the miR-122_2 (SEQ ID NO: 267) spacer relative to miR-122_1 (SEQ ID NO: 270) spacer. In Huh7 hepatocytes as measured by flow cytometry, 24 hours after transfection, CAR gMFI levels were comparably decreased by inclusion of either miR-122_1 spacer or miR-122_2 spacer relative to no miR-122 binding site in the 3′ UTR of the circRNA construct. In both FIG. 4I and FIG. 4J, “Full Control” indicates cells that received no treatment, and “EP only” indicates cells that were subjected to electroporation, but no RNA was present.

FIG. 4K is a graph of anti-CD19 CAR positive cells as a percentage of CD3⁺ T cells from spleens of CD34⁺ HSC-engrafted humanized NSG mice at 24 hours after administration of various circRNA constructs encapsulated in LNP. This data compares an exemplary circRNA construct without a miRNA binding site (SEQ ID NO: 175) and a circRNA construct with one miRNA-binding site (SEQ ID NO: 264), which contained a single miR-122 binding site in 3′ UTR.

FIG. 4L is a graph of anti-CD19 CAR expression in blood as a percentage of total CD3+ T cells following administration of circRNA including no miRNA binding site (SEQ ID NO: 175 or one miR-122_2 spacer in the 3′ UTR (SEQ ID NO: 264). The circRNAs were encapsulated in LNPs conjugated to an anti-CD5 targeting moiety (VHH319) and were formulated in two exemplary formulations, LNP-11 and LNP-15 (as described in Table 16).

FIG. 4M is a graph of proportion of B cells relative to vehicle control in blood following administration of circRNA including no miRNA binding site (SEQ ID NO: 175) or one miR-122_2 binding site in the 3′ UTR (SEQ ID NO: 264). The circRNAs were encapsulated in LNPs conjugated to an anti-CD5 targeting moiety (VHH319) and were formulated in two exemplary formulations LNP-11 and LNP-15 (as described in Table 16).

FIG. 4N is a graph of anti-CD19 CAR expression in spleen as a percentage of total CD3+ T cells following administration of circRNA including no miR-122_2 spacer (SEQ ID NO: 175) or one miR-122_2 spacer in the 3′ UTR (SEQ ID NO: 264). The circRNAs were encapsulated in LNPs conjugated to an anti-CD5 targeting moiety (VHH319) and were formulated in two exemplary formulations LNP-11 and LNP-15 (as described in Table 16).

FIG. 4O is a graph of proportion of B cells relative to vehicle control in the spleen following administration of circRNA including no miRNA binding site (SEQ ID NO: 175) or one miR-122_2 binding site in the 3′ UTR (SEQ ID NO: 264). The circRNAs were encapsulated in LNPs conjugated to an anti-CD5 targeting moiety (VHH319) and were formulated in two exemplary formulations LNP-11 and LNP-15 (as described in Table 16).

Overall, the results showed that circRNA constructs comprising UTRs comprising the miR-122_2 and miR-122_1 spacers were functionally comparable with respect to CAR expression and B cell depletion in the blood and spleen. Circularization was slightly improved with a circRNA construct comprising UTRs including the miR-122_2 spacer compared to the miR-122_1 spacer.

D. Hydropathy Index of a Redesigned miR-122_2 Upstream and Downstream Spacer Relative to miR-122_1 Upstream and Downstream Spacer

In this example, a hydropathy index is calculated for the miR-122_2 and miR-122_1 upstream and downstream spacers. As described herein, hydropathy index is a quantitative representation of the hydrophobicity and hydrophilicity of an RNA sequence by combining experimentally measured nucleobase partition coefficients (log P) in an immiscible organic solvent (e.g., n-Octanol) and an aqueous solvent (e.g., water), and subsequently normalizing by length of RNA sequence. A more negative log P is indicative of a more hydrophilic sequence and a less negative log P is indicative of a more hydrophobic sequence.

Analysis by Protocol for Assigning a Residue's Character on the Hydropathy (PARCH) scale has revealed that base composition is the primary determinant of sequence hydropathy as the phosphate-ribose backbone is uniformly highly hydrophilic and does not vary between the sequences being compared. The n-Octanol/water log P values for canonical nucleobases have been established experimentally (Zamora et al., 2017), and are shown below.

Experimental nucleobase log P values (Zamora et al., 2017)

| | Experimental log P | |
|---|---|---|
| Base | (n-Octanol/water) | Relative Hydropathy |
| Cytosine (C) | −1.7 | Most Hydrophilic |
| Uracil (U) | −1.1 | Moderate Hydrophilicity |
| Guanine (G) | −0.9 | Moderate Hydrophilicity |
| Adenine (A) | −0.1 | Least Hydrophilic |

In this Example, hydropathy index was calculated as follows: (i) the miR-122_1 and miR-122_2 upstream and downstream RNA sequences were analyzed (thymines (Ts) rendered as uracils (Us) for RNA sequences); (ii) the number of nucleobases nA, nC, nG, nU were counted; (iii) additive hydropathy sum was calculated (Hraw) by the formula: H_{raw}=Σᵢ(nᵢ*log Pᵢ), and lastly (iv) hydropathy index was calculated by normalizing to sequence length (L):

$$Hydropathy Index = \frac{{H}_{r a w}}{L} .$$

Accordingly, the intrinsic hydropathy of the primary sequence was calculated without consideration for RNA folding, secondary structure, or tertiary interactions (such as base stacking or duplex formation). Normalizing to length provides length-independent hydropathy indices that can be used to compare RNAs of any size, and also provides a measure of hydrophilicity and hydrophobicity per nucleotide.

Provided herein the miR-122 binding site is flanked by the miR-122_2 upstream spacer and the miR-122_2 downstream spacer as follows: aacaaacacaaacCAAACACCATTGTCACACTCCAaacaacaaacaaaca (SEQ ID NO: 267)

miR122_2 upstream spacer: aacaaacacaaac (SEQ ID NO: 373) has a length of 13 nucleobases with the following nucleobase composition: 9 adenines, 4 cytosines, no guanines and no uracils. Accordingly,

$${H}_{r a w} = ( 9 * - 0.1 ) + ( 4 * - 1.7 ) + ( 0 * - 0.9 ) + ( 0 * - 1.1 )$$

$${H}_{r a w} = ( - 0.9 ) + ( - 6.8 ) + ( 0 ) + ( 0 ) = - 7.7$$

$$Hydropathy Index = {H}_{r a w} / L : - 7.7 / 13 = - 0.5923$$

miR122_2 downstream spacer: aacaacaaacaaaca (SEQ ID NO: 374) has a length of 15 nucleobases with the following nucleobase composition: 11 adenines, 4 cytosines, no guanines and no uracils. Accordingly,

$${H}_{r a w} = ( 11 * - 0.1 ) + ( 4 * - 1.7 ) + ( 0 * - 0.9 ) + ( 0 * - 1.1 )$$

$${H}_{r a w} = ( - 1.1 ) + ( - 6.8 ) + ( 0 ) + ( 0 ) = - 7.9$$

$$Hydropathy Index = {H}_{r a w} / L : - 7.9 / 15 = - 0.53$$

The miR-122 binding site is flanked by the miR-122_1 upstream and the miR-122_1 downstream spacer as follows: aaaaacacaaacCAAACACCATTGTCACACTCCAaaaaaaacaaaca (SEQ ID NO: 270)

miR122_1 upstream spacer: aaaaacacaaac (SEQ ID NO: 391) has a length of 12 nucleobases with the following nucleobase composition: 9 adenines, 3 cytosines, no guanines and no uracils. Accordingly,

$${H}_{r a w} = ( 9 * - 0.1 ) + ( 3 * - 1.7 ) + ( 0 * - 0.9 ) + ( 0 * - 1.1 )$$

$${H}_{r a w} = ( - 0.9 ) + ( - 5.1 ) + ( 0 ) + ( 0 ) = - 6$$

$$Hydropathy Index = {H}_{r a w} / L : - 6 / 12 = - 0.5$$

miR122_1 downstream spacer: aaaaaaacaaaca (SEQ ID NO: 392) has a length of 13 nucleobases with the following nucleobase composition: 11 adenines, 2 cytosines, no guanines and no uracils. Accordingly,

$${H}_{r a w} = ( 11 * - 0.1 ) + ( 2 * - 1.7 ) + ( 0 * - 0.9 ) + ( 0 * - 1.1 )$$

$${H}_{r a w} = ( - 1.1 ) + ( - 3.4 ) + ( 0 ) + ( 0 ) = - 4.5$$

$$Hydropathy index = {H}_{r a w} / L \text{:-} 4.5 / 13 = - 0.3462$$

### A 15 Nucleotide Poly A Spacer

15A: aaaaaaaaaaaaaaa (SEQ ID NO: 393) has a length of 15 nucleobases with the nucleobase composition of 15 adenines. Accordingly,

$${H}_{r a w} = ( 15 * - 0.1 ) + ( 0 * - 1.7 ) + ( 0 * - 0.9 ) + ( 0 * - 1.1 )$$

$${H}_{r a w} = ( - 1.5 ) + ( 0 ) + ( 0 ) + ( 0 ) = - 1.5$$

$$Hydropathy index = {H}_{r a w} / L \text{:-} 1.5 / 15 = - 0.1$$

### A 15 Nucleotide Poly C Spacer

15C: ccccccccccccccc (SEQ ID NO: 394) has a length of 15 nucleotides with the nucleobase composition of 15 cytosines. Accordingly,

$${H}_{r a w} = ( 0 * - 0.1 ) + ( 15 * - 1.7 ) + ( 0 * - 0.9 ) + ( 0 * - 1.1 )$$

$${H}_{r a w} = ( 0 ) + ( - 25.5 ) + ( 0 ) + ( 0 ) = - 2 5.5$$

$$Hydropathy index = {H}_{r a w} / L \text{:-} 25.5 / 15 = - 1.7$$

A 16 Nucleotide Space with Alternate Cytosines and Adenines

16CA: cacacacacacacaca (SEQ ID NO: 395) has a length of 16 nucleotides with the nucleobase composition of 8 adenines and 8 cytosines with no uracils and no guanines. Accordingly,

$${H}_{r a w} = ( 8 * - 0.1 ) + ( 8 * - 1.7 ) + ( 0 * - 0.9 ) + ( 0 * - 1.1 )$$

$${H}_{r a w} = ( - 0.8 ) + ( - 13.6 ) + ( 0 ) + ( 0 ) = - 1 4.4$$

$$Hydropathy index = {H}_{r a w} / L \text{:-} 14.4 / 16 = - 0.9$$

This example showed that the miR-122_2 upstream spacer has a calculated hydropathy index of −0.59. The miR-122_2 downstream spacer has a calculated hydropathy index of −0.53. The miR-122_1 upstream spacer has a calculated hydropathy index of −0.5. The miR-122_1 downstream spacer has a calculated hydropathy index of −0.346. In contrast, a 15 nucleotide poly A sequence of consecutive adenines has a calculated hydropathy index of −0.1, while a 15 nucleotide poly C sequence of consecutive cytosines has a calculated hydropathy index of −1.7. A 16 nucleotide sequence of alternating cytosines and adenines has a calculated hydropathy index of −0.9.

The miR-122_2 and miR-122_1 upstream spacers had comparable hydropathy indices. Redesigning the miR-122_2 downstream spacer made it more hydrophilic as compared to the miR-122_1 downstream spacer. Importantly, both miR-122_1 and miR-122_2 spacers were significantly more hydrophilic as compared to a poly A sequence of 15 consecutive adenines, and significantly less hydrophilic as compared to a poly C sequence of 15 consecutive cytosines or a polyAC sequence of 16 nucleobases comprising alternating cytosines and adenines.

In conclusion, this example demonstrates that optimizing spacer design engineers a favorable hydropathy index such that the circRNA is sufficiently hydrophilic that it limits undesirable interaction between the circRNA and the lipid nanoparticle, facilitating release of RNA into the target cell.

### Example 5. Exemplary Codon Optimized Coding Sequences Demonstrated CAR Expression from circRNA

In this example, exemplary codon optimized coding sequences were tested in vitro in activated T cells for anti-CD19 CAR expression by plotting number of CAR molecules per CAR T cell. Briefly, T cells were transfected with exemplary coding sequences, and anti-CD19 CAR expression was subsequently measured.

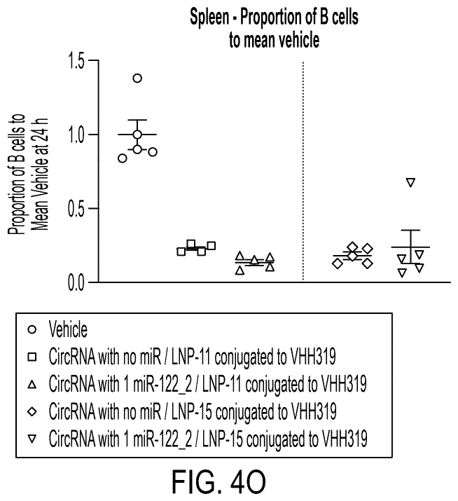

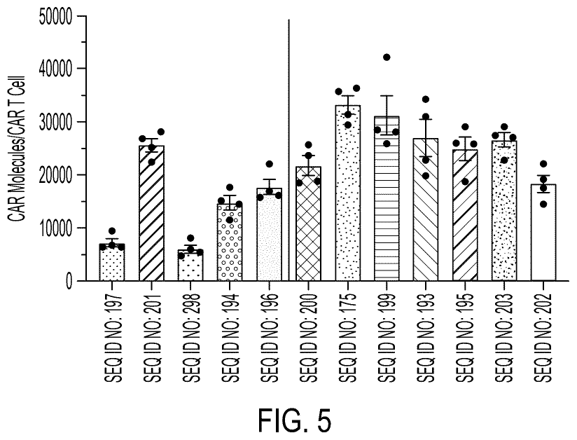

FIG. 5 is a graph of CAR expression measured by flow cytometry showing that various exemplary circRNA encoded by codon-optimized coding sequences demonstrated CAR expression in activated T cells in vitro. Specifically, the full length circRNA sequences (comprising codon-optimized coding sequences) tested were SEQ ID NO: 197, SEQ ID NO: 201, SEQ ID NO: 198 SEQ ID NO: 194, SEQ ID NO: 196, SEQ ID NO: 200, SEQ ID NO: 175, SEQ ID NO: 199, SEQ ID NO: 193, SEQ ID NO: 195, SEQ ID NO: 203, and SEQ ID NO: 202.

Overall, this example demonstrated that circRNAs comprising various exemplary codon-optimized coding sequences resulted in anti-CD19 CAR expression in vitro.

### Example 6: In Vivo CAR Expression and B Cell Depletion by circRNA Comprising Exemplary Anti-CD19 CAR Constructs with Various CAR Architectures in T Cells in the Blood and Spleen of Humanized Mice

This example demonstrated that circRNA (encoding various exemplary CAR architectures) conjugated to targeted LNP were efficacious in expressing anti-CD19 CAR and depleting B cells in the blood and spleen of humanized mice.

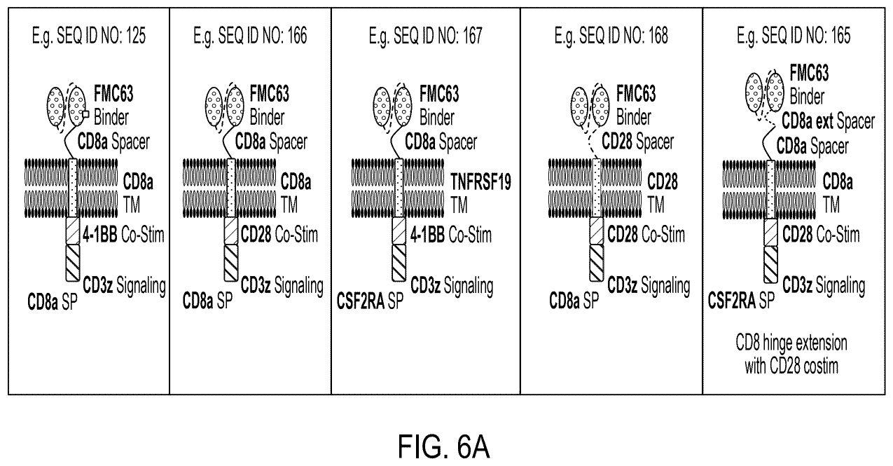

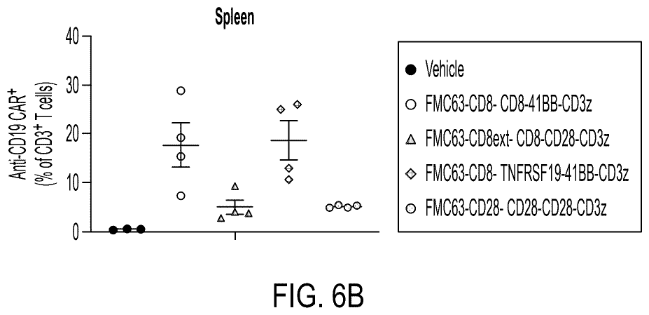

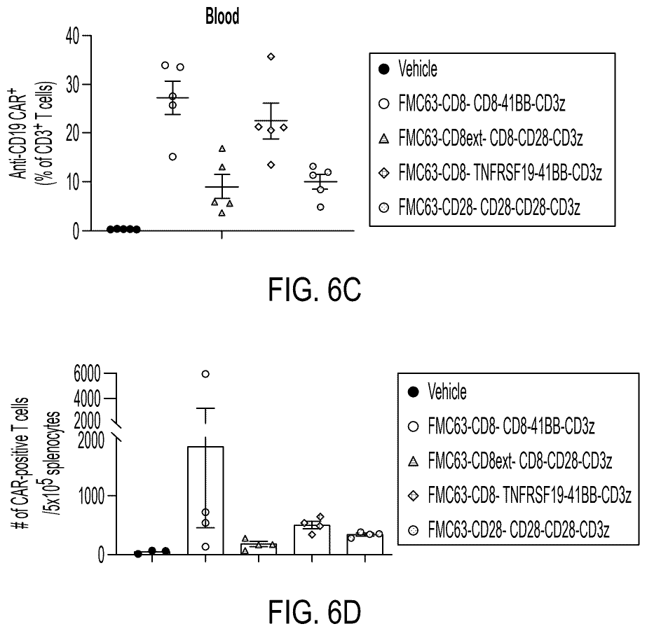

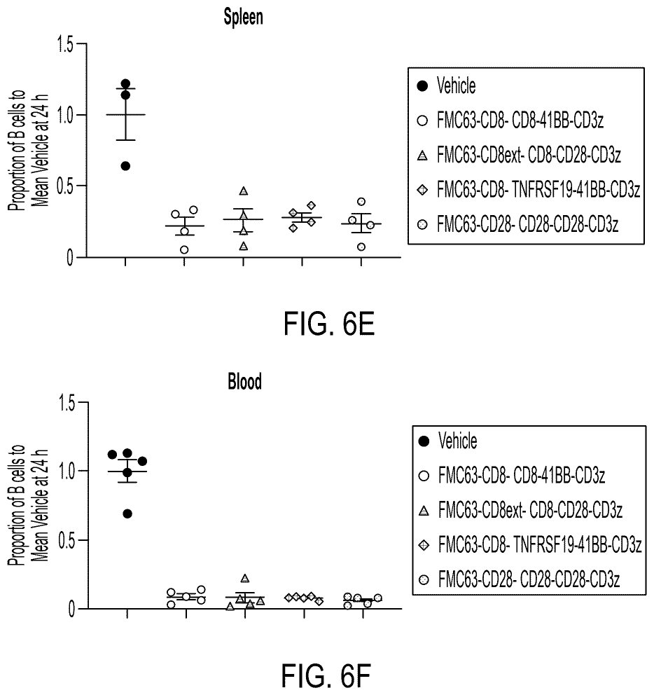

FIG. 6A is a schematic of CAR architectures such as exemplary anti-CD19 CAR architectures comprising an anti-CD19 antigen-binding domain such as FMC63 scFv), and various exemplary transmembrane domains (e.g., CD8a, CD28, and TNFRSF19), costimulatory domains (e.g., 4-1BB and CD28), and signaling domains (e.g., CD3zeta, CD3delta, CD3epsilon, CD3gamma); and exemplary signaling peptides (e.g., CD8a and CSF2RA). Exemplary codon-optimized coding sequences using some of these CAR architectures are also provided in FIG. 6A.

For FIGS. 6B-6F, the following circRNAs encoding various anti-CD19 CAR architectures were tested: FMC63-CD8a spacer-CD8 transmembrane domain-41BB costimulatory domain-CD3z signaling domain (with CD8a signal peptide) (within SEQ ID NO: 175); FMC63-CD8a extended spacer-CD8a transmembrane domain-CD28 costimulatory domain-CD3z signaling domain (within SEQ ID NO: 230); FMC63-CD8 spacer-TNFRS19 transmembrane domain-41BB costimulatory domain-CD3zeta signaling domain (within SEQ ID NO: 232); and FMC63-CD28 spacer-CD28 transmembrane domain-CD28 costimulatory domain-CD3zeta signaling domain (within SEQ ID NO: 233). All of the circRNAs tested were conjugated to LNP composition LNP-2 (“Reference formulation” as described in Table 16), and conjugated to anti-CD5 targeting moiety VHH319.

FIG. 6B is a graph of anti-CD19 CAR expression in CD3⁺ T cells from spleens of CD34⁺ HSC-engrafted NSG humanized mice at 24 hours after administration of LNP-2 encapsulating 0.5 mg/kg circRNA encoding various exemplary CAR architectures described in Table 9.

FIG. 6C is a graph of anti-CD19 CAR expression in circulating CD3⁺ T cells of CD34⁺ HSC-engrafted NSG humanized mice at 24 hours after administration of 0.5 mg/kg circRNA encoding various exemplary CAR architectures.

FIG. 6D is a graph of number of anti-CD19 CAR positive T cells per 5×10⁵ splenocytes from spleens of CD34⁺ HSC-engrafted NSG humanized mice at 24 hours after administration of 0.5 mg/kg circRNA encoding various exemplary CAR architectures.

FIG. 6E is a graph of the proportion of B cells to mean B cell count of vehicle control present in spleens of CD34⁺ HSC-engrafted NSG humanized mice at 24 hours after administration of 0.5 mg/kg circRNA encoding various exemplary CAR architectures.

FIG. 6F is a graph of proportion of B cells to mean B cell count of vehicle control in peripheral blood of CD34⁺ HSC-engrafted NSG humanized mice at 24 hours after administration of 0.5 mg/kg circRNA encoding various exemplary CAR architectures.

The results in FIG. 6B and FIG. 6C showed CAR expression in blood and spleen with the exemplary CAR architectures tested. The results in FIG. 6D showed anti-CD19 CAR positive T cells, and FIG. 6E and FIG. 6F showed corresponding B cell depletion. These results showed that the RNA-LNPs comprising circRNA encoding various CAR architecture designs tested showed CAR expression and depleted B cells.

Overall, this example showed that circRNAs comprising various exemplary CAR architectures encapsulated in targeted LNPs achieved a range of CAR expression and B cell depletion in vivo in humanized mice.

### Example 7: Effect of PEG Composition and Phospholipid Amounts in LNP Formulation on Encoded Protein Expression In Vivo

A. Higher Total Mole Percentage (Mol %) of Long Chain Fatty Acid Lipid Anchor in Total PEG Increased On-Target Delivery to T Cell and Reduced Off-Target Delivery

This example illustrates with respect to LNP composition that a higher mol % of long-chain fatty acid as the lipid anchor component of the total PEG-lipid increased delivery to the T cells in the spleen and bone marrow (on-target organs), and reduced off-target delivery to the liver in vivo.

Briefly, 0.5 mg/kg of circRNA encoding a reporter protein (mGreenLantern) encapsulated in LNPs comprising different amounts of an exemplary long chain fatty acid lipid anchor component of the total PEG-lipid, e.g., 0.1% C18 PEG, 0.5% C18 PEG, and 1.0% C18 PEG, were administered to wild-type (WT) mice. Specifically, the LNP compositions tested were LNP-19 (with “0.1% C18 PEG” referring to 0.1% DSPE-PEG2000-maleimide), LNP-20 (with “0.5% C18 PEG” referring to 0.1% DSPE-PEG2000-maleimide plus 0.4% DSG-PEG2000), or LNP-21 (with “1.0% C18 PEG” referring to 0.1% DSPE-PEG2000-maleimide plus 0.9% DSG-PEG2000), as described in Table 16. The LNP composition also included ionizable lipid Compound 7, DSPC, Cholesterol, and the appropriate amount C14 PEG to reach 2.0% total PEG. The LNPs were conjugated to a reference full-length anti-CD5 antibody (53-7.3 antibody) and a “non-targeted” control was included, which did not have a targeting moiety conjugated to the LNP and contained all C14 PEG. The reporter protein expression levels were measured in the spleen, bone marrow, and liver of the mice 24 hours after administration of the circRNA.

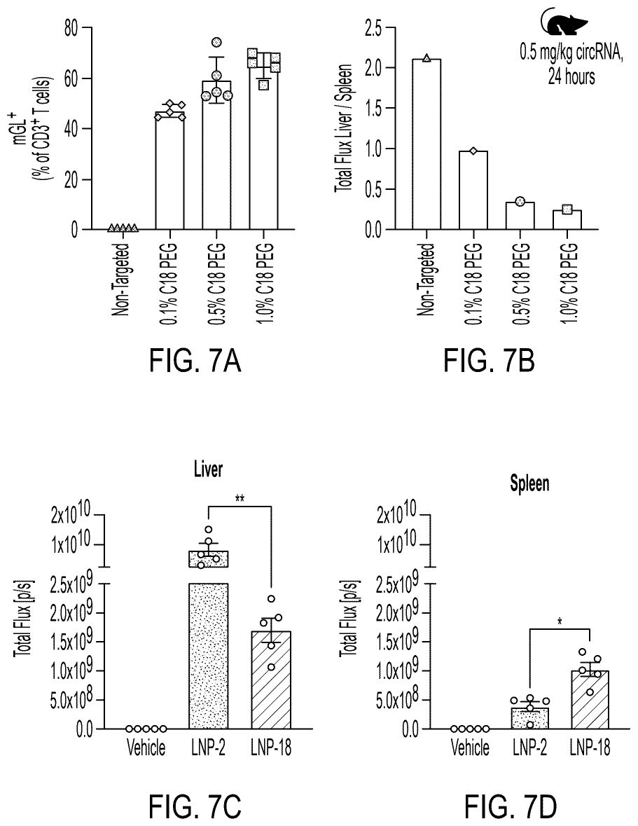

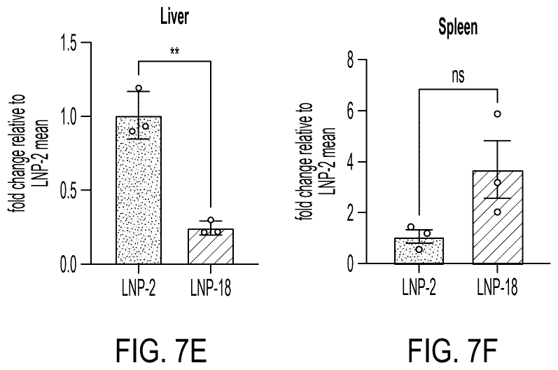

FIG. 7A shows percent mGreenLantern (mGL) positive cells as a percentage of total number of CD3+ T cells. As shown in FIG. 7A, as the C18 PEG percentage increased, the percentage of mGL+ cells expressing mGL (as a percentage of CD3+ T cells) increased. About 40% mGL+ expression was seen in the LNP comprising 0.1% C18 PEGs (LNP-19), about 55% mGL+ expression in the LNP comprising 0.5% C18 (LNP-20), and about 65% mGL+ expression in the LNP comprising 1.0% C18 PEG LNPs (LNP-21).

FIG. 7B shows total gene expression of the reporter protein in liver (off-target organ) relative to spleen (on-target organ). The non-targeted control showed high gene expression in the liver, while gene expression decreased with increasing percentage of C18 PEG in the LNP. The lowest flux in the liver compared to spleen was seen with 1% C18 PEG (LNP-21) relative to non-targeted control.

The results in FIG. 7A-7B collectively showed that an LNP with a higher mol % of C18 PEG resulted in increased delivery to T cells in the spleen and bone marrow, and reduced delivery to the liver of mice.

Overall, the results from this study showed that higher amounts of C18 PEG as the lipid anchor component of the LNP increased delivery to the T cell in vivo, e.g., in spleen and bone marrow, and reduced off-target delivery to the liver.

B. LNP Formulation with a Higher DSPC Mol % Reduced Off-Target Delivery and Enhanced In Vivo Biodistribution to T Cells (in the Spleen)

In this study, 0.5 mg/kg circRNA encoding a reporter protein (luciferase) was encapsulated in exemplary non-targeted LNPs, LNP-1, LNP-18 and LNP-22, as described in Table 16. First, the LNP properties of LNP-1, LNP-18 and LNP-22 were tested, as shown in Table 17A. The LNPs (with no targeting moieties or conjugation handles) were formulated and evaluated for properties such as size, polydispersity index (PDI), and percent encapsulation in vitro. Generally, satisfactory LNP properties include LNP size of less than or equal to about 101 nm, polydispersity index of less than or equal to 0.25, and percent encapsulation of greater than or equal to 95%. As shown in Table 17A, LNP-1, LNP-18 and LNP-22 showed satisfactory LNP properties.

TABLE 17A
LNP Composition and Properties relating to FIG. 7C and FIG. 7D

| LNP Composition | | | | | | | | |
|---|---|---|---|---|---|---|---|---|
| | % | | | | % DSPE- | LNP Properties | | |
| | ionizable | % | % | % DMG- | PEG2000- | Size | | % |
| LNP | lipid | DSPC | cholesterol | PEG2000 | Maleimide | (nm) | PDI | encapsulation |
| LNP-1 | 47.5% | 10% | 40.50% | 2% | 0% | 65.2 | 0.049 | 98.9 |
| LNP-18 | 39% | 20% | 39% | 1.9% | 0.1% | 68.9 | 0.044 | 98.8 |
| LNP-22 | 39% | 20% | 39% | 2% | 0% | 68.7 | 0.036 | 98.9 |

Then, circRNA-LNPs were administered intravenously to mice. The mice were also injected with 200 μL of luciferin at a concentration of 15 mg/mL for 10 to 15 minutes. The liver and spleen of the mice were harvested at 24 hours after administration of the RNA-LNP. After the organs were harvested, they were imaged using the IVIS imaging system, and luciferase expression was calculated as total flux (photon per second, p/s).

FIG. 7C is a graph showing total luciferase flux in the liver of the mice, and FIG. 7D is a graph showing total luciferase flux in the spleen of the mice.

The results in FIG. 7C showed that both LNP-18 and LNP-22 displayed decreased flux in liver, and the results in FIG. 7D showed that both LNP-18 and LNP-22 displayed increased flux in spleen compared with reference LNP-1.

In addition to the IVIS imaging, 4 hours after administration of the circRNA-LNP, the liver and spleen of mice were harvested. RNA was extracted from the liver and spleen and used for probe-based RT-qPCR analysis to determine the levels of circRNA that had been delivered to these organs. The circRNA levels were assessed with qPCR and normalized to Gapdh (glyceraldehyde-3-phosphatase dehydrogenase) signal from the same sample.

FIG. 7E is a graph showing circRNA levels in the liver of the mice, normalized to the mean value for circRNA-LNP-1. FIG. 7F is a graph showing circRNA levels in the spleen of the mice, normalized to the mean value for circRNA-LNP-1.

The results in FIG. 7E showed that both circRNA-LNP-18 and circRNA-LNP-22 displayed decreased circRNA delivery to the liver compared to circRNA-LNP-1. The results in FIG. 7F showed that both circRNA-LNP-18 and circRNA-LNP-22 displayed increased circRNA delivery to the spleen of the mice compared to circRNA-LNP-1.

Overall, these results showed that an LNP formulation with a higher DSPC mol % (e.g., LNP-18, LNP-22) was beneficial for reducing biodistribution to the liver (an off-target organ) and enhancing biodistribution to the spleen (a target organ).

### Example 8: Effect of Ionizable Lipids in LNP Formulation on Targeted Delivery and CAR Expression; and Efficiency of Targeted LNP Versus Non-Targeted LNP for Delivery to T Cells

A. Biodistribution of mRNA Expression of Exemplary Reporter Protein (mGL) in Extrahepatic Tissues and Immune Cells in RNA-LNP Formulations Using Exemplary Ionizable Lipids, without Addition of a Targeting Moiety

This example illustrated that delivery to CD45.2+ immune cells and gene expression was achieved by RNA-LNP formulations comprising exemplary ionizable lipids Compound 2 or Compound 7 without the addition of a targeting moiety.

In this example, 0.25 mg/kg or 0.5 mg/kg linear mGreenLantern or firefly luciferase RNA encapsulated in LNP comprising Compound 2 or Compound 7 was administered intravenously to mice, and the bone marrow, lung, lymph nodes, peripheral blood, and spleen of the mice were harvested at 24 hours post RNA-LNP administration. Cells were gated as CD45.2+ and analyzed by flow cytometry.

FIG. 8A is a graph showing mGreenLantern expression in CD45.2+ leukocytes in various extrahepatic tissues, such as bone marrow, lungs, peripheral blood, and spleen. As shown in FIG. 8A, expression of mGreenLantern in CD45.2+ leukocytes was highest in bone marrow followed by lungs, peripheral blood, and spleen. Comparable expression patterns in different tissues were observed for RNA-LNP comprising both ionizable lipid Compound 2 or Compound 7.

Further, CD45.2+ cells were gated based on cell-specific markers. FIG. 8B and FIG. 8C show graphs that indicate the percentage of mGL+CD4 T cells, CD8 T cells, B cells, NK cells, NKT cells, neutrophils, monocytes, macrophages, dendritic cells, and other CD45.2+ immune cells.

FIG. 8B shows cellular distribution as percentage of mGL+ cells in tissues after administration of 0.5 mg/kg linear RNA, which was conjugated to LNP comprising ionizable lipid Compound 2. FIG. 8C shows cellular distribution as percentage of mGL+ cells in tissues after administration of 0.5 mg/kg linear RNA, which was conjugated to LNP comprising ionizable lipid Compound 7.

The results in FIG. 8B and FIG. 8C showed that RNA-LNPs comprising ionizable lipid Compound 2 or Compound 7 both showed comparable patterns of cellular distribution of mGL expression.

Overall, the results showed comparable biodistribution in tissues and immune cells using RNA-LNPs comprising exemplary ionizable lipids Compound 2 or Compound 7.

B. In Vivo B-Cell Depletion by RNA-LNPs Comprising Exemplary Ionizable Lipids Compound 2 or Compound 7 with Addition of Anti-CD5 VHH319 Targeting Moiety

This example showed that CAR expression and B cell depletion was achieved in a CD34+ humanized NSG mouse model after administration of RNA-LNPs (comprising ionizable lipid Compound 2 or Compound 7) conjugated to an anti-CD5 targeting moiety (VHH319). In a CD34+ humanized NSG model, CD34+ hematopoietic stem cells (HSCs) isolated from human bone marrow donors, umbilical cord blood, or human fetal liver were engrafted in the murine bone marrow, resulting in robust human hematopoiesis, including development of T and B lymphocytes, NK cells, myeloid cells, and erythroblasts. Since the development and maturation of T cells occurs in the murine thymus, Graft-versus-host disease (GvHD) was significantly reduced in this model.

RNA-LNPs encapsulated in LNP-1 at a dose of 0.5 mg/kg circRNA (SEQ ID NO: 125) were administered intravenously in a CD34+ NSG mouse model. Anti-CD19 CAR expression was measured in spleen 24 hours after administration. FIG. 8D is a graph that shows anti-CD19 CAR expression in spleen as a percentage of CD3+ T cells. With the inclusion of an anti-CD5 targeting moiety (VHH319), LNPs comprising ionizable lipid Compound 2 or Compound 7 both showed targeting to the spleen.

FIG. 8E is a graph that shows that corresponding B cell depletion in spleen after administration of RNA-LNPs comprising ionizable lipid Compound 2 was comparable to RNA-LNPs comprising ionizable lipid Compound 7.

FIG. 8F is a graph that shows anti-CD19 CAR positive cells as a percentage of total CD3+ T cells in multiple studies carried out with circRNAs (SEQ ID NO: 175) conjugated to LNP-1 “Base LNP”, which refers to a non-targeted LNP with the LNP composition described in Table 16, compared to a circRNA (SEQ ID NO: 175) conjugated to targeted LNP (anti-CD5 targeting moiety VHH319), both comprising ionizable lipid Compound 2. A PBS-treated vehicle control was also included. The results in FIG. 8F showed that after administration of circRNA-LNP (SEQ ID NO: 175) wherein the LNP comprises ionizable lipid Compound 2, the CAR expression increased to between about 30%-50% relative to vehicle control. The results showed that anti-CD19 CAR expression after administration of the exemplary circRNA-LNP conjugated to the anti-CD5 targeting moiety (VHH319) was consistently higher than the non-targeted Base LNP control.

FIG. 8G is a graph that shows anti-CD19 CAR expression (MFI) among total CD3+ T cells in multiple studies carried out with circRNA (SEQ ID NO: 175) conjugated to LNP-1 (Base LNP) or targeted LNP (anti-CD5 targeting moiety VHH319), both comprising ionizable lipid Compound 2.

FIG. 8H is a graph that shows that B cell depletion (as a proportion of mean vehicle B cells at 24 hours after administration) was achieved in multiple studies carried out with circRNAs (SEQ ID NO: 175) conjugated to LNP-1 (Base LNP) or targeted LNP (anti-CD5 targeting moiety VHH319), both comprising Compound 2.

In both FIG. 8G and FIG. 8H, the results showed that anti-CD19 CAR expression and B cell depletion after administration of the exemplary circRNA-LNP conjugated to the anti-CD5 targeting moiety (VHH319) was consistently higher than the non-targeted Base LNP control.

Overall, the results showed that administration of RNA-LNPs comprising exemplary ionizable lipids, Compound 2 or Compound 7, and conjugated to an anti-CD5 targeting moiety (VHH319), achieved CAR expression and B cell depletion.

C. In Vivo Targeting Efficiency of circRNA Formulated in CD5-Targeted LNP to CD4+ and CD8+ T Cells

This in vivo study evaluated the targeting efficiency of circRNA formulated in a CD5-targeted LNP into T cells. CircRNA encoding mGreenLantern protein was formulated into either a base LNP without a targeting moiety (Non-targeted LNP) or a targeted LNP comprising Compound 2 utilizing a full-length anti-mouse CD5 antibody conjugated to the LNP (T cell-targeted LNP).

Briefly, Balb/C mice were administered with 0.5 mg/kg dose of circRNA (encoding mGL) via intravenous administration. Then, 24 hours later, the animals were euthanized to assess mGL expression in both CD4+ and CD8+ T cells present in immune organs, including lymph node and spleen. Flow cytometric analysis was performed to quantify the level of mGL expression after circRNA delivery via non-targeted vs T cell-targeted LNPs.

FIG. 8I-FIG. 8K are flow cytometry data showing the increased targeting efficiency of circRNA formulated in a CD5-targeted LNP targeting to CD4+ and CD8+ T cells in the spleen of mice. The mice were administered via intravenous administration 0.5 mg/kg dose of circRNA (encoding mGL) formulated in an anti-CD5 targeted LNP or a vehicle control, and the level of mGL expression was measured. In the spleen, circRNA formulated with T cell targeted LNP (via an anti-CD5 targeting moiety) resulted in about a 14-fold to about a 24-fold increase in delivery to CD4+ and CD8+ T cells, as shown in FIG. 8I-FIG. 8K.

FIG. 8L-FIG. 8N are flow cytometry data showing the increased targeting efficiency of circRNA formulated in a CD5-targeted LNP targeting to CD4+ and CD8+ T cells in the lymph node of mice. The mice were administered via intravenous administration 0.5 mg/kg of circRNA (encoding mGL) or a vehicle control, and the level of mGL expression was measured. In the lymph nodes, circRNA formulated with T cell-targeted LNP (via an anti-CD5 targeting moiety) resulted in about a 23-fold to about a 30-fold increase in delivery to CD4+ and CD8+ T cells, as shown in FIG. 8L-FIG. 8N.

Overall, the results from this study showed about a 14-fold to about a 30-fold increase in targeted delivery of circRNA to CD4+ and CD8+ T cells in the spleen and lymph node of mice with a targeted LNP as compared to a non-targeted LNP.

### Example 9: Effect of LNP Composition on Potency and Biodistribution

### A. Effect of Phospholipid (Distearoylphosphatidylcholine (DSPC)) Amounts in the LNP on Potency and Biodistribution

This example demonstrates the effect of the amount of distearoylphosphatidylcholine (DSPC), a phospholipid, in the LNP composition on potency and biodistribution.

LNPs with varying compositions, including mol % of DSPC from 5.4%-33.8%, as described in Table 17B below were formulated and evaluated for properties such as size, polydispersity, and percent of encapsulation in vitro. Generally, satisfactory LNP properties included LNP size of less than or equal to about 101 nm, polydispersity index of less than or equal to 0.25, and percent encapsulation of greater than or equal to 95%. As shown in Table 17B, all of the LNP formulations showed satisfactory LNP properties.

TABLE 17B
LNP Composition and Properties (size, polydispersity index (PDI), percent (%) encapsulation)

| | LNP Composition | | | | | | | | |
|---|---|---|---|---|---|---|---|---|---|
| | % | | | | % DSPE- | | LNP Properties¹ | | |
| | ionizable | % | % | % DMG- | PEG2000- | % DSG- | Size | | % |
| LNP² | lipid | DSPC | cholesterol | PEG2000 | Maleimide | PEG2000 | (nm) | PDI | encapsulation |
| LNP-1 | 47.5% | 10% | 40.5% | 2% | 0% | 0% | 82.8 | 0.22 | 98.3 |
| (“Base | | | | | | | | | |
| LNP”) | | | | | | | | | |
| LNP-2 | 47.5% | 10% | 40.5% | 1.5% | 0.5% | 0% | 76.4 | 0.13 | 98.2 |
| (“Reference | | | | | | | | | |
| formulation”) | | | | | | | | | |
| LNP-3 | 60% | 8% | 30% | 1.5% | 0.5% | 0% | 93.6 | 0.05 | 96.8 |
| LNP-4 | 46% | 15% | 37% | 1.5% | 0.5% | 0% | 71.5 | 0.11 | 98.1 |
| LNP-5 | 45% | 5% | 48% | 1.5% | 0.5% | 0% | 87.2 | 0.16 | 96.3 |
| LNP-6 | 41% | 13% | 44% | 1.5% | 0.5% | 0% | 69 | 0.07 | 97.3 |
| LNP-7 | 31% | 18% | 49% | 1.5% | 0.5% | 0% | 88.6 | 0.15 | 98.4 |
| LNP-8 | 26% | 26% | 46% | 1.5% | 0.5% | 0% | 84.6 | 0.08 | 99 |
| LNP-9 | 33% | 23% | 42% | 1.5% | 0.5% | 0% | 76.1 | 0.07 | 98.7 |
| LNP-10 | 25% | 33% | 40% | 1.5% | 0.5% | 0% | 90.8 | 0.07 | 98.8 |
| LNP-11 | 39% | 20% | 39% | 1.5% | 0.5% | 0% | 69.2 | 0.03 | 98.2 |
| LNP-12 | 47% | 25% | 26% | 1.5% | 0.5% | 0% | 85.5 | 0.05 | 98.2 |
| LNP-13 | 40.5% | 29.6% | 27.9% | 1.5% | 0.5% | 0% | 87.9 | 0.05 | 98.3 |
| LNP-14 | 33% | 34% | 31% | 1.5% | 0.5% | 0% | 93.2 | 0.05 | 97.3 |
| LNP-15 | 43% | 23% | 32% | 1.5% | 0.5% | 0% | 79.3 | 0.02 | 98.7 |
| LNP-16 | 28% | 39% | 31% | 1.5% | 0.5% | 0% | 100.1 | 0.07 | 95 |
| LNP-17 | 35% | 28% | 35% | 1.5% | 0.5% | 0% | 83.2 | 0.03 | 98.8 |
| LNP-18 | 39% | 20% | 39% | 1.9% | 0.1% | 0% | 70.5 | 0.03 | 99.1 |
| LNP-19 | 47.5% | 10% | 40.5% | 1.9% | 0.1% | 0% | 81.9 | 0.028 | 98.5 |
| LNP-20 | 47.5% | 10% | 40.5% | 1.5% | 0.1% | 0.4% | 84.3 | 0.038 | 98.8 |
| LNP-21 | 47.5% | 10% | 40.5% | 1.0% | 0.1% | 0.9% | 88.1 | 0.027 | 98.4 |

¹Unless otherwise noted, the LNP properties listed for the LNP compositions refer to the LNP comprising Compound 7 as the ionizable lipid.
²LNP-1 is a Base LNP with no targeting moiety. For LNP-2 to LNP-18, the LNP compositions were conjugated to anti-CD5 targeting moiety VHH319 via a GGGSCAAA conjugation handle. For LNP-19 to LNP-21, the LNP compositions were conjugated to a reference full-length anti-CD5 monoclonal antibody (53-7.3 antibody), and no conjugation handle was used.

The LNPs were down-selected based on particle stability and in vitro potency in Jurkat T cells, and exemplary LNPs were administered intravenously to mice to test for potency and biodistribution.

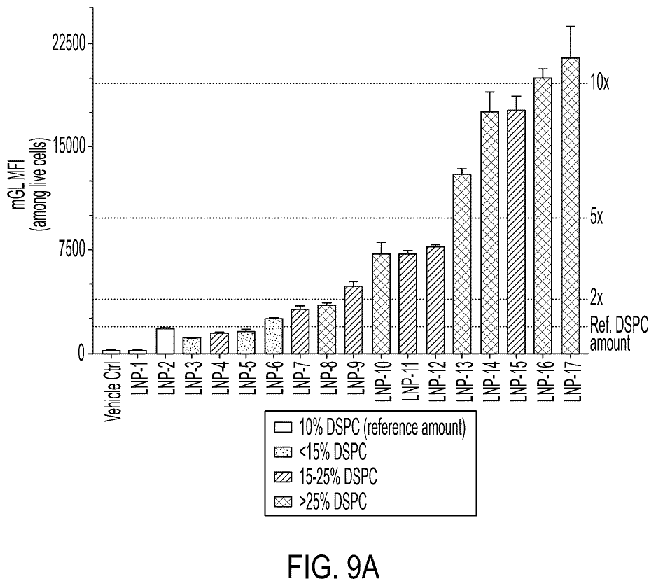

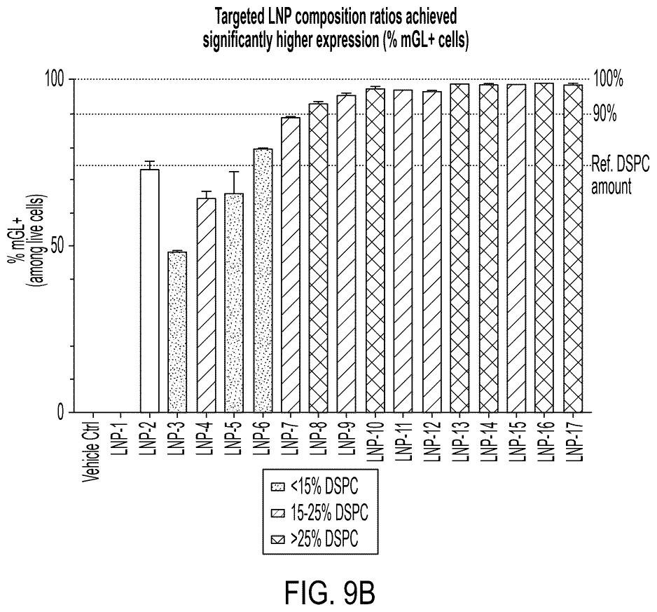

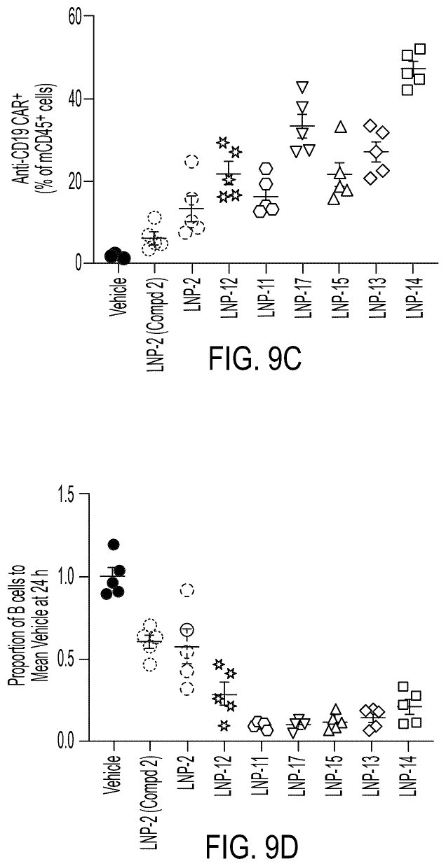

FIG. 9A is a graph of mGreenLantern expression (gMFI) in Jurkat T cells by circRNAs encapsulated in various exemplary LNP formulations described in Table 16 and shows that increasing the DSPC from the reference amount of about 10% DSPC to about 15% to about 25% increased mGL expression, and increasing to greater than 25% DSPC further increased the mGL expression in Jurkat T cells.

FIG. 9B is a graph of % mGL positive (among live cells) in Jurkat T cells by circRNAs encapsulated in various exemplary LNP formulations described in Table 16. In this in vitro study, 1 ng circRNA (encoding mGL reporter) per 1×10⁵ Jurkat cells was administered. LNP formulations including the reference 10% DSPC showed about 75% mGL positive cells. At 15-25% DSPC, most LNP formulations tested showed increases over the reference 10% DSPC. At greater than 25% DSPC, all LNP formulations tested showed greater than 90% mGL positive cells. The results in FIG. 9A and FIG. 9B showed that increasing DSPC mol % from the reference 10% DSPC in LNP formulations increased potency.

Further, CD34+ mice were administered 0.5 mg per kg body weight of exemplary RNA-LNP formulations, and anti-CD19 CAR expression and B cell depletion in spleen was measured 24 hours after administration. First, before administration to mice of the RNA-LNP formulations, the LNP properties of the LNP compositions were tested, as shown in Table 17C. All the LNPs in Table 17C included an anti-CD5 targeting moiety VHH319 with a C-terminal G3S-Cys-AAA (GGGSCAAA, SEQ ID NO: 122) as the conjugation handle. The LNPs were formulated and evaluated for properties such as size, PDI, and percent of encapsulation. Generally, satisfactory LNP properties include LNP size of less than or equal to about 101 nm, PDI of less than or equal to 0.25, and percent encapsulation of greater than or equal to 95%. As shown in Table 17C, all of the LNP compositions showed satisfactory LNP properties.

TABLE 17C
LNP Composition and Properties relating to FIG. 9C

| LNP Composition¹ | | | | | | | | |
|---|---|---|---|---|---|---|---|---|
| | | | | | % DSPE- | LNP Properties | | |
| | % ionizable | % | % | % DMG- | PEG2000- | Size | | % |
| LNP | lipid | DSPC | cholesterol | PEG2000 | Maleimide | (nm) | PDI | encapsulation |
| LNP-2 | 47.5 | 10% | 40.5% | 1.5% | 0.5% | 62.1 | 0.05 | 97.3 |
| (Compound 2) | (Compound 2) | | | | | | | |
| LNP-2 | 47.5 | 10% | 40.5% | 1.5% | 0.5% | 65.6 | 0.04 | 98.1 |
| | (Compound 7) | | | | | | | |
| LNP-12 | 47 | 25% | 26% | 1.5% | 0.5% | 87.8 | 0.06 | 98.1 |
| | (Compound 7) | | | | | | | |
| LNP-11 | 39 | 20% | 39% | 1.5% | 0.5% | 68.9 | 0.05 | 98.3 |
| | (Compound 7) | | | | | | | |
| LNP-17 | 35 | 28% | 35% | 1.5% | 0.5% | 81.4 | 0.03 | 98.9 |
| | (Compound 7) | | | | | | | |
| LNP-15 | 43 | 23% | 32% | 1.5% | 0.5% | 77.5 | 0.05 | 98.6 |
| | (Compound 7) | | | | | | | |
| LNP-13 | 40.5 | 29.6% | 27.9% | 1.5% | 0.5% | 92.2 | 0.04 | 98.7 |
| | (Compound 7) | | | | | | | |
| LNP-14 | 33 | 34% | 31% | 1.5% | 0.5% | 94.2 | 0.05 | 98.1 |
| | (Compound 7) | | | | | | | |

¹All LNP compositions tested were conjugated to anti-CD5 targeting moiety VHH319 via a GGGSCAAA conjugation handle.

Then, the RNA-LNP formulations were administered to the mice, and the results obtained are depicted in FIG. 9C. FIG. 9C is a graph of anti-CD19 CAR positive cells as a percentage of mCD45+ cells showing anti-CD19 CAR expression with various LNP formulations relative to a vehicle control. For both FIG. 9C and FIG. 9D), the majority of the LNP formulations tested (LNP-2, LNP-11, LNP-12, LNP-13, LNP-14, LNP-15, and LNP-17) comprise ionizable lipid Compound 7; an LNP-2 formulation comprising ionizable lipid Compound 2 (“LNP-2 (Compd 2)”) was also tested.

FIG. 9D is a graph of proportion of B cells (relative to mean vehicle) remaining in vivo in spleen after administration of exemplary circRNA (encoding for anti-CD19 CAR) conjugated to LNP comprising exemplary LNP formulations comprising between 20%-34% DSPC relative to reference LNP formulations comprising 10% DSPC and a vehicle control.

The results in FIG. 9C showed that LNP formulations 12-14 (as described in Table 17C) showed increased anti-CD19 CAR expression relative to vehicle control as well as reference LNP formulations (“LNP-2” means LNP-2 comprising Compound 7, and “LNP-2 (Compd 2)” means LNP-2 comprising Compound 2). The results in FIG. 9D showed that LNP formulations 12-14 from Table 16 comprising between 20%-34% DSPC showed increased B cell depletion relative to vehicle control as well as reference LNP formulations (“LNP-2” means LNP-2 comprising Compound 7, and “LNP-2 (Compd 2)” means LNP-2 comprising Compound 2)) comprising 10% DSPC.

Overall, this example showed that various LNP formulations, including an LNP formulation with a higher DSPC mol %, was beneficial for improving potency in vitro and in vivo. In other words, in vitro and in vivo potency (for, e.g., anti-CD19 expression and B cell depletion) was improved by using the LNP compositions as described in this example.

### Example 10. B Cell Depletion by Using RNA-LNPs Conjugated to Exemplary Targeting Moieties: Anti-CD2, Anti-CD5, Anti-CD7, and Anti-CD8

### A. RNA-LNPs Conjugated to Exemplary Anti-CD2, Anti-CD5, Anti-CD7 and Anti-CD8 Targeting Moieties Achieved B Cell Depletion.

This example demonstrates that exemplary anti-CD5 targeting moieties (e.g., anti-CD5 VHH (e.g., VHH319), and anti-CD5 full-length antibody), anti-CD2 full-length antibodies, anti-CD7 full-length antibodies, and anti-CD8 full-length antibodies depleted B cells.

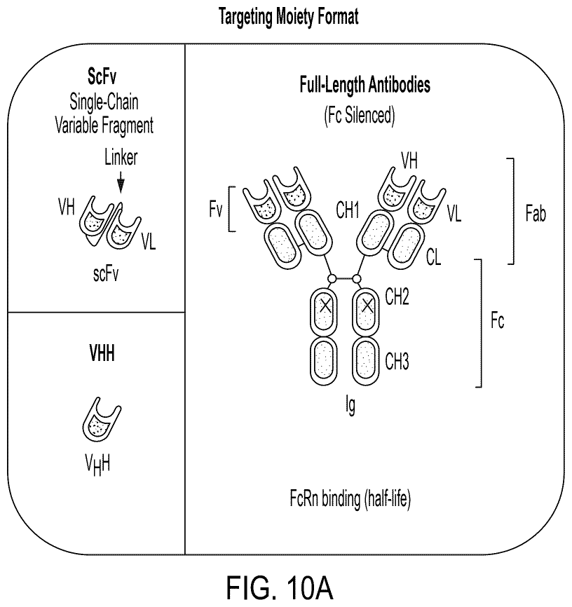

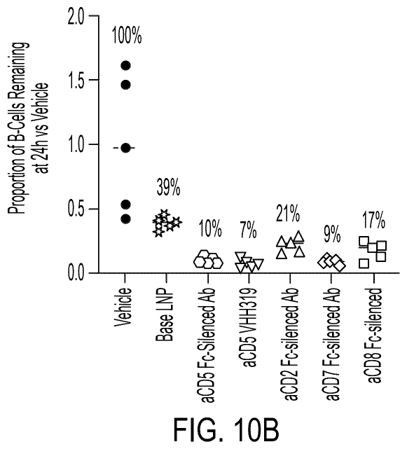

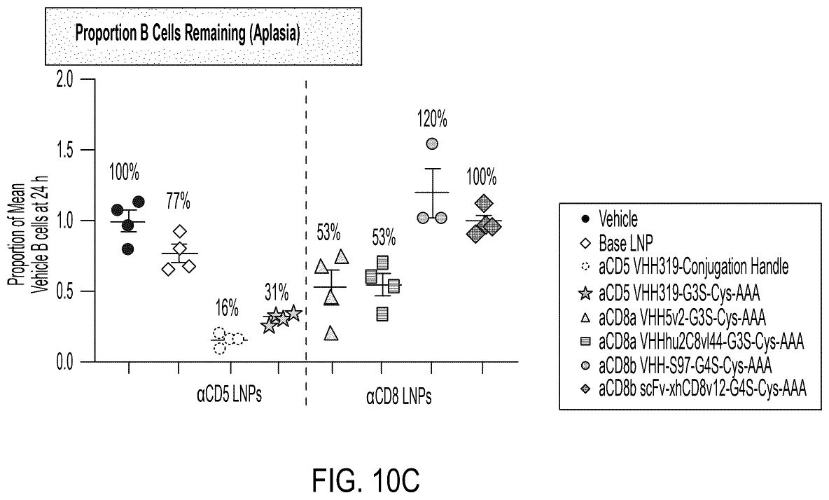

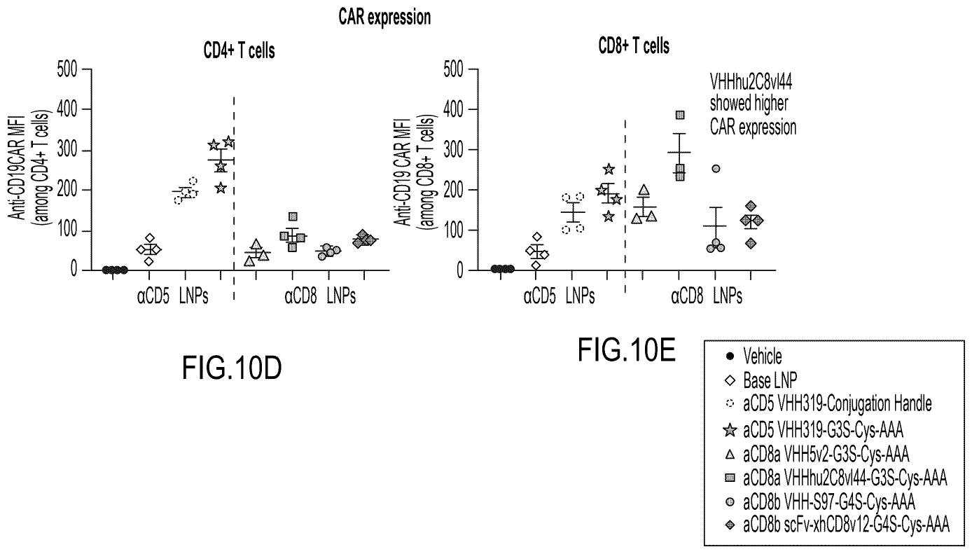

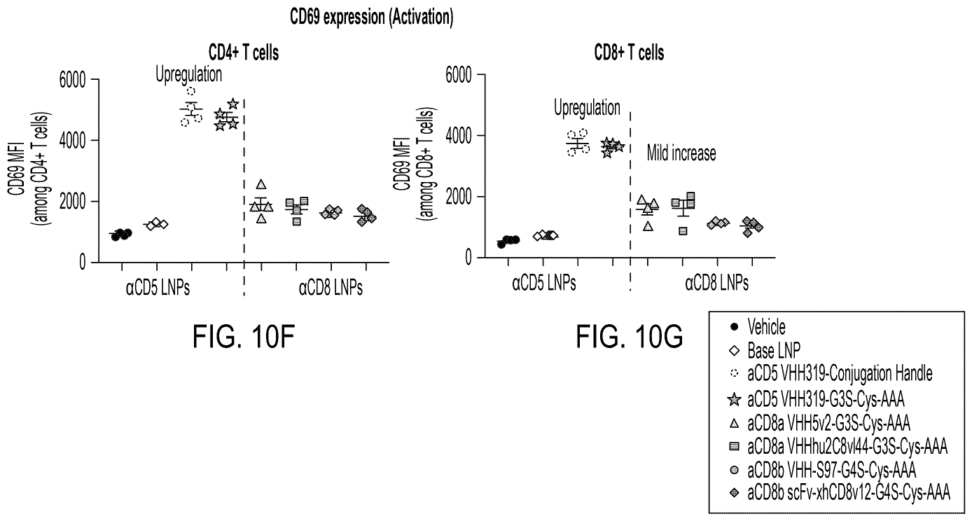

FIG. 10A is a schematic that shows the targeting moiety formats of exemplary targeting moieties, e.g., scFv, VHH, and full-length antibodies.

In this example, exemplary T cell targeting moieties, such as anti-hCD2 Fc silenced full-length antibody (35.1), anti-hCD5 Fc silenced full-length antibody (5D7), anti-hCD5 VHH319, anti-hCD7 Fc silenced full-length antibody (3A1E), and anti-hCD8 Fc silenced full-length antibody (OKT8) were conjugated to an LNP comprising ionizable lipid Compound 2. The circRNA construct encodes for an anti-human CD19 CAR and comprises the IRES of SEQ ID NO: 1.

The RNA-LNP was administered intravenously to CD34+ NSG mice at 1 mg/kg circRNA in a single dose. The effect of the antibody format/Fc silencing on lymphocyte depletion was evaluated 24 hours after administration.

FIG. 10B is a graph of percent B cells remaining (as a percentage of vehicle) 24 hours after administration of RNA-LNPs comprising various exemplary targeting moieties (anti-CD2, anti-CD5, anti-CD7 and anti-CD8). The anti-CD5 targeting moiety VHH319 performed as well as full-length anti-CD5, anti-CD2, anti-CD7 and anti-CD8 antibodies. The results in FIG. 10B showed that B cell depletion was observed regardless of Fc silencing or choice of T cell receptor. Targeting with anti-CD5 targeting moiety VHH319 resulted in 7% B cells remaining, meaning 93% B cell depletion was achieved. Targeting with anti-CD5 Fc silenced antibody showed 10% B cells remaining (90% B cell depletion). Targeting with anti-CD2 Fc silenced antibody showed 21% B cells remaining (79% B cell depletion). Targeting with anti-CD7 silenced antibody showed 9% B cells remaining (91% B cell depletion). Targeting with anti-CD8 silenced antibody showed 17% B cells remaining (83% B cell depletion).

B. Evaluation of Anti-CD5 and Anti-CD8 Antibody Fragments with “G3S-Cys-AAA” and “G4S-Cys-AAA” Conjugation Handles

This study evaluated an exemplary anti-CD5 VHH and exemplary anti-CD8 scFv and full length antibodies with similar conjugation handles for CAR expression, T cell activation, and B cell depletion.

In this study, CD34+ NSG mice were administered intravenously 0.5 mg/kg of circRNA (SEQ ID NO: 175) formulated in LNPs comprising ionizable lipid Compound 2 conjugated with anti-CD5 or anti-CD8 targeting moieties, and exemplary anti-CD5 and anti-CD8 targeting moieties with similar conjugation handles were compared. Table 11 provides the sequences for these exemplary targeting moieties.

FIG. 10C is a graph showing proportion of B cells remaining in spleen 24 hours after administration of RNA-LNP conjugated to two exemplary targeting moieties, anti-CD5 and anti-CD8, with a “G3S-Cys-AAA” (SEQ ID NO: 120) or “G4S-Cys-AAA” (SEQ ID NO: 343) conjugation handle (as described in Table 12). Anti-CD5 VHH319 with a “G3S-Cys-AAA” (SEQ ID NO: 120) conjugation handle showed 31% B cells remaining, and two of the anti-CD8 VHHs both showed 53% B cells remaining.

FIG. 10D is a graph showing anti-CD19 CAR MFI among CD4+ T cells. The results in FIG. 10D showed high CAR expression in CD4+ T cells by administering circRNA encapsulated by LNP conjugated to exemplary anti-CD5 VHH319 comprising a “G3S-Cys-AAA” conjugation handle and a reference conjugation handle (containing a HHHHHH tag).

FIG. 10E is a graph showing anti-CD19 CAR (MFI) among CD8+ T cells. The results in FIG. 10E showed high CAR expression in CD8+ T cells by administering circRNA encapsulated by LNP conjugated to exemplary anti-CD5 VHH319 comprising a “G3S-Cys-AAA” conjugation handle and a reference conjugation handle.

FIG. 10F is a graph showing CD69 MFI (among CD4+ T cells) as a marker of T cell activation. The results in FIG. 10F showed high activation of CD4+ T cells by administering circRNA encapsulated by LNP conjugated to exemplary anti-CD5 VHH319 comprising a G3S-Cys-AAA conjugation handle and a reference conjugation handle.

FIG. 10G is a graph showing CD69 MFI (among CD8+ T cells) as a marker of T cell activation. The results in FIG. 10G showed high activation of CD8+ T cells by administering circRNA encapsulated by LNP conjugated to exemplary anti-CD5 VHH319 comprising a G3S-Cys-AAA″ conjugation handle and a reference conjugation handle.

Overall, this example demonstrated efficacy of RNA-LNPs conjugated to various exemplary T cell targeting moieties in achieving CAR expression, T cell activation, and B cell depletion.

### Example 11: B-Cell Depletion by Use of RNA-LNPs Comprising Exemplary Modified Anti-CD5 VHH T Cell Targeting Moieties

This example demonstrated B-cell depletion by RNA-LNPs conjugated to exemplary modified anti-CD5 VHH T cell targeting moieties.

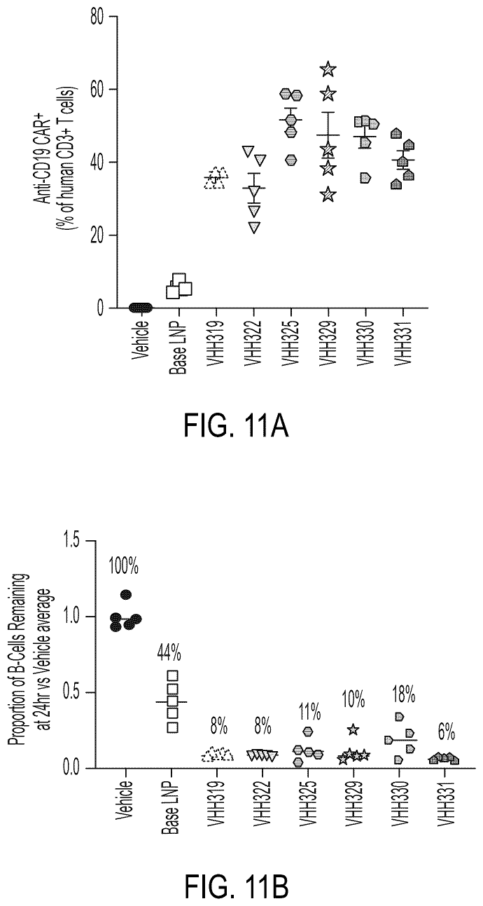

FIG. 11A is a graph showing the percentage of anti-CD19 CAR+ cells relative to total human CD3+ T cells 24 hours after RNA-LNP (SEQ ID NO: 175) administration; the LNPs comprised ionizable lipid Compound 2 and was conjugated to various exemplary anti-CD5 modified VHH antibodies as targeting moieties: VHH319, VHH322, VHH325, VHH329, VHH330, and VHH331 described in Table 10. The vehicle is PBS buffer, and “Base LNP” is the LNP without a targeting moiety.

FIG. 11B is a graph showing the proportion of B cells remaining after 24 hours of RNA-LNP administration relative to vehicle control for RNA-LNPs conjugated to various exemplary anti-CD5 modified VHH antibodies as targeting moieties: VHH319, VHH322, VHH325, VHH329, VHH330 and VHH331 described in Table 10. The vehicle is PBS buffer, and “Base LNP” is the LNP without a targeting moiety.

Overall, this example demonstrated that RNA-LNPs conjugated to several modified anti-CD5 VHHs of the present disclosure were highly effective in targeting to T cells and depleting B cells.

### Example 12: Using Various Conjugation Handles (to Conjugate the Targeting Moiety to the LNP) Resulted in Comparable In Vivo CAR Expression and B Cell Depletion

This example demonstrated the effect of various exemplary conjugation handles for conjugating the targeting moiety (e.g., VHH319) to the LNP on CAR expression and B cell depletion.

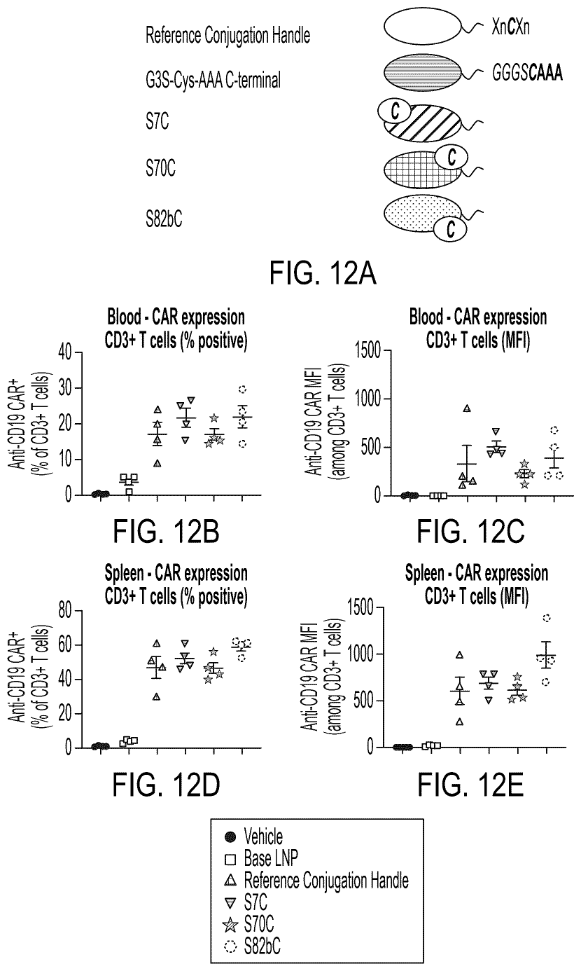

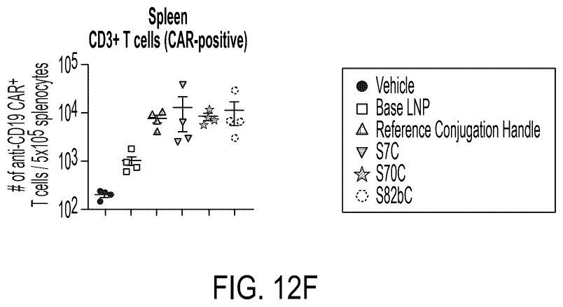

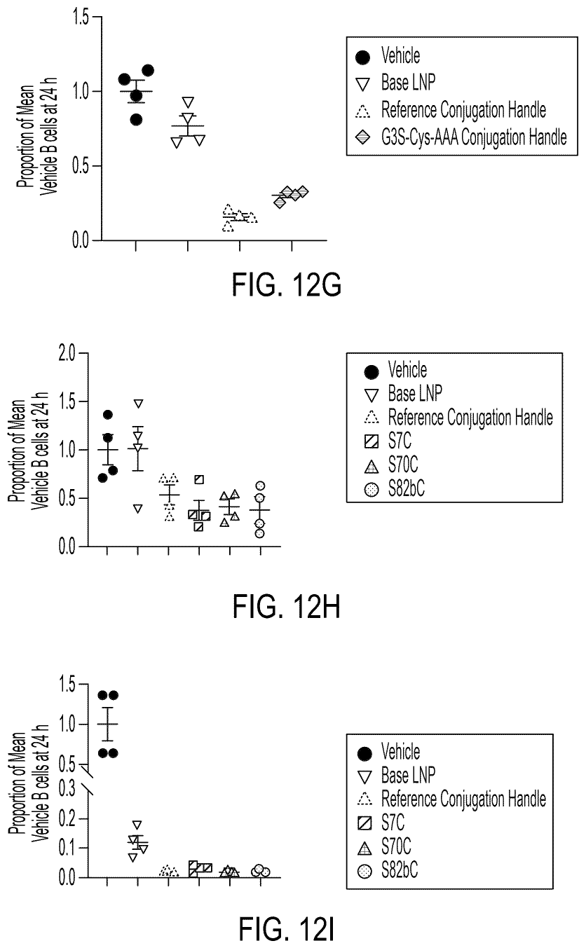

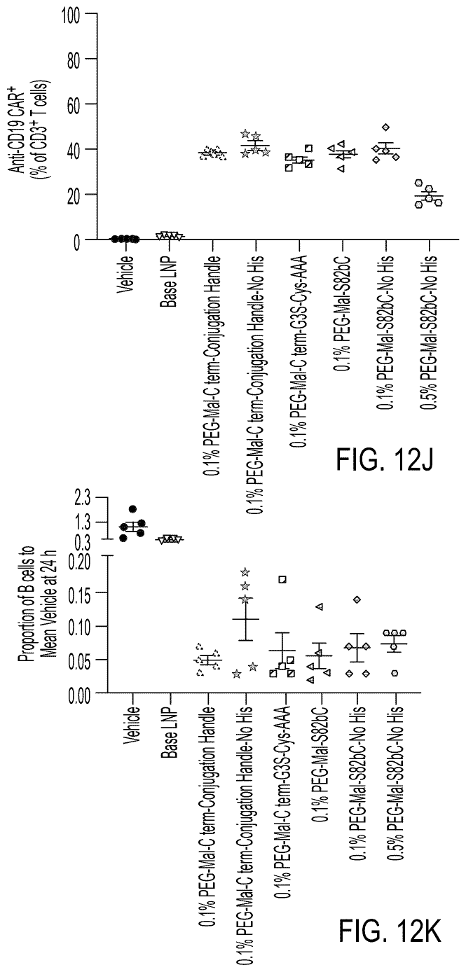

FIG. 12A is a schematic of various engineered cysteine locations used to conjugate the targeting moiety, e.g., VHH, (shown as ovals) to the delivery vehicle, e.g, LNP, via conjugation handles, as shown in Table 12. For example, the cysteine locations included: being adjacent to the conjugation handle, which was at the C-terminus of the VHH (e.g, as in “G3S-Cys-AAA” (SEQ ID NO: 120) conjugation handle) or internal to the VHH, e.g., S7C, S70C, and S82bC. For the reference conjugation handle, the cysteine location was at the C-terminus of the VHH.

CD34+ NSG mice were intravenously administered circRNA (SEQ ID NO: 175) encapsulated by LNP with the exemplary VHH conjugation handles described herein at a dose of 0.5 mg/kg of the circRNA. The spleen and blood of mice were evaluated 24 hours after administration for CAR expression and B cell depletion.

FIG. 12B is a graph showing percentage of anti-CD19 CAR+ cells relative to total CD3+ T cells in blood.

FIG. 12C is a graph showing percentage of anti-CD19 CAR (MFI) (among CD3+ T cells) in blood.

FIG. 12D is a graph showing percentage of anti-CD19 CAR+ cells relative to total CD3+ T cells in spleen.

FIG. 12E is a graph showing percentage of anti-CD19 CAR (MFI) among CD3+ T cells in spleen.

FIG. 12F is a graph showing number of anti-CD19 CAR+ T cells per 5×10⁵ splenocytes in spleen.

FIG. 12G is a graph showing proportion of B cells remaining in spleen relative to vehicle control when administered RNA-LNP conjugated to exemplary modified anti-CD5 targeting moiety VHH319 with different conjugation handles—a VHH319 with a reference conjugation handle, and a VHH319 with a “G3S-Cys-AAA” (SEQ ID NO: 120) conjugation handle were compared. The results in FIG. 12G showed that the anti-CD5 targeting moiety VHH319 with a “G3S-Cys-AAA” (SEQ ID NO: 120) handle was comparable to a VHH319 with a reference conjugation handle in depleting B cells.

FIG. 12H is a graph showing proportion of B cells remaining in spleen relative to vehicle control when administered RNA-LNP conjugated to exemplary modified anti-CD5 targeting moiety VHH319-VHH319 with S7C, S70C, and S82bC, and a control VHH319 with a reference conjugation handle.

FIG. 12I is a graph showing proportion of B cells remaining in blood relative to vehicle control when administered RNA-LNP comprising exemplary modified anti-CD5 VHH319 targeting moieties—VHH319 comprising S7C, S70C and S82bC and a control VHH319 comprising a reference conjugation handle.

FIG. 12J is a graph showing anti-CD19 CAR expression in spleen as a percentage of CD3+ T cells when administered RNA-LNP conjugated to the targeting moiety using various exemplary conjugation handles for conjugating the targeting moiety, namely, 0.1% PEG maleimide C-terminal conjugation, with and without a histidine tag, 0.1% PEG-maleimide C-terminal “G3S-Cys-AAA” (SEQ ID NO: 120) conjugation handle, 0.1% PEG maleimide S82bC conjugation with and without histidine tag, and a 0.5% PEG maleimide S82bC conjugation without a histidine tag, compared to a vehicle control and Base LNP control. Before the RNA-LNP was administered to mice, first, the LNP properties of the LNP compositions had been tested, as shown in Table 17D. As shown in Table 17D, all of the LNP compositions had shown satisfactory LNP properties.

TABLE 17D
LNP compositions and properties relating to FIG. 12J

| | | | | | | VHH319 | | | |
|---|---|---|---|---|---|---|---|---|---|
| | | | | | | Targeting | | | |
| LNP Composition | | | | | | Moiety | | | |
| | % | | | | % DSPE- | conjugated to | LNP Properties | | |
| | ionizable | % | % | % DMG- | PEG2000- | conjugation | Size | | % |
| LNP | lipid | DSPC | cholesterol | PEG2000 | Maleimide | handle | (nm) | PDI | encapsulation |
| LNP-22 | 39 | 20 | 39 | 2 | 0 | Non- | 66.3 | 0.05 | 96.4 |
| | | | | | | targeted (no | | | |
| | | | | | | targeting | | | |
| | | | | | | moiety or | | | |
| | | | | | | conjugation | | | |
| | | | | | | handle) | | | |
| LNP-18 | 39 | 20 | 39 | 1.9 | 0.1 | Reference | 70.5 | 0.04 | 99.2 |
| | | | | | | conjugation | | | |
| | | | | | | handle (with | | | |
| | | | | | | His tag) | | | |
| LNP-18 | 39 | 20 | 39 | 1.9 | 0.1 | GGGGSGGGGSCAS | 70.4 | 0.03 | 98.9 |
| | | | | | | (SEQ ID | | | |
| | | | | | | NO: 345) | | | |
| | | | | | | conjugation | | | |
| | | | | | | handle (no | | | |
| | | | | | | Histidine | | | |
| | | | | | | tag) | | | |
| LNP-18 | 39 | 20 | 39 | 1.9 | 0.1 | GGGSCAAA | 70.5 | 0.03 | 99.1 |
| | | | | | | (SEQ ID | | | |
| | | | | | | NO: 120) | | | |
| LNP-18 | 39 | 20 | 39 | 1.9 | 0.1 | Internal C | 70.2 | 0.03 | 99.5 |
| LNP-18 | 39 | 20 | 39 | 1.9 | 0.1 | Internal C | 70.6 | 0.03 | 99.3 |
| | | | | | | (no | | | |
| | | | | | | Histidine | | | |
| | | | | | | tag) | | | |
| LNP-11 | 39 | 20 | 39 | 1.5 | 0.5 | Internal C | 73.1 | 0.07 | 99.3 |
| | | | | | | (no | | | |
| | | | | | | Histidine | | | |
| | | | | | | tag) | | | |

FIG. 12K is a graph showing proportion of B cells remaining in spleen relative to vehicle control when administered RNA-LNP conjugated to the targeting moiety via exemplary conjugation chemistries, namely, 0.1% PEG maleimide C-terminal conjugation, with and without a histidine tag, 0.1% PEG-maleimide C-terminal “G3S-Cys-AAA” (SEQ ID NO: 120) conjugation handle, 0.1% PEG maleimide S82bC conjugation with and without histidine tag, and a 0.5% PEG maleimide S82bC conjugation without histidine tag, compared to a vehicle control and Base LNP control.

Overall, the results from this example showed comparable CAR expression and B cell depletion in the blood and spleen of mice after administration of RNA-LNPs conjugated to the targeting moiety VHH319, where various engineered cysteine locations were used for conjugating the VHH to the LNP.

### Example 13: Exemplary Conjugation Chemistries for Conjugation of a Targeting Moiety and Delivery Vehicle

This example demonstrated the effect of exemplary conjugation chemistries, such as thiol/maleimide, azide-DBCO, and sortase-catalyzed conjugation chemistries, in conjugating the anti-CD5 targeting moiety (e.g., VHH319) to the LNP.

FIG. 13A shows a schematic of exemplary conjugation chemistries for conjugating a targeting moiety to a delivery vehicle, e.g., LNP. For example, for the “C-terminal cysteine” conjugation, the VHH is conjugated to the lipid nanoparticle via a conjugation handle of SEQ ID NO: 120 which comprises a thiol reactive group. The PEG in the lipid nanoparticle is “modified” (and thus “functionalized”; the terms “modified” and “functionalized” are used interchangeably herein) with maleimide, which is conjugated to an amino acid (e.g., cysteine) in the conjugation handle through the thiol reactive group via thiol-maleimide conjugation. For the “internal cysteine” conjugation, the VHH sequence is engineered to comprise an internal cysteine residue, which is then similarly conjugated via thiol reactive group to a maleimide-modified PEG via thiol-maleimide conjugation.

In FIG. 13A, for the “C-terminal tetrazine” conjugation, for example, in tetrazine ligation, a VHH bearing a tetrazine moiety undergoes an inverse electron-demand Diels-Alder (IEDDA) reaction with a trans-cyclooctene (TCO)-functionalized LNP. This highly selective, bioorthogonal reaction proceeds efficiently under physiological conditions, forming a stable covalent bond between the VHH and the LNP.

In FIG. 13A, for the “C-terminal azide” conjugation, the cysteine of a protein or peptide is chemically modified to attach an azide group (—N₃ group), enabling site-specific conjugation to other molecules through “click chemistry” reaction. The attached azide readily reacts with an alkyne-modified molecule via a copper-catalyzed azide-alkyne cycloaddition (CuAAC) or a strain-promoted azide-alkyne cycloaddition (SPAAC), creating a stable covalent bond.

In FIG. 13A, for a “C-terminal sortase tag” conjugation, for example, for sortase-mediated conjugation, a VHH tagged with a C-terminal LPXT/G₂ is incubated with Sortase A and pentaglycine-functionalized LNPs. Sortase A cleaves between the threonine and glycine residues of the LPXTG₂ motif (wherein, X is any amino acid), enabling site-specific conjugation of VHH to the LNP via an amide bond formation with the pentaglycine moiety.

In FIG. 13A, for “C-terminal transglutaminase” conjugation, for example, in transglutaminase-mediated conjugation, a VHH engineered with a C-terminal GQQQLG tag serves as a substrate for microbial transglutaminase (mTG). The enzyme catalyzes the formation of an amide bond between the glutamine residue in the tag and a primary amine-functionalized LNP, enabling site-specific and stable conjugation under mild reaction conditions.

FIG. 13B shows flow cytometry data showing eGFP positive cells in conjugates using “C-terminal cysteine” conjugation using thiol-maleimide and “C-terminal azide” conjugation using azide-dibenzocyclooctyne (DBCO) conjugations relative to an unconjugated Base LNP control. The results showed that both conjugation chemistries were successful and resulted in comparable expression of the eGFP protein.

FIG. 13C shows flow cytometry data showing mGreenLantern positive cells in conjugates using “C-terminal sortase tag” sortase-catalyzed conjugation (e.g., sortase-pentaglycine conjugation; two replicates of sortase-catalyzed conjugations are shown). The results showed that sortase-catalyzed conjugation were successful and resulted in expression of mGL protein. Overall, the results from this study showed various conjugation chemistries showed successful conjugation of the VHH319 to the lipid nanoparticle as demonstrated by expression of eGFP or mGL reporters.

### Example 14: Higher Anti-CD19 Expression and Reporter (mGL) Expression of circRNA Relative to Linear RNA

### A. CircRNA Increased Reporter Expression Relative to Linear RNA in Immune Cells

This example illustrates gene expression of a reporter gene, mGreenLantern, from circRNAs (encoding mGreenLantern) relative to linear RNA in immune cells.

In this example, 0.25 mg/kg or 0.5 mg/kg circRNA (both linear or circRNA) encapsulated in LNP comprising Compound 2 was administered intravenously to mice, and the bone marrow, lung, lymph nodes, peripheral blood, and spleen of the mice were harvested at 24 hours post RNA-LNP administration. Cells were gated as CD45.2+ and analyzed by flow cytometry. Gene (mGL) expression was measured. Of the tissues analyzed, bone marrow had the highest expression followed by the lungs, peripheral blood, and spleen.

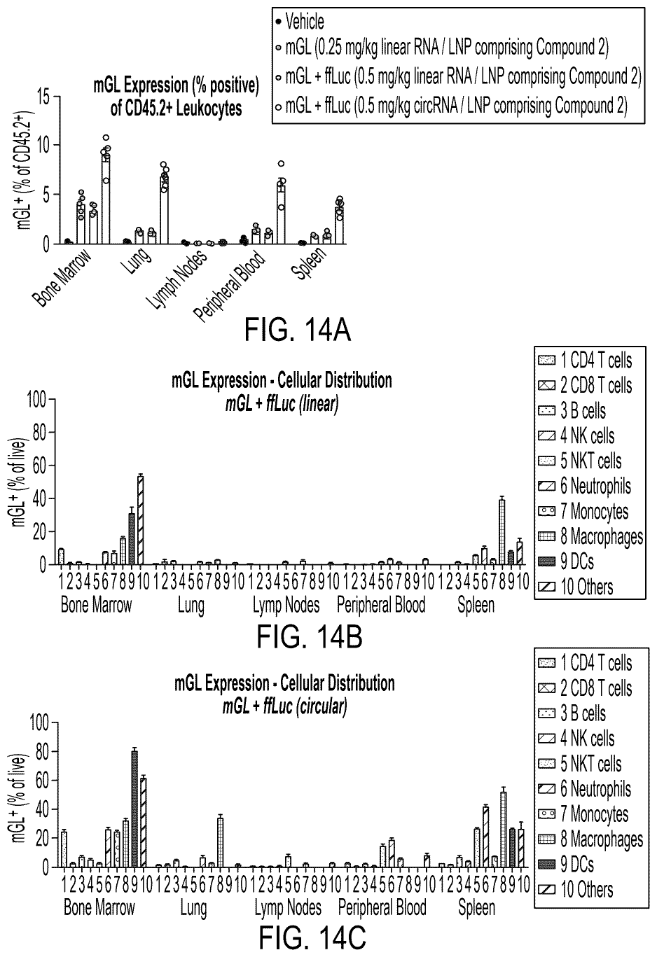

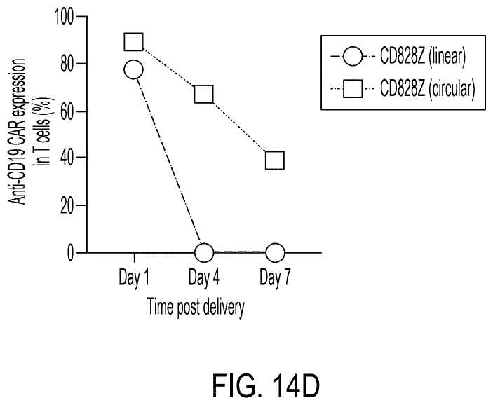

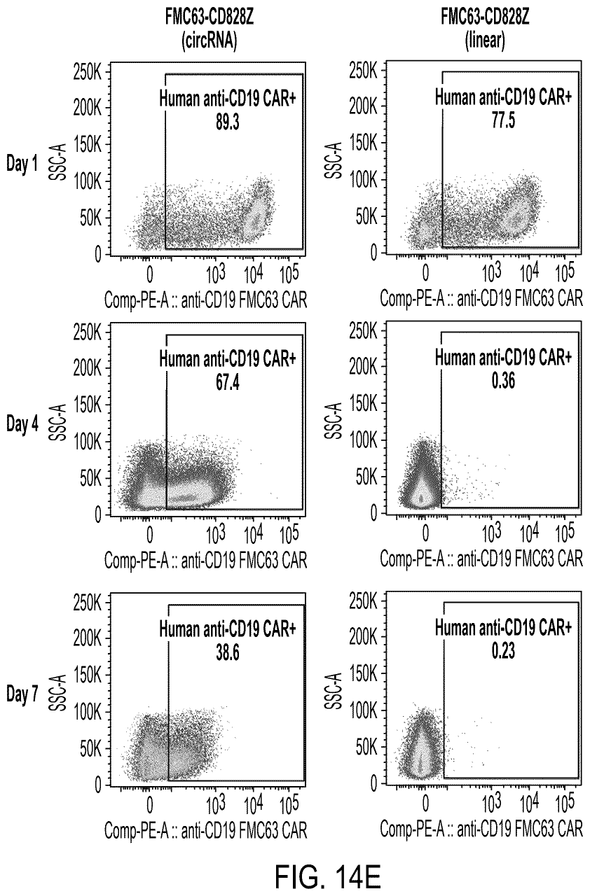

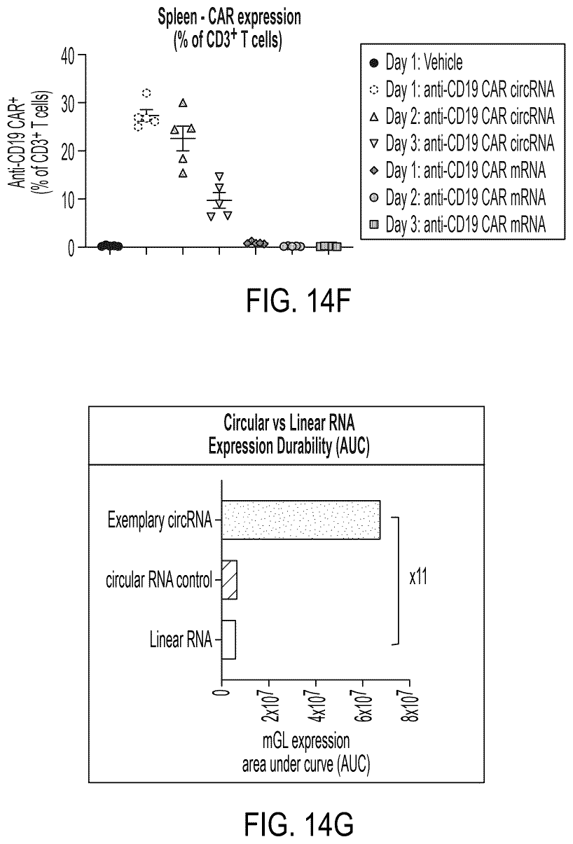

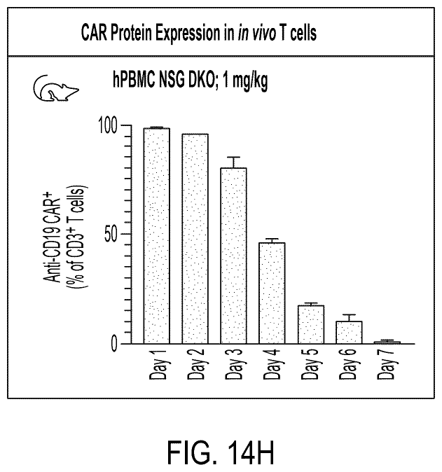

FIG. 14A is a graph of mGL positive cells as a percentage of total CD45.2+ leukocytes. The results shown in FIG. 14A showed that circular RNA increased mGL expression in all tissues that had detectable mGL signal, by at least about 4% or more of CD45.2⁺ leukocytes.

Overall, this study showed that reporter expression from circular RNA was higher than reporter expression from linear RNA in immune cells.

### B. CircRNA Increased Reporter Expression Relative to Linear RNA in Several Types of Immune Cells

This example illustrates that circRNA increased gene expression of a reporter gene relative to linear RNA in different types of immune cells.

Bone marrow, lung, lymph nodes, peripheral blood, and spleen were harvested at 24 hours post RNA-LNP administration. Cells were gated as CD45.2+. Then, each cell subset was gated based on their cell-type specific marker.

FIG. 14B is a graph of cellular biodistribution of gene expression in cells administered 0.5 mg/kg of linear RNA in an LNP comprising ionizable lipid Compound 2. FIG. 14C is a graph of cellular biodistribution of gene expression in cells administered 0.5 mg/kg of circular RNA in an LNP comprising ionizable lipid Compound 2.

The results in FIG. 14B and FIG. 14C showed that mGL expression was observed in bone marrow and spleen, which indicated that gene expression in these tissues was achieved through both linear and circular RNA, although the level of expression from circular RNA was higher.

FIG. 14C also showed detectable mGL expression in lung, lymph nodes, and peripheral blood, which indicated that circRNA achieved higher gene expression in these tissues. Further, FIG. 14C showed that the cellular biodistribution pattern was similar in the circRNA group in a given tissue sample.

Overall, this example showed that circRNA (encoding a reporter protein) increased reporter expression relative to linear RNA (encoding a reporter protein) in different types of immune cells.

### C. CircRNA Showed Increased Durability of CAR Expression Relative to Linear RNA in Activated T Cells

This example illustrates increased durability of CAR expression of circRNA relative to linear RNA in vitro in activated human T cells. The RNA was not encapsulated in a LNP in this example.

FIG. 14D is a graph of anti-CD19 CAR expression in T cells derived from flow cytometry data shown in FIG. 14E on day 1, 4 and 7 comparing expression from linear RNA (SEQ ID NO: 274) and circRNA (SEQ ID NO: 230).

FIG. 14E is flow cytometry data from day 1, day 4, and day 7 comparing anti-CD19 CAR positive cells from linear RNA (SEQ ID NO: 274) and circRNA (SEQ ID NO: 230).

As shown in FIG. 14D, at day 1, anti-CD19 CAR expressed from linear RNA (“FMC63-CD828Z (linear)”) showed about 80% anti-CD19 CAR expression. In contrast, at day 1, anti-CD19 CAR expressed from circRNA (“FMC63-CD828Z (circular)”) showed higher expression of about 90% in T cells. At day 4, anti-CD19 CAR from circRNA showed about 70% expression in T cells, while there was no detectable expression from linear RNA. At day 7, anti-CD19 CAR from circRNA showed about 40% expression in T cells, while there was no detectable expression from linear RNA.

Overall, the results from this study showed that circRNA (encoding anti-CD19 CAR) showed higher and more durable anti-CD19 expression than its linear RNA counterpart in vitro in activated human T cells.

### D. CircRNAs Showed Increased and Sustained Anti-CD19 CAR Expression Relative to Linear RNAs in Spleen In Vivo

This example illustrates higher anti-CD19 CAR expression from circRNA relative to linear RNA in the spleen of hCD34+ humanized mice.

Humanized hCD34+ mice were administered intravenously 1 mg/kg circRNAs or linear RNAs encoding anti-CD19 CAR (e.g., circRNA comprising a sequence of SEQ ID NO: 175, or linear mRNA comprising a sequence of SEQ ID NO: 275), and evaluated for anti-CD19 CAR expression in the spleen at day 1, day 2 and day 3 after administration. The circRNA was encapsulated in LNP-2 (as described in Table 16), which was conjugated to anti-CD5 targeting moiety VHH319 via a thiol-maleimide conjugation using the cysteine in the conjugation handle of SEQ ID NO: 120.

FIG. 14F is a graph of anti-CD19 CAR expression (as a percentage of CD3+ T cells) in the spleen of humanized CD4+ mice on day 1, day 2, and day 3, and compared expression from linear RNA and circRNA. As seen from FIG. 14F, anti-CD19 CAR expression was higher from the circRNA construct on day 1, day 2 and day 3 compared to vehicle control and also compared to the linear RNA construct, which showed comparable expression to the vehicle control.

The results from this study showed that circRNA encoding anti-CD19 CAR showed higher and more durable expression than its linear RNA counterpart in vivo in humanized mice.

### E. CircRNAs Showed Higher CAR Expression and Sustained Higher CAR Protein Production Over Time Relative to Linear RNAs.

This example illustrates higher anti-CD19 CAR expression from circRNA in primary human T cells in vitro as well as sustained CAR protein production in vivo.

FIG. 14G is a plot showing area under the curve (AUC) for mGreenLantern (mGL) reporter protein expression by circRNAs comprising an exemplary IRES and circRNA comprising a reference IRES sequence (“circular RNA control”), compared to a linear RNA encoding mGL reporter protein in activated primary human T cells (from one donor). More specifically, the exemplary circRNA comprised IRES644, and the circular RNA control comprised IRES090. In this study, following T-cell activation, RNAs were electroporated into cells at a dose of 250 ng/1×10⁶ cells, and mGL expression was monitored via Incucyte for 88 hours (approximately 3.5 days). As shown in FIG. 14G, the circRNA comprising an exemplary IRES showed reporter protein expression (expressed as AUC) that was approximately 11 times greater than that of either the circular RNA control or the linear RNA.

### F. CircRNAs Showed Sustained CAR Protein Expression In Vivo

In an in vivo study, mice were administered circRNA encoding anti-CD19 CAR formulated in a targeted LNP, and CAR expression was evaluated over time. On day −14, mice were engrafted with human PBMCs (hPBMCs) from a single donor. On day −4 prior to dosing, mice were randomized based on the levels of human B and T cells in the blood evaluated by flow cytometry after confirming hPBMC engraftment. Upon confirmation, they were randomly assigned into study groups. On day 0, mice were dosed with circRNA encoding anti-CD19 CAR comprising a sequence of SEQ ID NO: 175 containing IRES263 formulated in an anti-CD5 targeted LNP (LNP-18 as described in Table 16), or vehicle (diluent used for LNP groups) via tail vein injection at 1.0 mg/kg dose (100 L/mouse). On each day up to 7 days, anti-CD19 CAR expression was measured in CD3⁺ T cells from spleen using flow cytometry. The results are shown in FIG. 14H.

FIG. 14H is a graph that showed a time course of anti-CD19 CAR+ T cells measured over 7 days in the mice administered 1 mg/kg of circRNA encoding anti-CD19 CAR formulated in a targeted LNP (comprising Compound 7), which demonstrated that anti-CD19 CAR+ T cell populations were sustained in vivo.

In summary, the results shown in FIG. 14G demonstrated that the exemplary circRNA improved reporter gene expression compared to the circular RNA control and linear RNA in activated primary human T cells. Further, the results in FIG. 14H demonstrated that CAR+ T cell populations were maintained in vivo, which resulted in sustained and durable CAR expression, and supported extended therapeutic windows and less frequent dosing of the circRNA.

### Example 15: In Vivo Administration of circRNA (Encoding for Anti-CD19 CAR) Conjugated to Anti-CD5 Targeted LNP in Humanized Mice Showed T Cell Activation and Deep B Cell Depletion

CD34+ HSC engrafted humanized mice were administered intravenously circRNA encapsulated by LNP conjugated to anti-CD5 targeting moiety VHH319 or vehicle control (diluent only) via tail vein injection at 0.5 mg/kg of the circRNA on days 0, 7, and 14. CAR expression was measured in T cells from the peripheral blood and spleen of the humanized mice as measured 24 hours after administration of each dose, on day 1, 8, and 15.

The circRNA administered in this Example comprised a sequence of SEQ ID NO: 264. The circRNA comprises a 5′ UTR; an IRES of SEQ ID NO: 1; a coding sequence encoding a CAR comprising an anti-CD19 scFv FMC63 (having a VL sequence of SEQ ID NO: 54, linked by a linker having the amino acid sequence of SEQ ID NO: 56, and a VH sequence of SEQ ID NO: 55), a CD8a hinge, a CD8a transmembrane domain, a 4-1BB costimulatory domain, a CD3 zeta signaling domain, and a CD8 signal peptide; a 3′ UTR comprising one microRNA binding site, specifically, a miR-122 binding site that comprises SEQ ID NO: 267. An anti-CD5 targeting moiety VHH319 (SEQ ID NO: 89) with the conjugation handle of SEQ ID NO: 120 attached at the C terminus (the VHH with conjugation handle having the sequence of SEQ ID NO: 122) was conjugated to an LNP via thiol-maleimide conjugation using the cysteine in the conjugation handle. The LNP composition was LNP-18 as described in Table 16, and the ionizable lipid was Compound 7.

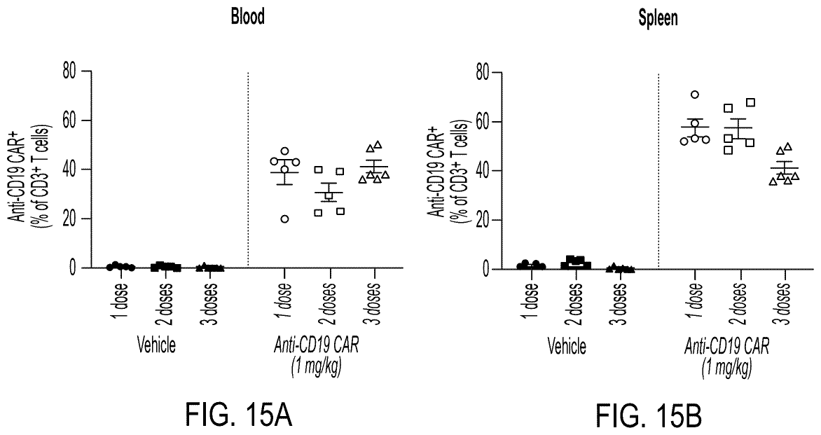

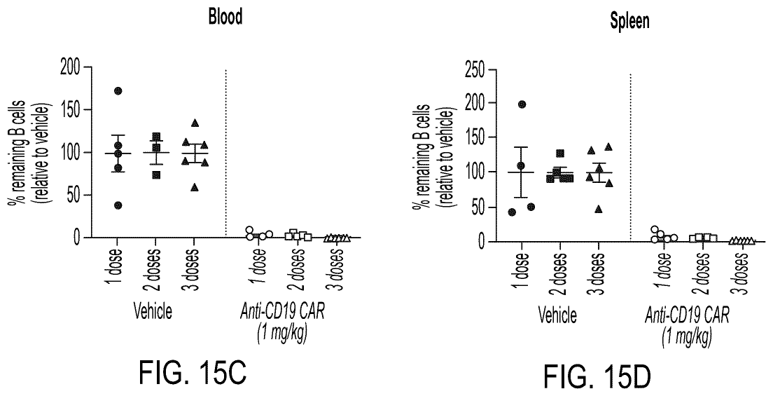

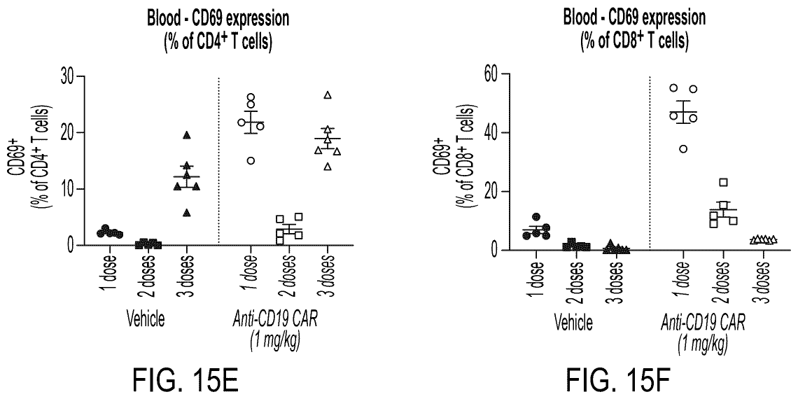

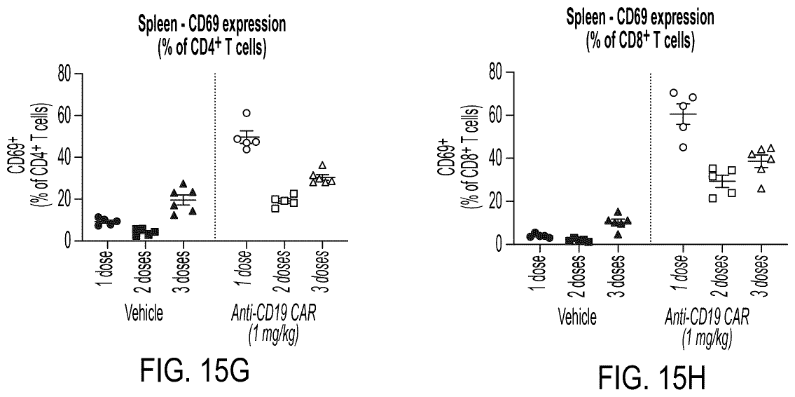

FIG. 15A is a graph of percent anti-CD19 CAR positive cells as a percentage of total CD3 positive T cells in peripheral blood, 24 hours after administration of each of three doses of circRNA (encoding for anti-CD19 CAR) conjugated to targeted LNP relative to vehicle control.

FIG. 15B is a graph of percent anti-CD19 CAR positive cells as a percentage of total CD3 positive T cells in spleen, 24 hours after administration of each of three doses of circRNA (encoding for anti-CD19 CAR) conjugated to targeted LNP relative to vehicle control.

For FIG. 15A and FIG. 15B, the results for each treatment condition represented the mean±SEM (standard error of the mean) of five different mice (N=5) (one-way ANOVA, Tukey's test for multiple comparisons was used). The results in FIG. 15A and FIG. 15B showed that anti-CD19 CAR⁺ CD3⁺ T cells were successfully generated at a similar level after each of the three doses of circRNA (encoding for anti-CD19 CAR) conjugated to targeted LNP administered, both in the blood and spleen of mice relative to vehicle control.

FIG. 15C is a graph of percent remaining B cells (relative to vehicle control) in blood after administration of three doses each of 0.5 mg/kg circRNA in humanized mice, as measured 24 hours after administration of each dose, on day 1, 8 and 15.

FIG. 15D is a graph of percent remaining B cells (relative to vehicle control) in spleen after administration of three doses each of 0.5 mg/kg circRNA in humanized mice, as measured 24 hours after administration of each dose, on day 1, 8 and 15.

For FIG. 15C and FIG. 15D, the amount of B cell depletion was calculated as percentage of remaining B cells in peripheral blood and spleen. For each dosing regimen, B cell counts from each treated mouse were divided by the average of corresponding vehicle treated mice followed by multiplication with 100 to calculate percentage of remaining B cells relative to vehicle after every dose. The results for each treatment condition represented the mean±SEM of five different mice (N=5).

The results in FIG. 15C and FIG. 15D showed that robust B cell depletion was seen in both the blood and spleen of humanized mice after administration of a single dose of circRNA, and administration of additional doses of circRNA further augmented B cell depletion with each successive dose in both the blood and spleen. Notably, after the administration of a third dose of circRNA, the treated mice showed almost complete B cell depletion in both peripheral blood and spleen.

FIG. 15E is a graph of percent CD69-positive CD4+ T cells in blood 24 hours after intravenous administration of each of three doses (1 mg/kg) of circRNA.

FIG. 15F is a graph of percent CD69-positive CD8+ T cells in blood 24 hours after intravenous administration of each of three doses (1 mg/kg) of circRNA.

FIG. 15G is a graph of percent CD69-positive CD4+ T cells in spleen 24 hours after intravenous administration of each of three doses (1 mg/kg) of circRNA.

FIG. 15H is a graph of percent CD69-positive CD8+ T cells in spleen 24 hours after intravenous administration of each of three doses of (1 mg/kg) of circRNA.

CD69 is an early activation marker for T cells, i.e., it is one of the first surface proteins expressed on a T cell when it becomes activated by an antigen, rapidly appearing on the cell membrane shortly after T cell receptor (TCR) engagement, indicating T cell activation. The results in FIG. 15E-FIG. 15H showed T cell activation in the blood and spleen of humanized mice after circRNA-LNP administration. The results for each treatment condition represented the mean±SEM of five different mice (N=5).

Overall, these results showed that administration of one and multiple doses of the circRNA (encoding anti-CD19 CAR) encapsulated by LNP comprising Compound 7 and conjugated to anti-CD5 targeting moiety VHH319 increased functional efficacy in vivo in humanized mice, as shown by both T cell activation and significant depletion of B cells.

### Example 16: Pharmacokinetic and Pharmacodynamic Study of circRNA (Encoding for Anti-CD19 CAR) Conjugated to Anti-CD5 Targeted LNP in a CD34+ NSG Mouse Model

In this study, CD34+ HSC-engrafted NSG mice were administered intravenously 0.5 mg/kg of circRNA (SEQ ID NO: 264), which was encapsulated in LNP that was conjugated to anti-CD5 targeting moiety VHH319. The circRNA comprises a sequence of SEQ ID NO: 264. The LNP composition was LNP-18, and comprises ionizable lipid Compound 7. The circRNAs formulated in LNP conjugated to anti-CD5 targeting moiety VHH319 that was tested in Example 16 was the same as the RNA-LNP tested in Example 15.

The expression kinetics of the circRNA (encoding anti-CD19 CAR) conjugated to anti-CD5-targeted LNP and efficacy of B cell depletion were evaluated at day 1, 2, and 7 post injection. Further, expression and efficacy were evaluated in a dose curve with administration of 0.1, 0.5, and 1 mg/kg of the circRNA. Mice were administered with the circRNA-LNP or vehicle (diluent only) via tail vein injection at 0.1 mg/kg, 0.5 mg/kg, or 1.0 mg/kg on day 0. CAR expression was measured by flow cytometry and corresponding B cell depletion was evaluated at 24 hours after administration, on day 1, 2, and 7.

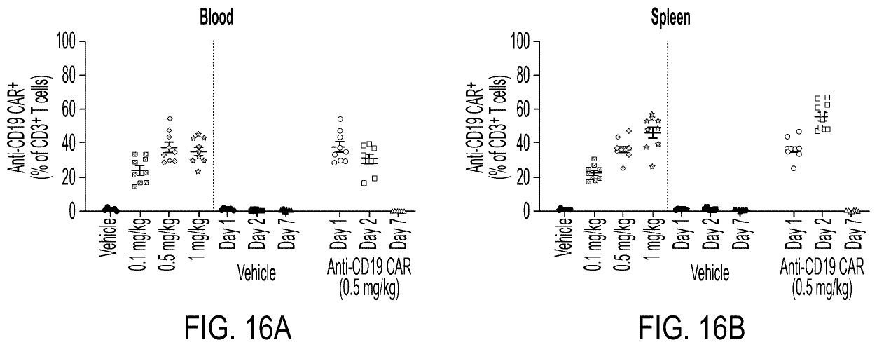

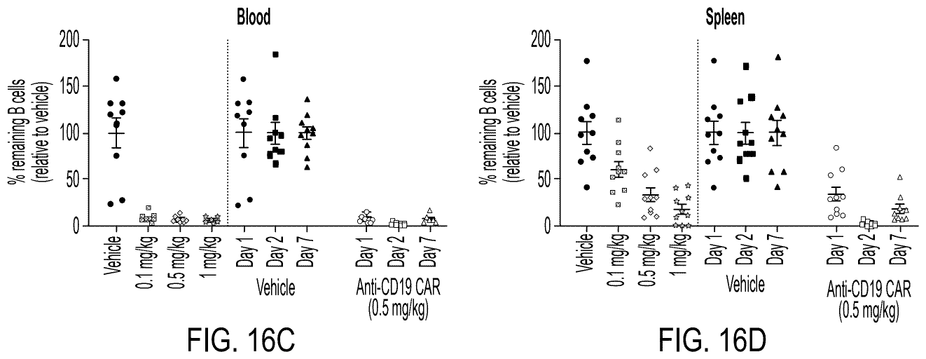

FIG. 16A is a graph of percent anti-CD19 CAR positive cells as a percentage of total CD3 positive T cells in blood. The left panel shows anti-CD19 CAR positive cells, 24 hours after administration of 0.1 mg/kg, 0.5 mg/kg, or 1.0 mg/kg doses of the circRNA encoding for anti-CD19 CAR relative to vehicle control. The right panel shows anti-CD19 CAR positive cells, 24 hours after administration of 0.5 mg/kg dose of circRNA (encoding anti-CD19 CAR) relative to vehicle control on days 1, 2 and 7 after administration.

FIG. 16B is a graph of percent anti-CD19 CAR positive cells as a percentage of total CD3 positive T cells in spleen. The left panel shows anti-CD19 CAR positive cells, 24 hours after administration of 0.1 mg/kg, 0.5 mg/kg or 1.0 mg/kg doses of circRNA (encoding for anti-CD19 CAR) to vehicle control. The right panel shows anti-CD19 CAR positive cells, 24 hours after administration of 0.5 mg/kg dose of circRNA (encoding for anti-CD19 CAR) relative to vehicle control on days 1, 2 and 7 after administration.

FIG. 16C is a graph of percent remaining B cells (relative to vehicle control) in blood. The left panel shows percent remaining B cells, 24 hours after administration of 0.1 mg/kg, 0.5 mg/kg and 1.0 mg/kg doses of circRNA (encoding for anti-CD19 CAR) relative to vehicle control. The right panel shows percent remaining B cells, 24 hours after administration of 0.5 mg/kg dose of circRNA (encoding for anti-CD19 CAR) relative to vehicle control on days 1, 2 and 7 after administration.

FIG. 16D is a graph of percent remaining B cells (relative to vehicle control) in spleen. The left panel shows percent remaining B cells, 24 hours after administration of 0.1 mg/kg, 0.5 mg/kg, or 1.0 mg/kg doses of circRNA (encoding for anti-CD19 CAR) relative to vehicle control. The right panel shows percent remaining B cells, 24 hours after administration of 0.5 mg/kg dose of circRNA (encoding for anti-CD19 CAR) relative to vehicle control on days 1, 2 and 7 after administration.

The results in FIG. 16A-16B showed that CD3⁺ T cells showed dose-dependent anti-CD19 CAR expression both in peripheral blood and spleen in a CD34+ NSG mouse model. CAR expression was detected both on day 1 and day 2, while it was undetectable by day 7 post dose. The results for each treatment condition represented the mean±SEM of ten different mice (N=10).

Further, the results in FIG. 16C-FIG. 16D showed that the circRNA (encoding for anti-CD19 CAR) formulated in LNP conjugated to anti-CD5 targeting moiety VHH319 that was administered in vivo to humanized mice was both tolerated and efficacious at depleting B cells at all doses tested. The amount of B cell depletion was calculated as percentage of remaining B cells in peripheral blood and spleen. For each dosing regimen, B cell counts from each treated mouse were divided by the average of corresponding vehicle-treated mice followed by multiplication with 100 to calculate the percentage of remaining B cells relative to vehicle after every dose. The results for each treatment condition represented the mean±SEM of ten different mice (N=10).

### Example 17: Pharmacokinetic and Pharmacodynamic Study of circRNA (Encoding Anti-CD19 CAR)-Targeted LNP in a Humanized PBMC-Engrafted NSG MHC-I/II Double Knockout (DKO) Mouse Model

In this example, humanized PBMC-engrafted NSG DKO mice were administered intravenously circRNA (encoding anti-CD19 CAR) formulated in LNP conjugated to anti-CD5 targeting moiety VHH319 or vehicle control (diluent only) via tail vein injection at 0.1 mg/kg, 0.5 mg/kg, or 1.0 mg/kg circRNA on day 0. In the humanized PBMC NSG MHC-I/II DKO mouse model, healthy human peripheral blood mononuclear cells (PBMCs) were engrafted via intravenous injection to create a pseudo-human immune system. The inactivation of the MHC-I/II prevents the possibility of developing Graft-versus-host disease (GvHD). In this model, T cells display an effector memory or effector phenotype similar to activated T cells, while B cells show robust CD19 expression similar to what has been observed in the CD34⁺ NSG model. The PBMC NSG DKO model has a ratio of T cells and B cells similar to those observed in the human blood and spleen.

Anti-CD19 CAR expression and efficacy of B cell depletion were evaluated at day 1, 2 and 7 post injection. The circRNA comprises a sequence of SEQ ID NO: 264. The LNP composition was LNP-18. The circRNAs formulated in LNP conjugated to anti-CD5 targeting moiety VHH319 that was tested in Example 17 was the same as the RNA-LNP tested in Examples 15 and 16. On day 1, 2, and 7, CAR expression was measured in T cells from blood and spleen of the humanized mice using flow cytometry, and the proportion of B cells was measured in the blood and spleen and compared to vehicle-injected mice.

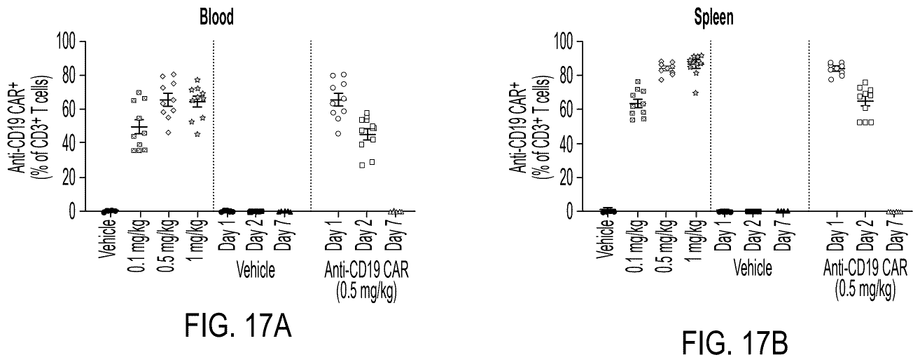

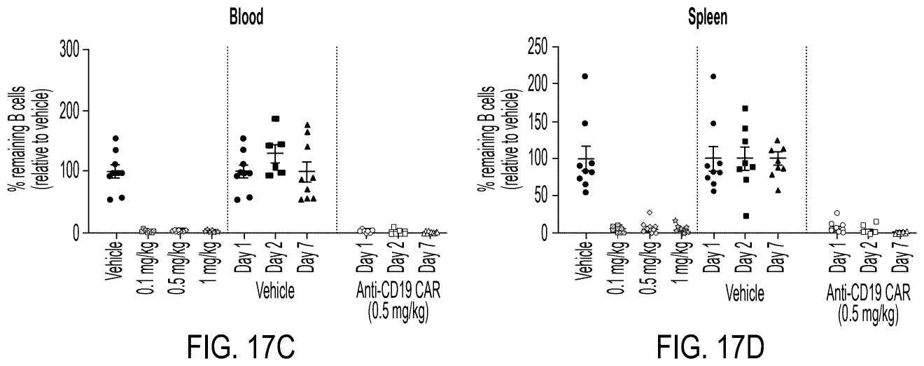

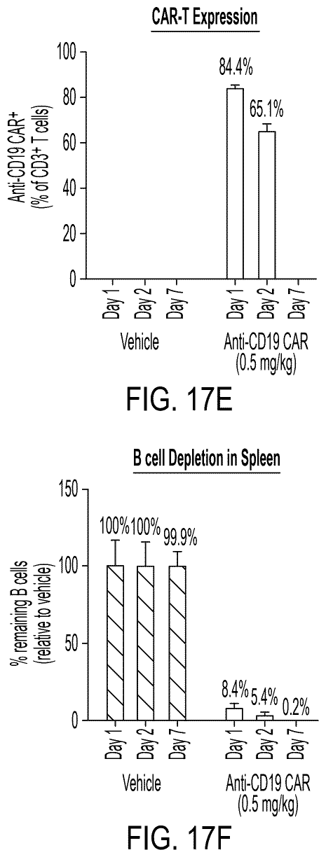

FIG. 17A is a graph of percent anti-CD19 CAR positive cells as a percentage of total CD3 positive T cells in blood. The left panel shows anti-CD19 CAR positive cells, 24 hours after administration of 0.1 mg/kg, 0.5 mg/kg and 1.0 mg/kg doses of circRNA (encoding anti-CD19 CAR) relative to vehicle control. The right panel shows anti-CD19 CAR positive cells, 24 hours after administration of 0.5 mg/kg dose of circRNA (encoding anti-CD19 CAR) relative to vehicle control on days 1, 2 and 7 after administration.

FIG. 17B is a graph of percent anti-CD19 CAR positive cells as a percentage of total CD3 positive T cells in spleen. The left panel shows anti-CD19 CAR positive cells, 24 hours after administration of 0.1 mg/kg, 0.5 mg/kg, or 1.0 mg/kg doses of circRNA (encoding anti-CD19 CAR) relative to vehicle control. The right panel shows anti-CD19 CAR positive cells, 24 hours after administration of 0.5 mg/kg dose of circRNA (encoding anti-CD19 CAR) relative to vehicle control on days 1, 2 and 7 after administration.

FIG. 17C is a graph of percent remaining B cells (relative to vehicle control) in blood. The left panel shows percent remaining B cells, 24 hours after administration of 0.1 mg/kg, 0.5 mg/kg, or 1.0 mg/kg doses of circRNA (encoding anti-CD19 CAR) relative to vehicle control. The right panel shows percent remaining B cells, 24 hours after administration of 0.5 mg/kg dose of circRNA (encoding anti-CD19 CAR) relative to vehicle control on days 1, 2 and 7 after administration.

FIG. 17D is a graph of percent remaining B cells (relative to vehicle control) in spleen. The left panel shows percent remaining B cells, 24 hours after administration of 0.1 mg/kg, 0.5 mg/kg, or 1.0 mg/kg doses of circRNA (encoding anti-CD19 CAR) relative to vehicle control. The right panel shows percent remaining B cells, 24 hours after administration of 0.5 mg/kg dose of circRNA (encoding anti-CD19 CAR) relative to vehicle control on days 1, 2 and 7 after administration.

The results in FIG. 17A-FIG. 17B showed that CD3⁺ T cells showed dose-dependent CAR expression both in the peripheral blood and spleen of the humanized mice. CAR expression was detected both on day 1 and day 2, while it was undetectable by day 7 post dose. For both FIG. 17A and FIG. 17B, the results for each treatment condition represented the mean±SEM of ten different mice (N=10).

The results in FIG. 17C-FIG. 17D showed that the amount of B cell depletion was substantial on day 1 post dose (about 96% B cell depletion in the blood, and 92% B cell depletion in the spleen at 0.5 mg/kg dose), and this substantial B cell depletion was maintained at least up to 7 days post administration of the circRNA. For FIG. 17C-17D, the amount of B cell depletion was calculated as percentage of remaining B cells in peripheral blood and spleen. For each time point, B cell counts from each treated mouse were divided by the average of corresponding vehicle treated mice followed by multiplication with 100 to calculate percentage of remaining B cells relative to vehicle. Data for each treatment condition represent the mean±SEM of ten different mice (N=10).

FIG. 17E is a bar graph shown as an alternate representation of the right panel of FIG. 17B. FIG. 17E shows anti-CD19 CAR positive cells (as a percentage of CD3+ T cells), 24 hours after administration of 0.5 mg/kg dose of circRNA (encoding anti-CD19 CAR) formulated in targeted LNP relative to vehicle control on days 1, 2 and 7 after administration.

FIG. 17F is a bar graph shown as an alternate representation of FIG. 17D. FIG. 17F shows percent remaining B cells (relative to vehicle) after B cell count was normalized to total live splenocytes, 24 hours after administration of 0.5 mg/kg dose of circRNA (encoding anti-CD19 CAR) formulated in targeted LNP relative to vehicle control on days 1, 2 and 7 after administration.

Overall, this study showed that CAR expression on T cells was dose-dependent and led to substantial B cell depletion as early as day 1 following administration of the circRNA (encoding anti-CD19 CAR)-LNP conjugated to an anti-CD5 targeting moiety VHH319, and the B cell depletion was maintained for at least up to day 7 after administration.

### Example 18: In Vivo Mice Study Demonstrated Robust Delivery of circRNA (Encoding Anti-CD19 CAR) to the White Pulp of the Spleen

The goal of this study was to use in situ hybridization (ISH) to evaluate successful delivery of circRNAs (encoding anti-CD19 CAR) to the white pulp of spleen. On day 5 prior to dosing, CD34+ NSG mice were randomized based on the levels of B cells and T cells in the blood evaluated by flow cytometry, and then they were randomly assigned into study groups. The mice were dosed with 1.0 mg/kg circRNA (100 μL/mouse) formulated in anti-CD5 targeted LNP or vehicle (diluent used for LNP groups) via tail vein injection. The circRNA encoding anti-CD19 formulated in LNP conjugated to anti-CD5 targeting moiety VHH319 that was tested in this Example was the same as the RNA-LNP tested in Example 15.

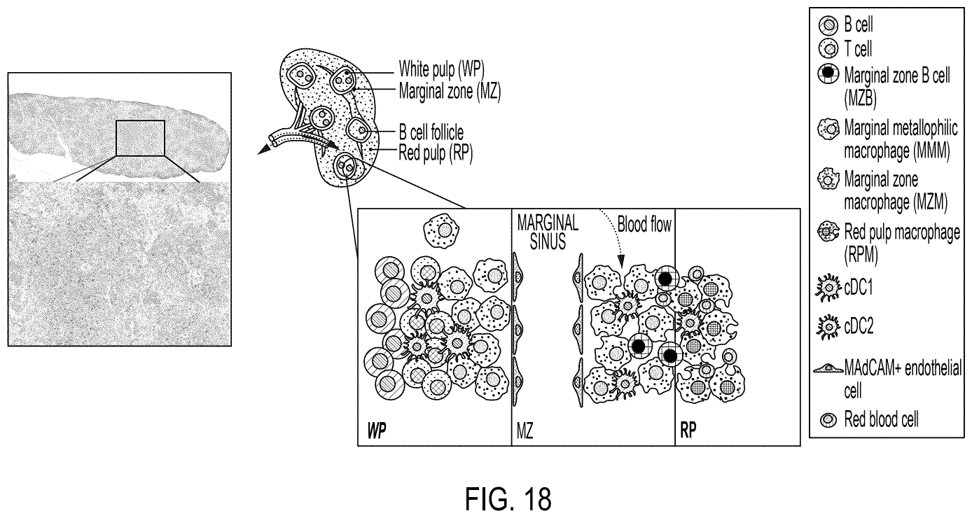

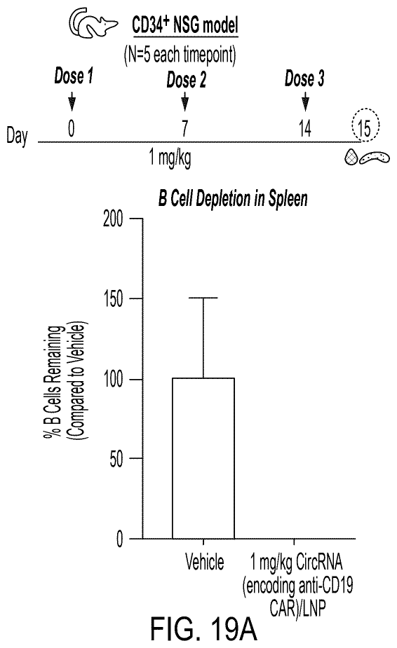

At 4 hours post dose, the mice were euthanized, and CAR-T delivery was assessed using ISH with probes against the anti-CD19 CAR. As shown in FIG. 18, the data demonstrated robust delivery of the circRNA to the white pulp of the spleen, which is the location of splenic T cell, and was the targeted site of delivery. In other words, this Example showed selective localization of the circRNA (encoding anti-CD19 CAR) to T cell-rich zones, which is important for effective in vivo CAR-T cell generation.

### Example 19: In Vivo Mice Study Showed that Administration of circRNA (Encoding Anti-CD19 CAR) Showed Robust Delivery to the White Pulp of the Spleen and Robust B Cell Depletion in the Spleen

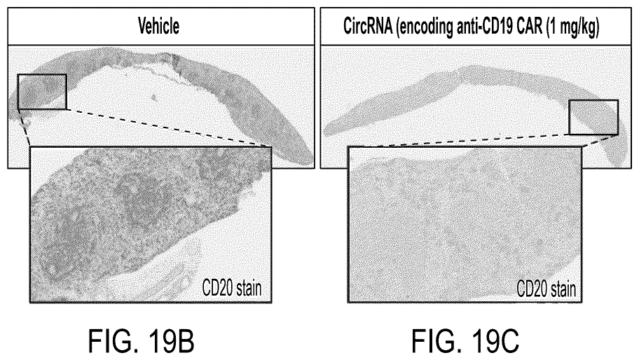

The goal of this study was to evaluate B cell depletion using immunohistochemistry (IHC) after administration of circRNA (encoding anti-CD19 CAR). In brief, the study evaluated repeat dosing with circRNA (encoding anti-CD19 CAR) formulated in anti-CD5 targeted LNP. The circRNA encoding anti-CD19 formulated in LNP conjugated to anti-CD5 targeting moiety VHH319 that was tested in this Example was the same as the RNA-LNP tested in Example 15. CD34+ NSG mice were dosed with 1.0 mg/kg circRNA (100 μL/mouse) or vehicle (diluent used for LNP groups) via tail vein injection on days 0, 7, and 14, as shown in FIG. 19A. On Day 15, mice from each group were euthanized, and CAR-T expression was measured in T-cells from blood (not shown) and spleen using flow cytometry, and the amount of B cell depletion was evaluated using IHC using a B-cell marker (via a CD20 stain). Efficacy was defined as a significant depletion of B cells in spleen, compared to mice injected with the vehicle control. FIG. 19A is a graph of the percentage of B cells remaining (compared to vehicle), and the results showed significant B cell depletion after administration of 1 mg/kg circRNA (encoding anti-CD19 CAR). In FIG. 19B and FIG. 19C, the dark staining in the vehicle in the white pulp of spleen showed a high density of B cells in the spleen (FIG. 19B). By contrast, FIG. 19C showed that administration of circRNA (encoding anti-CD19 CAR) demonstrated robust delivery of the circRNA (encoding anti-CD19 CAR) to the white pulp of the spleen. FIG. 19B and FIG. 19C showed that administration of circRNA encoding anti-CD19 CAR led to full B cell depletion in the spleen, as also confirmed by flow cytometry (FIG. 19A), all of which demonstrated potent in vivo functional activity of the circRNA.

### Example 20. A Two-Week Study of CircRNA (encoding Anti-CD20 CAR) in Targeted LNP Administered Intravenously to Cynomolgus Monkeys

In this Example, the objectives of this study were to determine the safety, tolerability, potency, and efficacy of circRNA encoding an anti-CD20 CAR formulated in targeted LNP intravenously administered to cynomolgus monkeys (non-human primates, “NHP”) for treating B-cell driven autoimmune diseases.

In one aspect, the CAR comprising an anti-CD20 antigen-binding fragment (an scFv) was a surrogate for a CAR comprising an anti-CD19 antigen-binding fragment. Similar to CD19, CD20 is also a B cell marker expressed on the surface of various B cells. Both CD19 and CD20 play a role in B cell signaling and activation. The anti-CD20 CAR was used because circRNA encoding an anti-CD19 CAR, as disclosed herein, is not cross-reactive between humans and cynomolgus monkeys. Specific differences exist between monkey and human B cell subsets in peripheral blood and lymphoid organs, including challenges for reagents to cross-react between human and non-human primate CD19 (Vugmeyster, 2004). There is a long history of successfully developing B cell-depleting therapeutics in cynomolgus monkeys by targeting CD20 (Schroder, 2003; Vugmeyster, 2003). For example, the first nonhuman primate model to evaluate CAR T on target function (B cell aplasia) as well as induction of safety signals was assessed via anti-CD20 CAR due to antigen expression compatibility of CD20 between NHP and humans, as well as CD19 cross-reactivity limitation (Taraseviciute et al, 2019). It has been demonstrated that an anti-CD20 CAR construct in the nonhuman primate model showed robust T cell CAR expression and resulted in significant B cell aplasia (Bui et al, 2024).

The circRNA administered in this Example comprises a 5′ UTR; an IRES of SEQ ID NO: 1; a coding sequence encoding an anti-CD20 scFv, a CD8a hinge (SEQ ID NO: 285), a CD8a transmembrane domain (SEQ ID NO: 287), a 4-1BB costimulatory domain (SEQ ID NO: 293), and a CD3 zeta signaling domain (SEQ ID NO: 294); and a 3′ UTR comprising one microRNA binding site, specifically, a miR-122 binding site that comprises SEQ ID NO: 267. The circRNA was formulated in targeted LNP, which was conjugated to anti-CD5 targeting moiety VHH319 (SEQ ID NO: 89) via thiol-maleimide conjugation using the cysteine in the conjugation handle (SEQ ID NO: 120). The LNP composition was LNP-18 as described in Table 16, and the ionizable lipid was Compound 7.

In this study, the cynomolgus monkeys were administered the circRNAs formulated in targeted LNPs via intravenous restrained peripheral infusion. Intravenous infusion was administered over 60 minutes using an infusion pump. A 0.5 mL flush of saline was administered post-infusion. The cynomolgus monkeys were not sedated during administration. As shown in Table 18, this study included 11 total groups with different dosing regimens: (1) two doses on Days 0 and 7 in Cohort 1; (2) two doses on Days 0 and 7 for groups 1-3, 5, and 7 in Cohort 2; and (3) one dose on Day 0 for groups 4 and 6 in Cohort 2. After the administration(s) of the circRNAs formulated in targeted LNPs, the organs (liver and spleen) and tissues (lymph nodes) were harvested at day 5, 8 or 15 as described in Table 18. The organs and tissues were analyzed for CAR expression levels and B cell depletion by flow cytometry. Blood was collected for hematological analysis at baseline, 6 hours, 24 hours, and 48 hours after each dose and at terminal. In addition, liver enzymes, cytokines, and complement were collected for hematological analysis as measures of safety and tolerability.

The experimental design of this NHP study is shown in Table 18 below.

TABLE 18
Experimental design of NHP study testing the safety, tolerability, potency, and
efficacy of circRNA encoding anti-CD20 CAR formulated in anti-CD5 targeted LNP.

| | | Test | | | | | |
|---|---|---|---|---|---|---|---|
| | | Article: | | | | | |
| | | CircRNA | | | | | |
| | | in targeted | Therapeutic | Infusion | Dose | | |
| | Group | LNP | Dose | Volume | Concentration | | Dosing |
| Cohort No. | No. | formulation | (mg/kg) | (mL/kg) | (mg/mL) | No. of Monkeys | Frequency |
| Cohort 1 | A | Vehicle | N/A | 10 | 0 | 1 (female) | Days 0 and |
| | | Control | | | | | 7 (necropsy |
| | | | | | | | on Day 15) |
| | B | Anti-CD20 | 1.0 | 10 | 0.1 | 2 | Days 0 and |
| | | CAR in | | | | (1 female, 1 male) | 7 (necropsy |
| | | anti-CD5 | | | | | on Day 15) |
| | C | targeted | 2.0 | 10 | 0.2 | 2 | Days 0 and |
| | | LNP | | | | (1 female, 1 male) | 7 (necropsy |
| | D | | 3.0 | 10 | 0.3 | 2 | on Day 8) |
| | | | | | | (1 female, 1 male) | |
| | | | | | | Total Monkeys in | |
| | | | | | | Cohort 1: 7 | |
| Cohort 2 | 1 | Vehicle | 0 | 10 | 0 | 1 (male) | Days 0 and |
| | | Control | | | | | 7 (necropsy |
| | | | | | | | on Day 8) |
| | 2 | Anti-CD20 | 0.25 | 10 | 0.025 | 1 (female) | Days 0 and |
| | | CAR in | | | | | 7 (necropsy |
| | | anti-CD5 | | | | | on Day 8) |
| | 3 | targeted | 0.5 | 10 | 0.05 | 1 (male) | Days 0 and |
| | | LNP | | | | | 7 (necropsy |
| | | | | | | | on Day 8) |
| | 4 | | 0.75 | 10 | 0.075 | 1 (female) | Day 0 |
| | | | | | | | (necropsy |
| | | | | | | | on Day 5) |
| | 5 | | 0.75 | 10 | 0.075 | 1 (male) | Days 0 and |
| | | | | | | | 7 (necropsy |
| | | | | | | | on Day 8) |
| | 6 | | 1.0 | 10 | 0.1 | 1 (female) | Day 0 |
| | | | | | | | (necropsy |
| | | | | | | | on Day 5) |
| | 7 | | 1.0 | 10 | 0.1 | 1 (male) | Days 0 and |
| | | | | | | | 7 (necropsy |
| | | | | | | | on Day 8) |
| | | | | | | Total Monkeys in | |
| | | | | | | Cohort 2: 7 | |

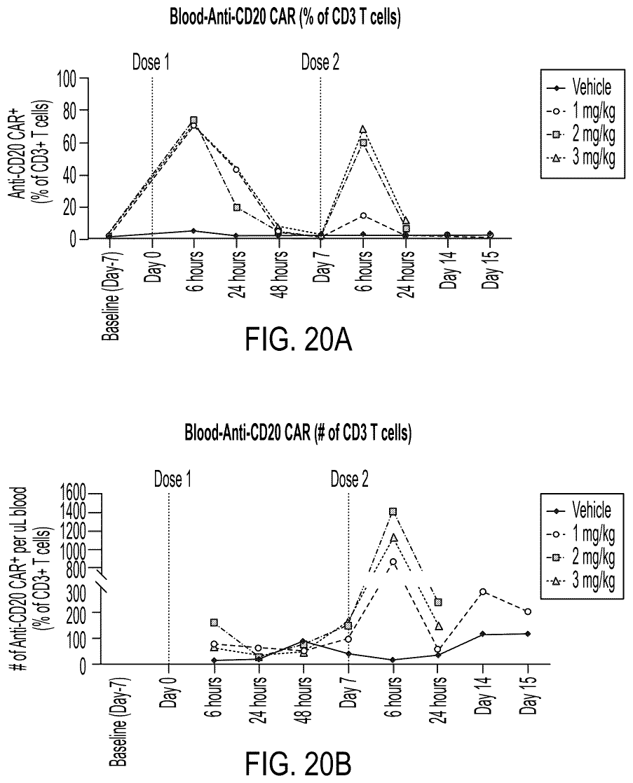

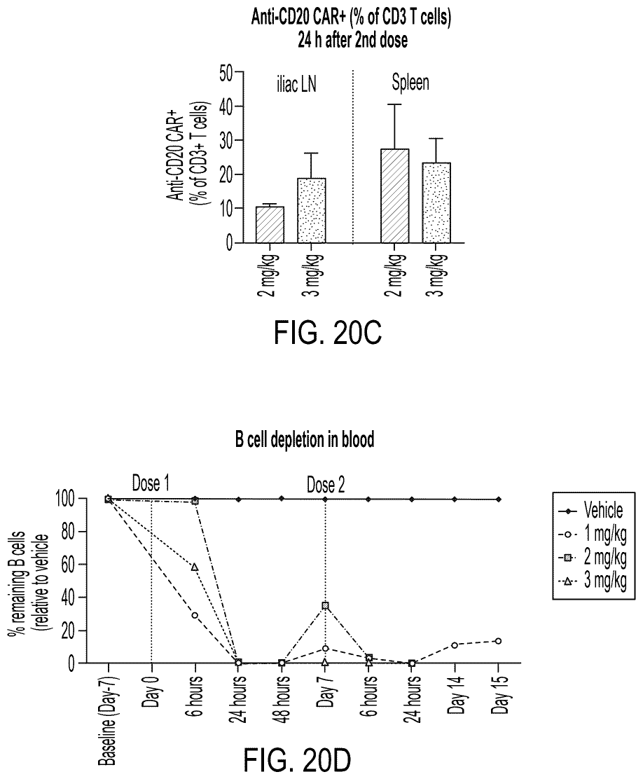

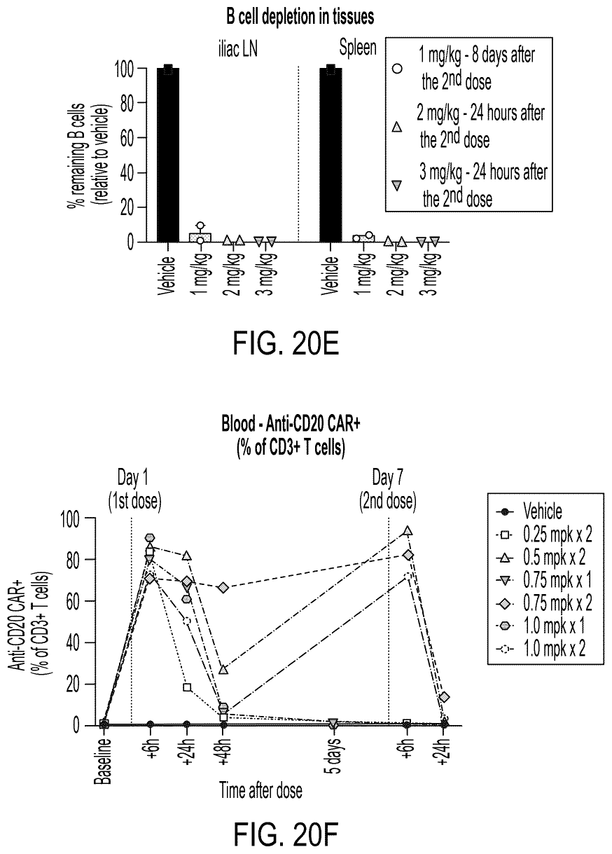

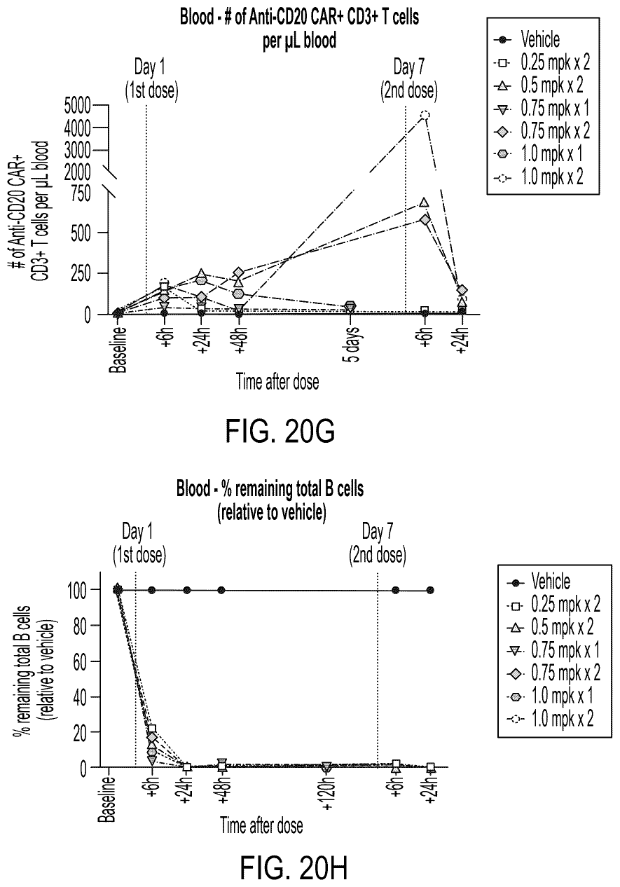

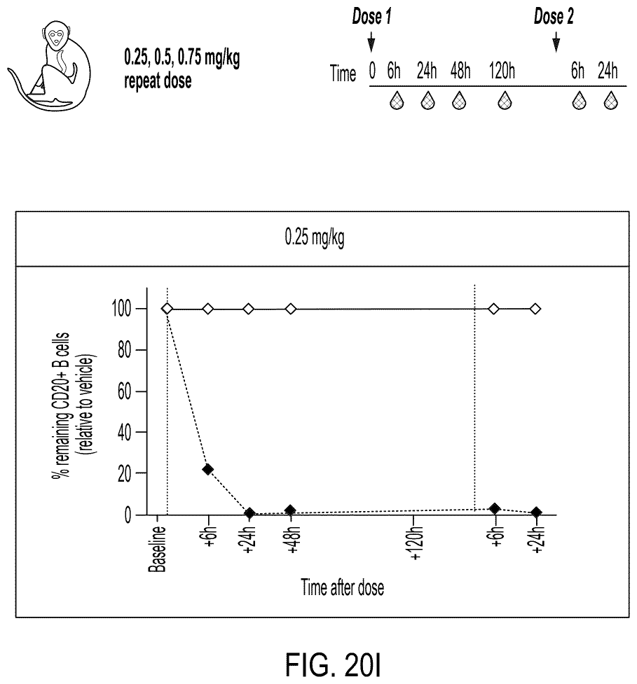

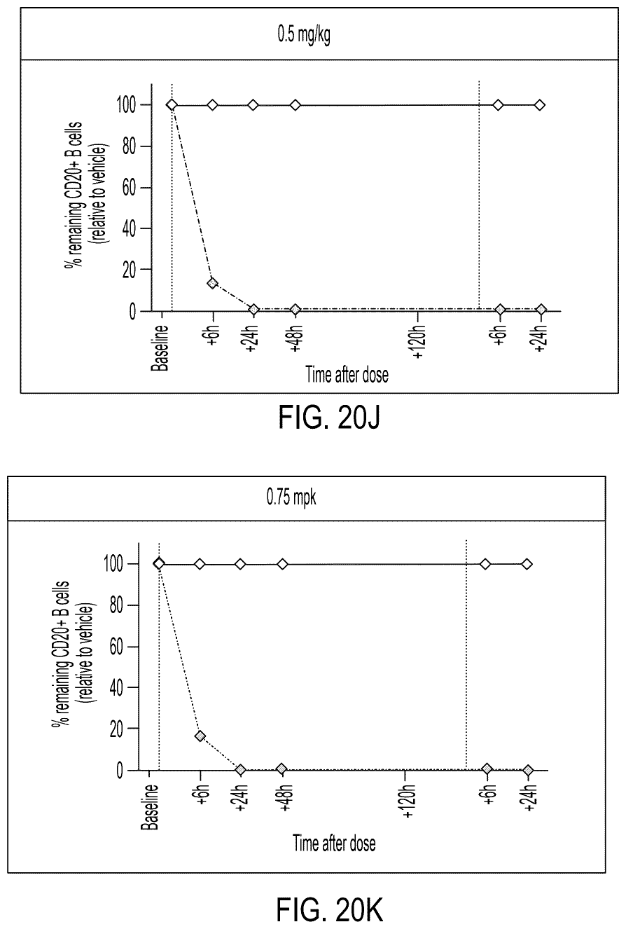

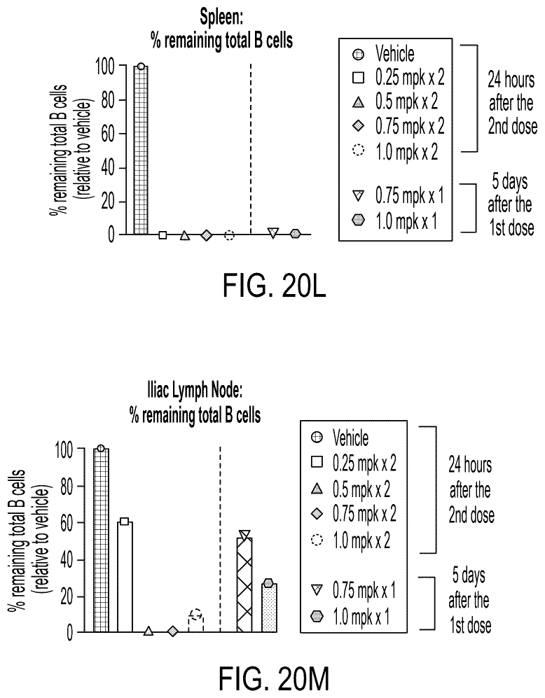

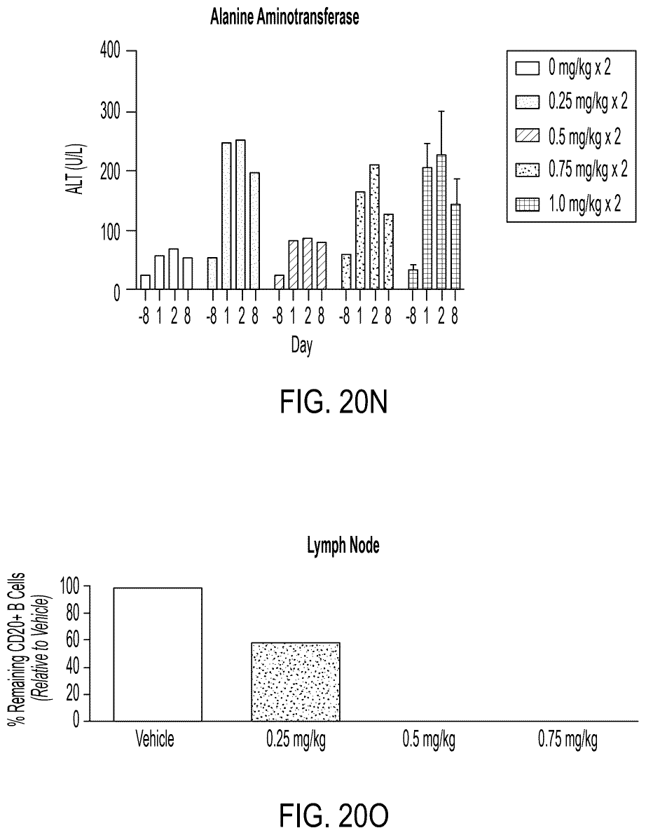

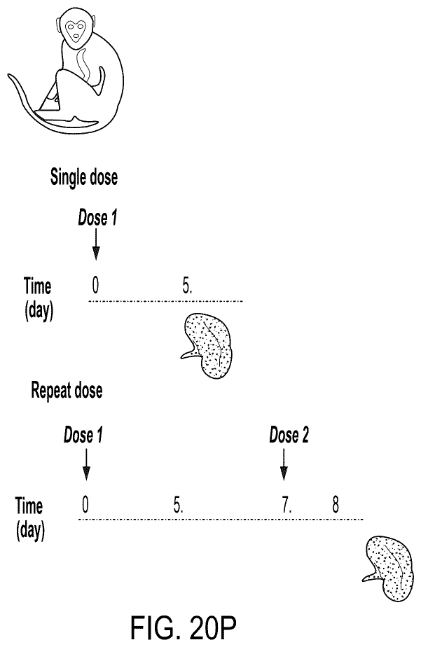

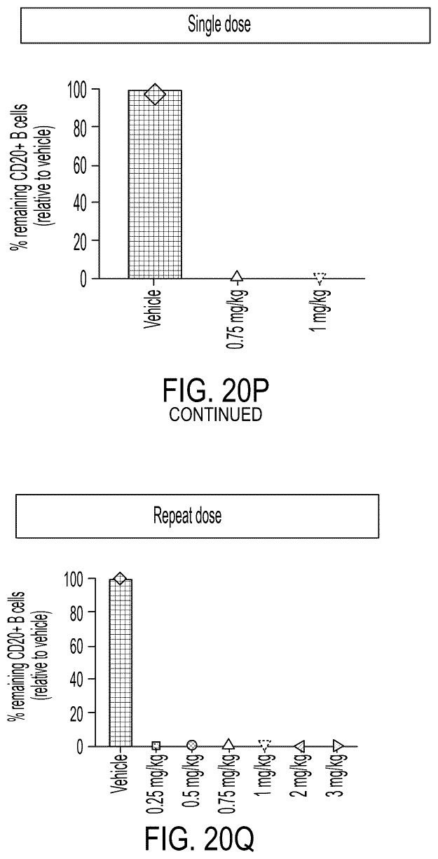

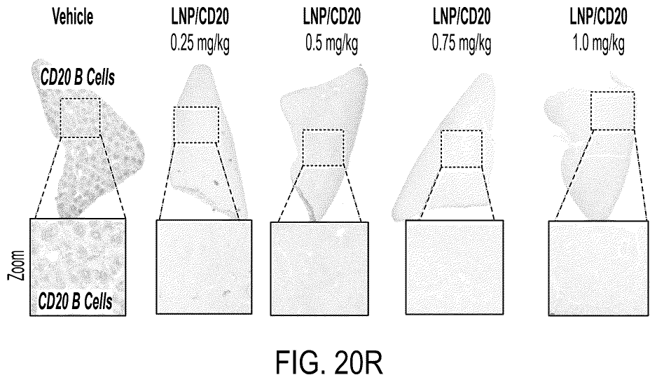

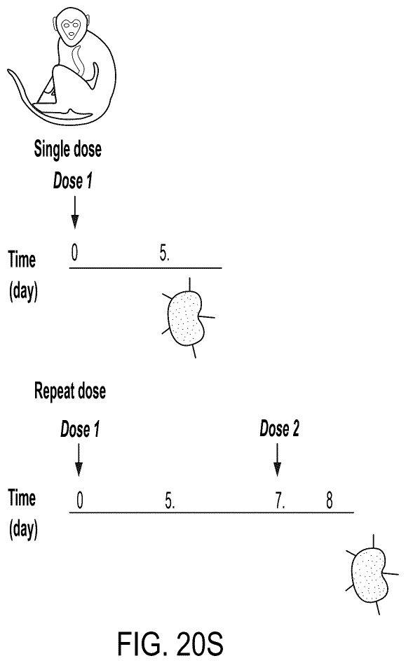

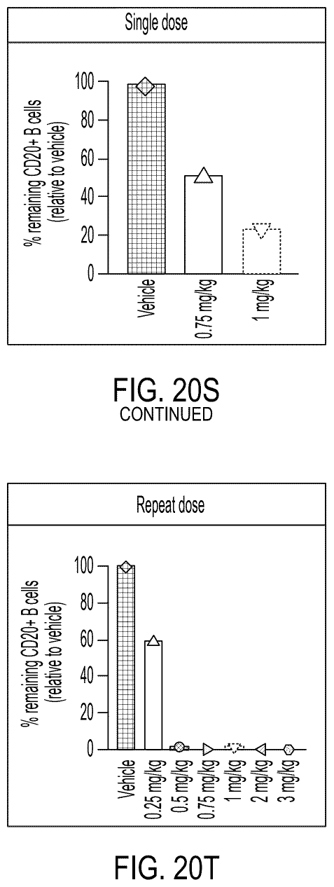

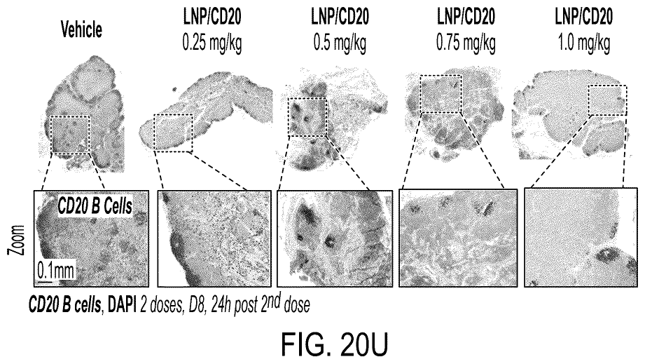

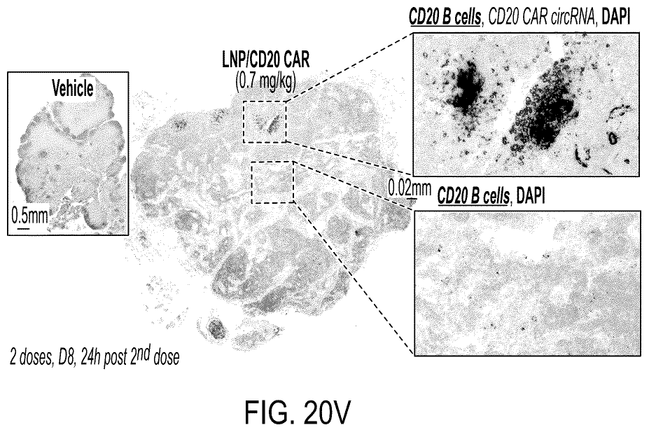

The data in FIG. 20A-FIG. 20C show CAR expression levels from cynomolgus monkeys in Cohort 1 after administration of the circRNAs encoding an anti-CD20 CAR. FIG. 20A is a graph of percent anti-CD20 CAR positive cells as a percentage of CD3+ T cells in blood from the cynomolgus monkeys. The panel shows a time course of CAR expression after each dose of 1 mg/kg, 2 mg/kg, or 3 mg/kg circRNA relative to a vehicle control. This data showed the successful generation of CAR+ T cells after each dose, confirming the re-doseability of the circRNAs encoding an anti-CD20 CAR. In other words, the data showed that the T cells were able to express CAR T cells after each dose of the circRNA.

FIG. 20B is a graph of the number of anti-CD20 CAR positive CD3+ T cells present in each microliter (μL) of blood. The panel shows the number of CAR+ T cells generated and present in blood at each time depicted in the graph. Each cynomolgus monkey was dosed on day 0 and re-dosed on day 7. The number of CAR+ CD3+ T cells was measured by flow cytometry at baseline (e.g., 7 days prior to the dose administration), 6 hours, 24 hours after each dose, as well as 48 hours after the first dose. The data showed significant CAR T cell expansion after the second dose for all doses.

FIG. 20C is a graph of the percentage of anti-CD20 CAR-positive CD3+ T cells present in the deep tissues (iliac lymph node and spleen). Each cynomolgus monkey was dosed on day 0 and day 7 at the 2 mg/kg (Group C) and 3 mg/kg (Group D) doses. The percentage of CAR+ CD3+ T cells was measured by flow cytometry at 24 hours after the second dose. The graphs represent the average of two cynomolgus monkeys per dose.

FIG. 20D and FIG. 20E show levels of B cell depletion in the blood (FIG. 20D) and tissues (FIG. 20E) from cynomolgus monkeys in Cohort 1. FIG. 20D is a graph of the percentage of remaining B cells in the blood over time relative to a vehicle control. Each cynomolgus monkey was dosed on day 0 and day 7 at the 1 mg/kg, 2 mg/kg, or 3 mg/kg doses. The graphs represent the average of two cynomolgus monkeys per dose. The absolute count of B cells present in circulation at each time point was measured by flow cytometry and used to calculate the remaining B cells relative to the number of B cells relative to the vehicle control. The data showed near full depletion as early as 24 hours after the first dose at each dose. In addition, after the second dose, further B cell depletion was achieved at all doses after six hours. For the 1 mg/kg dose, significant B cell depletion was achieved at day 15.

FIG. 20E is a graph of percent remaining B cells in the deep tissue (iliac lymph node and spleen) relative to a vehicle control. Each cynomolgus monkey was dosed on day 0 and day 7 at 1 mg/kg, 2 mg/kg, or 3 mg/kg doses. For the 2 mg/kg and 3 mg/kg groups, the panel shows the percentage of B cells present in the tissues at 24 hours after the second dose. For the 1 mg/kg group, the panel shows the percentage of B cells present in the tissues at 8 days after the second dose. Each data point indicates a different animal. The data showed significant B cell depletion in both tissues for all doses.

The data in FIG. 20F-FIG. 20N show CAR expression levels from cynomolgus monkeys in Cohort 2 after administration of the circRNAs encoding an anti-CD20 CAR. FIG. 20F and FIG. 20G show CAR expression levels from Cohort 2 monkeys. FIG. 20F is a graph of the percentage of anti-CD20 CAR positive cells as a percentage of CD3+ T cells in blood. The panel shows a time course of CAR expression from both the single-dose and double-dose groups. As shown in Table 18, for the single-dose group, the monkeys were administered 0.75 mg/kg or 1 mg/kg doses, and CAR expression was measured at 6 hours, 24 hours, and 48 hours after the dose. For the double-dose groups, the monkeys were administered 0.25 mg/kg, 0.5 mg/kg, 0.75 mg/kg, or 1 mg/kg doses twice (on day 0 and day 7). CAR expression was measured at 6 hours, 24 hours, and 48 hours after the first dose, as well as five days later for the single-dose groups.

FIG. 20G is a graph of the number of anti-CD20 CAR-positive CD3+ T cells present in each microliter (μL) of blood. The panel shows the number of CAR+ T cells generated and present in blood at each time point depicted in the graph. The number of CAR+CD3+ T cells was measured by flow cytometry at baseline before the dose, at 6 hours, and 24 hours after each dose as well as 48 hours after the first dose.

FIG. 20H is a graph of the percentage of remaining B cells in the blood relative to a vehicle control. The panel shows a time course of B cell depletion for both single and double dose groups. For the single dose, the monkeys were administered at either 0.75 mg/kg or 1 mg/kg, and CAR expression was measured at 6 hours, 24 hours, and 48 hours after the dose. For the double-dose groups, the monkeys were administered twice at 0.25 mg/kg, 0.5 mg/kg, 0.75 mg/kg, or 1 mg/kg on day 0 and day 7. The number of B cells present in the blood was measured at 6 hours, 24 hours, and 48 hours after the first dose, as well as five days later (120 hours) for the single-dose groups. In addition, the number of B cells was measured at 6 hours and 24 hours after the second dose. Notably, this data showed that full B cell depletion in the blood was observed in the cynomolgus monkeys 24 hours after administering the first dose of the circRNAs encoding the anti-CD20 CAR, and that this B cell depletion was also achieved after two doses. FIG. 20I-FIG. 20K shows the time course for the 0.25 mg/kg (FIG. 20I), 0.5 mg/kg (FIG. 20J), and 0.75 mg/kg (FIG. 20K) double dosed groups, and (for ease of review) isolates each of the different dose groups from the data shown in FIG. 20H.

FIG. 20L is a graph of the percentage of remaining B cells in the spleen relative to a vehicle control. For the single-dose groups, the monkeys were administered at 0.75 mg/kg or 1 mg/kg doses, and B cell depletion in the spleen was measured at day 5 after the dose. For the double-dose groups, the monkeys were administered twice each at 0.25 mg/kg, 0.5 mg/kg, 0.75 mg/kg, or 1 mg/kg on day 0 and day 7. On day 8, 24 hours after the second dose, the animals were sacrificed to quantify the number of B cells present in the tissue. B cell numbers were used to calculate the percentage of remaining B cell population relative to a vehicle control. The data showed that administering a single dose of circRNA encoding anti-CD20 CAR achieved full B cell depletion, and this B cell depletion was also achieved after a second dose in the lymph node of cynomolgus monkeys.

FIG. 20M is a graph of the percentage of remaining B cells in the iliac lymph node relative to a vehicle control. For the single-dose groups, the monkeys were administered at 0.75 mg/kg or 1 mg/kg, and B cell depletion in the lymph node was measured at day 5 after the dose. For the double-dose groups, the monkeys were administered twice at 0.25 mg/kg, 0.5 mg/kg, 0.75 mg/kg, or 1 mg/kg each on day 0 and day 7. On day 8, 24 hours after the second dose, the monkeys were sacrificed to quantify the number of B cells present in the tissue. B cell numbers were used to calculate the percentage of remaining B cell population relative to a vehicle control. The data showed full B cell depletion in the iliac lymph node of monkeys administered two doses each at 0.75 mg/kg, and robust B cell depletion at 0.5 mg/kg.

FIG. 20N is a graph of exemplary data from Cohorts 1 and 2 for a liver enzyme (alanine aminotransferase, ALT) time course present in blood following repeat administration of circRNA at day 0 and day 7 at doses of 0 mg/kg (vehicle control), 0.25 mg/kg, 0.5 mg/kg, 0.75 mg/kg, and 1.0 mg/kg. This data showed that the doses were safe and well-tolerated in NHPs at doses that produced robust B cell depletion. Only minimal, transient increases in ALT were observed following repeat doses (on Day 0 and Day 8) of doses up to the 1 mg/kg dose.

FIG. 20O is a graph of the percentage of remaining CD20+ B cells in the lymph node relative to a vehicle control in monkeys that were administered 0.25 mg/kg, 0.5 mg/kg, or 0.75 mg/kg circRNA twice on day 0 and day 7, and subsequently B cell depletion in the lymph node was measured 24 hours after the second dose (that is, on day 8). Viable lymphocytes isolated from monkeys were gated using a CD20 marker for quantifying a CD20+ B cell subpopulation by flow cytometry, and CD20+ B cell numbers were used to calculate the percentage of remaining CD20+ B cells relative to a vehicle control.

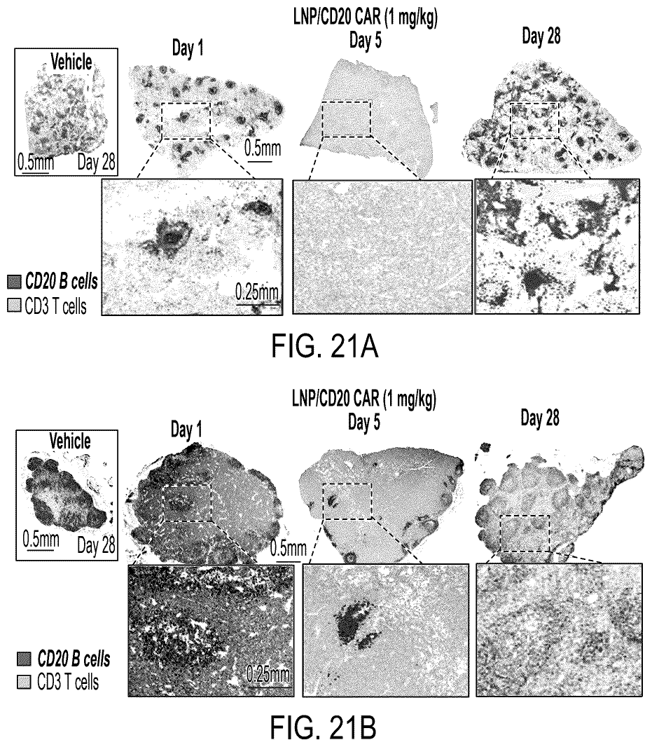

FIG. 20P is a graph showing the percentage of remaining CD20+ B cells in the spleen relative to a vehicle control (top panel). The monkeys in Cohort 1 were administered circRNA encoding anti-CD20 CAR at a single dose of 0.75 mg/kg or 1 mg/kg, and B cell depletion in the spleen was measured at day 5 after the dose (FIG. 21A, bottom panel). CD20+ gated B cell numbers were used to calculate the percentage of remaining CD20+ B cells relative to a vehicle control.

FIG. 20Q is a graph showing the percentage of remaining CD20+ B cells in the spleen relative to a vehicle control. The monkeys (in Cohorts 1 and 2) were administered circRNA encoding anti-CD20 CAR twice each at 0.25 mg/kg, 0.5 mg/kg, 0.75 mg/kg, 1 mg/kg, 2 mg/kg or 3 mg/kg on day 0 and day 7. On day 8, 24 hours after the second dose, the animals were sacrificed to quantify the number of B cells present in the tissue. CD20+ gated B cell numbers were used to calculate the percentage of remaining CD20+ B cell population relative to a vehicle control. The graph combines the NHP data from Cohort 2 (for the 0.25 mg/kg, 0.5 mg/kg, 0.75 mg/kg, and 1 mg/kg doses) and Cohort 1 (for the 2 mg/kg and 3 mg/kg doses).

Further, in this study, in situ hybridization (ISH) was performed on NHP spleen sections collected 24 hours post-dose. Samples were fixed in 10% neutral-buffered formalin, embedded in paraffin, and sectioned at 4 μm-6 μm. Sections were deparaffinized, rehydrated, and permeabilized to allow probe access to target RNA or DNA. After optional acetylation and dehydration, a labeled probe complementary to the target sequence (anti-CD20 CAR) was denatured and applied to each section in the hybridization buffer, then incubated overnight in a humidified chamber at approximately 55° C.-65° C. The next day, slides underwent high-stringency washes and RNase digestion to remove non-specific binding. Hybridized probes were then detected using an antibody followed by chromogenic or fluorescent signal development. Finally, slides were washed, dehydrated, and mounted for imaging. Proper controls against untreated tissue and housekeeping gene probes were included for optimization of permeabilization and hybridization conditions for reliable detection in lymphoid tissue collected post-dose.

FIG. 20R depicts in situ hybridization (ISH) images that were generated 24 hours after the second dose of circRNA was administered to the monkeys. The ISH images showed that after two doses of circRNA, at 0.5 mg/kg or higher, formulated in targeted LNP, full depletion of B cells was seen in the spleen. The CD20 marker stained B cells, and DAPI stained cell nuclei.

FIG. 20S is a graph showing the percentage of remaining CD20+ B cells in the lymph node relative to a vehicle control (bottom panel). The monkeys were administered circRNA encoding anti-CD20 CAR as single dose, and B cell depletion in the lymph node was measured at day 5 after the dose (as illustrated in the top panel). Representative data from the 0.75 mg/kg or 1 mg/kg doses are shown. CD20+ gated B cell numbers were used to calculate the percentage of remaining CD20+ B cells relative to a vehicle control.

FIG. 20T is a graph showing the percentage of remaining CD20+ B cells in the lymph node relative to a vehicle control. The monkeys (Cohorts 1 and 2) were administered circRNA encoding anti-CD20 CAR twice, on day 0 and day 7, each dose at 0.25 mg/kg, 0.5 mg/kg, 0.75 mg/kg, 1 mg/kg, 2 mg/kg or 3 mg/kg. On day 8, 24 hours after the second dose, the animals were sacrificed to quantify the number of B cells present in the tissue. CD20+ gated B cell numbers were used to calculate the percentage of remaining CD20+ B cells relative to a vehicle control. The graph combines the data from Cohort 2 (for the 0.25 mg/kg, 0.5 mg/kg, 0.75 mg/kg, and 1 mg/kg doses) and Cohort 1 (for the 2 mg/kg and 3 mg/kg doses).

Similar to ISH studies described for spleen sections, ISH was also carried out on lymph node sections collected 24 hours post-dose. FIG. 20U depicts ISH images in the lymph node that showed that after two doses of the circRNA encoding anti-CD20 CAR formulated in targeted LNP was administered to the cynomolgus monkeys, robust B cell depletion was observed at day 8, 24 hours after the second dose. The CD20 marker stained B cells, and DAPI stained cell nuclei.

FIG. 20V shows magnified versions of the ISH image for the 0.75 mg/kg dose from FIG. 20U (vehicle control). As shown in FIG. 20V (top right panel), this image showed that although remaining B cells were detected, those B cells (CD20 stained) co-localized with the presence of circRNA (anti-CD20 CAR circRNA stained). As shown in FIG. 20V, although not bound by any mechanism of action, this ISH image suggested that the detection of the circRNA meant that T cells transfected with the circRNA were in the process of depleting B cells, that is, that there was an ongoing B cell depletion phenomenon.

In summary, in this non-human primate study, the in vivo CAR-T approach using circRNA encoding an anti-CD20 CAR for treating, for example, B cell-driven autoimmune diseases showed that treatment with two doses of the circRNA, for example, at 0.75 mg/kg each administered one week apart, resulted in full B cell depletion in the blood, spleen, and lymph node of the cynomolgus monkeys. This full B cell depletion is expected to correlate to a reset of the immune system in autoimmune diseases. The data showed that the circRNA encoding an anti-CD20 CAR was generally well-tolerated.

### Example 21. 28-Day Study of CircRNA (encoding Anti-CD20 CAR) in Targeted LNP Administered by Intravenous Infusion to Cynomolgus Monkeys

In this Example, the objectives of this in vivo NHP study were to evaluate the safety, tolerability, and biological effects of circRNA encoding an anti-CD20 CAR formulated in targeted LNP intravenously administered to cynomolgus monkeys. In one aspect, the CAR comprising an anti-CD20 antigen-binding fragment (an scFv) was a surrogate for a CAR comprising an anti-CD19 antigen-binding fragment. In particular, the study characterized the dose tolerability, pharmacokinetics, and biodistribution of the circRNA encoding an anti-CD20 CAR formulated in targeted LNP by assessing clinical signs, laboratory parameters, immune responses, and tissue findings after administration to cynomolgus monkeys.

As in Example 20, the circRNA administered in this Example comprises a 5′ UTR; an IRES of SEQ ID NO: 1; a coding sequence encoding an anti-CD20 scFv, a CD8a hinge (SEQ ID NO: 285), a CD8a transmembrane domain (SEQ ID NO: 287), a 4-1BB costimulatory domain (SEQ ID NO: 293), and a CD3 zeta signaling domain (SEQ ID NO: 294); and a 3′ UTR comprising one microRNA binding site, specifically, a miR-122 binding site that comprises SEQ ID NO: 267. The circRNA was formulated in targeted LNP, which was conjugated to anti-CD5 targeting moiety VHH319 (SEQ ID NO: 89) via thiol-maleimide conjugation using the cysteine in the conjugation handle (SEQ ID NO: 120). The LNP composition was LNP-18 as described in Table 16, and the ionizable lipid was Compound 7.

The experimental design of this NHP study is shown in Table 19 below. A total of 21 naïve male cynomolgus monkeys were assigned across eight experimental groups, with Group 1 serving as the control. The monkeys received either single (Group 1 and 7-8) or repeat intravenous infusions (Group 2-6) of the circRNA formulated in targeted LNP at dose levels of 0.25 mg/kg, 0.5 mg/kg, 0.75 mg/kg, or 1.0 mg/kg, or vehicle control (1×-PBS).

TABLE 19

| | | | | | Dose | | |
|---|---|---|---|---|---|---|---|
| | Test Article: | Dose Level | | | Concentration | | |
| | CircRNA in | (mg/kg) | | | (mg/mL) | | |
| | targeted | First | Second | Dose | First | Second | |
| Group | LNP | Dose | Dose | Volume | Dose | Dose | No. of Monkeys |
| No. | formulation | (Day 0) | (Day 7) | (mL/kg) | (Day 0) | (Day 7) | (all male) |
| 1 | Vehicle | 0 | — | 5 | 0 | — | 3 |
| | Control | | | | | | |
| 2 | CAR | 0.25 | 0.75 | 5 | 0.05 | 0.15 | 3 |
| 3 | encoding | 0.5 | 0.75 | 5 | 0.1 | 0.15 | 3 |
| 4 | Anti-CD20 in | 0.75 | 0.25 | 5 | 0.15 | 0.05 | 3 |
| 5 | CD5-targeted | 0.75 | 0.5 | 5 | 0.15 | 0.1 | 3 |
| 6 | LNP | 0.75 | 0.75 | 5 | 0.15 | 0.15 | 3 |
| 7 | | 1 | — | 5 | 0.2 | — | 1 |
| 8 | | 1 | — | 5 | 0.2 | — | 2 |
| | | | | | | | Total Monkeys: 21 |

The study endpoints included clinical observations, body weights, hematology, clinical chemistry, and coagulation. In addition, cytokine and complement analysis, anti-drug antibody (ADA) evaluation, and flow cytometry to monitor CAR expression and B-cell depletion were performed. Necropsies were conducted at scheduled timepoints (days 5, 12, and 28). Tissue samples were collected for Ribonucleic Acid (RNA) analysis, in situ hybridization (ISH), histopathology, and flow cytometry of spleen and lymph nodes.

In this study, ISH was performed on NHP lymph node sections collected at day 1, day 5, or day 28 post-dose. Samples were fixed in 10% neutral-buffered formalin, embedded in paraffin, and sectioned at 4 μm-6 μm. Sections were deparaffinized, rehydrated, and permeabilized to allow probe access to target RNA. After optional acetylation and dehydration, a labeled probe complementary to the target sequence (anti-CD20 CAR) was denatured and applied to each section in the hybridization buffer, then incubated overnight in a humidified chamber at approximately 55° C.-65° C. The next day, slides underwent high-stringency washes and RNase digestion to remove non-specific binding. Hybridized probes were then detected using an antibody followed by chromogenic or fluorescent signal development. Finally, slides were washed, dehydrated, and mounted for imaging. Proper controls against untreated tissue and housekeeping gene probes were included for optimization of permeabilization and hybridization conditions for reliable detection in lymphoid tissue collected post-dose.

FIG. 21A depicts ISH images of the spleen from day 1, day 5, and day 28 after a single administration of 1 mg/kg of the circRNA encoding anti-CD20 CAR formulated in targeted LNP to cynomolgus monkeys. A vehicle control at day 28, as well as CD3 marker stained T cells and CD20 marker stained B cells at day 1, day 5 and day 28 are shown. The ISH images showed full B cell depletion at day 5 in the spleen, and re-population of B cells by day 28.

FIG. 21B depicts ISH images of the lymph node from day 1, day 5, and day 28 after a single administration of 1 mg/kg of the circRNA encoding anti-CD20 CAR formulated in targeted LNP to cynomolgus monkeys. An ISH image from the vehicle control generated from day 28, as well as CD3 marker stained T cells and CD20 marker stained B cells at day 1, day 5 and day 28 are shown. The ISH images showed robust B cell depletion at day 5 in the lymph node, and re-population of B cells by day 28.

In summary, the data showed robust or full B cell depletion in the spleen and lymph node after administration of the circRNA encoding anti-CD20 CAR formulated in targeted LNP. In addition, no test article-related adverse clinical signs or changes in body weight related to the circRNA formulated in targeted LNP were observed throughout the study. Overall, the study showed tolerability of the circRNA formulated in targeted LNP in the cynomolgus monkeys.

### Example 22. A 4-Week Intravenous Repeat Dose Study Using a circRNA Encoding an Anti-CD19 CAR Formulated in Targeted LNP in Cynomolgus Monkeys

In this Example, an in vivo study was conducted in non-human primates (NHP) to evaluate repeat dose biodistribution and potential toxicity using circRNA encoding an anti-CD19 CAR formulated in targeted LNP (tLNP), which was administered intravenously to cynomolgus monkeys.

CircRNA encoding an anti-CD19 CAR, as disclosed herein, is not cross-reactive between humans and cynomolgus monkeys. This study was used to evaluate biodistribution and toxicity due to the targeted lipid nanoparticle, without confounding effects from the circRNA payload. The circRNA administered in this Example is described in Tables 7B, 7C and 7D. The circRNA contains a 5′ UTR (SEQ ID NO: 348); an IRES of SEQ ID NO: 1; a coding sequence encoding an anti-CD19 scFv (SEQ ID NO: 264), a CD8a hinge (SEQ ID NO: 285), a CD8a transmembrane domain (SEQ ID NO: 287), a 4-1BB costimulatory domain (SEQ ID NO: 293), and a CD3 zeta signaling domain (SEQ ID NO: 294); and a 3′ UTR (SEQ ID NO: 349) comprising one microRNA binding site, specifically, miR-122 binding site (SEQ ID NO: 40) flanked by an upstream and a downstream spacer (SEQ ID NO: 267, upstream spacer+miR-122 binding site+downstream spacer). The circRNA was formulated in targeted LNP, which was conjugated to anti-CD5 targeting moiety VHH319 (SEQ ID NO: 89) via thiol-maleimide conjugation using the cysteine in the conjugation handle (SEQ ID NO: 120). The LNP composition was LNP-18 as described in Table 16, and the ionizable lipid was Compound 7.

The experimental design of this NHP study is shown in Table 20 below. A total of 15 naïve male cynomolgus monkeys were assigned across three experimental groups, with Group 1 serving as the control. The monkeys received repeat intravenous infusions (Group 2 and 3) of the circRNA formulated in targeted LNP at dose levels of 0.5 mg/kg, or 1.5 mg/kg, or vehicle control.

The monkeys were evaluated over a period of 4 weeks. The animals were dosed weekly on Days 1 and 8 via a 60-minute intravenous infusion. Control animals received treatment with a vehicle that did not contain any circRNA or targeted LNP. Animals were pre-treated with immunosuppressants. Doses were administered according to Table 20 below. Animals were euthanized on Day 9 or 29.

TABLE 20
Study Design of 4-week repeat dosing Study with anti-CD19 CAR

| | Test Article: | | | | | | | |
|---|---|---|---|---|---|---|---|---|
| | CircRNA in | | Dose | Dose | Interim Study | | Main Study | |
| Group | targeted LNP | Dose level | volume^{a} | concentration | No. of | No. of | No. of | No. of |
| No. | formulation | (mg/kg/dose) | (mL/kg) | (mg/mL) | males | females | males | females |
| 1 | Vehicle/Control | 0 | 5 | 0 | — | 1 | 1 | 2 |
| 2 | CAR encoding | 0.5 | 5 | 0.1 | 1 | 1 | 2 | 2 |
| 3 | Anti-CD19 in | 1.5 | 5 | 0.3 | 1 | 1 | 2 | 2 |
| | CD5-targeted | | | | | | | |
| | LNP | | | | | | | |

^{a}Based on most recent body weight measurement

Alanine amino transferase (ALT) levels were measured over 4 weeks in vehicle/control treated monkeys and monkeys administered 0.5 mg/kg/dose in two doses or 1.5 mg/kg/dose in two doses. Results are shown for vehicle/control (Table 21A), 0.5 mg/kg/dose (Table 21B) or 1.5 mg/kg/dose (Table 21C). The included data summarizes all ALT study values in NHPs surviving to scheduled necropsy.

TABLE 21A
ALT Levels of Monkeys Treated with Vehicle Control (0 mg/kg of
CircRNA encoding anti-CD19 CAR in Targeted LNP

| | Dose (mg/kg) | | | |
|---|---|---|---|---|
| | 0 mg/kg | 0 mg/kg | 0 mg/kg | 0 mg/kg |
| | Animal Number | | | |
| | 1 | 2 | 3 | 4 |
| | Day(s) Relative to Start of Dosing | | | |
| | ALT (U/L) | ALT (U/L) | ALT (U/L) | ALT (U/L) |
| −7 | 44 | 40 | 72 | 49 |
| 2 | 240 | 101 | 184 | 106 |
| 3 | 215 | 93 | 165 | 115 |
| 8 | 109 | 74 | 74 | 74 |
| 9 | 187 | 79 | 123 | 107 |
| 10 | 169 | — | 119 | 112 |
| 15 | 52 | — | 45 | 62 |
| 22 | 28 | — | 28 | 48 |
| 29 | 32 | — | 27 | 44 |

TABLE 21B
ALT Levels of Monkeys Treated with 0.5 mg/kg/dose
of CircRNA encoding anti-CD19 CAR in Targeted LNP

| | Dose (mg/kg) | | | | | |
|---|---|---|---|---|---|---|
| | 0.5 mg/ | 0.5 mg/ | 0.5 mg/ | 0.5 mg/ | 0.5 mg/ | 0.5 mg/ |
| | kg/dose | kg/dose | kg/dose | kg/dose | kg/dose | kg/dose |
| Day(s) | Animal Number | | | | | |
| Relative | 5 | 6 | 7 | 8 | 9 | 10 |
| to Start of | ALT | ALT | ALT | ALT | ALT | ALT |
| Dosing | (U/L) | (U/L) | (U/L) | (U/L) | (U/L) | (U/L) |
| −7 | 44 | 30 | 47 | 61 | 41 | 37 |
| 2 | 116 | 80 | 174 | 169 | 123 | 116 |
| 3 | 122 | 90 | 156 | 201 | 131 | 223 |
| 8 | 81 | 49 | 134 | 125 | 68 | 107 |
| 9 | 114 | 82 | 151 | 176 | 153 | 130 |
| 10 | — | 62 | 121 | — | 106 | 103 |
| 15 | — | 30 | 46 | — | 42 | 64 |
| 22 | — | 17 | 23 | — | 29 | 45 |
| 29 | — | 16 | 32 | — | 26 | 42 |

TABLE 21C
ALT Levels of Monkeys Treated with 1.5 mg/kg/dose of CircRNA
encoding anti-CD19 CAR in Targeted LNP

| | Dose (mg/kg) | | | | |
|---|---|---|---|---|---|
| | 1.5 mg/ | 1.5 mg/ | 1.5 mg/ | 1.5 mg/ | 1.5 mg/ |
| | kg/dose | kg/dose | kg/dose | kg/dose | kg/dose |
| | Animal Number | | | | |
| | 11 | 12 | 13 | 14 | 15 |
| | Day(s) Relative to Start of Dosing | | | | |
| | ALT (U/L) | ALT (U/L) | ALT (U/L) | ALT (U/L) | ALT (U/L) |
| −7 | 36 | 44 | 37 | 47 | 40 |
| 2 | 105 | 137 | 119 | 234 | 166 |
| 3 | 113 | 140 | 105 | 243 | 221 |
| 8 | 63 | 67 | 76 | 104 | 106 |
| 9 | 90 | 110 | 98 | 183 | 148 |
| 10 | — | 150 | 98 | — | 134 |
| 15 | — | 45 | 36 | — | 48 |
| 22 | — | 34 | 40 | — | 30 |
| 29 | — | 28 | 26 | — | 28 |

The results in Table 21 showed that alanine amino transferase (ALT) levels were comparable to control and liver biodistribution was effectively minimized. Furthermore, no microscopic signs of test-article related liver pathology were observed by a board-certified veterinary pathologist.

In summary, the results showed that the circRNA encoding an anti-CD19 CAR formulated in targeted LNP did not result in elevated ALT levels in the cynomolgus monkeys. Elevated ALT levels would have been indicative of liver toxicity. It is contemplated that single microRNA binding site designed in the circRNA and the anti-CD5 targeting moiety that was conjugated to the LNP effectively minimized off-target liver biodistribution and associated liver toxicity.

### EQUIVALENTS AND SCOPE

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

1-54. (canceled)

55. A circular ribonucleic acid (circRNA) comprising in order:
(a) a 5′ exon fragment and a 5′ untranslated region (UTR),
(b) an Internal Ribosomal Entry Site (IRES) comprising a sequence at least 85% identical to any one of SEQ ID NO: 1-11,
(c) a coding sequence encoding a chimeric antigen receptor (CAR) comprising an anti-CD19 antibody or antigen-binding fragment thereof,
(d) a 3′ UTR and a 3′ exon fragment, and
(e) a single microRNA binding site within the 5′ UTR or the 3′ UTR.

56-117. (canceled)

118. A precursor RNA comprising, from 5′ to 3′ end:
(i) a 5′ intron fragment and a 5′ exon fragment,
(ii) a 5′ untranslated region (UTR),
(iii) an IRES,
(iv) a coding sequence encoding a chimeric antigen receptor (CAR) comprising an anti-CD19 antibody or antigen-binding fragment thereof,
(v) a 3′ untranslated region (UTR), and
(vi) a 3′ exon fragment and 3′ intron fragment,
wherein the 5′ and the 3′ intron fragments are derived from a Twort-ORF142 Group I intron,
wherein the 3′ UTR comprises a microRNA binding site flanked by an upstream spacer and a downstream spacer, and
wherein each of the upstream spacer and the downstream spacer comprise no more than five (5) consecutive adenines (As).

119-146. (canceled)

147. A circular RNA therapeutic for targeted delivery comprising:
(i) a circular RNA (circRNA) comprising an IRES and a coding sequence encoding a chimeric antigen receptor (CAR), the CAR comprising an anti-CD19 antibody or antigen-binding fragment thereof, wherein the circRNA is encapsulated in a lipid nanoparticle (LNP), and
(ii) a targeting moiety conjugated to the LNP, wherein the targeting moiety comprises an anti-CD5 VHH comprising CDR1, CDR2, and CDR3 sequences, wherein CDR1 sequence comprises SEQ ID NO: 64, the CDR2 sequence comprises SEQ ID NO: 65, and the CDR3 sequence comprises SEQ ID NO: 66.

148. The circRNA therapeutic of claim 147, wherein the anti-CD5 VHH comprises a sequence having at least 95% identity to SEQ ID NO: 89.

149. The circRNA therapeutic of claim 148, wherein the anti-CD5 VHH has a length of 110 to 130 amino acids.

150. The circRNA therapeutic of claim 147, wherein the anti-CD5 VHH sequence is SEQ ID NO: 89.

151. The circRNA therapeutic of claim 147, wherein the LNP comprises one or more ionizable lipids at 30 to 49 mol %, one or more phospholipids at 5 to 40 mol %, one or more sterol lipids at 15 to 50 mol %, a PEG lipid at 0 to 10 mol %, and a conjugating lipid at 0.01-1 mol %, wherein the targeting moiety is attached to the conjugating lipid and the conjugating lipid is a conjugating PEG-lipid.

152. The circRNA therapeutic of claim 151, wherein the LNP comprises one or more ionizable lipids at 30 to 45 mol %, one or more phospholipids at 15 to 35 mol %, one or more sterol lipids at 30 to 50 mol %, a PEG lipid at 0 to 5 mol %, and a conjugating lipid at 0.01-1 mol %, wherein the targeting moiety is attached to the conjugating lipid and the conjugating lipid is a conjugating PEG-lipid.

153. The circRNA therapeutic of claim 151, wherein the one or more ionizable lipids is

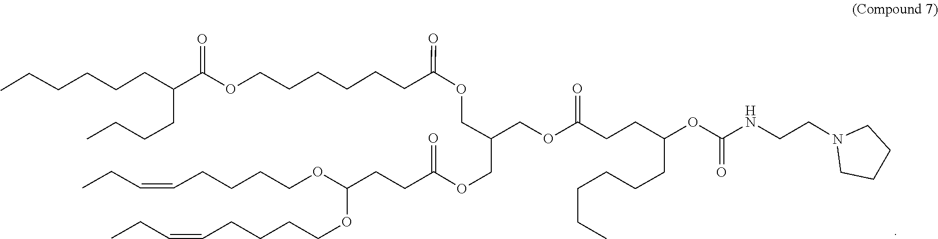 [SMILES: CC/C=C\CCCCOC(CCC(=O)OCC(COC(=O)CCCCCCOC(=O)C(CCCC)CCCCCC)COC(=O)CCC(CCCCCC)OC(=O)NCCN1CCCC1)OCCCC/C=C\CC]

154. The circRNA therapeutic of claim 152, wherein the one or more ionizable lipids is Compound 7.

155. The circRNA therapeutic of claim 153, wherein the PEG lipid is DMG-PEG 2000.

156. The circRNA therapeutic of claim 154, wherein the PEG lipid is DMG-PEG 2000.

157. The circRNA therapeutic of claim 155, wherein the conjugating PEG lipid is DSPE-PEG 2000 maleimide.

158. The circRNA therapeutic of claim 156, wherein the conjugating PEG lipid is DSPE-PEG 2000 maleimide.

159. The circRNA therapeutic of claim 157, wherein the one or more phospholipids is DSPC.

160. The circRNA therapeutic of claim 158, wherein the one or more phospholipids is DSPC.

161. The circRNA therapeutic of claim 159, wherein the one or more sterol lipids is cholesterol.

162. The circRNA therapeutic of claim 160, wherein the one or more sterol lipids is cholesterol.

163. The circRNA therapeutic of claim 161, wherein the anti-CD5 VHH is covalently attached to a conjugation handle, wherein the conjugation handle is conjugated to the conjugating PEG-lipid in the LNP via a thiol-maleimide conjugation between the conjugating PEG-lipid and the conjugation handle.

164. The circRNA therapeutic of claim 162, wherein the anti-CD5 VHH is covalently attached to a conjugation handle, wherein the conjugation handle is conjugated to the conjugating PEG-lipid in the LNP via a thiol-maleimide conjugation between the conjugating PEG-lipid and the conjugation handle.

165. The circRNA therapeutic of claim 163, wherein the conjugation handle is 5-10 amino acids in length.

166. The circRNA therapeutic of claim 163, wherein the conjugation handle comprises SEQ ID NO: 120.

167. The circRNA therapeutic of claim 164, wherein the conjugation handle comprises SEQ ID NO: 120.

168. The circRNA therapeutic of claim 163, wherein the sequence of the VHH together with its attached conjugation handle is SEQ ID NO: 122.

169. The circRNA therapeutic of claim 164, wherein the sequence of the VHH together with its attached conjugation handle is SEQ ID NO: 122.

170. The circRNA therapeutic of claim 164, wherein the IRES comprises a sequence of SEQ ID NO: 1.

171. The circRNA therapeutic of claim 170, wherein the circRNA comprises a microRNA binding site.

172. The circRNA therapeutic of claim 171, wherein the circRNA comprises a microRNA-122 (miR-122) binding site.

173. The circRNA therapeutic of claim 147, the circRNA comprising, in order:
(a) a 5′ exon fragment and a 5′ untranslated region (UTR),
(b) the Internal Ribosome Entry Site (IRES) comprising a sequence of SEQ ID NO: 1,
(c) a coding sequence encoding a chimeric antigen receptor (CAR) comprising an anti-CD19 antibody or antigen-binding fragment thereof,
(d) a 3′ UTR and a 3′ exon fragment, and
(e) a single miR-122 binding site within the 5′ UTR or the 3′ UTR.

174. The circRNA therapeutic of claim 173, wherein the 3′ UTR comprises the single miR-122 binding site.

175. The circRNA therapeutic of claim 147, wherein the circRNA sequence is at least 95% identical to SEQ ID NO: 264.

176. The circRNA therapeutic of claim 169, wherein the circRNA sequence is at least 95% identical to SEQ ID NO: 264.

177. The circRNA therapeutic of claim 176, wherein the circRNA sequence is SEQ ID NO: 264.

178. A method of treating a human patient with autoimmune disease, the method comprising administering the circRNA therapeutic of claim 147 to the patient.

179. A method of depleting B cells in a patient in need thereof, the method comprising administering the circRNA therapeutic of claim 147 to the patient.

180. A method of treating a human patient with autoimmune disease, the method comprising administering the circRNA therapeutic of claim 161 to the patient.

181. A method of depleting B cells comprising administering the circRNA therapeutic of claim 161 to the patient.

182. A method of treating a human patient with autoimmune disease, the method comprising administering the circRNA therapeutic of claim 169 to the patient.

183. A method of depleting B cells in a patient in need thereof, the method comprising administering the circRNA therapeutic of claim 169 to the patient.

184. A method of treating a human patient with autoimmune disease, the method comprising administering the circRNA therapeutic of claim 176 to the patient.

185. A method of depleting B cells in a patient in need thereof, the method comprising administering the circRNA therapeutic of claim 176 to the patient.

186. A method of treating a human patient with autoimmune disease, the method comprising administering the circRNA therapeutic of claim 177 to the patient.

187. A method of depleting B cells in a patient in need thereof, the method comprising administering the circRNA therapeutic of claim 177 to the patient.

188. A circRNA therapeutic comprising
(i) a circRNA comprising a coding sequence encoding an anti-CD19 CAR, wherein the circRNA is encapsulated in an LNP,
(ii) the LNP, wherein the LNP comprises Compound 7 at 39 mol %, DSPC at 20 mol %, cholesterol at 39 mol %, DMG-PEG 2000 lipid at 1.9 mol %, and DSPE-PEG 2000-maleimide lipid at 0.1 mol %, and
(iii) a VHH with a conjugation handle comprising the sequence of SEQ ID NO: 122, wherein the VHH with the conjugation handle is attached to the LNP via thiol-maleimide conjugation of the cysteine in the conjugation handle with the DSPE-PEG 2000-maleimide lipid.

189. The circRNA therapeutic of claim 188, wherein the circRNA sequence is SEQ ID NO: 264.

190. A method of treating a human patient with autoimmune disease, the method comprising administering the circRNA therapeutic of claim 189 to the patient.

191. A method of depleting B cells in a patient in need thereof, the method comprising administering the circRNA therapeutic of claim 189 to the patient.
